(19) United States
(12) Patent Application Publication
(10) Pub. No.: US 2021/0371403 A1
(43) Pub. Date: Dec. 2, 2021

(54) SMALL MOLECULES TARGETING MUTANT MAMMALIAN PROTEINS

(71) Applicant: Jnana Therapeutics, Inc., Boston, MA (US)

(72) Inventors: Giovanni Muncipinto, Cambridge, MA (US); Lyn H. Jones, Winchester, MA (US); Terry J. Rettenmaier, Braintree, MA (US); John A. Malona, Lexington, MA (US)

(21) Appl. No.: 17/278,043

(22) PCT Filed: Sep. 20, 2019
(86) PCT No.: PCT/US2019/052187

§ 371 Date: Mar. 19, 2021

Related U.S. Application Data
(60) Provisional application No. 62/734,613, filed on Sep. 21, 2018.

(51) Int. Cl. C07D 403/12; C07C 279/10; C07D 213/56; C07D 239/26; C07D 213/64; C07D 233/24; C07D 263/32; C07D 233/88; C07D 239/16; C07D 235/02; C07D 231/12; C07D 229/02; C07D 401/12

(52) U.S. Cl. CPC C07D 403/12; C07C 279/10; C07D 213/56; C07D 239/26; C07D 213/64; C07D 233/24; C07D 401/12; C07D 233/88; C07D 239/16; C07D 235/02; C07D 231/12; C07D 229/02; C07D 263/32

(57) ABSTRACT

Disclosed are compounds, compositions, and methods useful for treating or preventing a disease or disorder associated with a mutation in a protein.

### RELATED APPLICATIONS

This application claims the benefit of priority to U.S. Provisional Patent Application No. 62/734,613, filed Sep. 21, 2018.

### BACKGROUND

Creatine transporter deficiency (CTD) has been reported to be the most common cerebral creatine deficiency syndrome (CCDS). Creatine transporter deficiency is an X-linked disorder caused by mutations in the SLC6A8 gene. The SLC6A8 gene, located on the short arm of the sex chromosome, provides instructions for making a protein that transports the compound creatine into cells. Creatine is needed for the body to store and use energy properly. People with CTD have intellectual disability, which can range from mild to severe, and delayed speech development. Some affected individuals develop behavioral disorders such as attention deficit hyperactivity disorder or autistic behaviors that affect communication and social interaction. They may also experience seizures, Children with CTD may experience slow growth and exhibit delayed development of motor skills such as sitting and walking. CTD is difficult to treat because the actual transporter responsible for transporting creatine to the brain and muscles is defective, There is no current standard of care.

### SUMMARY

One aspect of the invention provides compounds, compositions, and methods useful for treating or preventing a disease or disorder associated with a SLC6A8 mutation.

Accordingly, provided herein is a compound having the structure of Formula (I) or (II)

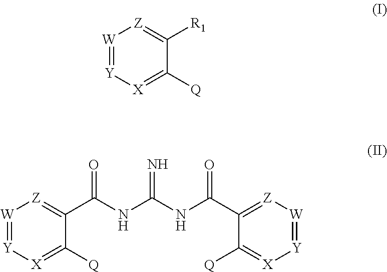

wherein

X, Y, W, and Z are independently selected from N and C(R); provided that no more than two of X, Y, W, and Z are N;

if Z and W, or W and Y, or Y and X are C(R), then any two adjacent instances of R taken together may form a fused 3-8 membered ring;

Q is OH, —NHSO₂R′, —COOH, —C(O)NHSO₂R″, —SO₂NHC(O)R″, tetrazolyl, or —CRₓR_{y}OH;

R is independently selected from H, alkyl, alkenyl, alkynyl, cycloalkyl, heteroalkyl, cycloheteroalkyl, substituted or unsubstituted aryl, substituted or unsubstituted -alkylene-aryl, substituted or unsubstituted heteroaryl, substituted or unsubstituted -alkylene-heteroaryl, haloalkyl, halocycloalkyl, halocycloheteroalkyl, —O-alkyl, —O-haloalkyl, —O-cycloalkyl, —N-alkyl, —N-haloalkyl, —N-cycloalkyl, —S-alkyl, —S-haloalkyl, —S-cycloalkyl, —O-heteroalkyl, —O-cycloheteroalkyl, —N-heteroalkyl, —N-cycloheteroalkyl, —S-heteroalkyl, —S-cycloheteroalkyl, —O-aryl, —N-aryl, —S-aryl, —O-heteroaryl, —N-heteroaryl, —S-heteroaryl, substituted or unsubstituted —O-alkylene-aryl, substituted or unsubstituted —N-alkylene-aryl, substituted or unsubstituted —S-alkylene-aryl, substituted or unsubstituted —O-alkylene-heteroaryl, substituted or unsubstituted —N-alkylene-heteroaryl, substituted or unsubstituted —S-alkylene-heteroaryl, halide, —CN, —NO₂, —S(O)Rₐ, —S(O)₂Rₐ, —C(O)Rₐ, —C(O)₂Rₐ, —C(O)NRₐR_{b}, OH, and C(O)NR′C(NR′)NRₐR_{b};

R′ is H, alkyl, or aryl;

R″ is alkyl or aryl;

Rₐ and R_{b} are independently H, alkyl, alkenyl, alkynyl, substituted or unsubstituted aryl, cycloalkyl, heteroalkyl, haloalkyl, cycloheteroalkyl, halocycloalkyl, halocycloheteroalkyl, substituted or unsubstituted heteroaryl, substituted or unsubstituted -alkylene-aryl, substituted or unsubstituted -alkylene-heteroaryl or Rₐ and R_{b} taken together with the nitrogen atom to which they are attached may form a 3-8 membered ring;

Rₓ and R_{y} are independently H, F, alkyl, aryl, or haloalkyl;

R₁ is

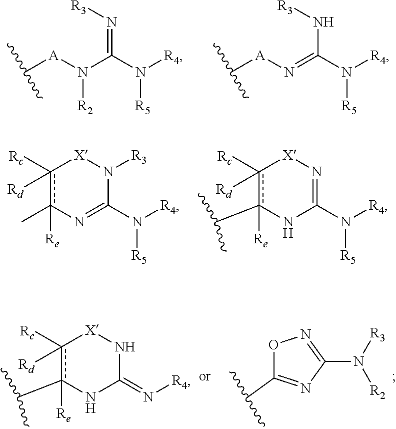

R₂, R₃, R₄, and R₅ are independently selected from H, alkyl, alkenyl, alkynyl, heteroalkyl, cycloalkyl, haloalkyl, halocycloalkyl, cycloheteroalkyl, substituted or unsubstituted aryl, substituted or unsubstituted -alkylene-aryl, substituted or unsubstituted heteroaryl, substituted or unsubstituted -alkylene-heteroaryl, substituted or unsubstituted 5-12 membered ring, alkylenealkoxy, haloalkyl, —CN, —C(O)Rₐ, and —C(O)NRₐR_{b}; provided that (i) no more than one of R₂, R₃, R₄, and R₅ is —CN, (ii) no more than one of R₂, R₃, R₄, and R₅ is —C(O)Rₐ, and (iii) no more than one of R₂, R₃, R₄, and R₅ is —C(O)NRₐR_{b};

R₃ and R₄ taken together may form a 5-8 membered ring;

R₂ and R₅ taken together may form a 5-8 membered ring;

R₄ and R₅ taken together may form a 5-8 membered ring;

R_{c} is H, or alkyl;

R_{d} and Rₑ are independently absent, H, or alkyl;

[symbol] represents a single bond or a double bond; if [symbol] is a double bond, then R_{d} and Rₑ are absent;

A is absent, —CH₂—, —C(O)—, —C(S)—, —S(O)₂—, or —CR_{f}R_{g}—;

X′ is absent, —CH₂—, —C(O)—, —C(S)—, or —S(O)₂—; and

R_{f} and R_{g} are independently selected from H, alkyl, alkenyl, alkynyl substituted or unsubstituted aryl, substituted or unsubstituted heteroaryl, substituted or unsubstituted -alkylene-aryl, substituted or unsubstituted -alkylene-heteroaryl, and halide; or R_{f} and R_{g} taken together may form a spirocyclic 3-8 membered ring, or heterospirocyclic 3-8 membered ring.

One aspect of the invention relates to compounds of Formula (III):

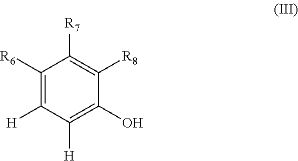

wherein:

R₆ is selected from H, alkyl, cycloalkyl, heteroalkyl, cycloheteroalkyl, substituted or unsubstituted aryl, substituted or unsubstituted -alkylene-aryl, substituted or unsubstituted heteroaryl, substituted or unsubstituted -alkylene-heteroaryl, haloalkyl, halocycloalkyl, halocycloheteroalkyl, —O-alkyl, —O-haloalkyl, —O-cycloalkyl, —N-alkyl, —N-haloalkyl, —S-alkyl, —O-heteroalkyl, —N-heteroalkyl, —S-heteroalkyl, —O-aryl, —N-aryl, —S-aryl, —S-haloalkyl, —S-cycloalkyl, —O-heteroaryl, —O-cycloheteroalkyl, —N-heteroaryl, —N-cycloalkyl, —N-cycloheteroalkyl, —S-cycloheteroalkyl, —S-heteroaryl, halide, —CN, —NO₂, —S(O)Rₐ, —S(O)₂Rₐ, —C(O)Rₐ, —C(O)₂Rₐ, and —C(O)NRₐR_{b};

Rₐ and R_{b} are independently H, alkyl, alkenyl, alkynyl substituted or unsubstituted aryl, cycloalkyl, heteroalkyl, haloalkyl, cycloheteroalkyl, halocycloalkyl, halocycloheteroalkyl, substituted or unsubstituted heteroaryl, substituted or unsubstituted -alkylene-aryl, substituted or unsubstituted -alkylene-heteroaryl or Rₐ and R_{b} taken together with the nitrogen atom to which they are attached may form a 3-8 membered ring;

R₇ is H, halide, alkyl, or aryl;

R₈ is

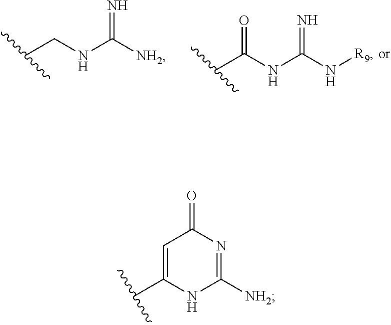

and

R₉ is selected from H, alkyl, alkenyl, alkynyl, heteroalkyl, cycloalkyl, haloalkyl, halocycloalkyl, cycloheteroalkyl, substituted or unsubstituted aryl, substituted or unsubstituted -alkylene-aryl, substituted or unsubstituted heteroaryl, substituted or unsubstituted -alkylene-heteroaryl, —CN, —C(O)Rₐ, and —C(O)NRₐR_{b}.

Another aspect of the invention relates to methods of treating or preventing a disease or disorder associated with a SLC6A8 mutation, comprising administering to a subject in need thereof an effective amount of a compound of the invention.

In some embodiments, the invention relates to methods of increasing cellular trafficking of a creatine transporter, comprising administering to a subject in need thereof an effective amount of a compound of the invention.

In some embodiments, the invention relates to methods of correcting a defect in cellular creatine transporter function, comprising administering to a subject in need thereof an effective amount of a compound of the invention.

In certain embodiments, the subject is a mammal. In certain embodiments, the mammal is a human.

The invention provides several additional advantages. The prophylactic and therapeutic methods described herein are also effective for treating creatine transporter deficiency and associated symptoms. In some embodiments, the therapeutic method is effective in treating motor dysfunction, intellectual disability, language delay, speech delay, seizures, behaviors associated with autism and attention deficit hyperactivity disorder, fatigue, muscular hypotonia, low weight gain, and gastrointestinal and cardiac disorders.

In some embodiments, the therapeutic method is effective in treating inflammatory diseases. In some embodiments, the inflammatory disease is acute. In some embodiments, the inflammatory disease is chronic. In some embodiments, the inflammatory disease is selected from inflammatory bowel diseases (for example, ulcerative colitis or Crohn's disease), multiple sclerosis, psoriasis, arthritis, rheumatoid arthritis, osteoarthritis, juvenile arthritis, psoriatic arthritis, reactive arthritis, ankylosing spondylitis, cryopyrin associated periodic syndromes, Muckle-Wells syndrome, familial cold auto-inflammatory syndrome, neonatal-onset multisystem inflammatory disease, TNF receptor associated periodic syndrome, acute and chronic pancreatitis, atherosclerosis, gout, ankylosing spondylitis, fibrotic disorders (for example, hepatic fibrosis or idiopathic pulmonary fibrosis), nephropathy, sarcoidosis, scleroderma, anaphylaxis, diabetes (for example, diabetes mellitus type 1 or diabetes mellitus type 2), diabetic retinopathy, Still's disease, vasculitis, sarcoidosis, pulmonary inflammation, acute respiratory distress syndrome, wet and dry age-related macular degeneration, autoimmune hemolytic syndromes, autoimmune and inflammatory hepatitis, autoimmune neuropathy, autoimmune ovarian failure, autoimmune orchitis, autoimmune thrombocytopenia, silicone implant associated autoimmune disease, Sjogren's syndrome, familial Mediterranean fever, systemic lupus erythematosus, vasculitis syndromes (for example, temporal, Takayasu's and giant cell arteritis, Behçet's disease or Wegener's granulomatosis), vitiligo, secondary hematologic manifestation of autoimmune diseases (for example, anemias), drug-induced autoimmunity, Hashimoto's thyroiditis, hypophysitis, idiopathic thrombocytic pupura, metal-induced autoimmunity, myasthenia gravis, pemphigus, autoimmune deafness (for example, Meniere's disease), Goodpasture's syndrome, Graves' disease, HW-related autoimmune syndromes, Gullain-Barre disease, Addison's disease, anti-phospholipid syndrome, asthma, atopic dermatitis, Celiac disease, Cushing's syndrome, dermatomyositis, idiopathic adrenal adrenal atrophy, idiopathic thrombocytopenia, Kawasaki syndrome, Lambert-Eaton Syndrome, pernicious anemia, pollinosis, polyarteritis nodosa, primary biliary cirrhosis, primary sclerosing cholangitis, Raynaud's, Reiter's Syndrome, relapsing polychondritis, Schmidt's syndrome, thyrotoxidosis, sepsis, septic shock, endotoxic shock, exotoxin-induced toxic shock, gram negative sepsis, toxic shock syndrome, glomerulonephritis, peritonitis, interstitial cystitis, hyperoxia-induced inflammations, chronic obstructive pulmonary disease (COPD), vasculitis, graft vs. host reaction (for example, graft vs. host disease), allograft rejections (for example, acute allograft rejection or chronic allograft rejection), early transplantation rejection (for example, acute allograft rejection), reperfusion injury, pain (for example, acute pain, chronic pain, neuropathic pain, or fibromyalgia), chronic infections, meningitis, encephalitis, myocarditis, gingivitis, post surgical trauma, tissue injury, traumatic brain injury, enterocolitis, sinusitis, uveitis, ocular inflammation, optic neuritis, gastric ulcers, esophagitis, peritonitis, periodontitis, dermatomyositis, gastritis, myositis, polymyalgia, pneumonia and bronchitis.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features, objects, and advantages of the invention will be apparent from the detailed description, and from the claims.

### BRIEF DESCRIPTION OF THE DRAWINGS

FIG. 1 is a table summarizing trafficking and correction data for exemplary compounds of the invention.

FIG. 2 is a table summarizing trafficking and correction data for exemplary compounds of the invention.

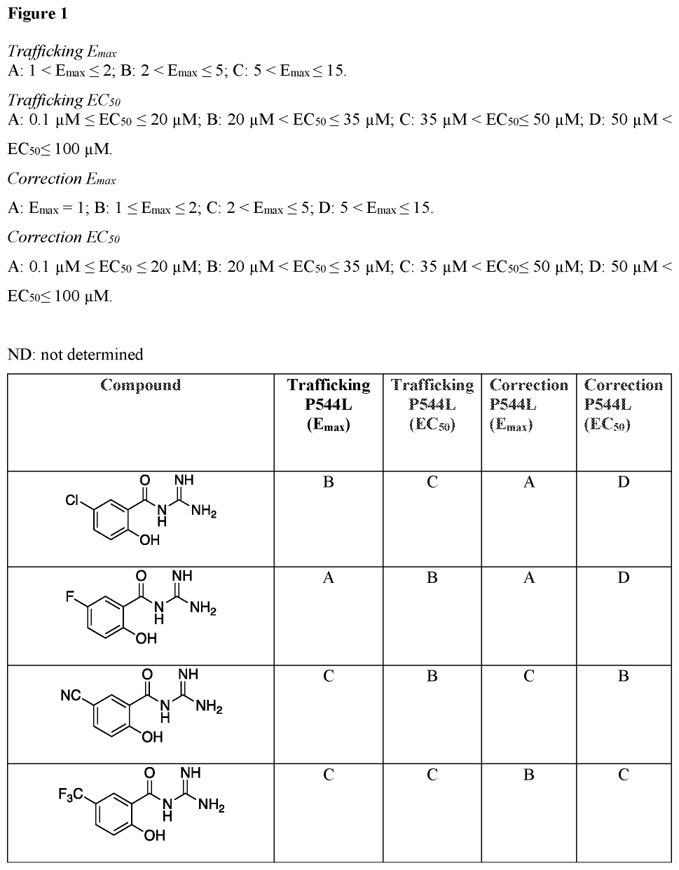

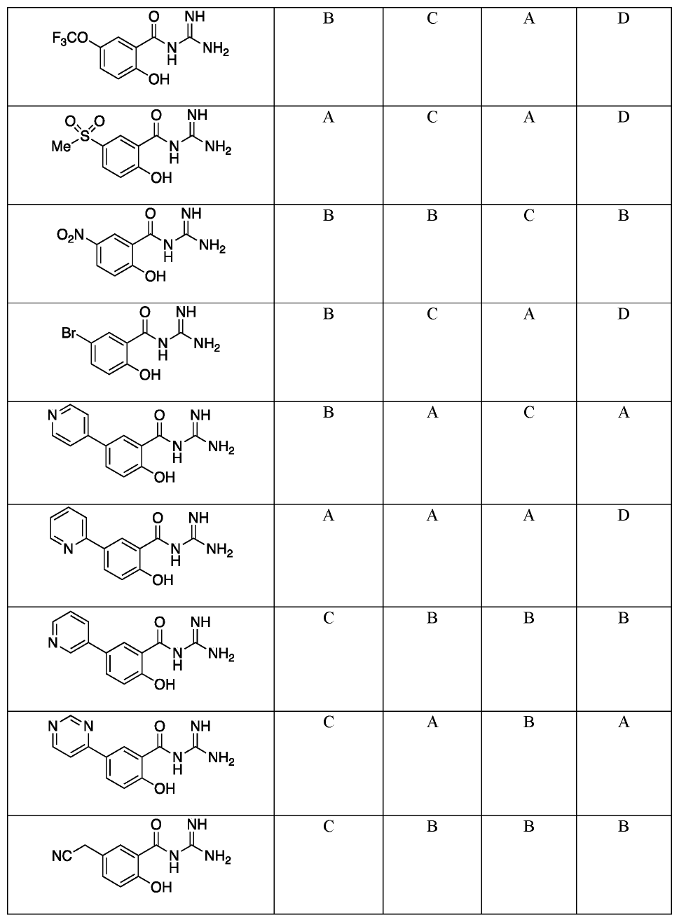

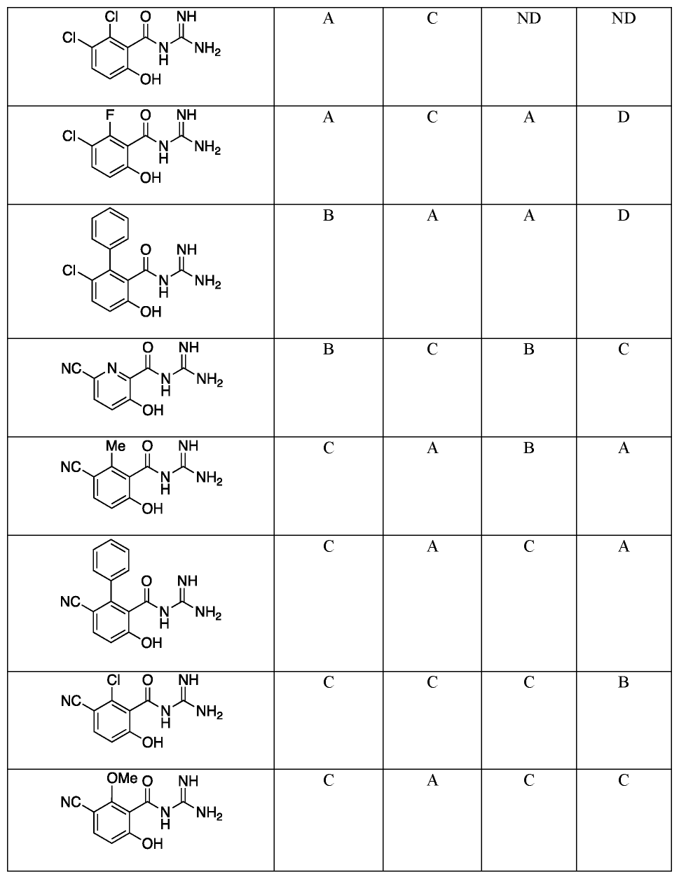

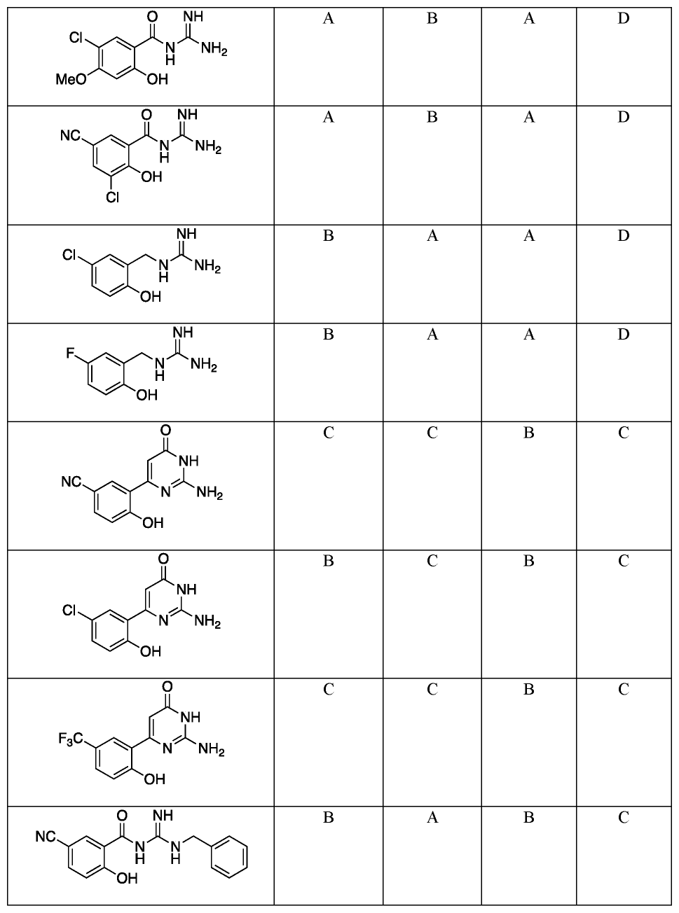

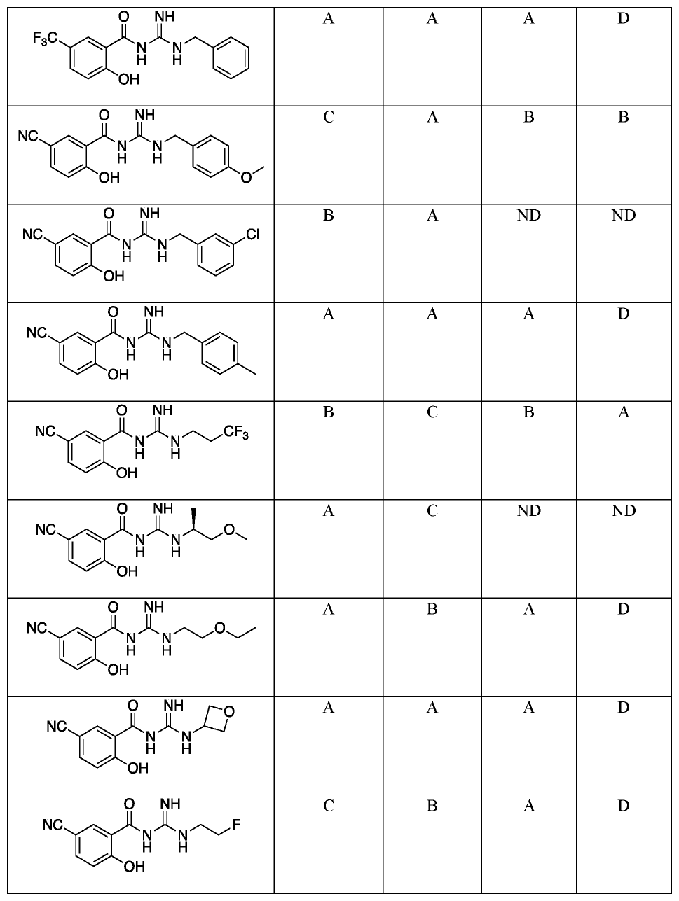

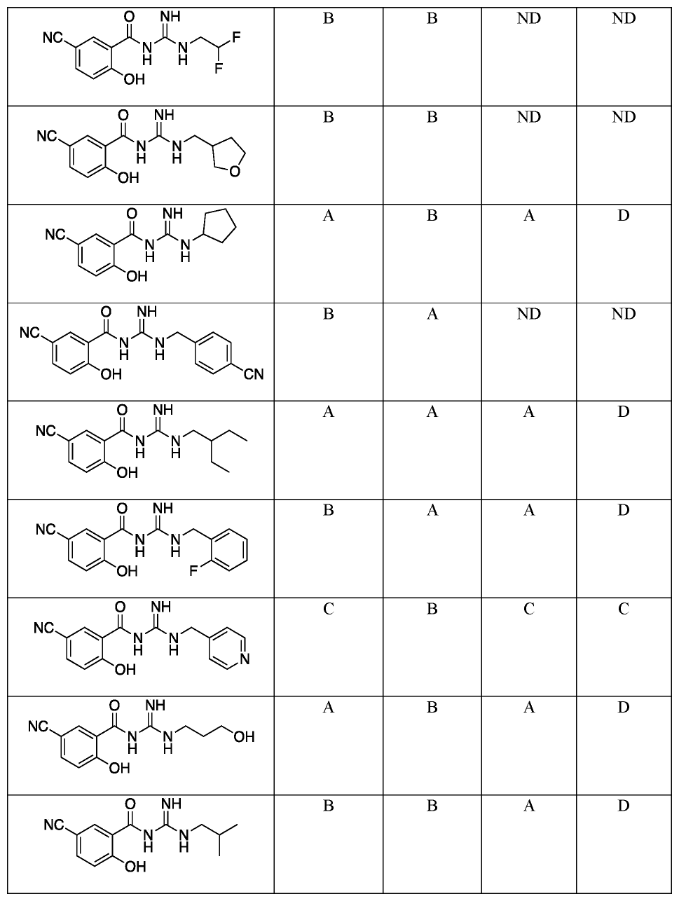

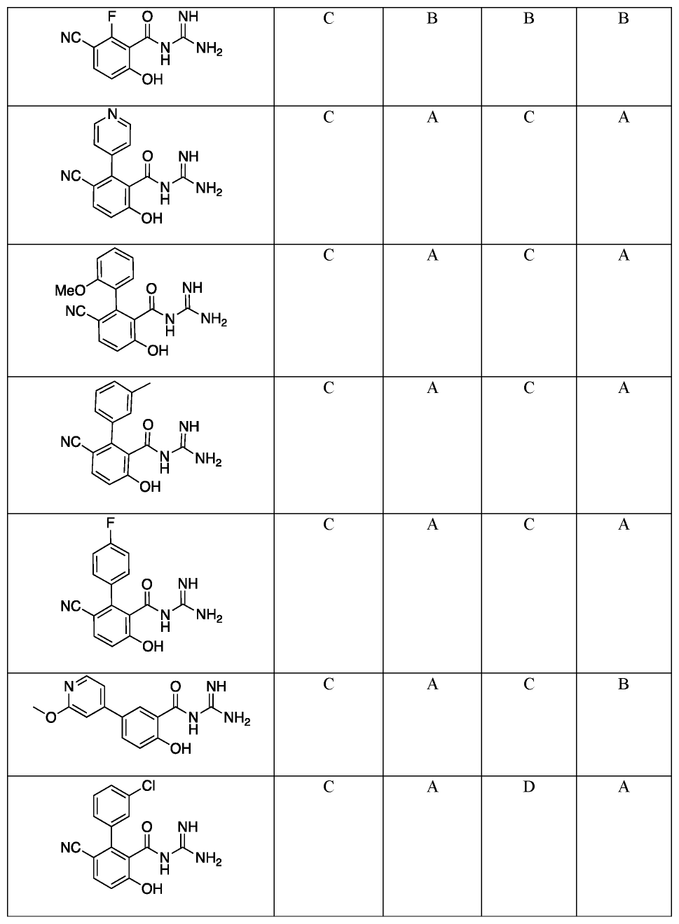

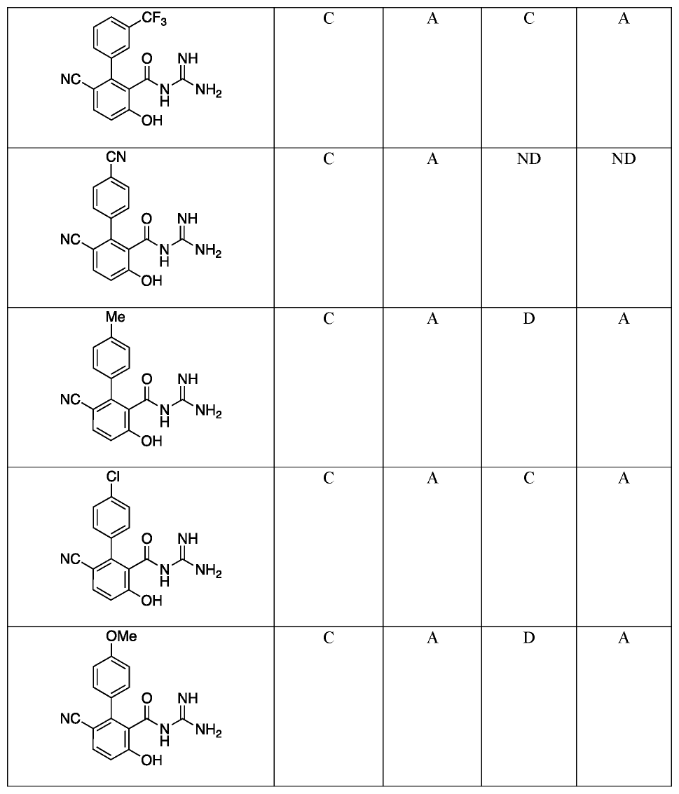

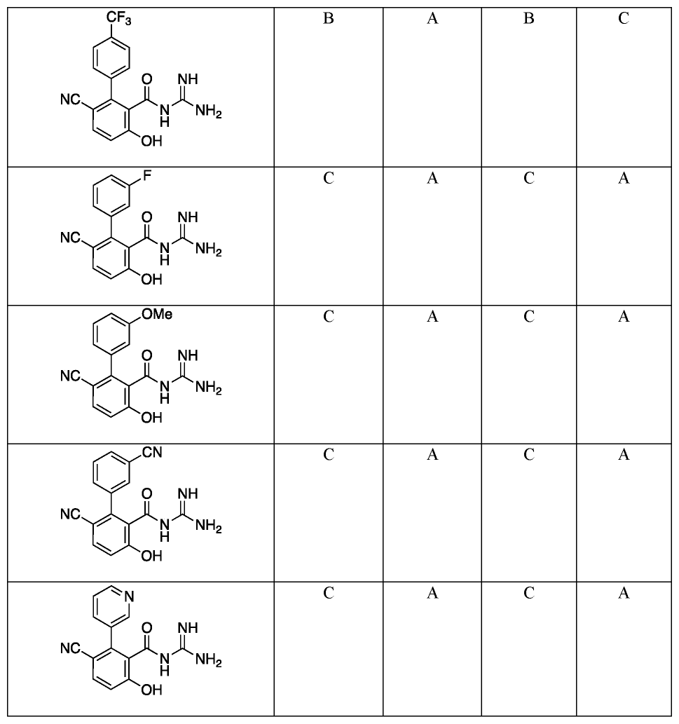

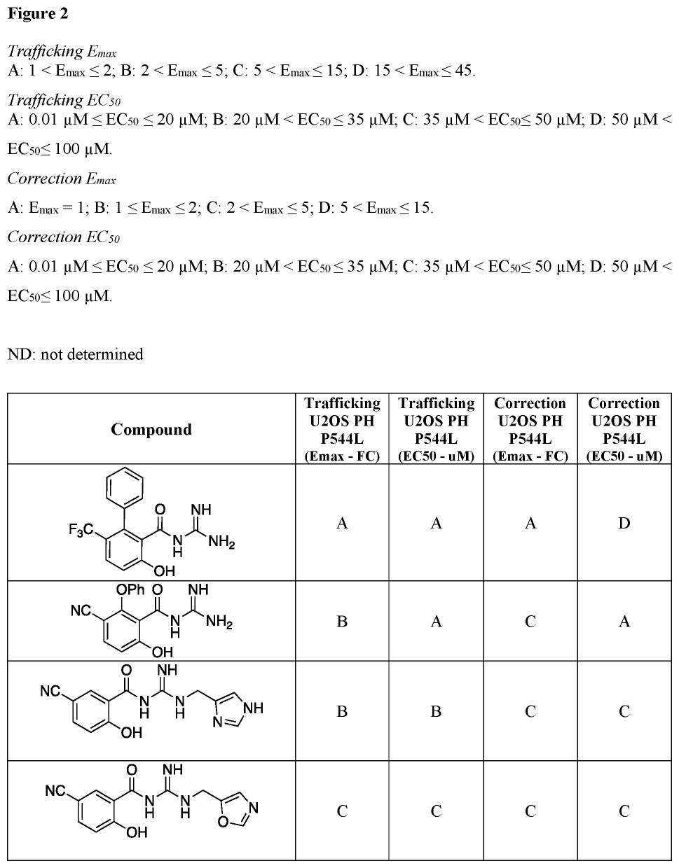

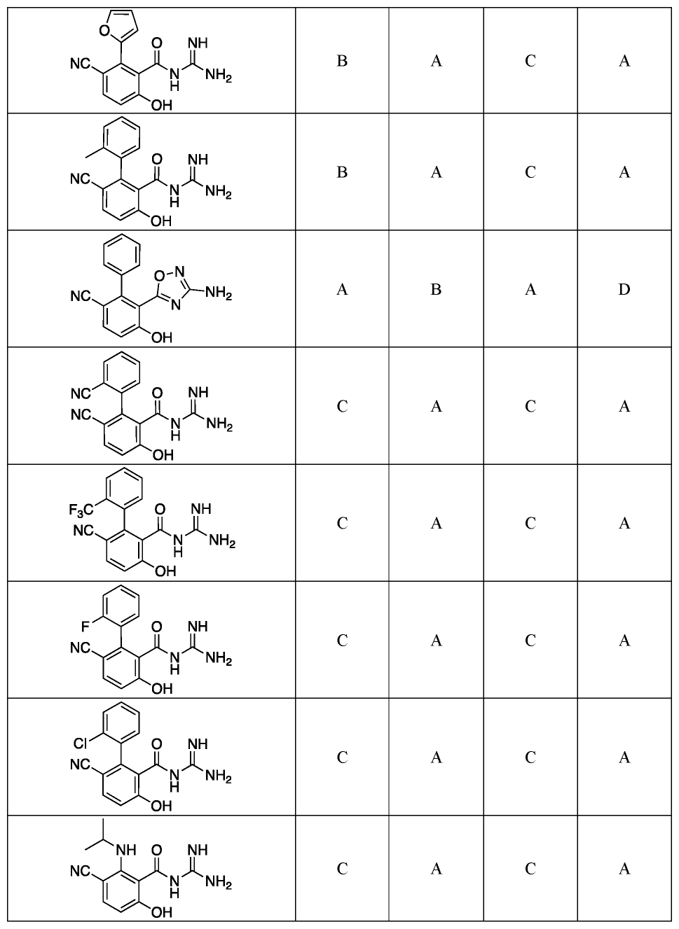

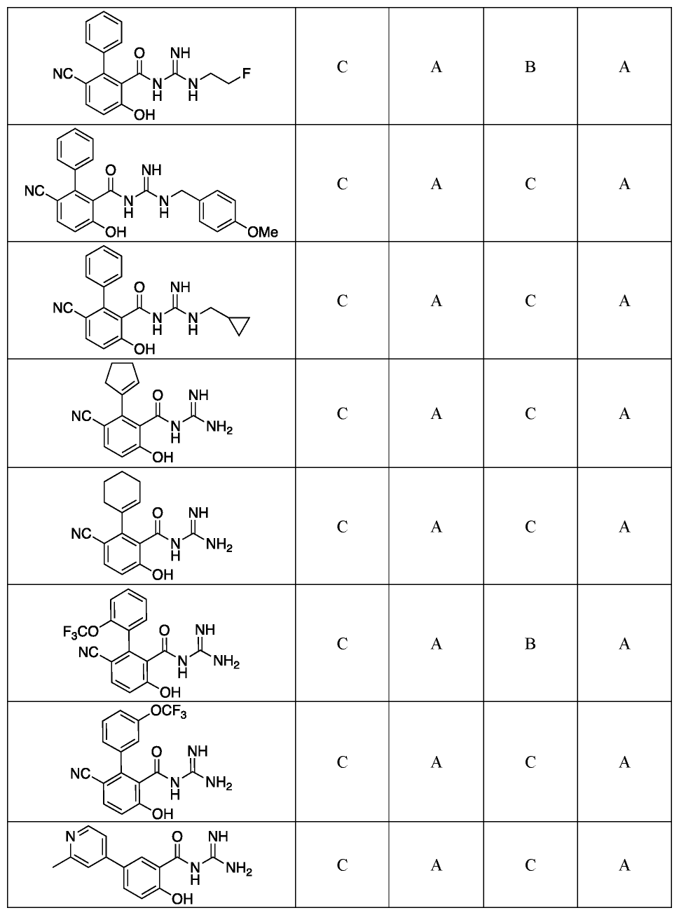

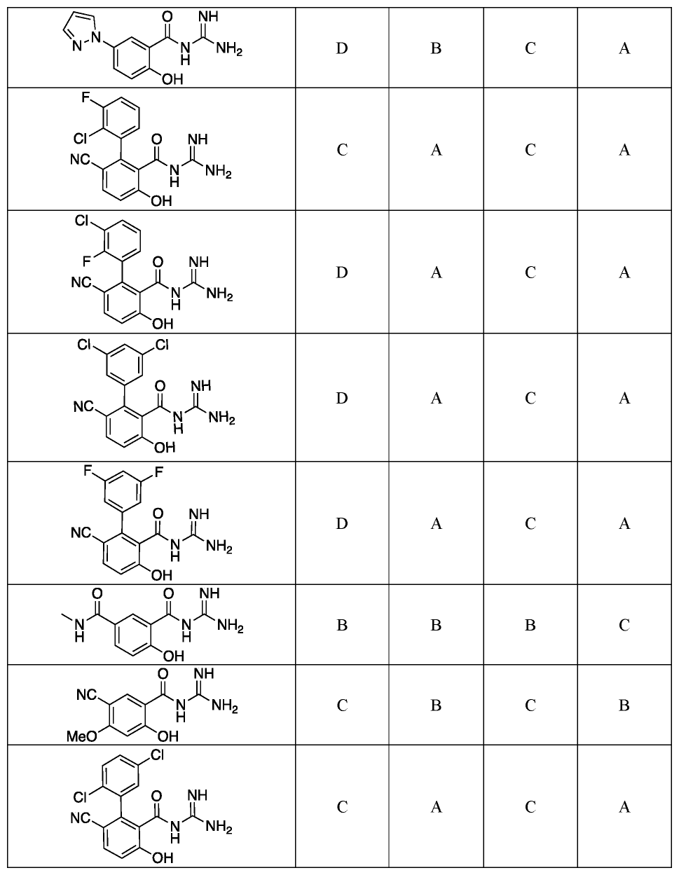

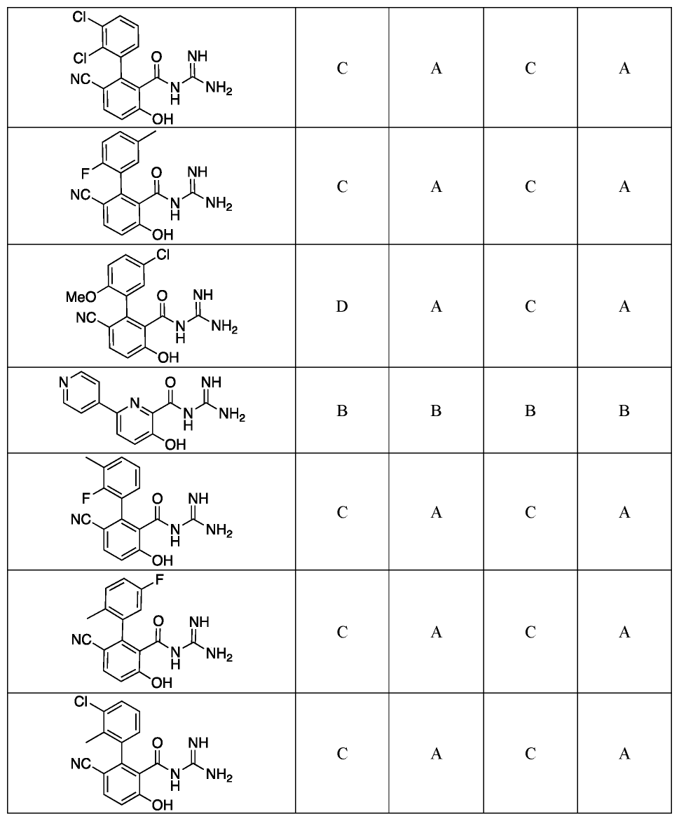

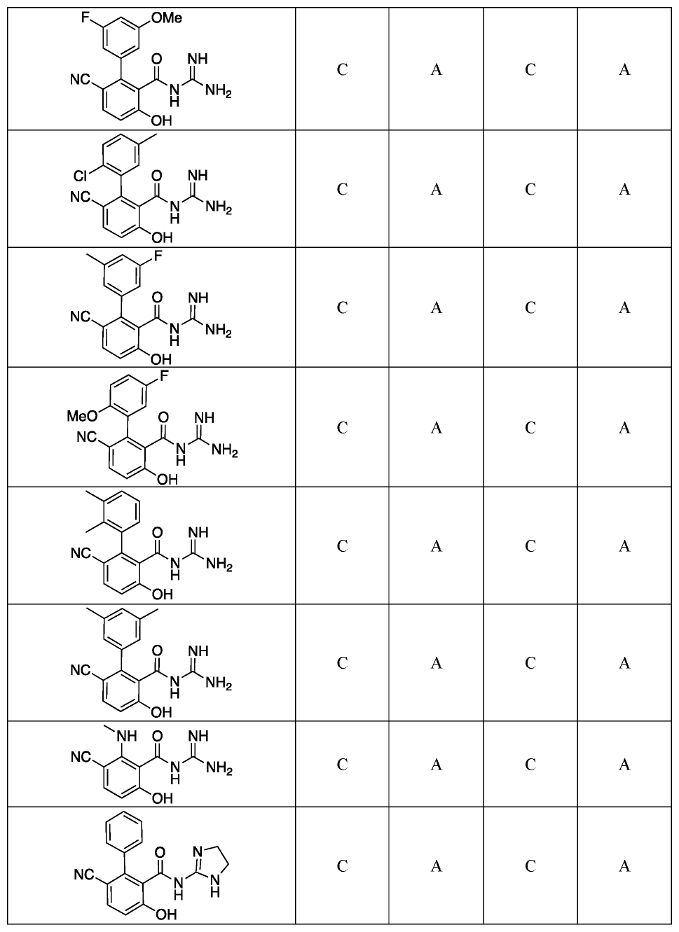

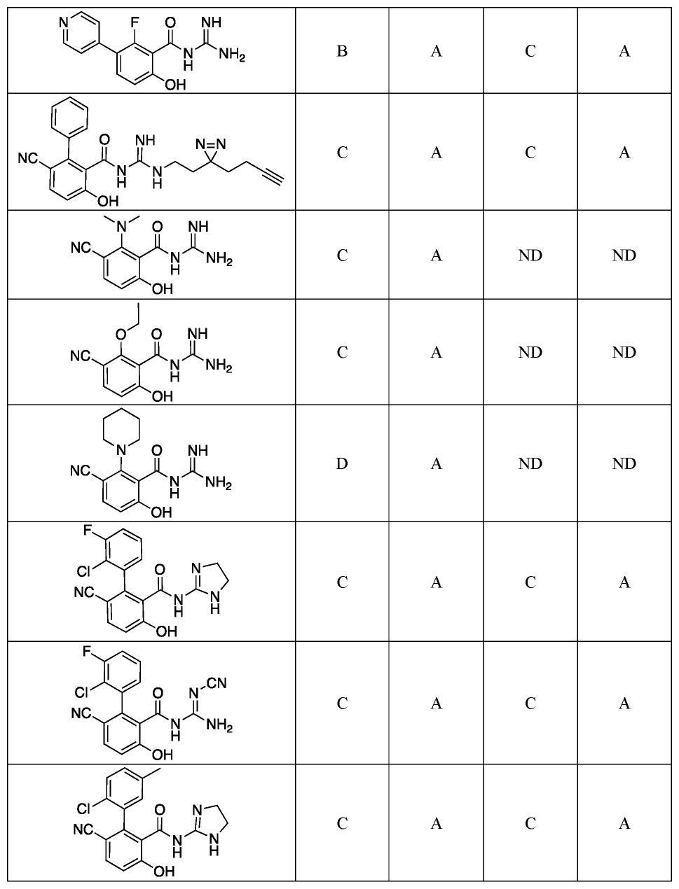

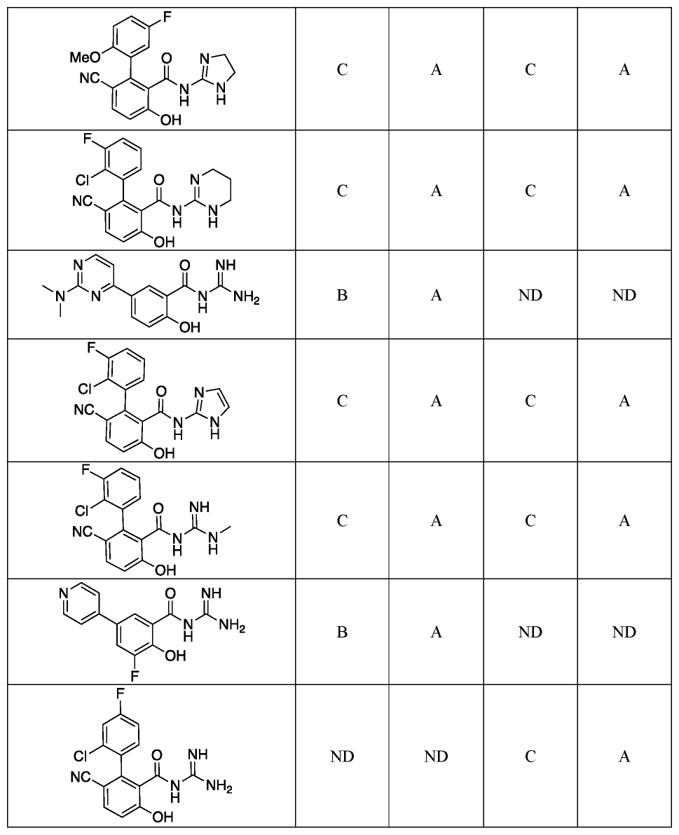

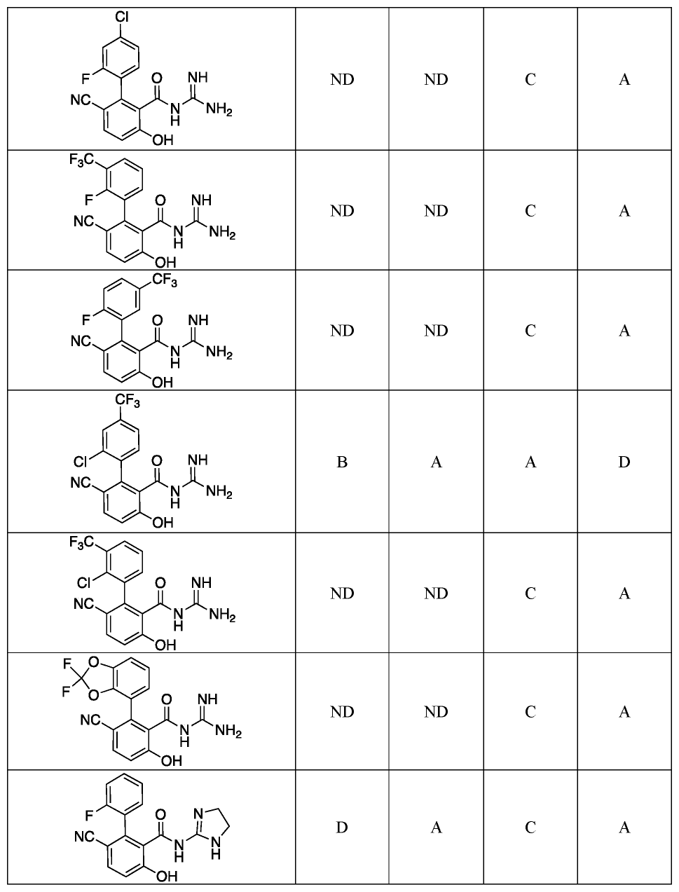

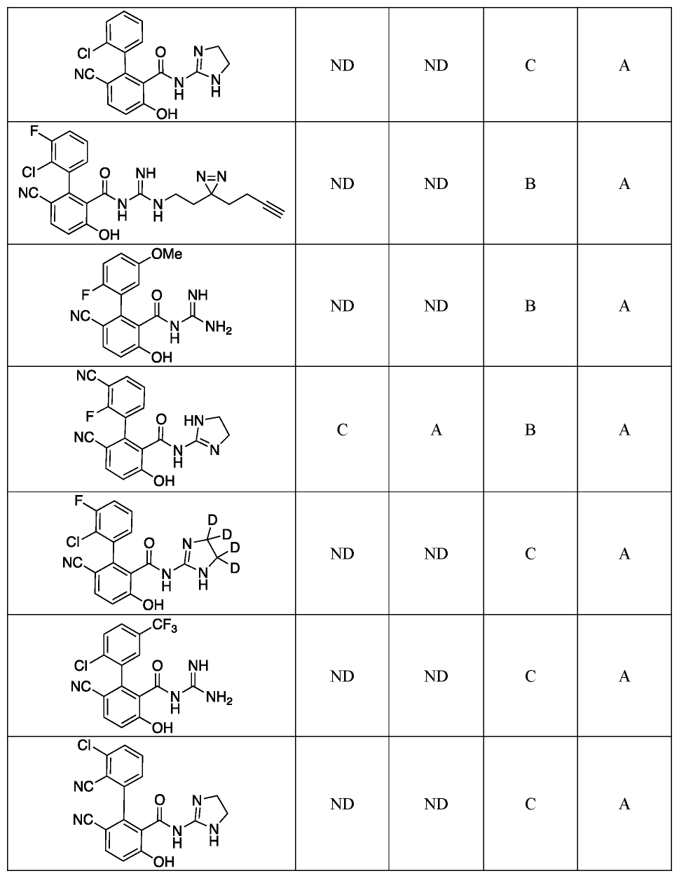

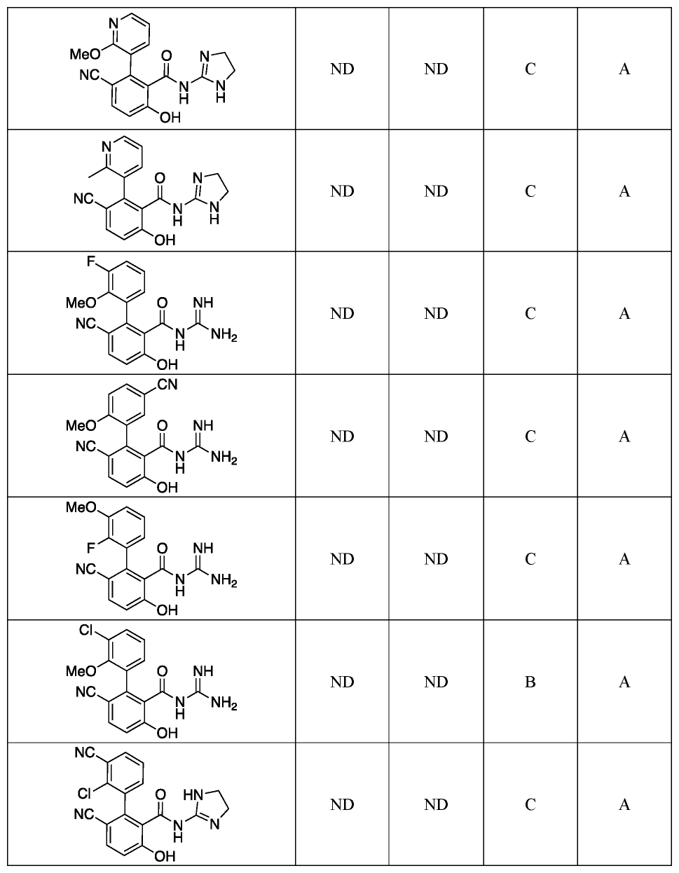

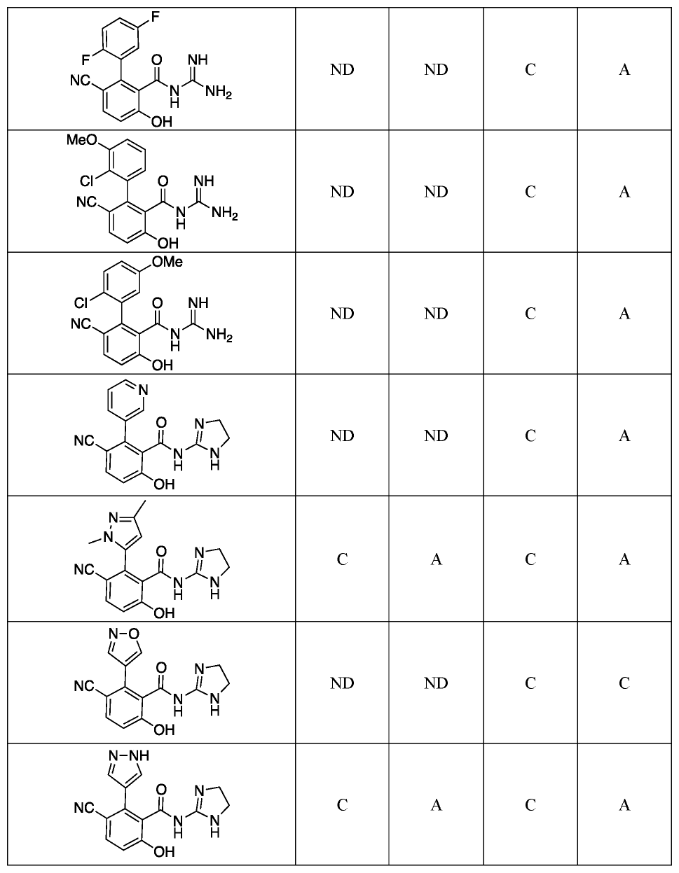

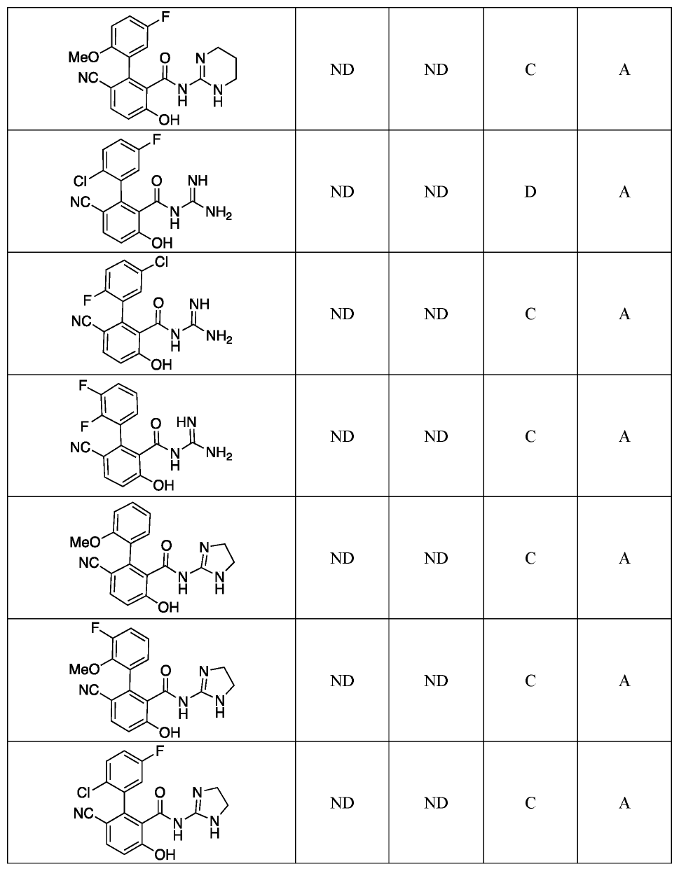

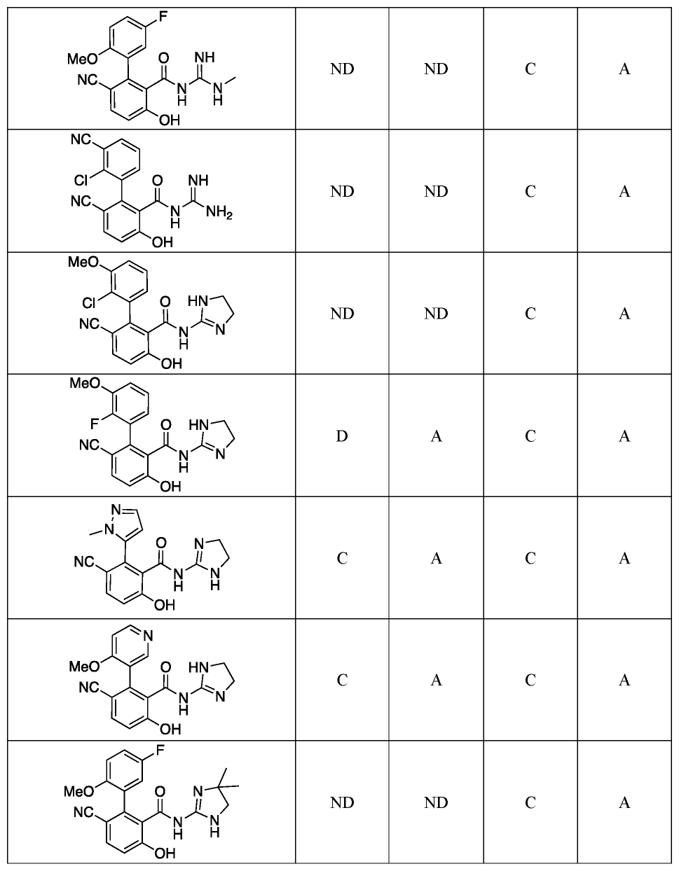

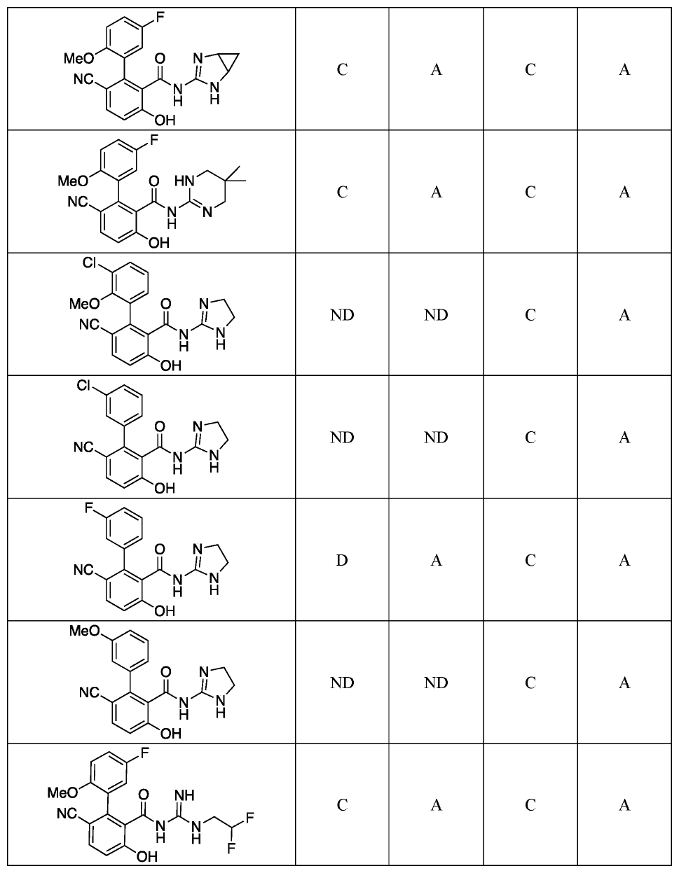

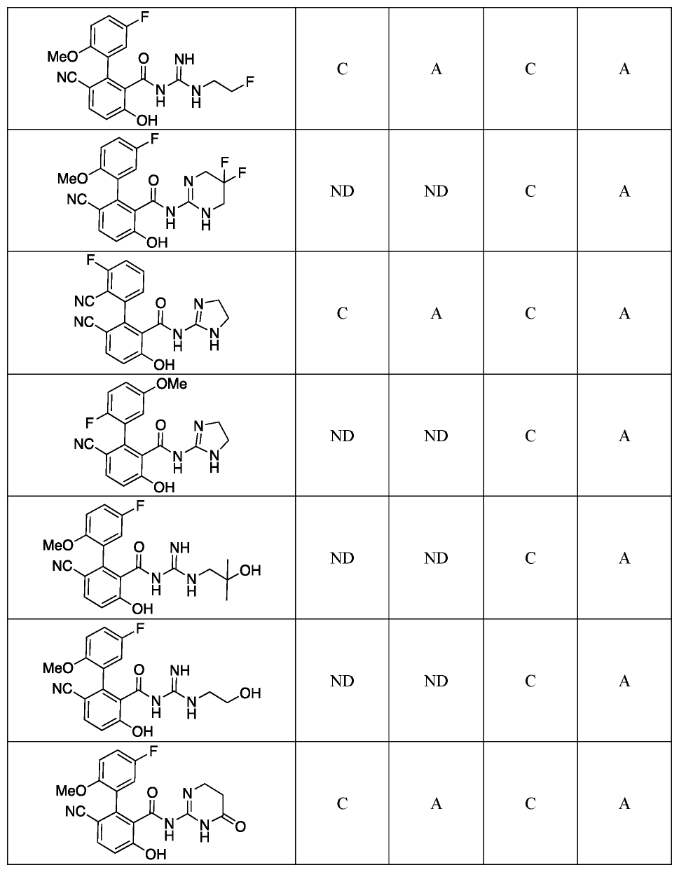

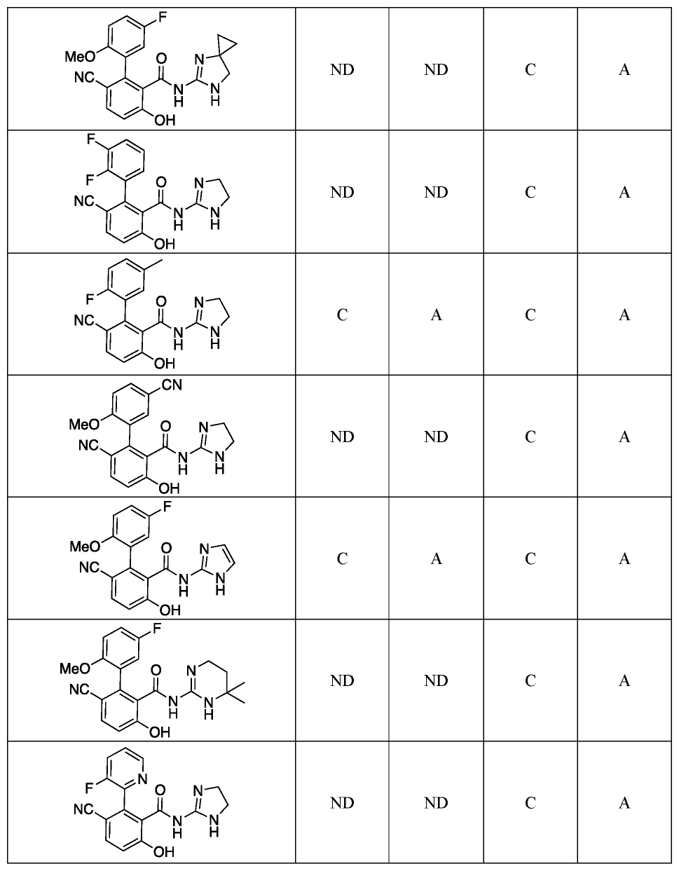

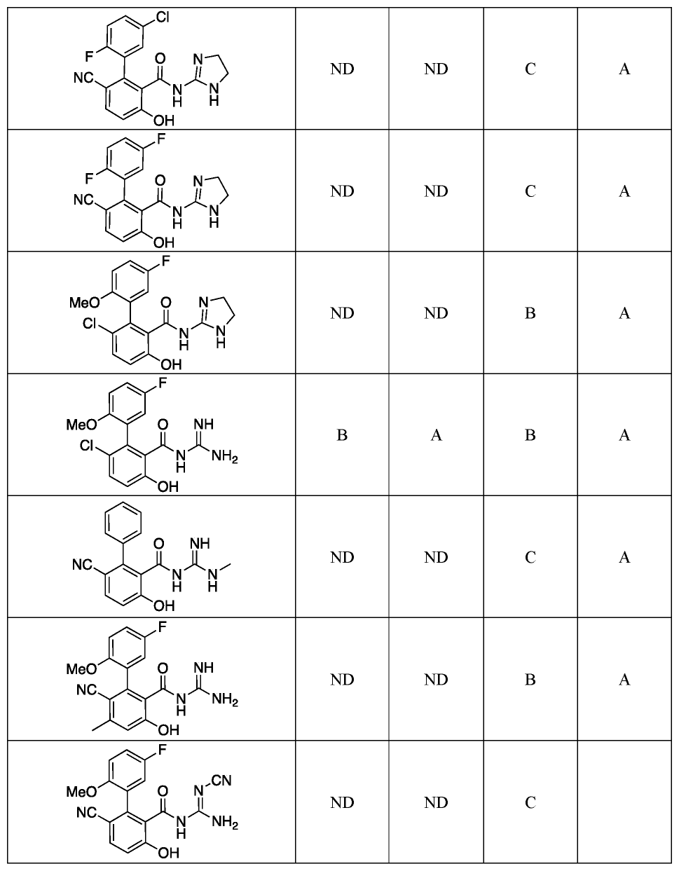

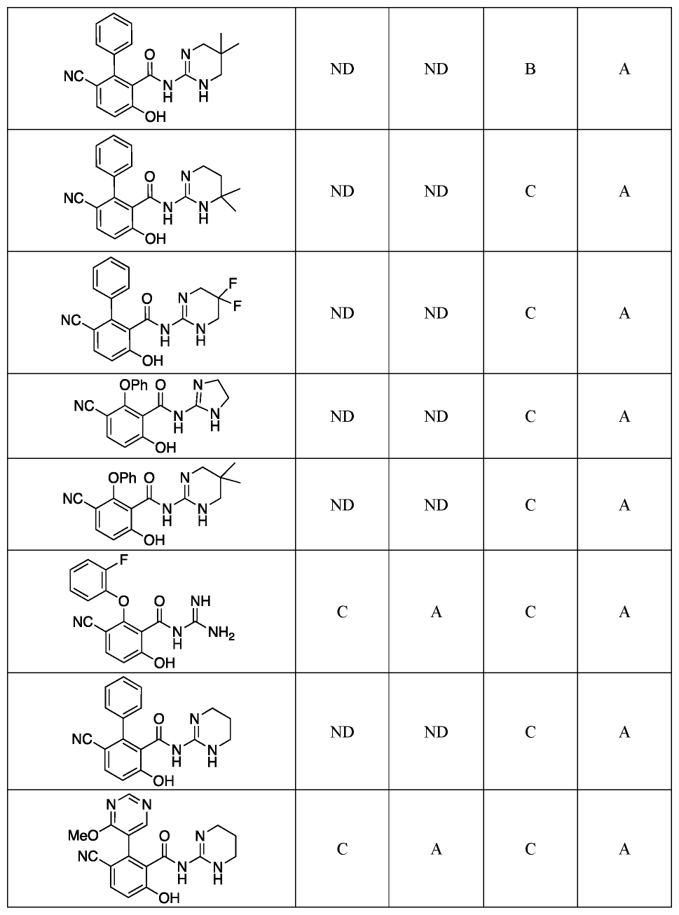

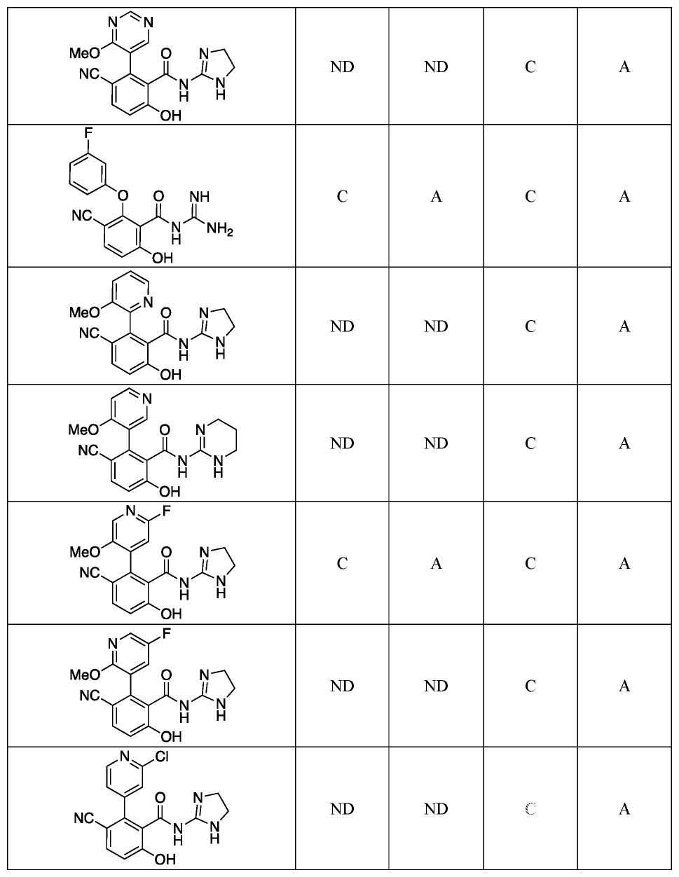

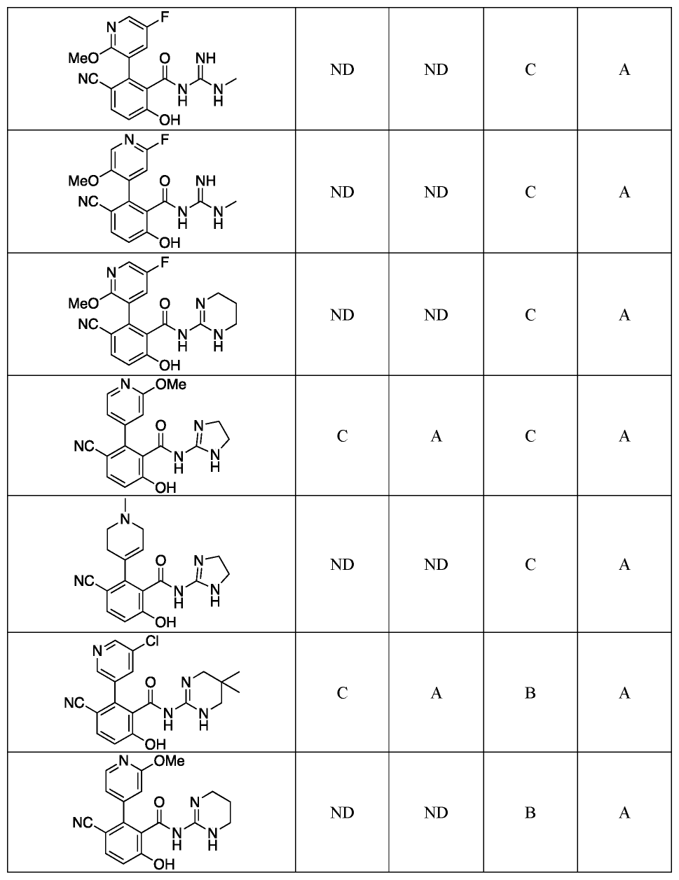

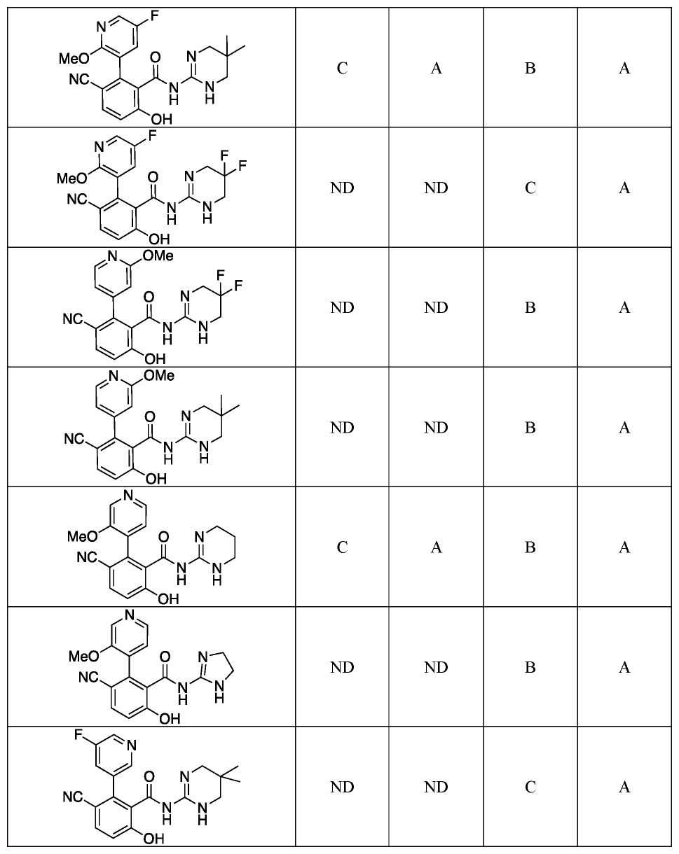

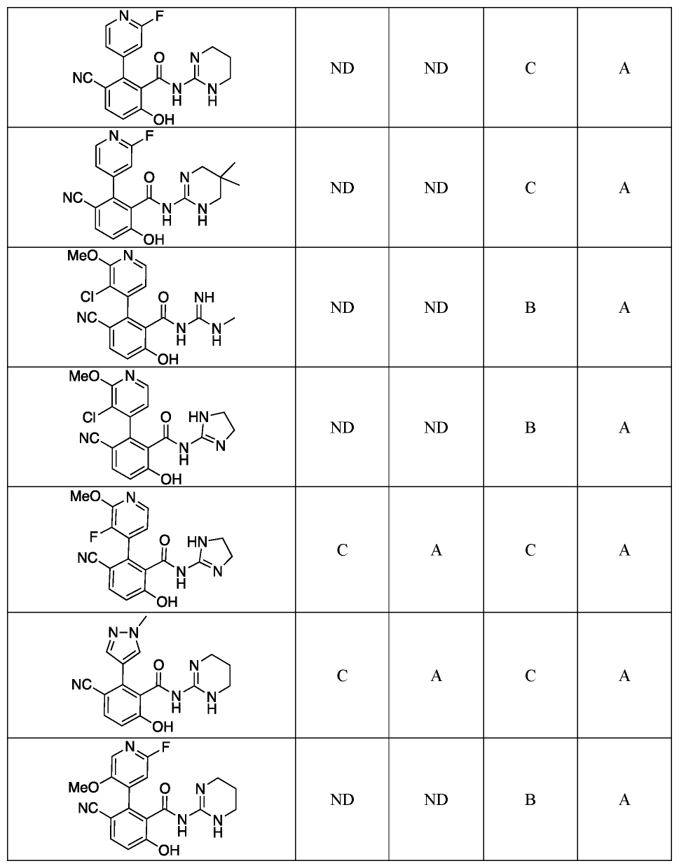

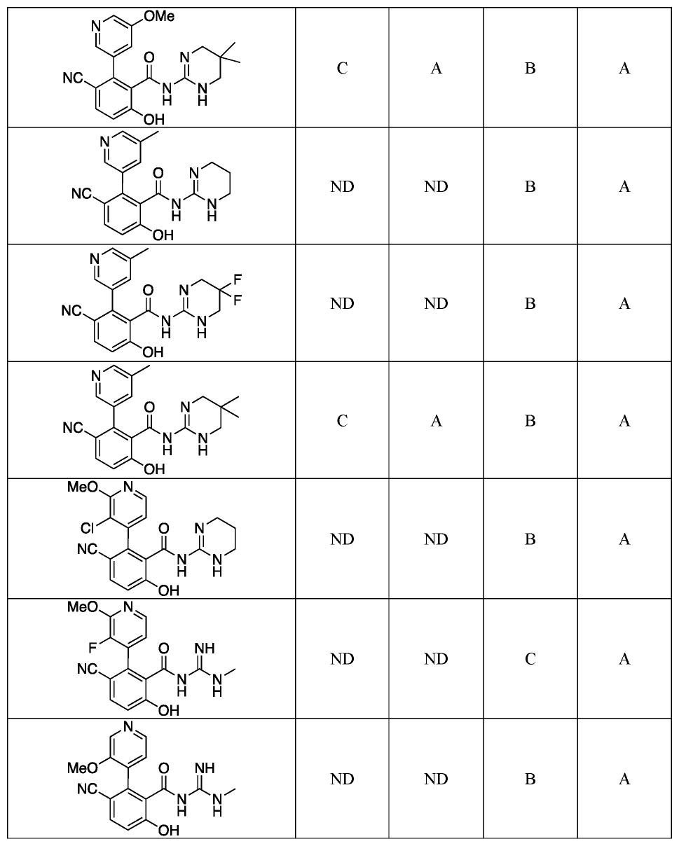

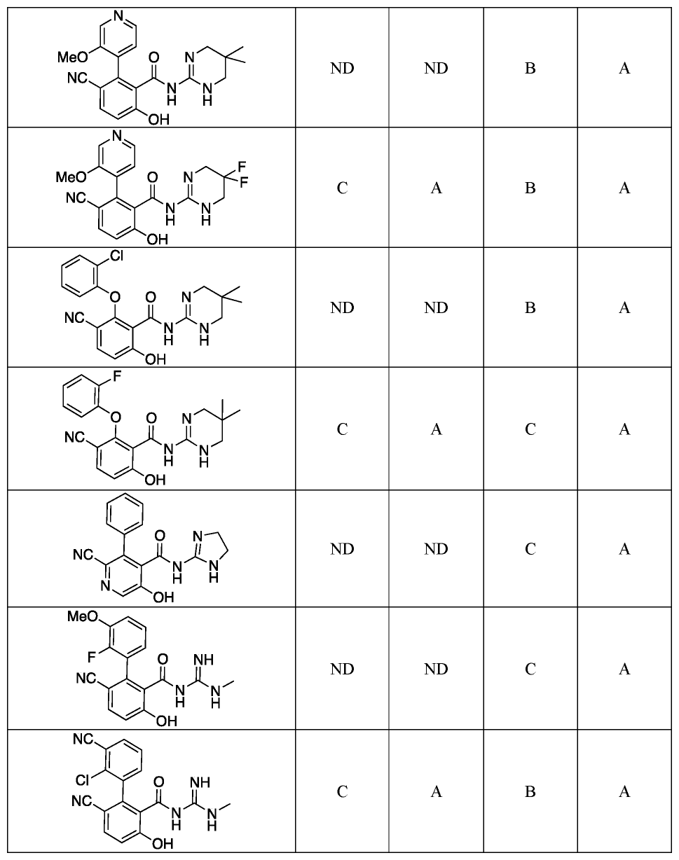

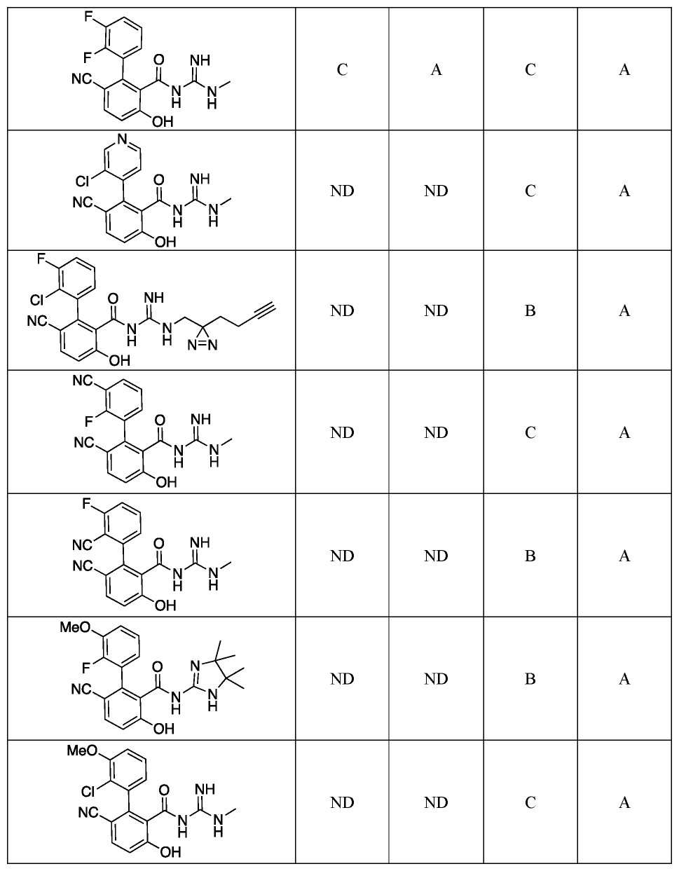

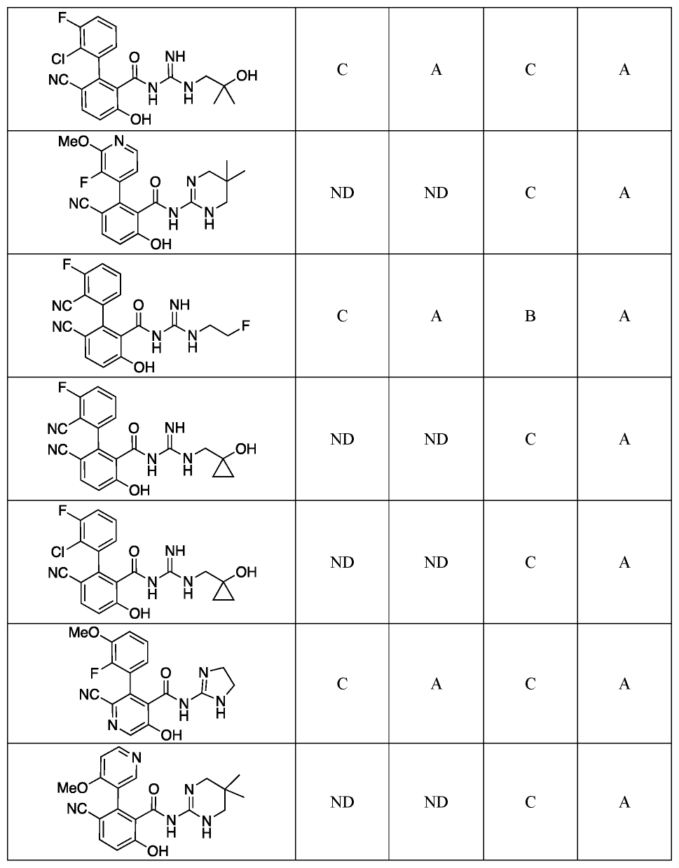

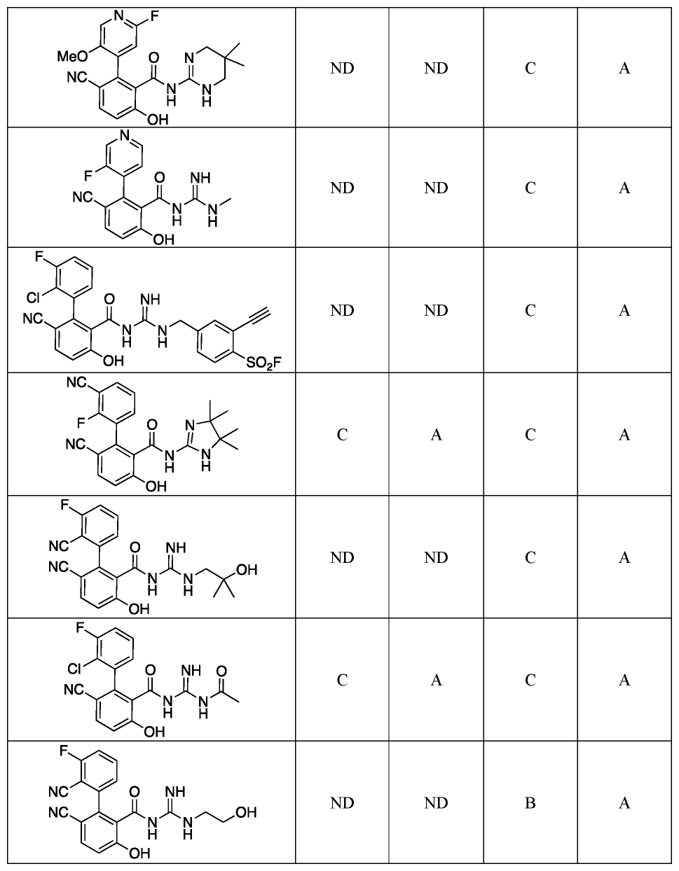

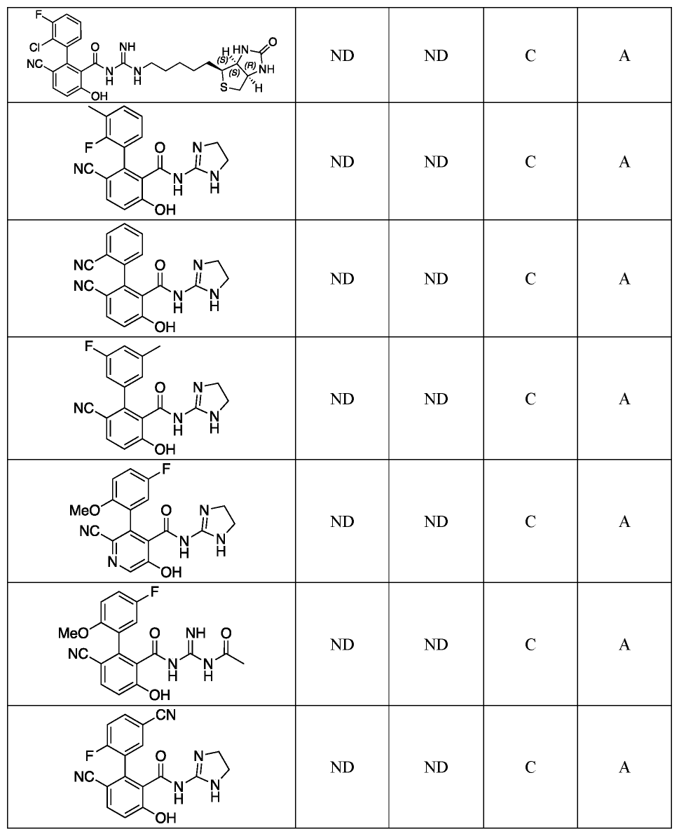

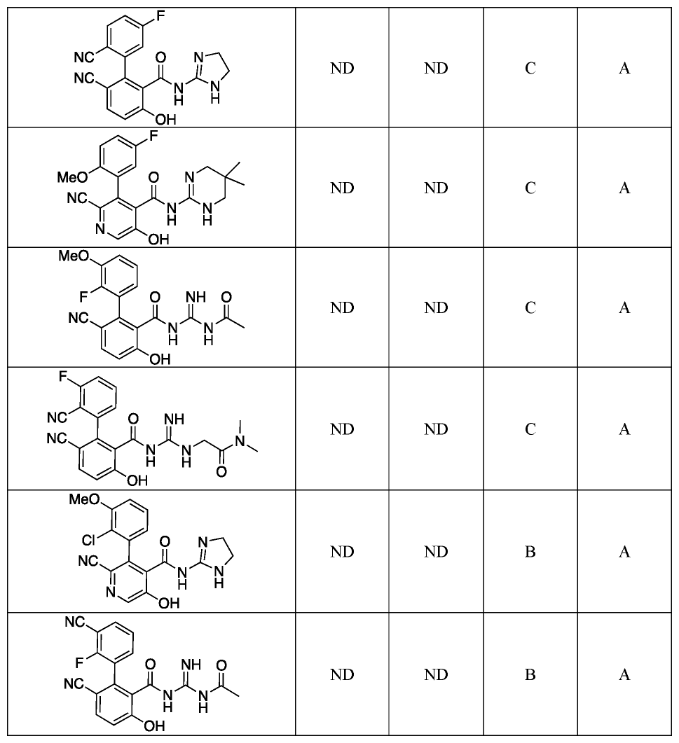

### DETAILED DESCRIPTION

### Definitions

For convenience, before further description of the present invention, certain terms employed in the specification, examples and appended claims are collected here. These definitions should be read in light of the remainder of the disclosure and understood as by a person of skill in the art. Unless defined otherwise, all technical and scientific terms used herein have the same meaning as commonly understood by a person of ordinary skill in the art.

In order for the present invention to be more readily understood, certain terms and phrases are defined below and throughout the specification.

The articles “a” and “an” are used herein to refer to one or to more than one (i.e., to at least one) of the grammatical object of the article. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Certain compounds contained in compositions of the present invention may exist in particular geometric or stereoisomeric forms. In addition, polymers of the present invention may also be optically active. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

“Geometric isomer” means isomers that differ in the orientation of substituent atoms in relationship to a carbon-carbon double bond, to a cycloalkyl ring, or to a bridged bicyclic system.

Atoms (other than H) on each side of a carbon-carbon double bond may be in an E (substituents are on opposite sides of the carbon-carbon double bond) or Z (substituents are oriented on the same side) configuration. “R,” “S,” “S*,” “R*,” “E,” “Z,” “cis,” and “trans,” indicate configurations relative to the core molecule. Certain of the disclosed compounds may exist in “atropisomeric” forms or as “atropisomers.” Atropisomers are stereoisomers resulting from hindered rotation about single bonds where the steric strain barrier to rotation is high enough to allow for the isolation of the conformers. The compounds of the invention may be prepared as individual isomers by either isomer-specific synthesis or resolved from a mixture of isomers. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide of each of the isomers of an isomeric pair using an optically pure acid, amine or alcohol (followed by chromatographic separation and removal of the chiral auxiliary), or resolving an isomeric mixture of either a starting material or a final product using various well known chromatographic methods.

If, for instance, a particular enantiomer of compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

Diastereomeric purity by weight is the ratio of the weight of one diastereomer or over the weight of all the diastereomers. When the stereochemistry of a disclosed compound is named or depicted by structure, the named or depicted stereoisomer is at least about 60%, about 70%, about 80%, about 90%, about 99% or about 99.9% by weight relative to the other stereoisomers. When a single enantiomer is named or depicted by structure, the depicted or named enantiomer is at least about 60%, about 70%, about 80%, about 90%, about 99% or about 99.9% by weight optically pure. When a single diastereomer is named or depicted by structure, the depicted or named diastereomer is at least about 60%, about 70%, about 80%, about 90%, about 99% or about 99.9% by weight pure. Percent optical purity is the ratio of the weight of the enantiomer or over the weight of the enantiomer plus the weight of its optical isomer.

Percent purity by mole fraction is the ratio of the moles of the enantiomer (or diastereomer) or over the moles of the enantiomer (or diastereomer) plus the moles of its optical isomer. When the stereochemistry of a disclosed compound is named or depicted by structure, the named or depicted stereoisomer is at least about 60%, about 70%, about 80%, about 90%, about 99% or about 99.9% by mole fraction pure relative to the other stereoisomers. When a single enantiomer is named or depicted by structure, the depicted or named enantiomer is at least about 60%, about 70%, about 80%, about 90%, about 99% or about 99.9% by mole fraction pure. When a single diastereomer is named or depicted by structure, the depicted or named diastereomer is at least about 60%, about 70%, about 80%, about 90%, about 99% or about 99.9% by mole fraction pure.

When a disclosed compound is named or depicted by structure without indicating the stereochemistry, and the compound has at least one chiral center, it is to be understood that the name or structure encompasses either enantiomer of the compound free from the corresponding optical isomer, a racemic mixture of the compound or mixtures enriched in one enantiomer relative to its corresponding optical isomer. When a disclosed compound is named or depicted by structure without indicating the stereochemistry and has two or more chiral centers, it is to be understood that the name or structure encompasses a diastereomer free of other diastereomers, a number of diastereomers free from other diastereomeric pairs, mixtures of diastereomers, mixtures of diastereomeric pairs, mixtures of diastereomers in which one diastereomer is enriched relative to the other diastereomer(s) or mixtures of diastereomers in which one or more diastereomer is enriched relative to the other diastereomers. The invention embraces all of these forms.

Structures depicted herein are also meant to include compounds that differ only in the presence of one or more isotopically enriched atoms. For example, compounds produced by the replacement of a hydrogen with deuterium or tritium, or of a carbon with a ¹³C- or ¹⁴C-enriched carbon are within the scope of this invention.

The term “prodrug” as used herein encompasses compounds that, under physiological conditions, are converted into therapeutically active agents. A common method for making a prodrug is to include selected moieties that are hydrolyzed under physiological conditions to reveal the desired molecule. In other embodiments, the prodrug is converted by an enzymatic activity of the host animal.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material, involved in carrying or transporting the subject chemical from one organ or portion of the body, to another organ or portion of the body. Each carrier must be “acceptable” in the sense of being compatible with the other ingredients of the formulation, not injurious to the patient, and substantially non-pyrogenic. Some examples of materials which can serve as pharmaceutically acceptable carriers include: (1) sugars, such as lactose, glucose, and sucrose; (2) starches, such as corn starch and potato starch; (3) cellulose, and its derivatives, such as sodium carboxymethyl cellulose, ethyl cellulose, and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil, and soybean oil; (10) glycols, such as propylene glycol; (11) polyols, such as glycerin, sorbitol, mannitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations. In certain embodiments, pharmaceutical compositions of the present invention are non-pyrogenic, i.e., do not induce significant temperature elevations when administered to a patient.

The term “pharmaceutically acceptable salts” refers to the relatively non-toxic, inorganic and organic acid addition salts of the compound(s). These salts can be prepared in situ during the final isolation and purification of the compound(s), or by separately reacting a purified compound(s) in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, and laurylsulphonate salts, and the like. (See, for example, Berge et al. (1977) “Pharmaceutical Salts”, J. Pharm. Sci. 66:1-19.)

In other cases, the compounds useful in the methods of the present invention may contain one or more acidic functional groups and, thus, are capable of forming pharmaceutically acceptable salts with pharmaceutically acceptable bases. The term “pharmaceutically acceptable salts” in these instances refers to the relatively non-toxic inorganic and organic base addition salts of a compound(s). These salts can likewise be prepared in situ during the final isolation and purification of the compound(s), or by separately reacting the purified compound(s) in its free acid form with a suitable base, such as the hydroxide, carbonate, or bicarbonate of a pharmaceutically acceptable metal cation, with ammonia, or with a pharmaceutically acceptable organic primary, secondary, or tertiary amine. Representative alkali or alkaline earth salts include the lithium, sodium, potassium, calcium, magnesium, and aluminum salts, and the like.

Representative organic amines useful for the formation of base addition salts include ethylamine, diethylamine, ethylenediamine, ethanolamine, diethanolamine, piperazine, and the like (see, for example, Berge et al., supra).

The term “pharmaceutically acceptable cocrystals” refers to solid coformers that do not form formal ionic interactions with the small molecule.

A “therapeutically effective amount” (or “effective amount”) of a compound with respect to use in treatment, refers to an amount of the compound in a preparation which, when administered as part of a desired dosage regimen (to a mammal, preferably a human) alleviates a symptom, ameliorates a condition, or slows the onset of disease conditions according to clinically acceptable standards for the disorder or condition to be treated or the cosmetic purpose, e.g., at a reasonable benefit/risk ratio applicable to any medical treatment.

The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic, (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof).

The term “patient” refers to a mammal in need of a particular treatment. In certain embodiments, a patient is a primate, canine, feline, or equine. In certain embodiments, a patient is a human.

An aliphatic chain comprises the classes of alkyl, alkenyl and alkynyl defined below. A straight aliphatic chain is limited to unbranched carbon chain moieties. As used herein, the term “aliphatic group” refers to a straight chain, branched-chain, or cyclic aliphatic hydrocarbon group and includes saturated and unsaturated aliphatic groups, such as an alkyl group, an alkenyl group, or an alkynyl group.

“Alkyl” refers to a fully saturated cyclic or acyclic, branched or unbranched carbon chain moiety having the number of carbon atoms specified, or up to 30 carbon atoms if no specification is made. For example, alkyl of 1 to 8 carbon atoms refers to moieties such as methyl, ethyl, propyl, butyl, pentyl, hexyl, heptyl, and octyl, and those moieties which are positional isomers of these moieties. Alkyl of 10 to 30 carbon atoms includes decyl, undecyl, dodecyl, tridecyl, tetradecyl, pentadecyl, hexadecyl, heptadecyl, octadecyl, nonadecyl, eicosyl, heneicosyl, docosyl, tricosyl and tetracosyl. In certain embodiments, a straight chain or branched chain alkyl has 30 or fewer carbon atoms in its backbone (e.g., C₁-C₃₀ for straight chains, C₃-C₃₀ for branched chains), and more preferably 20 or fewer. Alkyl groups may be substituted or unsubstituted.

As used herein, the term “alkylene” refers to an alkyl group having the specified number of carbons, for example from 2 to 12 carbon atoms, that contains two points of attachment to the rest of the compound on its longest carbon chain. Non-limiting examples of alkylene groups include methylene —(CH₂)—, ethylene —(CH₂CH₂)—, n-propylene —(CH₂CH₂CH₂)—, isopropylene —(CH₂CH(CH₃))—, and the like. Alkylene groups can be cyclic or acyclic, branched or unbranched carbon chain moiety, and may be optionally substituted with one or more substituents.

“Cycloalkyl” means mono- or bicyclic or bridged or spirocyclic, or polycyclic saturated carbocyclic rings, each having from 3 to 12 carbon atoms. Likewise, preferred cycloalkyls have from 3-10 carbon atoms in their ring structure, and more preferably have 3-6 carbons in the ring structure. Cycloalkyl groups may be substituted or unsubstituted.

Unless the number of carbons is otherwise specified, “lower alkyl,” as used herein, means an alkyl group, as defined above, but having from one to ten carbons, more preferably from one to six carbon atoms in its backbone structure such as methyl, ethyl, n-propyl, isopropyl, n-butyl, isobutyl, sec-butyl, and tert-butyl. Likewise, “lower alkenyl” and “lower alkynyl” have similar chain lengths. Throughout the application, preferred alkyl groups are lower alkyls. In certain embodiments, a substituent designated herein as alkyl is a lower alkyl.

“Alkenyl” refers to any cyclic or acyclic, branched or unbranched unsaturated carbon chain moiety having the number of carbon atoms specified, or up to 26 carbon atoms if no limitation on the number of carbon atoms is specified; and having one or more double bonds in the moiety. Alkenyl of 6 to 26 carbon atoms is exemplified by hexenyl, heptenyl, octenyl, nonenyl, decenyl, undecenyl, dodenyl, tridecenyl, tetradecenyl, pentadecenyl, hexadecenyl, heptadecenyl, octadecenyl, nonadecenyl, eicosenyl, heneicosoenyl, docosenyl, tricosenyl, and tetracosenyl, in their various isomeric forms, where the unsaturated bond(s) can be located anywhere in the moiety and can have either the (Z) or the (E) configuration about the double bond(s).

“Alkynyl” refers to hydrocarbyl moieties of the scope of alkenyl, but having one or more triple bonds in the moiety.

The term “alkylthio” refers to an alkyl group, as defined above, having a sulfur moiety attached thereto. In certain embodiments, the “alkylthio” moiety is represented by one of —(S)-alkyl, —(S)-alkenyl, —(S)-alkynyl, and —(S)—(CH₂)ₘ—R¹, wherein m and R¹ are defined below. Representative alkylthio groups include methylthio, ethylthio, and the like. The terms “alkoxyl” or “alkoxy” as used herein refers to an alkyl group, as defined below, having an oxygen moiety attached thereto. Representative alkoxyl groups include methoxy, ethoxy, propoxy, tert-butoxy, and the like. An “ether” is two hydrocarbons covalently linked by an oxygen. Accordingly, the substituent of an alkyl that renders that alkyl an ether is or resembles an alkoxyl, such as can be represented by one of —O-alkyl, —O-alkenyl, —O-alkynyl, —O—(CH₂)ₘ—R₁₀, where m and R₁₀ are described below.

The terms “amine” and “amino” are art-recognized and refer to both unsubstituted and substituted amines, e.g., a moiety that can be represented by the formulae:

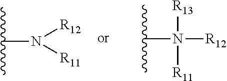

wherein R₁₁, R₁₂ and R₁₃ each independently represent a hydrogen, an alkyl, an alkenyl, —(CH₂)ₘ—R₁₀, or R₁₁ and R₁₂ taken together with the N atom to which they are attached complete a heterocycle having from 4 to 8 atoms in the ring structure; R₁₀ represents an alkenyl, aryl, cycloalkyl, a cycloalkenyl, a heterocyclyl, or a polycyclyl; and m is zero or an integer in the range of 1 to 8. In certain embodiments, only one of R₁₁ or R₁₂ can be a carbonyl, e.g., R₁₁, R₁₂, and the nitrogen together do not form an imide. In even more certain embodiments, R₁₁ and R₁₂ (and optionally R₁₃) each independently represent a hydrogen, an alkyl, an alkenyl, or —(CH₂)ₘ—R₁₀. Thus, the term “alkylamine” as used herein means an amine group, as defined above, having a substituted or unsubstituted alkyl attached thereto, i.e., at least one of R₁₁ and R₁₂ is an alkyl group. In certain embodiments, an amino group or an alkylamine is basic, meaning it has a conjugate acid with a pKₐ>7.00, i.e., the protonated forms of these functional groups have pKₐs relative to water above about 7.00.

The term “amide”, as used herein, refers to a group

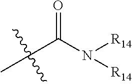

wherein each R₁₄ independently represent a hydrogen or hydrocarbyl group, or two R₁₄ are taken together with the N atom to which they are attached complete a heterocycle having from 4 to 8 atoms in the ring structure.

The term “aryl” as used herein includes 3- to 12-membered substituted or unsubstituted single-ring aromatic groups in which each atom of the ring is carbon (i.e., carbocyclic aryl) or where one or more atoms are heteroatoms (i.e., heteroaryl). Preferably, aryl groups include 5- to 12-membered rings, more preferably 6- to 10-membered rings The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Carboycyclic aryl groups include benzene, naphthalene, phenanthrene, phenol, aniline, and the like. Heteroaryl groups include substituted or unsubstituted aromatic 3- to 12-membered ring structures, more preferably 5- to 12-membered rings, more preferably 5- to 10-membered rings, whose ring structures include one to four heteroatoms. Heteroaryl groups include, for example, pyrrole, furan, thiophene, imidazole, oxazole, thiazole, triazole, pyrazole, pyridine, pyrazine, pyridazine and pyrimidine, and the like. Aryl and heteroaryl can be monocyclic, bicyclic, or polycyclic.

The term “halo”, “halide”, or “halogen” as used herein means halogen and includes, for example, and without being limited thereto, fluoro, chloro, bromo, iodo and the like, in both radioactive and non-radioactive forms. In a preferred embodiment, halo is selected from the group consisting of fluoro, chloro and bromo.

The terms “heterocyclyl” or “heterocyclic group” refer to 3- to 12-membered ring structures, more preferably 5- to 12-membered rings, more preferably 5- to 10-membered rings, whose ring structures include one to four heteroatoms. Heterocycles can be monocyclic, bicyclic, spirocyclic, or polycyclic. Heterocyclyl groups include, for example, thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring can be substituted at one or more positions with such substituents as described above, as for example, halogen, alkyl, aralkyl, alkenyl, alkynyl, cycloalkyl, hydroxyl, amino, nitro, sulfhydryl, imino, amido, phosphate, phosphonate, phosphinate, carbonyl, carboxyl, silyl, sulfamoyl, sulfinyl, ether, alkylthio, sulfonyl, ketone, aldehyde, ester, a heterocyclyl, an aromatic or heteroaromatic moiety, —CF₃, —CN, and the like.

The term “carbonyl” is art-recognized and includes such moieties as can be represented by the formula:

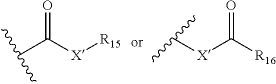

wherein X′ is a bond or represents an oxygen or a sulfur, and R₁₅ represents a hydrogen, an alkyl, an alkenyl, —(CH₂)ₘ—R₁₀ or a pharmaceutically acceptable salt, R₁₆ represents a hydrogen, an alkyl, an alkenyl or —(CH₂)ₘ—R₁₀, where m and R₁₀ are as defined above. Where X′ is an oxygen and R₁₅ or R₁₆ is not hydrogen, the formula represents an “ester.” Where X′ is an oxygen, and R₁₅ is as defined above, the moiety is referred to herein as a carboxyl group, and particularly when R₁₅ is a hydrogen, the formula represents a “carboxylic acid”. Where X′ is an oxygen, and R₁₆ is a hydrogen, the formula represents a “formate.” In general, where the oxygen atom of the above formula is replaced by a sulfur, the formula represents a “thiocarbonyl” group. Where X′ is a sulfur and R₁₅ or R₁₆ is not hydrogen, the formula represents a “thioester” group. Where X′ is a sulfur and R₁₅ is a hydrogen, the formula represents a “thiocarboxylic acid” group. Where X′ is a sulfur and R₁₆ is a hydrogen, the formula represents a “thioformate” group. On the other hand, where X′ is a bond, and R₁₅ is not hydrogen, the above formula represents a “ketone” group. Where X′ is a bond, and R₁₅ is a hydrogen, the above formula represents an “aldehyde” group.

As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc.

As used herein, the term “nitro” means —NO₂; the term “halogen” designates —F, —Cl, —Br, or —I; the term “sulfhydryl” means —SH; the term “hydroxyl” means —OH; the term “sulfonyl” means —SO₂—; the term “azido” means —N₃; the term “cyano” means —CN; the term “isocyanato” means —NCO; the term “thiocyanato” means —SCN; the term “isothiocyanato” means —NCS; and the term “cyanato” means —OCN.

The term “sulfamoyl” is art-recognized and includes a moiety that can be represented by the formula:

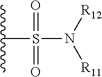

in which R₁₁ and R₁₂ are as defined above.

The term “sulfate” is art recognized and includes a moiety that can be represented by the formula:

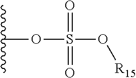

in which R₁₅ is as defined above.

The term “sulfonamide” is art recognized and includes a moiety that can be represented by the formula:

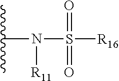

in which R₁₁ and R₁₆ are as defined above.

The term “sulfonate” is art-recognized and includes a moiety that can be represented by the formula:

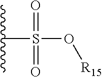

in which R₅₄ is an electron pair, hydrogen, alkyl, cycloalkyl, or aryl.

The terms “sulfoxido” or “sulfinyl”, as used herein, refers to a moiety that can be represented by the formula:

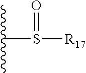

in which R₁₇ is selected from the group consisting of the group consisting of hydrogen, alkyl, alkenyl, alkynyl, cycloalkyl, heterocyclyl, aralkyl, or aryl.

The term “urea” is art-recognized and may be represented by the general formula

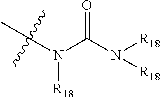

wherein each R₁₈ independently represents hydrogen or a hydrocarbyl, such as alkyl, or any occurrence of R₁₈ taken together with another and the intervening atom(s) complete a heterocycle having from 4 to 8 atoms in the ring structure.

As used herein, the definition of each expression, e.g., alkyl, m, n, etc., when it occurs more than once in any structure, is intended to be independent of its definition elsewhere in the same structure.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxy, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. In preferred embodiments, the substituents on substituted alkyls are selected from C₁₋₆ alkyl, C₃₋₆ cycloalkyl, halogen, carbonyl, cyano, or hydroxyl. In more preferred embodiments, the substituents on substituted alkyls are selected from fluoro, carbonyl, cyano, or hydroxyl. It will be understood by those skilled in the art that substituents can themselves be substituted, if appropriate. Unless specifically stated as “unsubstituted,” references to chemical moieties herein are understood to include substituted variants. For example, reference to an “aryl” group or moiety implicitly includes both substituted and unsubstituted variants.

As used herein, “small molecules” refers to small organic or inorganic molecules of molecular weight below about 3,000 Daltons. In general, small molecules useful for the invention have a molecular weight of less than 3,000 Daltons (Da). The small molecules can be, e.g., from at least about 100 Da to about 3,000 Da (e.g., between about 100 to about 3,000 Da, about 100 to about 2500 Da, about 100 to about 2,000 Da, about 100 to about 1,750 Da, about 100 to about 1,500 Da, about 100 to about 1,250 Da, about 100 to about 1,000 Da, about 100 to about 750 Da, about 100 to about 500 Da, about 200 to about 1500, about 500 to about 1000, about 300 to about 1000 Da, or about 100 to about 250 Da).

In some embodiments, a “small molecule” refers to an organic, inorganic, or organometallic compound typically having a molecular weight of less than about 1000. In some embodiments, a small molecule is an organic compound, with a size on the order of 1 nm. In some embodiments, small molecule drugs of the invention encompass oligopeptides and other biomolecules having a molecular weight of less than about 1000.

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a composition depends on the composition selected. The compositions can be administered from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the compositions described herein can include a single treatment or a series of treatments.

The terms “decrease,” “reduce,” “reduced”, “reduction”, “decrease,” and “inhibit” are all used herein generally to mean a decrease by a statistically significant amount relative to a reference. However, for avoidance of doubt, “reduce,” “reduction” or “decrease” or “inhibit” typically means a decrease by at least 10% as compared to a reference level and can include, for example, a decrease by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, up to and including, for example, the complete absence of the given entity or parameter as compared to the reference level, or any decrease between 10-99% as compared to the absence of a given treatment.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

As used herein, the term “modulate” includes up-regulation and down-regulation, e.g., enhancing or inhibiting a response.

A “radiopharmaceutical agent,” as defined herein, refers to a pharmaceutical agent which contains at least one radiation-emitting radioisotope. Radiopharmaceutical agents are routinely used in nuclear medicine for the diagnosis and/or therapy of various diseases. The radiolabelled pharmaceutical agent, for example, a radiolabelled antibody, contains a radioisotope (RI) which serves as the radiation source. As contemplated herein, the term “radioisotope” includes metallic and non-metallic radioisotopes. The radioisotope is chosen based on the medical application of the radiolabeled pharmaceutical agents. When the radioisotope is a metallic radioisotope, a chelator is typically employed to bind the metallic radioisotope to the rest of the molecule. When the radioisotope is a non-metallic radioisotope, the non-metallic radioisotope is typically linked directly, or via a linker, to the rest of the molecule.

For purposes of this invention, the chemical elements are identified in accordance with the Periodic Table of the Elements, CAS version, Handbook of Chemistry and Physics, 67th Ed., 1986-87, inside cover.

### Compounds of the Invention

One aspect of the invention relates to compound of Formula (I) or (II):

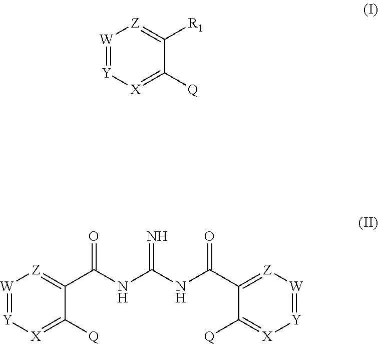

wherein

X, Y, W, and Z are independently selected from N and C(R); provided that no more than two of X, Y, W, and Z are N;

if Z and W, or W and Y, or Y and X are C(R), then any two adjacent instances of R taken together may form a fused 3-8 membered ring;

Q is OH, —NHSO₂R′, —COOH, —C(O)NHSO₂R″, —SO₂NHC(O)R″, tetrazolyl, or —CRₓR_{y}OH;

R is independently selected from H, alkyl, alkenyl, alkynyl, cycloalkyl, heteroalkyl, cycloheteroalkyl, substituted or unsubstituted aryl, substituted or unsubstituted -alkylene-aryl, substituted or unsubstituted heteroaryl, substituted or unsubstituted -alkylene-heteroaryl, haloalkyl, halocycloalkyl, halocycloheteroalkyl, —O-alkyl, —O-haloalkyl, —O-cycloalkyl, —N-alkyl, —N-haloalkyl, —N-cycloalkyl, —S-alkyl, —S-haloalkyl, —S-cycloalkyl, —O-heteroalkyl, —O— cycloheteroalkyl, —N-heteroalkyl, —N-cycloheteroalkyl, —S-heteroalkyl, —S-cycloheteroalkyl, —O-aryl, —N-aryl, —S-aryl, —O-heteroaryl, —N-heteroaryl, —S-heteroaryl, substituted or unsubstituted —O-alkylene-aryl, substituted or unsubstituted —N-alkylene-aryl, substituted or unsubstituted —S-alkylene-aryl, substituted or unsubstituted —O-alkylene-heteroaryl, substituted or unsubstituted —N-alkylene-heteroaryl, substituted or unsubstituted —S-alkylene-heteroaryl, halide, —CN, —NO₂, —S(O)Rₐ, —S(O)₂Rₐ, —C(O)Rₐ, —C(O)₂Rₐ, —C(O)NRₐR_{b}, OH, and C(O)NR′C(NR′)NRₐR_{b};

R′ is H, alkyl, or aryl;

R″ is alkyl or aryl;

Rₐ and R_{b} are independently H, alkyl, alkenyl, alkynyl, substituted or unsubstituted aryl, cycloalkyl, heteroalkyl, haloalkyl, cycloheteroalkyl, halocycloalkyl, halocycloheteroalkyl, substituted or unsubstituted heteroaryl, substituted or unsubstituted -alkylene-aryl, substituted or unsubstituted -alkylene-heteroaryl or Rₐ and R_{b} taken together with the nitrogen atom to which they are attached may form a 3-8 membered ring;

R₁ is

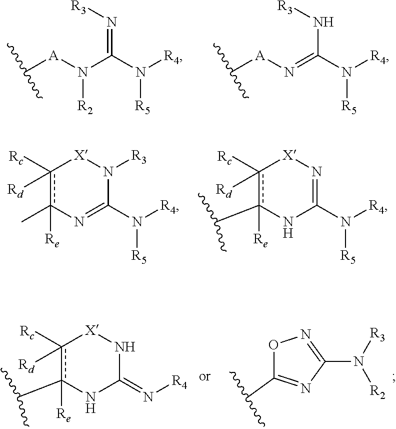

R₂, R₃, R₄, and R₅ are independently selected from H, alkyl, alkenyl, alkynyl, heteroalkyl, cycloalkyl, haloalkyl, halocycloalkyl, cycloheteroalkyl, substituted or unsubstituted aryl, substituted or unsubstituted -alkylene-aryl, substituted or unsubstituted heteroaryl, substituted or unsubstituted -alkylene-heteroaryl, substituted or unsubstituted 5-12 membered ring, alkylenealkoxy, haloalkyl, —CN, —C(O)Rₐ, and —C(O)NRₐR_{b}; provided that (i) no more than one of R₂, R₃, R₄, and R₅ is —CN, (ii) no more than one of R₂, R₃, R₄, and R₅ is —C(O)Rₐ, and (iii) no more than one of R₂, R₃, R₄, and R₅ is —C(O)NRₐR_{b};

R₃ and R₄ taken together may form a 5-8 membered ring;

R₂ and R₅ taken together may form a 5-8 membered ring;

R₄ and R₅ taken together may form a 5-8 membered ring;

R_{c} is H, or alkyl;

R_{d} and Rₑ are independently absent, H, or alkyl;

Rₓ and R_{y} are independently H, F, alkyl, aryl, or haloalkyl;

[symbol] represents a single bond or a double bond; if [symbol] is a double bond, then R_{d} and Rₑ are absent;

A is absent, —CH₂—, —C(O)—, —C(S)—, —S(O)₂—, or —CR_{f}R_{g}—;

X′ is absent, —CH₂—, —C(O)—, —C(S)—, or —S(O)₂—; and

R_{f} and R_{g} are independently selected from H, alkyl, alkenyl, alkynyl substituted or unsubstituted aryl, substituted or unsubstituted heteroaryl, substituted or unsubstituted -alkylene-aryl, substituted or unsubstituted -alkylene-heteroaryl, and halide; or R_{f} and R_{g} taken together may form a spirocyclic 3-8 membered ring, or heterospirocyclic 3-8 membered ring.

In some embodiments, X is N. In some embodiments, X is C(R).

In some embodiments, Y is N. In some embodiments, Y is C(R).

In some embodiments, W is N. In some embodiments, W is C(R).

In some embodiments, Z is N. In some embodiments, Z is C(R).

In some embodiments, X, Y, W, and Z are C(R).

In some embodiments, R is H. In some embodiments, R is alkyl. In some embodiments, R is methyl, ethyl, iso-propyl, n-propyl, n-butyl, iso-butyl, sec-butyl, or t-butyl. In some embodiments, R is alkenyl. In some embodiments, R is alkynyl. In some embodiments, R is cycloalkyl. In some embodiments, R is cyclopropyl, cyclobutyl, cyclopentyl, or cyclohexyl. In some embodiments, R is heteroalkyl. In some embodiments, R is cycloheteroalkyl. In some embodiments, R is substituted aryl. In some embodiments, R is unsubstituted aryl. In some embodiments, aryl is phenyl. In some embodiments, R is substituted -alkylene-aryl. In some embodiments, R is unsubstituted -alkylene-aryl. In some embodiments, alkylene is methylene. In some embodiments, R is substituted heteroaryl. In some embodiments, R is unsubstituted heteroaryl. In some embodiments, R is substituted -alkylene-heteroaryl. In some embodiments, R is unsubstituted -alkylene-heteroaryl. In some embodiments, R is —O-alkyl. In some embodiments, R is —N-alkyl. In some embodiments, R is —S-alkyl. In some embodiments, alkyl is methyl, ethyl, iso-propyl, n-propyl, n-butyl, iso-butyl, sec-butyl, or t-butyl. In some embodiments, R is —O-cycloalkyl. In some embodiments, R is —N-cycloalkyl. In some embodiments, R is —S-cycloalkyl. In some embodiments, R is —O-haloalkyl. In some embodiments, R is —N-haloalkyl. In some embodiments, R is —S-haloalkyl. In some embodiments, R is halocycloalkyl. In some embodiments, R is halocycloheteroalkyl. In some embodiments, R is —O-heteroalkyl. In some embodiments, R is —N-heteroalkyl. In some embodiments, R is —S-heteroalkyl. In some embodiments, R is —O-cycloheteroalkyl. In some embodiments, R is —N-cycloheteroalkyl. In some embodiments, R is —S-cycloheteroalkyl. In some embodiments, heteroalkyl is unsubstituted heteroalkyl. In some embodiments, heteroalkyl is substituted heteroalkyl. In some embodiments, R is —O-aryl. In some embodiments, R is —N-aryl. In some embodiments, R is —S-aryl. In some embodiments, aryl is unsubstituted aryl. In some embodiments, aryl is substituted aryl. In some embodiments, aryl is phenyl. In some embodiments, R is —O-heteroaryl. In some embodiments, R is —N-heteroaryl. In some embodiments, R is —S-heteroaryl. In some embodiments, heteroaryl is substituted heteroaryl. In some embodiments, heteroaryl is unsubstituted heteroaryl.

In some embodiments, R is unsubstituted —O-alkylene-aryl. In some embodiments, R is substituted —O-alkylene-aryl. In some embodiments, R is unsubstituted —N-alkylene-aryl. In some embodiments, R is substituted —N-alkylene-aryl. In some embodiments, R is unsubstituted —S-alkylene-aryl. In some embodiments, R is substituted —S-alkylene-aryl. In some embodiments, R is unsubstituted —O-alkylene-heteroaryl. In some embodiments, R is substituted —O-alkylene-heteroaryl. In some embodiments, R is unsubstituted —N-alkylene-heteroaryl. In some embodiments, R is substituted —N-alkylene-heteroaryl. In some embodiments, R is unsubstituted —S-alkylene-heteroaryl. In some embodiments, R is substituted —S-alkylene-heteroaryl.

In some embodiments, R is halide. In some embodiments, R is Cl, F, or Br. In some embodiments, R is haloalkyl. In some embodiments, haloalkyl is —C(H)F₂. In some embodiments, R is —O-haloalkyl. In some embodiments, —O-haloalkyl is —OCF₃. In some embodiments, R is —N-haloalkyl. In some embodiments, —N-haloalkyl is —NCH₂CF₃. In some embodiments, R is —CN. In some embodiments, R is —S(O)Rₐ. In some embodiments, R is —S(O)₂Rₐ. In some embodiments, R is —C(O)Rₐ. In some embodiments, is —C(O)₂Rₐ. In some embodiments, R is —C(O)NRₐR_{b}. In some embodiments, R is OH. In some embodiments, R is —C(O)NR′C(NR′)NRₐR_{b}

In some embodiments, Rₐ is H. In some embodiments, Rₐ is alkyl. In some embodiments, alkyl is methyl, ethyl, iso-propyl, n-propyl, n-butyl, iso-butyl, sec-butyl, or t-butyl. In some embodiments, Rₐ is alkenyl. In some embodiments, Rₐ is alkynyl. In some embodiments, Rₐ is aryl. In some embodiments, aryl is unsubstituted aryl. In some embodiments, aryl is substituted aryl. In some embodiments, aryl is phenyl. In some embodiments, Rₐ is cycloalkyl. In some embodiments, Rₐ is heteroalkyl. In some embodiments, Rₐ is haloalkyl. In some embodiments, Rₐ is cycloheteroalkyl. In some embodiments, Rₐ is halocycloalkyl. In some embodiments, Rₐ is halocycloheteroalkyl. In some embodiments, Rₐ is unsubstituted -alkylene-aryl. In some embodiments, Rₐ is unsubstituted -alkylene-aryl. In some embodiments, Rₐ is unsubstituted -alkylene-heteroaryl. In some embodiments, Rₐ is unsubstituted -alkylene-heteroaryl. In some embodiments, Rₐ is substituted -alkylene-heteroaryl. In some embodiments, Rₐ is unsubstituted heteroaryl. In some embodiments, Rₐ is substituted heteroaryl.

In some embodiments, R_{b} is H. In some embodiments, R_{b} is alkyl. In some embodiments, alkyl is methyl, ethyl, iso-propyl, n-propyl, n-butyl, iso-butyl, sec-butyl, or t-butyl. In some embodiments, R_{b} is alkenyl. In some embodiments, R_{b} is alkynyl. In some embodiments, R_{b} is aryl. In some embodiments, aryl is unsubstituted aryl. In some embodiments, aryl is substituted aryl. In some embodiments, aryl is phenyl. In some embodiments, R_{b} is cycloalkyl. In some embodiments, R_{b} is heteroalkyl. In some embodiments, R_{b} is haloalkyl. In some embodiments, R_{b} is cycloheteroalkyl. In some embodiments, R_{b} is halocycloalkyl. In some embodiments, R_{b} is halocycloheteroalkyl. In some embodiments, R_{b} is unsubstituted -alkylene-aryl. In some embodiments, R_{b} is unsubstituted -alkylene-aryl. In some embodiments, R_{b} is unsubstituted -alkylene-heteroaryl. In some embodiments, R_{b} is unsubstituted -alkylene-heteroaryl. In some embodiments, R_{b} is substituted -alkylene-heteroaryl. In some embodiments, R_{b} is unsubstituted heteroaryl. In some embodiments, R_{b} is substituted heteroaryl.

In some embodiments, Rₐ and R_{b} can be taken together to form a 3-8 membered ring.

In some embodiments, R₁ is

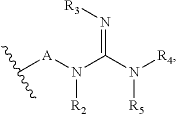

In some embodiments, R₁ is

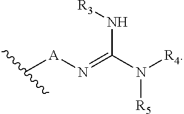

In some embodiments, R₁ is

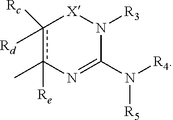

In some embodiments, R₁ is

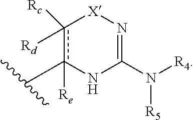

In some embodiments, R₁ is

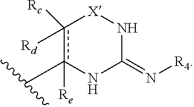

In some embodiments, R₁ is

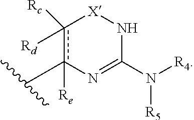

In some embodiments, R₁ is

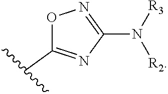

In some embodiments, R₂ is H. In some embodiments, R₂ is alkyl. In some embodiments, R₂ is heteroalkyl. In some embodiments, R₂ is cycloalkyl. In some embodiments, R₂ is cycloheteroalkyl. In some embodiments, R₂ is haloalkyl. In some embodiments, R₂ is halocycloalkyl. In some embodiments, R₂ is substituted aryl. In some embodiments, R₂ is unsubstituted aryl. In some embodiments, R₂ is substituted -alkylene-aryl. In some embodiments, R₂ is unsubstituted -alkylene-aryl. In some embodiments, R₂ is substituted heteroaryl. In some embodiments, R₂ is unsubstituted heteroaryl. In some embodiments, R₂ is substituted -alkylene-heteroaryl. In some embodiments, R₂ is unsubstituted -alkylene-heteroaryl. In some embodiments, R₂ is —C(O)Rₐ. In some embodiments, R₂ is —C(O)NRₐR_{b}. In some embodiments, R₂ is —CN. In some embodiments, R₂ is unsubstituted 5-12 membered ring. In some embodiments, R₂ is substituted 5-12 membered ring. In some embodiments, R₂ is alkylenealkoxy. In some embodiments, R₂ is haloalkyl.

In some embodiments, R₃ is H. In some embodiments, R₃ is alkyl. In some embodiments, R₃ is heteroalkyl. In some embodiments, R₃ is cycloalkyl. In some embodiments, R₃ is cycloheteroalkyl. In some embodiments, R₃ is haloalkyl. In some embodiments, R₃ is halocycloalkyl. In some embodiments, R₃ is substituted aryl. In some embodiments, R₃ is unsubstituted aryl. In some embodiments, R₃ is substituted -alkylene-aryl. In some embodiments, R₃ is unsubstituted -alkylene-aryl. In some embodiments, R₃ is substituted heteroaryl. In some embodiments, R₃ is unsubstituted heteroaryl. In some embodiments, R₃ is substituted -alkylene-heteroaryl. In some embodiments, R₃ is unsubstituted -alkylene-heteroaryl. In some embodiments, R₃ is —C(O)Rₐ. In some embodiments, R₃ is —C(O)NRₐR_{b}. In some embodiments, R₃ is —CN. In some embodiments, R₃ is unsubstituted 5-12 membered ring. In some embodiments, R₃ is substituted 5-12 membered ring. In some embodiments, R₃ is alkylenealkoxy. In some embodiments, R₃ is haloalkyl.

In some embodiments, R₄ is H. In some embodiments, R₄ is alkyl. In some embodiments, R₄ is heteroalkyl. In some embodiments, R₄ is cycloalkyl. In some embodiments, R₄ is cycloheteroalkyl. In some embodiments, R₄ is haloalkyl. In some embodiments, R₄ is halocycloalkyl. In some embodiments, R₄ is substituted aryl. In some embodiments, R₄ is unsubstituted aryl. In some embodiments, R₄ is substituted -alkylene-aryl. In some embodiments, R₄ is unsubstituted -alkylene-aryl. In some embodiments, R₄ is substituted heteroaryl. In some embodiments, R₄ is unsubstituted heteroaryl. In some embodiments, R₄ is substituted -alkylene-heteroaryl. In some embodiments, R₄ is unsubstituted -alkylene-heteroaryl. In some embodiments, R₄ is —C(O)Rₐ. In some embodiments, R₄ is —C(O)NRₐR_{b}. In some embodiments, R₄ is —CN. In some embodiments, R₄ is unsubstituted 5-12 membered ring. In some embodiments, R₄ is substituted 5-12 membered ring. In some embodiments, R₄ is alkylenealkoxy. In some embodiments, R₄ is haloalkyl.

In some embodiments, R₅ is H. In some embodiments, R₅ is alkyl. In some embodiments, R₅ is heteroalkyl. In some embodiments, R₅ is cycloalkyl. In some embodiments, R₅ is cycloheteroalkyl. In some embodiments, R₅ is haloalkyl. In some embodiments, R₅ is halocycloalkyl. In some embodiments, R₅ is substituted aryl. In some embodiments, R₅ is unsubstituted aryl. In some embodiments, R₅ is substituted -alkylene-aryl. In some embodiments, R₅ is unsubstituted -alkylene-aryl. In some embodiments, R₅ is substituted heteroaryl. In some embodiments, R₅ is unsubstituted heteroaryl. In some embodiments, is substituted -alkylene-heteroaryl. In some embodiments, R₅ is unsubstituted -alkylene-heteroaryl. In some embodiments, R₅ is —C(O)Rₐ. In some embodiments, R₅ is —C(O)NRₐR_{b}; In some embodiments, R₅ is —CN. In some embodiments, R₅ is unsubstituted 5-12 membered ring. In some embodiments, R₅ is substituted 5-12 membered ring. In some embodiments, R₅ is alkylenealkoxy. In some embodiments, R₅ is haloalkyl.

In some embodiments, R₂ and R₅ taken together form a 5-8 membered ring.

In some embodiments, R₃ and R₄ taken together form a 5-8 membered ring.

In some embodiments, R₄ and R₅ taken together form a 5-8 membered ring.

In some embodiments, A is absent. In some embodiments, A is —CH₂—. In some embodiments, A is —C(O)—. In some embodiments, A is —C(S)—. In some embodiments, A is —SO₂—. In some embodiments, A is —CR_{f}R_{g}—.

In some embodiments, R_{f} is H. In some embodiments, R_{f} is alkyl. In some embodiments, R_{f} is alkenyl. In some embodiments, R_{f} is alkynyl. In some embodiments, R_{f} is substituted aryl.

In some embodiments, R_{f} is unsubstituted aryl. In some embodiments, R_{f} is substituted heteroaryl. In some embodiments, R_{f} is unsubstituted heteroaryl. In some embodiments, R_{f} is substituted -alkylene-aryl. In some embodiments, R_{f} is unsubstituted -alkylene-aryl. In some embodiments, R_{f} is substituted -alkylene-heteroaryl. In some embodiments, R_{f} is unsubstituted -alkylene-heteroaryl. In some embodiments, R_{f} is halide.

In some embodiments, R_{g} is H. In some embodiments, R_{g} is alkyl. In some embodiments, R_{g} is alkenyl. In some embodiments, R_{g} is alkynyl. In some embodiments, R_{g} is substituted aryl. In some embodiments, R_{g} is unsubstituted aryl. In some embodiments, R_{g} is substituted heteroaryl. In some embodiments, R_{g} is unsubstituted heteroaryl. In some embodiments, R_{g} is substituted -alkylene-aryl. In some embodiments, R_{g} is unsubstituted -alkylene-aryl. In some embodiments, R_{g} is substituted -alkylene-heteroaryl. In some embodiments, R_{g} is unsubstituted -alkylene-heteroaryl. In some embodiments, R_{g} is halide.

In some embodiments, R_{f} and R_{g} taken together form a spirocyclic 3-8 membered ring. In some embodiments, the spirocyclic 3-8 membered ring is a 3-membered spirocyclic ring. In some embodiments, the spirocyclic 3-8 membered ring is a 4-membered spirocyclic ring. In some embodiments, the spirocyclic 3-8 membered ring is a 5-membered spirocyclic ring. In some embodiments, the spirocyclic 3-8 membered ring is a 6-membered spirocyclic ring. In some embodiments, the spirocyclic 3-8 membered ring is a 7-membered spirocyclic ring. In some embodiments, the spirocyclic 3-8 membered ring is a 8-membered spirocyclic ring.

In some embodiments, R_{f} and R_{g} taken together form a heterospirocyclic 3-8 membered ring. In some embodiments, the heterospirocyclic 3-8 membered ring is a 3-membered heterospirocyclic ring. In some embodiments, the heterospirocyclic 3-8 membered ring is a 4-membered heterospirocyclic ring. In some embodiments, the heterospirocyclic 3-8 membered ring is a 5-membered heterospirocyclic ring. In some embodiments, the heterospirocyclic 3-8 membered ring is a 6-membered heterospirocyclic ring. In some embodiments, the heterospirocyclic 3-8 membered ring is a 7-membered heterospirocyclic ring. In some embodiments, the heterospirocyclic 3-8 membered ring is a 8-membered heterospirocyclic ring.

In some embodiments, R_{c} is H. In some embodiments, R_{c} is alkyl.

In some embodiments, R_{d} is H. In some embodiments, R_{d} is alkyl.

In some embodiments, Rₑ is H. In some embodiments, Rₑ is alkyl.

In some embodiments, R_{d} and Rₑ are absent.

In some embodiments, Rₓ is H. In some embodiments, Rₓ is F. In some embodiments, Rₓ is alkyl. In some embodiments, Rₓ is aryl. In some embodiments, Rₓ is haloalkyl.

In some embodiments, R_{y} is H. In some embodiments, R_{y} is F. In some embodiments, R_{y} is alkyl. In some embodiments, R_{y} is aryl. In some embodiments, R_{y} is haloalkyl.

In some embodiments, R′ is H. In some embodiments, R′ is alkyl. In some embodiments, alkyl is methyl. In some embodiments, R′ is aryl.

In some embodiments, R″ is alkyl. In some embodiments, R″ is aryl.

In some embodiments, X′ is absent. In some embodiments, X′ is —CH₂—. In some embodiments, X′ is —C(O)—. In some embodiments, X′ is —C(S)—. In some embodiments, X′ is —S(O)₂—.

In some embodiments, Q is OH. In some embodiments, Q is —NHSO₂R′. In some embodiments, Q is —COOH. In some embodiments, Q is —C(O)NHSO₂R″. In some embodiments, Q is —SO₂NHC(O)R″. In some embodiments, Q is tetrazolyl. In some embodiments, Q is —CRₓR_{y}OH.

In some embodiments, [symbol] represents a single bond.

In some embodiments, [symbol] represents a double bond.

One aspect of the invention relates to compounds of Formula (III):

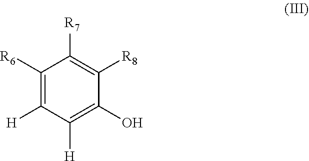

wherein:

R₆ is selected from H, alkyl, cycloalkyl, heteroalkyl, cycloheteroalkyl, substituted or unsubstituted aryl, substituted or unsubstituted -alkylene-aryl, substituted or unsubstituted heteroaryl, substituted or unsubstituted -alkylene-heteroaryl, haloalkyl, halocycloalkyl, halocycloheteroalkyl, —O-alkyl, —O-haloalkyl, —O-cycloalkyl, —N-alkyl, —N-haloalkyl, —S-alkyl, —O-heteroalkyl, —N-heteroalkyl, —S-heteroalkyl, —O-aryl, —N-aryl, —S-aryl, —S-haloalkyl, —S-cycloalkyl, —O-heteroaryl, —O-cycloheteroalkyl, —N-heteroaryl, —N-cycloalkyl, —N-cycloheteroalkyl, —S-cycloheteroalkyl, —S-heteroaryl, halide, —CN, —NO₂, —S(O)Rₐ, —S(O)₂Rₐ, —C(O)Rₐ, —C(O)₂Rₐ, and —C(O)NRₐR_{b};

Rₐ and R_{b} are independently H, alkyl, alkenyl, alkynyl, substituted or unsubstituted aryl, cycloalkyl, heteroalkyl, haloalkyl, cycloheteroalkyl, halocycloalkyl, halocycloheteroalkyl, substituted or unsubstituted heteroaryl, substituted or unsubstituted -alkylene-aryl, substituted or unsubstituted -alkylene-heteroaryl or Rₐ and R_{b} taken together with the nitrogen atom to which they are attached may form a 3-8 membered ring;

R₇ is H, halide, alkyl, or aryl;

R₈ is

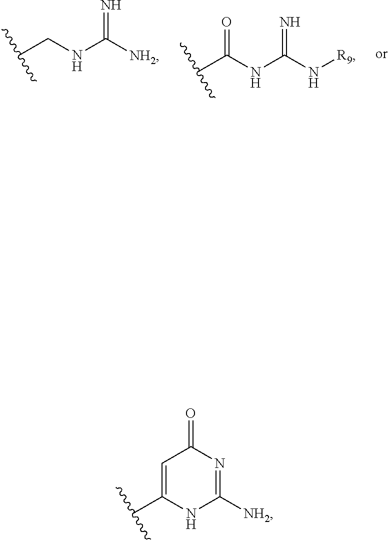

and

R₉ is selected from H, alkyl, alkenyl, alkynyl, heteroalkyl, cycloalkyl, haloalkyl, halocycloalkyl, cycloheteroalkyl, substituted or unsubstituted aryl, substituted or unsubstituted -alkylene-aryl, substituted or unsubstituted heteroaryl, substituted or unsubstituted -alkylene-heteroaryl, —CN, —C(O)Rₐ, and —C(O)NRₐR_{b}.

In some embodiments, R₆ is H. In some embodiments, R₆ is alkyl. In some embodiments, R₆ is cycloalkyl. In some embodiments, R₆ is heteroalkyl. In some embodiments, R₆ is cycloheteroalkyl. In some embodiments, R₆ is substituted aryl. In some embodiments, R₆ is unsubstituted aryl. In some embodiments, R₆ is unsubstituted -alkylene-aryl. In some embodiments, R₆ is substituted -alkylene-aryl. In some embodiments, R₆ is unsubstituted heteroaryl. In some embodiments, R₆ is substituted heteroaryl. In some embodiments, R₆ is unsubstituted -alkylene-heteroaryl. In some embodiments, R₆ is substituted -alkylene-heteroaryl. In some embodiments, R₆ is haloalkyl. In some embodiments, R₆ is halocycloalkyl. In some embodiments, R₆ is halocycloheteroalkyl. In some embodiments, R₆ is —O-alkyl. In some embodiments, R₆ is —O-haloalkyl. In some embodiments, R₆ is —O-cycloalkyl. In some embodiments, R₆ is —N-alkyl. In some embodiments, R₆ is —N-haloalkyl. In some embodiments, R₆ is —S-alkyl. In some embodiments, R₆ is —O-heteroalkyl. In some embodiments, R₆ is —N-heteroalkyl. In some embodiments, R₆ is —S-heteroalkyl. In some embodiments, R₆ is —O-aryl. In some embodiments, R₆ is —N-aryl. In some embodiments, R₆ is —S-aryl. In some embodiments, R₆ is —S-haloalkyl. In some embodiments, R₆ is —S-cycloalkyl. In some embodiments, R₆ is —O-heteroaryl. In some embodiments, R₆ is —O-cycloheteroalkyl. In some embodiments, R₆ is —N-heteroaryl. In some embodiments, R₆ is —N-cycloalkyl. In some embodiments, R₆ is —N-cycloheteroalkyl. In some embodiments, R₆ is —S-cycloheteroalkyl. In some embodiments, R₆ is —S-heteroaryl. In some embodiments, R₆ is halide. In some embodiments, R₆ is Cl, F, or Br. In some embodiments, R₆ is —CN. In some embodiments, R₆ is —CF₃. In some embodiments, R₆ is —OCF₃. In some embodiments, R₆ is —NO₂. In some embodiments, R₆ is —S(O)Rₐ. In some embodiments, R₆ is —S(O)₂Rₐ. In some embodiments, R₆ is —S(O)₂Me. In some embodiments, R₆ is —C(O)Rₐ. —C(O)₂Rₐ. In some embodiments, R₆ is —C(O)NRₐR_{b}. In some embodiments, R₆ is selected from halide, —CN, —CF₃, —OCF₃, —SO₂Me, and —NO₂.

In some embodiments, R₇ is H. In some embodiments, R₇ is halide. In some embodiments, R₇ is R₇ is Cl or F. In some embodiments, R₇ is R₇ is alkyl. In some embodiments, R₇ is R₇ is methyl, ethyl, n-propyl, iso-propyl, n-butyl, iso-butyl, sec-butyl, or t-butyl. In some embodiments, R₇ is R₇ is methyl. In some embodiments, R₇ is R₇ is aryl. In some embodiments, R₇ is R₇ is phenyl.

In some embodiments, R₈ is

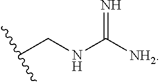 [SMILES: *CNC(=N)N]

In some embodiments, R₈ is

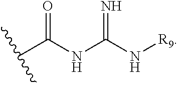

In some embodiments, R₈ is

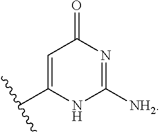 [SMILES: *c1cc(=O)nc(N)[nH]1]

In some embodiments, R₉ is H. In some embodiments, R₄ is alkyl. In some embodiments, R₉ is heteroalkyl. In some embodiments, R₉ is cycloalkyl. In some embodiments, R₉ is cycloheteroalkyl. In some embodiments, R₉ is haloalkyl. In some embodiments, haloalkyl is alkyne-CF₃. In some embodiments, R₉ is alkylene-alkoxy. In some embodiments, alkylene-alkoxy is alkylene-OMe. In some embodiments, R₉ is halocycloalkyl. In some embodiments, R₉ is substituted aryl. In some embodiments, R₉ is unsubstituted aryl. In some embodiments, R₉ is substituted -alkylene-aryl. In some embodiments, R₉ is unsubstituted -alkylene-aryl. In some embodiments, R₉ is substituted heteroaryl. In some embodiments, R₉ is unsubstituted heteroaryl. In some embodiments, R₉ is substituted -alkylene-heteroaryl. In some embodiments, R₉ is unsubstituted -alkylene-heteroaryl. In some embodiments, R₉ is a 5-12 membered ring. In some embodiments, R₉ is —C(O)Rₐ. In some embodiments, R₉ is —C(O)NRₐR_{b}. In some embodiments, R₉ is —CN.

In some embodiments, R₈ is

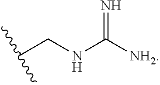 [SMILES: *CNC(=N)N]

In some embodiments, R₈ is

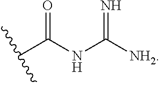 [SMILES: *C(=O)NC(=N)N]

In some embodiments, R₈ is

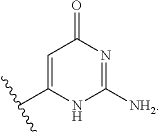 [SMILES: *c1cc(=O)nc(N)[nH]1]

In some embodiments, compound is selected from:

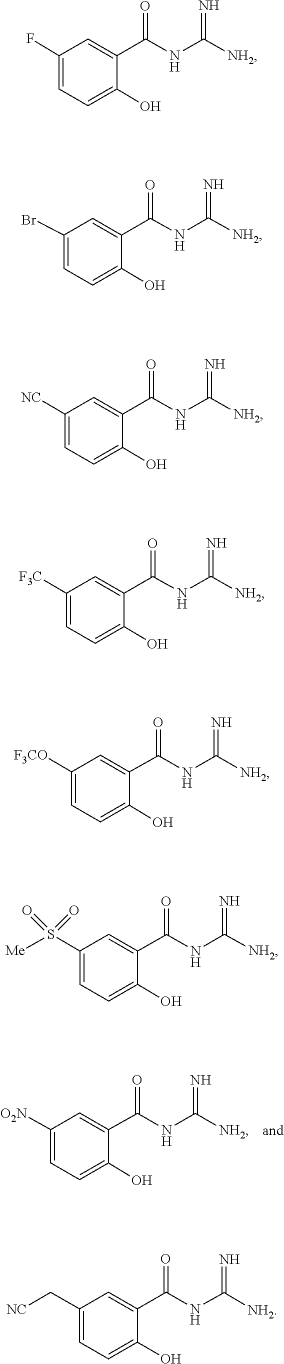

In some embodiments, compound is

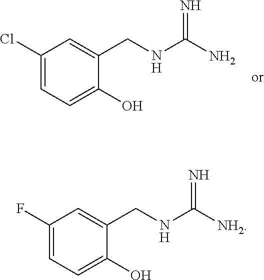 [SMILES: N=C(N)NCc1cc(Cl)ccc1O ; N=C(N)NCc1cc(F)ccc1O]

In some embodiments, the compound is selected from:

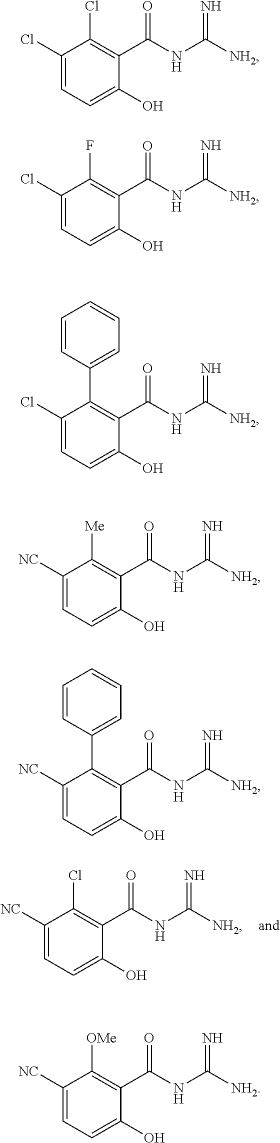

In some embodiments, the compound is selected from:

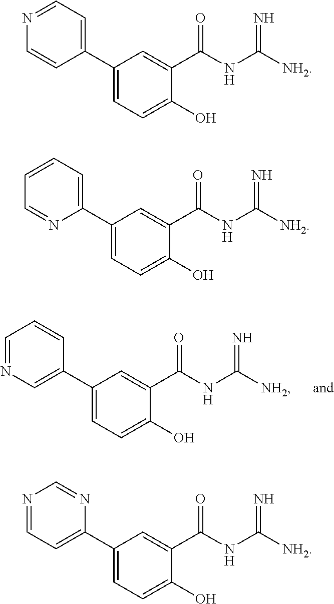 [SMILES: N=C(N)NC(=O)c1cc(-c2ccncc2)ccc1O ; N=C(N)NC(=O)c1cc(-c2ccccn2)ccc1O ; N=C(N)NC(=O)c1cc(-c2cccnc2)ccc1O ; N=C(N)NC(=O)c1cc(-c2ccncn2)ccc1O]

In some embodiments, the compound is

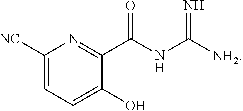 [SMILES: N#Cc1ccc(O)c(C(=O)NC(=N)N)n1]

In some embodiments, the compound is

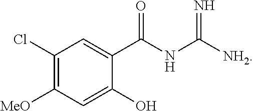 [SMILES: COc1cc(O)c(C(=O)NC(=N)N)cc1Cl]

In some embodiments, the compound is

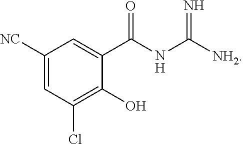 [SMILES: N#Cc1cc(Cl)c(O)c(C(=O)NC(=N)N)c1]

In some embodiments, the compound is selected from

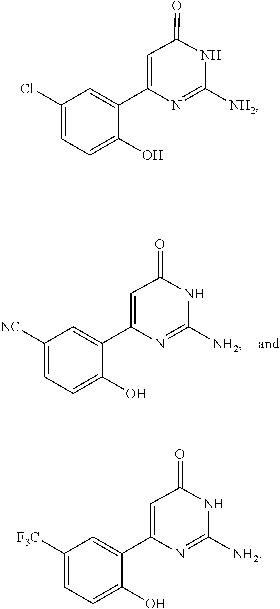 [SMILES: Nc1nc(-c2cc(Cl)ccc2O)cc(=O)[nH]1 ; N#Cc1ccc(O)c(-c2cc(=O)[nH]c(N)n2)c1 ; Nc1nc(-c2cc(C(F)(F)F)ccc2O)cc(=O)[nH]1]

In some embodiments, the compound is selected from

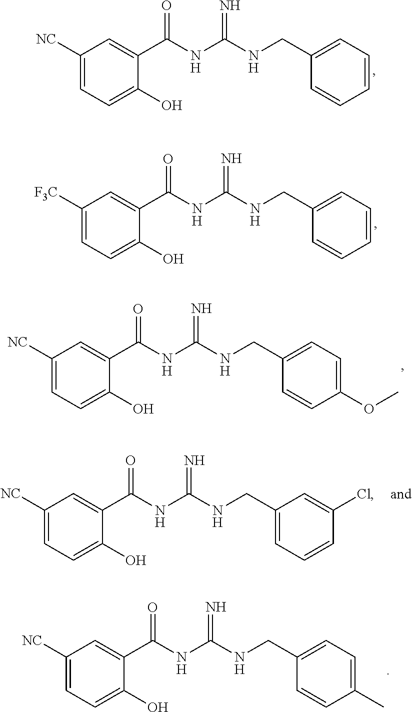 [SMILES: N#Cc1ccc(O)c(C(=O)NC(=N)NCc2ccccc2)c1 ; N=C(NCc1ccccc1)NC(=O)c1cc(C(F)(F)F)ccc1O ; COc1ccc(CNC(=N)NC(=O)c2cc(C#N)ccc2O)cc1 ; N#Cc1ccc(O)c(C(=O)NC(=N)NCc2cccc(Cl)c2)c1 ; Cc1ccc(CNC(=N)NC(=O)c2cc(C#N)ccc2O)cc1]

In some embodiments, the compound is selected from

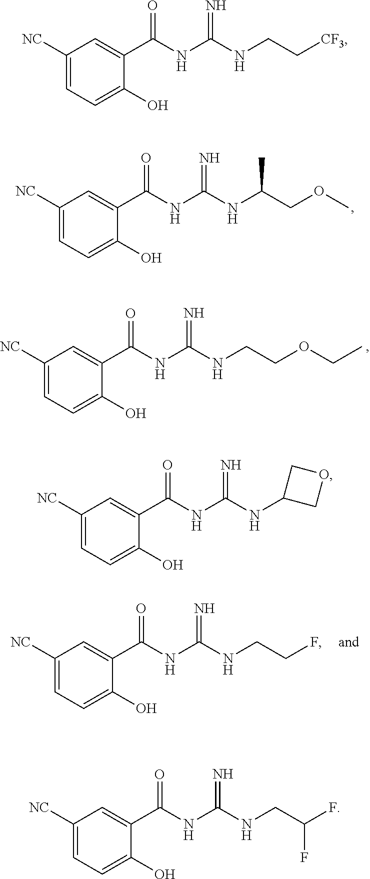 [SMILES: N#Cc1ccc(O)c(C(=O)NC(=N)NCCC(F)(F)F)c1 ; COC[C@H](C)NC(=N)NC(=O)c1cc(C#N)ccc1O ; CCOCCNC(=N)NC(=O)c1cc(C#N)ccc1O ; N#Cc1ccc(O)c(C(=O)NC(=N)NC2COC2)c1 ; N#Cc1ccc(O)c(C(=O)NC(=N)NCCF)c1 ; N#Cc1ccc(O)c(C(=O)NC(=N)NCC(F)F)c1]

In some embodiments, the compound is

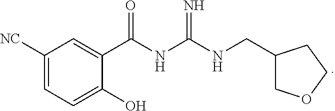 [SMILES: N#Cc1ccc(O)c(C(=O)NC(=N)NCC2CCOC2)c1]

In some embodiments, the compound is selected from

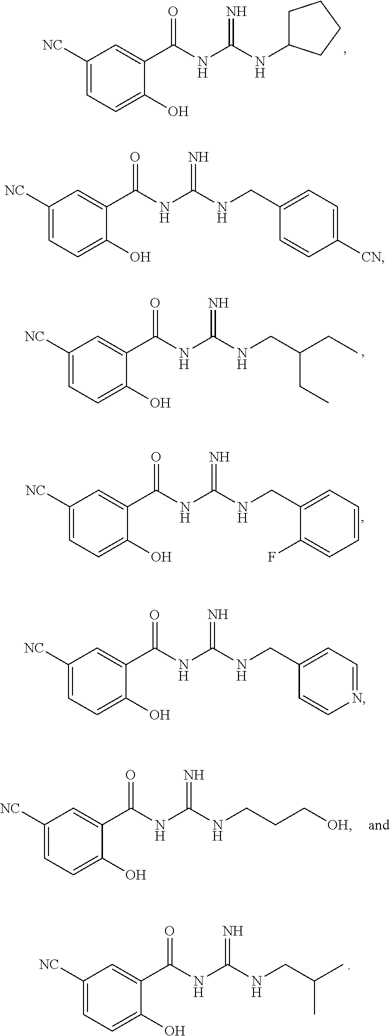

In some embodiments, the compound is selected from

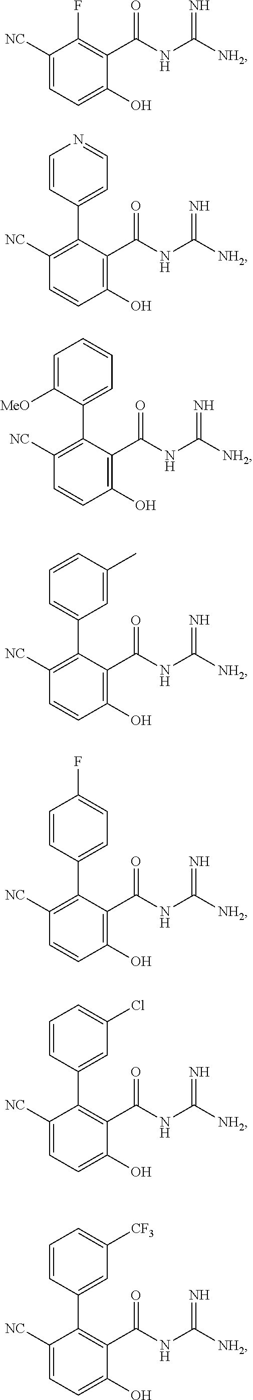

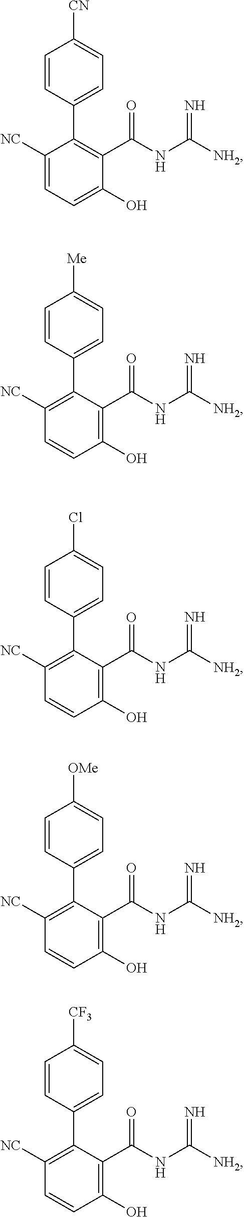

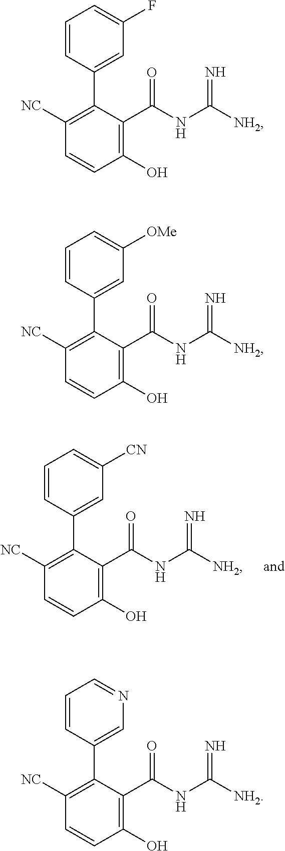 [SMILES: N#Cc1ccc(O)c(C(=O)NC(=N)N)c1-c1cccc(F)c1 ; COc1cccc(-c2c(C#N)ccc(O)c2C(=O)NC(=N)N)c1 ; N#Cc1cccc(-c2c(C#N)ccc(O)c2C(=O)NC(=N)N)c1 ; "and" ; N#Cc1ccc(O)c(C(=O)NC(=N)N)c1-c1cccnc1]

In some embodiments, the compound is

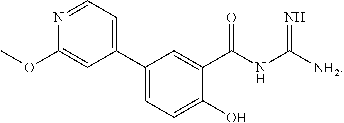 [SMILES: COc1cc(-c2ccc(O)c(C(=O)NC(=N)N)c2)ccn1]

In some embodiments, the compound is selected from the following table:

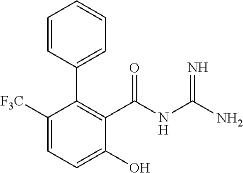 [SMILES: N=C(N)NC(=O)c1c(O)ccc(C(F)(F)F)c1-c1ccccc1]

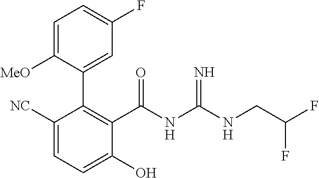 [SMILES: COc1ccc(F)cc1-c1c(C#N)ccc(O)c1C(=O)NC(=N)NCC(F)F]

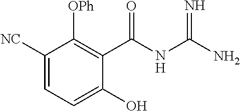 [SMILES: N#Cc1ccc(O)c(C(=O)NC(=N)N)c1Oc1ccccc1]

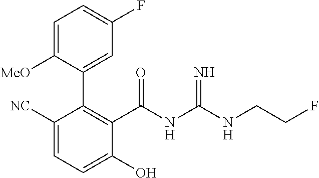 [SMILES: COc1ccc(F)cc1-c1c(C#N)ccc(O)c1C(=O)NC(=N)NCCF]

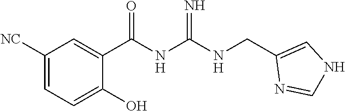 [SMILES: N#Cc1ccc(O)c(C(=O)NC(=N)NCc2c[nH]cn2)c1]

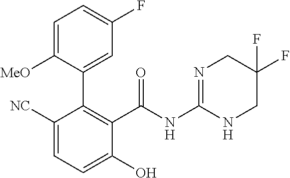 [SMILES: COc1ccc(F)cc1-c1c(C#N)ccc(O)c1C(=O)NC1=NCC(F)(F)CN1]

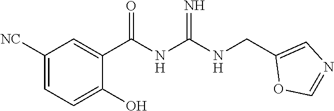 [SMILES: N#Cc1ccc(O)c(C(=O)NC(=N)NCc2cnco2)c1]

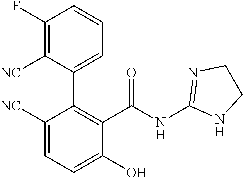 [SMILES: N#Cc1ccc(O)c(C(=O)NC2=NCCN2)c1-c1cccc(F)c1C#N]

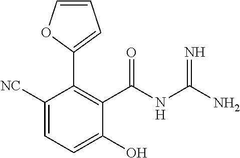 [SMILES: N#Cc1ccc(O)c(C(=O)NC(=N)N)c1-c1ccco1]

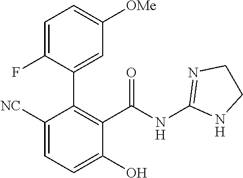 [SMILES: COc1ccc(F)c(-c2c(C#N)ccc(O)c2C(=O)NC2=NCCN2)c1]

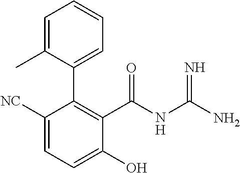 [SMILES: Cc1ccccc1-c1c(C#N)ccc(O)c1C(=O)NC(=N)N]

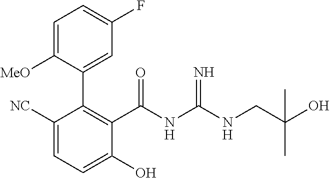 [SMILES: COc1ccc(F)cc1-c1c(C#N)ccc(O)c1C(=O)NC(=N)NCC(C)(C)O]

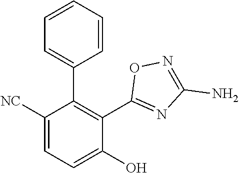 [SMILES: N#Cc1ccc(O)c(-c2nc(N)no2)c1-c1ccccc1]

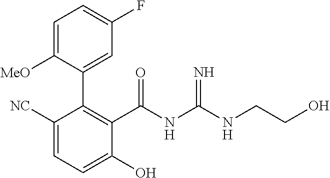 [SMILES: COc1ccc(F)cc1-c1c(C#N)ccc(O)c1C(=O)NC(=N)NCCO]

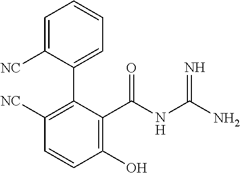 [SMILES: N#Cc1ccccc1-c1c(C#N)ccc(O)c1C(=O)NC(=N)N]

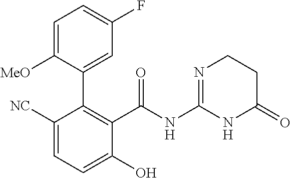 [SMILES: COc1ccc(F)cc1-c1c(C#N)ccc(O)c1C(=O)NC1=NCCC(=O)N1]

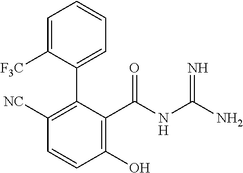 [SMILES: N#Cc1ccc(O)c(C(=O)NC(=N)N)c1-c1ccccc1C(F)(F)F]

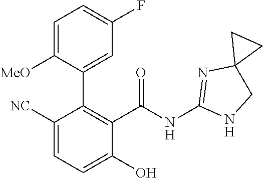 [SMILES: COc1ccc(F)cc1-c1c(C#N)ccc(O)c1C(=O)NC1=NC2(CC2)CN1]

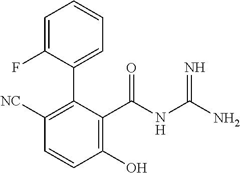 [SMILES: N#Cc1ccc(O)c(C(=O)NC(=N)N)c1-c1ccccc1F]

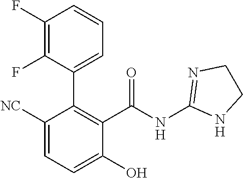 [SMILES: N#Cc1ccc(O)c(C(=O)NC2=NCCN2)c1-c1cccc(F)c1F]

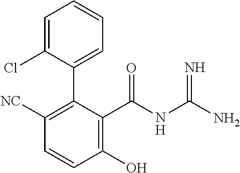 [SMILES: N#Cc1ccc(O)c(C(=O)NC(=N)N)c1-c1ccccc1Cl]

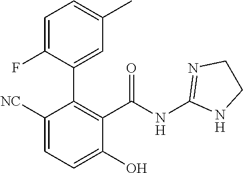 [SMILES: Cc1ccc(F)c(-c2c(C#N)ccc(O)c2C(=O)NC2=NCCN2)c1]

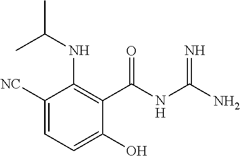 [SMILES: CC(C)Nc1c(C#N)ccc(O)c1C(=O)NC(=N)N]

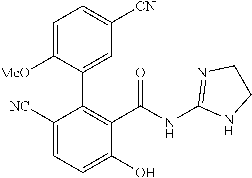 [SMILES: COc1ccc(C#N)cc1-c1c(C#N)ccc(O)c1C(=O)NC1=NCCN1]

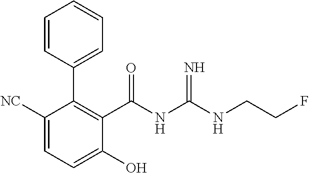 [SMILES: N#Cc1ccc(O)c(C(=O)NC(=N)NCCF)c1-c1ccccc1]

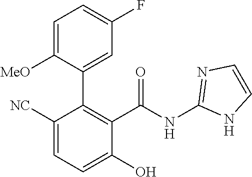 [SMILES: COc1ccc(F)cc1-c1c(C#N)ccc(O)c1C(=O)Nc1ncc[nH]1]

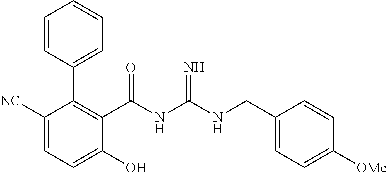 [SMILES: COc1ccc(CNC(=N)NC(=O)c2c(O)ccc(C#N)c2-c2ccccc2)cc1]

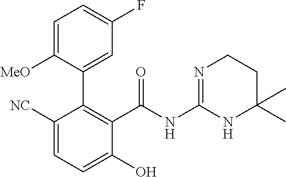 [SMILES: COc1ccc(F)cc1-c1c(C#N)ccc(O)c1C(=O)NC1=NCCC(C)(C)N1]

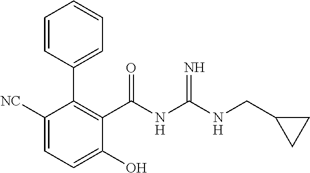 [SMILES: N#Cc1ccc(O)c(C(=O)NC(=N)NCC2CC2)c1-c1ccccc1]

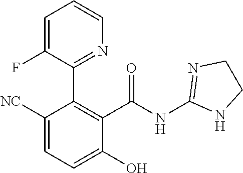 [SMILES: N#Cc1ccc(O)c(C(=O)NC2=NCCN2)c1-c1ncccc1F]

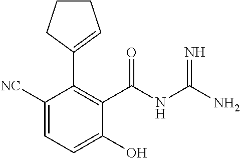 [SMILES: N#Cc1ccc(O)c(C(=O)NC(=N)N)c1C1=CCCC1]

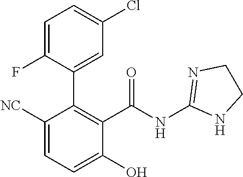 [SMILES: N#Cc1ccc(O)c(C(=O)NC2=NCCN2)c1-c1cc(Cl)ccc1F]

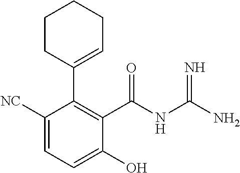 [SMILES: N#Cc1ccc(O)c(C(=O)NC(=N)N)c1C1=CCCCC1]

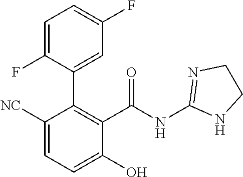 [SMILES: N#Cc1ccc(O)c(C(=O)NC2=NCCN2)c1-c1cc(F)ccc1F]

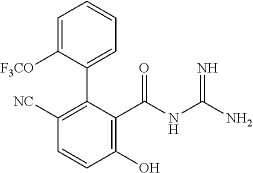 [SMILES: N#Cc1ccc(O)c(C(=O)NC(=N)N)c1-c1ccccc1OC(F)(F)F]

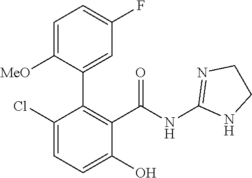 [SMILES: COc1ccc(F)cc1-c1c(Cl)ccc(O)c1C(=O)NC1=NCCN1]

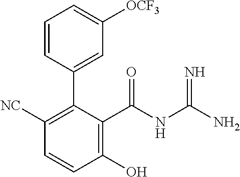 [SMILES: N#Cc1ccc(O)c(C(=O)NC(=N)N)c1-c1cccc(OC(F)(F)F)c1]

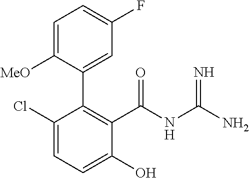 [SMILES: COc1ccc(F)cc1-c1c(Cl)ccc(O)c1C(=O)NC(=N)N]

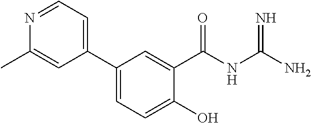 [SMILES: Cc1cc(-c2ccc(O)c(C(=O)NC(=N)N)c2)ccn1]

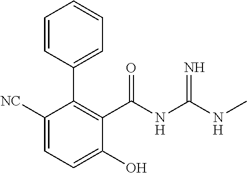 [SMILES: CNC(=N)NC(=O)c1c(O)ccc(C#N)c1-c1ccccc1]

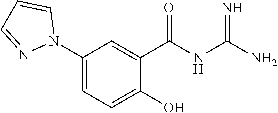 [SMILES: N=C(N)NC(=O)c1cc(-n2cccn2)ccc1O]

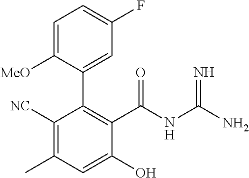 [SMILES: COc1ccc(F)cc1-c1c(C#N)c(C)cc(O)c1C(=O)NC(=N)N]

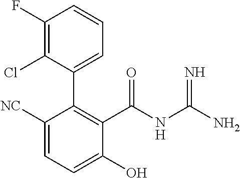 [SMILES: N#Cc1ccc(O)c(C(=O)NC(=N)N)c1-c1cccc(F)c1Cl]

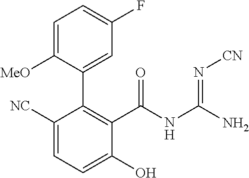 [SMILES: COc1ccc(F)cc1-c1c(C#N)ccc(O)c1C(=O)N/C(N)=N/C#N]

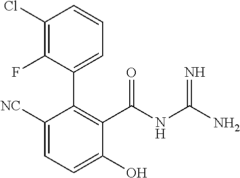 [SMILES: N#Cc1ccc(O)c(C(=O)NC(=N)N)c1-c1cccc(Cl)c1F]

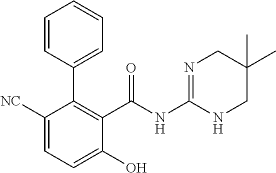 [SMILES: CC1(C)CN=C(NC(=O)c2c(O)ccc(C#N)c2-c2ccccc2)NC1]

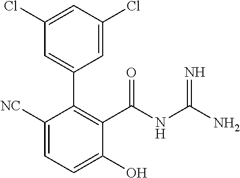 [SMILES: N#Cc1ccc(O)c(C(=O)NC(=N)N)c1-c1cc(Cl)cc(Cl)c1]

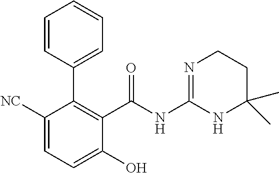 [SMILES: CC1(C)CCN=C(NC(=O)c2c(O)ccc(C#N)c2-c2ccccc2)N1]

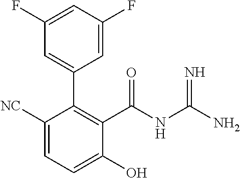 [SMILES: N#Cc1ccc(O)c(C(=O)NC(=N)N)c1-c1cc(F)cc(F)c1]

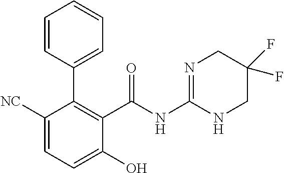 [SMILES: N#Cc1ccc(O)c(C(=O)NC2=NCC(F)(F)CN2)c1-c1ccccc1]

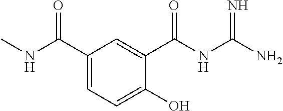 [SMILES: CNC(=O)c1ccc(O)c(C(=O)NC(=N)N)c1]

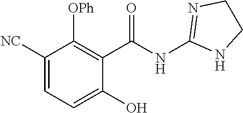 [SMILES: N#Cc1ccc(O)c(C(=O)NC2=NCCN2)c1Oc1ccccc1]

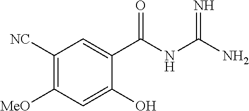 [SMILES: COc1cc(O)c(C(=O)NC(=N)N)cc1C#N]

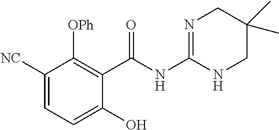 [SMILES: CC1(C)CN=C(NC(=O)c2c(O)ccc(C#N)c2Oc2ccccc2)NC1]

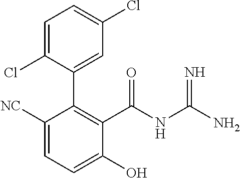 [SMILES: N#Cc1ccc(O)c(C(=O)NC(=N)N)c1-c1cc(Cl)ccc1Cl]

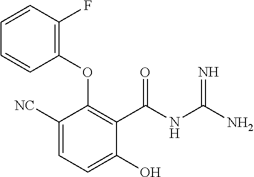 [SMILES: N#Cc1ccc(O)c(C(=O)NC(=N)N)c1Oc1ccccc1F]

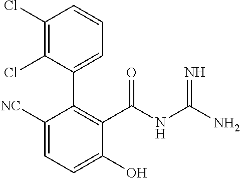 [SMILES: N#Cc1ccc(O)c(C(=O)NC(=N)N)c1-c1cccc(Cl)c1Cl]

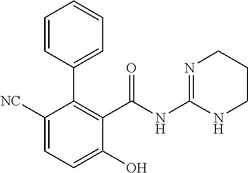 [SMILES: N#Cc1ccc(O)c(C(=O)NC2=NCCCN2)c1-c1ccccc1]

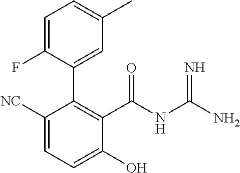 [SMILES: Cc1ccc(F)c(-c2c(C#N)ccc(O)c2C(=O)NC(=N)N)c1]

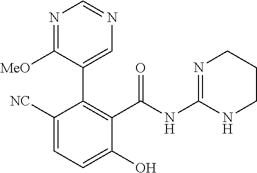 [SMILES: COc1ncncc1-c1c(C#N)ccc(O)c1C(=O)NC1=NCCCN1]

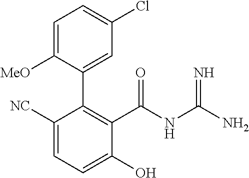 [SMILES: COc1ccc(Cl)cc1-c1c(C#N)ccc(O)c1C(=O)NC(=N)N]

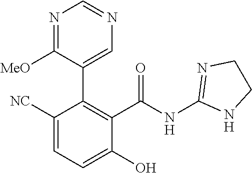 [SMILES: COc1ncncc1-c1c(C#N)ccc(O)c1C(=O)NC1=NCCN1]

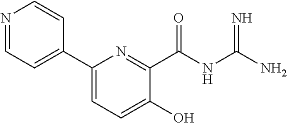 [SMILES: N=C(N)NC(=O)c1nc(-c2ccncc2)ccc1O]

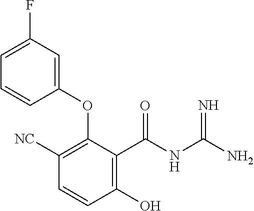 [SMILES: N#Cc1ccc(O)c(C(=O)NC(=N)N)c1Oc1cccc(F)c1]

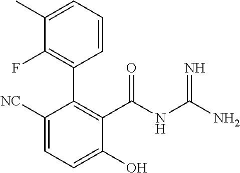 [SMILES: Cc1cccc(-c2c(C#N)ccc(O)c2C(=O)NC(=N)N)c1F]

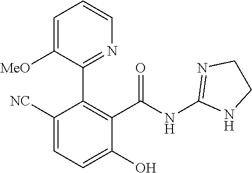 [SMILES: COc1cccnc1-c1c(C#N)ccc(O)c1C(=O)NC1=NCCN1]

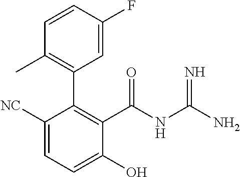 [SMILES: Cc1ccc(F)cc1-c1c(C#N)ccc(O)c1C(=O)NC(=N)N]

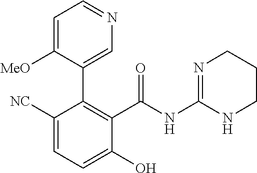 [SMILES: COc1ccncc1-c1c(C#N)ccc(O)c1C(=O)NC1=NCCCN1]

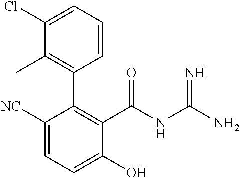 [SMILES: Cc1c(Cl)cccc1-c1c(C#N)ccc(O)c1C(=O)NC(=N)N]

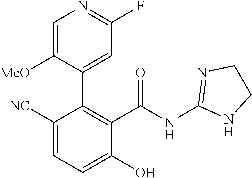 [SMILES: COc1cnc(F)cc1-c1c(C#N)ccc(O)c1C(=O)NC1=NCCN1]

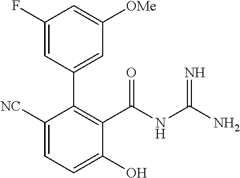 [SMILES: COc1cc(F)cc(-c2c(C#N)ccc(O)c2C(=O)NC(=N)N)c1]

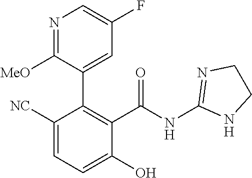 [SMILES: COc1ncc(F)cc1-c1c(C#N)ccc(O)c1C(=O)NC1=NCCN1]

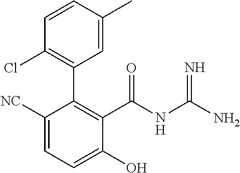 [SMILES: Cc1ccc(Cl)c(-c2c(C#N)ccc(O)c2C(=O)NC(=N)N)c1]

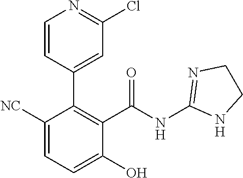 [SMILES: N#Cc1ccc(O)c(C(=O)NC2=NCCN2)c1-c1ccnc(Cl)c1]

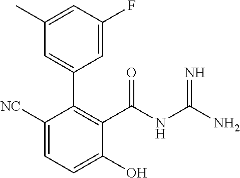 [SMILES: Cc1cc(F)cc(-c2c(C#N)ccc(O)c2C(=O)NC(=N)N)c1]

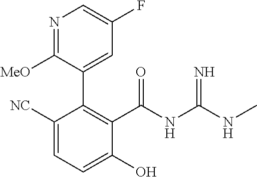 [SMILES: CNC(=N)NC(=O)c1c(O)ccc(C#N)c1-c1cc(F)cnc1OC]

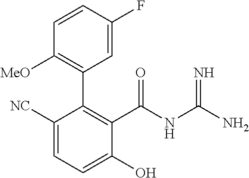 [SMILES: COc1ccc(F)cc1-c1c(C#N)ccc(O)c1C(=O)NC(=N)N]

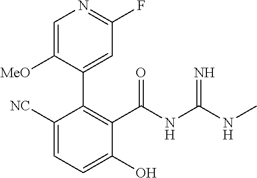 [SMILES: CNC(=N)NC(=O)c1c(O)ccc(C#N)c1-c1cc(F)ncc1OC]

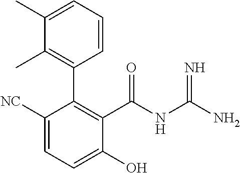 [SMILES: Cc1cccc(-c2c(C#N)ccc(O)c2C(=O)NC(=N)N)c1C]

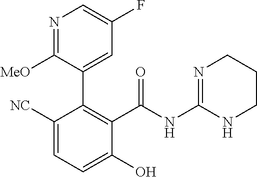 [SMILES: COc1ncc(F)cc1-c1c(C#N)ccc(O)c1C(=O)NC1=NCCCN1]

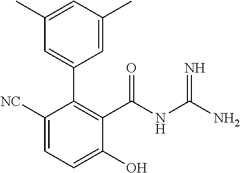 [SMILES: Cc1cc(C)cc(-c2c(C#N)ccc(O)c2C(=O)NC(=N)N)c1]

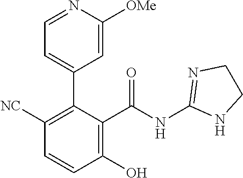 [SMILES: COc1cc(-c2c(C#N)ccc(O)c2C(=O)NC2=NCCN2)ccn1]

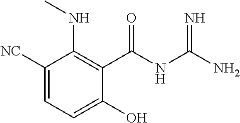 [SMILES: CNc1c(C#N)ccc(O)c1C(=O)NC(=N)N]

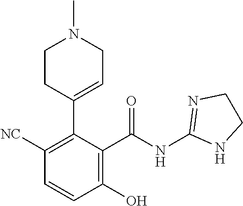 [SMILES: CN1CC=C(c2c(C#N)ccc(O)c2C(=O)NC2=NCCN2)CC1]

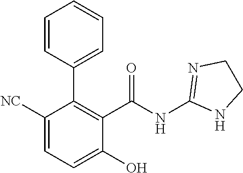 [SMILES: N#Cc1ccc(O)c(C(=O)NC2=NCCN2)c1-c1ccccc1]

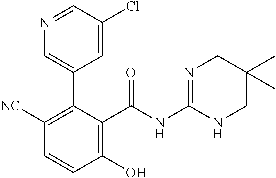 [SMILES: CC1(C)CN=C(NC(=O)c2c(O)ccc(C#N)c2-c2cncc(Cl)c2)NC1]

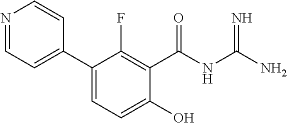 [SMILES: N=C(N)NC(=O)c1c(O)ccc(-c2ccncc2)c1F]

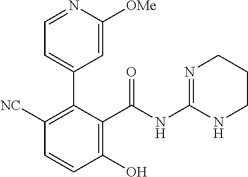 [SMILES: COc1cc(-c2c(C#N)ccc(O)c2C(=O)NC2=NCCCN2)ccn1]

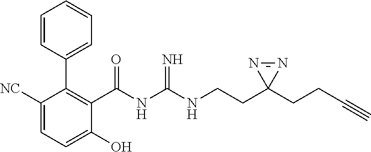 [SMILES: C#CCCC1(CCNC(=N)NC(=O)c2c(O)ccc(C#N)c2-c2ccccc2)N=N1]

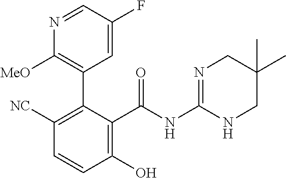 [SMILES: COc1ncc(F)cc1-c1c(C#N)ccc(O)c1C(=O)NC1=NCC(C)(C)CN1]

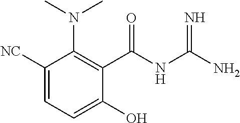 [SMILES: CN(C)c1c(C#N)ccc(O)c1C(=O)NC(=N)N]

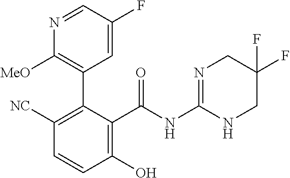 [SMILES: COc1ncc(F)cc1-c1c(C#N)ccc(O)c1C(=O)NC1=NCC(F)(F)CN1]

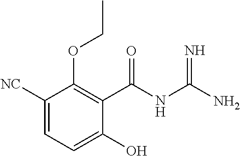 [SMILES: CCOc1c(C#N)ccc(O)c1C(=O)NC(=N)N]

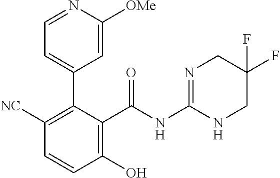 [SMILES: COc1cc(-c2c(C#N)ccc(O)c2C(=O)NC2=NCC(F)(F)CN2)ccn1]

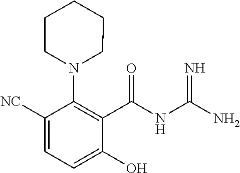 [SMILES: N#Cc1ccc(O)c(C(=O)NC(=N)N)c1N1CCCCC1]

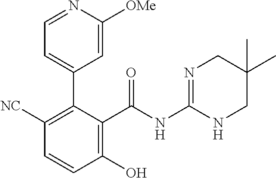 [SMILES: COc1cc(-c2c(C#N)ccc(O)c2C(=O)NC2=NCC(C)(C)CN2)ccn1]

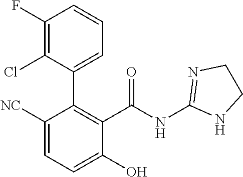 [SMILES: N#Cc1ccc(O)c(C(=O)NC2=NCCN2)c1-c1cccc(F)c1Cl]

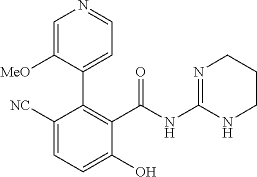 [SMILES: COc1cnccc1-c1c(C#N)ccc(O)c1C(=O)NC1=NCCCN1]

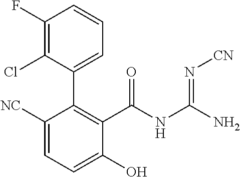 [SMILES: N#C/N=C(\N)NC(=O)c1c(O)ccc(C#N)c1-c1cccc(F)c1Cl]

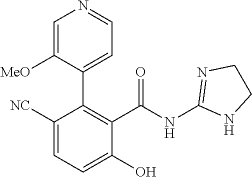 [SMILES: COc1cnccc1-c1c(C#N)ccc(O)c1C(=O)NC1=NCCN1]

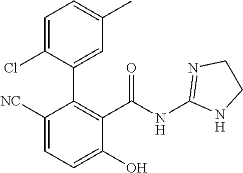 [SMILES: Cc1ccc(Cl)c(-c2c(C#N)ccc(O)c2C(=O)NC2=NCCN2)c1]

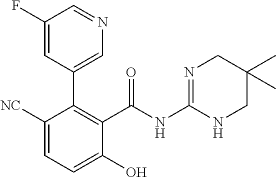 [SMILES: CC1(C)CN=C(NC(=O)c2c(O)ccc(C#N)c2-c2cncc(F)c2)NC1]

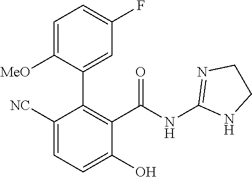 [SMILES: COc1ccc(F)cc1-c1c(C#N)ccc(O)c1C(=O)NC1=NCCN1]

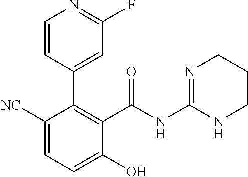 [SMILES: N#Cc1ccc(O)c(C(=O)NC2=NCCCN2)c1-c1ccnc(F)c1]

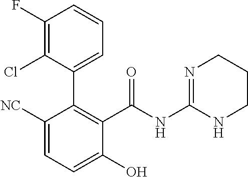 [SMILES: N#Cc1ccc(O)c(C(=O)NC2=NCCCN2)c1-c1cccc(F)c1Cl]

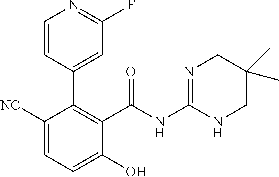 [SMILES: CC1(C)CN=C(NC(=O)c2c(O)ccc(C#N)c2-c2ccnc(F)c2)NC1]

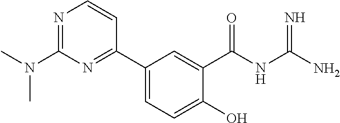 [SMILES: CN(C)c1nccc(-c2ccc(O)c(C(=O)NC(=N)N)c2)n1]

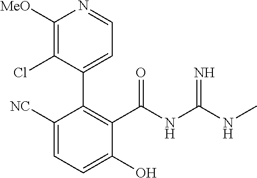 [SMILES: CNC(=N)NC(=O)c1c(O)ccc(C#N)c1-c1ccnc(OC)c1Cl]

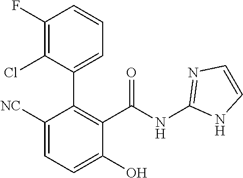 [SMILES: N#Cc1ccc(O)c(C(=O)Nc2ncc[nH]2)c1-c1cccc(F)c1Cl]

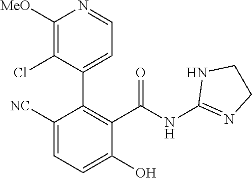 [SMILES: COc1nccc(-c2c(C#N)ccc(O)c2C(=O)NC2=NCCN2)c1Cl]

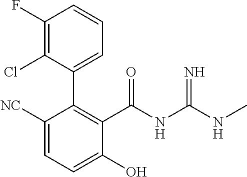 [SMILES: CNC(=N)NC(=O)c1c(O)ccc(C#N)c1-c1cccc(F)c1Cl]

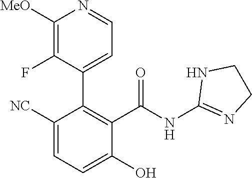 [SMILES: COc1nccc(-c2c(C#N)ccc(O)c2C(=O)NC2=NCCN2)c1F]

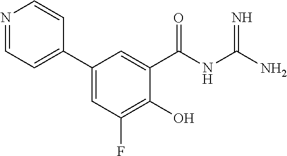 [SMILES: N=C(N)NC(=O)c1cc(-c2ccncc2)cc(F)c1O]

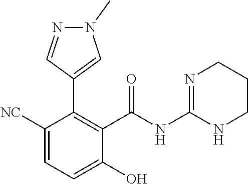 [SMILES: Cn1cc(-c2c(C#N)ccc(O)c2C(=O)NC2=NCCCN2)cn1]

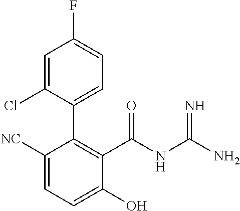 [SMILES: N#Cc1ccc(O)c(C(=O)NC(=N)N)c1-c1ccc(F)cc1Cl]

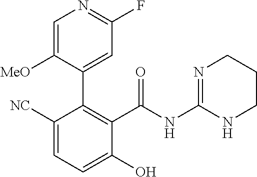 [SMILES: COc1cnc(F)cc1-c1c(C#N)ccc(O)c1C(=O)NC1=NCCCN1]

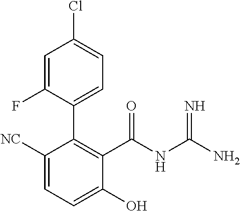 [SMILES: N#Cc1ccc(O)c(C(=O)NC(=N)N)c1-c1ccc(Cl)cc1F]

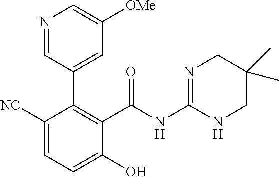 [SMILES: COc1cncc(-c2c(C#N)ccc(O)c2C(=O)NC2=NCC(C)(C)CN2)c1]

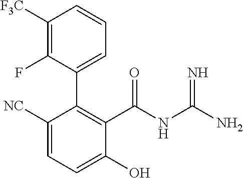 [SMILES: N#Cc1ccc(O)c(C(=O)NC(=N)N)c1-c1cccc(C(F)(F)F)c1F]

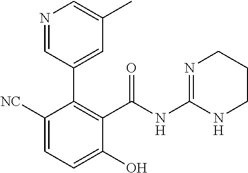 [SMILES: Cc1cncc(-c2c(C#N)ccc(O)c2C(=O)NC2=NCCCN2)c1]

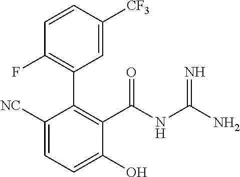 [SMILES: N#Cc1ccc(O)c(C(=O)NC(=N)N)c1-c1cc(C(F)(F)F)ccc1F]

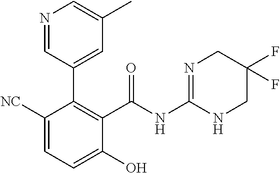 [SMILES: Cc1cncc(-c2c(C#N)ccc(O)c2C(=O)NC2=NCC(F)(F)CN2)c1]

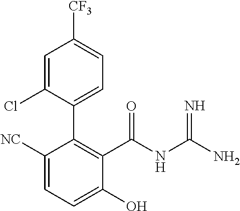 [SMILES: N#Cc1ccc(O)c(C(=O)NC(=N)N)c1-c1ccc(C(F)(F)F)cc1Cl]

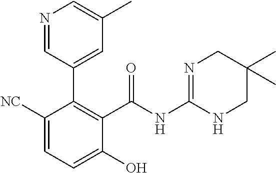 [SMILES: Cc1cncc(-c2c(C#N)ccc(O)c2C(=O)NC2=NCC(C)(C)CN2)c1]

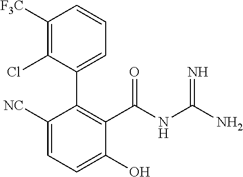 [SMILES: N#Cc1ccc(O)c(C(=O)NC(=N)N)c1-c1cccc(C(F)(F)F)c1Cl]

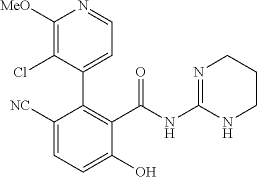 [SMILES: COc1nccc(-c2c(C#N)ccc(O)c2C(=O)NC2=NCCCN2)c1Cl]

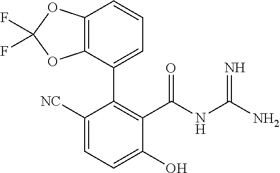 [SMILES: N#Cc1ccc(O)c(C(=O)NC(=N)N)c1-c1cccc2c1OC(F)(F)O2]

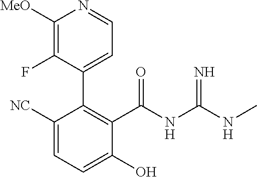 [SMILES: CNC(=N)NC(=O)c1c(O)ccc(C#N)c1-c1ccnc(OC)c1F]

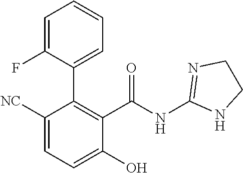 [SMILES: N#Cc1ccc(O)c(C(=O)NC2=NCCN2)c1-c1ccccc1F]

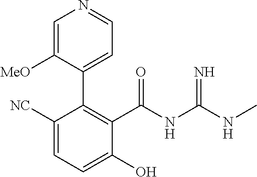 [SMILES: CNC(=N)NC(=O)c1c(O)ccc(C#N)c1-c1ccncc1OC]

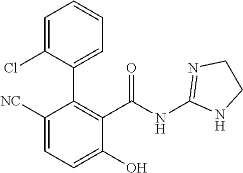 [SMILES: N#Cc1ccc(O)c(C(=O)NC2=NCCN2)c1-c1ccccc1Cl]

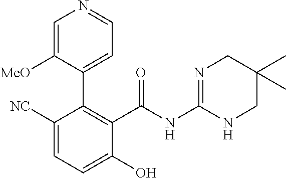 [SMILES: COc1cnccc1-c1c(C#N)ccc(O)c1C(=O)NC1=NCC(C)(C)CN1]

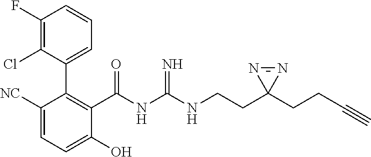 [SMILES: C#CCCC1(CCNC(=N)NC(=O)c2c(O)ccc(C#N)c2-c2cccc(F)c2Cl)N=N1]

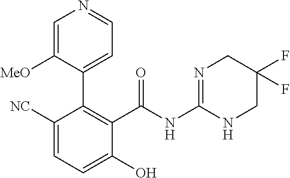 [SMILES: COc1cnccc1-c1c(C#N)ccc(O)c1C(=O)NC1=NCC(F)(F)CN1]

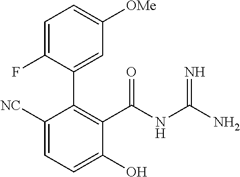 [SMILES: COc1ccc(F)c(-c2c(C#N)ccc(O)c2C(=O)NC(=N)N)c1]

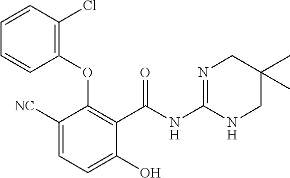 [SMILES: CC1(C)CN=C(NC(=O)c2c(O)ccc(C#N)c2Oc2ccccc2Cl)NC1]

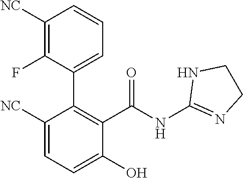 [SMILES: N#Cc1cccc(-c2c(C#N)ccc(O)c2C(=O)NC2=NCCN2)c1F]

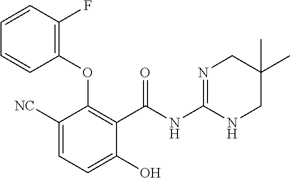 [SMILES: CC1(C)CN=C(NC(=O)c2c(O)ccc(C#N)c2Oc2ccccc2F)NC1]

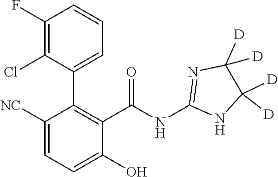 [SMILES: [2H]C1([2H])N=C(NC(=O)c2c(O)ccc(C#N)c2-c2cccc(F)c2Cl)NC1([2H])[2H]]

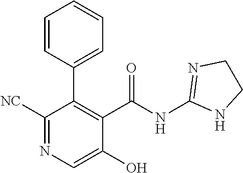 [SMILES: N#Cc1ncc(O)c(C(=O)NC2=NCCN2)c1-c1ccccc1]

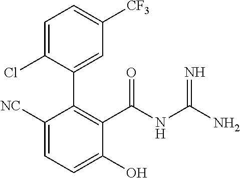 [SMILES: N#Cc1ccc(O)c(C(=O)NC(=N)N)c1-c1cc(C(F)(F)F)ccc1Cl]

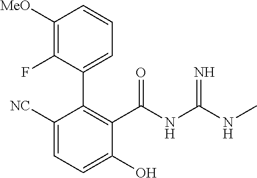 [SMILES: CNC(=N)NC(=O)c1c(O)ccc(C#N)c1-c1cccc(OC)c1F]

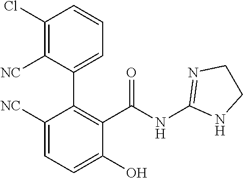 [SMILES: N#Cc1ccc(O)c(C(=O)NC2=NCCN2)c1-c1cccc(Cl)c1C#N]

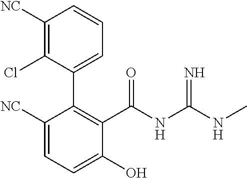 [SMILES: CNC(=N)NC(=O)c1c(O)ccc(C#N)c1-c1cccc(C#N)c1Cl]

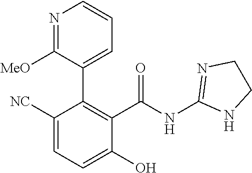 [SMILES: COc1ncccc1-c1c(C#N)ccc(O)c1C(=O)NC1=NCCN1]

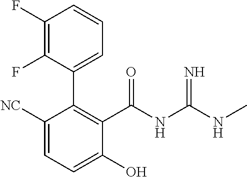 [SMILES: CNC(=N)NC(=O)c1c(O)ccc(C#N)c1-c1cccc(F)c1F]

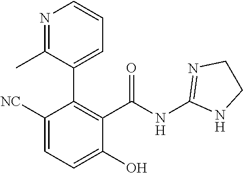 [SMILES: Cc1ncccc1-c1c(C#N)ccc(O)c1C(=O)NC1=NCCN1]

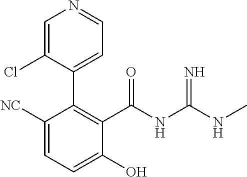 [SMILES: CNC(=N)NC(=O)c1c(O)ccc(C#N)c1-c1ccncc1Cl]

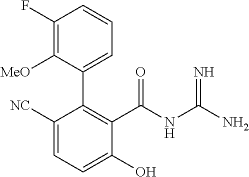 [SMILES: COc1c(F)cccc1-c1c(C#N)ccc(O)c1C(=O)NC(=N)N]

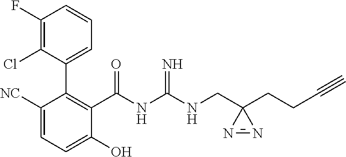 [SMILES: C#CCCC1(CNC(=N)NC(=O)c2c(O)ccc(C#N)c2-c2cccc(F)c2Cl)N=N1]

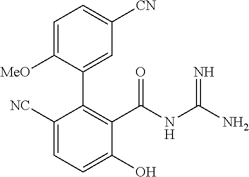 [SMILES: COc1ccc(C#N)cc1-c1c(C#N)ccc(O)c1C(=O)NC(=N)N]

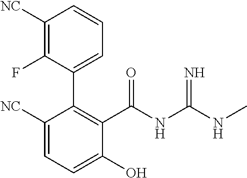 [SMILES: CNC(=N)NC(=O)c1c(O)ccc(C#N)c1-c1cccc(C#N)c1F]

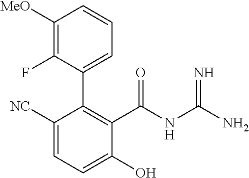 [SMILES: COc1cccc(-c2c(C#N)ccc(O)c2C(=O)NC(=N)N)c1F]

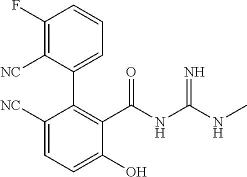 [SMILES: CNC(=N)NC(=O)c1c(O)ccc(C#N)c1-c1cccc(F)c1C#N]

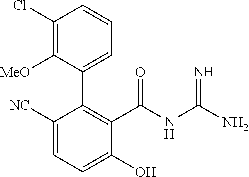 [SMILES: COc1c(Cl)cccc1-c1c(C#N)ccc(O)c1C(=O)NC(=N)N]

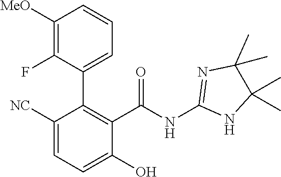 [SMILES: COc1cccc(-c2c(C#N)ccc(O)c2C(=O)NC2=NC(C)(C)C(C)(C)N2)c1F]

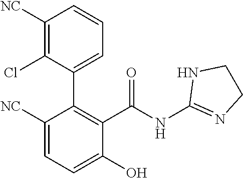 [SMILES: N#Cc1cccc(-c2c(C#N)ccc(O)c2C(=O)NC2=NCCN2)c1Cl]

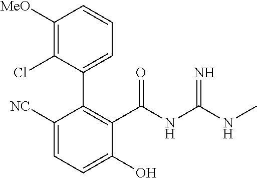 [SMILES: CNC(=N)NC(=O)c1c(O)ccc(C#N)c1-c1cccc(OC)c1Cl]

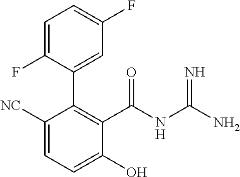 [SMILES: N#Cc1ccc(O)c(C(=O)NC(=N)N)c1-c1cc(F)ccc1F]

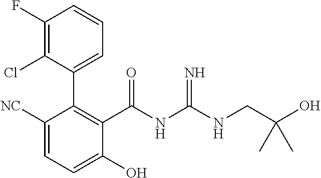 [SMILES: CC(C)(O)CNC(=N)NC(=O)c1c(O)ccc(C#N)c1-c1cccc(F)c1Cl]

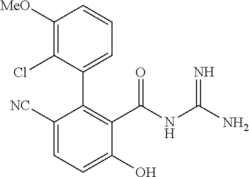 [SMILES: COc1cccc(-c2c(C#N)ccc(O)c2C(=O)NC(=N)N)c1Cl]

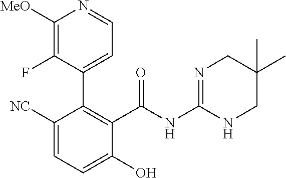 [SMILES: COc1nccc(-c2c(C#N)ccc(O)c2C(=O)NC2=NCC(C)(C)CN2)c1F]

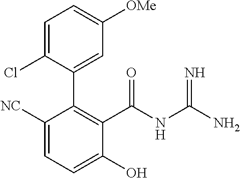 [SMILES: COc1ccc(Cl)c(-c2c(C#N)ccc(O)c2C(=O)NC(=N)N)c1]

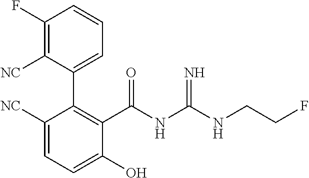 [SMILES: N#Cc1ccc(O)c(C(=O)NC(=N)NCCF)c1-c1cccc(F)c1C#N]

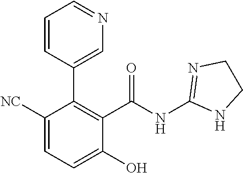 [SMILES: N#Cc1ccc(O)c(C(=O)NC2=NCCN2)c1-c1cccnc1]

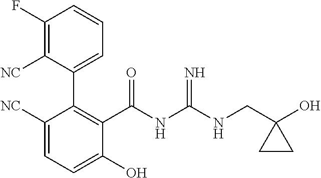 [SMILES: N#Cc1ccc(O)c(C(=O)NC(=N)NCC2(O)CC2)c1-c1cccc(F)c1C#N]

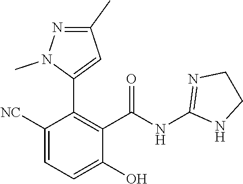 [SMILES: Cc1cc(-c2c(C#N)ccc(O)c2C(=O)NC2=NCCN2)n(C)n1]

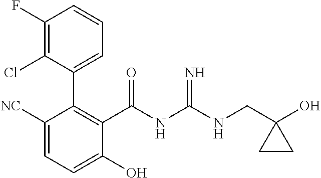 [SMILES: N#Cc1ccc(O)c(C(=O)NC(=N)NCC2(O)CC2)c1-c1cccc(F)c1Cl]

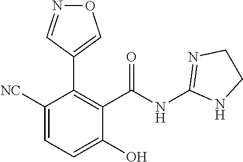 [SMILES: N#Cc1ccc(O)c(C(=O)NC2=NCCN2)c1-c1cnoc1]

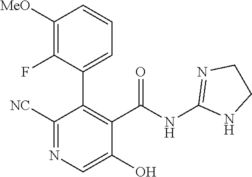 [SMILES: COc1cccc(-c2c(C#N)ncc(O)c2C(=O)NC2=NCCN2)c1F]

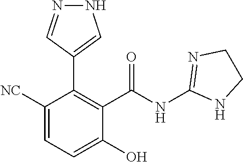 [SMILES: N#Cc1ccc(O)c(C(=O)NC2=NCCN2)c1-c1cn[nH]c1]

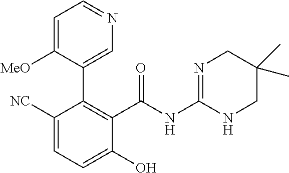 [SMILES: COc1ccncc1-c1c(C#N)ccc(O)c1C(=O)NC1=NCC(C)(C)CN1]

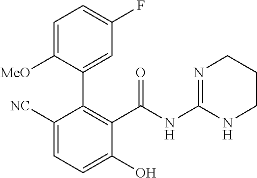 [SMILES: COc1ccc(F)cc1-c1c(C#N)ccc(O)c1C(=O)NC1=NCCCN1]

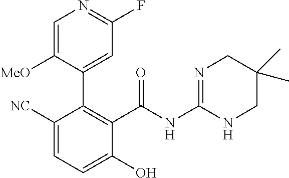 [SMILES: COc1cnc(F)cc1-c1c(C#N)ccc(O)c1C(=O)NC1=NCC(C)(C)CN1]

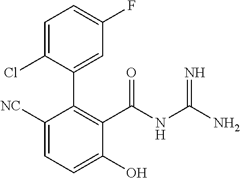 [SMILES: N#Cc1ccc(O)c(C(=O)NC(=N)N)c1-c1cc(F)ccc1Cl]

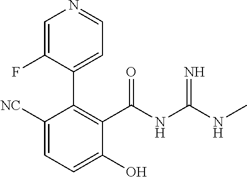 [SMILES: CNC(=N)NC(=O)c1c(O)ccc(C#N)c1-c1ccncc1F]

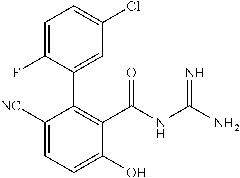 [SMILES: N#Cc1ccc(O)c(C(=O)NC(=N)N)c1-c1cc(Cl)ccc1F]

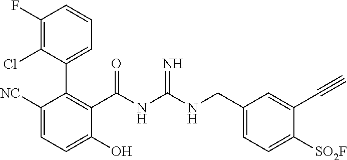 [SMILES: C#Cc1cc(CNC(=N)NC(=O)c2c(O)ccc(C#N)c2-c2cccc(F)c2Cl)ccc1S(=O)(=O)F]

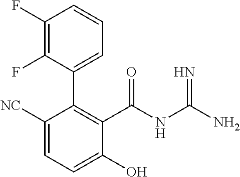 [SMILES: N#Cc1ccc(O)c(C(=O)NC(=N)N)c1-c1cccc(F)c1F]

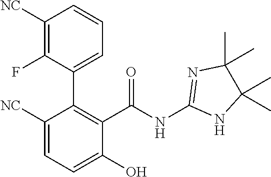 [SMILES: CC1(C)N=C(NC(=O)c2c(O)ccc(C#N)c2-c2cccc(C#N)c2F)NC1(C)C]

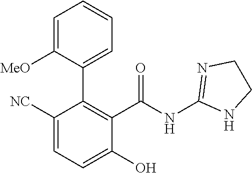 [SMILES: COc1ccccc1-c1c(C#N)ccc(O)c1C(=O)NC1=NCCN1]

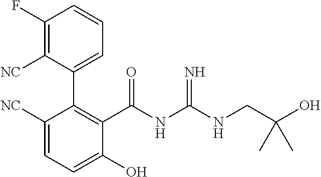 [SMILES: CC(C)(O)CNC(=N)NC(=O)c1c(O)ccc(C#N)c1-c1cccc(F)c1C#N]

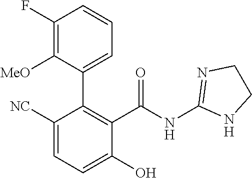 [SMILES: COc1c(F)cccc1-c1c(C#N)ccc(O)c1C(=O)NC1=NCCN1]

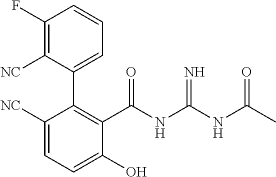 [SMILES: CC(=O)NC(=N)NC(=O)c1c(O)ccc(C#N)c1-c1cccc(F)c1C#N]

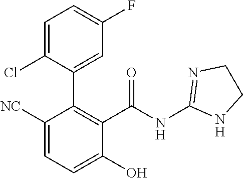 [SMILES: N#Cc1ccc(O)c(C(=O)NC2=NCCN2)c1-c1cc(F)ccc1Cl]

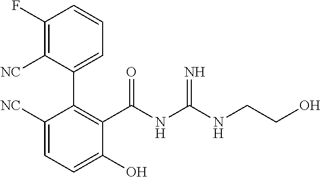 [SMILES: N#Cc1ccc(O)c(C(=O)NC(=N)NCCO)c1-c1cccc(F)c1C#N]

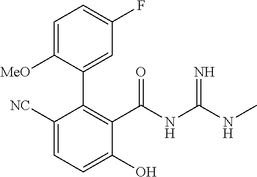 [SMILES: CNC(=N)NC(=O)c1c(O)ccc(C#N)c1-c1cc(F)ccc1OC]

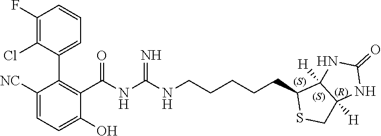

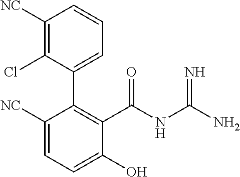 [SMILES: N#Cc1cccc(-c2c(C#N)ccc(O)c2C(=O)NC(=N)N)c1Cl]

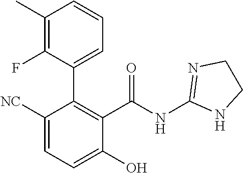 [SMILES: Cc1cccc(-c2c(C#N)ccc(O)c2C(=O)NC2=NCCN2)c1F]

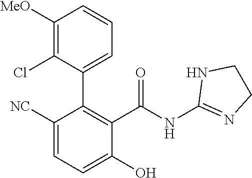 [SMILES: COc1cccc(-c2c(C#N)ccc(O)c2C(=O)NC2=NCCN2)c1Cl]

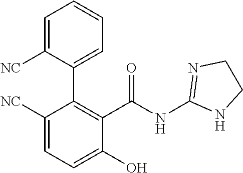 [SMILES: N#Cc1ccccc1-c1c(C#N)ccc(O)c1C(=O)NC1=NCCN1]

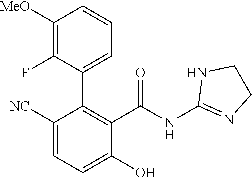 [SMILES: COc1cccc(-c2c(C#N)ccc(O)c2C(=O)NC2=NCCN2)c1F]

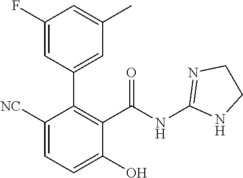 [SMILES: Cc1cc(F)cc(-c2c(C#N)ccc(O)c2C(=O)NC2=NCCN2)c1]

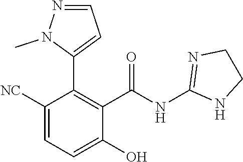 [SMILES: Cn1nccc1-c1c(C#N)ccc(O)c1C(=O)NC1=NCCN1]

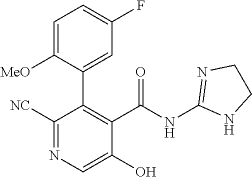 [SMILES: COc1ccc(F)cc1-c1c(C#N)ncc(O)c1C(=O)NC1=NCCN1]

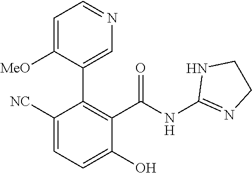 [SMILES: COc1ccncc1-c1c(C#N)ccc(O)c1C(=O)NC1=NCCN1]

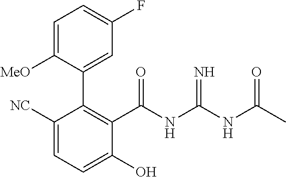 [SMILES: COc1ccc(F)cc1-c1c(C#N)ccc(O)c1C(=O)NC(=N)NC(C)=O]

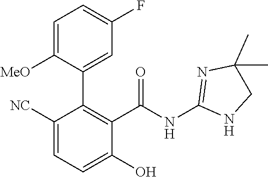 [SMILES: COc1ccc(F)cc1-c1c(C#N)ccc(O)c1C(=O)NC1=NC(C)(C)CN1]

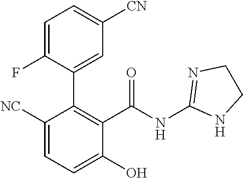 [SMILES: N#Cc1ccc(F)c(-c2c(C#N)ccc(O)c2C(=O)NC2=NCCN2)c1]

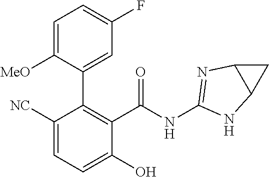 [SMILES: COc1ccc(F)cc1-c1c(C#N)ccc(O)c1C(=O)NC1=NC2CC2N1]

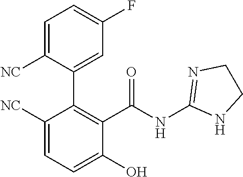 [SMILES: N#Cc1ccc(F)cc1-c1c(C#N)ccc(O)c1C(=O)NC1=NCCN1]

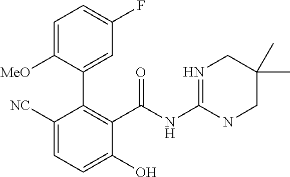 [SMILES: COc1ccc(F)cc1-c1c(C#N)ccc(O)c1C(=O)NC1=NCC(C)(C)CN1]

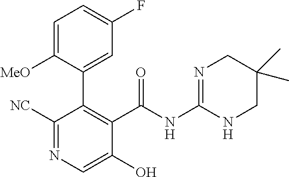 [SMILES: COc1ccc(F)cc1-c1c(C#N)ncc(O)c1C(=O)NC1=NCC(C)(C)CN1]

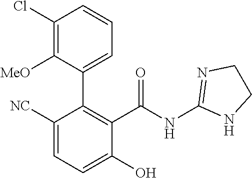 [SMILES: COc1c(Cl)cccc1-c1c(C#N)ccc(O)c1C(=O)NC1=NCCN1]

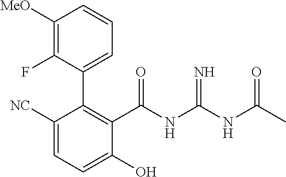 [SMILES: COc1cccc(-c2c(C#N)ccc(O)c2C(=O)NC(=N)NC(C)=O)c1F]

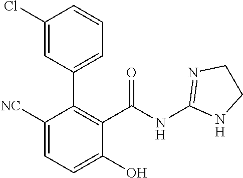 [SMILES: N#Cc1ccc(O)c(C(=O)NC2=NCCN2)c1-c1cccc(Cl)c1]

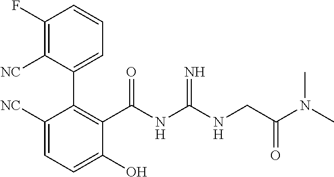 [SMILES: CN(C)C(=O)CNC(=N)NC(=O)c1c(O)ccc(C#N)c1-c1cccc(F)c1C#N]

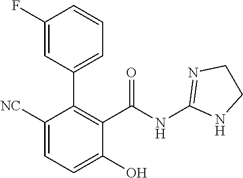 [SMILES: N#Cc1ccc(O)c(C(=O)NC2=NCCN2)c1-c1cccc(F)c1]

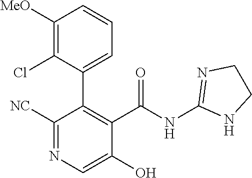 [SMILES: COc1cccc(-c2c(C#N)ncc(O)c2C(=O)NC2=NCCN2)c1Cl]

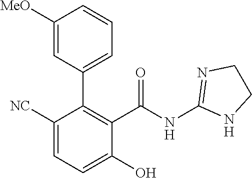 [SMILES: COc1cccc(-c2c(C#N)ccc(O)c2C(=O)NC2=NCCN2)c1]

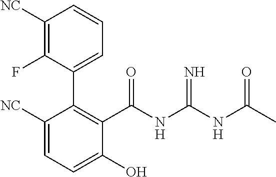 [SMILES: CC(=O)NC(=N)NC(=O)c1c(O)ccc(C#N)c1-c1cccc(C#N)c1F]

In some embodiments, the compounds are atropisomers.

Additionally, unless otherwise stated, structures depicted herein are also meant to include compounds that differ only in the presence of one or more isotopically enriched atoms. For example, compounds produced by the replacement of a hydrogen with deuterium or tritium, or of a carbon with a ¹³C- or ¹⁴C-enriched carbon are within the scope of this invention. Such compounds are useful, for example, as analytical tools, as probes in biological assays, or as therapeutic agents in accordance with the present invention. For example, in the case of variable R¹, the (C₁-C₄)alkyl or the —O—(C₁-C₄)alkyl can be suitably deuterated (e.g., —CD₃, —OCD₃).

Any compound of the invention can also be radiolabel for the preparation of a radiopharmaceutical agent.

### Methods of Treatment

Creatine Transporter Deficiency (CTD)

CTD is an inborn error of creatine metabolism in which creatine is not properly transported to the brain and muscles due to defective creatine transporters. CTD is an X-linked disorder caused by mutations in the SLC6A8 gene. The SLC6A8 gene is located on the short arm of the sex chromosome, Xq28. Hemizygous males with CTD express speech and behavior abnormalities, intellectual disabilities, development delay, seizures, and autistic behavior. Heterozygous females with CTD generally express fewer, less severe symptoms. CTD is one of three different types of cerebral creatine deficiency (CCD). The other two types of CCD are guanidinoacetate methyltransferase (GAMT) deficiency and L-arginine:glycine amidinotransferase (AGAT) deficiency. Clinical presentation of CTD is similar to that of GA IT and AGAT deficiency. CTD was first identified in 2001 with the presence of a hemizygous nonsense mutation in the SLC6A8 gene in a male patient.

CTD is difficult to treat because the actual transporter responsible for transporting creatine to the brain and muscles is defective. Studies in which oral creatine monohydrate supplements were given to patients with CTD found that patients did not respond to treatment. However, similar studies conducted in which patients that had GAMT or AGAT deficiency were given oral creatine monohydrate supplements found that patient's clinical symptoms improved. Patients with CTD are unresponsive to oral creatine monohydrate supplements because regardless of the amount of creatine they ingest, the creatine transporter is still defective, and therefore creatine is incapable of being transported across the BBB.

Accordingly, in certain embodiments, the invention provides methods of disease or disorder associated with a SLC6A8 mutation, comprising administering to a mammal in need thereof a therapeutically effective amount of a compound or a pharmaceutical composition of the invention.

In some embodiments, the disease or disorder is creatine transporter deficiency. In some embodiments, the disease or disorder is motor dysfunction. In some embodiments, the disease or disorder is intellectual disability. In some embodiments, the disease or disorder is language delay or speech delay. In some embodiments, the disease or disorder is hypotonia. In some embodiments, the disease or disorder is seizures. In some embodiments, the disease or disorder is behaviors associated with autism and attention deficit hyperactivity disorder. In some embodiments, the disease or disorder is fatigue. In some embodiments, the disease or disorder is muscular hypotonia. In some embodiments, the disease or disorder is low weight gain. In some embodiments, the disease or disorder is gastrointestinal disorders. In some embodiments, the disease or disorder is cardiac disorders.

In certain embodiments, the invention provides methods of increasing cellular trafficking to the membrane of a creatine transporter, comprising administering to a subject in need thereof an effective amount of a compound of the invention.

In some embodiments, the creatine transporter is SLC6A8. In some embodiments, the creatine transporter is a mutant creatine transporter.

In certain embodiments, the invention provides methods of correcting a defect in cellular creatine transporter function, comprising administering to a subject in need thereof an effective amount of a compound of the invention.

In some embodiments, the creatine transporter is SLC6A8. In some embodiments, the creatine transporter is a mutant creatine transporter. In some embodiments, the cellular concentration of creatine is increased.

In some embodiments, the invention relates to method of decreasing accumulation or the concentration of guanidinoacetic acid or a salt thereof in a cell, comprising administering to a mammal in need thereof a therapeutically effective amount of a compound that increases transport of guanidinoacetic acid or a salt thereof by a mutant creatine transporter.

In some embodiments, the creatine transporter is SLC6A8. In some embodiments, the creatine transporter is a mutant creatine transporter.

In some embodiments, the compound decreases intracellular accumulation of guanidinoacetic acid or a salt thereof. In some embodiments, the compound decreases the intracellular concentration of guanidinoacetic acid or a salt thereof.

In some embodiments, the invention relates to methods of increasing transport of guanidinoacetic acid or a salt thereof across the blood-brain barrier, comprising administering to a mammal in need thereof a therapeutically effective amount of a compound that increases transport of guanidinoacetic acid or a salt thereof by a mutant creatine transporter. In some embodiments, the mutant creatine transporter is mutant SLC6A8.

Pharmaceutical Compositions, Routes of Administration, and Dosing

In certain embodiments, the invention is directed to a pharmaceutical composition, comprising a compound of the invention and a pharmaceutically acceptable carrier. In certain embodiments, the pharmaceutical composition comprises a plurality of compounds of the invention and a pharmaceutically acceptable carrier.

In certain embodiments, a pharmaceutical composition of the invention further comprises at least one additional pharmaceutically active agent other than a compound of the invention. The at least one additional pharmaceutically active agent can be an agent useful in the treatment of ischemia-reperfusion injury.

Pharmaceutical compositions of the invention can be prepared by combining one or more compounds of the invention with a pharmaceutically acceptable carrier and, optionally, one or more additional pharmaceutically active agents.

As stated above, an “effective amount” refers to any amount that is sufficient to achieve a desired biological effect. Combined with the teachings provided herein, by choosing among the various active compounds and weighing factors such as potency, relative bioavailability, patient body weight, severity of adverse side-effects and mode of administration, an effective prophylactic or therapeutic treatment regimen can be planned which does not cause substantial unwanted toxicity and yet is effective to treat the particular subject. The effective amount for any particular application can vary depending on such factors as the disease or condition being treated, the particular compound of the invention being administered, the size of the subject, or the severity of the disease or condition. One of ordinary skill in the art can empirically determine the effective amount of a particular compound of the invention and/or other therapeutic agent without necessitating undue experimentation. A maximum dose may be used, that is, the highest safe dose according to some medical judgment. Multiple doses per day may be contemplated to achieve appropriate systemic levels of compounds. Appropriate systemic levels can be determined by, for example, measurement of the patient's peak or sustained plasma level of the drug. “Dose” and “dosage” are used interchangeably herein.

In certain embodiments, intravenous administration of a compound may typically be from 0.1 mg/kg/day to 20 mg/kg/day. In one embodiment, intravenous administration of a compound may typically be from 0.1 mg/kg/day to 2 mg/kg/day. In one embodiment, intravenous administration of a compound may typically be from 0.5 mg/kg/day to 5 mg/kg/day. In one embodiment, intravenous administration of a compound may typically be from 1 mg/kg/day to 20 mg/kg/day. In one embodiment, intravenous administration of a compound may typically be from 1 mg/kg/day to 10 mg/kg/day.

Generally, daily oral doses of a compound will be, for human subjects, from about 0.01 milligrams/kg per day to 1000 milligrams/kg per day. It is expected that oral doses in the range of 0.5 to 50 milligrams/kg, in one or more administrations per day, will yield therapeutic results.

Dosage may be adjusted appropriately to achieve desired drug levels, local or systemic, depending upon the mode of administration. For example, it is expected that intravenous administration would be from one order to several orders of magnitude lower dose per day. In the event that the response in a subject is insufficient at such doses, even higher doses (or effective higher doses by a different, more localized delivery route) may be employed to the extent that patient tolerance permits. Multiple doses per day are contemplated to achieve appropriate systemic levels of the compound.

For any compound described herein the therapeutically effective amount can be initially determined from animal models. A therapeutically effective dose can also be determined from human data for compounds which have been tested in humans and for compounds which are known to exhibit similar pharmacological activities, such as other related active agents. Higher doses may be required for parenteral administration. The applied dose can be adjusted based on the relative bioavailability and potency of the administered compound. Adjusting the dose to achieve maximal efficacy based on the methods described above and other methods as are well-known in the art is well within the capabilities of the ordinarily skilled artisan.

The formulations of the invention can be administered in pharmaceutically acceptable solutions, which may routinely contain pharmaceutically acceptable concentrations of salt, buffering agents, preservatives, compatible carriers, adjuvants, and optionally other therapeutic ingredients.

For use in therapy, an effective amount of the compound can be administered to a subject by any mode that delivers the compound to the desired surface. Administering a pharmaceutical composition may be accomplished by any means known to the skilled artisan. Routes of administration include but are not limited to intravenous, intramuscular, intraperitoneal, intravesical (urinary bladder), oral, subcutaneous, direct injection (for example, into a tumor or abscess), mucosal (e.g., topical to eye), inhalation, and topical.

For intravenous and other parenteral routes of administration, a compound of the invention can be formulated as a lyophilized preparation, as a lyophilized preparation of liposome-intercalated or -encapsulated active compound, as a lipid complex in aqueous suspension, or as a salt complex. Lyophilized formulations are generally reconstituted in suitable aqueous solution, e.g., in sterile water or saline, shortly prior to administration.

For oral administration, the compounds can be formulated readily by combining the active compound(s) with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers, e.g., EDTA for neutralizing internal acid conditions or may be administered without any carriers.

Also specifically contemplated are oral dosage forms of the above component or components. The component or components may be chemically modified so that oral delivery of the derivative is efficacious. Generally, the chemical modification contemplated is the attachment of at least one moiety to the component molecule itself, where said moiety permits (a) inhibition of acid hydrolysis; and (b) uptake into the blood stream from the stomach or intestine. Also desired is the increase in overall stability of the component or components and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline. Abuchowski and Davis, “Soluble Polymer-Enzyme Adducts”, In: Enzymes as Drugs, Hocenberg and Roberts, eds., Wiley-Interscience, New York, N.Y., pp. 367-383 (1981); Newmark et al., J Appl Biochem 4:185-9 (1982). Other polymers that could be used are poly-1,3-dioxolane and poly-1,3,6-tioxocane. For pharmaceutical usage, as indicated above, polyethylene glycol moieties are suitable.

For the component (or derivative) the location of release may be the stomach, the small intestine (the duodenum, the jejunum, or the ileum), or the large intestine. One skilled in the art has available formulations which will not dissolve in the stomach, yet will release the material in the duodenum or elsewhere in the intestine. Preferably, the release will avoid the deleterious effects of the stomach environment, either by protection of the compound of the invention (or derivative) or by release of the biologically active material beyond the stomach environment, such as in the intestine.

To ensure full gastric resistance a coating impermeable to at least pH 5.0 is essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and shellac. These coatings may be used as mixed films.

A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic (e.g., powder); for liquid forms, a soft gelatin shell may be used. The shell material of cachets could be thick starch or other edible paper. For pills, lozenges, molded tablets or tablet triturates, moist massing techniques can be used.

The therapeutic can be included in the formulation as fine multi-particulates in the form of granules or pellets of particle size about 1 mm. The formulation of the material for capsule administration could also be as a powder, lightly compressed plugs or even as tablets. The therapeutic could be prepared by compression.

Colorants and flavoring agents may all be included. For example, the compound of the invention (or derivative) may be formulated (such as by liposome or microsphere encapsulation) and then further contained within an edible product, such as a refrigerated beverage containing colorants and flavoring agents.

One may dilute or increase the volume of the therapeutic with an inert material. These diluents could include carbohydrates, especially mannitol, α-lactose, anhydrous lactose, cellulose, sucrose, modified dextrans and starch. Certain inorganic salts may be also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the therapeutic into a solid dosage form. Materials used as disintegrates include but are not limited to starch, including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, Karaya or tragacanth. Alginic acid and its sodium salt are also useful as disintegrants.

Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

An anti-frictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000.

Glidants that might improve the flow properties of the drug during formulation and to aid rearrangement during compression might be added. The glidants may include starch, talc, pyrogenic silica and hydrated silicoaluminate.

To aid dissolution of the therapeutic into the aqueous environment a surfactant might be added as a wetting agent. Surfactants may include anionic detergents such as sodium lauryl sulfate, dioctyl sodium sulfosuccinate and dioctyl sodium sulfonate. Cationic detergents which can be used and can include benzalkonium chloride and benzethonium chloride. Potential non-ionic detergents that could be included in the formulation as surfactants include lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the compound of the invention or derivative either alone or as a mixture in different ratios.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For topical administration, the compound may be formulated as solutions, gels, ointments, creams, suspensions, etc. as are well-known in the art. Systemic formulations include those designed for administration by injection, e.g., subcutaneous, intravenous, intramuscular, intrathecal or intraperitoneal injection, as well as those designed for transdermal, transmucosal oral or pulmonary administration.

For administration by inhalation, compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

Also contemplated herein is pulmonary delivery of the compounds disclosed herein (or salts thereof). The compound is delivered to the lungs of a mammal while inhaling and traverses across the lung epithelial lining to the blood stream. Other reports of inhaled molecules include Adjei et al., Pharm Res 7:565-569 (1990); Adjei et al., Int J Pharmaceutics 63:135-144 (1990) (leuprolide acetate); Braquet et al., J Cardiovasc Pharmacol 13(suppl. 5):143-146 (1989) (endothelin-1); Hubbard et al., Annal Int Med 3:206-212 (1989) (α1-antitrypsin); Smith et al., 1989, J Clin Invest 84:1145-1146 (a-1-proteinase); Oswein et al., 1990, “Aerosolization of Proteins”, Proceedings of Symposium on Respiratory Drug Delivery II, Keystone, Colo., March, (recombinant human growth hormone); Debs et al., 1988, J Immunol 140:3482-3488 (interferon-gamma and tumor necrosis factor alpha) and Platz et al., U.S. Pat. No. 5,284,656 (granulocyte colony stimulating factor; incorporated by reference). A method and composition for pulmonary delivery of drugs for systemic effect is described in U.S. Pat. No. 5,451,569 (incorporated by reference), issued Sep. 19, 1995 to Wong et al.

Contemplated for use in the practice of this invention are a wide range of mechanical devices designed for pulmonary delivery of therapeutic products, including but not limited to nebulizers, metered dose inhalers, and powder inhalers, all of which are familiar to those skilled in the art.

Some specific examples of commercially available devices suitable for the practice of this invention are the Ultravent nebulizer, manufactured by Mallinckrodt, Inc., St. Louis, Mo.; the Acorn II nebulizer, manufactured by Marquest Medical Products, Englewood, Colo.; the Ventolin metered dose inhaler, manufactured by Glaxo Inc., Research Triangle Park, N.C.; and the Spinhaler powder inhaler, manufactured by Fisons Corp., Bedford, Mass.

All such devices require the use of formulations suitable for the dispensing of the compounds of the invention. Typically, each formulation is specific to the type of device employed and may involve the use of an appropriate propellant material, in addition to the usual diluents, adjuvants and/or carriers useful in therapy. Also, the use of liposomes, microcapsules or microspheres, inclusion complexes, or other types of carriers is contemplated. Chemically modified compound of the invention may also be prepared in different formulations depending on the type of chemical modification or the type of device employed.

Formulations suitable for use with a nebulizer, either jet or ultrasonic, will typically comprise a compound of the invention (or derivative) dissolved in water at a concentration of about 0.1 to 25 mg of biologically active compound of the invention per mL of solution. The formulation may also include a buffer and a simple sugar (e.g., for inhibitor stabilization and regulation of osmotic pressure). The nebulizer formulation may also contain a surfactant, to reduce or prevent surface induced aggregation of the compound of the invention caused by atomization of the solution in forming the aerosol.

Formulations for use with a metered-dose inhaler device will generally comprise a finely divided powder containing the compound of the invention (or derivative) suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

Formulations for dispensing from a powder inhaler device will comprise a finely divided dry powder containing compound of the invention (or derivative) and may also include a bulking agent, such as lactose, sorbitol, sucrose, or mannitol in amounts which facilitate dispersal of the powder from the device, e.g., 50 to 90% by weight of the formulation. The compound of the invention (or derivative) should advantageously be prepared in particulate form with an average particle size of less than 10 micrometers (m), most preferably 0.5 to 5 μm, for most effective delivery to the deep lung.

Nasal delivery of a pharmaceutical composition of the present invention is also contemplated. Nasal delivery allows the passage of a pharmaceutical composition of the present invention to the blood stream directly after administering the therapeutic product to the nose, without the necessity for deposition of the product in the lung. Formulations for nasal delivery include those with dextran or cyclodextran.

For nasal administration, a useful device is a small, hard bottle to which a metered dose sprayer is attached. In one embodiment, the metered dose is delivered by drawing the pharmaceutical composition of the present invention solution into a chamber of defined volume, which chamber has an aperture dimensioned to aerosolize and aerosol formulation by forming a spray when a liquid in the chamber is compressed. The chamber is compressed to administer the pharmaceutical composition of the present invention. In a specific embodiment, the chamber is a piston arrangement. Such devices are commercially available.

Alternatively, a plastic squeeze bottle with an aperture or opening dimensioned to aerosolize an aerosol formulation by forming a spray when squeezed is used. The opening is usually found in the top of the bottle, and the top is generally tapered to partially fit in the nasal passages for efficient administration of the aerosol formulation. Preferably, the nasal inhaler will provide a metered amount of the aerosol formulation, for administration of a measured dose of the drug.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethylcellulose, sorbitol, or dextran. Optionally, the suspension may also contain suitable stabilizers or agents which increase the solubility of the compounds to allow for the preparation of highly concentrated solutions.

Alternatively, the active compounds may be in powder form for constitution with a suitable vehicle, e.g., sterile pyrogen-free water, before use.

The compounds may also be formulated in rectal or vaginal compositions such as suppositories or retention enemas, e.g., containing conventional suppository bases such as cocoa butter or other glycerides.

In addition to the formulations described above, a compound may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer R, Science 249:1527-33 (1990).

The compound of the invention and optionally other therapeutics may be administered per se (neat) or in the form of a pharmaceutically acceptable salt or cocrystal. When used in medicine the salts or cocrystals should be pharmaceutically acceptable, but non-pharmaceutically acceptable salts or cocrystals may conveniently be used to prepare pharmaceutically acceptable salts or cocrystals thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

Pharmaceutical compositions of the invention contain an effective amount of a compound as described herein and optionally therapeutic agents included in a pharmaceutically acceptable carrier. The term “pharmaceutically acceptable carrier” means one or more compatible solid or liquid filler, diluents or encapsulating substances which are suitable for administration to a human or other vertebrate animal. The term “carrier” denotes an organic or inorganic ingredient, natural or synthetic, with which the active ingredient is combined to facilitate the application. The components of the pharmaceutical compositions also are capable of being commingled with the compounds of the present invention, and with each other, in a manner such that there is no interaction which would substantially impair the desired pharmaceutical efficiency.

The therapeutic agent(s), including specifically but not limited to a compound of the invention, may be provided in particles. Particles as used herein means nanoparticles or microparticles (or in some instances larger particles) which can consist in whole or in part of the compound of the invention or the other therapeutic agent(s) as described herein. The particles may contain the therapeutic agent(s) in a core surrounded by a coating, including, but not limited to, an enteric coating. The therapeutic agent(s) also may be dispersed throughout the particles. The therapeutic agent(s) also may be adsorbed into the particles. The particles may be of any order release kinetics, including zero-order release, first-order release, second-order release, delayed release, sustained release, immediate release, and any combination thereof, etc. The particle may include, in addition to the therapeutic agent(s), any of those materials routinely used in the art of pharmacy and medicine, including, but not limited to, erodible, nonerodible, biodegradable, or nonbiodegradable material or combinations thereof. The particles may be microcapsules which contain the compound of the invention in a solution or in a semi-solid state. The particles may be of virtually any shape.

Both non-biodegradable and biodegradable polymeric materials can be used in the manufacture of particles for delivering the therapeutic agent(s). Such polymers may be natural or synthetic polymers. The polymer is selected based on the period of time over which release is desired. Bioadhesive polymers of particular interest include bioerodible hydrogels described in Sawhney H S et al. (1993) Macromolecules 26:581-7, the teachings of which are incorporated herein. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

The therapeutic agent(s) may be contained in controlled release systems. The term “controlled release” is intended to refer to any drug-containing formulation in which the manner and profile of drug release from the formulation are controlled. This refers to immediate as well as non-immediate release formulations, with non-immediate release formulations including but not limited to sustained release and delayed release formulations. The term “sustained release” (also referred to as “extended release”) is used in its conventional sense to refer to a drug formulation that provides for gradual release of a drug over an extended period of time, and that preferably, although not necessarily, results in substantially constant blood levels of a drug over an extended time period. The term “delayed release” is used in its conventional sense to refer to a drug formulation in which there is a time delay between administration of the formulation and the release of the drug there from. “Delayed release” may or may not involve gradual release of drug over an extended period of time, and thus may or may not be “sustained release.”

Use of a long-term sustained release implant may be particularly suitable for treatment of chronic conditions. “Long-term” release, as used herein, means that the implant is constructed and arranged to deliver therapeutic levels of the active ingredient for at least 7 days, and preferably 30-60 days. Long-term sustained release implants are well-known to those of ordinary skill in the art and include some of the release systems described above.

It will be understood by one of ordinary skill in the relevant arts that other suitable modifications and adaptations to the compositions and methods described herein are readily apparent from the description of the invention contained herein in view of information known to the ordinarily skilled artisan, and may be made without departing from the scope of the invention or any embodiment thereof. Having now described the present invention in detail, the same will be more clearly understood by reference to the following examples, which are included herewith for purposes of illustration only and are not intended to be limiting of the invention.

### EXAMPLES

The invention is further described in the following examples, which do not limit the scope of the invention described in the claims.

### Example 1: PathHunter MEM-EA Pharmacotrafficking Assay for SLC6A8 CTD Mutants Cell Lines

### Preparation of Cells

U-2 OS MEM-EA cells were purchased from Eurofins (catalog #93-1101C3). From these parental cells, stable cell lines expressing SLC6A8 CTD mutants were made using standard cell culture protocols, involving transfections of plasmids followed by antibiotic selection. These plasmids encoded CTD mutant SLC6A8 proteins with a C-terminal ProLink2 tag. U-2 OS MEM-EA cells and derived stable cell lines were grown in RPMI medium 1640 (Thermo Fisher Scientific, catalog #A10491-01) supplemented with 10% Fetal Bovine Serum (FBS), 200 ug/mL hygromycin B (Thermo Fisher Scientific, catalog #10687010), 100 mg/mL streptomycin, and 100 U/mL penicillin. Cells were grown at 37° C. in a humidified CO₂ incubator.

### Assay

U-2 OS MEM-EA cells stably expressing SLC6A8 CTD mutants were plated into white-walled 96-well plates (Corning, catalog #3903) at a density of 20,000 cells per well. For background subtraction, the parental U-2 OS MEM-EA cells were also plated. After 24 hrs, compounds were dispensed directly into the plated cells using the Tecan D300e Digital Dispenser. After an additional 24 hrs, the media with compound was again removed and white covers (Thermo Fisher Scientific, catalog #236272) were placed on the bottoms of the 96-well plates. Luminescence indicative of SLC6A8 CTD mutant cell surface localization was measured according to the manufacturer's protocol, using the PathHunter Detection kit (Eurofins catalog #93-0001L) and an EnVision plate reader (PerkinElmer, 2104 multilabel reader). Data were analyzed in Excel. Background signal from wells containing parental cells was subtracted, and then fold-changes were computed with respect to DMSO.

### Example 2: Corrector Assay for SLC6A8 Mutant Cell Lines

### Preparation of Cells

A number of SLC6A8 CTD mutant cell lines were made in U-2 OS MEM-EA cells, 293T cells, HeLa cells, and CHO cells. All cells lines were generated as described above for U-2 OS MEM-EA cells, namely stable cell lines expressing SLC6A8 CTD mutants were made using standard cell culture protocols involving transfections of plasmids followed by antibiotic selection.

### Assay

Stable cell lines expressing CTD mutants were plated into 96-well plates (Corning, catalog #3595) at a density of 40,000 cells per well. After 24 hrs, compounds were dispensed directly into the plated cells using a Tecan D300e Digital Dispenser.

After an additional 24 hrs, the media with compound was removed. Cells were then incubated with a solution of 100 uM D3-creatine (SIGMA, 616249-1G) in media (without FBS). This solution was incubated with the cells for a 30 min incubation at 37° C. After the incubation, the media was removed, and the cells were washed once with 180 uL of phosphate buffered solution (PBS). To extract metabolites, water was added to the cells for 1 hour with vigorous shaking at 700 rpm. Cell extracts were analyzed on an ABSciex-4000 triple quad mass spectrometer coupled with a RapidFire sample desalting/injection system with a graphitic carbon desalting column and a basic buffer system in reverse phase. Abundances of D3-creatine were analyzed in Excel, and then fold-changes were computed with respect to DMSO.

### Example 3: General Procedures for the Synthesis of Representative Compounds of the Invention

### Synthesis of Acylguanidines

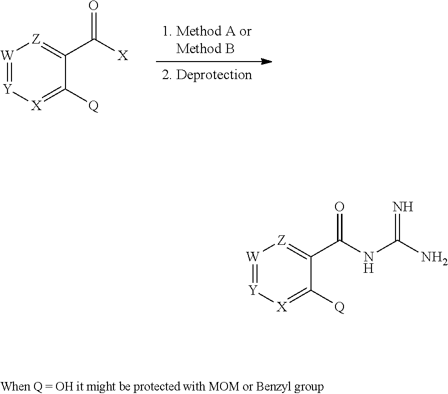

### General Procedure A

Step 1—When X=OMe; Method A: guanidine hydrochloride, t-BuOK, DMF, RT

When X=OH; Method B: Boc-guanidine, NMM, BOP, DMF, RT

### Step 2

When Q=OMOM and guanidine is Boc-protected—TFA, DCM, RT
When Q=OBn and guanidine is Boc-protected—H₂, Pd/C, EtOAc, RT, then TFA, DCM, RT

### Step 1—Method A

To a suspension of guanidine hydrochloride (10 equiv.) in dry DMF (0.5M) t-BuOK (8 equiv.) was added under argon atmosphere. The resulting mixture was stirred at RT for 45 min, then a solution of the appropriate ester intermediate (1 equiv.) in dry DMF (0.8M) was added and the reaction was stirred at RT until completion. The reaction mixture was diluted with EtOAc and the resulting mixture was washed with saturated NH₄Cl and brine, dried over Na₂SO₄, filtered and concentrated under vacuum. The crude product is then purified by flash chromatography or prep HPLC.

### Step 1—Method B

To a mixture of the appropriate carboxylic acid intermediate (1 equiv.) and Boc-guanidine (2 equiv.) in dry DMF (0.2M), N-Methylmorpholine (4 equiv.) and BOP (1.5 equiv.) were added. The resulting mixture was stirred at RT for several hours until completion. The reaction mixture was diluted with saturated NH₄Cl and extracted with EtOAc (×3). The combined organic layers were dried over Na₂SO₄, filtered and concentrated under vacuum. The crude product is then purified by flash chromatography or prep HPLC.

### Step 2—MOM and Boc Deprotection

To a solution of acylguanidine (1 equiv.) in DCM (0.1M) TFA (40 equiv.) was added at 0° C. and the reaction was slowly warmed to RT and stirred until completion. The solvent was removed under reduced pressure and the crude was purified by prep HPLC.

### Step 2—Benzyl Deprotection Followed by Boc Deprotection

To a solution of acylguanidine (1 equiv.) in Ethyl Acetate Pd 10% on carbon (0.1 equiv.) was added and reaction stirred under hydrogen until completion. The reaction mixture was then filtered through Celite and the solvent was removed under reduced pressure. The crude intermediate was then dissolved in DCM (0.1M) and TFA (20 equiv.) was added at 0° C. and the reaction was slowly warmed to RT and stirred until completion. The solvent was removed under reduced pressure and the crude was purified by prep HPLC.

### General Procedure B

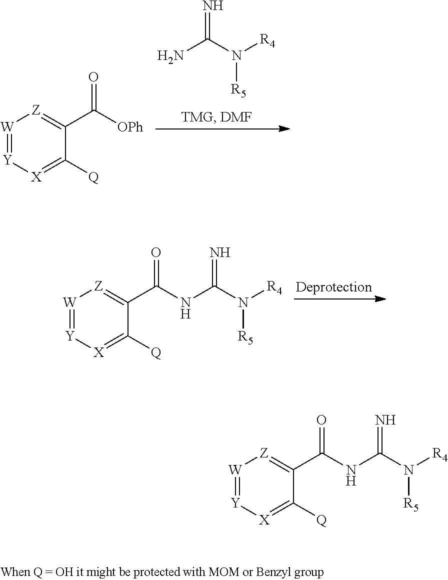

### Step 1

To a solution of the appropriate phenylester intermediate (1 equiv.) in DMF (0.2M) 1,1,3,3-Tetramethylguanidine (1.5 equiv.) was added followed by mono- or bis-substituted guanidine (2 equiv.) and the resulting reaction mixture was stirred at RT for several hours until completion. The reaction mixture was then diluted with water and extracted with EtOAc (×3). The combined organic layers were dried over Na₂SO₄, filtered and concentrated under vacuum. The crude product was then purified by prep HPLC.

Step 2—See deprotection steps in General Procedure A of acylguanidine synthesis.

### General Procedure C

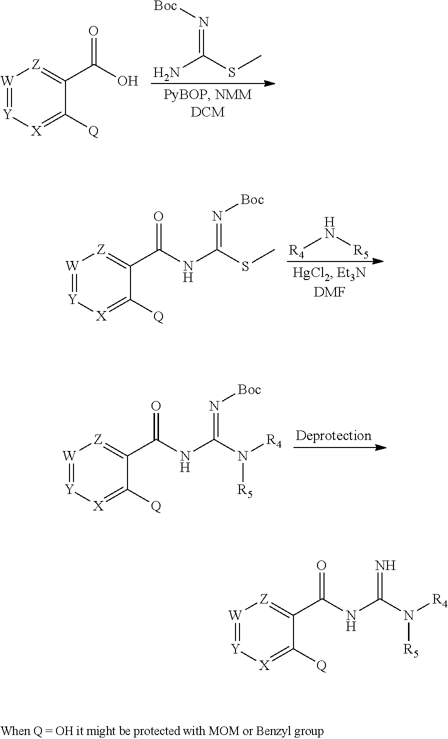

### Step 1

The carboxylic acid intermediate (1 equiv.) was dissolved in dry DMF (0.2M), then NMM (4 equiv.) and PyBOP (1.5 equiv.) were added followed by tert-butyl N-[(methylsulfanyl) methaninidoyl]carbamate (1.1 eq). The reaction was stirred at RT until completion. The reaction mixture was then diluted with EtOAc and washed with saturated NH₄Cl and brine. The organic solution was then dried over Na₂SO₄, filtered and concentrated under vacuum. The crude product was then purified by flash chromatography.

### Step 2

The above intermediate (1 equiv.) was then dissolved in dry DMF (0.2M) and Et₃N (10 equiv.) was added followed by HgCl₂ (1 equiv.) and the appropriate mono- or bis-substituted amine (1 eq). The reaction was stirred at RT until completion. The reaction mixture was then diluted with EtOAc and filtered through Celite. The filtrate was washed with saturated NH₄Cl and brine. The organic solution was then dried over Na₂SO₄, filtered and concentrated under vacuum. The crude product was then purified by flash chromatography.

Step 3—See deprotection steps in General Procedure A of acylguanidine synthesis.

### General Procedure D

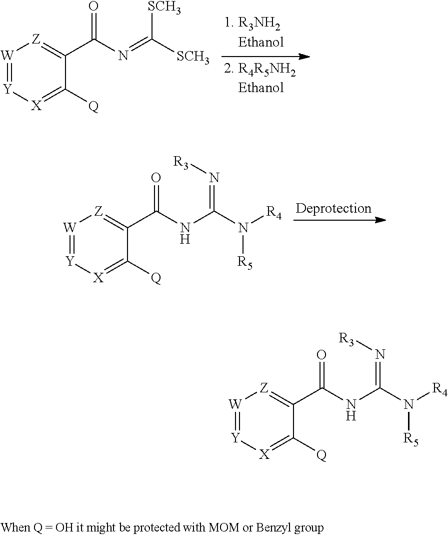

### Step 1

The appropriate S,S-dimethyl N-aroylcarbimidodithiolate intermediate (1 equiv.) was dissolved in Ethanol (0.2M) and the appropriate primary amine (1 equiv.) was added. The reaction was stirred at RT until completion. Then the solvent was evaporated under vacuum and the crude intermediate was then reacted in Ethanol (0.2M) with the appropriate mono- or bis-substituted amine (1.5 eq). The reaction was stirred at RT until completion. The reaction mixture was diluted with water and extracted with EtOAc (×3). The combined organic layers were dried over Na₂SO₄, filtered and concentrated under vacuum. The crude was then purified by prep HPLC.

Step 2—See deprotection steps in General Procedure A of acylguanidine synthesis.

### General Procedure E

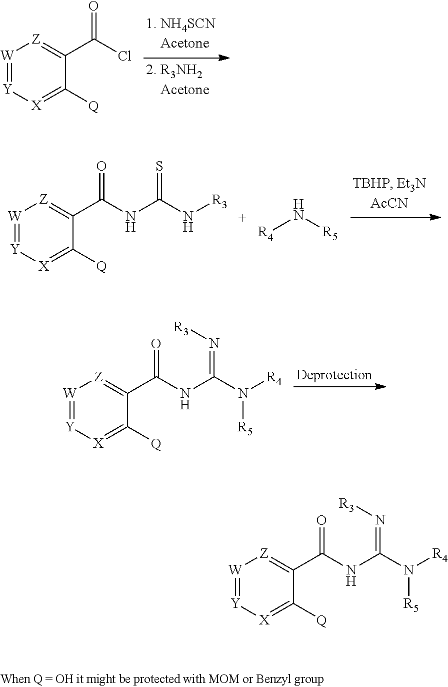

### Step 1

Suitable benzoyl chloride (1 eq, 11 mmol) was added to ammonium thiocyanate (1 equiv.) in acetone (0.4M). The reaction mixture was refluxed for 15 min and then cooled down to RT. An acetone solution of the appropriate primary amine (1 equiv.) was added and reaction refluxed for further 30 min or at RT for 3 hours. The reaction mixture was then poured into crushed ice and the resulting mixture was rigorously stirred. The solid was then filtered off and washed with water and used as crude for next step.

### Step 2

In a round bottomed flask charged with a solution of the crude benzoylthioureia (1 equiv.) in acetonitrile (0.1M) under vigorous magnetic stirring and cooled to 0° C. were added, respectively, trimethylamine (1 equiv.), the suitable nucleophilic mono- or bis-substituted amine (1 equiv.) and 70% aqueous tert-butyl hydroperoxide (3 eq). The reaction was slowly warmed to RT and after consumption of starting material the mixture was transferred to a separatory funnel charged with a NaHSO₃ saturated solution and extracted with DCM (×3). The combined organic layers were dried over Na₂SO₄, filtered and concentrated under reduced pressure. The crude was then purified by prep HPLC.

Step 3—See deprotection steps in General Procedure A of acylguanidine synthesis.

### General Procedure F

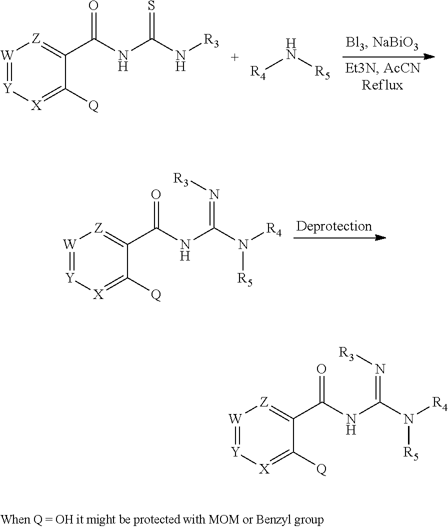

### Step 1

To a solution of the appropriate thiourea (1 eq, synthesized as in step 1 of general procedure E) in CH₃CN (0.1M) were added the appropriate mono- or bis-substituted amine (1 equiv.) and Et₃N (2 equiv.), followed by NaBiO₃ (1 equiv.) and BiI₃ (0.5 eq). The suspension was left stirring for 10 min at RT and became black. After this time, reaction was refluxed until completion. The solvent was then evaporated and DCM was added. The suspension was filtered through a pad of Celite and the pad washed twice with DCM. The filtrate was dried over anhydrous Na₂SO₄, filtered and the solvent was evaporated under vacuum. The crude product was purified by prep HPLC.

Step 2—See deprotection steps in General Procedure A of acylguanidine synthesis.

### General Procedure G

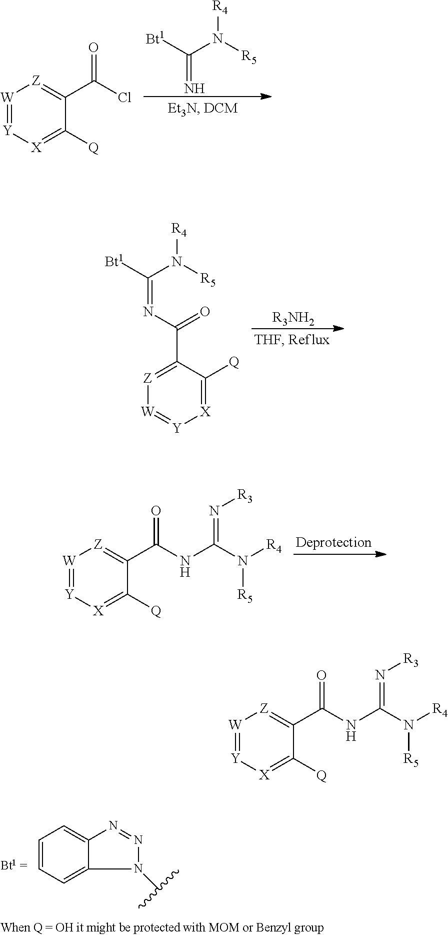

### Step 1

To a stirred solution of appropriate 1H-benzotriazol-1-ylcarboximidamide derivative (1 equiv.) in DCM (0.3M), the appropriate acid chloride (1 equiv.) was added at RT followed by the addition of triethylamine (1 eq). The reaction mixture was stirred at RT overnight. Upon completion, the reaction mixture was washed twice with water, dried over Na₂SO₄, filtered and concentrated under vacuum. The crude product was then purified by flash chromatography.

### Step 2

To a solution of N-acyl-1H-benzotriazol-1-ylcarboximidamide derivatives (1 equiv.) in THF (0.1M), the amine of choice (1 equiv.) was added. The reaction mixture was then refluxed until full conversion of starting materials. Upon completion, the solvent was evaporated under reduced pressure and the crude product was dissolved in DCM, washed twice with saturated aqueous sodium carbonate, dried over Na₂SO₄, and filtered. The solvent was removed under reduced pressure and the crude was purified by prep HPLC.

Step 3—See deprotection steps in General Procedure A of acylguanidine synthesis.

### Synthesis of Thiolylguanidines

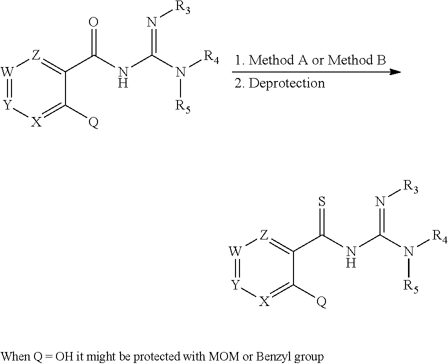

### Step 1—Method A

The appropriate acylguanidine (1 equiv.) was dissolved in Toluene (0.05M), then Lawesson's reagent was added (1 equiv.) and reaction refluxed until completion. Solvent was then removed under vacuum and crude was purified by prep HPLC.

### Step 1—Method B

The appropriate acylguanidine (1 equiv.) was dissolved in pyridine and phosphorus pentasulfide (1 equiv.) was added and reaction refluxed until completion. The reaction was then cooled down and poored into ice water. The resulting mixture was then extracted with EtOAc (×3) and the combined organic layers were dried over Na₂SO₄, filtered and concentrated under vacuum. The crude was then purified by prep HPLC

Step 2—See deprotection steps in General Procedure A of acylguanidine synthesis.

### Synthesis of Cyclic Acylguanidines

### General Procedure A

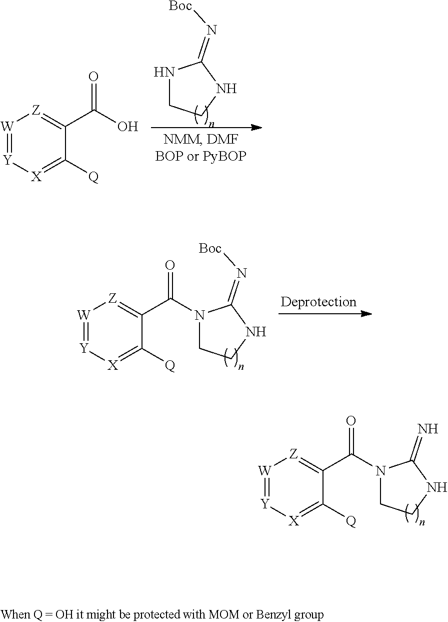

### Step 1

To a mixture of the appropriate carboxylic acid intermediate (1 equiv.) and the appropriate Boc-cyclicguanidine (2 equiv.) in dry DMF (0.2M)N-Methylmorpholine (4 equiv.) and BOP or PyBOP (1.5 equiv.) were added. The resulting mixture was stirred at RT for several hours until completion. The reaction mixture was diluted with saturated NH₄Cl and extracted with EtOAc (×3). The combined organic layers were dried over Na₂SO₄, filtered and concentrated under vacuum. The crude product was then purified by column chromatography or prep HPLC.

Step 2—See deprotection steps in General Procedure A of acylguanidine synthesis.

### General Procedure B

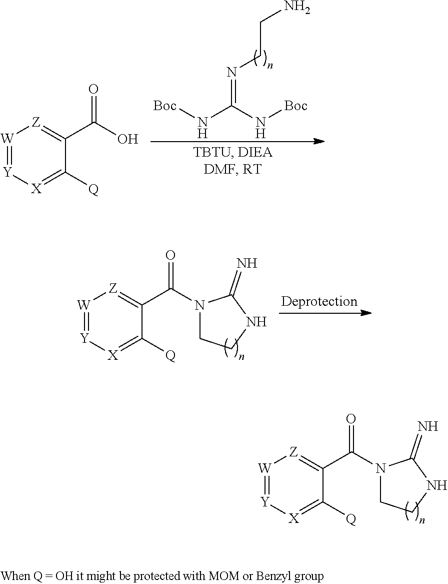

### Step 1

To a solution of the appropriate carboxylic acid intermediate (1 equiv.) and TBTU (1.5 equiv.) was added the appropriate di-Boc-guanidine (1.5 equiv.) and DIEA (2.5 eq). Reaction was stirred at RT until completion. The crude was purified by prep HPLC.

Step 2—See deprotection steps in General Procedure A of acylguanidine synthesis.

### General Procedure C

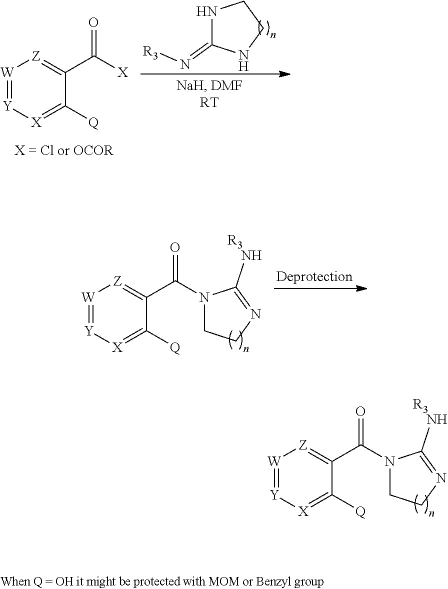

### Step 1

The appropriate cyclic guanidine (1 equiv.) was dissolved in DMF (0.1M) then NaH (2.5 equiv.) was added and reaction stirred at RT for 30 min before the appropriate acyl chloride or anhydride (2.5 equiv.) was added. Reaction stirred at RT until completion. The reaction mixture was diluted with water and extracted with DCM (×3). The combined organic layers were dried over Na₂SO₄, filtered and concentrated under vacuum. The crude product was purified by prep HPLC.

Step 2—See deprotection steps in General Procedure A of acylguanidine synthesis.

### General Procedure D

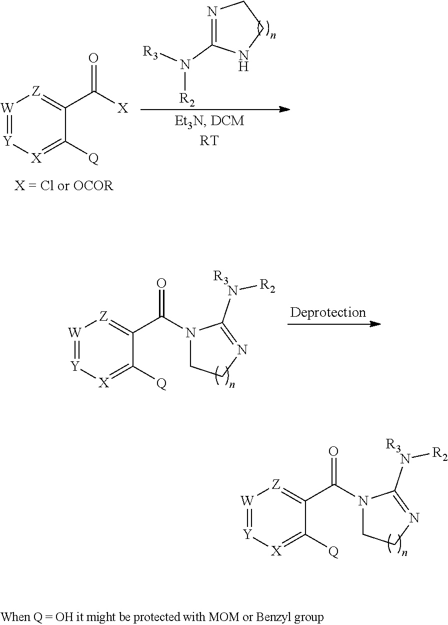

### Step 1

To a solution of the appropriate cyclic guanidine (1 equiv.) and triethylamine (1.5 equiv.) in DCM (0.1M) the appropriate acyl chloride or anhydride (1 equiv.) was added at 0° C. and reaction was slowly warmed to RT and stirred until completion. The reaction mixture was then washed with water, dried over Na₂SO₄, filtered and concentrated under vacuum. The crude product was purified by prep HPLC.

Step 2—See deprotection steps in General Procedure A of acylguanidine synthesis.

### General Procedure E

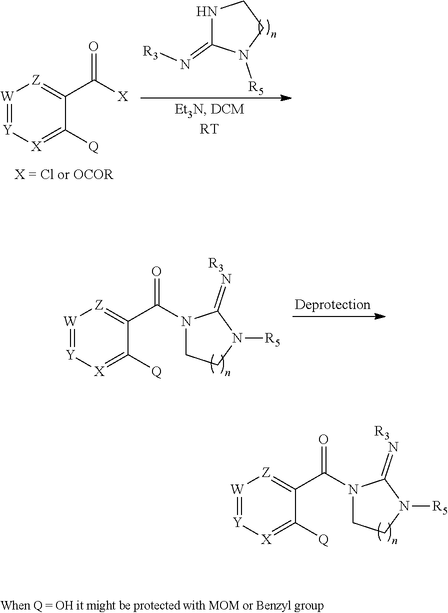

### Step 1

To a solution of the appropriate cyclic guanidine (1 equiv.) and triethylamine (1.5 equiv.) in DCM (0.1M) the appropriate acyl chloride or anhydride (1 equiv.) was added at 0° C. and reaction was slowly warmed to RT and stirred until completion. The reaction mixture was then washed with water, dried over dried over Na₂SO₄, filtered and concentrated under vacuum. The crude product was purified by prep HPLC.

Step 2—See deprotection steps in General Procedure A of acylguanidine synthesis.

### General Procedure F

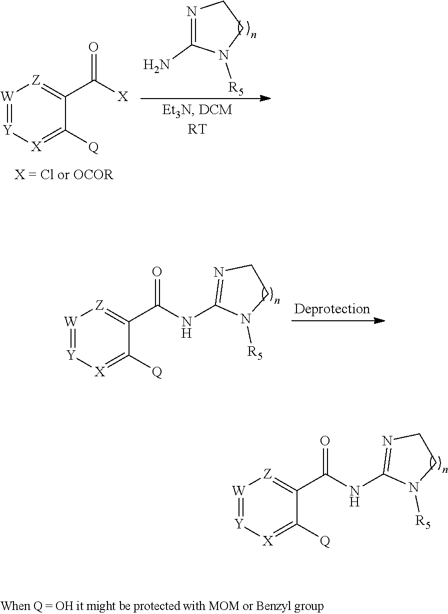

### Step 1

To a solution of the appropriate cyclic guanidine (1 equiv.) and triethylamine (1.5 equiv.) in DCM (0.1M) the appropriate acyl chloride or anhydride (1 equiv.) was added at 0° C. and reaction was slowly warmed to RT and stirred until completion. The reaction mixture was then washed with water, dried over dried over Na₂SO₄, filtered and concentrated under vacuum. The crude product was purified by prep HPLC.

Step 2—See deprotection steps in General Procedure A of acylguanidine synthesis.

### Synthesis of Cyclic Thiolylguanidines

The synthesis of cyclic thiolylguanidines is performed starting from cyclic acylguanidines, whose synthesis is described in the section above, using the reaction conditions described in the synthesis of thiolylguanidines section.

### Synthesis of Sulfaguanidines

### General Procedure A

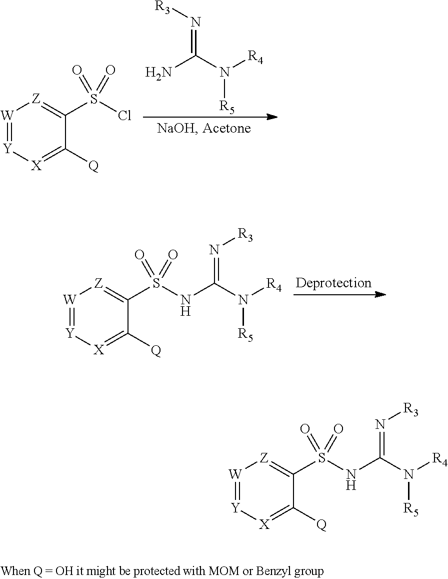

### Step 1

The appropriate guanidine (1 equiv.) was dissolved in aqueous NaOH and stirred at RT for 45-60 min, then Acetone was added (0.1M) and the reaction mixture was cooled to 0° C. and the appropriate sulfonyl chloride (1 equiv.) was added and reaction was slowly warmed to RT and stirred until completion. 1 HCl was added to bring the pH to acidic and then the mixture was extracted with DCM (×3). The combined organic layers were dried over Na₂SO₄, filtered and concentrated under vacuum. The crude product was purified by prep HPLC.

Step 2—See deprotection steps in General Procedure A of acylguanidine synthesis.

### General Procedure B

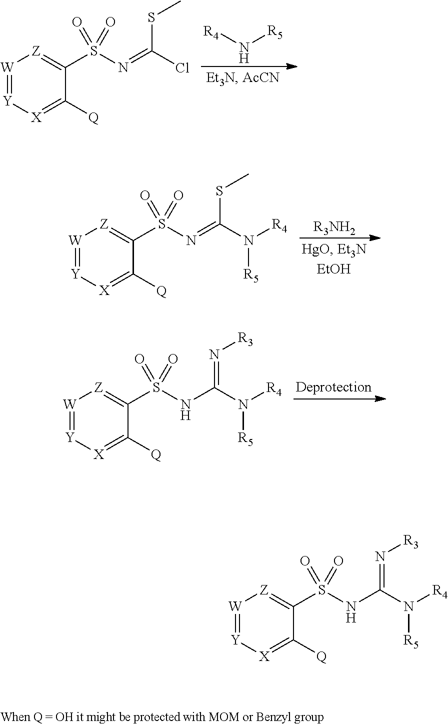

### Step 1

A flame dried flask equipped with a stirbar was cooled under a stream of nitrogen and charged with the appropriate carbonochloridoimidothioate (1 equiv.) and anhydrous acetonitrile (0.15 M). The mixture was cooled to 0° C. then triethylamine (1.2) was added dropwise. The appropriate amine (1.1 equiv.) was then added dropwise as a solution in acetonitrile (1.5 mL/mmol of amine). The reaction mixture was then warmed to RT and stirred until completion. The solvent was removed under reduced pressure, and the crude product was purified by flash chromatography or prep HPLC.

### Step 2

A flame-dried round bottom flask equipped with a stir bar was cooled under a stream of nitrogen and charged with the above intermediate (1 equiv.), mercuric oxide (1.5 eqs), and triethylamine (4.5 equiv.), followed by the appropriate amine (3 to 5 equiv.) and the mixture was stirred at RT until completion. The solution was then filtered through Celite, and the solvent was removed under reduced pressure. The crude product was purified by prep HPLC.

Step 3—See deprotection steps in General Procedure A of acylguanidine synthesis.

### General Procedure C

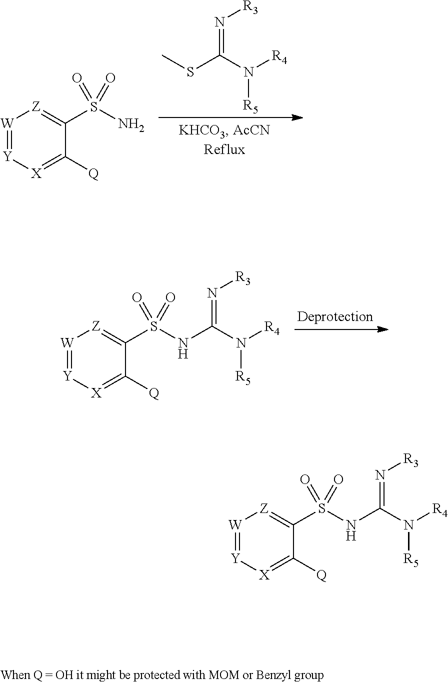

### Step 1

To a solution of the appropriate thiomethyl derivative (1 equiv.) in acetonitrile (0.2M), KHCO₃ (1.5 equiv.) and the appropriate sulfonamide (1.1 equiv.) was added and the reaction was refluxed until completion. The reaction mixture was diluted with water and the resulting mixture was extracted with DCM (×3). The combined organic layers were dried over Na₂SO₄, filtered and concentrated under vacuum. The crude product was purified by prep HPLC.

Step 2—See deprotection steps in General Procedure A of acylguanidine synthesis.

### Synthesis of Cyclic Sulfaguanidines

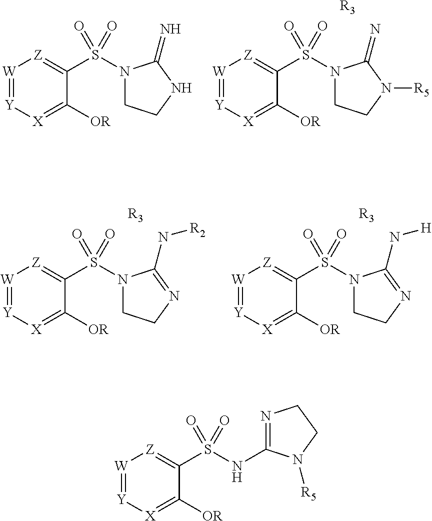

These final compounds are prepared using the same reaction conditions than the cyclic acyllguanidines starting from the appropriate sulfonyl chlorides and cyclic guanidines.

### Synthesis of Guanidines

### General Procedure A

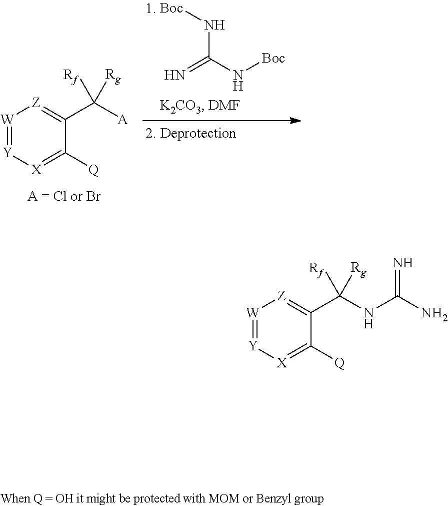

### Step 1

To a solution of Boc-protected guanidine (1.2 equiv.) in DMF (0.2M) potassium carbonate (1.5 equiv.) was added followed by the appropriate halide (1 equiv.) and reaction stirred at RT until completion. The reaction mixture was diluted with water and the resulting mixture was extracted with DCM (×3). The combined organic layers were dried Na₂SO₄, filtered and concentrated under vacuum. The crude product was purified by prep HPLC.

Step 2—See deprotection steps in General Procedure A of acylguanidine synthesis.

### General Procedure B

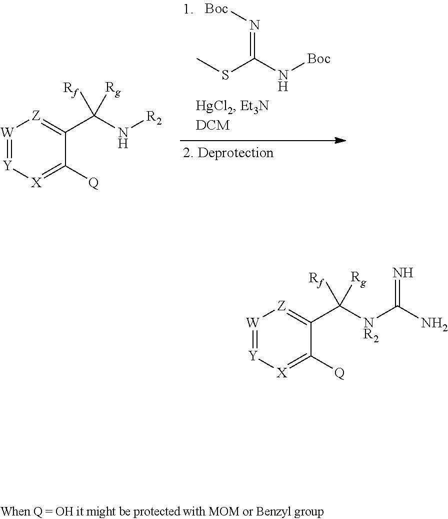

### Step 1

To an ice-cooled solution of the appropriate amine (1.5 equiv.) in DCM (0.15M) was added sequentially triethylamine (4.5 equiv.), 1,3-bis(ter/-butoxycarbonyl)-2-methyl-2-thiopseudourea (1 equiv.) and HgCl₂ (1 eq). The reaction was slowly warmed to RT and stirred until completion. The suspension was filtered through a plug of Celite and the filter-cake was washed with further DCM. The filtrate was washed sequentially with 10% aqueous citric acid, 10% aqueous potassium carbonate and brine. The organic phase was dried over Na₂SO₄, filtered and concentrated under vacuum. The crude product was purified by prep HPLC.

Step 2—See deprotection steps in General Procedure A of acylguanidine synthesis.

### General Procedure C

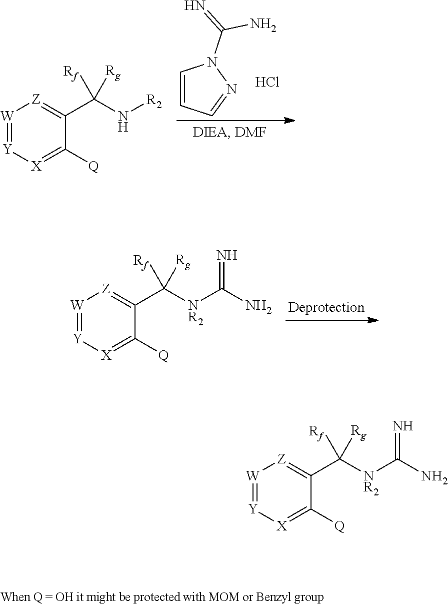

### Step 1

The appropriate amine (1 equiv.) was dissolved in a mixture of anhydrous DMF (0.3M) and DIPEA (8 eq). 1H-pyrazole-1-carboxamidine hydrochloride (2 equiv.) was then added, and the reaction mixture was stirred at RT until completion. Water was added and the resulting mixture was extracted with DCM (×3). The combined organic layers were dried over Na₂SO₄, filtered and concentrated under vacuum. The crude product was purified by prep HPLC.

Step 2—See deprotection steps in General Procedure A of acylguanidine synthesis.

### General Procedure D

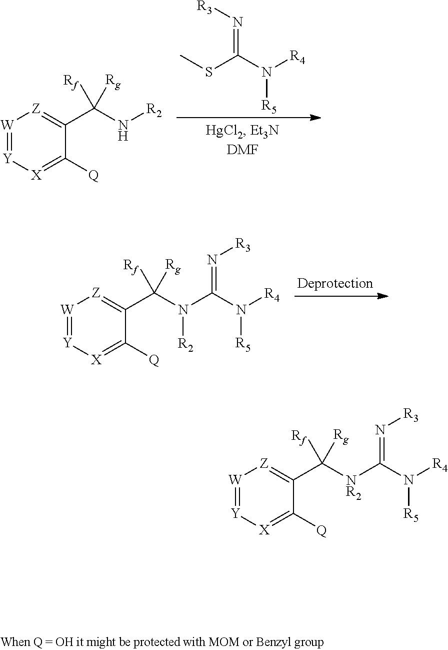

### Step 1

The appropriate amine (1 equiv.) was dissolved in dry DMF and Et₃N (10 equiv.) was added followed by HgCl₂ (1 equiv.) and the appropriate thiomethyl derivative (1 eq). The reaction was stirred at RT until completion. The reaction mixture was then diluted with EtOAc and filtered through Celite. The filtrate was washed with saturated NH₄Cl and brine. The organic solution was then dried over Na₂SO₄, filtered and concentrated under vacuum. The crude product was then purified by prep HPLC.

Step 2—See deprotection steps in General Procedure A of acylguanidine synthesis.

### General Procedure E

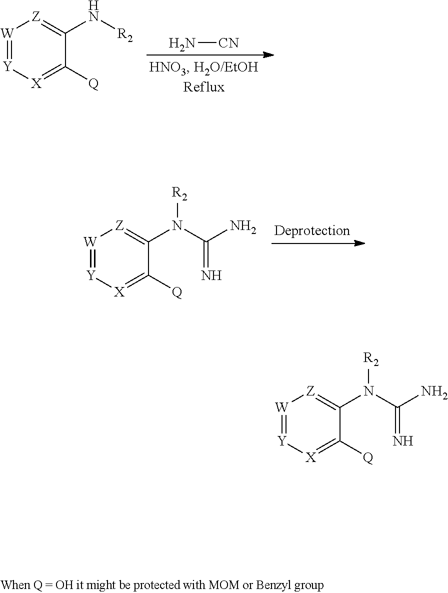

### Step 1

Nitric acid (69%) was added dropwise to a solution of the appropriate aniline derivative (1 equiv.) in EtOH (0.2M), followed by addition of a solution of cyanamide (5 equiv.) in a minimal amount of H₂O. The reaction mixture was heated at reflux for 18-36 hours, and then concentrated under vacuum. The crude product was purified by prep HPLC.

Step 2—See deprotection steps in General Procedure A of acylguanidine synthesis.

### General Procedure F

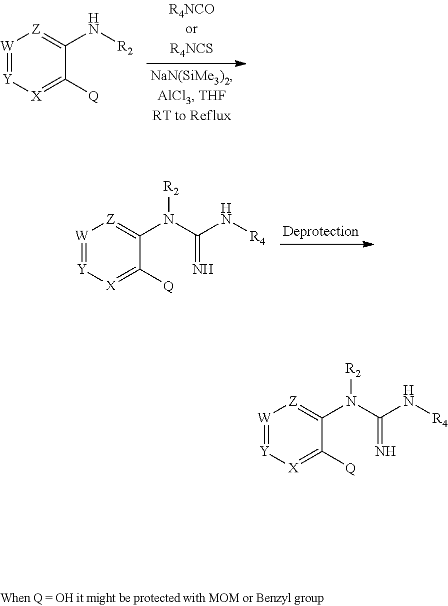

### Step 1

Isocyanates or isothiocyanates (1 equiv.) and sodium bis(trimethylsilyl)amide (2.0 M in THF, 1.2 equiv.) were added into a two-necked flask at RT and reaction stirred under nitrogen for 1 h.

After isocyanates or isothiocyanates were completely consumed and converted to the cyanamide anion intermediates, various appropriate aniline (2.2 equiv.), AlCl₃, (0.1 eq, 10% w/w)) were added and reaction heated at reflux for 6-12 h under N₂. After the reaction was completed, the reaction mixture was filtrated, washed with DCM and concentrated under reduced pressure. The residue was purified by prep HPLC.

Step 2—See deprotection steps in General Procedure A of acylguanidine synthesis.

### General Procedure G

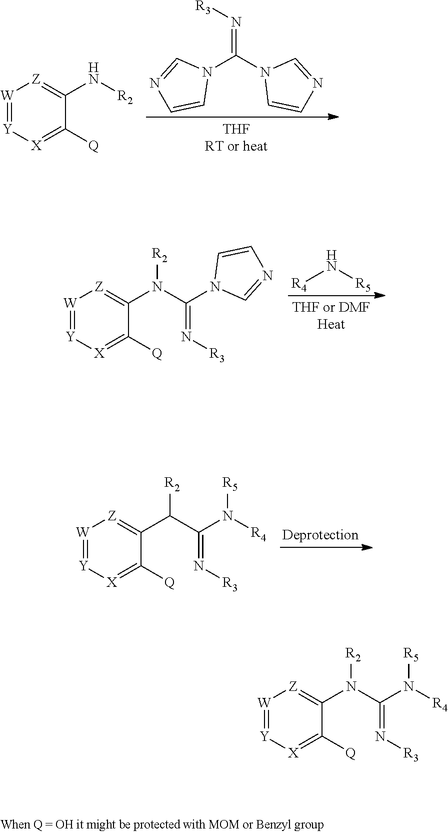

### Step 1

To a solution of the appropriate diimidazole derivative (1 equiv.) in THF (0.4M) the appropriate amine (1.2 equiv.) was added and reaction stirred at RT or 40° C. until completion. Water was then added and the resulting mixture was extracted with DCM (×3). The combined organic layers were dried over Na₂SO₄, filtered and concentrated under vacuum. The crude product was used for next step without any further purification.

### Step 2

The above intermediate (1 equiv.) was dissolved in THF or DMF (0.5M) and the appropriate amine (1.5 equiv.) was added and reaction heated until completion. Water was then added and the resulting mixture was extracted with DCM (×3). The combined organic layers were dried over Na₂SO₄, filtered and concentrated under vacuum. The crude product was purified by prep HPLC.

Step 3—See deprotection steps in General Procedure A of acylguanidine synthesis.

### Synthesis of Cyclic Guanidines

General Procedure A

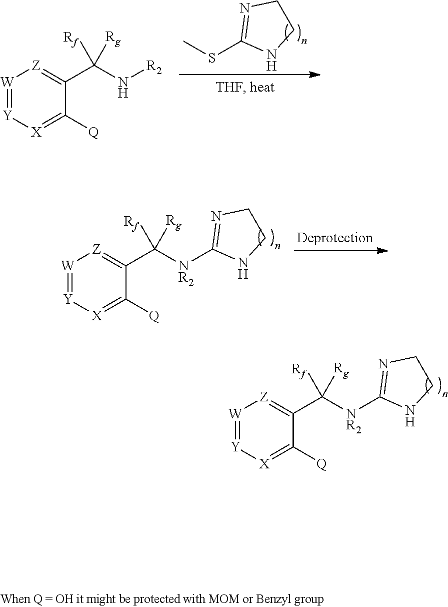

### Step 1

The appropriate amine (1.1 equiv.) was added to a solution of the appropriate cyclic methylisothiourea (1 equiv.) in dry THF (1M) and the reaction was stirred at 40° C. until completion. Water was added and the resulting mixture was extracted with DCM (×3). The combined organic layers were dried Na₂SO₄, filtered and concentrated under vacuum. The crude product was then purified by prep HPLC.

Step 2—See deprotection steps in General Procedure A of acylguanidine synthesis.

### General Procedure B

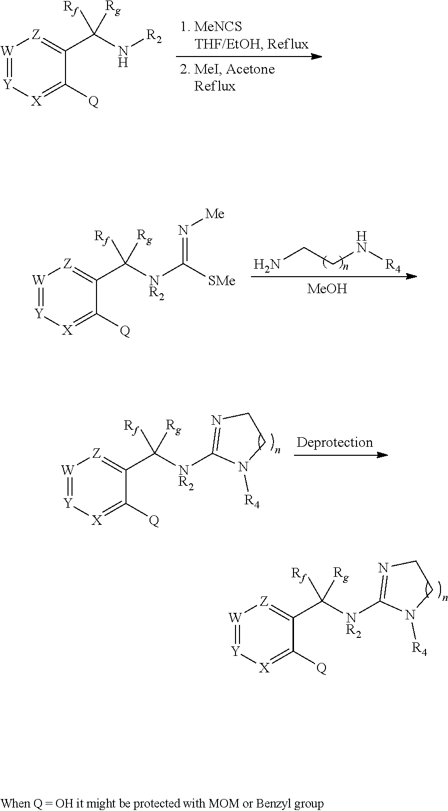

### Step 1

The appropriate amine (1 equiv.) was dissolved in a mixture of THF/EtOH (0.2M) then MeNCS (5 equiv.) was added and reaction refluxed until completion. Water was added and the resulting mixture was extracted with DCM (×3). The combined organic layers were dried Na₂SO₄, filtered and concentrated under vacuum. The crude product was used for next step without any further purification.

### Step 2

The above intermediate (1 equiv.) was dissolved in Acetone (0.1M) and MeI (5 equiv.) was added and reaction refluxed until completion. Water was added and the resulting mixture was extracted with DCM (×3). The combined organic layers were dried over Na₂SO₄, filtered and concentrated under vacuum. The crude product was then purified by flash chromatography.

### Step 3

The above intermediate (1 equiv.) was dissolved in MeOH (0.1M) and the appropriate diamine (2 equiv.) was added and reaction stirred at RT until completion. Solvent evaporated and crude was purified by prep HPLC.

Step 4—See deprotection steps in General Procedure A of acylguanidine synthesis.

### General Procedure C

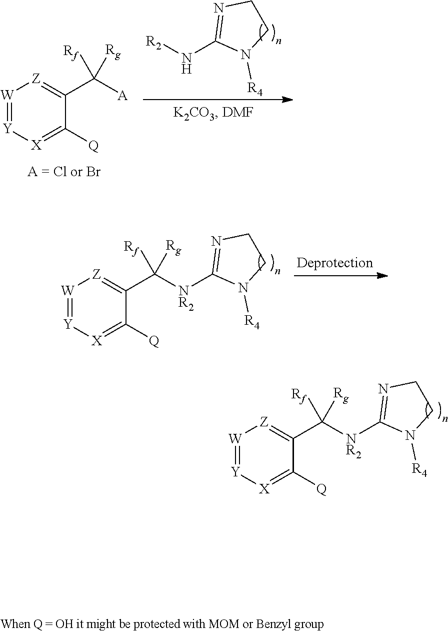

### Step 1

To a solution of the appropriate cyclic guanidine (1 equiv.) in DMF (0.2M) potassium carbonate (1.5 equiv.) was added followed by the appropriate halide (1.1 eq). The reaction was stirred at RT until completion. Water was added and the resulting mixture was extracted with DCM (×3). The combined organic layers were dried over Na₂SO₄, filtered and concentrated under vacuum. The crude product was then purified by prep HPLC.

Step 2—See deprotection steps in General Procedure A of acylguanidine synthesis.

### General Procedure D

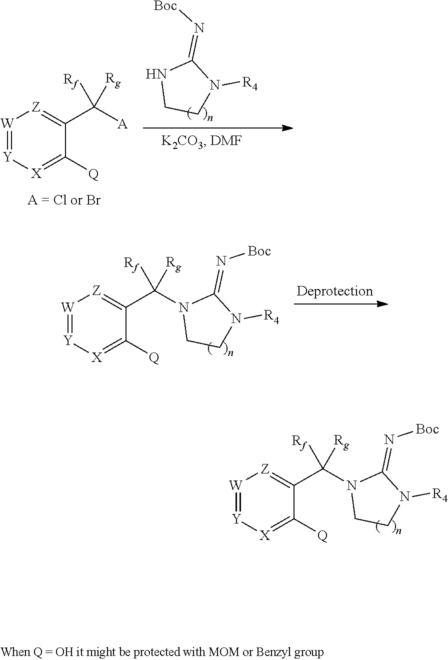

### Step 1

To a solution of the appropriate cyclic guanidine (1 equiv.) in DMF (0.2M) potassium carbonate (1.5 equiv.) was added followed by the appropriate halide (1.1 eq). The reaction was stirred at RT until completion. Water was added and the resulting mixture was extracted with DCM (×3). The combined organic layers were dried over Na₂SO₄, filtered and concentrated under vacuum. The crude product was then purified by prep HPLC.

Step 2—See deprotection steps in General Procedure A of acylguanidine synthesis.

### General Procedure E

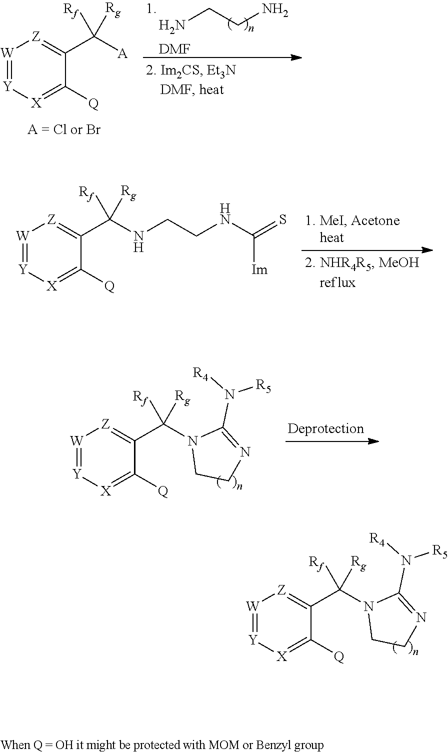

### Step 1

To a solution of the appropriate halide (1 equiv.) in DMF (0.2M) the appropriate diamine (5 equiv.) was added and reaction stirred at RT until completion. Water was added and the resulting mixture was extracted with DCM (×3). The combined organic layers were dried over Na₂SO₄, filtered and concentrated under vacuum. The crude product was used for next step without any further purification.

### Step 2

The above intermediate (1 equiv.) was dissolved in DMF (0.2M) then triethylamine (5 equiv.) was added followed by Im₂CS (1 eq). Reaction was stirred at 70° C. until completion. Water was added and the resulting mixture was extracted with DCM (×3). The combined organic layers were dried over Na₂SO₄, filtered and concentrated under vacuum. The crude product was then purified by flash chromatography.

### Step 3

The above intermediate (1 equiv.) was dissolved in Acetone (0.1M) and MeI (5 equiv.) was added and reaction refluxed until completion. Water was added and the resulting mixture was extracted with DCM (×3). The combined organic layers were dried over Na₂SO₄, filtered and concentrated under vacuum. The crude product was used for next step without any further purification.

### Step 4

The above intermediate (1 equiv.) was dissolved in MeOH (0.1M) and the appropriate amine (5 equiv.) was added and reaction refluxed until completion. Solvent was evaporated and the crude product was purified by prep HPLC.

Step 5—See deprotection steps in General Procedure A of acylguanidine synthesis.

### General Procedure F

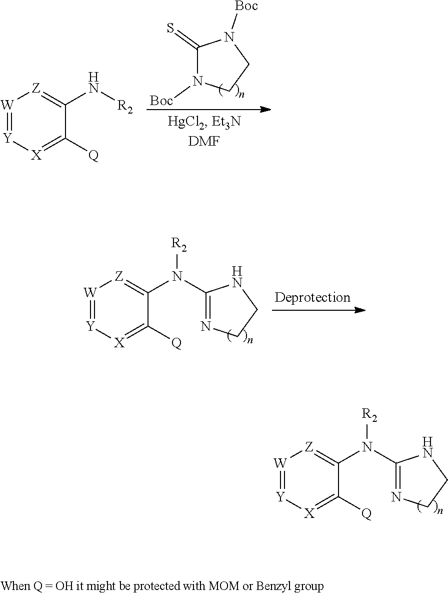

### Step 1

To a solution of the appropriate amine (1 equiv.) in DMF (0.2M) the appropriate cyclic thiourea (1.2 equiv.), triethylamine (5 equiv.) and HgCl₂ (1 equiv.) were added and the reaction was stirred at RT until completion. The reaction mixture was then diluted with EtOAc and filtered through Celite. The filtrate was washed with saturated NH₄Cl and brine. The organic solution was then dried over Na₂SO₄, filtered and concentrated under vacuum. The crude product was then purified by prep HPLC.

Step 2—See deprotection steps in General Procedure A of acylguanidine synthesis.

### General Procedure G

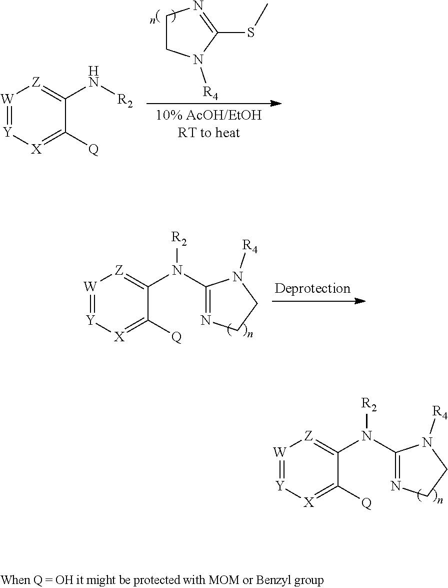

### Step 1

The appropriate amine (1 equiv.) was dissolved in 10% AcOH/EtOH (0.2M) and the appropriate cyclic thiomethyl reagent (1.5 equiv.) was added at RT. Reaction then was heated and stirred until completion. Solvent was removed under vacuum and the crude product was purified by prep HPLC.

Step 2—See deprotection steps in General Procedure A of acylguanidine synthesis.

### General Procedure H

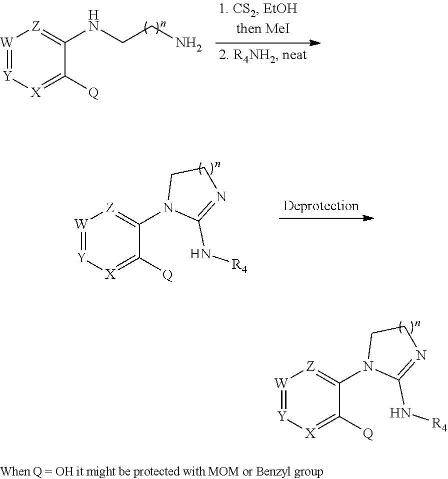

### Step 1

A solution of the appropriate diamine derivative (1 equiv.) in ethanol (0.5M) was stirred while carbon disulfide (1 equiv.) was added dropwise, and the mixture was heated at 50° C. for 3 h. The solution was then cooled, and the precipitated intermediate was collected. MeI (2.5 equiv.) was added dropwise to a suspension of the above solid (1 equiv.) in methanol (0.5M) and the reaction mixture heated at 60° C. until starting material disappeared. The reaction mixture was concentrated under vacuum to an oil, and then excess of the appropriate amine (10 equiv.) was added. The mixture was then heated at 90° C. until completion. 0.1N aqueous NaOH was added and the resulting solution was extracted with EtOAc (×3). The combined organic layers were washed with brine, dried over Na₂SO₄, filtered and concentrated under vacuum. The crude mixture was purified by prep HPLC.

Step 2—See deprotection steps in General Procedure A of acylguanidine synthesis.

### General Procedure I

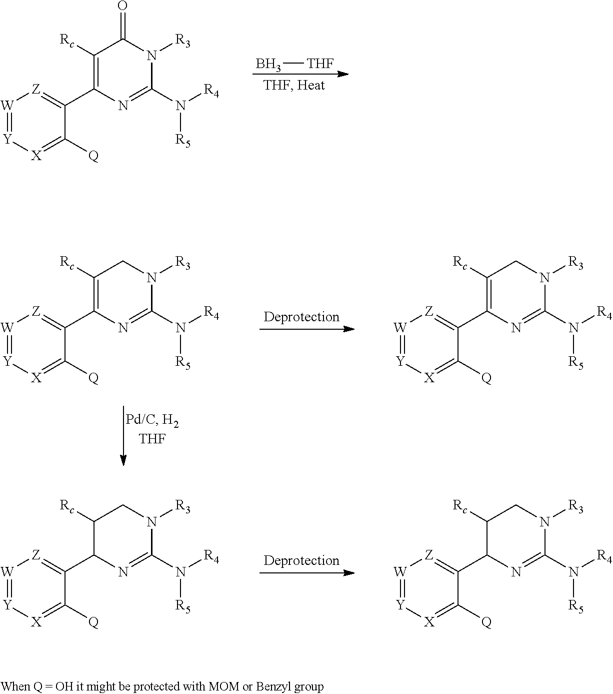

### Step 1

To a solution of the appropriate starting material (1 equiv.) in THF (0.2M) BH₃-THF (2M in THF, 3 equiv.) was added and reaction refluxed until full conversion. Reaction was cooled down to RT and 1M HCl was added and the reaction mixture was stirred at RT until the two layers are well separated. The mixture was then extracted with EtOAc (×3) and the combined organic layers were dried over Na₂SO₄, filtered and concentrated under vacuum. The crude was purified by prep HPLC.

### Step 2

The above intermediate (1 equiv.) was dissolved in THF (0.1M) and 10% Palladium on carbon (0.1 eqs) was added and reaction stirred under hydrogen atmosphere until completion. The reaction mixture was filtered through a pad of Celite and solvent was removed under vacuum. The crude product was then purified by prep HPLC.

Step 3—See deprotection steps in General Procedure A of acylguanidine synthesis.

### General Procedure K

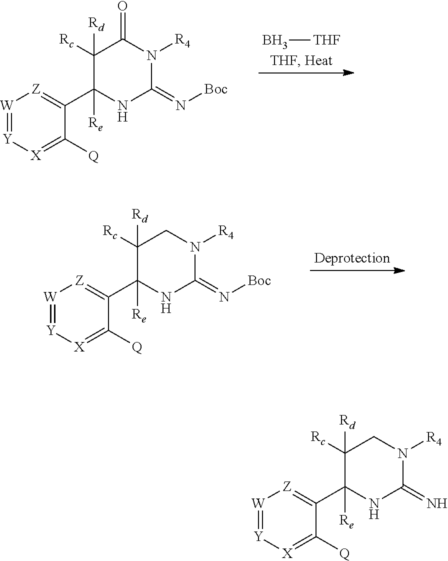

### Step 1

To a solution of the appropriate starting material (1 equiv.) in THF (0.2M) BH₃-THF (2M in THF, 3 equiv.) was added and reaction refluxed until full conversion. Reaction was cooled down to RT and 1M HCl was added and the reaction mixture was stirred at RT until the two layers are well separated. The mixture was then extracted with EtOAc (×3) and the combined organic layers were dried over Na₂SO₄, filtered and concentrated under vacuum. The crude was purified by prep HPLC.

Step 2—See deprotection steps in General Procedure A of acylguanidine synthesis.

### Synthesis of Aminoimidazoles and Aminoimidazolines

### General Procedure A

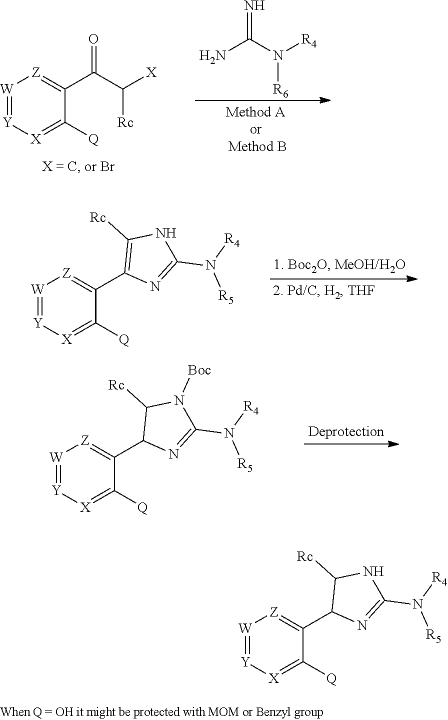

### Step 1—Method A

To a solution of the appropriate halide derivative (1 equiv.) in DMF (0.2M) was added the appropriate guanidine (2 equiv.) and reaction was stirred at RT until completion. The solvent was removed under vacuum and the imidazole product was purified by prep HPLC.

### Step 1—Method B

A mixture of the corresponding halide derivative (1 equiv.) and guanidine (3 equiv.) in anhydrous acetonitrile (0.1M) was heated at 100° C. using microwave irradiation for 15 min. The solvent was removed and the crude product was purified by prep HPLC.

### Step 2

To a suspension of the appropriate imidazole derivative (1 equiv.) in a mixture of methanol/water 2:1 (0.15M) Boc-anhydride (1.1 equiv.) was added and the mixture was stirred at RT until completion. The precipitate was filtered off, washed with methanol and the crude product was used for next reaction without any further purification.

### Step 3

The above intermediate (1 equiv.) was dissolved in THF (0.1M) and 10% Palladium on carbon (0.1 equiv.) was added and reaction stirred under hydrogen atmosphere until completion. The reaction mixture was filtered through a pad of Celite and solvent was removed under vacuum. The crude product was then purified by prep HPLC.

Step 4—See deprotection steps in General Procedure A of acylguanidine synthesis.

### General Procedure B

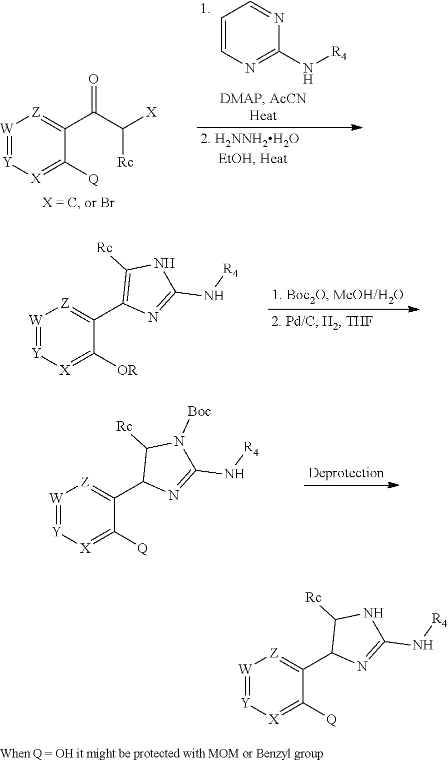

### Step 1

To a solution of the appropriate pyrimidine (1 equiv.) and halide derivative (1.2 equiv.) in acetonitrile (0.2M) DMAP (0.01 equiv.) was added. After being stirred at 85° C. until completion, the reaction mixture was filtered, washed with acetonitrile and ether and dried to give the pyrimidiniun salt intermediate.

### Step 2

To a suspension of the above intermediate (1 equiv.) in ethanol (0.2M) hydrazine hydrate (35% hydrazine in solution, 7 equiv.) was added, and the reaction was placed in a microwave reactor and heated at 120° C. for 40 min. The mixture was cooled to RT, the solvent evaporated and the resulting residue was purified by prep HPLC.

### Step 3

To a suspension of the appropriate imidazole derivative (1 equiv.) in a mixture of methanol/water 2:1 (0.15M) Boc-anhydride (1.1 eqs) was added and the mixture was stirred at RT until completion. The precipitate was filtered off, washed with methanol and the crude product was used for next reaction without any further purification.

### Step 4

The above intermediate (1 equiv.) was dissolved in THF (0.1M) and 10% Palladium on carbon (0.1 eqs) was added and reaction stirred under hydrogen atmosphere until completion. The reaction mixture was filtered through a pad of Celite and solvent was removed under vacuum. The crude product was then purified by prep HPLC.

Step 5—See deprotection steps in General Procedure A of acylguanidine synthesis.

### General Procedure C

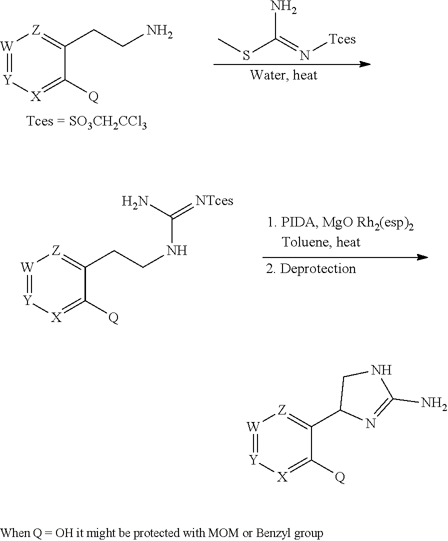

### Step 1

A flask was charged with S-methyl-N-(2,2,2 trichloroethoxysulfonyl)isothiourea (1 equiv.), the appropriate amine (1 equiv.), and H₂O (1M). The reaction was stirred at 100° C. until completion. The reaction mixture was cooled to RT and DCM was added. The biphasic solution was extracted with DCM (×3) and the combined organic layers were dried over Na₂SO₄, filtered and concentrated under reduced pressure. Purification of the isolated material by column chromatography afforded the desired guanidine substrate.

### Step 2

A flame-dried flask was charged with Tces guanidine (1 equiv.), Rh₂(esp)₂ (0.02 equiv.), PhI(OAc)₂ (1.65 equiv.), and MgO (2.5 eq). The reaction mixture was placed briefly under vacuum, and the flask then backfilled with nitrogen. This process was repeated two additional times prior to the addition of deoxygenated toluene (0.1M). The resulting suspension was heated to 40° C. and stirred until completion. The reaction mixture was then cooled to RT, and the crude was purified by prep HPLC.

Step 3—See deprotection steps in General Procedure A of acylguanidine synthesis.

### General Procedure D

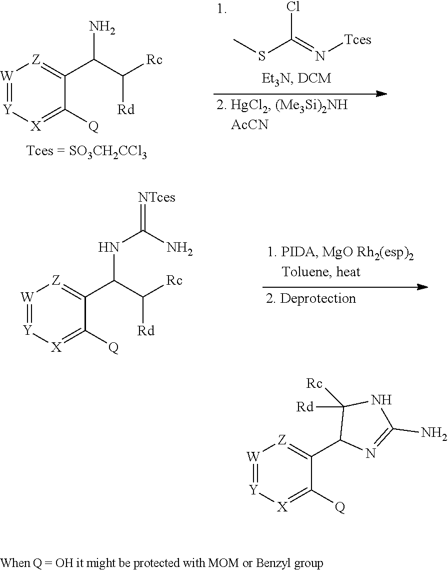

### Step 1

A solution of (2,2,2-trichloroethoxysulfonyl)carbonchloroimidothioic acid methyl ester (1.1 equiv.) in DCM (0.2M) was cooled to 0° C. and the appropriate amine (1 equiv.) was added dropwise. To the resulting mixture was added triethylamine (1.2 eq). The reaction was warmed to RT and stirred for 4 h. The solution was then concentrated under vacuum. Purification by chromatography on silica gel furnished the isothiourea product.

### Step 2

To a solution of isothiourea (1 equiv.) in MeCN (0.1M) was added successively (Me₃Si)₂NH (2.5 equiv.) and HgCl₂ (1.1 eq). After 1 h the milky white suspension was filtered through Celite and the filtrate was concentrated under reduced pressure. Purification of the crude residue by column chromatography gave the desired N-Tces guanidine.

### Step 3

A flame-dried flask was charged with Tces guanidine (1 equiv.), Rh₂(esp)₂ (0.02 equiv.), PhI(OAc)₂ (1.65 equiv.), and MgO (2.5 eq). The reaction mixture was placed briefly under vacuum, and the flask then backfilled with nitrogen. This process was repeated two additional times prior to the addition of deoxygenated toluene (0.1M). The resulting suspension was heated to 40° C. and stirred until completion. The reaction mixture was then cooled to RT, and the crude was purified by prep HPLC.

Step 4—See deprotection steps in General Procedure A of acylguanidine synthesis.

### Synthesis of Aminopyrimidinone

### General Procedure A

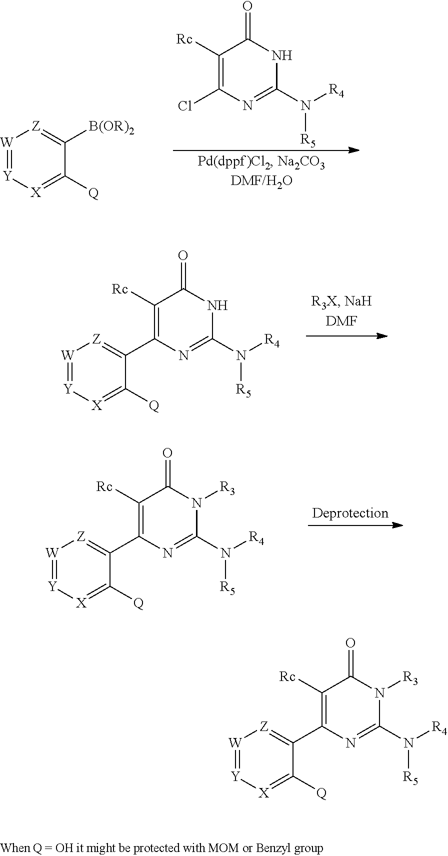

### Step 1

To a solution of the appropriate boronic acid (1 equiv.) in a mixture of DMF/Water 3:1 (0.1M), sodium carbonate (1.5 equiv.) was added followed by Pd(dppf)Cl₂ (0.1 equiv.) and reaction stirred at RT until completion. Water was added and the resulting mixture was extracted with DCM (×3). The combined organic layers were dried over Na₂SO₄, filtered and concentrated under vacuum. The crude product was then purified by prep HPLC.

### Step 2

The above intermediate (1 equiv.) was dissolved in DMF (0.1 equiv.), then at 0° C. NaH (1.2 equiv.) was added and reaction stirred at RT for 30 min before the corresponding electrophile (1.5 equiv.) was added. Reaction was stirred at RT until completion. Water was added and the resulting mixture was extracted with DCM (×3). The combined organic layers were dried over Na₂SO₄, filtered and concentrated under vacuum. The crude product was then purified by prep HPLC.

Step 3—See deprotection steps in General Procedure A of acylguanidine synthesis.

### General Procedure B

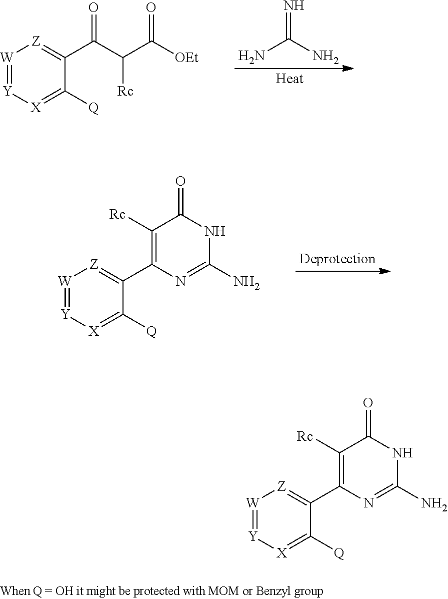

### Step 1

To a suspension of guanidine carbonate (1.5-5 equiv.) in ethanol (2 mL/mmol) was added the appropriate β-ketoester (1 equiv.), and the reaction mixture heated at 80° C. for 15-64 h. Following reaction completion by TLC, the mixture was cooled to RT, filtered and the crude product was purified by prep HPLC.

Step 2—See deprotection steps in General Procedure A of acylguanidine synthesis.

### Synthesis of Aminopyrimidinethione

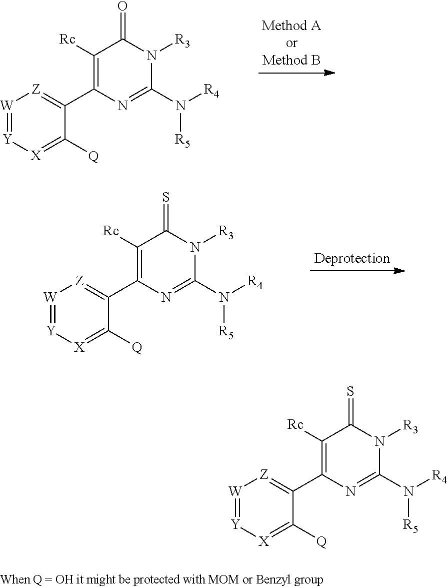

### Step 1—Method A

The appropriate starting material (1 equiv.) was dissolved in Toluene (0.05M), then Lawesson's reagent was added (1 equiv.) and reaction refluxed until completion. Solvent was then removed under vacuum and crude was purified by prep HPLC.

### Step 1—Method B

The appropriate starting material (1 equiv.) was dissolved in pyridine and phosphorus pentasulfide (1 equiv.) was added and reaction refluxed until completion. The reaction was then cooled down and poored into ice water. The resulting mixture was then extracted with Ethyl Acetate and the combined organic layers were dried over Na₂SO₄, filtered and concentrated under vacuum. The crude was then purified by prep HPLC

Step 2—See deprotection steps in General Procedure A of acylguanidine synthesis.

### Synthesis of Aminothiadiazine Dioxide

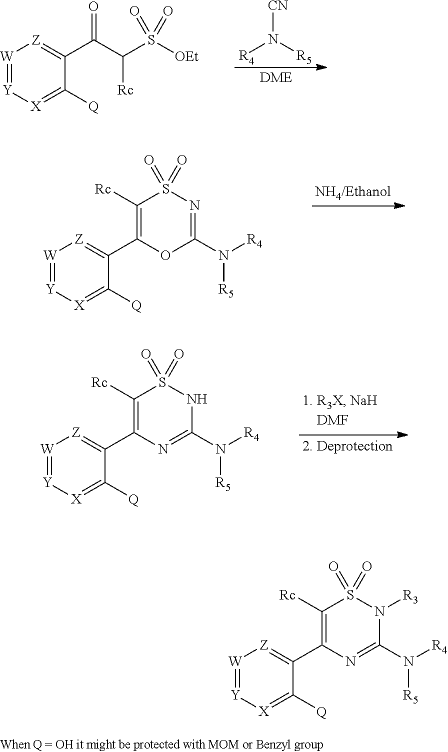

### Step 1

The appropriate sulfonyl chloride (1 equiv.) was dissolved in DME (0.1M) then the appropriate cyanamide (1.2 equiv.) was added and reaction stirred at 80° C. until completion. Solvent evaporated under reduced pressure and the crude product was purified by prep HPLC or flash chromatography.

### Step 2

The above intermediate (1 equiv.) was then dissolved in NH₄ in Ethanol and reaction stirred at RT until completion. Solvent evaporated under reduced pressure and the crude product was purified by prep HPLC.

### Step 3

The above product (1 equiv.) was dissolved in DMF (0.1 equiv.), then at 0° C. NaH (1.2 equiv.) was added and reaction stirred at RT for 30 min before the corresponding electrophile (1.5 equiv.) was added. Reaction was stirred at RT until completion. Water was added and the resulting mixture was extracted with DCM (×3). The combined organic layers were dried over Na₂SO₄, filtered and concentrated under vacuum. The crude product was then purified by prep HPLC.

Step 4—See deprotection steps in General Procedure A of acylguanidine synthesis.

### Synthesis of Iminotetrahydropyrimidinone

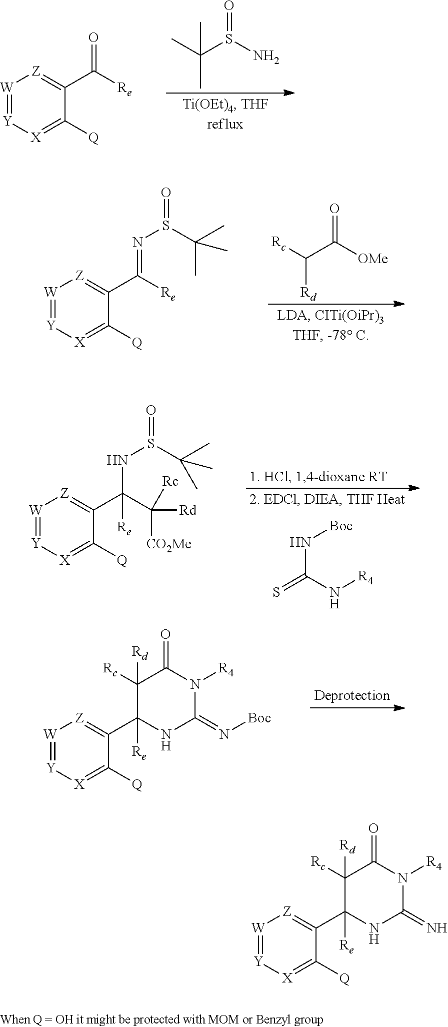

### Step 1

To a pressure vessel containing a solution of the appropriate ketone (1 equiv.) in THF (0.8M) was added the appropriate enantiomer of 2-methylpropane-2-sulfinamide (2.3 equiv.) followed by titanium(IV)ethoxide (0.45 eq). The vessel was sealed and the mixture was heated to 75° C. for several hours until completion. After that time, the mixture was cooled to room temperature, poured into water and then the mixture was filtered. The filter cake was washed with DCM and the filtrate was extracted with DCM. The organic layer was dried over Na₂SO₄, filtered, and concentrated under vacuum. The crude residue was purified by flash chromatography.

### Step 2

A solution of n-butyllithium (2.5 M in hexanes, 2.5 equiv.) was added slowly at 0° C. to a solution of DIEA (2.5 equiv.) in THF (0.9M) and the resulting solution was stirred at 0° C. for 0.5 h. The reaction mixture was then cooled to −78° C. followed by dropwise addition of a solution of the appropriate methyl acetate (2 equiv.) in THF (3.5M). The resulting reaction mixture was stirred at −78° C. for 1.5 h. After that time, a solution of chlorotitanium triisopropoxide (3 equiv.) in THF (2M) was added slowly and the reaction was stirred for 2 hours. A solution of the above intermediate (1 equiv.) in THF (12M) was then added slowly and the reaction stirred until completion. The reaction was quenched at −78° C. by gradual addition of water and the resulting mixture allowed to warm to RT overnight. The resultant slurry was diluted with EtOAc, filtered, and the filter cake was rinsed with EtOAc. The filtrate layers were separated and the organic layer was washed with water, dried over Na₂SO₄, filtered and concentrated under vacuum. The residue was purified by flash chromatography.

### Step 3

A solution of HCl (4.0 M in 1,4-dioxane, 8 equiv.) was added to a solution of the above intermediate (1 equiv.) in 7:1 methylene chloride/methanol (0.3M) and the reaction mixture stirred at RT until completion. The reaction mixture was then concentrated under vacuum and the residue dried under high vacuum to afford the crude intermediate amine-HCl salt.

### Step 4

To a portion of the amine (1 equiv.) in DMF (1M) was added N-(dimethylamino)propyl)-3-ethylcarbodiimide hydrochloride (1.5 equiv.) and DIEA (4.5 eq). The reaction mixture stirred at 45° C. until completion. After that time, the reaction mixture was diluted with water and EtOAc and the resultant mixture was stirred vigorously until the phases cleared. The layers were separated and the aqueous layer extracted with EtOAc (×2). The combined organic layers were sequentially washed with 1 N HCl, sat. Na₂CO₃, and brine, then dried over Na₂SO₄, filtered and concentrated under vacuum. The residue was purified by flash chromatography or prep HPLC.

Step 5—See deprotection steps in General Procedure A of acylguanidine synthesis.

### Synthesis of Iminotetrahydropyrimidinethione

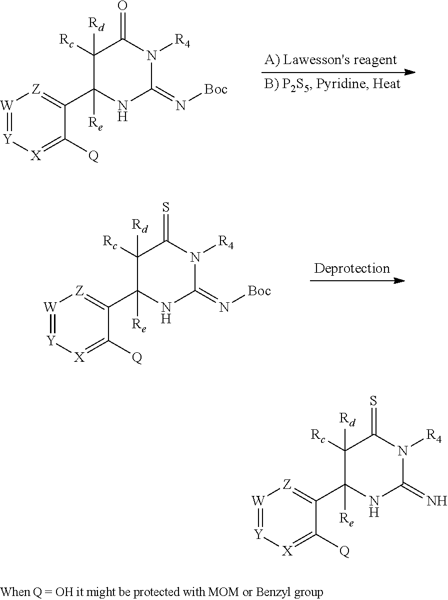

### Step 1—Method A

The appropriate starting material (1 equiv.) was dissolved in Toluene (0.05M), then Lawesson's reagent was added (1 equiv.) and reaction refluxed until completion. Solvent was then removed under vacuum and crude was purified by prep HPLC.

### Step 1—Method B

The appropriate starting material (1 equiv.) was dissolved in pyridine and phosphorus pentasulfide (1 equiv.) was added and reaction refluxed until completion. The reaction was then cooled down and poored into ice water. The resulting mixture was then extracted with Ethyl Acetate and the combined organic layers were dried over Na₂SO₄, filtered and concentrated under vacuum. The crude was then purified by prep HPLC

### Synthesis of Iminothiadiazinane Dioxide

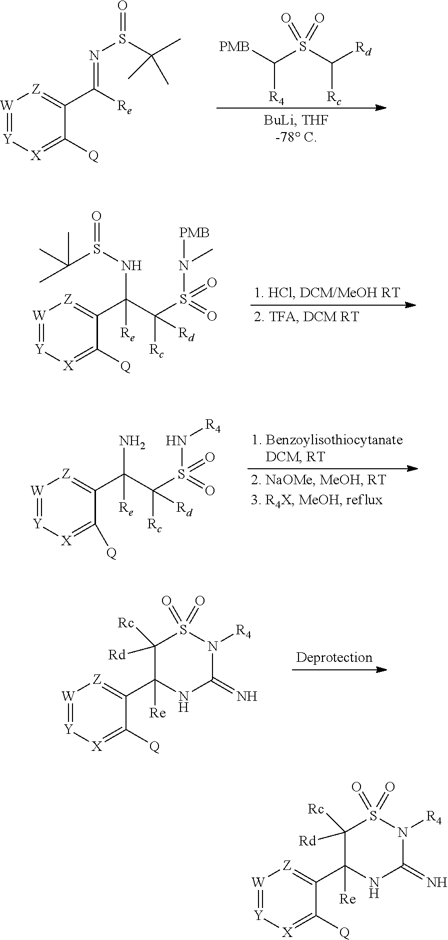

### Step 1

To a flame dried round bottom flask containing a solution of the appropriate PMB protected solfonamide (2.5 equiv.) in THF (0.3M) at −45° C. under an atmosphere of nitrogen was added dropwise a solution of n-BuLi (2.5 equiv.) and the mixture was stirred for 20 min. After that time, the solution was cooled to −78° C. and transferred by cannula into a precooled solution (−78° C.) of the appropriate sulfinamide intermediate (1 equiv.) in THF (0.1M). The resultant mixture was stirred at −78° C. until completion. The mixture was then quenched with water and the mixture was slowly warmed to RT. The mixture was then extracted with DCM (×3). The combined organic layers were dried over Na₂SO₄, filtered, and concentrated under vacuum. The crude residue was purified by flash chromatography.

### Step 2

To a solution of the above intermediate (1 equiv.) in a mixture of DCM:MeOH (3:1, 0.05M) at RT was added a solution of HCl (4 N in dioxane, 6 eq). The solution was stirred at RT until completion and then the solution was concentrated under vacuum. The resultant residue was re-concentrated under vacuum from toluene (×3) and the mixture was dried further under high vacuum.

### Step 3

To the residue was added DCM (0.5M) followed by TFA (same volume of DCM) and 1,3-dimethoxybenzene (half volume of DCM). The reaction mixture was stirred at RT until completion. After that time, the volatiles were removed under reduced pressure. To the resultant mixture was added 2 M HCl (aq.) and the mixture was extracted with Et₂O (×3). The aqueous layer was then adjusted to ˜ pH 10 with the addition of solid Na₂CO₃. The aqueous layer was then extracted with DCM (×3). The combined organic layers were dried over Na₂SO₄, filtered, and concentrated under vacuum. The crude was used for next step without any further purification.

### Steps 4-6

To a solution of the above intermediate (1 equiv.) in DCM (0.15M) was added benzoylisothiocyanate (1.2 equiv.) and the mixture was stirred at RT overnight and the solvent was removed under vacuum. To the residue was added MeOH (0.2M) followed by the addition of a solution of sodium methoxide (25% in MeOH, 2.5 eq). The mixture was stirred at RT for 1 hour after which the solvent was removed in vacuo. The residue was partitioned between EtOAc and water. The aqueous layer was then adjusted to ˜pH 7 with solid NH₄Cl and the mixture was extracted with EtOAc (×3). The combined organic layers were dried over Na₂SO₄, filtered, and concentrated in vacuo to afford the crude thiourea.

To this residue was added EtOH (0.15M) followed by the appropriate electrophile (1.2 equiv.) and the mixture was heated to 70° C. until completion. The mixture was then cooled to RT and the solvent was removed under vacuum. The residue was partitioned between water and DCM and the aqueous layer was basified (pH˜10) with sat. Na₂CO₃ (aq.). The layers were separated and the aqueous layer was extracted with DCM (×2). The combined organic layers were dried over Na₂SO₄, filtered, and concentrated under vacuum. The crude residue was purified by flash chromatography or prep HPLC.

Step 7—See deprotection steps in General Procedure A of acylguanidine synthesis.

### Synthesis of Acylguanidine Dimers

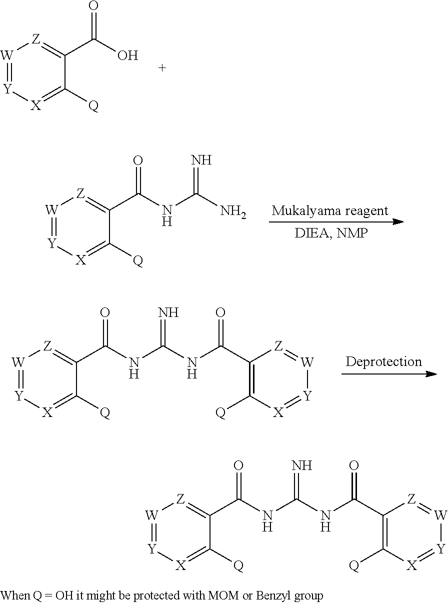

### Step 1

To a solution of the appropriate carboxylic acid (1 equiv.) in NMP (0.2M), DIEA (2 equiv.) and Mukaiyama reagent (1.5 equiv.) were added and reaction stirred at RT for 10-15 min before the appropriate acylguanidine (1.2 equiv.) was added. Reaction stirred at RT until full conversion. Water was added and the mixture was extracted with DCM (×3). The combined organic layers were dried over Na₂SO₄, filtered and concentrated under vacuum. The crude product was purified by prep HPLC.

Step 2—See deprotection steps in General Procedure A of acylguanidine synthesis.

### Synthesis of Aminooxadiazoles

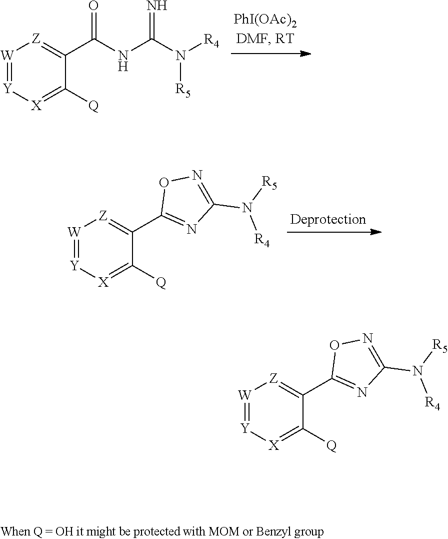

### Step 1

To a solution of the appropriate acylguanidine (1 eq) in DMF (0.2M), PhI(OAc)₂ (1.5 eq) was added and reaction was stirred at RT until full conversion. Water was added and mixture extracted with EtOAc (×3). The combined organic layers were dried over Na₂SO₄, filtered and concentrated under vacuum. The crude product was purified by prep HPLC.

Step 2—See deprotection steps in General Procedure A of acylguanidine synthesis.

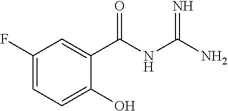 [SMILES: N=C(N)NC(=O)c1cc(F)ccc1O]

This compound was prepared using General Procedure A of acylguanidine synthesis, Method A (LC/MS m/z 198.17 [M+H⁺])

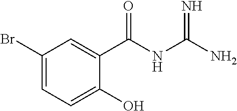 [SMILES: N=C(N)NC(=O)c1cc(Br)ccc1O]

This compound was prepared using General Procedure A of acylguanidine synthesis, Method A (LC/MS m/z 258.09 [M+H⁺])

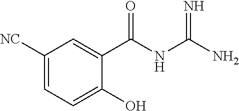 [SMILES: N#Cc1ccc(O)c(C(=O)NC(=N)N)c1]

This compound was prepared using General Procedure A of acylguanidine synthesis, Method A (LC/MS m/z 205.14 [M+H⁺])

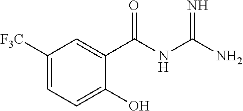 [SMILES: N=C(N)NC(=O)c1cc(C(F)(F)F)ccc1O]

This compound was prepared using General Procedure A of acylguanidine synthesis, Method A (LC/MS m/z 248.16 [M+H⁺])

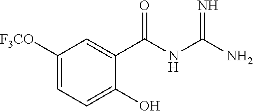 [SMILES: N=C(N)NC(=O)c1cc(OC(F)(F)F)ccc1O]

This compound was prepared using General Procedure A of acylguanidine synthesis, Method A (LC/MS m/z 264.14 [M+H⁺])

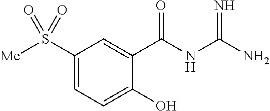 [SMILES: CS(=O)(=O)c1ccc(O)c(C(=O)NC(=N)N)c1]

This compound was prepared using General Procedure A of acylguanidine synthesis, Method A (LC/MS m/z 258.15 [M+H⁺])

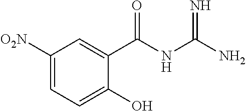 [SMILES: N=C(N)NC(=O)c1cc([N+](=O)[O-])ccc1O]

This compound was prepared using General Procedure A of acylguanidine synthesis, Method A (LC/MS m/z 225.17 [M+H⁺])

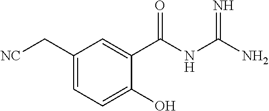 [SMILES: N#CCc1ccc(O)c(C(=O)NC(=N)N)c1]

This compound was prepared using General Procedure A of acylguanidine synthesis, Method A (LC/MS m/z 219.25 [M+H⁺])

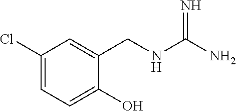 [SMILES: N=C(N)NCc1cc(Cl)ccc1O]

This compound was prepared using General Procedure A of guanidine synthesis (LC/MS m/z 200.18 [M+H⁺])

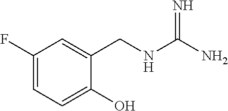 [SMILES: N=C(N)NCc1cc(F)ccc1O]

This compound was prepared using General Procedure A of guanidine synthesis (LC/MS m/z 184.19 [M+H⁺])

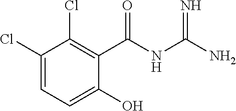 [SMILES: N=C(N)NC(=O)c1c(O)ccc(Cl)c1Cl]

This compound was prepared using General Procedure A of acylguanidine synthesis, Method A (LC/MS m/z 248.16 [M+H⁺])

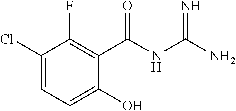 [SMILES: N=C(N)NC(=O)c1c(O)ccc(Cl)c1F]

This compound was prepared using General Procedure A of acylguanidine synthesis, Method A (LC/MS m/z 232.14 [M+H⁺])

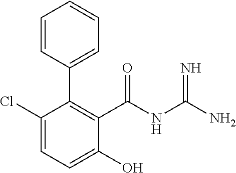 [SMILES: N=C(N)NC(=O)c1c(O)ccc(Cl)c1-c1ccccc1]

This compound was prepared using General Procedure A of acylguanidine synthesis, Method B (LC/MS m/z 290.29 [M+H⁺])

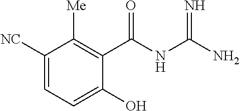 [SMILES: Cc1c(C#N)ccc(O)c1C(=O)NC(=N)N]

This compound was prepared using General Procedure A of acylguanidine synthesis, Method A (LC/MS m/z 219.25 [M+H⁺])

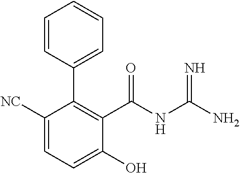 [SMILES: N#Cc1ccc(O)c(C(=O)NC(=N)N)c1-c1ccccc1]

This compound was prepared using General Procedure A of acylguanidine synthesis 1, Method B (LC/MS m/z 281.31 [M+H⁺])

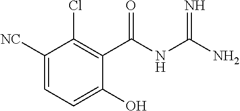 [SMILES: N#Cc1ccc(O)c(C(=O)NC(=N)N)c1Cl]

This compound was prepared using General Procedure A of acylguanidine synthesis, Method B (LC/MS m/z 239.30 [M+H⁺])

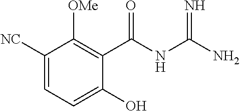 [SMILES: COc1c(C#N)ccc(O)c1C(=O)NC(=N)N]

This compound was prepared using General Procedure A of acylguanidine synthesis, Method B (LC/MS m/z 235.24 [M+H⁺])

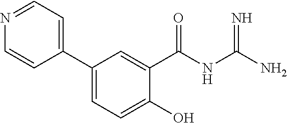 [SMILES: N=C(N)NC(=O)c1cc(-c2ccncc2)ccc1O]

This compound was prepared using General Procedure A of acylguanidine synthesis, Method A (LC/MS m/z 257.24 [M+H⁺])

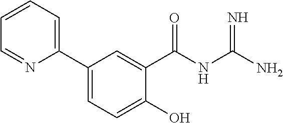 [SMILES: N=C(N)NC(=O)c1cc(-c2ccccn2)ccc1O]

This compound was prepared using General Procedure A of acylguanidine synthesis, Method A (LC/MS m/z 257.27 [M+H⁺])

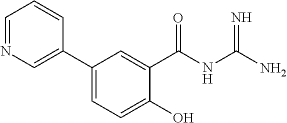 [SMILES: N=C(N)NC(=O)c1cc(-c2cccnc2)ccc1O]

This compound was prepared using General Procedure A of acylguanidine synthesis, Method A (LC/MS m/z 257.24 [M+H⁺])

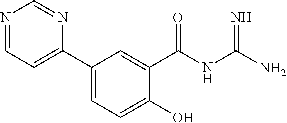 [SMILES: N=C(N)NC(=O)c1cc(-c2ccncn2)ccc1O]

This compound was prepared using General Procedure A of acylguanidine synthesis, Method A (LC/MS m/z 258.32 [M+H⁺])

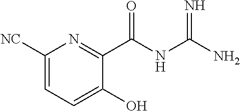 [SMILES: N#Cc1ccc(O)c(C(=O)NC(=N)N)n1]

This compound was prepared using General Procedure A of acylguanidine synthesis, Method A (LC/MS m/z 206.20 [M+H⁺])

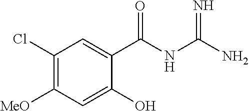 [SMILES: COc1cc(O)c(C(=O)NC(=N)N)cc1Cl]

This compound was prepared using General Procedure A of acylguanidine synthesis, Method A (LC/MS m/z 244.14 [M+H⁺])

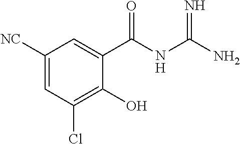 [SMILES: N#Cc1cc(Cl)c(O)c(C(=O)NC(=N)N)c1]

This compound was prepared using General Procedure A of acylguanidine synthesis, Method A (LC/MS m/z 239.18 [M+H⁺])

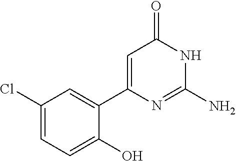 [SMILES: Nc1nc(-c2cc(Cl)ccc2O)cc(=O)[nH]1]

This compound was prepared using General Procedure A of aminopyrimidinone synthesis (LC/MS m/z 238.09 [M+H⁺])

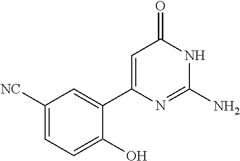 [SMILES: N#Cc1ccc(O)c(-c2cc(=O)[nH]c(N)n2)c1]

This compound was prepared using General Procedure A of aminopyrimidinone synthesis (LC/MS m/z 229.22 [M+H⁺])

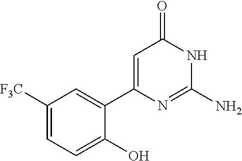 [SMILES: Nc1nc(-c2cc(C(F)(F)F)ccc2O)cc(=O)[nH]1]

This compound was prepared using General Procedure A of aminopyrimidinone synthesis (LC/MS m/z 272.23 [M+H⁺])

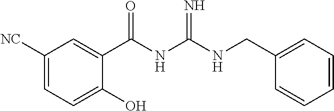 [SMILES: N#Cc1ccc(O)c(C(=O)NC(=N)NCc2ccccc2)c1]

This compound was prepared using General Procedure B of acylguanidine synthesis (LC/MS m/z 295.32 [M+H⁺])

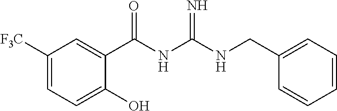 [SMILES: N=C(NCc1ccccc1)NC(=O)c1cc(C(F)(F)F)ccc1O]

This compound was prepared using General Procedure C of acylguanidine synthesis (LC/MS m/z 338.34 [M+H⁺])

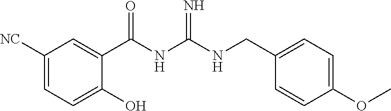 [SMILES: COc1ccc(CNC(=N)NC(=O)c2cc(C#N)ccc2O)cc1]

This compound was prepared using General Procedure B of acylguanidine synthesis (LC/MS m/z 325.35 [M+H⁺])

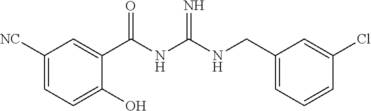 [SMILES: N#Cc1ccc(O)c(C(=O)NC(=N)NCc2cccc(Cl)c2)c1]

This compound was prepared using General Procedure B of acylguanidine synthesis (LC/MS m/z 329.33 [M+H⁺])

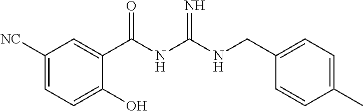 [SMILES: Cc1ccc(CNC(=N)NC(=O)c2cc(C#N)ccc2O)cc1]

This compound was prepared using General Procedure B of acylguanidine synthesis (LC/MS m/z 309.40 [M+H⁺])

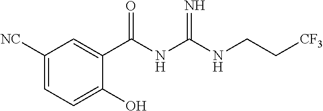 [SMILES: N#Cc1ccc(O)c(C(=O)NC(=N)NCCC(F)(F)F)c1]

This compound was prepared using General Procedure B of acylguanidine synthesis (LC/MS m/z 301.31 [M+H⁺])

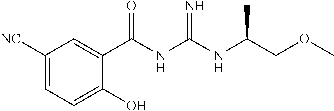 [SMILES: COC[C@H](C)NC(=N)NC(=O)c1cc(C#N)ccc1O]

This compound was prepared using General Procedure B of acylguanidine synthesis (LC/MS m/z 277.33 [M+H⁺])

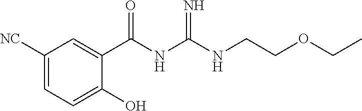 [SMILES: CCOCCNC(=N)NC(=O)c1cc(C#N)ccc1O]

This compound was prepared using General Procedure B of acylguanidine synthesis (LC/MS m/z 277.33 [M+H⁺])

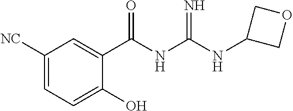 [SMILES: N#Cc1ccc(O)c(C(=O)NC(=N)NC2COC2)c1]

This compound was prepared using General Procedure B of acylguanidine synthesis (LC/MS m/z 261.28 [M+H⁺])

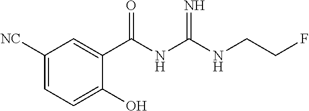 [SMILES: N#Cc1ccc(O)c(C(=O)NC(=N)NCCF)c1]

This compound was prepared using General Procedure B of acylguanidine synthesis (LC/MS m/z 251.29 [M+H⁺])

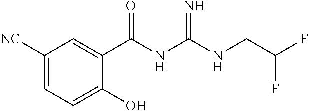 [SMILES: N#Cc1ccc(O)c(C(=O)NC(=N)NCC(F)F)c1]

This compound was prepared using General Procedure B of acylguanidine synthesis (LC/MS m/z 269.27 [M+H⁺])

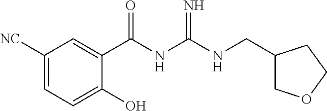 [SMILES: N#Cc1ccc(O)c(C(=O)NC(=N)NCC2CCOC2)c1]

This compound was prepared using General Procedure B of acylguanidine synthesis (LC/MS m/z 289.37 [M+H⁺])

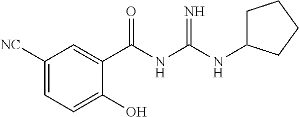 [SMILES: N#Cc1ccc(O)c(C(=O)NC(=N)NC2CCCC2)c1]

This compound was prepared using General Procedure B of acylguanidine synthesis (LC/MS m/z 273.35 [M+H⁺])

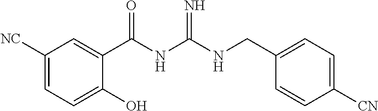 [SMILES: N#Cc1ccc(CNC(=N)NC(=O)c2cc(C#N)ccc2O)cc1]

This compound was prepared using General Procedure B of acylguanidine synthesis (LC/MS m/z 320.38 [M+H⁺])

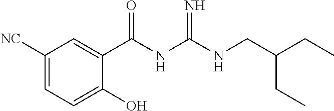 [SMILES: CCC(CC)CNC(=N)NC(=O)c1cc(C#N)ccc1O]

This compound was prepared using General Procedure C of acylguanidine synthesis (LC/MS m/z 289.41 [M+H⁺])

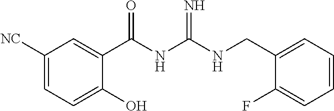 [SMILES: N#Cc1ccc(O)c(C(=O)NC(=N)NCc2ccccc2F)c1]

This compound was prepared using General Procedure C of acylguanidine synthesis (LC/MS m/z 313.38 [M+H⁺])

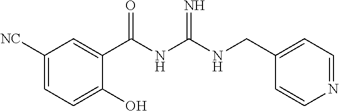 [SMILES: N#Cc1ccc(O)c(C(=O)NC(=N)NCc2ccncc2)c1]

This compound was prepared using General Procedure C of acylguanidine synthesis (LC/MS m/z 296.38 [M+H⁺])

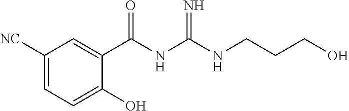 [SMILES: N#Cc1ccc(O)c(C(=O)NC(=N)NCCCO)c1]

This compound was prepared using General Procedure C of acylguanidine synthesis (LC/MS m/z 263.29 [M+H⁺])

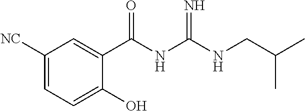 [SMILES: CC(C)CNC(=N)NC(=O)c1cc(C#N)ccc1O]

This compound was prepared using General Procedure B of acylguanidine synthesis (LC/MS m/z 261.33 [M+H⁺])

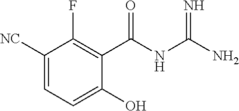 [SMILES: N#Cc1ccc(O)c(C(=O)NC(=N)N)c1F]

This compound was prepared using General Procedure A of acylguanidine synthesis, Method B (LC/MS m/z 223.27 [M+H⁺])

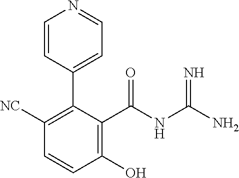 [SMILES: N#Cc1ccc(O)c(C(=O)NC(=N)N)c1-c1ccncc1]

This compound was prepared using General Procedure A of acylguanidine synthesis, Method B (LC/MS m/z 282.33 [M+H⁺])

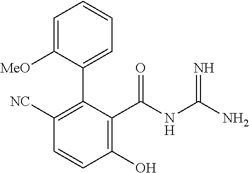 [SMILES: COc1ccccc1-c1c(C#N)ccc(O)c1C(=O)NC(=N)N]

This compound was prepared using General Procedure A of acylguanidine synthesis, Method B (LC/MS m/z 311.34 [M+H⁺])

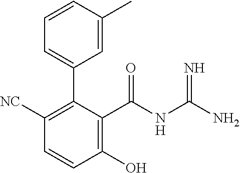 [SMILES: Cc1cccc(-c2c(C#N)ccc(O)c2C(=O)NC(=N)N)c1]

This compound was prepared using General Procedure A of acylguanidine synthesis, Method B (LC/MS m/z 295.36 [M+H⁺])

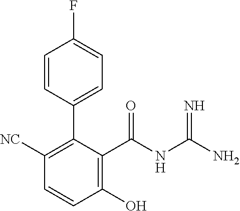 [SMILES: N#Cc1ccc(O)c(C(=O)NC(=N)N)c1-c1ccc(F)cc1]

This compound was prepared using General Procedure A of acylguanidine synthesis, Method B (LC/MS m/z 299.37 [M+H⁺])

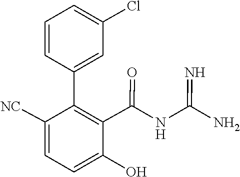 [SMILES: N#Cc1ccc(O)c(C(=O)NC(=N)N)c1-c1cccc(Cl)c1]

This compound was prepared using General Procedure A of acylguanidine synthesis, Method B (LC/MS m/z 315.35 [M+H⁺])

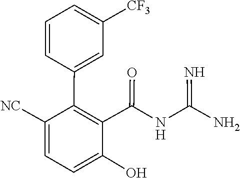 [SMILES: N#Cc1ccc(O)c(C(=O)NC(=N)N)c1-c1cccc(C(F)(F)F)c1]

This compound was prepared using General Procedure A of acylguanidine synthesis, Method B (LC/MS m/z 349.36 [M+H⁺])

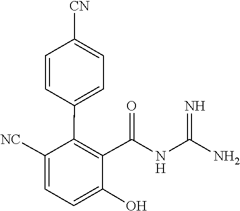 [SMILES: N#Cc1ccc(-c2c(C#N)ccc(O)c2C(=O)NC(=N)N)cc1]

This compound was prepared using General Procedure A of acylguanidine synthesis, Method B (LC/MS m/z 306.34 [M+H⁺])

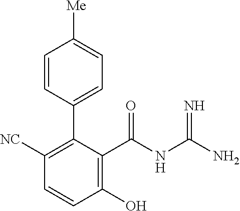 [SMILES: Cc1ccc(-c2c(C#N)ccc(O)c2C(=O)NC(=N)N)cc1]

This compound was prepared using General Procedure A of acylguanidine synthesis, Method B (LC/MS m/z 295.32 [M+H⁺])

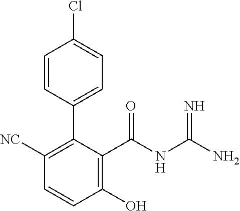 [SMILES: N#Cc1ccc(O)c(C(=O)NC(=N)N)c1-c1ccc(Cl)cc1]

This compound was prepared using General Procedure A of acylguanidine synthesis, Method B (LC/MS m/z 315.32 [M+H⁺])

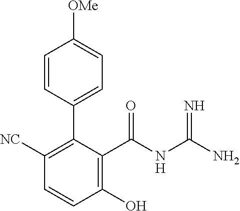 [SMILES: COc1ccc(-c2c(C#N)ccc(O)c2C(=O)NC(=N)N)cc1]

This compound was prepared using General Procedure A of acylguanidine synthesis, Method B (LC/MS m/z 311.30 [M+H⁺])

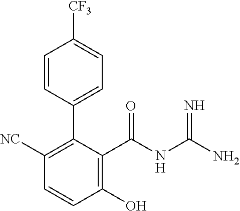 [SMILES: N#Cc1ccc(O)c(C(=O)NC(=N)N)c1-c1ccc(C(F)(F)F)cc1]

This compound was prepared using General Procedure A of acylguanidine synthesis, Method B (LC/MS m/z 349.32 [M+H⁺])

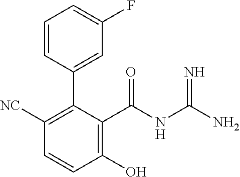 [SMILES: N#Cc1ccc(O)c(C(=O)NC(=N)N)c1-c1cccc(F)c1]

This compound was prepared using General Procedure A of acylguanidine synthesis, Method B (LC/MS m/z 299.30 [M+H⁺])

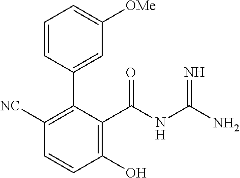 [SMILES: COc1cccc(-c2c(C#N)ccc(O)c2C(=O)NC(=N)N)c1]

This compound was prepared using General Procedure A of acylguanidine synthesis, Method B (LC/MS m/z 311.37 [M+H⁺])

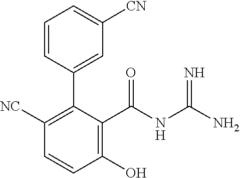 [SMILES: N#Cc1cccc(-c2c(C#N)ccc(O)c2C(=O)NC(=N)N)c1]

This compound was prepared using General Procedure A of acylguanidine synthesis, Method B (LC/MS m/z 306.37 [M+H⁺])

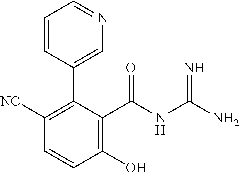 [SMILES: N#Cc1ccc(O)c(C(=O)NC(=N)N)c1-c1cccnc1]

This compound was prepared using General Procedure A of acylguanidine synthesis, Method B (LC/MS m/z 282.30 [M+H⁺])

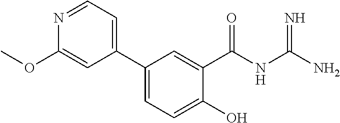 [SMILES: COc1cc(-c2ccc(O)c(C(=O)NC(=N)N)c2)ccn1]

This compound was prepared using General Procedure A of acylguanidine synthesis, Method A (LC/MS m/z 287.40 [M+H⁺]).

### Example 4: General Procedures for the Synthesis of Representative Compounds of the Invention

### General Procedure 1

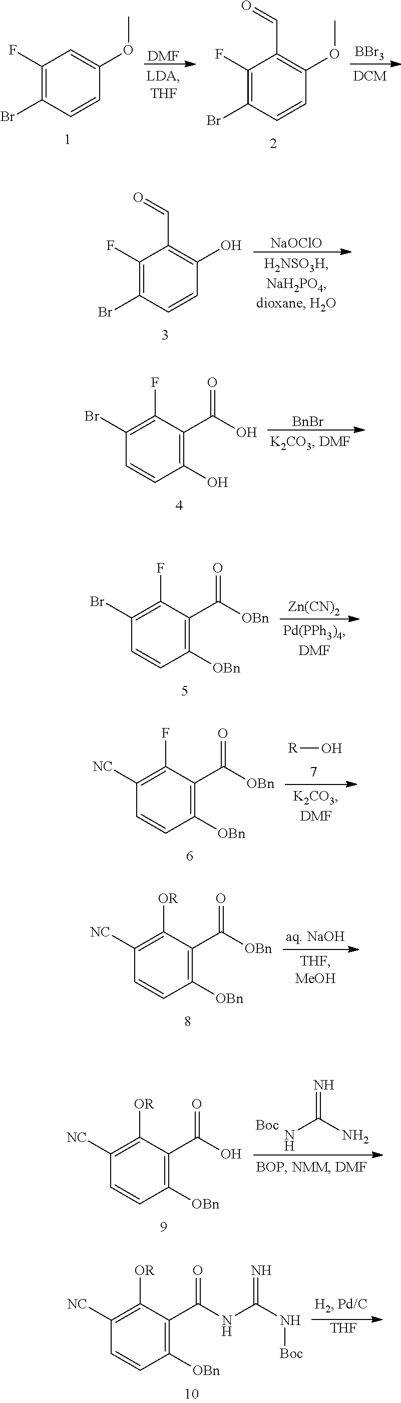

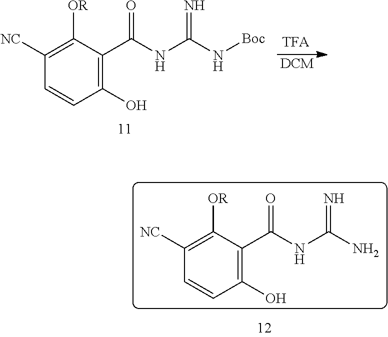

### Step 1: Synthesis of 3-bromo-2-fluoro-6-methoxybenzaldehyde (2)

LDA (73 mL, 2 M in THF) was added dropwise to a solution of compound 1 (25 g, 121.94 mmol) in THF (250 mL) at −78° C. under N₂ atmosphere. The mixture was stirred at −78° C. for 1 hour before anhydrous DMF (10.69 g, 146.32 mmol) was added. The resulting mixture was stirred at −78° C. for additional 30 minutes. The mixture was then diluted with ice/H₂O and extracted with EtOAc twice. The combined organic layers were washed with brine, dried over anhydrous Na₂SO₄, filtered and concentrated under vacuum. The residue was purified by column chromatography on silica gel (eluted with Petroleum Ether:EtOAc=20:1 to 5:1) to give compound 2 (23.0 g, 80.94% yield) as a yellow solid. LC/MS (ESI) m/z: 233/235 (M+H)⁺.

### Step 2: Synthesis of 3-bromo-2-fluoro-6-hydroxybenzaldehyde (3)

BBr₃ (25.80 g, 102.99 mmol) was added dropwise to a solution of 3-bromo-2-fluoro-6-methoxybenzaldehyde (12 g, 51.49 mmol) in anhydrous DCM (120 mL) at −50° C. under N₂ atmosphere. The resulting mixture was slowly warmed to room temperature and stirred for 1 hour. The mixture was then quenched with ice/H₂O and extracted with DCM twice. The combined organic layers were washed with brine, dried over anhydrous Na₂SO₄, filtered and concentrated under vacuum to give compound 3 (10.37 g, 91.98% yield) as a yellow solid. LC/MS (ESI) m/z: 217/219 (M−H)⁻.

### Step 3: Synthesis of 3-bromo-2-fluoro-6-hydroxybenzoic Acid (4)

H₂NSO₃H (6.89 g, 71.05 mmol) and NaH₂PO₄ (22.16 g, 184.72 mmol) were added to a solution of crude 3-bromo-2-fluoro-6-hydroxybenzaldehyde (10.37 g, 47.36 mmol) in dioxane (100 mL) at 0° C. followed by a solution of NaOClO (5.57 g, 61.57 mmol) in H₂O (100 mL) dropwise. The resulting mixture was stirred for 30 minutes at 0° C. and then diluted with H₂O and extracted with EtOAc twice. The combined organic layers were washed with brine, dried over anhydrous Na₂SO₄, filtered and concentrated under vacuum to give compound 4 (11.10 g, 99.72% yield) as a yellow solid. LC/MS (ESI) m/z: 233/235 (M−H)⁻.

### Step 4: Synthesis of benzyl 6-(benzyloxy)-3-bromo-2-fluorobenzoate (5)

Potassium carbonate (32.59 g, 236.16 mmol) and benzylbromide (24.23 g, 141.70 mmol) were added to a solution of crude 3-bromo-2-fluoro-6-hydroxybenzoic acid (11.10 g, 47.23 mmol) in DMF (100 mL) at 0° C. The resulting mixture was stirred at room temperature for 16 hours. The mixture was then diluted with H₂O and extracted with EtOAc twice. The combined organic layers were washed with saturated aq. NH₄Cl solution and brine, dried over anhydrous Na₂SO₄, filtered and concentrated under vacuum. The residue was purified by column chromatography on silica gel (Petroleum Ether:EtOAc=80:1 to 40:1) to give compound 5 (12.14 g, 61.92% yield) as a yellow solid. LC/MS (ESI) m/z: 415/417 (M+H)⁺.

### Step 5: Synthesis of benzyl 6-(benzyloxy)-3-cyano-2-fluorobenzoate (6)

Zinc cyanide (0.51 g, 4.33 mmol) and Pd(PPh₃)₄ (0.42 g, 0.36 mmol) were added to a solution of 6-(benzyloxy)-3-bromo-2-fluorobenzoate (1.5 g, 3.61 mmol) in DMF (10 mL) under N₂ atmosphere. The resulting mixture was stirred at 135° C. for 30 minutes under microwave. The mixture was then diluted with EtOAc and filtered. The filtrate was washed with water, saturated aq. NH₄Cl solution and brine, dried over anhydrous Na₂SO₄, filtered and concentrated under vacuum. The residue was purified by column chromatography on silica gel (Petroleum Ether:EtOAc=30:1 to 10:1) to give compound 6 (0.5 g, 38.30% yield) as a light yellow solid. LC/MS (ESI) m/z: 362 (M+H)⁺.

### Step 6: Synthesis of Compound 8

Potassium carbonate (5.0 eq) was added to a solution of benzyl 6-(benzyloxy)-3-cyano-2-fluorobenzoate (1.0 eq) in DMF followed by the appropriate phenol (3.0 eq). The resulting mixture was stirred at room temperature for 16 hours. The mixture was then diluted with H₂O and extracted with EtOAc twice. The combined organic layers were washed with saturated aq. NH₄Cl solution and brine, dried over anhydrous Na₂SO₄, filtered and concentrated under vacuum. The residue was purified by column chromatography on silica gel (Petroleum Ether:EtOAc=30:1 to 10:1) to give compound 8.

### Step 7: Synthesis of Compound 9

Aqueous. NaOH (2 M, 8.0 eq) was added to a solution of compound 8 (1.0 eq) in MeOH/THF (1:1, v/v) at 0° C. The reaction mixture was stirred at 70° C. overnight. The mixture was then cooled to room temperature and washed with Et₂O. The aqueous layer was separated, acidified to pH=5 with 0.5 M aq. HCl solution and extracted with EtOAc twice. The organic layers were washed with brine, dried over anhydrous Na₂SO₄, filtered and concentrated under vacuum to give compound 9.

### Step 8: Synthesis of Compound 10

NMM (4.0 eq) and PyBOP (1.4 eq) were added to a mixture of compound 9 (1.0 eq) and Boc-guanidne (2.6 eq) in DMF, and the resulting mixture was stirred at room temperature overnight under N₂ atmosphere. The mixture was then diluted with H₂O and extracted with EtOAc twice. The combined organic layers were washed with saturated aq. NH₄Cl solution and brine, dried over anhydrous Na₂SO₄, filtered and concentrated under vacuum. The residue was purified by column chromatography on silica gel (Petroleum Ether:EtOAc=10:1 to 4:1) to give compound 10.

### Step 9: Synthesis of Compound 11

10% Pd/C (1.0 eq) was added to a solution of compound 10 (1.0 eq) in THF and the mixture was degassed under N₂ atmosphere for three times and stirred under H₂ atmosphere at room temperature for 30 minutes. The mixture was then filtered and the filtrate was concentrated under vacuum. The residue was purified by column chromatography on silica gel (Petroleum Ether:EtOAc=10:1 to 5:1) to give compound 11.

### Step 10: Synthesis of Compound 12

TFA (1 ml) was added to a solution of compound 11 (1.0 eq) in DCM (1 mL) at 0° C. and the reaction was stirred at room temperature for 1 hour. The mixture was then concentrated under vacuum and the residue was purified via prep-HPLC (C18, 0% to 50% acetonitrile in H₂O with 0.1% NH₃.H₂O) to give the final compound 12.

### Synthesis of A69

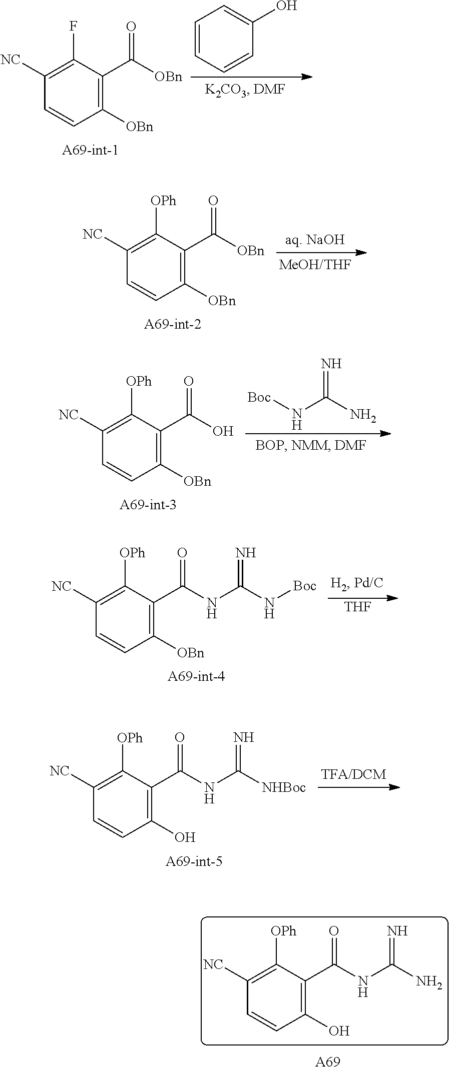

### Step 1: Synthesis of Compound A69-Int-2

Potassium carbonate (592 mg, 4.29 mmol) was added to a mixture of compound A69-int-1 (310 mg, 0.86 mmol) and phenol (242.21 mg, 2.57 mmol) in DMF (5 mL). The resulting mixture was stirred at room temperature overnight. The mixture was then diluted with H₂O and extracted with EtOAc twice. The combined organic layers were washed with saturated aq. NH₄Cl solution and brine, dried over anhydrous Na₂SO₄, filtered and concentrated under vacuum. The residue was purified by column chromatography on silica gel (Petroleum Ether:EtOAc=30:1 to 10:1) to give compound A69-int-2 (356 mg, 95.29% yield) as a yellow solid. LC/MS (ESI) m/z: 436 (M+H)⁺.

### Step 2: Synthesis of Compound A69-Int-3

A solution of NaOH (196 mg, 4.91 mmol) in H₂O (10 mL) was added to a solution of compound A69-int-2 (356 mg, 0.82 mmol) in MeOH (10 mL) and THF (10 mL) at 0° C. The mixture was stirred at 70° C. overnight. The mixture was then washed with Et₂O and the aqueous layer was separated, acidified with 0.5 M aq. HCl solution to pH=5 and extracted with EtOAc twice. The combined organic layers were washed with brine, dried over anhydrous Na₂SO₄, filtered and concentrated under vacuum to give compound A69-int-3 (280 mg, 99.18% yield) as a yellow solid without any further purification. LC/MS (ESI) m/z: 344 (M−H)⁻.

### Step 3: Synthesis of Compound A69-Int-4

NMM (491 mg, 4.86 mmol) and PyBOP (573 mg, 1.30 mmol) were added to a mixture of compound A69-int-3 (280 mg, 0.81 mmol) and Boc-guanidine (335 mg, 2.11 mmol) in DMF (4 mL). The resulting mixture was stirred at room temperature overnight under N₂ atmosphere. The mixture was then diluted with H₂O and extracted with EtOAc twice. The combined organic layers were washed with saturated aq. NH₄Cl solution and brine, dried over anhydrous Na₂SO₄, filtered and concentrated under vacuum. The residue was purified by column chromatography on silica gel (Petroleum Ether:EtOAc=10:1 to 4:1) to give compound A69-int-4 (390 mg, 98.87% yield) as a white solid. LC/MS (ESI) m/z: 487 (M+H)⁺.

### Step 4: Synthesis of Compound A69-Int-5

10% Pd/C (210 mg) was added to a solution of compound A69-int-4 (210 mg, 0.43 mmol) in THF (8 mL). The mixture was degassed under N₂ atmosphere for three times and stirred under 20 psi H₂ at room temperature for 30 minutes. The mixture was then filtered and the filtrate was concentrated under vacuum. The residue was purified by column chromatography on silica gel (Petroleum Ether:EtOAc=10:1 to 5:1) to give compound A69-int-5 (126 mg, 73.65% yield) as a white solid. LC/MS (ESI) m/z: 397 (M+H)⁺.

### Step 5: Synthesis of Compound A69

TFA (3 ml) was added dropwise to a solution of compound A69-int-5 (126 mg, 0.32 mmol) in DCM (3 mL) at 0° C. The reaction mixture was stirred at room temperature for 1 hour and then concentrated under vacuum. The residue was purified by prep-HPLC (C18, 0% to 50% acetonitrile in H₂O with 0.1% NH₃.H₂O) to give compound A69 (23.7 mg, 25.16% yield) as a white solid. LC/MS (ESI) m/z: 297 (M+H)⁺. ¹H NMR (400 MHz, DMSO-d₆) δ 8.15 (br s, 2H), 7.56 (d, J=8.9 Hz, 1H), 7.27 (t, J=7.8 Hz, 2H), 6.97 (t, J=7.1 Hz, 1H), 6.75 (d, J=8.5 Hz, 2H), 6.67 (d, J=8.9 Hz, 1H).

The compounds in the table below were prepared from the appropriate starting materials described previously or commercially available using the above general procedure 1.

| Structure | Name |
|---|---|
| 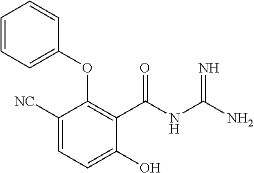 [SMILES: N#Cc1ccc(O)c(C(=O)NC(=N)N)c1Oc1ccccc1] | N-carbamimidoyl-3-cyano-6-hydroxy-2- phenoxybenzamide LC-MS: m/z 297 (M + H)⁺. 1H NMR (400 MHz, DMSO) δ 8.15 (br, 2H), 7.56 (d, J = 8.9 Hz, 1H), 7.27 (t, J = 7.8 Hz, 2H), 6.97 (t, J = 7.1 Hz, 1H), 6.75 (d, J = 8.5 Hz, 2H), 6.67 (d, J = 8.9 Hz, 1H). |
| 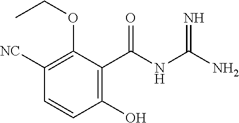 [SMILES: CCOc1c(C#N)ccc(O)c1C(=O)NC(=N)N] | N-carbamimidoyl-3-cyano-2-ethoxy-6- hydroxybenzamide LC-MS: m/z 249 (M + H)⁺. 1H NMR (400 MHz, DMSO) δ 8.49 (br, 2H), 7.35 (d, J = 8.8 Hz, 1H), 6.42 (d, J = 8.8 Hz, 1H), 4.02 (q, J = 7.0 Hz, 2H), 1.31 (t, J = 7.0 Hz, 3H). |
| 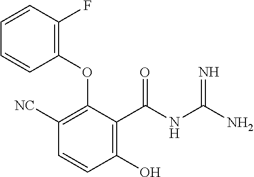 [SMILES: N#Cc1ccc(O)c(C(=O)NC(=N)N)c1Oc1ccccc1F] | N-carbamimidoyl-3-cyano-2-(2-fluorophenoxy)-6- hydroxybenzamide LC-MS: m/z 315 (M + H)⁺. 1H NMR (400 MHz, DMSO) δ 8.44 (br s, 2H), 7.60 (d, J = 8.9 Hz, 1H), 7.32 (br s, 1H) 7.30-7.20 (m, 1H), 7.03-6.95 (m, 2H), 6.71 (d, J = 8.9 Hz, 1H), 6.58-6.52 (m, 1H). |
| 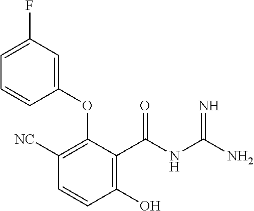 [SMILES: N#Cc1ccc(O)c(C(=O)NC(=N)N)c1Oc1cccc(F)c1] | N-carbamimidoyl-3-cyano-2-(3-fluorophenoxy)-6- hydroxybenzamide LC-MS: m/z 315 (M + H)⁺. 1H NMR (400 MHz, DMSO) δ 8.45 (br s, 2H), 7.60 (d, J = 8.9 Hz, 1H), 7.38 (br s, 1H), 7.28-7.17 (m, 1H), 6.83 (td, J = 8.3, 2.1 Hz, 1H), 6.71 (d, J = 8.9 Hz, 1H), 6.65 (dt, J = 10.7, 2.4 Hz, 1H), 6.57 (dd, J = 8.2, 2.2 Hz, 1H). |
| 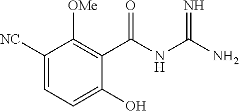 [SMILES: COc1c(C#N)ccc(O)c1C(=O)NC(=N)N] | N-carbamimidoyl-3-cyano-6-hydroxy-2- methoxybenzamide LC-MS: m/z 235 (M + H)⁺. 1H NMR (400 MHz, DMSO) δ 7.96 (br s, 3H), 7.46 (d, J = 8.8 Hz, 1H), 6.55 (d, J = 8.8 Hz, 1H), 3.82 (s, 3H). |

### General Procedure 2

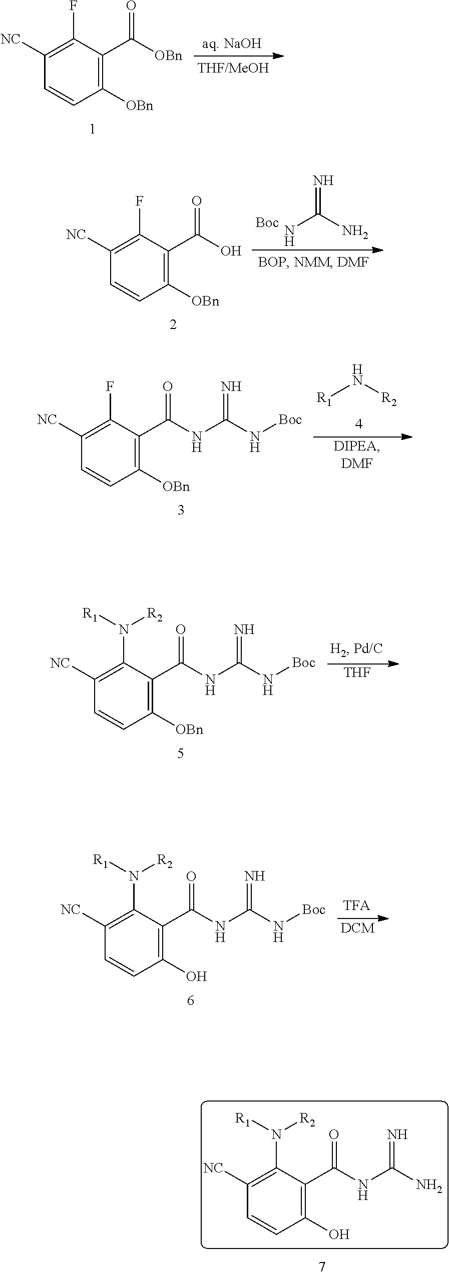

### Step 1: Synthesis of Compound 2

A solution of NaOH (521 mg, 13.03 mmol) in H₂O (10 mL) was added at 0° C. to a solution of compound 1 (942 mg, 2.61 mmol) in MeOH (10 mL) and THF (10 mL). The mixture was stirred at room temperature for 16 hours and then washed with Et₂O. The aqueous layer was acidified with 0.5M aq. HCl solution and extracted with EtOAc twice. The organic layers were washed with brine, dried over anhydrous Na₂SO₄, filtered and concentrated under vacuum to give compound 2 (648 mg, 91.65% yield) as a yellow solid without any further purification. LC/MS (ESI) m/z: 270 (M−H)⁻.

### Step 2: Synthesis of Compound 3

NMM (1448 mg, 14.33 mmol) was added followed by PyBOP (1690 mg, 3.82 mmol) to a mixture of compound 2 (648 mg, 2.39 mmol) and Boc-guanidine (988 mg, 6.21 mmol) in DMF (10 mL). The resulting mixture was stirred at room temperature for 16 hours. The mixture was then diluted with H₂O and extracted with EtOAc twice. The combined organic layers were washed with saturated aq. NH₄Cl solution and brine, dried over anhydrous Na₂SO₄, filtered and concentrated under vacuum. The residue was purified by column chromatography on silica gel (Petroleum Ether:EtOAc=10:1 to 3:1) to give compound 3 (550 mg, 55.82% yield) as a white solid. LC/MS (ESI) m/z: 413 (M+H)⁺.

### Step 3: Synthesis of Compound 5

The appropriate amine 4 (6.0 eq) was added to a solution of compound 3 (1.0 eq) and DIPEA (8.0 eq) in DMF and the resulting mixture was stirred at room temperature for 40 hours. The mixture was then diluted with H₂O and extracted with EtOAc twice. The combined organic layers were washed with saturated aq. NH₄Cl solution and brine, dried over anhydrous Na₂SO₄, filtered and concentrated under vacuum. The residue was purified via prep-TLC to give compound 5.

### Step 4: Synthesis of Compound 6

10% Pd/C (1.0 eq) was added to a solution of compound 5 (1.0 eq) in THF, and the mixture was degassed under N₂ atmosphere for three times and stirred under H₂ atmosphere at room temperature for 30 minutes. The mixture was then filtered and the filtrate was concentrated under vacuum. The residue was purified by prep-TLC to give compound 6.

### Step 5: Synthesis of Compound 7

TFA (1 ml) was added dropwise at 0° C. to a solution of compound 6 (1.0 eq) in DCM (1 mL) and the reaction was stirred at room temperature for 1 hour. The mixture was then concentrated under vacuum and the residue was purified by prep-HPLC (C18, 0% to 50% acetonitrile in H₂O with 0.1% NH₃.H₂O) to give compound 7.

### Synthesis of A124

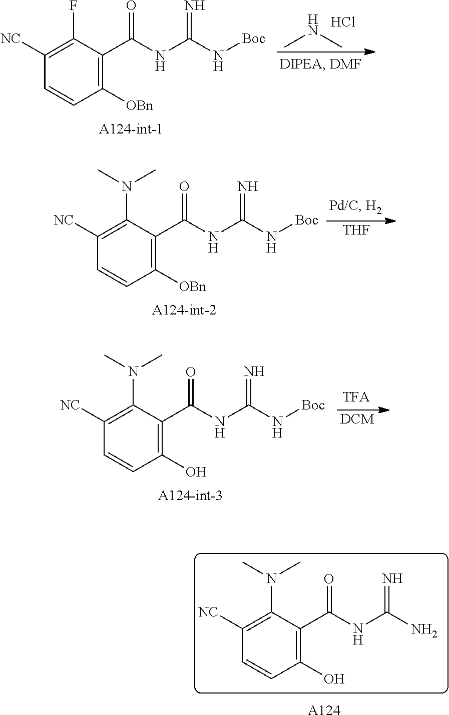

### Step 1: Synthesis of Compound A124-Int-2

Dimethylamine hydrochloride (178 mg, 2.18 mmol) was added to a mixture of compound A124-int-1 (150 mg, 0.36 mmol) and DIPEA (375 mg, 2.91 mmol) in DMF (3 mL). The resulting mixture was stirred at room temperature for 40 hours. The mixture was then diluted with H₂O and extracted with EtOAc twice. The combined organic layers were washed with saturated aq. NH₄Cl solution and brine, dried over anhydrous Na₂SO₄, filtered and concentrated under vacuum. The residue was purified by prep-TLC to give compound A124-int-2 (70 mg, 43.99% yield) as a white solid. LC/MS (ESI) m/z: 438 (M+H)⁺.

### Step 2: Synthesis of Compound A124-Int-3

10% Pd/C (70 mg) was added to a solution of compound A124-int-2 (70 mg, 0.16 mmol) in THF (5 mL), and the mixture was degassed under N₂ atmosphere for three times and stirred under 15 psi H₂ at room temperature for 30 minutes. The mixture was then filtered and the filtrate was concentrated under vacuum. The residue was purified by prep-TLC to give compound A124-int-3 (30 mg, 53.98% yield) as a yellow solid. LC/MS (ESI) m/z: 348 (M+H)⁺.

### Step 3: Synthesis of A124

TFA (3 ml) was added dropwise at 0° C. to a solution of compound A124-int-3 (30 mg, 0.09 mmol) in DCM (3 mL) and the reaction was stirred at room temperature for 2 hours. The mixture was then concentrated, diluted with saturated aq. NaHCO₃ and extracted with EtOAc twice. The combined organic layers were washed with brine, dried over anhydrous Na₂SO₄, filtered and concentrated under vacuum. The residue was purified via prep-HPLC (C18, 0% to 50% acetonitrile in H₂O with 0.1% NH₃.H₂O) to give compound A124 (5.5 mg, 25.75% yield) as a light yellow solid. LC/MS (ESI) m/z: 248 (M+H)⁺. ¹H NMR (400 MHz, DMSO-d6) δ 7.95 (s, 3H), 7.38 (d, J=8.6 Hz, 1H), 6.40 (d, J=8.6 Hz, 1H), 2.86 (s, 6H).

The compounds in the table below were prepared from the appropriate starting materials described previously or commercially available using the above general procedure 2.

| Structure | Name |
|---|---|
| 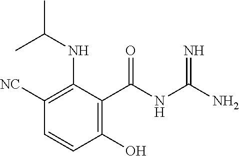 [SMILES: CC(C)Nc1c(C#N)ccc(O)c1C(=O)NC(=N)N] | N-carbamimidoyl-3-cyano-6-hydroxy-2- (isopropylamino)benzamide LC-MS: m/z 262 (M + H)⁺. 1H NMR (400 MHz, DMSO) δ 9.78 (d, J = 8.3 Hz, 1H), 8.28 (br s, 2H), 7.25 (br s, 1H), 7.22 (d, J = 8.8 Hz, 1H), 6.01 (d, J = 8.8 Hz, 1H), 4.47-4.33 (m, 1H), 1.20 (d, J = 6.2 Hz, 6H). |
| 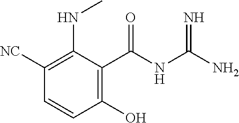 [SMILES: CNc1c(C#N)ccc(O)c1C(=O)NC(=N)N] | N-carbamimidoyl-3-cyano-6-hydroxy-2- (methylamino)benzamide LC-MS: m/z 234 (M + H)⁺. 1H NMR (400 MHz, DMSO) δ 9.80 (q, J = 4.8 Hz, 1H), 8.27 (br s, 2H), 7.26 (br s, 1H), 7.23 (d, J = 8.8 Hz, 1H), 6.00 (d, J = 8.8 Hz, 1H), 3.17 (d, J = 5.2 Hz, 3H). |
| 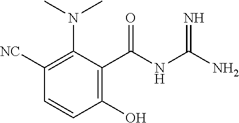 [SMILES: CN(C)c1c(C#N)ccc(O)c1C(=O)NC(=N)N] | N-carbamimidoyl-3-cyano-2-(dimethylamino)-6- hydroxybenzamide LC-MS: m/z 248 (M + H)⁺. 1H NMR (400 MHz, DMSO) δ 7.95 (br s, 3H), 7.38 (d, J = 8.6 Hz, 1H), 6.40 (d, J = 8.6 Hz, 1H), 2.86 (s, 6H). |
| 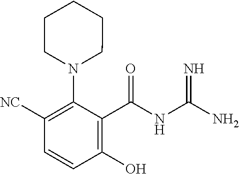 [SMILES: N#Cc1ccc(O)c(C(=O)NC(=N)N)c1N1CCCCC1] | N-carbamimidoyl-3-cyano-6-hydroxy-2-(piperidin-1- yl)benzamide LC-MS: m/z 288 (M + H)⁺. 1H NMR (400 MHz, DMSO) δ 15.00 (s, 1H), 8.32 (br s, 2H), 7.42 (d, J = 8.6 Hz, 1H), 7.13 (br s, 1H), 6.43 (d, J = 8.6 Hz, 1H), 3.15-3.06 (m, 4H), 1.66-1.52 (m, 6H). |

### General Procedure 3

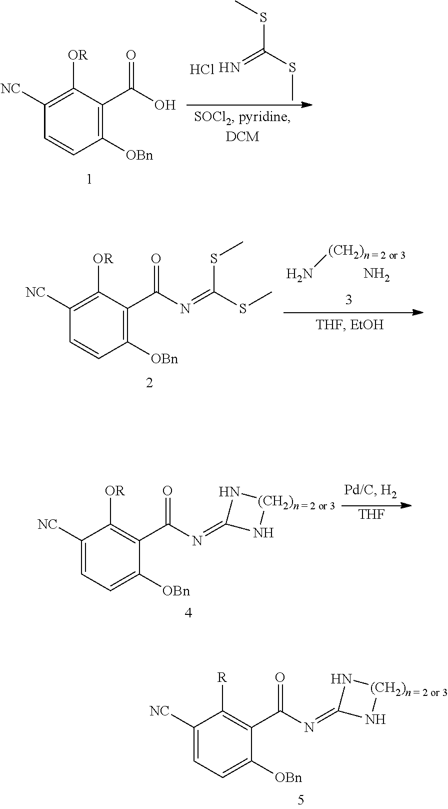

### Step 1: Synthesis of Compound 2

Thionyl chloride (10.0 eq) was added at 0° C. to a solution of compound 1 (1.0 eq) in DCM, and the reaction was stirred at 60° C. for 2 hours. The mixture was then cooled to room temperature and concentrated under vacuum. The crude residue was added to a mixture of dimethyl carbonimidodithioate (1.5 eq) and pyridine (1.5 eq) in DCM at 0° C. and the resulting mixture was stirred at room temperature for 30 minutes. The mixture was then concentrated under vacuum and the residue was purified by column chromatography on silica gel (Petroleum Ether:EtOAc=10:1 to 5:1) to give compound 2.

### Step 2: Synthesis of Compound 4

The appropriate diamine 3 (2.0 eq) was added to a solution of compound 2 (1.0 eq) in THF/EtOH (1:1, v/v) and the resulting mixture was stirred at 80° C. for 1 hour. The mixture was then cooled to room temperature and concentrated under vacuum to give a residue that was purified by column chromatography on silica gel (DCM:MeOH=80:1 to 40:1) to give compound 4.

### Step 3: Synthesis of Compound 5

10% Pd/C (1.0 eq) was added to a solution of compound 4 (1.0 eq) in THF and the mixture was degassed under N₂ atmosphere for three times and stirred under H₂ atmosphere at room temperature for 30 minutes. The mixture was then filtered and the filtrate was concentrated under vacuum. The residue was purified via prep-HPLC (C18, 0% to 50% acetonitrile in H₂O with 0.1% NH₃.H₂O) to give compound 5.

### Synthesis of A179

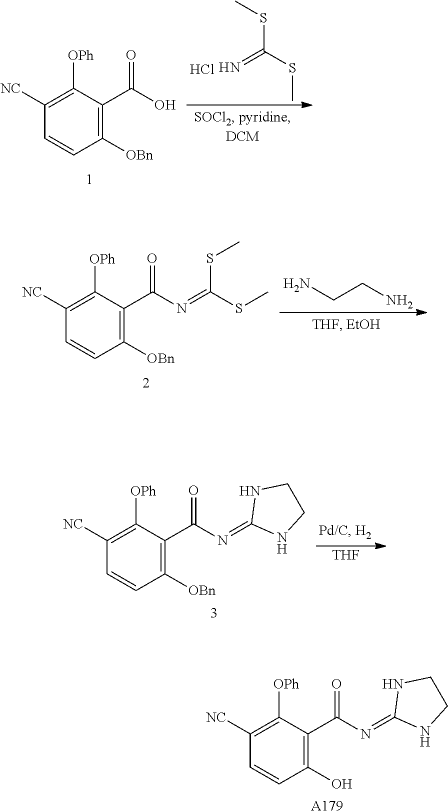

### Step 1: Synthesis of Compound 2

Thionyl chloride (0.40 mL, 5.50 mmol) was added at 0° C. to a solution of compound 1 (190 mg, 0.55 mmol) in DCM (5 mL). The reaction was stirred at 60° C. for 2 hours. The mixture was then cooled to room temperature and concentrated under vacuum. The crude residue was added to a mixture of dimethyl carbonimidodithioate (100 mg, 0.83 mmol) and pyridine (65 mg, 0.83 mmol) in DCM (5 mL) at 0° C. and the resulting mixture was stirred at room temperature for 30 minutes. The mixture was then concentrated under vacuum and the residue was purified by column chromatography on silica gel (Petroleum Ether:EtOAc=10:1 to 5:1) to give compound 2 (160 mg, 64.83% yield) as a white solid. LC/MS (ESI) m/z: 449 (M+H)⁺.

### Step 2: Synthesis of Compound 3

Ethylenediamine (0.02 mL, 0.36 mmol) was added to a solution of compound 2 (80 mg, 0.18 mmol) in THF (4 mL) and EtOH (4 mL) and the mixture was stirred at 80° C. for 1 hour. The mixture was then cooled to room temperature and concentrated under vacuum. The residue was purified by column chromatography on silica gel (DCM:MeOH=80:1 to 40:1) to give compound 3 (70 mg, 95.19% yield) as a white solid. LC/MS (ESI) m/z: 413 (M+H)⁺.

### Step 3: Synthesis of Compound A179

10% Pd/C (70 mg) was added to a solution of compound 3 (70 mg, 0.17 mmol) in THF (4 mL) and the mixture was degassed under N₂ atmosphere for three times and stirred under 15 psi H₂ at room temperature for 30 minutes. The mixture was then filtered and the filtrate was concentrated under vacuum. The residue was purified via prep-HPLC (C18, 0% to 50% acetonitrile in H₂O with 0.1% NH₃.H₂O) to give compound A179 (19.1 mg, 34.92% yield) as a white solid. LC/MS (ESI) m/z: 323 (M+H)⁺. ¹H NMR (400 MHz, DMSO-d₆) δ 8.68 (s, 2H), 7.67 (d, J=8.8 Hz, 1H), 7.28 (t, J=8.0 Hz, 2H), 6.98 (t, J=7.3 Hz, 1H), 6.77 (dd, J=18.1, 8.4 Hz, 3H), 3.54 (s, 4H).

The compounds in the table below were prepared from the appropriate starting materials described previously or commercially available using the above general procedure 3.

| Structure | Name |
|---|---|
| 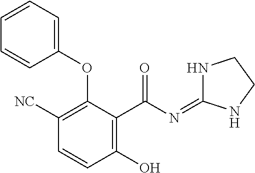 [SMILES: N#Cc1ccc(O)c(C(=O)N=C2NCCN2)c1Oc1ccccc1] | 3-cyano-6-hydroxy-N-(imidazolidin-2-ylidene)- 2-phenoxybenzamide LC-MS: m/z 323 (M + H)⁺. 1H NMR (400 MHz, DMSO) δ 8.68 (s, 2H), 7.67 (d, J = 8.8 Hz, 1H), 7.28 (t, J = 8.0 Hz, 2H), 6.98 (t, J = 7.3 Hz, 1H), 6.81-6.74 (m, 3H), 3.54 (s, 4H). |
| 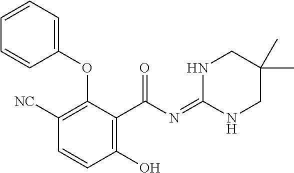 [SMILES: CC1(C)CNC(=NC(=O)c2c(O)ccc(C#N)c2Oc2ccccc2)NC1] | 3-cyano-N-(5,5-dimethyltetrahydropyrimidin- 2(1H)-ylidene)-6-hydroxy-2-phenoxybenzamide LC-MS: m/z 365 (M + H)⁺. 1H NMR (400 MHz, DMSO) δ 9.10 (s, 2H), 7.61 (d, J = 8.9 Hz, 1H), 7.28 (t, J = 8.0 Hz, 2H), 6.98 (t, J = 7.3 Hz, 1H), 6.77-6.71 (m, 3H), 2.97 (s, 4H), 0.95 (s, 6H). |
| 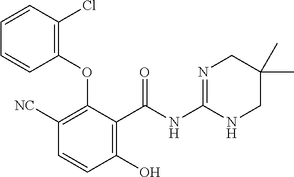 [SMILES: CC1(C)CN=C(NC(=O)c2c(O)ccc(C#N)c2Oc2ccccc2Cl)NC1] | 2-(2-chlorophenoxy)-3-cyano-N-(5,5-dimethyl- 1,4,5,6-tetrahydropyrimidin-2-yl)-6- hydroxybenzamide LC-MS: m/z 399 (M + H)⁺. 1H NMR (400 MHz, DMSO) δ 9.11 (s, 2H), 7.63 (d, J = 8.8 Hz, 1H), 7.49 (dd, J = 8.0, 1.6 Hz, 1H), 7.22-7.13 (m, 1H), 7.00 (td, J = 7.6, 1.2 Hz, 1H), 6.76 (d, J = 8.8 Hz, 1H), 6.51 (d, J = 7.2 Hz, 1H), 2.98 (s, 4H), 0.95 (s, 6H). |
| 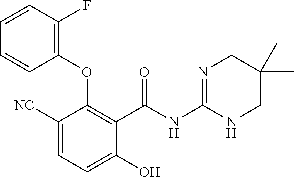 [SMILES: CC1(C)CN=C(NC(=O)c2c(O)ccc(C#N)c2Oc2ccccc2F)NC1] | 3-cyano-N-(5,5-dimethyl-1,4,5,6- tetrahydropyrimidin-2-yl)-2-(2-fluorophenoxy)- 6-hydroxybenzamide LC-MS: m/z 383 (M + H)⁺. 1H NMR (400 MHz, MeOD) δ 7.47 (dd, J = 8.9, 3.2 Hz, 1H), 7.17-7.09 (m, 1H), 6.99-6.91 (m, 2H), 6.71 (dd, J = 8.9, 3.0 Hz, 1H), 6.62-6.57 (m, 1H), 3.04 (s, 4H), 1.04 (s, 6H). |

### General Procedure 4

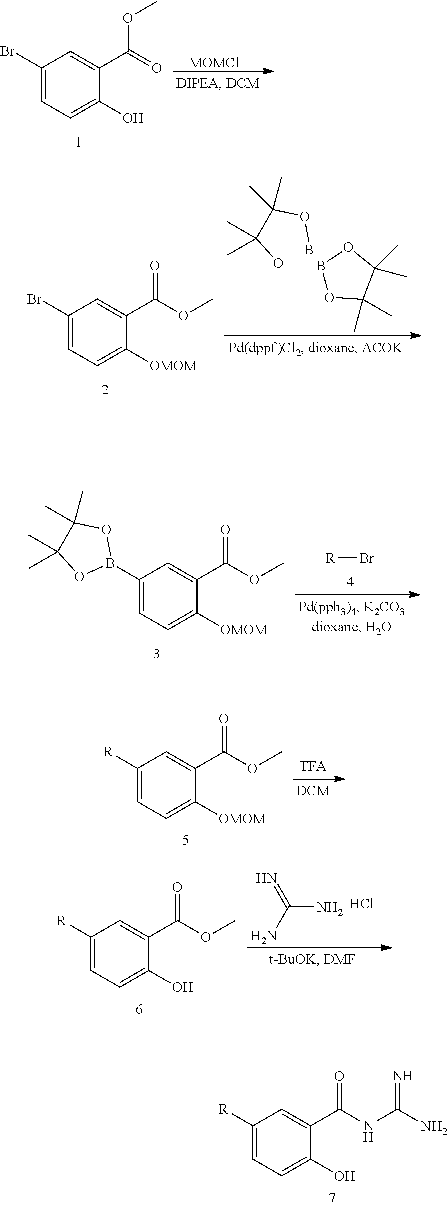

### Step 1: Synthesis of Compound 2

Hunig's base (1697 mg, 13.16 mmol) was added to a solution of compound 1 (760 mg, 3.29 mmol) in dry DCM (10 mL) at 0° C. under N₂ atmosphere followed by MOMCl (530 mg, 6.58 mmol). The resulting mixture was stirred at room temperature for 16 hours. The mixture was then poured into a saturated aq. NaHCO₃ solution and extracted with DCM twice. The combined organic layers were washed with brine, dried over Na₂SO₄, filtered and concentrated under vacuum. The residue was purified by column chromatography on silica gel (Petroleum Ether:EtOAc=30:1 to 10:1) to give compound 2 (466 mg, 51.50% yield) as a yellow oil. LC/MS (ESI) m/z: 275 (M+H)⁺.

### Step 2: Synthesis of Compound 3

Potassium acetate (107 mg, 1.09 mmol) and Pd(dppf)Cl₂ (40 mg, 0.05 mmol) were added to a mixture of compound 2 (150 mg, 0.55 mmol) and 4,4,4′,4′,5,5,5′,5′-octamethyl-2,2′-bi(1,3,2-dioxaborolane) (208 mg, 0.82 mmol) in dioxane (6 mL). The resulting mixture was stirred at 110° C. for 1.5 hours under N₂ atmosphere. The mixture was then diluted with H₂O and extracted with EtOAc twice. The combined organic layers were washed with brine, dried over anhydrous Na₂SO₄, filtered and concentrated under vacuum. The residue was purified by column chromatography on silica gel (Petroleum Ether:EtOAc=20:1 to 5:1) to give compound 3 (162 mg, 92.22% yield) as a yellow oil. LC/MS (ESI) m/z: 323 (M+H)⁺.

### Step 3: Synthesis of Compound 5

Potassium carbonate (2.0 eq) and Pd(PPh₃)₄ (0.1 eq) were added to a mixture of compound 3 (1.0 eq) and the appropriate pyridine bromide 4 (1.2 eq) in dioxane/H₂O (8:1). The mixture was stirred at 95° C. for 16 hours under N₂ atmosphere. The mixture was then diluted with H₂O and extracted with EtOAc twice. The combined organic layers were washed with brine, dried over anhydrous Na₂SO₄, filtered and concentrated under vacuum. The residue was purified by column chromatography on silica gel (Petroleum Ether:EtOAc=10:1 to 2:1) to give compound 5.

### Step 4: Synthesis of Compound 6

TFA (1:1, v/v) was added dropwise at 0° C. to a solution of compound 5 (1.0 eq) in DCM and the mixture was stirred at room temperature for 1 hour. The mixture was then concentrated, diluted with saturated aq. NaHCO₃ and extracted with EtOAc twice. The combined organic layers were washed with brine, dried over anhydrous Na₂SO₄, filtered and concentrated to give compound 6 without any further purification.

### Step 5: Synthesis of Compound 7

Potassium tert-butoxide (15.0 eq) was added to a solution of guanidine hydrochloride (12.0 eq) in DMF, and the mixture was stirred for 45 minutes at room temperature. Then a solution of compound 6 (1.0 eq) in DMF was added to the above solution and the resulting mixture was stirred at room temperature for 16 hours. The mixture was then diluted with H₂O and extracted with EtOAc twice. The combined organic layers were washed with saturated aq. NH₄Cl solution and brine, dried over anhydrous Na₂SO₄, filtered and concentrated under vacuum. The residue was triturated with MeOH and filtered to give compound 7.

### Synthesis of A57

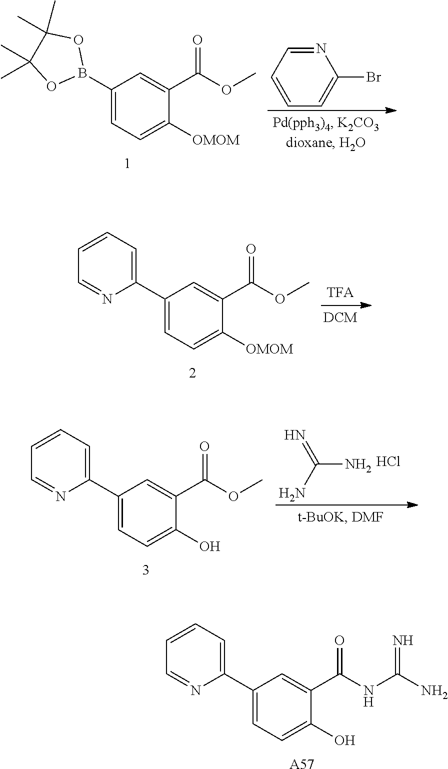

### Step 1: Synthesis of Compound 2

Potassium carbonate (146 mg, 1.06 mmol) was added to a mixture of compound 1 (170 mg, 0.53 mmol) and 2-bromopyridine (100 mg, 0.63 mmol) in dioxane (8 mL) and H₂O (1 mL) followed by Pd(pph₃)₄ (61 mg, 0.05 mmol). The resulting mixture was then stirred at 95° C. for 16 hours under N₂ atmosphere. The mixture was then diluted with H₂O and extracted with EtOAc twice. The combined organic layers were washed with brine, dried over anhydrous Na₂SO₄, filtered and concentrated under vacuum. The residue was purified by column chromatography on silica gel (Petroleum Ether:EtOAc=10:1 to 3:1) to give compound 2 (128 mg, 88.76% yield) as a colorless oil. LC/MS (ESI) m/z: 274 (M+H)⁺.

### Step 2: Synthesis of Compound 3

TFA (3 ml) was added dropwise at 0° C. to a solution of compound 2 (128 mg, 0.47 mmol) in DCM (3 mL) and the mixture was stirred at room temperature for 1 hour. The mixture was then concentrated, diluted with saturated aq. NaHCO₃ and extracted with EtOAc twice. The combined organic layers were washed with brine, dried over anhydrous Na₂SO₄, filtered and concentrated under vacuum to give compound 3 (107 mg, 99.65% yield) as a yellow solid. LC/MS (ESI) m/z: 230 (M+H)⁺.

### Step 3: Synthesis of Compound A57

Potassium tert-butoxide (785 mg, 7.01 mmol) was added to a solution of guanidine hydrochloride (535 mg, 5.60 mmol) in DMF (8 mL) and the resulting mixture was stirred at room temperature for 45 minutes. Then a solution of compound 3 (107 mg, 0.47 mmol) in DMF (2 mL) was added to the above reaction and the resulting mixture was stirred at room temperature for 16 hours. The reaction mixture was then diluted with H₂O and extracted with EtOAc twice. The combined organic layers were washed with saturated aq. NH₄Cl solution and brine, dried over anhydrous Na₂SO₄, filtered and concentrated under vacuum. The residue was triturated with MeOH and filtered to give compound A57 as a yellow solid (26.6 mg, 22.24% yield). LC/MS (ESI) m/z: 257 (M+H)⁺. ¹H NMR (400 MHz, DMSO) δ 15.21 (s, 1H), 8.60 (d, J=2.5 Hz, 2H), 8.40 (s, 1.5H), 8.04 (dd, J=8.6, 2.4 Hz, 1H), 7.85-7.79 (m, 2H), 7.28-7.23 (m, 1H), 7.09 (s, 1.5H), 6.88 (d, J=8.6 Hz, 1H).

The compounds in the table below were prepared from the appropriate starting materials described previously or commercially available using the above general procedure 4.

| Structure | Name |
|---|---|
| 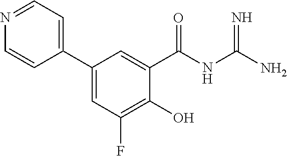 [SMILES: N=C(N)NC(=O)c1cc(-c2ccncc2)cc(F)c1O] | N-carbamimidoyl-3-fluoro-2-hydroxy-5-(pyridin-4- yl)benzamide LC-MS: m/z 275 (M + H)⁺. 1H NMR (400 MHz, DMSO) δ 8.69 (br s, 1H), 8.68 (d, J = 6.4 Hz, 2H), 8.22 (d, J = 2.2 Hz, 1H), 8.08 (d, J = 6.2 Hz, 2H), 7.93 (dd, J = 12.5, 2.4 Hz, 1H), 7.72 (br s, 1H). |
| 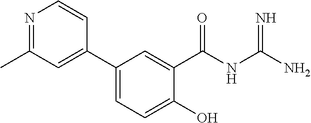 [SMILES: Cc1cc(-c2ccc(O)c(C(=O)NC(=N)N)c2)ccn1] | N-carbamimidoyl-2-hydroxy-5-(2-methylpyridin-4- yl)benzamide LC-MS: m/z 271 (M + H)⁺. 1H NMR (400 MHz, DMSO) δ 15.25 (s, 1H), 8.42 (d, J = 5.2 Hz, 1H), 8.23 (d, J = 1.6 Hz, 1H), 7.75 (dd, J = 8.5, 1.8 Hz, 1H), 7.49 (s, 1H), 7.41 (d, J = 5.1 Hz, 1H), 7.10 (br s, 2H), 6.90 (d, J = 8.5 Hz, 1H), 2.50 (s, 3H). |
| 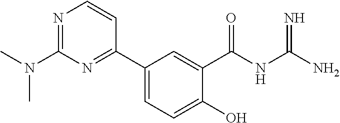 [SMILES: CN(C)c1nccc(-c2ccc(O)c(C(=O)NC(=N)N)c2)n1] | N-carbamimidoyl-5-(2-(dimethylamino)pyrimidin-4-yl)- 2-hydroxybenzamide LC-MS: m/z 301 (M + H)⁺. 1H NMR (400 MHz, DMSO) δ 15.53 (s, 1H), 8.64 (d, J = 2.3 Hz, 1H), 8.44 (br s, 1H), 8.32 (d, J = 5.2 Hz, 1H), 8.09 (dd, J = 8.6, 2.3 Hz, 1H), 7.10 (br s, 1H), 7.02 (d, J = 5.2 Hz, 1H), 6.88 (d, J = 8.6 Hz, 1H), 3.19 (s, 6H). |
| 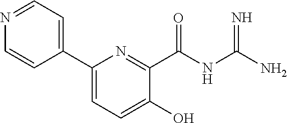 [SMILES: N=C(N)NC(=O)c1nc(-c2ccncc2)ccc1O] | N-carbamimidoyl-5-hydroxy-[2,4′-bipyridine]-6- carboxamide LC-MS: m/z 258 (M + H)⁺. 1H NMR (400 MHz, DMSO) δ 8.66 (d, J = 4.6 Hz, 1H), 8.25 (br s, 1H), 8.30-7.92 (m, 3H), 7.60 (br s, 1H), 7.43 (d, J = 7.8 Hz, 1H), 6.98 (s, 1H). |
| 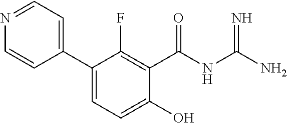 [SMILES: N=C(N)NC(=O)c1c(O)ccc(-c2ccncc2)c1F] | N-carbamimidoyl-2-fluoro-6-hydroxy-3-(pyridin-4- yl)benzamide LC-MS: m/z 275 (M + H)⁺. 1H NMR (400 MHz, DMSO) δ 8.58 (d, J = 4.8 Hz, 2H), 8.62 (br s, 2H), 7.50-7.44 (m, 3H), 7.19 (br s, 2H), 6.73 (d, J = 8.8 Hz, 1H). |
| 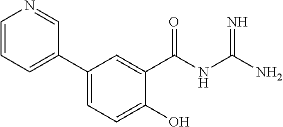 [SMILES: N=C(N)NC(=O)c1cc(-c2cccnc2)ccc1O] | N-carbamimidoyl-2-hydroxy-5-(pyridin-3-yl)benzamide LC-MS: m/z 257 (M + H)⁺. 1H NMR (400 MHz, DMSO) δ 15.10 (s, 1H), 8.82 (d, J = 2.0 Hz, 1H), 8.50 (dd, J = 4.7, 1.3 Hz, 1H), 8.42 (br s, 2H), 8.15 (d, J = 2.5 Hz, 1H), 8.03-7.95 (m, 1H), 7.69 (dd, J = 8.5, 2.5 Hz, 1H), 7.44 (dd, J = 7.9, 4.8 Hz, 1H), 7.11 (br s, 1H), 6.92 (d, J = 8.5 Hz, 1H). |
| 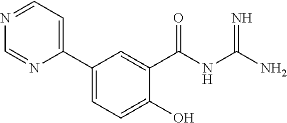 [SMILES: N=C(N)NC(=O)c1cc(-c2ccncn2)ccc1O] | N-carbamimidoyl-2-hydroxy-5-(pyrimidin-4- yl)benzamide LC-MS: m/z 258 (M + H)⁺. 1H NMR (400 MHz, DMSO) δ 15.75 (s, 1H), 9.14 (s, 1H), 8.76-8.72 (m, 2H), 8.45 (br s, 2H), 8.16 (dd, J = 8.7, 2.5 Hz, 1H), 7.95 (dd, J = 5.5, 1.3 Hz, 1H), 7.16 (br s, 1H), 6.92 (d, J = 8.7 Hz, 1H). |
| 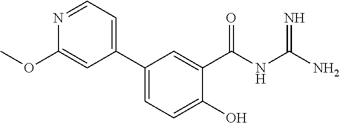 [SMILES: COc1cc(-c2ccc(O)c(C(=O)NC(=N)N)c2)ccn1] | N-carbamimidoyl-2-hydroxy-5-(2-methoxypyridin-4- yl)benzamide LC-MS: m/z 287 (M + H)⁺. 1H NMR (400 MHz, DMSO) δ 15.27 (s, 1H), 8.40 (br s, 2H), 8.20 (d, J = 2.5 Hz, 1H), 8.17 (d, J = 5.4 Hz, 1H), 7.75 (dd, J = 8.6, 2.5 Hz, 1H), 7.23 (dd, J = 5.4, 1.4 Hz, 1H), 7.10 (br s, 1H), 6.98 (s, 1H), 6.89 (d, J = 8.6 Hz, 1H), 3.88 (s, 3H). |
| 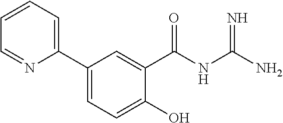 [SMILES: N=C(N)NC(=O)c1cc(-c2ccccn2)ccc1O] | N-carbamimidoyl-2-hydroxy-5-(pyridin-2-yl)benzamide LC-MS: m/z 257 (M + H)⁺. 1H NMR (400 MHz, DMSO) δ 15.21 (s, 1H), 8.60 (d, J = 2.5 Hz, 2H), 8.40 (br s, 2H), 8.04 (dd, J = 8.6, 2.4 Hz, 1H), 7.85-7.79 (m, 2H), 7.28-7.23 (m, 1H), 7.09 (br s, 1H), 6.88 (d, J = 8.6 Hz, 1H). |
| 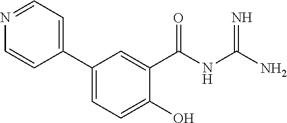 [SMILES: N=C(N)NC(=O)c1cc(-c2ccncc2)ccc1O] | N-carbamimidoyl-2-hydroxy-5-(pyridin-4-yl)benzamide LC-MS: m/z 257 (M + H)⁺. 1H NMR (400 MHz, DMSO) δ 15.31 (s, 1H), 8.56 (d, J = 6.0 Hz, 2H), 8.35 (br s, 2H), 8.25 (d, J = 2.5 Hz, 1H), 7.78 (dd, J = 8.6, 2.5 Hz, 1H), 7.62 (d, J = 6.1 Hz, 2H), 7.13 (br s, 1H), 6.92 (d, J = 8.5 Hz, 1H). |

### General Procedure 5

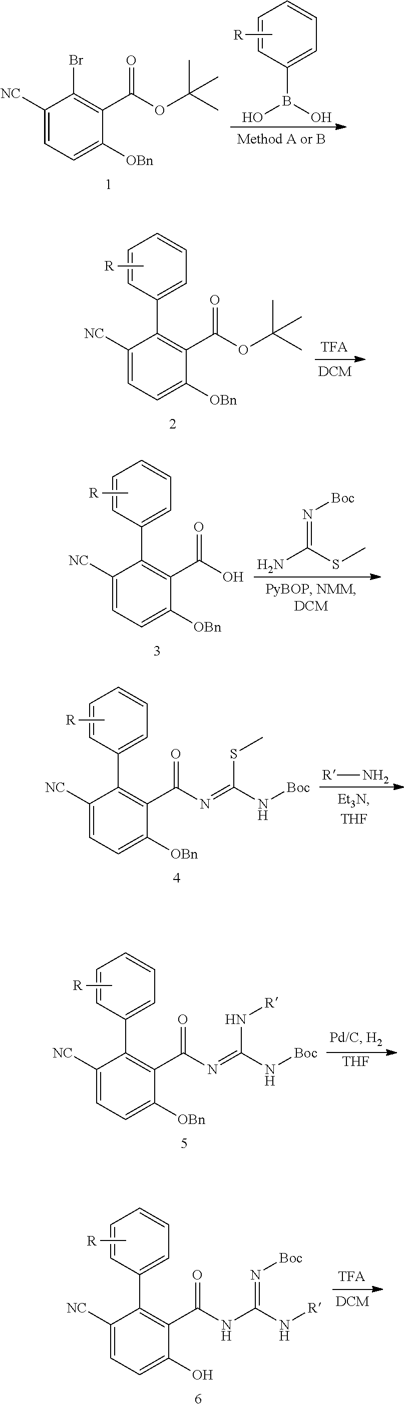

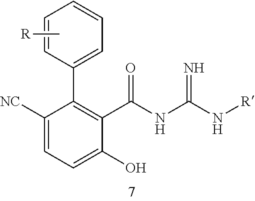

### Step 1: Synthesis of Compound 2

### Method A:

The appropriate phenylboronic acid (1.5 eq.) was added to a mixture of compound 1 (1 eq.) and K₂PO₃ (2.5 eq.) in 1,4-dioxane/H₂O (8:1) followed by Pd(OAc)₂ (0.1 eq.) and S-Phos (0.1 eq.) under N₂ atmosphere. The resulting mixture was degassed three times and stirred overnight at 95° C. under N₂ atmosphere. The mixture was then diluted with water and extracted with EtOAc twice. The combined organic layers were washed with brine, dried over anhydrous Na₂SO₄, filtered and concentrated under vacuum. The residue was purified by column chromatography on silica gel (eluted with Petroleum Ether:EtOAc=30:1 to 8:1) to give compound 2.

### Method B:

The appropriate phenylboronic acid (1.5 eq.) was added to a mixture of compound 1 (1 eq.) and potassium carbonate (2.5 eq.) in 1,4-dioxane/H₂O (8:1) followed by Pd(PPh₃)₄ (0.1 eq.) under N₂ atmosphere. The resulting mixture was degassed three times and stirred overnight at 95° C. under N₂ atmosphere. The mixture was then diluted with water and extracted with EtOAc twice. The combined organic layers were washed with brine, dried over anhydrous Na₂SO₄, filtered and concentrated under vacuum. The residue was purified by column chromatography on silica gel (eluted with Petroleum Ether:EtOAc=30:1 to 8:1) to give compound 2.

### Step 2: Synthesis of Compound 3

TFA (1:2 v/v) was added dropwise at 0° C. to a solution of compound 2 in DCM, and the reaction was stirred at room temperature for 2 hours. The reaction was then concentrated under vacuum and the residue was diluted with H₂O and extracted with DCM twice. The combined organic layers were washed with brine, dried over anhydrous Na₂SO₄, filtered and concentrated under vacuum. The residue was purified by column chromatography on silica gel (eluted with Petroleum Ether:EtOAc=10:1 to 3:1) to give compound 3.

### Step 3: Synthesis of Compound 4

tert-butyl amino(methylthio)methylenecarbamate (1.1 eq.) and PyBOP (1.5 eq.) were added to a solution of compound 3 (1.0 eq.) and NMM (5.0 eq.) in DCM and the resulting mixture was stirred at room temperature overnight under N₂ atmosphere. H₂O was added and the resulting mixture was extracted with EtOAc. The organic layer was separated, dried over anhydrous Na₂SO₄, filtered and concentrated under vacuum. The residue was purified by column chromatography on silica gel (eluted with Petroleum Ether:EA=8:1 to 2:1) to give compound 4.

### Step 4: Synthesis of Compound 5

The appropriate primary amine (1.1 eq.) was added to a solution of compound 4 (1.0 eq.) and TEA (5.0 eq.) in DCM, and the resulting mixture was stirred at room temperature for 1 hour. The mixture was then diluted with EtOAc and filtered. The filtrate was washed with brine, dried over anhydrous Na₂SO₄, filtered and concentrated under vacuum. The residue was purified by column chromatography on silica gel (eluted with Petroleum Ether:EtOAc=10:1 to 3:1) to give compound 5.

### Step 5: Synthesis of Compound 6

10% Pd/C (1:1, w/w) was added to a solution of compound 5 (1.0 eq.) in THF and the mixture was degassed under N₂ atmosphere for three times and stirred under 15 psi H₂ at room temperature for 30 minutes. The mixture was then filtered and the filtrate was concentrated under vacuum. The residue was purified by column chromatography on silica gel (eluted with Petroleum Ether:EtOAc=10:1 to 3:1) to give compound 6.

### Step 6: Synthesis of Compound 7

TFA (1:1, v/v) was added dropwise at 0° C. to a solution of compound 6 (1.0 eq.) in DCM, and the resulting solution was stirred at room temperature for 1 hour. The mixture was then concentrated under vacuum and the residue was solubilized in saturated aq. NaHCO₃ solution and extracted with EtOAc twice. The combined organic layers were washed with brine, dried over anhydrous Na₂SO₄, filtered and concentrated under vacuum. The residue was purified via prep-HPLC (C18, 0% to 50% acetonitrile in H₂O with 0.1% NH₃.H₂O) to give compound 7.

### Synthesis of A99

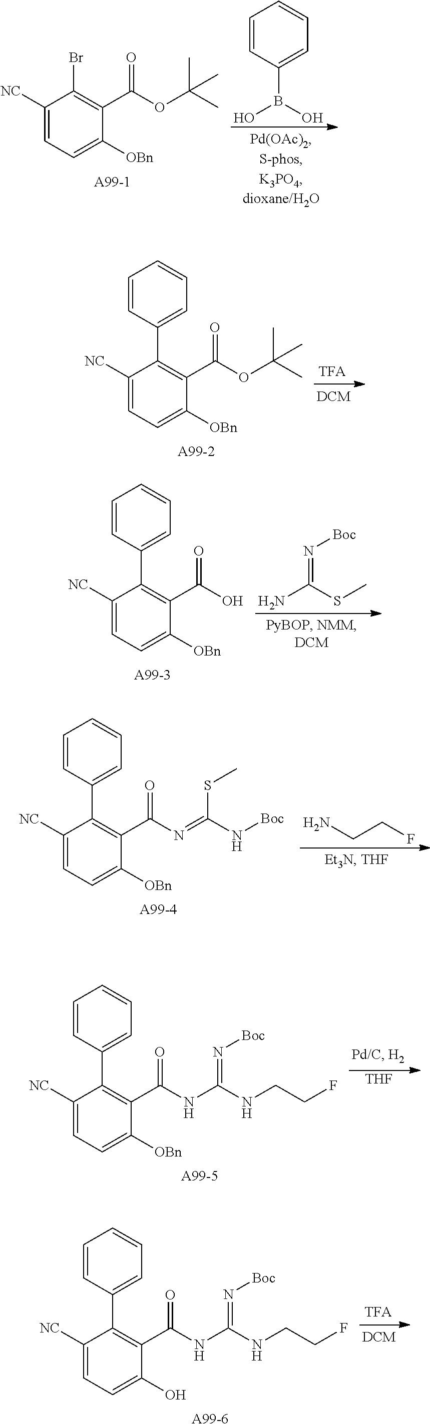

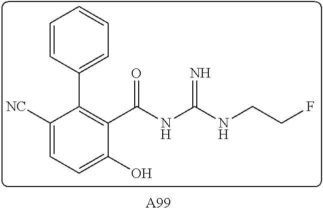

### Step 1: Synthesis of Compound A99-2

Phenylboronic acid (94 mg, 0.78 mmol) was added to a solution of compound A99-1 (200 mg, 0.52 mmol) and K₂PO₃ (273 mg, 1.31 mmol) in 1,4-dioxane (8 mL) and H₂O (1 mL) followed by Pd(OAc)₂ (12 mg, 0.05 mmol) and S-Phos (21 mg, 0.05 mmol) under N₂ atmosphere. The reaction mixture was then degassed three times and stirred overnight at 95° C. under N₂ atmosphere. The mixture was then diluted with water and extracted with EtOAc twice. The combined organic layers were washed with brine, dried over anhydrous Na₂SO₄, filtered and concentrated under vacuum. The residue was purified by column chromatography on silica gel (eluted with Petroleum Ether:EtOAc=30:1 to 8:1) to give compound A99-2 (190 mg, 0.49 mmol, 95.7% yield) as a yellow solid. LC/MS (ESI) m/z: 386 (M+H)⁺.

### Step 2: Synthesis of Compound A99-3

TFA (2 mL) was added dropwise at 0° C. to a solution of compound A-99-2 (190 mg, 0.49 mmol) in DCM (4 mL) and the reaction was stirred at room temperature for 2 hours. The mixture was then concentrated under vacuum and the residue was diluted with H₂O and extracted with DCM twice. The combined organic layers were washed with brine, dried over anhydrous Na₂SO₄, filtered and concentrated under vacuum. The residue was purified by column chromatography on silica gel (eluted with Petroleum Ether:EtOAc=10:1 to 3:1) to give compound A99-3 (155 mg, 0.47 mmol, 95.0% yield) as a yellow solid. LC/MS (ESI) m/z: 330 (M+H)⁺.

### Step 3: Synthesis of Compound A99-4

tert-butyl amino(methylthio)methylenecarbamate (99 mg, 0.52 mmol) and PyBOP (366 mg, 0.70 mmol) were added to a solution of compound A99-3 (155 mg, 0.47 mmol) and NMM (237 mg, 2.35 mmol) in DCM. The resulting mixture was stirred overnight at room temperature under N₂ atmosphere. The mixture was then washed with water and extracted with EtOAc. The organic layer was separated, dried over Na₂SO₄, filtered and concentrated under vacuum. The residue was purified by column chromatography on silica gel (eluted with Petroleum Ether:EtOAc=8:1 to 2:1) to give compound A99-4 (188 mg, 3.75 mmol, 79.6% yield) as a light yellow oil. LC/MS (ESI) m/z: 502 (M+H)⁺.

### Step 4: Synthesis of Compound A99-5

2-fluoroethanamine hydrochloride (18 mg, 0.18 mmol) was added to a solution of compound A99-4 (80 mg, 0.16 mmol) and Et₃N (81 mg, 0.80 mmol) in THF and the reaction was stirred at room temperature for 1 hour. The mixture was then diluted with EtOAc and filtered. The filtrate was washed with water and brine, dried over anhydrous Na₂SO₄, filtered and concentrated under vacuum. The residue was purified by column chromatography on silica gel (eluted with Petroleum Ether:EtOAc=10:1 to 4:1) to give compound A99-5 (60 mg, 0.17 mmol, 72.8% yield) as a light yellow solid. LC/MS (ESI) m/z: 517 (M+H)⁺.

### Step 5: Synthesis of Compound A99-6

10% Pd/C (60 mg) was added at room temperature to a solution of A99-5 (60 mg, 0.17 mmol) in THF, and the resulting mixture was degassed under N₂ atmosphere for three times and stirred under 15 psi H₂ at room temperature for 30 minutes. The mixture was then filtered and the filtrate was concentrated under vacuum. The residue was purified by column chromatography on silica gel (eluted with Petroleum Ether:EtOAc=10:1 to 3:1) to give compound A99-6 (40 mg, 0.09 mmol, 80.7% yield) as a white solid. LC/MS (ESI) m/z: 427 (M+H)⁺.

### Step 6: Synthesis of Compound A99

TFA (2 mL) was added dropwise at 0° C. to a solution of A99-6 (40 mg, 0.09 mmol) in DCM (2 mL) and the reaction was stirred at room temperature for 1 hour. The mixture was then concentrated under vacuum and the residue was solubilized in saturated aq. NaHCO₃ solution and extracted with EtOAc twice. The combined organic layers were washed with brine, dried over anhydrous Na₂SO₄, filtered and concentrated under vacuum. The residue was purified by prep-HPLC (C18, 0% to 50% acetonitrile in H₂O with 0.1% NH₃.H₂O) to give compound A99 (24 mg, 0.07 mmol, 78.4% yield) as a white solid. LC-MS: m/z 327 (M+H)⁺. ¹H NMR (400 MHz, DMSO-d6) δ 9.14 (s, 1H), 8.56 (br s, 1H), 7.63 (d, J=9.4 Hz, 1H), 7.38-7.30 (m, 3H), 7.16 (d, J=7.0 Hz, 2H), 6.90 (d, J=8.9 Hz, 1H), 4.56-4.51 (m, 1H), 4.47-4.40 (m, 1H), 3.53-3.46 (m, 1H), 3.44-3.38 (m, 1H).

The compounds in the table below were prepared from the appropriate starting materials described previously or commercially available using the above general procedure 5.

| Structure | Name |
|---|---|
| 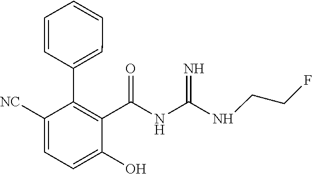 [SMILES: N#Cc1ccc(O)c(C(=O)NC(=N)NCCF)c1-c1ccccc1] | 6-cyano-N-(N-(2-fluoroethyl)carbamimidoyl)-3-hydroxy- [1,1′-biphenyl]-2-carboxamide LC-MS: m/z 327 (M + H)⁺. 1H NMR (400 MHz, DMSO) δ 9.14 (s, 1H), 8.56 (br s, 1H), 7.63 (d, J = 9.4 Hz, 1H), 7.38-7.30 (m, 3H), 7.16 (d, J = 7.0 Hz, 2H), 6.90 (d, J = 8.9 Hz, 1H), 4.56-4.51 (m, 1H), 4.47-4.40 (m, 1H), 3.53-3.46 (m, 1H), 3.44-3.38 (m, 1H). |
| 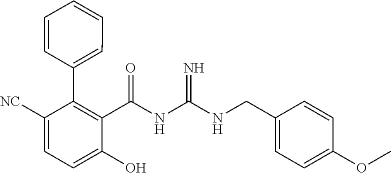 [SMILES: COc1ccc(CNC(=N)NC(=O)c2c(O)ccc(C#N)c2-c2ccccc2)cc1] | 6-cyano-3-hydroxy-N-(N-(4- methoxybenzyl)carbamimidoyl)-[1,1′-biphenyl]-2- carboxamide LC-MS: m/z 401 (M + H)⁺. 1H NMR (400 MHz, DMSO) δ 9.27 (br s, 1H), 8.47 (br s, 1H), 7.62 (d, J = 7.8 Hz, 1H), 7.36-7.23 (m, 3H), 7.22 (d, J = 6.9 Hz, 2H), 7.15 (d, J = 7.1 Hz, 2H), 6.89 (t, J = 8.7 Hz, 3H), 4.30 (s, 2H), 3.73 (s, 3H). |
| 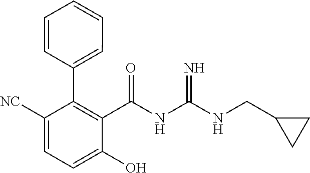 [SMILES: N#Cc1ccc(O)c(C(=O)NC(=N)NCC2CC2)c1-c1ccccc1] | 6-cyano-N-(N-(cyclopropylmethyl)carbamimidoyl)-3- hydroxy-[1,1′-biphenyl]-2-carboxamide LC-MS: m/z 335 (M + H)⁺. 1H NMR (400 MHz, DMSO) δ 7.61 (d, J = 8.7 Hz, 1H), 7.39-7.27 (m, 3H), 7.16 (d, J = 7.6 Hz, 2H), 6.87 (d, J = 8.7 Hz, 1H), 3.02 (d, J = 6.9 Hz, 2H), 1.04-0.96 (m, 1H), 0.48-0.42 (m, 2H), 0.22-0.16 (m, 2H). |
| 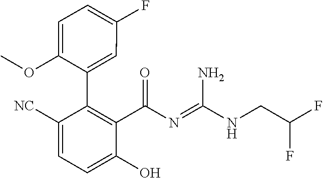 [SMILES: COc1ccc(F)cc1-c1c(C#N)ccc(O)c1C(=O)/N=C(\N)NCC(F)F] | N-(amino((2,2-difluoroethyl)amino)methylene)-6-cyano-5′- fluoro-3-hydroxy-2′-methoxy-[1,1′-biphenyl]-2- carboxamide LC-MS: m/z 393 (M + H)⁺. 1H NMR (400 MHz, DMSO) δ 8.71 (br s, 2H), 7.66 (d, J = 7.4 Hz, 1H), 7.11 (td, J = 8.7, 3.1 Hz, 1H), 7.01 (dd, J = 9.1, 4.6 Hz, 1H), 6.90 (dd, J = 8.8, 3.1 Hz, 2H), 6.12 (t, J = 55.3 Hz, 1H), 3.66-3.52 (m, 2H), 3.64 (s, 3H). |
| 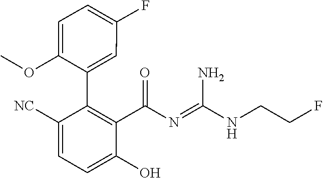 [SMILES: COc1ccc(F)cc1-c1c(C#N)ccc(O)c1C(=O)/N=C(\N)NCCF] | N-(amino((2-fluoroethyl)amino)methylene)-6-cyano-5′- fluoro-3-hydroxy-2′-methoxy-[1,1′-biphenyl]-2- carboxamide LC-MS: m/z 375 (M + H)⁺. 1H NMR (400 MHz, DMSO) δ 9.18 (br s, 1H), 8.62 (br s, 1H), 7.64 (d, J = 8.1 Hz, 1H), 7.11 (td, J = 8.7, 3.1 Hz, 1H), 7.01 (dd, J = 9.1, 4.6 Hz, 1H), 6.94-6.81 (m, 2H), 4.51 (d, J = 47.5 Hz, 2H), 3.65 (s, 3H), 3.53 (t, J = 4.8 Hz, 1H), 3.45 (dd, J = 14.8, 10.0 Hz, 1H). |
| 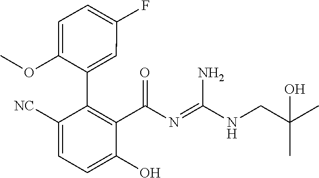 [SMILES: COc1ccc(F)cc1-c1c(C#N)ccc(O)c1C(=O)/N=C(\N)NCC(C)(C)O] | N-(amino((2-hydroxy-2-methylpropyl)amino)methylene)-6- cyano-5′-fluoro-3-hydroxy-2′-methoxy-[1,1′-biphenyl]-2- carboxamide LC-MS: m/z 401 (M + H)⁺. 1H NMR (400 MHz, DMSO) δ 9.31 (br s, 1H), 7.60 (d, J = 8.6 Hz, 1H), 7.10 (td, J = 8.7, 3.1 Hz, 1H), 7.00 (dd, J = 9.0, 4.5 Hz, 1H), 6.87 (t, J = 10.8 Hz, 1H), 4.77 (s, 1H), 3.64 (s, 3H), 3.10 (s, 2H), 1.07 (s, 6H). |
| 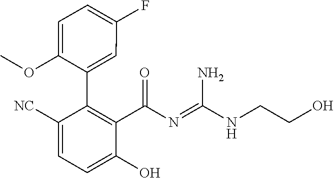 [SMILES: COc1ccc(F)cc1-c1c(C#N)ccc(O)c1C(=O)/N=C(\N)NCCO] | N-(amino((2-hydroxyethyl)amino)methylene)-6-cyano-5′- fluoro-3-hydroxy-2′-methoxy-[1,1′-biphenyl]-2- carboxamide LC-MS: m/z 373 (M + H)⁺. 1H NMR (400 MHz, DMSO) δ 9.13 (br s, 1H), 8.56 (br s, 1H), 7.60 (d, J = 8.9 Hz, 1H), 7.10 (td, J = 8.7, 3.1 Hz, 1H), 7.00 (dd, J = 9.1, 4.6 Hz, 1H), 6.85 (d, J = 7.6 Hz, 2H), 4.93 (s, 1H), 3.64 (s, 3H), 3.51-3.43 (m, 2H), 3.25-3.18 (m, 2H). |
| 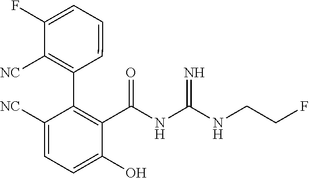 [SMILES: N#Cc1ccc(O)c(C(=O)NC(=N)NCCF)c1-c1cccc(F)c1C#N] | 2′,6-dicyano-3′-fluoro-N-(N-(2- fluoroethyl)carbamimidoyl)-3-hydroxy-[1,1′-biphenyl]-2- carboxamide LC-MS: m/z 370 (M + H)⁺. 1H NMR (400 MHz, DMSO) δ 9.18 (s, 1H), 8.61 (s, 1H), 7.85-7.75 (m, 2H), 7.52 (t, J = 8.9 Hz, 1H), 7.27 (d, J = 7.8 Hz, 1H), 7.01 (d, J = 8.7 Hz, 1H), 4.50 (d, J = 46.9 Hz, 2H), 3.52 (d, J = 27.6 Hz, 2H). |
| 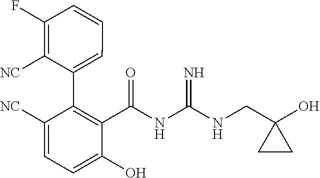 [SMILES: N#Cc1ccc(O)c(C(=O)NC(=N)NCC2(O)CC2)c1-c1cccc(F)c1C#N] | 2′,6-dicyano-3′-fluoro-3-hydroxy-N-(N-((1- hydroxycyclopropyl)methyl)carbamimidoyl)-[1,1′- biphenyl]-2-carboxamide LC-MS: m/z 394 (M + H)⁺. 1H NMR (400 MHz, DMSO) δ: 9.22 (s, 1H), 8.52 (s, 1H), 7.83-7.72 (m, 2H), 7.52 (t, J = 8.8 Hz, 1H), 7.27 (d, J = 7.7 Hz, 1H), 6.99 (d, J = 8.8 Hz, 1H), 5.59 (s, 1H), 3.28 (s, 2H), 0.66-0.52 (m, 4H). |
| 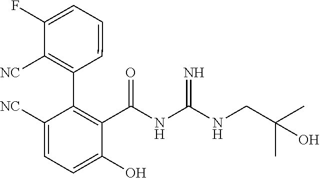 [SMILES: CC(C)(O)CNC(=N)NC(=O)c1c(O)ccc(C#N)c1-c1cccc(F)c1C#N] | 2′,6-dicyano-3′-fluoro-3-hydroxy-N-(N-(2-hydroxy-2- methylpropyl)carbamimidoyl)biphenyl-2-carboxamide LC-MS: m/z 396 (M + H)⁺. 1H NMR (400 MHz, DMSO) δ 9.27 (s, 1H), 8.49 (s, 1H), 7.84-7.71 (m, 2H), 7.52 (t, J = 8.8 Hz, 1H), 7.27 (d, J = 7.6 Hz, 1H), 7.11-6.89 (m, 1H), 4.80 (s, 1H), 3.18-3.09 (m, 2H), 1.12 (s, 6H). |
| 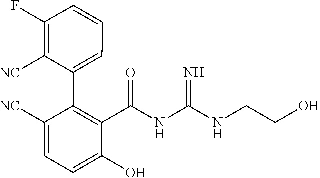 [SMILES: N#Cc1ccc(O)c(C(=O)NC(=N)NCCO)c1-c1cccc(F)c1C#N] | 2′,6-dicyano-3′-fluoro-3-hydroxy-N-(N-(2- hydroxyethyl)carbamimidoyl)-[1,1′-biphenyl]-2- carboxamide LC-MS: m/z 368 (M + H)⁺. 1H NMR (400 MHz, DMSO) δ 7.82-7.71 (m, 2H), 7.51 (t, J = 9.0 Hz, 1H), 7.25 (d, J = 7.7 Hz, 1H), 6.96 (d, J = 8.8 Hz, 1H), 3.51 (t, J = 4.6 Hz, 2H), 3.24 (t, J = 5.0 Hz, 2H). |
| 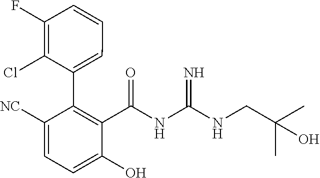 [SMILES: CC(C)(O)CNC(=N)NC(=O)c1c(O)ccc(C#N)c1-c1cccc(F)c1Cl] | 2′-chloro-6-cyano-3′-fluoro-3-hydroxy-N-(N-(2-hydroxy-2- methylpropyl)carbamimidoyl)-[1,1′-biphenyl]-2- carboxamide LC-MS: m/z 405 (M + H)⁺. 1H NMR (400 MHz, DMSO) δ 9.27 (s, 1H), 7.68 (d, J = 8.8 Hz, 1H), 7.37 (m, 2H), 7.07 (d, J = 6.2 Hz, 1H), 6.92 (d, J = 8.5 Hz, 1H), 4.79 (s, 1H), 3.11 (s, 2H), 1.11 (s, 6H). |
| 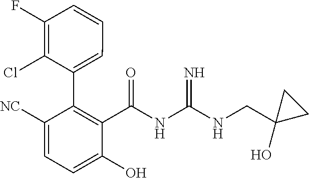 [SMILES: N#Cc1ccc(O)c(C(=O)NC(=N)NCC2(O)CC2)c1-c1cccc(F)c1Cl] | 2′-chloro-6-cyano-3′-fluoro-3-hydroxy-N-(N-((1- hydroxycyclopropyl)methyl)carbamimidoyl)-[1,1′- biphenyl]-2-carboxamide LC-MS: m/z 403 (M + H)⁺. 1H NMR (400 MHz, DMSO) δ 9.22 (s, 1H), 7.69 (d, J = 8.7 Hz, 1H), 7.41-7.34 (m, 2H), 7.11-7.02 (m, 1H), 6.93 (d, J = 9.0 Hz, 1H), 5.59 (s, 1H), 3.27 (s, 2H), 0.64-0.51 (m, 4H). |
| 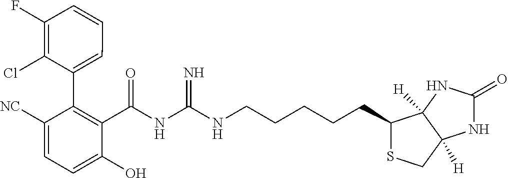 [SMILES: N#Cc1ccc(O)c(C(=O)NC(=N)NCCCCC[C@@H]2SC[C@@H]3NC(=O)N[C@@H]32)c1-c1cccc(F)c1Cl] | 2′-chloro-6-cyano-3′-fluoro-3-hydroxy-N-(N-(5- ((3aS,4S,6aR)-2-oxohexahydro-1H-thieno[3,4-d]imidazol- 4-yl)pentyl)carbamimidoyl)-[1,1′-biphenyl]-2-carboxamide LC-MS: m/z 545 (M + H)⁺. 1H NMR (400 MHz, DMSO) δ 8.90 (s, 1H), 8.09 (br s, 1H), 7.68 (d, J = 8.3 Hz, 1H), 7.41-7.32 (m, 2H), 7.09-7.02 (m, 1H), 6.94 (d, J = 8.9 Hz, 1H), 6.30 (s, 1H), 6.26 (s, 1H), 4.36-4.28 (m, 1H), 4.19-4.12 (m, 1H), 3.19-3.09 (m, 3H), 2.83 (dd, J = 12.4, 5.1 Hz, 1H), 2.60 (d, J = 12.4 Hz, 1H), 1.65-1.28 (m, 8H). |
| 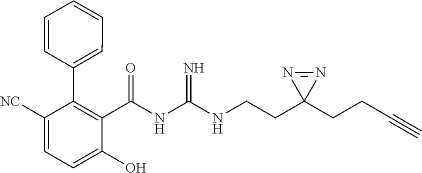 [SMILES: C#CCCC1(CCNC(=N)NC(=O)c2c(O)ccc(C#N)c2-c2ccccc2)N=N1] | N-(N-(2-(3-(but-3-yn-1-yl)-3H-diazirin-3- yl)ethyl)carbamimidoyl)-6-cyano-3-hydroxy-[1,1′- biphenyl]-2-carboxamide LC-MS: m/z 401 (M + H)⁺. 1H NMR (400 MHz, DMSO) δ 8.88 (s, 1H), 8.42 (br s, 1H), 7.62 (d, J = 8.3 Hz, 1H), 7.42-7.27 (m, 3H), 7.16 (d, J = 6.5 Hz, 2H), 6.88 (d, J = 8.4 Hz, 1H), 3.08-2.94 (m, 2H), 2.81 (s, 1H), 2.07-1.90 (m, 2H), 1.71-1.52 (m, 4H). |
| 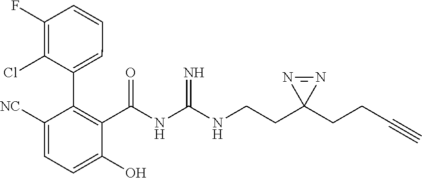 [SMILES: C#CCCC1(CCNC(=N)NC(=O)c2c(O)ccc(C#N)c2-c2cccc(F)c2Cl)N=N1] | N-(N-(2-(3-(but-3-yn-1-yl)-3H-diazirin-3- yl)ethyl)carbamimidoyl)-2′-chloro-6-cyano-3′-fluoro-3- hydroxy-[1,1′-biphenyl]-2-carboxamide LC-MS: m/z 453 (M + H)⁺. 1H NMR (400 MHz, DMSO) δ 8.53 (br s, 1H), 7.69 (d, J = 8.9 Hz, 1H), 7.42-7.32 (m, 2H), 7.06 (d, J = 5.8 Hz, 1H), 6.92 (d, J = 8.6 Hz, 1H), 3.11-2.96 (m, 2H), 2.81 (s, 1H), 2.04-1.90 (m, 2H), 1.75-1.48 (m, 4H). |
| 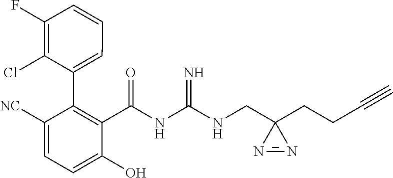 [SMILES: C#CCCC1(CNC(=N)NC(=O)c2c(O)ccc(C#N)c2-c2cccc(F)c2Cl)N=N1] | N-(N-((3-(but-3-yn-1-yl)-3H-diazirin-3- yl)methyl)carbamimidoyl)-2′-chloro-6-cyano-3′-fluoro-3- hydroxy-[1,1′-biphenyl]-2-carboxamide LC-MS: m/z 439 (M + H)⁺. 1H NMR (400 MHz, DMSO) δ 8.67 (br, 1H), 7.73 (s, 1H), 7.43-7.33 (m, 2H), 7.01 (m, 2H), 3.30 (m, 2H), 2.81 (s, 1H), 1.99 (m, 2H), 1.66 (m, 2H). |
| 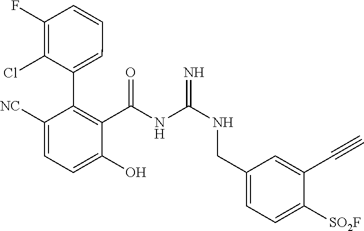 [SMILES: C#Cc1cc(CNC(=N)NC(=O)c2c(O)ccc(C#N)c2-c2cccc(F)c2Cl)ccc1S(=O)(=O)F] | 4-((3-(2′-chloro-6-cyano-3′-fluoro-3-hydroxy-[1,1′- biphenyl]-2-carbonyl)guanidino)methyl)-2- ethynylbenzenesulfonyl fluoride LC-MS: m/z 529 (M + H)⁺. 1H NMR (400 MHz, DMSO) δ 9.45 (s, 1H), 8.18 (d, J = 8.3 Hz, 1H), 7.78 (d, J = 1.2 Hz, 1H), 7.74 (d, J = 8.7 Hz, 1H), 7.62 (d, J = 4.9 Hz, 1H), 7.40-7.35 (m, 2H), 7.08 (d, J = 5.0 Hz, 1H), 6.97 (d, J = 8.2 Hz, 1H), 4.98 (s, 1H), 4.57 (d, J = 6.3 Hz, 2H). |
| 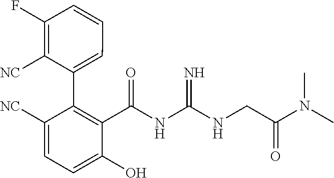 [SMILES: CN(C)C(=O)CNC(=N)NC(=O)c1c(O)ccc(C#N)c1-c1cccc(F)c1C#N] | 2′,6-dicyano-N-(N-(2-(dimethylamino)-2- oxoethyl)carbamimidoyl)-3′-fluoro-3-hydroxy-[1,1′- biphenyl]-2-carboxamide LC-MS: m/z 409 (M + H)⁺. 1H NMR (400 MHz, DMSO) δ 9.43 (s, 1H), 7.87 (s, 1H), 7.82-7.70 (m, 2H), 7.50 (dd, J = 8.8, 8.8 Hz, 1H), 7.25 (d, J = 7.2 Hz, 1H), 7.08-6.94 (m, 1H), 4.09 (s, 2H), 2.92 (s, 3H), 2.86 (s, 3H). |

### General Procedure 6

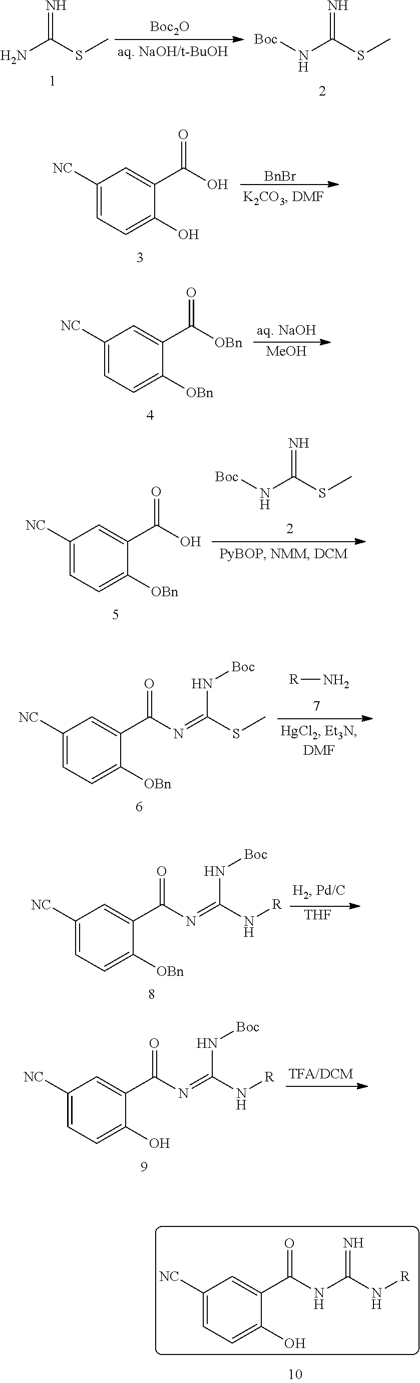

### Step 1: Synthesis of Compound 2

Methyl carbamimidothioate (9 g, 100 mmol) was dissolved in a solution of NaOH (4 g, 100 mmol) in water (10 mL) and t-BuOH (100 mL). A solution of Boc₂O (19.6 g, 90 mmol) in t-BuOH (50 mL) was then added dropwise at 0° C. over a period of 1 hour and the resulting mixture was stirred overnight at room temperature. The mixture was then diluted with water and extracted with DCM. The organic layer was washed with brine, dried over anhydrous Na₂SO₄, filtered and concentrated under vacuum. The residue was purified by column chromatography on silica gel (eluted with Petroleum Ether:EtOAc=100:0 to 5:1) to give compound 2 (8.1 g, 42.6% yield) as a white solid. LC/MS (ESI) m/z: 191 (M+H)⁺.

### Step 2: Synthesis of Compound 4

Benzyl bromide (8.55 g, 50 mmol) was added dropwise to a mixture of compound 3 (3.26 g, 20 mmol) and potassium carbonate (8.28 g, 60 mmol) in DMF (40 mL) and the resulting mixture was stirred at room temperature for 16 hours. After the reaction was completed, the mixture was diluted with EtOAc and washed with water. The organic layer was then dried over Na₂SO₄, filtered and concentrated under vacuum. The residue was purified by column chromatography on silica gel (eluted with Petroleum Ether:EtOAc=100:0 to 20:1) to give compound 4 (3.3 g, 48.1% yield) as a colorless oil. LC/MS (ESI) m/z: 366 (M+Na)⁺.

### Step 3: Synthesis of Compound 5

A solution of NaOH (962 mg, 24 mmol) in water (10 mL) was added to a solution of compound 4 (3.3 g, 9.62 mmol) in MeOH (20 mL) and the resulting mixture was stirred at room temperature for 16 hours. The mixture was then diluted with water and extracted with Et₂O. The aqueous layer was separated and acidified with 1 N HCl solution to pH=5. Then the mixture was extracted with EtOAc twice and the combined organic layers were dried over anhydrous Na₂SO₄, filtered and concentrated under vacuum. The residue was purified by column chromatography on silica gel (eluted with DCM:MeOH=100:0 to 40:1) to give compound 5 (2.1 g, 86.3% yield) as a colorless oil. LC/MS (ESI) m/z: 252 (M−H)⁻.

The synthesis from 6 to 10 is the same as in the general procedure 5 described above.

### Synthesis of F28

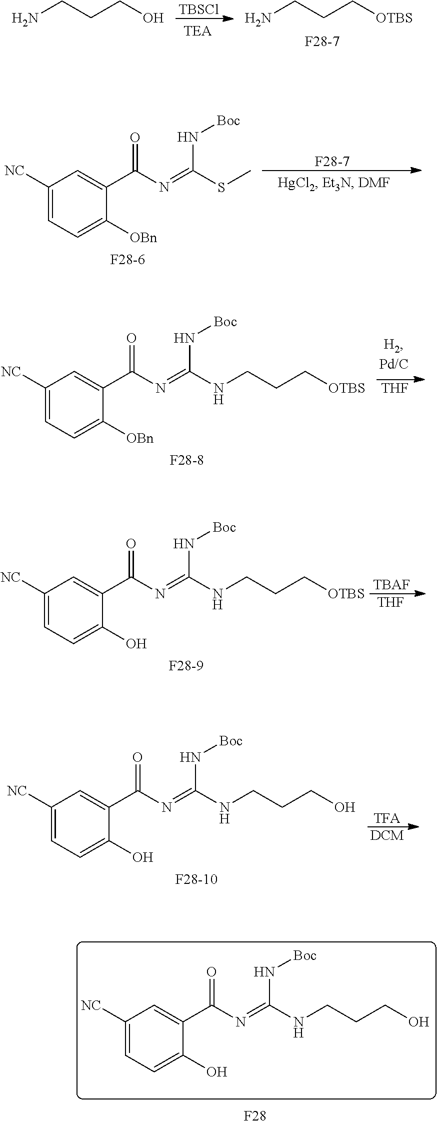

### Step 1: Synthesis of Compound F28-7

TBSCl (1.1 g, 7.3 mmol) was added at 0° C. to a solution of 1-amino-3-propanol (0.5 g, 6.6 mmol) and Et₃N (1.4 mL, 9.9 mmol) in DCM (15 mL), and the resulting mixture was stirred at room temperature for 12 hours. The mixture was then washed with water and the organic layer was separated, dried over MgSO₄, filtered and concentrated under vacuum. The residue was purified by column chromatography on silica gel (eluted with DCM:MeOH=20:1 to 5:1) to give compound F28-7 as a yellow oil (1.2 g, 6.3 mmol, 95% yield).

The synthesis from F28-6 to F28 is the same as in the general procedure 5 described above.

The compounds in the table below were prepared from the appropriate starting materials described previously or commercially available using the above general procedure 6.

| Structure | Name |
|---|---|
| 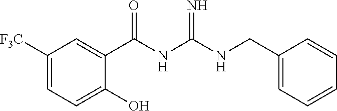 [SMILES: N=C(NCc1ccccc1)NC(=O)c1cc(C(F)(F)F)ccc1O] | N-(N-benzylcarbamimidoyl)-2-hydroxy-5-(trifluoromethyl)benzamide LC-MS: m/z 338 (M + H)⁺. 1H NMR (400 MHz, DMSO-d6) δ 15.49 (s, 1H), 9.67 (s, 1H), 8.82 (s, 1H), 8.11 (s, 1H), 7.82 (s, 1H), 7.61 (d, J = 8.5 Hz, 1H), 7.45-7.21 (m, 5H), 6.95 (d, J = 8.6 Hz, 1H), 4.47 (s, 2H). |
| 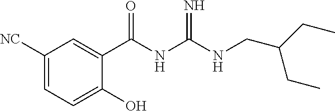 [SMILES: CCC(CC)CNC(=N)NC(=O)c1cc(C#N)ccc1O] | 5-cyano-N-(N-(2-ethylbutyl)carbamimidoyl)-2-hydroxybenzamide LC-MS: m/z 289 (M + H)⁺. 1H NMR (400 MHz, DMSO-d6) δ 16.17 (s, 1H), 9.46 (s, 1H), 8.60 (s, 1H), 8.12 (s, 1H), 7.66 (d, J = 8.7 Hz, 1H), 6.87 (d, J = 8.6 Hz, 1H), 3.15 (d, J = 8.0 Hz, 2H), 1.57-1.48 (m, 1H), 1.35 (dd, J = 14.5, 7.1 Hz, 4H), 0.87 (t, J = 7.4 Hz, 7H). |
| 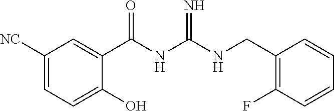 [SMILES: N#Cc1ccc(O)c(C(=O)NC(=N)NCc2ccccc2F)c1] | 5-cyano-N-(N-(2-fluorobenzyl)carbamimidoyl)-2-hydroxybenzamide LC-MS: m/z 313 (M + H)⁺. 1H NMR (400 MHz, DMSO-d6) δ 15.96 (s, 1H), 9.65 (s, 1H), 8.90 (br s, 1H), 8.13 (s, 1H), 7.68 (d, J = 7.9 Hz, 1H), 7.47-7.33 (m, 2H), 7.30-7.18 (m, 2H), 6.90 (d, J = 8.6 Hz, 1H), 4.53 (s, 2H). |
| 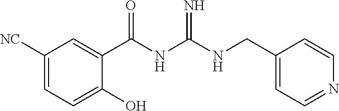 [SMILES: N#Cc1ccc(O)c(C(=O)NC(=N)NCc2ccncc2)c1] | 5-cyano-2-hydroxy-N-(N-(pyridin-4- ylmethyl)carbamimidoyl)benzamide LC-MS: m/z 296 (M + H)⁺. 1H NMR (400 MHz, DMSO-d6) δ 16.00 (s, 1H), 8.56 (d, J = 5.2 Hz, 2H), 8.14 (d, J = 2.0 Hz, 1H), 7.69 (dd, J = 8.6, 2.3 Hz, 1H), 7.32 (d, J = 5.8 Hz, 2H), 6.90 (d, J = 8.3 Hz, 1H), 4.53 (s, 2H). |
| 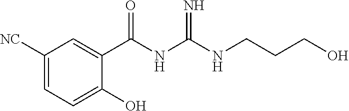 [SMILES: N#Cc1ccc(O)c(C(=O)NC(=N)NCCCO)c1] | 5-cyano-2-hydroxy-N-(N-(3-hydroxypropyl)carbamimidoyl)benzamide LC-MS: m/z 263 (M + H)⁺. 1H NMR (400 MHz, DMSO-d6) δ 16.22 (s, 1H), 9.29 (s, 1H), 8.11 (s, 1H), 7.65 (d, J = 8.4 Hz, 1H), 6.86 (d, J = 8.6 Hz, 1H), 4.64 (d, J = 5.2 Hz, 1H), 3.52-3.46 (m, 3H), 3.29-3.23 (m, 3H), 1.74-1.66 (m, 2H). |
| 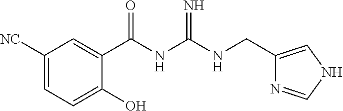 [SMILES: N#Cc1ccc(O)c(C(=O)NC(=N)NCc2c[nH]cn2)c1] | N-(N-((1H-imidazol-4-yl)methyl)carbamimidoyl)-5-cyano-2- hydroxybenzamide LC-MS: m/z 285 (M + H)⁺. 1H NMR (400 MHz, DMSO-d6) δ 16.07 (s, 1H), 9.15 (br s, 2H), 8.10 (d, J = 1.7 Hz, 1H), 7.69-7.61 (m, 2H), 7.09 (s, 1H), 6.86 (d, J = 8.6 Hz, 1H), 4.32 (s, 2H). |
| 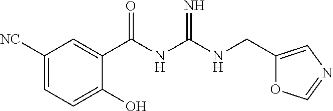 [SMILES: N#Cc1ccc(O)c(C(=O)NC(=N)NCc2cnco2)c1] | 5-cyano-2-hydroxy-N-(N-(oxazol-5- ylmethyl)carbamimidoyl)benzamide LC-MS: m/z 286 (M + H)⁺. 1H NMR (400 MHz, DMSO-d6) δ 15.81 (s, 1H), 8.66 (br s, 2H), 8.37 (s, 1H), 8.14 (s, 1H), 7.68 (d, J = 8.2 Hz, 1H), 7.14 (s, 1H), 6.90 (d, J = 8.5 Hz, 1H), 4.56 (s, 2H). |

### General Procedure 7

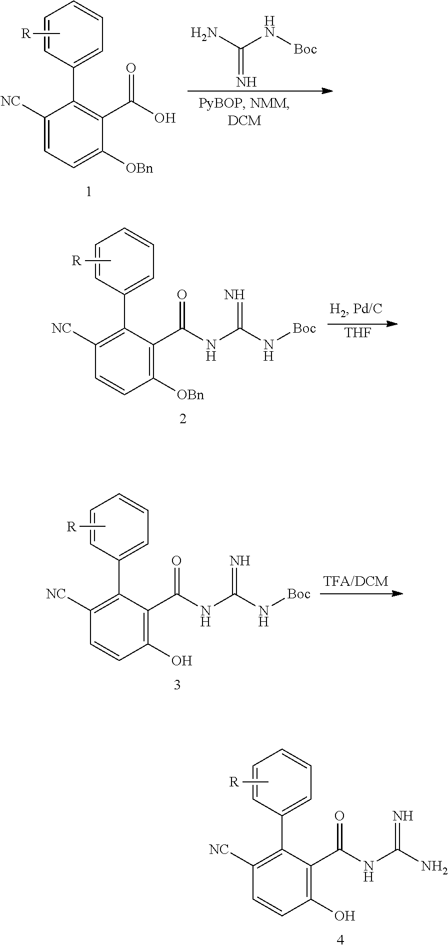

### Step 1: Synthesis of Compound 1

The synthesis of compound 1 was described in general procedure 5.

### Step 2: Synthesis of Compound 2

tert-butyl N-carbamimidoylcarbamate (2.6 eq.) and PyBOP (1.4 eq.) were added under N₂ atmosphere to a mixture of 1 (1 eq.) and NMM (4 eq.) in DMF. The resulting mixture was stirred at room temperature for 15 hours. The mixture was then poured into water and extracted with EtOAc twice. The combined organic layers were washed with brine, dried over Na₂SO₄, filtered and concentrated under vacuum. The residue was purified by flash column chromatography on silica gel (eluted with Petroleum Ether:EA=10:1 to 3:1) to give compound 2.

### Step 4: Synthesis of Compound 3

10% Pd/C (1:1, w/w) was added under N₂ atmosphere to a solution of 2 (1 eq.) in THF and the resulting mixture was stirred at room temperature under H₂ atmosphere for 1 hour. The mixture was then filtered and the filtrate was concentrated under vacuum to give compound 3.

### Step 5: Synthesis of Compound 4

TFA (1:1, v/v) was added dropwise at 0° C. to a solution of 3 (1 eq.) in DCM and the reaction mixture was slowly warmed to room temperature and stirred for 2 hours. The solvent was then removed under vacuum and the crude was purified by prep-HPLC (C18, 0% to 50% acetonitrile in H₂O with 0.1% NH₃.H₂O) to give compound 4.

### Synthesis of A39

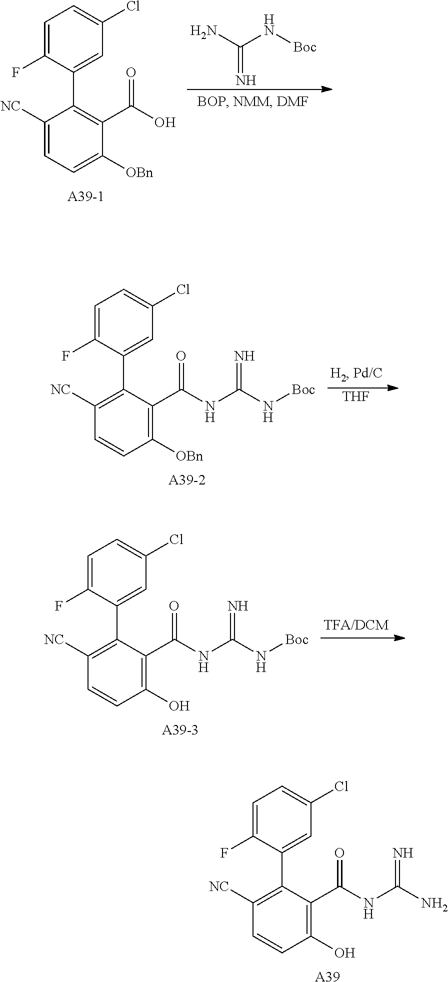

### Step 1: Synthesis of Compound A39-2

tert-butyl N-carbamimidoylcarbamate (91.0 mg, 0.572 mmol) was added under N₂ atmosphere to a solution of A39-1 (84 mg, 0.22 mmol) and NMM (88.9 mg, 0.88 mmol) in DMF (6 mL) followed by PyBOP (136.2 mg, 0.31 mmol). The resulting mixture was stirred at room temperature for 15 hours. The mixture was then poured into water and extracted with EtOAc twice. The combined organic layers were washed with brine, dried over Na₂SO₄, filtered and concentrated under vacuum. The residue was purified by flash column chromatography on silica gel (eluted with Petroleum Ether:EA=10:1 to 3:1) to give A39-2 (100 mg, 86.92% yield) as a white solid. LC/MS (ESI) m/z: 523 (M+H)⁺.

### Step 2: Synthesis of Compound A39-3

10% Pd/C (100 mg) was added under N₂ atmosphere to a solution of A39-2 (100 mg, 0.19 mmol) in THF (5 mL) and the mixture was stirred at room temperature under 15 psi H₂ for 1 hour. The mixture was then filtered and the filtrate was concentrated to give A39-2 (80 mg, 96.7% yield) as a white solid without any further purification. LC/MS (ESI) m/z: 433 (M+H)⁺.

### Step 3: Synthesis of Compound A39

TFA (2 mL) was added at 0° C. dropwise to a solution of A39-3 (80 mg, 0.185 mmol) in DCM (2 mL) and the mixture was slowly warmed to room temperature and stirred for 1 hour. The solvent was then removed under vacuum and the residue was purified via prep-HPLC (C18, 0% to 50% acetonitrile in H₂O with 0.1% NH₃.H₂O) to give A39 (18 mg, 29.27% yield) as a white solid. LC/MS (ESI) m/z: 333 (M+H)⁺, 1H NMR (400 MHz, DMSO) δ: 7.66 (d, J=8.8 Hz, 1H), 7.43 (ddd, J=8.7, 4.4, 2.7 Hz, 1H), 7.32 (dd, J=6.3, 2.7 Hz, 1H), 7.26 (t, J=9.0 Hz, 1H), 6.90 (d, J=8.8 Hz, 1H).

The compounds in the table below were prepared from the appropriate starting materials described previously or commercially available using the above general procedure 7

| Structure | Name |
|---|---|
| 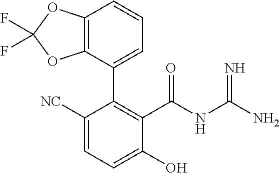 [SMILES: N#Cc1ccc(O)c(C(=O)NC(=N)N)c1-c1cccc2c1OC(F)(F)O2] | N-carbamimidoyl-3-cyano-2-(2,2- difluorobenzo[d][1,3]dioxol-4-yl)-6- hydroxybenzamide LC-MS: m/z 361 (M + H)⁺. 1H NMR (400 MHz, DMSO) δ 7.67 (d, J = 8.8 Hz, 1H), 7.35 (dd, J = 8.1, 1.0 Hz, 1H), 7.20 (t, J = 8.0 Hz, 1H), 7.04 (dd, J = 8.0, 1.0 Hz, 1H), 6.91 (d, J = 8.8 Hz, 1H). |
| 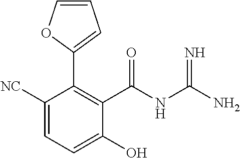 [SMILES: N#Cc1ccc(O)c(C(=O)NC(=N)N)c1-c1ccco1] | N-carbamimidoyl-3-cyano-2-(furan-2-yl)-6- hydroxybenzamide LC-MS: m/z 271 (M + H)⁺. 1H NMR (400 MHz, DMSO) δ 7.68 (s, 1H), 7.61 (d, J = 8.8 Hz, 1H), 6.87 (d, J = 8.7 Hz, 1H), 6.55-6.50 (m, 1H), 6.47 (d, J = 3.2 Hz, 1H). |
| 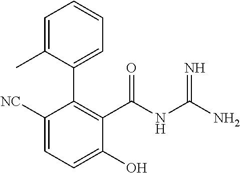 [SMILES: Cc1ccccc1-c1c(C#N)ccc(O)c1C(=O)NC(=N)N] | N-carbamimidoyl-6-cyano-3-hydroxy-2′-methyl- [1,1′-biphenyl]-2-carboxamide LC-MS: m/z 295 (M + H)⁺. 1H NMR (400 MHz, DMSO) δ 8.25 (br s, 2H), 7.62 (d, J = 8.7 Hz, 1H), 7.20-7.13 (m, 3H), 6.93 (d, J = 7.2 Hz, 1H), 6.85 (d, J = 8.7 Hz, 1H), 1.97 (s, 3H). |
| 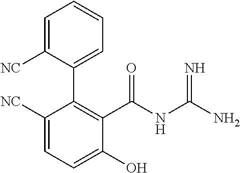 [SMILES: N#Cc1ccccc1-c1c(C#N)ccc(O)c1C(=O)NC(=N)N] | N-carbamimidoyl-2′,6-dicyano-3-hydroxy-[1,1′- biphenyl]-2-carboxamide LC-MS: m/z 306 (M + H)⁺. 1H NMR (400 MHz, DMSO) δ 7.85 (d, J = 7.6 Hz, 1H), 7.81 (br s, 2H), 7.78-7.62 (m, 2H), 7.51 (t, J = 8.0 Hz, 1H), 7.36 (d, J = 7.6 Hz, 1H), 6.91 (d, J = 8.8 Hz, 1H). |
| 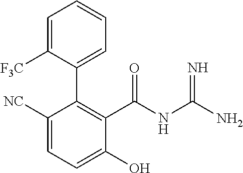 [SMILES: N#Cc1ccc(O)c(C(=O)NC(=N)N)c1-c1ccccc1C(F)(F)F] | N-carbamimidoyl-6-cyano-3-hydroxy-2′- (trifluoromethyl)-[1,1′-biphenyl]-2-carboxamide LC-MS: m/z 349 (M + H)⁺. 1H NMR (400 MHz, DMSO) δ 8.24 (br s, 2H), 7.73 (d, J = 8.0 Hz, 1H), 7.68-7.58 (m, 2H), 7.53 (t, J = 7.6 Hz, 1H), 7.24 (d, J = 7.6 Hz, 1H), 7.22 (br s, 1H), 6.88 (d, J = 8.8 Hz, 1H). |
| 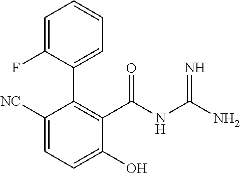 [SMILES: N#Cc1ccc(O)c(C(=O)NC(=N)N)c1-c1ccccc1F] | N-carbamimidoyl-6-cyano-2′-fluoro-3-hydroxy- [1,1′-biphenyl]-2-carboxamide LC-MS: m/z 299 (M + H)⁺. 1H NMR (400 MHz, DMSO) δ 7.74 (br s, 2H), 7.63 (d, J = 8.8 Hz, 1H), 7.41-7.32 (m, 1H), 7.25-7.12 (m, 3H), 6.87 (d, J = 8.4 Hz, 1H). |
| 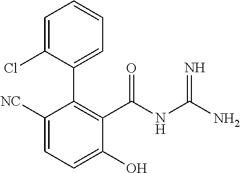 [SMILES: N#Cc1ccc(O)c(C(=O)NC(=N)N)c1-c1ccccc1Cl] | N-carbamimidoyl-2′-chloro-6-cyano-3-hydroxy- [1,1′-biphenyl]-2-carboxamide LC-MS: m/z 315 (M + H)⁺. NMR (400 MHz, DMSO) δ 7.74 (br s, 2H), 7.63 (d, J = 8.7 Hz, 1H), 7.47-7.40 (m, 1H), 7.33 (dd, J = 5.7, 3.4 Hz, 2H), 7.22-7.15 (m, 1H), 6.87 (d, J = 8.7 Hz, 1H). |
| 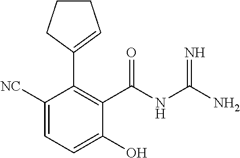 [SMILES: N#Cc1ccc(O)c(C(=O)NC(=N)N)c1C1=CCCC1] | N-carbamimidoyl-3-cyano-2-(cyclopent-1-en-1- yl)-6-hydroxybenzamide LC-MS: m/z 271 (M + H)⁺. 1H NMR (400 MHz, DMSO) δ 7.85 (br s, 3H), 7.53 (d, J = 8.8 Hz, 1H), 6.74 (d, J = 8.8 Hz, 1H), 5.44 (s, 1H), 2.60-2.52 (m, 2H), 2.48-2.38 (m, 2H), 2.05-1.93 (m, 2H). |
| 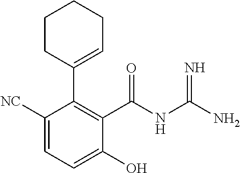 [SMILES: N#Cc1ccc(O)c(C(=O)NC(=N)N)c1C1=CCCCC1] | N-carbamimidoyl-6-cyano-3-hydroxy-2′,3′,4′,5′- tetrahydro-[1,1′-biphenyl]-2-carboxamide LC-MS: m/z 285 (M + H)⁺. 1H NMR (400 MHz, DMSO) δ 8.27 (br s, 2H), 7.51 (d, J = 8.4 Hz, 1H), 7.32 (br s, 1H), 6.72 (d, J = 8.4 Hz, 1H), 5.31 (s, 1H), 2.38-1.86 (m, 4H), 1.80-1.51 (m, 4H). |
| 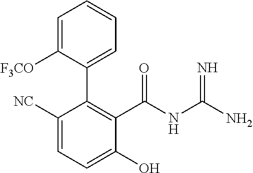 [SMILES: N#Cc1ccc(O)c(C(=O)NC(=N)N)c1-c1ccccc1OC(F)(F)F] | N-carbamimidoyl-6-cyano-3-hydroxy-2′- (trifluoromethoxy)-[1,1′-biphenyl]-2- carboxamide LC-MS: m/z 365 (M + H)⁺. 1H NMR (400 MHz, DMSO) δ 7.77 (br s, 2H), 7.65 (d, J = 8.8 Hz, 1H), 7.46 (t, J = 7.6 Hz, 1H), 7.43-7.33 (m, 2H), 7.30 (d, J = 7.6 Hz, 1H), 6.89 (d, J = 8.8 Hz, 1H). |
| 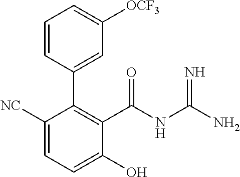 [SMILES: N#Cc1ccc(O)c(C(=O)NC(=N)N)c1-c1cccc(OC(F)(F)F)c1] | N-carbamimidoyl-6-cyano-3-hydroxy-3′- (trifluoromethoxy)-[1,1′-biphenyl]-2- carboxamide LC-MS: m/z 365 (M + H)⁺. 1H NMR (400 MHz, DMSO) δ 7.62 (d, J = 8.7 Hz, 1H), 7.47 (t, J = 7.9 Hz, 1H), 7.30 (d, J = 8.2 Hz, 1H), 7.19 (d, J = 7.6 Hz, 1H), 7.15 (s, 1H), 6.87 (d, J = 8.7 Hz, 1H). |
| 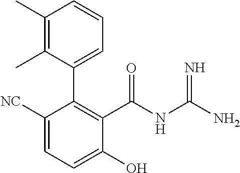 [SMILES: Cc1cccc(-c2c(C#N)ccc(O)c2C(=O)NC(=N)N)c1C] | N-carbamimidoyl-6-cyano-3-hydroxy-2′,3′- dimethyl-[1,1′-biphenyl]-2-carboxamide LC-MS: m/z 309 (M + H)⁺. 1H NMR (400 MHz, DMSO) δ 8.25 (br s, 2H), 7.60 (d, J = 8.8 Hz, 1H), 7.14 (br s, 1H), 7.09- 7.01 (m, 2H), 6.83 (d, J = 8.8 Hz, 1H), 6.76 (d, J = 7.2 Hz, 1H), 2.26 (s, 3H), 1.88 (s, 3H). |
| 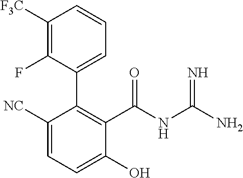 [SMILES: N#Cc1ccc(O)c(C(=O)NC(=N)N)c1-c1cccc(C(F)(F)F)c1F] | N-carbamimidoyl-6-cyano-2′-fluoro-3-hydroxy- 3′-(trifluoromethyl)-[1,1′-biphenyl]-2- carboxamide LC-MS: m/z 367 (M + H)⁺. 1H NMR (400 MHz, DMSO) δ 7.75 (t, J = 6.8 Hz, 1H), 7.69 (d, J = 8.8 Hz, 1H), 7.57 (t, J = 6.8 Hz, 1H), 7.41 (t, J = 8.0 Hz, 1H), 6.92 (d, J = 8.8 Hz, 1H). |
| 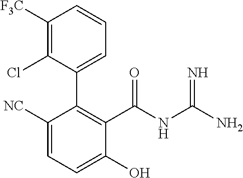 [SMILES: N#Cc1ccc(O)c(C(=O)NC(=N)N)c1-c1cccc(C(F)(F)F)c1Cl] | N-carbamimidoyl-2′-chloro-6-cyano-3-hydroxy- 3′-(trifluoromethyl)-[1,1′-biphenyl]-2- carboxainide LC-MS: m/z 383 (M+H)⁺. 1H NMR (400 MHz, DMSO) δ 7.82 (dd. J = 7.2, 1.9 Hz, 1H), 7.68 (d, J = 8.8 Hz, 1H), 7.54 (q, J = 7.7 Hz, 2H), 6.91 (d, J = 8.8 Hz, 1H). |
| 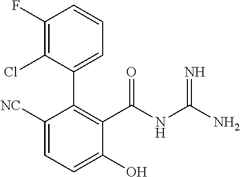 [SMILES: N#Cc1ccc(O)c(C(=O)NC(=N)N)c1-c1cccc(F)c1Cl] | N-carbamimidoyl-2′-chloro-6-cyano-3′-fluoro-3- hydroxy-[1,1′-biphenyl]-2-carboxamide LC-MS: m/z 333 (M + H)⁺. 1H NMR (400 MHz, DMSO) δ 8.26 (br s, 2H), 7.67 (d, J = 8.8 Hz, 1H), 7.43-7.28 (m, 2H), 7.22 (br s, 1H), 7.06 (d, J = 6.4 Hz, 1H), 6.90 (d, J = 8.4 Hz, 1H). |
| 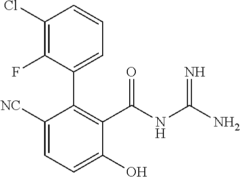 [SMILES: N#Cc1ccc(O)c(C(=O)NC(=N)N)c1-c1cccc(Cl)c1F] | N-carbamimidoyl-3′-chloro-6-cyano-2′-fluoro-3- hydroxy-[1,1′-biphenyl]-2-carboxamide LC-MS: m/z 333 (M + H)⁺. 1H NMR (400 MHz, DMSO) δ 8.08 (br s, 2H), 7.68 (d, J = 8.8 Hz, 1H), 7.55 (t, J = 7.2 Hz, 1H), 7.28-7.15 (m, 2H), 6.92 (d, J = 8.8 Hz, 1H). |
| 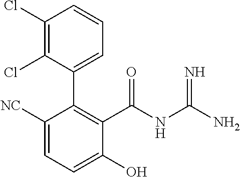 [SMILES: N#Cc1ccc(O)c(C(=O)NC(=N)N)c1-c1cccc(Cl)c1Cl] | N-carbamimidoyl-2′,3′-dichloro-6-cyano-3- hydroxy-[1,1′-biphenyl]-2-carboxamide LC-MS: m/z 349 (M + H)⁺. 1H NMR (400 MHz, DMSO) δ 7.66 (d, J = 8.7 Hz, 1H), 7.58 (d, J = 8.0 Hz, 1H), 7.36 (t, J = 7.9 Hz, 1H), 7.18 (d, J = 7.6 Hz, 1H), 6.89 (d, J = 8.7 Hz, 1H). |
| 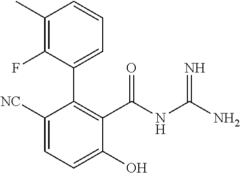 [SMILES: Cc1cccc(-c2c(C#N)ccc(O)c2C(=O)NC(=N)N)c1F] | N-carbamimidoyl-6-cyano-2′-fluoro-3-hydroxy- 3′-methyl-[1,1′-biphenyl]-2-carboxamide LC-MS: m/z 313 (M + H)⁺. 1H NMR (400 MHz, DMSO) δ 7.82 (br s, 2H), 7.63 (d, J = 8.8 Hz, 1H), 7.23 (t, J = 7.2 Hz, 1H), 7.07 (t, J = 7.6 Hz, 1H), 6.99 (t, J = 7.2 Hz, 1H), 6.87 (d, J = 8.8 Hz, 1H), 2.25 (s, 3H). |
| 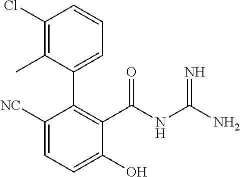 [SMILES: Cc1c(Cl)cccc1-c1c(C#N)ccc(O)c1C(=O)NC(=N)N] | N-carbamimidoyl-3′-chloro-6-cyano-3-hydroxy- 2′-methyl-[1,1′-biphenyl]-2-carboxamide LC-MS: m/z 329 (M + H)⁺. 1H NMR (400 MHz, DMSO) δ 7.59 (d, J = 8.7 Hz, 1H), 7.35 (d, J = 7.9 Hz, 1H), 7.18 (t, J = 7.7 Hz, 1H), 6.93 (d, J = 7.5 Hz, 1H), 6.82 (d, J = 8.7 Hz, 1H), 2.00 (s, 3H). |
| 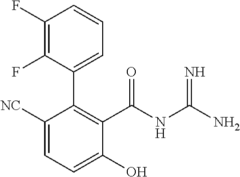 [SMILES: N#Cc1ccc(O)c(C(=O)NC(=N)N)c1-c1cccc(F)c1F] | N-carbamimidoyl-6-cyano-2′,3′-difluoro-3- hydroxy-[1,1′-biphenyl]-2-carboxamide LC-MS: m/z 317 (M + H)⁺. 1H NMR (400 MHz, DMSO) δ: 8.26 (br s, 1H), 7.67 (d, J = 8.8 Hz, 1H), 7.45-7.34 (m, 1H), 7.20 (dd, J = 12.6, 7.6 Hz, 1H), 7.03 (dd, J = 7.6, 6.3 Hz, 1H), 6.91 (d, J = 8.8 Hz, 1H). |
| 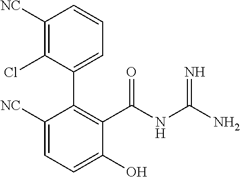 [SMILES: N#Cc1cccc(-c2c(C#N)ccc(O)c2C(=O)NC(=N)N)c1Cl] | N-carbamimidoyl-2′-chloro-3′,6-dicyano-3- hydroxy-[1,1′-biphenyl]-2-carboxamide LC-MS: m/z 340 (M + H)⁺. 1H NMR (400 MHz, DMSO) δ: 8.27 (br s, 1H), 7.96 (dd, J = 7.1, 2.2 Hz, 1H), 7.70 (d, J = 8.8 Hz, 1H), 7.63-7.54 (m, 2H), 7.26 (br s, 1H), 6.93 (d, J = 8.8 Hz, 1H). |
| 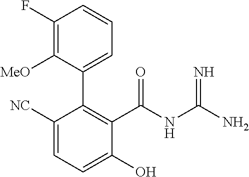 [SMILES: COc1c(F)cccc1-c1c(C#N)ccc(O)c1C(=O)NC(=N)N] | N-carbamimidoyl-6-cyano-3′-fluoro-3-hydroxy- 2′-methoxy-[1,1′-biphenyl]-2-carboxamide LC-MS: m/z 329 (M + H)⁺. 1H NMR (400 MHz, DMSO) δ 7.62 (d, J = 8.7 Hz, 1H), 7.20 (ddd, J = 11.9, 8.3, 1.5 Hz, 1H), 7.06 (td, J = 8.0, 5.1 Hz, 1H), 6.86 (d, J = 8.7 Hz, 2H), 3.63 (d, J = 1.4 Hz, 3H). |
| 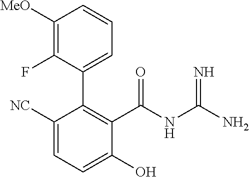 [SMILES: COc1cccc(-c2c(C#N)ccc(O)c2C(=O)NC(=N)N)c1F] | N-carbamimidoyl-6-cyano-2′-fluoro-3-hydroxy- 3′-methoxy-[1,1′-biphenyl]-2-carboxamide LC-MS: m/z 329 (M + H)⁺. 1H NMR (400 MHz, DMSO) δ 8.29 (br s, 2H), 7.63 (d, J = 8.7 Hz, 1H), 7.22 (br s, 1H), 7.12- 7.07 (m, 2H), 6.88 (d, J = 8.7 Hz, 1H), 6.74- 6.68 (m, 1H), 3.85 (s, 3H). |
| 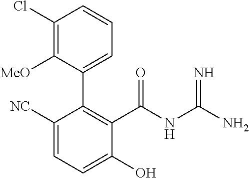 [SMILES: COc1c(Cl)cccc1-c1c(C#N)ccc(O)c1C(=O)NC(=N)N] | N-carbamimidoyl-3′-chloro-6-cyano-3-hydroxy- 2′-methoxy-[1,1′-biphenyl]-2-carboxamide LC-MS: m/z 345 (M + H)⁺. 1H NMR (400 MHz, DMSO) δ 8.21 (br s, 1H), 7.64 (d, J = 8.8 Hz, 1H), 7.42 (dd, J = 8.0, 1.6 Hz, 1H), 7.12 (t, J = 8.0 Hz, 1H), 7.03 (dd, J = 7.6, 1.6 Hz, 1H), 6.88 (d, J = 8.8 Hz, 1H), 3.49 (s, 3H). |
| 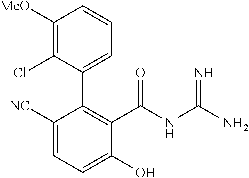 [SMILES: COc1cccc(-c2c(C#N)ccc(O)c2C(=O)NC(=N)N)c1Cl] | N-carbamimidoyl-2′-chloro-6-cyano-3-hydroxy- 3′-methoxy-[1,1′-biphenyl]-2-carboxamide LC-MS: m/z 345 (M + H)⁺. 1H NMR (400 MHz, DMSO) δ: 8.29 (br s, 2H), 7.62 (d, J = 8.7 Hz, 1H), 7.27 (br s, 1H), 7.07 (dd, J = 8.4, 1.3 Hz, 1H), 6.86 (d, J = 8.7 Hz, 1H), 6.75 (dd, J = 7.6, 1.3 Hz, 1H), 3.88 (s, 3H). |
| 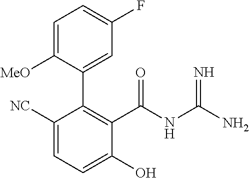 [SMILES: COc1ccc(F)cc1-c1c(C#N)ccc(O)c1C(=O)NC(=N)N] | N-carbamimidoyl-6-cyano-5′-fluoro-3-hydroxy- 2′-methoxy-[1,1′-biphenyl]-2-carboxamide LC-MS: m/z 329 (M + H)⁺. 1H NMR (400 MHz, DMSO) δ 8.23 (br s, 2H), 7.59 (d, J = 8.7 Hz, 1H), 7.09 (t, J = 8.5 Hz, 1H), 6.99 (dd, J = 8.8, 4.5 Hz, 1H), 6.87 (d, J = 8.7 Hz, 1H), 6.83 (d, J = 8.8 Hz, 1H), 3.64 (s, 3H). |
| 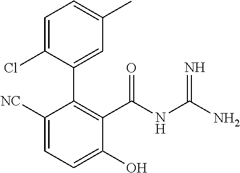 [SMILES: Cc1ccc(Cl)c(-c2c(C#N)ccc(O)c2C(=O)NC(=N)N)c1] | N-carbamimidoyl-2′-chloro-6-cyano-3-hydroxy- 5′-methyl-[1,1′-biphenyl]-2-carboxamide LC-MS: m/z 329 (M + H)⁺. 1H NMR (400 MHz, DMSO) δ 7.56 (d, J = 8.8 Hz, 1H), 7.30 (d, J = 8.0 Hz, 1H), 7.12 (d, J = 8.0 Hz, 1H), 6.98 (s, 1H), 6.79 (d, J = 8.0 Hz, 1H), 2.29 (s, 3H). |
| 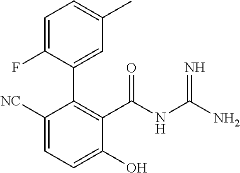 [SMILES: Cc1ccc(F)c(-c2c(C#N)ccc(O)c2C(=O)NC(=N)N)c1] | N-carbamimidoyl-6-cyano-2′-fluoro-3-hydroxy- 5′-methyl-[1,1′-biphenyl]-2-carboxamide LC-MS: m/z 313 (M + H)⁺. 1H NMR (400 MHz, DMSO) δ 7.80 (br s, 2H), 7.63 (d, J = 8.8 Hz, 1H), 7.19-7.12 (m, 1H), 7.05 (t, J = 9.2 Hz, 1H), 6.98 (d, J = 7.2 Hz, 1H), 6.87 (d, J = 8.4 Hz, 1H), 2.29 (s, 3H). |
| 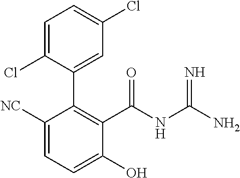 [SMILES: N#Cc1ccc(O)c(C(=O)NC(=N)N)c1-c1cc(Cl)ccc1Cl] | N-carbamimidoyl-2′,5′-dichloro-6-cyano-3- hydroxy-[1,1′-biphenyl]-2-carboxamide LC-MS: m/z 349 (M + H)⁺. 1H NMR (400 MHz, DMSO) δ 8.31 (br s, 2H), 7.66 (d, J = 8.8 Hz, 1H), 7.50 (d, J = 8.4 Hz, 1H), 7.41 (d, J = 8.8 Hz, 1H), 7.33 (s, 1H), 7.24 (br s 1H), 6.89 (d, J = 8.8 Hz, 1H). |
| 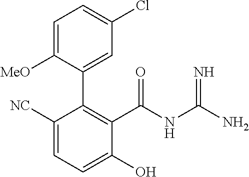 [SMILES: COc1ccc(Cl)cc1-c1c(C#N)ccc(O)c1C(=O)NC(=N)N] | N-carbamimidoyl-5′-chloro-6-cyano-3-hydroxy- 2′-methoxy-[1,1′-biphenyl]-2-carboxamide LC-MS: m/z 345 (M + H)⁺. 1H NMR (400 MHz, DMSO) δ 7.56 (d, J = 8.7 Hz, 1H), 7.31 (d, J = 8.9 Hz, 1H), 7.02 (d, J = 8.3 Hz, 2H), 6.79 (d, J = 8.7 Hz, 1H), 3.65 (s, 3H). |
| 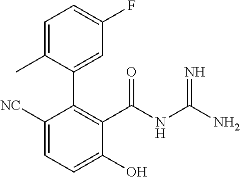 [SMILES: Cc1ccc(F)cc1-c1c(C#N)ccc(O)c1C(=O)NC(=N)N] | N-carbamimidoyl-6-cyano-5′-fluoro-3-hydroxy- 2′-methyl-[1,1′-biphenyl]-2-carboxamide LC-MS: m/z 313 (M + H)⁺. 1H NMR (400 MHz, DMSO) δ 7.79 (br s, 2H), 7.60 (d, J = 8.8 Hz, 1H), 7.21 (t, J = 7.6 Hz, 1H), 7.00 (t, J = 8.4 Hz, 1H), 6.82 (d, J = 9.2 Hz, 2H), 1.92 (s, 3H). |
| 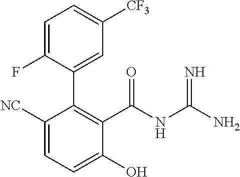 [SMILES: N#Cc1ccc(O)c(C(=O)NC(=N)N)c1-c1cc(C(F)(F)F)ccc1F] | N-carbamimidoyl-6-cyano-2′-fluoro-3-hydroxy- 5′-(trifluoromethyl)-[1,1′-biphenyl]-2- carboxamide LC-MS: m/z 367 (M + H)⁺. 1H NMR (400 MHz, DMSO) δ 7.81-7.77 (m, 1H), 7.69 (d, J = 8.8 Hz, 1H), 7.65 (dd, J = 6.4, 2.0 Hz, 1H), 7.46 (t, J = 8.8 Hz, 1H), 6.93 (d, J = 8.8 Hz, 1H). |
| 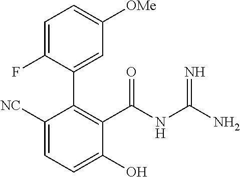 [SMILES: COc1ccc(F)c(-c2c(C#N)ccc(O)c2C(=O)NC(=N)N)c1] | N-carbamimidoyl-6-cyano-2′-fluoro-3-hydroxy- 5′-methoxy-[1,1′-biphenyl]-2-carboxamide LC-MS: m/z 329 (M + H)⁺. 1H NMR (400 MHz, DMSO) δ 8.29 (br s, 2H), 7.64 (d, J = 8.8 Hz, 1H), 7.10 (t, J = 8.9 Hz, 1H), 6.88 (d, J = 8.7 Hz, 2H), 6.73 (s, 1H), 3.73 (s, 3H). |
| 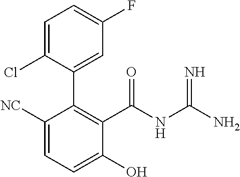 [SMILES: N#Cc1ccc(O)c(C(=O)NC(=N)N)c1-c1cc(F)ccc1Cl] | N-carbamimidoyl-2′-chloro-6-cyano-5′-fluoro-3- hydroxy-[1,1′-biphenyl]-2-carboxamide LC-MS: m/z 333 (M + H)⁺. 1H NMR (400 MHz, DMSO) δ 8.29 (br s, 2H), 7.64 (d, J = 8.0 Hz, 1H), 7.50 (dd, J = 8.8, 5.1 Hz, 1H), 7.22-7.14 (m, 2H), 6.89 (d, J = 8.8 Hz, 1H). |
| 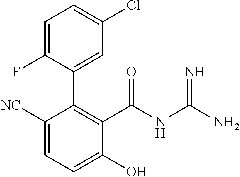 [SMILES: N#Cc1ccc(O)c(C(=O)NC(=N)N)c1-c1cc(Cl)ccc1F] | N-carbamimidoyl-5′-chloro-6-cyano-2′-fluoro-3- hydroxy-[1,1′-biphenyl]-2-carboxamide LC-MS: m/z 333 (M + H)⁺. 1H NMR (400 MHz, DMSO) δ: 7.66 (d, J = 8.8 Hz, 1H), 7.43 (ddd, J = 8.7, 4.4, 2.7 Hz, 1H), 7.32 (dd, J = 6.3, 2.7 Hz, 1H), 7.26 (t, J = 9.0 Hz, 1H), 6.90 (d, J = 8.8 Hz, 1H). |
| 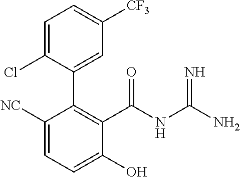 [SMILES: N#Cc1ccc(O)c(C(=O)NC(=N)N)c1-c1cc(C(F)(F)F)ccc1Cl] | N-carbamimidoyl-2′-chloro-6-cyano-3-hydroxy- 5′-(trifluoromethyl)-[1,1′-biphenyl]-2- carboxamide LC-MS: m/z 383 (M + H)⁺. 1H NMR (400 MHz, DMSO) δ 8.33 (br s, 2H), 7.73 (t, J = 4.3 Hz, 2H), 7.69 (d, J = 8.8 Hz, 1H), 7.61 (s, 1H), 7.32 (br s, 1H), 6.92 (d, J = 8.8 Hz, 1H). |
| 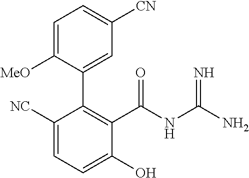 [SMILES: COc1ccc(C#N)cc1-c1c(C#N)ccc(O)c1C(=O)NC(=N)N] | N-carbamimidoyl-5′,6-dicyano-3-hydroxy-2′- methoxy-[1,1′-biphenyl]-2-carboxamide LC-MS: m/z 336 (M + H)⁺. 1H NMR (400 MHz, DMSO) δ 8.26 (br s, 2H), 7.80 (dd, J = 8.4, 2.0 Hz, 1H), 7.62 (d, J = 8.4 Hz, 1H), 7.49 (d, J = 2.4 Hz, 1H), 7.20 (d, J = 8.8 Hz, 1H), 6.86 (d, J = 8.4 Hz, 1H), 3.75 (s, 3H). |
| 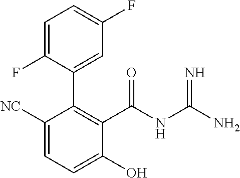 [SMILES: N#Cc1ccc(O)c(C(=O)NC(=N)N)c1-c1cc(F)ccc1F] | N-carbamimidoyl-6-cyano-2′,5′-difluoro-3- hydroxy-[1,1′-biphenyl]-2-carboxamide LC-MS: m/z 317 (M + H)⁺. 1H NMR (400 MHz, DMSO) δ: 8.32 (br s, 2H), 7.66 (d, J = 8.8 Hz, 1H), 7.32-7.10 (m, 3H), 6.90 (d, J = 8.8 Hz, 1H). |
| 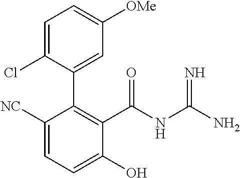 [SMILES: COc1ccc(Cl)c(-c2c(C#N)ccc(O)c2C(=O)NC(=N)N)c1] | N-carbamimidoyl-2′-chloro-6-cyano-3-hydroxy- 5′-methoxy-[1,1′-biphenyl]-2-carboxamide LC-MS: m/z 345 (M + H)⁺. 1H NMR (400 MHz, DMSO) δ 8.29 (br s, 2H), 7.64 (d, J = 8.7 Hz, 1H), 7.34 (d, J = 8.8 Hz, 1H), 6.94-6.84 (m, 2H), 6.76 (d, J = 3.0 Hz, 1H), 3.74 (s, 3H). |
| 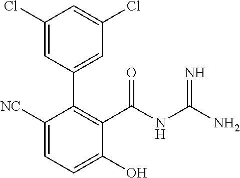 [SMILES: N#Cc1ccc(O)c(C(=O)NC(=N)N)c1-c1cc(Cl)cc(Cl)c1] | N-carbamimidoyl-3′,5′-dichloro-6-cyano-3- hydroxy-[1,1′-biphenyl]-2-carboxamide LC-MS: m/z 349 (M + H)⁺. 1H NMR (400 MHz, DMSO) δ 8.44 (br s, 2H), 7.56 (d, J = 8.8 Hz, 1H), 7.16 (t, J = 9.5 Hz, 1H), 6.93 (d, J = 7.4 Hz, 2H), 6.80 (d, J = 8.8 Hz, 1H). |
| 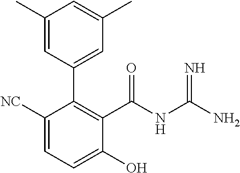 [SMILES: Cc1cc(C)cc(-c2c(C#N)ccc(O)c2C(=O)NC(=N)N)c1] | N-carbamimidoyl-6-cyano-3-hydroxy-3′,5′- dimethyl-[1,1′-biphenyl]-2-carboxamide LC-MS: m/z 309 (M + H)⁺. 1H NMR (400 MHz, DMSO) δ 8.17 (br s, 2H), 7.56 (d, J = 8.8 Hz, 1H), 7.20 (br s, 1H), 6.92 (s, 1H), 6.82 (d, J = 8.8 Hz, 1H), 6.74 (s, 2H), 2.27 (s, 6H). |
| 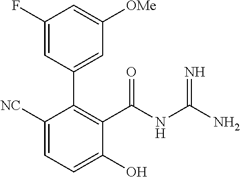 [SMILES: COc1cc(F)cc(-c2c(C#N)ccc(O)c2C(=O)NC(=N)N)c1] | N-carbamimidoyl-6-cyano-3′-fluoro-3-hydroxy- 5′-methoxy-[1,1′-biphenyl]-2-carboxamide LC-MS: m/z 329 (M + H)⁺. 1H NMR (400 MHz, DMSO) δ 8.08 (br s, 2H), 7.52 (d, J = 8.4 Hz, 1H), 6.85-6.68 (m, 2H), 6.57 (d, J = 10.4 Hz, 2H), 3.76 (s, 3H). |
| 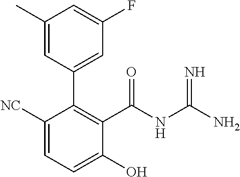 [SMILES: Cc1cc(F)cc(-c2c(C#N)ccc(O)c2C(=O)NC(=N)N)c1] | N-carbamimidoyl-6-cyano-3′-fluoro-3-hydroxy- 5′-methyl-[1,1′-biphenyl]-2-carboxamide LC-MS: m/z 313 (M + H)⁺. 1H NMR (400 MHz, DMSO) δ 7.56 (d, J = 8.7 Hz, 1H), 6.95 (d, J = 9.9 Hz, 1H), 6.84-6.75 (m, 3H), 2.32 (s, 3H). |
| 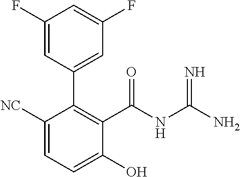 [SMILES: N#Cc1ccc(O)c(C(=O)NC(=N)N)c1-c1cc(F)cc(F)c1] | N-carbamimidoyl-6-cyano-3′,5′-difluoro-3- hydroxy-[1,1′-biphenyl]-2-carboxamide LC-MS: m/z 317 (M + H)⁺. 1H NMR (400 MHz, DMSO) δ 8.28 (s, 1H), 7.62 (d, J = 8.7 Hz, 1H), 7.56-7.52 (m, 1H), 7.28-7.21 (m, 2H), 6.87 (d, J = 8.7 Hz, 1H). |
| 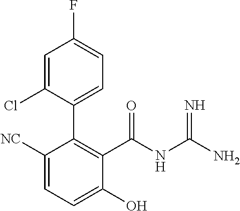 [SMILES: N#Cc1ccc(O)c(C(=O)NC(=N)N)c1-c1ccc(F)cc1Cl] | N-carbamimidoyl-2′-chloro-6-cyano-4′-fluoro-3- hydroxy-1,1′-biphenyl-2-carboxamide LC-MS: m/z 333 (M + H)⁺. 1H NMR (400 MHz, DMSO) δ 7.81 (br s, 2H), 7.64 (d, J = 8.8 Hz, 1H), 7.45 (dd, J = 9.0, 2.3 Hz, 1H), 7.28-7.18 (m, 2H), 6.87 (d, J = 8.7 Hz, 1H). |
| 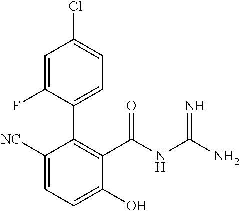 [SMILES: N#Cc1ccc(O)c(C(=O)NC(=N)N)c1-c1ccc(Cl)cc1F] | N-carbamimidoyl-4′-chloro-6-cyano-2′-fluoro-3- hydroxy-1,1′-biphenyl-2-carboxamide LC-MS: m/z 301 (M + H)⁺. 1H NMR (400 MHz, DMSO) δ 7.67 (d, J = 8.8 Hz, 1H), 7.44 (dd, J = 9.6, 1.8 Hz, 1H), 7.27 (br s, 2H), 6.90 (d, J = 8.8 Hz, 1H). |
| 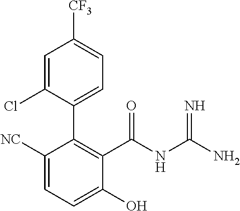 [SMILES: N#Cc1ccc(O)c(C(=O)NC(=N)N)c1-c1ccc(C(F)(F)F)cc1Cl] | N-carbamimidoyl-2′-chloro-6-cyano-3-hydroxy- 4′-(trifluoromethyl)-[1,1′-biphenyl]-2- carboxamide LC-MS: m/z 383 (M + H)⁺. NMR (400 MHz, DMSO) δ 8.30 (br s, 2H), 7.91 (s, 1H), 7.73 (d, J = 7.6 Hz, 1H), 7.70 (d, J = 8.8 Hz, 1H), 7.47 (d, J = 8.0 Hz, 1H), 6.92 (d, J = 8.8 Hz, 1H). |
| 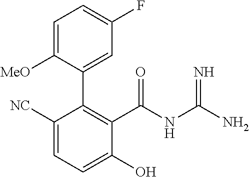 [SMILES: COc1ccc(F)cc1-c1c(C#N)ccc(O)c1C(=O)NC(=N)N] | N-carbamimidoyl-6-cyano-5′-fluoro-3-hydroxy- 2′-methoxy-[1,1′-biphenyl]-2-carboxamide LC-MS: m/z 329 (M + H)⁺. 1H NMR (400 MHz, DMSO) δ 8.23 (br s, 2H), 7.59 (d, J = 8.7 Hz, 1H), 7.09 (t, J = 8.5 Hz, 1H), 6.99 (dd, J = 8.8, 4.5 Hz, 1H), 6.87 (d, J = 8.7 Hz, 1H), 6.83 (d, J = 8.8 Hz, 1H), 3.64 (s, 3H). |
| 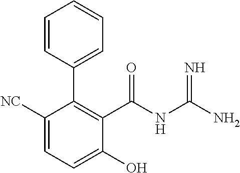 [SMILES: N#Cc1ccc(O)c(C(=O)NC(=N)N)c1-c1ccccc1] | N-carbamimidoyl-3-hydroxy-6- (trifluoromethyl)-[1,1′-biphenyl]-2-carboxamide LC-MS: m/z 324 (M + H)⁺. 1H NMR (400 MHz, DMSO) δ 8.18 (s, 1H), 7.99 (br s, 1H), 7.59 (d, J = 8.9 Hz, 1H), 7.32- 7.19 (m, 3H), 7.08 (d, J = 7.4 Hz, 2H), 6.92 (d, J = 8.8 Hz, 1H). |
| 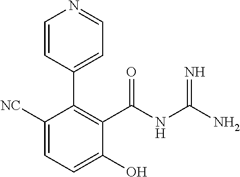 [SMILES: N#Cc1ccc(O)c(C(=O)NC(=N)N)c1-c1ccncc1] | N-carbamimidoyl-3-cyano-6-hydroxy-2- (pyridin-4-yl)benzamide LC-MS: m/z 282 (M + H)⁺. 1H NMR (400 MHz, DMSO-d6) δ 8.54 (dd, J = 4.5, 1.4 Hz, 2H), 7.61 (d, J = 8.8 Hz, 1H), 7.20 (dd, J = 4.5, 1.5 Hz, 2H), 6.85 (d, J = 8.8 Hz, 1H). |
| 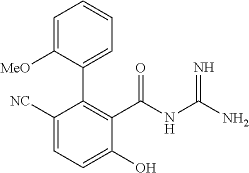 [SMILES: COc1ccccc1-c1c(C#N)ccc(O)c1C(=O)NC(=N)N] | N-carbamimidoyl-6-cyano-3-hydroxy-2′- methoxy-[1,1′-biphenyl]-2-carboxamide LC-MS: m/z 311 (M + H)⁺. 1H NMR (400 MHz, DMSO-d6) δ 8.20 (br s, 1H), 7.60 (d, J = 8.7 Hz, 1H), 7.29 (t, J = 7.0 Hz, 1H), 7.03-6.96 (m, 2H), 6.93 (t, J = 7.3 Hz, 1H), 6.84 (d, J = 8.6 Hz, 1H), 3.66 (s, 3H). |
| 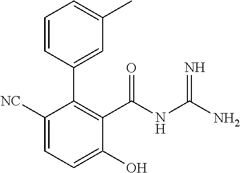 [SMILES: Cc1cccc(-c2c(C#N)ccc(O)c2C(=O)NC(=N)N)c1] | N-carbamimidoyl-6-cyano-3-hydroxy-3′-methyl- [1,1′-biphenyl]-2-carboxamide LC-MS: m/z 295 (M + H)⁺. 1H NMR (400 MHz, DMSO-d6) δ 7.56 (d, J = 8.7 Hz, 1H), 7.22 (t, J = 7.5 Hz, 1H), 7.11 (d, J = 7.6 Hz, 1H), 6.98-6.91 (m, 2H), 6.81 (d, J = 8.7 Hz, 1H), 2.31 (s, 3H). |
| 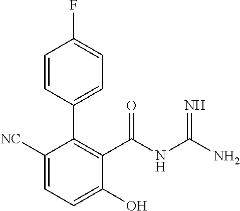 [SMILES: N#Cc1ccc(O)c(C(=O)NC(=N)N)c1-c1ccc(F)cc1] | N-carbamimidoyl-6-cyano-4′-fluoro-3-hydroxy- [1,1′-biphenyl]-2-carboxamide LC-MS: m/z 299 (M + H)⁺. 1H NMR (400 MHz, DMSO-d6) δ 7.56 (d, J = 8.7 Hz, 1H), 7.22-7.14 (m, 4H), 6.81 (d, J = 8.6 Hz, 1H). |
| 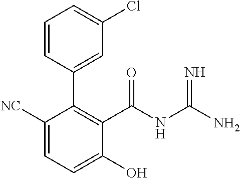 [SMILES: N#Cc1ccc(O)c(C(=O)NC(=N)N)c1-c1cccc(Cl)c1] | N-carbamimidoyl-3′-chloro-6-cyano-3-hydroxy- [1,1′-biphenyl]-2-carboxamide LC-MS: m/z 315 (M + H)⁺. 1H NMR (400 MHz DMSO-d6) δ 7.58 (d J = 8.8 Hz, 1H), 7.39-7.32 (m, 2H), 7.22 (s, 1H), 7.14-7.09 (m, 1H), 6.83 (d, J = 8.7 Hz, 1H). |
| 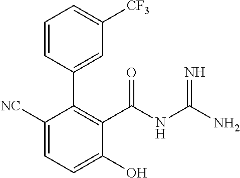 [SMILES: N#Cc1ccc(O)c(C(=O)NC(=N)N)c1-c1cccc(C(F)(F)F)c1] | N-carbamimidoyl-6-cyano-3-hydroxy-3′- (trifluoromethyl)-[1,1′-biphenyl]-2-carboxamide LC-MS: m/z 349 (M + H)⁺. 1H NMR (400 MHz, DMSO-d6) δ 7.68 (d, J = 7.7 Hz, 1H), 7.63-7.56 (m, 2H), 7.53-7.45 (m, 2H), 6.87 (d, J = 8.8 Hz, 1H). |
| 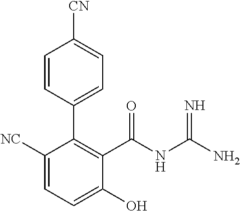 [SMILES: N#Cc1ccc(-c2c(C#N)ccc(O)c2C(=O)NC(=N)N)cc1] | N-carbamimidoyl-4′,6-dicyano-3-hydroxy-[1,1′- biphenyl]-2-carboxamide LC-MS: m/z 306 (M + H)⁺. 1H NMR (400 MHz, DMSO-d6) δ 7.81 (d, J = 8.3 Hz, 2H), 7.58 (d, J = 8.7 Hz, 1H), 7.42-7.33 (m, 2H), 6.82 (d, J = 8.7 Hz, 1H). |
| 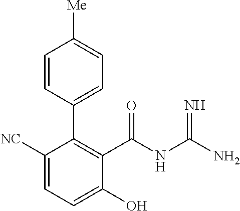 [SMILES: Cc1ccc(-c2c(C#N)ccc(O)c2C(=O)NC(=N)N)cc1] | N-carbamimidoyl-6-cyano-3-hydroxy-4′-methyl- [1,1′-biphenyl]-2-carboxamide LC-MS: m/z 295 (M + H)⁺. 1H NMR (400 MHz, DMSO-d6) δ 7.54 (d, J = 8.6 Hz, 1H), 7.13 (d, J = 7.9 Hz, 2H), 7.03 (d, J = 7.9 Hz, 2H), 6.78 (d, J = 8.5 Hz, 1H), 2.33 (s, 3H). |
| 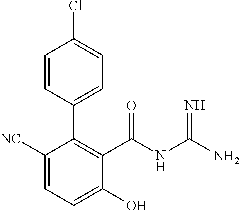 [SMILES: N#Cc1ccc(O)c(C(=O)NC(=N)N)c1-c1ccc(Cl)cc1] | N-carbamimidoyl-4′-chloro-6-cyano-3-hydroxy- [1,1′-biphenyl]-2-carboxamide LC-MS: m/z 315 (M + H)⁺. 1H NMR (400 MHz, DMSO-d6) δ 7.61 (d, J = 8.7 Hz, 1H), 7.40 (d, J = 8.3 Hz, 2H), 7.19 (d, J = 8.3 Hz, 2H), 6.86 (d, J = 8.7 Hz, 1H). |
| 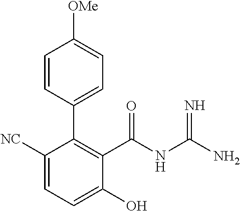 [SMILES: COc1ccc(-c2c(C#N)ccc(O)c2C(=O)NC(=N)N)cc1] | N-carbamimidoyl-6-cyano-3-hydroxy-4′- methoxy-[1,1′-biphenyl]-2-carboxamide LC-MS: m/z 311 (M + H)⁺. 1H NMR (400 MHz, DMSO-d6) δ 7.57 (d, J = 8.7 Hz, 1H), 7.08 (d, J = 8.5 Hz, 2H), 6.90 (d, J = 8.6 Hz, 2H), 6.82 (d, J = 8.7 Hz, 1H), 3.79 (s, 3H). |
| 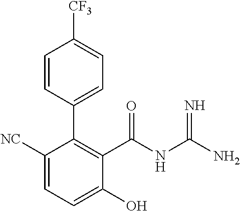 [SMILES: N#Cc1ccc(O)c(C(=O)NC(=N)N)c1-c1ccc(C(F)(F)F)cc1] | N-carbamimidoyl-6-cyano-3-hydroxy-4′- (trifluoromethyl)-[1,1′-biphenyl]-2-carboxamide LC-MS: m/z 349 (M + H)⁺. 1H NMR (400 MHz, DMSO-d6) δ 7.71 (d, J = 8.2 Hz, 2H), 7.63 (d, J = 8.8 Hz, 1H), 7.39 (d, J = 8.0 Hz, 2H), 6.88 (d, J = 8.8 Hz, 1H). |
| 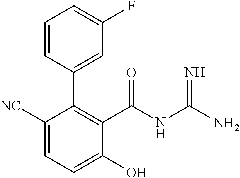 [SMILES: N#Cc1ccc(O)c(C(=O)NC(=N)N)c1-c1cccc(F)c1] | N-carbamimidoyl-6-cyano-3′-fluoro-3-hydroxy- [1,1′-biphenyl]-2-carboxamide LC-MS: m/z 299 (M + H)⁺. 1H NMR (400 MHz, DMSO-d6) δ 7.58 (d, J = 8.7 Hz, 1H), 7.38 (dd, J = 14.1, 7.9 Hz, 1H), 7.13 (td, J = 8.9, 2.5 Hz, 1H), 7.05-6.96 (m, 2H), 6.83 (d, J = 8.7 Hz, 1H). |
| 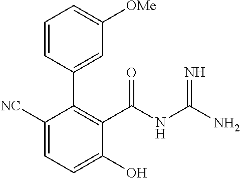 [SMILES: COc1cccc(-c2c(C#N)ccc(O)c2C(=O)NC(=N)N)c1] | N-carbamimidoyl-6-cyano-3-hydroxy-3′- methoxy-[1,1′-biphenyl]-2-carboxamide LC-MS: m/z 311 (M + H)⁺. 1H NMR (400 MHz, DMSO-d6) δ 7.55 (d, J = 8.7 Hz, 1H), 7.25 (t, J = 8.1 Hz, 1H), 6.89-6.83 (m, 1H), 6.80 (d, J = 8.7 Hz, 1H), 6.75-6.68 (m, 2H), 3.74 (s, 3H). |
| 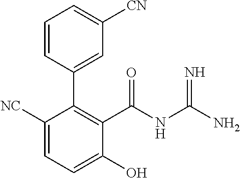 [SMILES: N#Cc1cccc(-c2c(C#N)ccc(O)c2C(=O)NC(=N)N)c1] | N-carbamimidoyl-3′,6-dicyano-3-hydroxy-[1,1′- biphenyl]-2-carboxamide LC-MS: m/z 306 (M + H)⁺. 1H NMR (400 MHz, DMSO-d6) δ 7.79 (d, J = 7.5 Hz, 1H), 7.69 (s, 1H), 7.63 (d, J = 8.7 Hz, 1H), 7.58-7.50 (m, 2H), 6.88 (d, J = 8.7 Hz, 1H). |
| 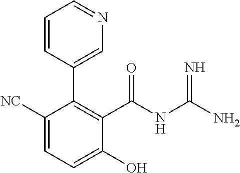 [SMILES: N#Cc1ccc(O)c(C(=O)NC(=N)N)c1-c1cccnc1] | N-carbamimidoyl-3-cyano-6-hydroxy-2- (pyridin-3-yl)benzamide LC-MS: m/z 282 (M + H)⁺. 1H NMR (400 MHz, DMSO-d6) δ 8.50 (d, J = 4.8 Hz, 1H), 8.35 (d, J = 2.1 Hz, 1H), 7.61 (d, J = 8.0 Hz, 2H), 7.42-7.34 (m, 1H), 6.85 (d, J = 8.8 Hz, 1H). |
| 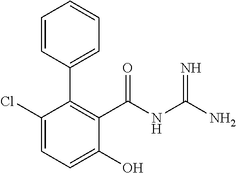 [SMILES: N=C(N)NC(=O)c1c(O)ccc(Cl)c1-c1ccccc1] | N-carbamimidoyl-6-chloro-3-hydroxy-[1,1′- biphenyl]-2-carboxamide LC-MS: m/z 290 (M + H)⁺. 1H NMR (400 MHz, DMSO) δ 15.36 (s, 1H), 8.02 (br s, 2H), 7.36 (d, J = 8.8 Hz, 1H), 7.33- 7.27 (m, 2H), 7.26-7.20 (m, 1H), 7.05-7.01 (m, 2H), 6.84 (d, J = 8.8 Hz, 1H). |
| 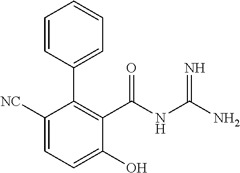 [SMILES: N#Cc1ccc(O)c(C(=O)NC(=N)N)c1-c1ccccc1] | N-carbamimidoyl-6-cyano-3-hydroxy-[1,1′- biphenyl]-2-carboxamide LC-MS: m/z 281 (M + H)⁺. 1H NMR (400 MHz, DMSO) δ 12.44 (br s, 1H), 8.27-8.12 (m, 4H), 7.88 (d, J = 8.8 Hz, 1H), 7.48-7.44 (m, 3H), 7.35-7.31 (m, 2H), 7.12 (d, J = 8.8 Hz, 1H). |

### General Procedure 8

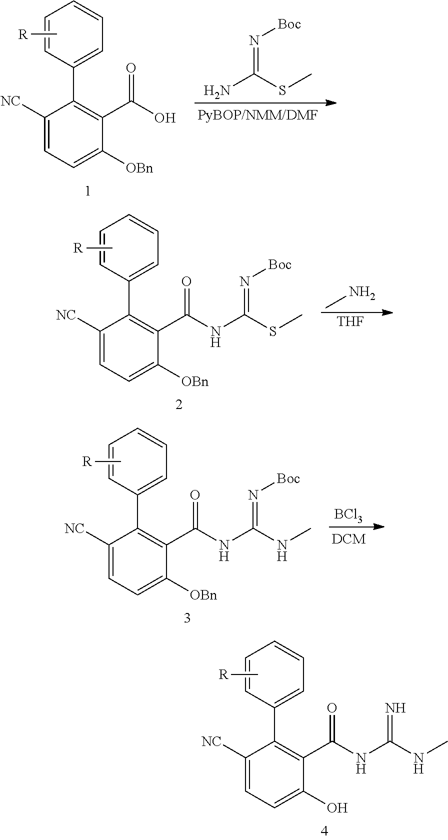

### Step 1: Synthesis of Compound 2

The synthesis of compound 2 was described in the general procedure 5.

### Step 2: Synthesis of Compound 3

Et₃N (5 eq.) was added to a solution of 2 (1 eq.) in THF followed by methylamine (3 eq.) and the resulting mixture was stirred at room temperature for 1 hour. The mixture was then poured into water and extracted with EtOAc. The organic layer was dried over anhydrous Na₂SO₄, filtered and concentrated under vacuum. The residue was purified by column chromatography on silica gel (eluted with Petroleum Ether:EA=3:1 to 1:1) to give compound 3.

### Step 3: Synthesis of Compound 4

BCl₃ (10 eq.) was added at −78° C. under N₂ atmosphere to a solution of 3 (1 eq.) in DCM and the resulting mixture was slowly warmed to 0° C. and stirred for 1 hour. The mixture was then quenched with MeOH (0.5 mL) and the solvent was removed under vacuum. The residue was purified via prep-HPLC (C18, 0% to 50% acetonitrile in H₂O with 0.1% NH₃.H₂O) to give compound 4.

### Synthesis of A365

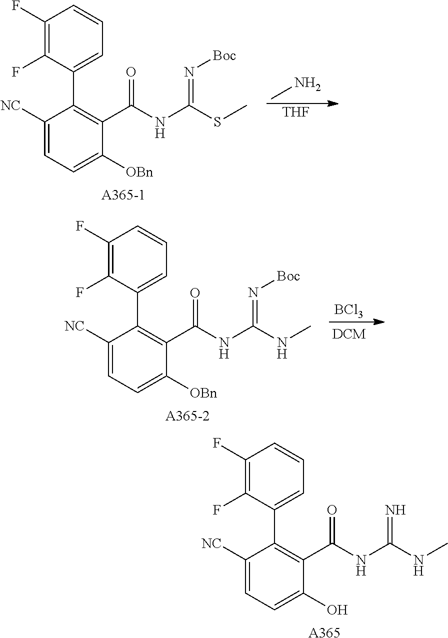

### Step 1: Synthesis of Compound A365-2

Et₃N (0.065 mL, 0.465 mmol) was added to a solution of A365-1 (50 mg, 0.0930 mmol) in THF (3 mL) followed by methylamine hydrochloride (19 mg, 0.279 mmol). The resulting mixture was stirred at room temperature for 1 hour. The mixture was then poured into water and extracted with EtOAc twice. The combined organic layers were dried over anhydrous Na₂SO₄, filtered and concentrated under vacuum. The residue was purified by column chromatography on silica gel (eluted with Petroleum Ether:EA=3:1 to 1:1) to give A365-2 (30 mg, 61.9719% yield) as a white solid. LC/MS (ESI) m/z: 521 (M+H)⁺.

### Step 3: Synthesis of Compound A365

BCl₃ (0.5760 mL, 1 M in DCM) was added at −78° C. under N₂ atmosphere to a solution of A365-2 (30 mg, 0.0576 mmol) in DCM (3 mL), and the resulting mixture was slowly warmed to 0° C. and stirred for 1 hour. The mixture was then quenched with MeOH (0.5 mL) and the solvent was removed under vacuum. The residue was purified via prep-HPLC (C18, 0% to 50% acetonitrile in H₂O with 0.1% NH₃.H₂O) to give A365 (3.5 mg, 18.4% yield) as a white solid. LC/MS (ESI) m/z: 331 (M+H)⁺. 1H NMR (400 MHz, DMSO) δ: 7.56 (d, J=8.8 Hz, 1H), 7.28-7.09 (m, 2H), 7.00-6.85 (m, 2H), 2.89 (d, J=37.6 Hz, 3H).

The compounds in the table below were prepared from the appropriate starting materials described previously or commercially available using the above general procedure 8.

| Structure | Name |
|---|---|
| 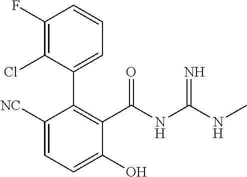 [SMILES: CNC(=N)NC(=O)c1c(O)ccc(C#N)c1-c1cccc(F)c1Cl] | 2′-chloro-6-cyano-3′-fluoro-3-hydroxy-N-(N- methylcarbamimidoyl)-[1,1-biphenyl]-2- carboxamide LC-MS: m/z 347 (M + H)⁺. 1H NMR (400 MHz, DMSO) δ 8.36 (br s, 2H), 7.67 (d, J = 8.8 Hz, 1H), 7.41-7.32 (m, 2H), 7.09- 6.99 (m, 1H), 6.91 (d, J = 8.7 Hz, 1H), 2.75 (s, 3H). |
| 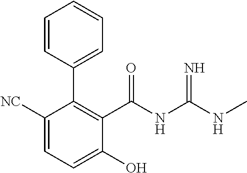 [SMILES: CNC(=N)NC(=O)c1c(O)ccc(C#N)c1-c1ccccc1] | 6-cyano-3-hydroxy-N-(N-methylcarbamimidoyl)- [1,1′-biphenyl]-2-carboxamide LC-MS: m/z 295 (M + H)⁺. 1H NMR (400 MHz, DMSO) δ 7.61 (d, J = 8.6 Hz, 1H), 7.39-7.29 (m, 3H), 7.16 (d, J = 6.7 Hz, 2H), 6.87 (d, J = 8.7 Hz, 1H), 2.73 (s, 3H). |
| 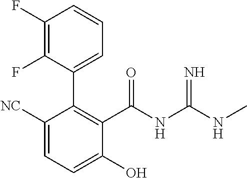 [SMILES: CNC(=N)NC(=O)c1c(O)ccc(C#N)c1-c1cccc(F)c1F] | 6-cyano-2′,3′-difluoro-3-hydroxy-N-(N- methylcarbamimidoyl)-[1,1′-biphenyl]-2- carboxamide LC-MS: m/z 331 (M + H)⁺. 1H NMR (400 MHz, DMSO) δ: 7.56 (d, J = 8.8 Hz, 1H), 7.28-7.09 (m, 2H), 7.00-6.85 (m, 2H), 2.89 (s, 3H). |
| 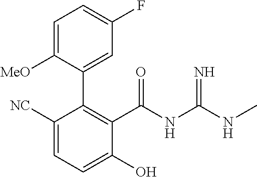 [SMILES: CNC(=N)NC(=O)c1c(O)ccc(C#N)c1-c1cc(F)ccc1OC] | 6-cyano-5′-fluoro-3-hydroxy-2′-methoxy-N-(N- methylcarbamimidoyl)-[1,1′-biphenyl]-2- carboxamide LC-MS: m/z 343 (M + H)⁺. 1H NMR (400 MHz, DMSO) δ 7.61 (d, J = 8.4 Hz, 1H), 7.10 (td, J = 8.7, 3.1 Hz, 1H), 7.00 (dd, J = 9.0, 4.6 Hz, 1H), 6.87 (dd, J = 12.5, 6.3 Hz, 2H), 3.64 (s, 3H), 2.72 (s, 3H). |
| 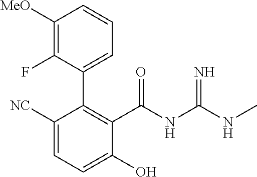 [SMILES: CNC(=N)NC(=O)c1c(O)ccc(C#N)c1-c1cccc(OC)c1F] | 6-cyano-2′-fluoro-3-hydroxy-3′-methoxy-N-(N- methylcarbamimidoyl)-[1,1′-biphenyl]-2- carboxamide LC-MS: m/z 343 (M + H)⁺. 1H NMR (400 MHz, DMSO) δ 8.96 (s, 1H), 8.31 (s, 1H), 7.64 (d, J = 8.6 Hz, 1H), 7.10 (d, J = 5.4 Hz, 2H), 6.89 (d, J = 8.9 Hz, 1H), 6.76-6.67 (m, 1H), 3.85 (s, 3H), 2.74 (s, 3H). |
| 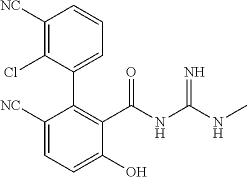 [SMILES: CNC(=N)NC(=O)c1c(O)ccc(C#N)c1-c1cccc(C#N)c1Cl] | 2′-chloro-3′,6-dicyano-3-hydroxy-N-(N- methylcarbamimidoyl)-[1,1′-biphenyl]-2- carboxamide LC-MS: m/z 354 (M + H)⁺. 1H NMR (400 MHz, DMSO) δ 7.96 (dd, J = 7.0, 2.3 Hz, 1H), 7.70 (d, J = 8.7 Hz, 1H), 7.62-7.54 (m, 2H), 6.93 (d, J = 8.8 Hz, 1H), 2.74 (s, 3H). |
| 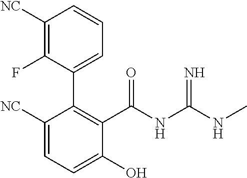 [SMILES: CNC(=N)NC(=O)c1c(O)ccc(C#N)c1-c1cccc(C#N)c1F] | 3′,6-dicyano-2′-fluoro-3-hydroxy-N-(N- methylcarbamimidoyl)-[1,1′-biphenyl]-2- carboxamide LC-MS: m/z 338 (M + H)⁺. 1H NMR (400 MHz, DMSO) δ 8.95 (s, 1H), 7.92 (t, J = 6.3 Hz, 1H), 7.71 (d, J = 8.8 Hz, 1H), 7.64 (t, J = 6.8 Hz, 1H), 7.44 (t, J = 7.7 Hz, 1H), 6.95 (d, J = 8.8 Hz, 1H), 2.75 (s, 3H). |
| 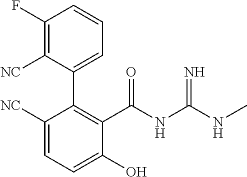 [SMILES: CNC(=N)NC(=O)c1c(O)ccc(C#N)c1-c1cccc(F)c1C#N] | 2′,6-dicyano-3′-fluoro-3-hydroxy-N-(N- methylcarbamimidoyl)-[1,1′-biphenyl]-2- carboxamide LC-MS: m/z 338 (M + H)⁺. 1H NMR (400 MHz, DMSO) δ: 8.96 (s, 1H), 8.48 (s, 1H), 7.85-7.70 (m, 2H), 7.51 (t, J = 8.9 Hz, 1H), 7.26 (d, J = 7.7 Hz, 1H), 6.97 (d, J = 8.8 Hz, 1H), 2.79 (s, 3H). |
| 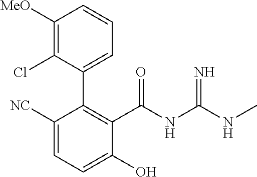 [SMILES: CNC(=N)NC(=O)c1c(O)ccc(C#N)c1-c1cccc(OC)c1Cl] | 2′-chloro-6-cyano-3-hydroxy-3′-methoxy-N-(N- methylcarbamimidoyl)-[1,1′-biphenyl]-2- carboxamide LC-MS: m/z 359 (M + H)⁺. 1H NMR (400 MHz, DMSO) δ 8.95 (s, 1H), 8.44 (br s, 1H), 7.63 (d, J = 8.4 Hz, 1H), 7.28 (t, J = 8.0 Hz, 1H), 7.08 (d, J = 8.0 Hz, 1H), 6.88 (d, J = 8.8 Hz, 1H), 6.75 (d, J = 6.8 Hz, 1H), 3.88 (s, 3H), 2.74 (s, 3H). |

### General Procedure 9

Combination of the general procedures 3 and 5 described above.

### Synthesis of A26

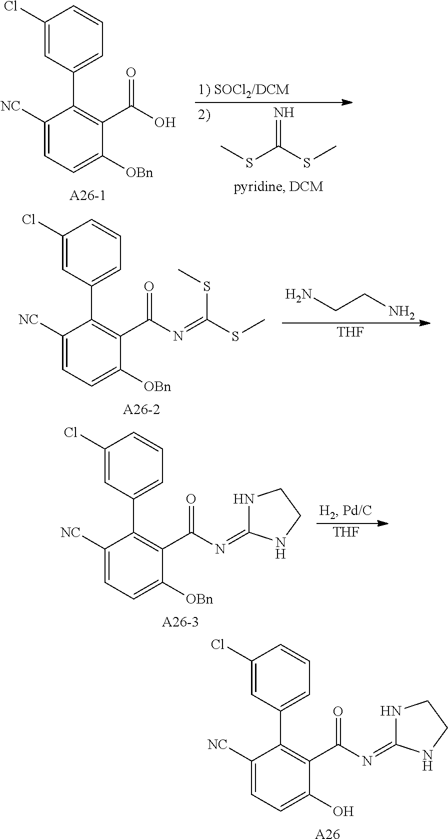

### Step 1: Synthesis of Compound A26-2

Thionyl chloride (2 mL, 27.57 mmol) was added to a solution of A26-1 (135 mg, 0.37 mmol) in DCM (10 mL) and the mixture was stirred 65° C. for 2 hours. The solvent was then removed under vacuum and the crude acyl chloride was dissolved in anhydrous DCM (2 mL) and added to a mixture of bis(methylsulfanyl)methanimine (67.4854 mg, 0.5567 mmol) and pyridine (494 mg, 1.86 mmol) in DCM (10 mL) at 0° C. The resulting mixture was stirred at room temperature for 30 minutes. The solvent was then removed under vacuum. The residue was purified by column chromatography on silica gel (eluted with Petroleum Ether:EA=10:1 to 1:1) to give A26-2 (83 mg, 47.9% yield) as a light yellow solid. LC/MS (ESI) m/z: 467 (M+H)⁺.

### Step 2: Synthesis of Compound A26-3

Ethane-1,2-diamine (0.048 mL, 0.71 mmol) was added to a solution of A26-2 (83 mg, 0.18 mmol) in THF (3 mL) and EtOH (3 mL), and the resulting mixture was heated to 80° C. for 1 hour. The solvents were then removed under vacuum and the residue was recrystallized from MeOH to give A26-3 (53 mg, 69.22% yield) as a white solid. LC/MS (ESI) m/z: 431 (M+H)⁺.

### Step 3: Synthesis of Compound A26-4

10% Pd/C (53 mg) was added under N₂ atmosphere to a solution of A26-3 (53 mg, 0.123 mmol) in THF (4 mL), and the resulting mixture was stirred at room temperature for 30 minutes under 15 psi H₂. The mixture was then filtered and the filtrate was concentrated under vacuum. The residue was purified via prep-HPLC (C18, 0% to 50% acetonitrile in H₂O with 0.1% NH₃.H₂O) to give A26 (14 mg, 33.4% yield) as a white solid. LC-MS: m/z 341 (M+H)⁺. 1H NMR (400 MHz, DMSO) δ: 8.55 (s, 2H), 7.68 (d, J=8.7 Hz, 1H), 7.43-7.34 (m, 2H), 7.25 (s, 1H), 7.16-7.11 (m, 1H), 6.95 (d, J=8.7 Hz, 1H), 3.53 (s, 4H).

The compounds in the table below were prepared from the appropriate starting materials described previously or commercially available using the above general procedure 9.

| Structure | Name |
|---|---|
| 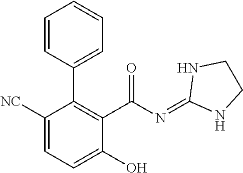 [SMILES: N#Cc1ccc(O)c(C(=O)N=C2NCCN2)c1-c1ccccc1] | 6-cyano-3-hydroxy-N-(imidazolidin-2-ylidene)- [1,1′-biphenyl]-2-carboxamide LC-MS: m/z 307 (M + H)⁺. 1H NMR (400 MHz, MeOD) δ 8.41 (br s, 2H), 7.58 (d, J = 8.4 Hz, 1H), 7.42-7.27 (m, 3H), 7.20 (d, J = 7.2 Hz, 2H), 6.93 (d, J = 8.8 Hz, 1H), 3.64 (s, 4H). |
| 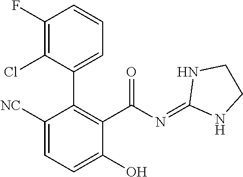 [SMILES: N#Cc1ccc(O)c(C(=O)N=C2NCCN2)c1-c1cccc(F)c1Cl] | 2′-chloro-6-cyano-3′-fluoro-3-hydroxy-N- (imidazolidin-2-ylidene)-[1,1′-biphenyl]-2- carboxamide LC-MS: m/z 359 (M + H)⁺. 1H NMR (400 MHz, DMSO) δ 8.57 (s, 2H), 7.73 (d, J = 8.7 Hz, 1H), 7.40-7.32 (m, 2H), 7.07 (d, J = 6.1 Hz, 1H), 6.98 (d, J = 8.7 Hz, 1H), 3.52 (s, 4H). |
| 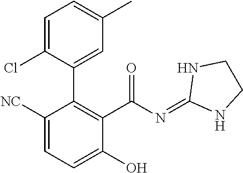 [SMILES: Cc1ccc(Cl)c(-c2c(C#N)ccc(O)c2C(=O)N=C2NCCN2)c1] | 2′-chloro-6-cyano-3-hydroxy-N-(imidazolidin-2- ylidene)-5′-methyl-[1,1′-biphenyl]-2- carboxamide LC-MS: m/z 355 (M + H)⁺. 1H NMR (400 MHz, DMSO) δ 8.52 (s, 2H), 7.69 (d, J = 8.4 Hz, 1H), 7.33 (d, J = 8.0 Hz, 1H), 7.14 (dd, J = 8.0, 1.6 Hz, 1H), 7.01 (d, J = 2.0 Hz, 1H), 6.95 (d, J = 8.8 Hz, 1H), 3.52 (s, 4H), 2.29 (s, 3H). |
| 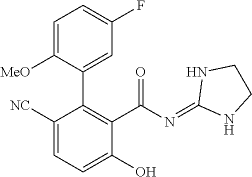 [SMILES: COc1ccc(F)cc1-c1c(C#N)ccc(O)c1C(=O)N=C1NCCN1] | 6-cyano-5′-fluoro-3-hydroxy-N-(imidazolidin-2- ylidene)-2′-methoxy-[1,1′-biphenyl]-2- carboxamide LC-MS: m/z 355 (M + H)⁺. 1H NMR (400 MHz, DMSO) δ 8.48 (s, 2H), 7.65 (d, J = 8.7 Hz, 1H), 7.09 (td, J = 8.7, 3.1 Hz, 1H), 7.00 (dd, J = 9.0, 4.6 Hz, 1H), 6.94-6.85 (m, 2H), 3.64 (s, 3H), 3.52 (s, 4H). |
| 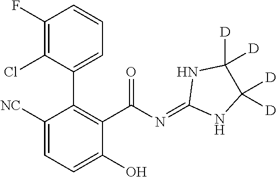 [SMILES: [2H]C1([2H])NC(=NC(=O)c2c(O)ccc(C#N)c2-c2cccc(F)c2Cl)NC1([2H])[2H]] | LC-MS: m/z 363 (M + H)⁺. 1H NMR (400 MHz, DMSO) δ 8.55 (s, 2H), 7.73 (d, J = 8.7 Hz, 1H), 7.42-7.32 (m, 2H), 7.09-7.03 (m, 1H), 6.98 (d, J = 8.7 Hz, 1H). |
| 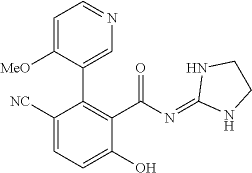 [SMILES: COc1ccncc1-c1c(C#N)ccc(O)c1C(=O)N=C1NCCN1] | 3-cyano-6-hydroxy-N-(imidazolidin-2-ylidene)- 2-(4-methoxypyridin-3-yl)benzamide LC-MS: m/z 338 (M + H)⁺. 1H NMR (400 MHz, DMSO) δ 8.52 (s, 2H), 8.42 (d, J = 6.0 Hz, 1H), 8.05 (s, 1H), 7.68 (d, J = 8.8 Hz, 1H), 7.09 (d, J = 5.6 Hz, 1H), 6.94 (d, J = 8.4 Hz, 1H), 3.75 (s, 3H), 3.53 (s, 4H). |
| 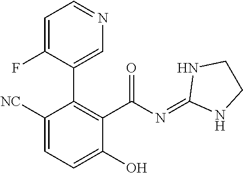 [SMILES: N#Cc1ccc(O)c(C(=O)N=C2NCCN2)c1-c1cnccc1F] | 3-cyano-2-(4-fluoropyridin-3-yl)-6-hydroxy-N- (imidazolidin-2-ylidene)benzamide LC-MS: m/z 326 (M + H)⁺. 1H NMR (400 MHz, DMSO) δ 8.52 (br s, 2H), 8.41 (d, J = 5.0 Hz, 1H), 7.78-7.68 (m, 2H), 7.50-7.43 (m, 1H), 7.03 (d, J = 8.7 Hz, 1H), 3.54 (s, 4H). |
| 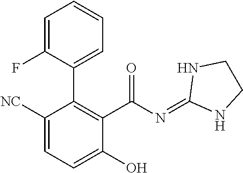 [SMILES: N#Cc1ccc(O)c(C(=O)N=C2NCCN2)c1-c1ccccc1F] | 6-cyano-2′-fluoro-3-hydroxy-N-(imidazolidin-2- ylidene)-[1,1'-biphenyl]-2-carboxamide LC-MS: m/z 325 (M + H)⁺. 1H NMR (400 MHz, DMSO) δ 8.54 (s, 2H), 7.71 (d, J = 8.7 Hz, 1H), 7.41-7.33 (m, 1H), 7.23-7.15 (m, 3H), 6.96 (d, J = 8.7 Hz, 1H), 3.52 (s, 4H). |
| 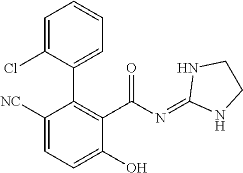 [SMILES: N#Cc1ccc(O)c(C(=O)N=C2NCCN2)c1-c1ccccc1Cl] | 2′-chloro-6-cyano-3-hydroxy-N-(imidazolidin-2- ylidene)-[1,1'-biphenyl]-2-carboxamide LC-MS: m/z 341 (M + H)⁺. 1H NMR (400 MHz, DMSO) δ 8.36 (br s, 2H), 7.65 (d, J = 8.7 Hz, 1H), 7.49-7.41 (m, 1H), 7.35-7.29 (m, 2H), 7.21-7.15 (m, 1H), 6.90 (d, J = 8.7 Hz, 1H), 3.49 (s, 4H). |
| 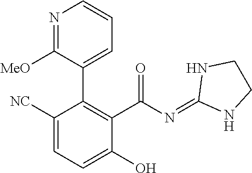 [SMILES: COc1ncccc1-c1c(C#N)ccc(O)c1C(=O)N=C1NCCN1] | 3-cyano-6-hydroxy-N-(imidazolidin-2-ylidene)- 2-(2-methoxypyridin-3-yl)benzamide LC-MS: m/z 338 (M + H)⁺. 1H NMR (400 MHz, DMSO) δ 8.50 (s, 2H), 8.11 (dd, J = 5.0, 1.9 Hz, 1H), 7.67 (d, J = 8.7 Hz, 1H), 7.41 (dd, J = 7.2, 1.9 Hz, 1H), 6.99 (dd, J = 7.2, 5.1 Hz, 1H), 6.91 (d, J = 8.7 Hz, 1H), 3.74 (s, 3H), 3.51 (s, 4H). |
| 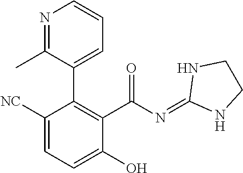 [SMILES: Cc1ncccc1-c1c(C#N)ccc(O)c1C(=O)N=C1NCCN1] | 3-cyano-6-hydroxy-N-(imidazolidin-2-ylidene)- 2-(2-methylpyridin-3-yl)benzamide LC-MS: m/z 322 (M + H)⁺. 1H NMR (400 MHz, DMSO) δ 8.54 (s, 2H), 8.38 (dd, J = 4.9, 1.7 Hz, 1H), 7.72 (d, J = 8.7 Hz, 1H), 7.37 (dd, J = 7.6, 1.7 Hz, 1H), 7.21 (dd, J = 7.6, 4.9 Hz, 1H), 6.96 (d, J = 8.7 Hz, 1H), 3.52 (s, 4H), 2.15 (s, 3H). |
| 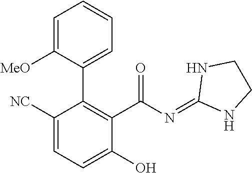 [SMILES: COc1ccccc1-c1c(C#N)ccc(O)c1C(=O)N=C1NCCN1] | 6-cyano-3-hydroxy-N-(imidazolidin-2-ylidene)- 2′-methoxy-[1,1′-biphenyl]-2-carboxamide LC-MS: m/z 427 (M + H)⁺. 1H NMR (400 MHz, DMSO) δ 8.48 (s, 2H), 7.63 (d, J = 8.6 Hz, 1H), 7.32-7.19 (m, 1H), 7.06-6.78 (m, 4H), 3.65 (s, 3H), 3.51 (s, 4H). |
| 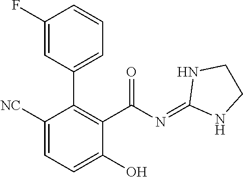 [SMILES: N#Cc1ccc(O)c(C(=O)N=C2NCCN2)c1-c1cccc(F)c1] | 6-cyano-3′-fluoro-3-hydroxy-N-(imidazolidin-2- ylidene)-[1,1'-biphenyl]-2-carboxamide LC-MS: m/z 325 (M + H)⁺. 1H NMR (400 MHz, DMSO) δ 8.54 (s, 2H), 7.67 (d, J = 8.7 Hz, 1H), 7.39 (td, J = 7.9, 6.2 Hz, 1H), 7.13 (td, J = 8.4, 2.2 Hz, 1H), 7.05-6.99 (m, 2H), 6.94 (d, J = 8.7 Hz, 1H), 3.52 (s, 4H). |
| 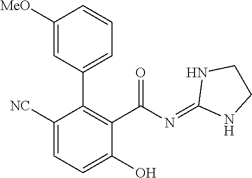 [SMILES: COc1cccc(-c2c(C#N)ccc(O)c2C(=O)N=C2NCCN2)c1] | 6-cyano-3-hydroxy-N-(imidazolidin-2-ylidene)- 3′-methoxy-[1,1′-biphenyl]-2-carboxamide LC-MS: m/z 337 (M + H)⁺. 1H NMR (400 MHz, DMSO) δ 8.52 (s, 2H), 7.64 (d, J = 8.7 Hz, 1H), 7.33-7.20 (m, 1H), 6.92 (d, J = 8.7 Hz, 2H), 6.87 (ddd, J = 8.3, 2.5, 0.9 Hz, 2H), 3.74 (s, 3H), 3.51 (s, 4H). |
| 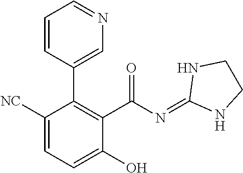 [SMILES: N#Cc1ccc(O)c(C(=O)N=C2NCCN2)c1-c1cccnc1] | 3-cyano-6-hydroxy-N-(imidazolidin-2-ylidene)- 2-(pyridin-3-yl)benzamide LC-MS: m/z 308 (M + H)⁺. 1H NMR (400 MHz, DMSO) δ 8.54 (br s, 2H), 8.51 (dd, J = 4.8, 1.6 Hz, 3H), 8.37 (d, J = 1.6 Hz, 1H), 7.71 (d, J = 8.7 Hz, 1H), 7.68-7.55 (m, 1H), 7.46-7.33 (m, 1H), 6.98 (d, J = 8.7 Hz, 1H), 3.52 (s, 4H). |
| 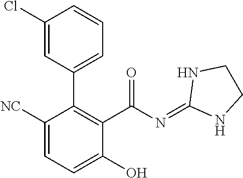 [SMILES: N#Cc1ccc(O)c(C(=O)N=C2NCCN2)c1-c1cccc(Cl)c1] | 3′-chloro-6-cyano-3-hydroxy-N-(imidazolidin-2- ylidene)-[1,1′-biphenyl]-2-carboxamide LC-MS: m/z 341 (M + H)⁺. 1H NMR (400 MHz, DMSO) δ: 8.55 (s, 2H), 7.68 (d, J = 8.7 Hz, 1H), 7.43-7.34 (m, 2H), 7.25 (s, 1H), 7.16-7.11 (m, 1H), 6.95 (d, J = 8.7 Hz, 1H), 3.53 (s, 4H). |
| 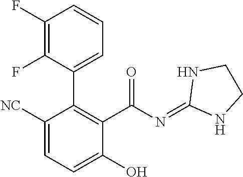 [SMILES: N#Cc1ccc(O)c(C(=O)N=C2NCCN2)c1-c1cccc(F)c1F] | 6-cyano-2′,3′-difluoro-3-hydroxy-N- (imidazolidin-2-ylidene)-[1,1′-biphenyl]-2- carboxamide LC-MS: m/z 343 (M + H)⁺. 1H NMR (400 MHz, DMSO) δ: 8.58 (s, 2H), 7.74 (d, J = 8.7 Hz, 1H), 7.45-7.31 (m, 1H), 7.21 (dd, J = 12.9, 8.0 Hz, 1H), 7.09-6.97 (m, 2H), 3.54 (s, 4H). |
| 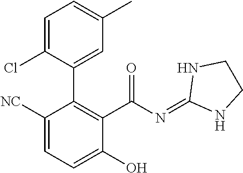 [SMILES: Cc1ccc(Cl)c(-c2c(C#N)ccc(O)c2C(=O)N=C2NCCN2)c1] | 2′-chloro-6-cyano-3-hydroxy-N-(imidazolidin-2- ylidene)-5′-methyl-[1,1′-biphenyl]-2- carboxamide LC-MS: m/z 355 (M + H)⁺. 1H NMR (400 MHz, DMSO) δ 8.52 (s, 2H), 7.69 (d, J = 8.4 Hz, 1H), 7.33 (d, J = 8.0 Hz, 1H), 7.14 (dd, J = 8.0, 1.6 Hz, 1H), 7.01 (d, J = 2.0 Hz, 1H), 6.95 (d, J = 8.8 Hz, 1H), 3.52 (s, 4H), 2.29 (s, 3H). |
| 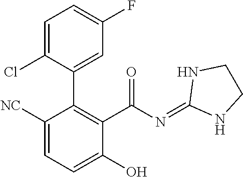 [SMILES: N#Cc1ccc(O)c(C(=O)N=C2NCCN2)c1-c1cc(F)ccc1Cl] | 2′-chloro-6-cyano-5′-fluoro-3-hydroxy-N- (imidazolidin-2-ylidene)-[1,1′-biphenyl]-2- carboxamide LC-MS: m/z 359 (M + H)⁺. 1H NMR (400 MHz, DMSO) δ 8.55 (s, 2H), 7.73 (d, J = 8.7 Hz, 1H), 7.51 (dd, J = 8.7, 5.1 Hz, 1H), 7.24-7.17 (m, 2H), 6.98 (d, J = 8.7 Hz, 1H), 3.54 (s, 4H). |
| 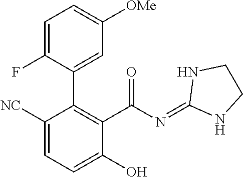 [SMILES: COc1ccc(F)c(-c2c(C#N)ccc(O)c2C(=O)N=C2NCCN2)c1] | 6-cyano-2′-fluoro-3-hydroxy-N-(imidazolidin-2- ylidene)-5′-methoxy-[1,1′-biphenyl]-2- carboxamide LC-MS: m/z 355 (M + H)⁺. 1H NMR (400 MHz, DMSO) δ 8.56 (s, 2H), 7.69 (d, J = 8.7 Hz, 1H), 7.09 (t, J = 9.1 Hz, 1H), 6.95 (d, J = 8.7 Hz, 1H), 6.88 (dt, J = 9.0, 3.6 Hz, 1H), 6.74 (dd, J = 6.0, 3.1 Hz, 1H), 3.72 (s, 3H), 3.52 (s, 4H). |
| 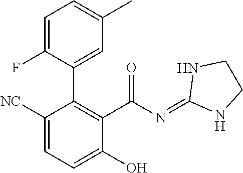 [SMILES: Cc1ccc(F)c(-c2c(C#N)ccc(O)c2C(=O)N=C2NCCN2)c1] | 6-cyano-2′-fluoro-3-hydroxy-N-(imidazolidin-2- ylidene)-5′-methyl-[1,1′-biphenyl]-2- carboxamide LC-MS: m/z 339 (M + H)⁺. 1H NMR (400 MHz, DMSO) δ 8.56 (s, 2H), 7.70 (d, J = 8.7 Hz, 1H), 7.17-7.12 (m, 1H), 7.09-7.02 (m, 1H), 7.01-6.93 (m, 2H), 3.53 (s, 4H), 2.28 (s, 3H). |
| 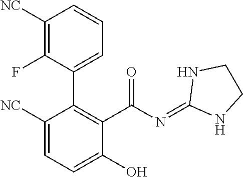 [SMILES: N#Cc1cccc(-c2c(C#N)ccc(O)c2C(=O)N=C2NCCN2)c1F] | 3′,6-dicyano-2′-fluoro-3-hydroxy-N- (imidazolidin-2-ylidene)-[1,1′-biphenyl]-2- carboxamide LC-MS: m/z 345 (M + H)⁺. 1H NMR (400 MHz, DMSO) δ: 8.52 (s, 2H), 7.97-7.89 (m, 1H), 7.76 (d, J = 8.7 Hz, 1H), 7.65 (td, J = 7.5, 1.6 Hz, 1H), 7.44 (t, J = 7.7 Hz, 1H), 7.01 (d, J = 8.7 Hz, 1H), 3.53 (s, 4H). |
| 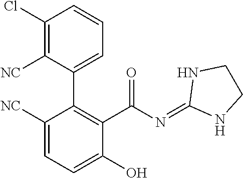 [SMILES: N#Cc1ccc(O)c(C(=O)N=C2NCCN2)c1-c1cccc(Cl)c1C#N] | 3′-chloro-2′,6-dicyano-3-hydroxy-N- (imidazolidin-2-ylidene)-[1,1′-biphenyl]-2- carboxamide LC-MS: m/z 366 (M + H)⁺. 1H NMR (400 MHz, DMSO) δ: 8.60 (s, 2H), 7.79 (d, J = 8.7 Hz, 1H), 7.76-7.70 (m, 2H), 7.39 (dd, J = 6.6, 2.2 Hz, 1H), 7.04 (d, J = 8.7 Hz, 1H), 3.54 (s, 4H). |
| 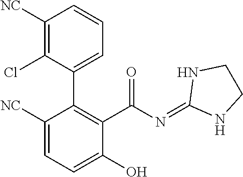 [SMILES: N#Cc1cccc(-c2c(C#N)ccc(O)c2C(=O)N=C2NCCN2)c1Cl] | 2′-chloro-3′,6-dicyano-3-hydroxy-N- (imidazolidin-2-ylidene)-[1,1′-biphenyl]-2- carboxamide LC-MS: m/z 366 (M + H)⁺. 1H NMR (400 MHz, DMSO) δ: 7.96 (dd, J = 7.3, 2.1 Hz, 1H), 7.76 (d, J = 8.7 Hz, 1H), 7.63- 7.53 (m, 2H), 7.00 (d, J = 8.7 Hz, 1H), 3.52 (s, 4H). |
| 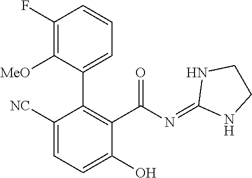 [SMILES: COc1c(F)cccc1-c1c(C#N)ccc(O)c1C(=O)N=C1NCCN1] | 6-cyano-3′-fluoro-3-hydroxy-N-(imidazolidin-2- ylidene)-2′-methoxy-[1,1′-biphenyl]-2- carboxamide LC-MS: m/z 355 (M + H)⁺. 1H NMR (400 MHz, DMSO) δ 7.68 (d, J = 8.7 Hz, 1H), 7.21 (ddd, J = 11.9, 8.3, 1.5 Hz, 1H), 7.08 (td, J = 8.0, 5.1 Hz, 1H), 6.94 (d, J = 8.7 Hz, 1H), 6.90-6.82 (m, 1H), 3.64 (s, 3H), 3.53 (s, 4H). |
| 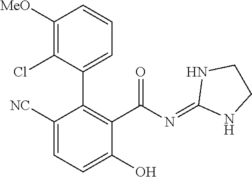 [SMILES: COc1cccc(-c2c(C#N)ccc(O)c2C(=O)N=C2NCCN2)c1Cl] | 2′-chloro-6-cyano-3-hydroxy-N-(imidazolidin-2- ylidene)-3′-methoxy-[1,1′-biphenyl]-2- carboxamide LC-MS: m/z 371 (M + H)⁺. 1H NMR (400 MHz, DMSO) δ: 8.55 (s, 2H), 7.69 (d, J = 8.7 Hz, 1H), 7.29 (t, J = 8.0 Hz, 1H), 7.08 (dd, J = 8.4, 1.2 Hz, 1H), 6.95 (d, J = 8.7 Hz, 1H), 6.77 (dd, J = 7.6, 1.3 Hz, 1H), 3.88 (s, 3H), 3.52 (s, 4H). |
| 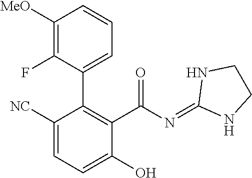 [SMILES: COc1cccc(-c2c(C#N)ccc(O)c2C(=O)N=C2NCCN2)c1F] | 6-cyano-2′-fluoro-3-hydroxy-N-(imidazolidin-2- ylidene)-3′-methoxy-[1,1′-biphenyl]-2- carboxamide LC-MS: m/z 355 (M + H)⁺. 1H NMR (400 MHz, DMSO) δ 8.55 (br s, 2H), 7.70 (d, J = 8.7 Hz, 1H), 7.15-7.08 (m, 2H), 6.96 (d, J = 8.7 Hz, 1H), 6.76-6.69 (m, 1H), 3.86 (s, 3H), 3.53 (s, 4H). |
| 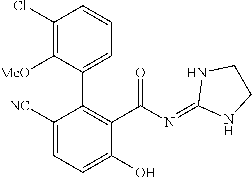 [SMILES: COc1c(Cl)cccc1-c1c(C#N)ccc(O)c1C(=O)N=C1NCCN1] | 3′-chloro-6-cyano-3-hydroxy-N-(imidazolidin-2- ylidene)-2′-methoxy-[1,1′-biphenyl]-2- carboxamide LC-MS: m/z 371 (M + H)⁺. 1H NMR (400 MHz, DMSO) δ 8.27 (br s, 2H), 7.70 (d, J = 8.7 Hz, 1H), 7.43 (dd, J = 8.0, 1.6 Hz, 1H), 7.13 (t, J = 7.8 Hz, 1H), 7.04 (dd, J = 7.6, 1.6 Hz, 1H), 6.96 (d, J = 8.7 Hz, 1H), 3.52 (s, 4H), 3.50 (s, 3H). |
| 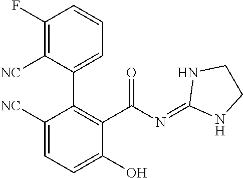 [SMILES: N#Cc1ccc(O)c(C(=O)N=C2NCCN2)c1-c1cccc(F)c1C#N] | 2′,6-dicyano-3′-fluoro-3-hydroxy-N- (imidazolidin-2-ylidene)-[1,1′-biphenyl]-2- carboxamide LC-MS: m/z 350 (M + H)⁺. 1H NMR (400 MHz, DMSO) δ: 8.59 (s, 2H), 7.84-7.76 (m, 2H), 7.52 (t, J = 9.0 Hz, 1H), 7.27 (d, J = 8.7 Hz, 1H), 7.05 (d, J = 8.7 Hz, 1H), 3.54 (s, 4H). |
| 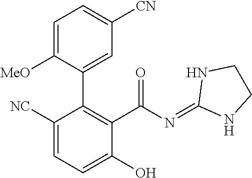 [SMILES: COc1ccc(C#N)cc1-c1c(C#N)ccc(O)c1C(=O)N=C1NCCN1] | 5′,6-dicyano-3-hydroxy-N-(imidazolidin-2- ylidene)-2′-methoxy-[1,1′-biphenyl]-2- carboxamide LC-MS: m/z 362 (M + H)⁺. 1H NMR (400 MHz, DMSO) δ 8.52 (s, 2H), 7.81 (dd, J = 8.6, 2.1 Hz, 1H), 7.68 (d, J = 8.7 Hz, 1H), 7.50 (d, J = 2.1 Hz, 1H), 7.22 (d, J = 8.7 Hz, 1H), 6.94 (d, J = 8.7 Hz, 1H), 3.75 (s, 3H), 3.52 (s, 4H). |
| 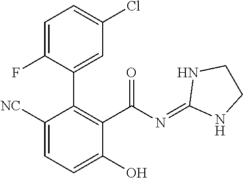 [SMILES: N#Cc1ccc(O)c(C(=O)N=C2NCCN2)c1-c1cc(Cl)ccc1F] | 5′-chloro-6-cyano-2′-fluoro-3-hydroxy-N- (imidazolidin-2-ylidene)-[1,1′-biphenyl]-2- carboxamide LC-MS: m/z 359 (M + H)⁺. 1H NMR (400 MHz, DMSO) δ 8.58 (s, 2H), 7.72 (d, J = 8.7 Hz, 1H), 7.42 (ddd, J = 8.7, 4.4, 2.7 Hz, 1H), 7.33 (dd, J = 6.3, 2.7 Hz, 1H), 7.26 (t, J = 9.0 Hz, 1H), 6.98 (d, J = 8.7 Hz, 1H), 3.53 (s, 4H). |
| 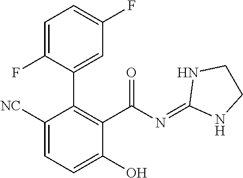 [SMILES: N#Cc1ccc(O)c(C(=O)N=C2NCCN2)c1-c1cc(F)ccc1F] | 6-cyano-2′,5′-difluoro-3-hydroxy-N- (imidazolidin-2-ylidene)-[1,1′-biphenyl]-2- carboxamide LC-MS: m/z 343 (M + H)⁺. 1H NMR (400 MHz, DMSO) δ 8.59 (s, 2H), 7.73 (d, J = 8.7 Hz, 1H), 7.29-7.12 (m, 3H), 6.99 (d, J = 8.7 Hz, 1H), 3.54 (s, 4H). |
| 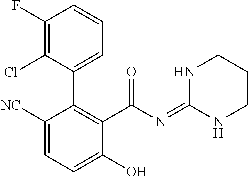 [SMILES: N#Cc1ccc(O)c(C(=O)N=C2NCCCN2)c1-c1cccc(F)c1Cl] | 2′-chloro-6-cyano-3′-fluoro-3-hydroxy-N- (tetrahydropyrimidin-2(1H)-ylidene)-[1,1′- biphenyl]-2-carboxamide LC-MS: m/z 373 (M + H)⁺. 1H NMR (400 MHz, DMSO) δ 8.91 (s, 2H), 7.67 (d, J = 8.7 Hz, 1H), 7.40-7.31 (m, 2H), 7.07-7.02 (m, 1H), 6.91 (d, J = 8.7 Hz, 1H), 3.25 (t, J = 5.7 Hz, 4H), 1.82-1.74 (m, 2H). |
| 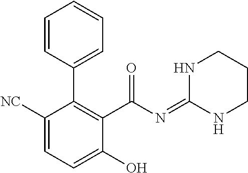 [SMILES: N#Cc1ccc(O)c(C(=O)N=C2NCCCN2)c1-c1ccccc1] | 6-cyano-3-hydroxy-N-(tetrahydropyrimidin- 2(1H)-ylidene)-[1,1′-biphenyl]-2-carboxamide LC-MS: m/z 321 (M + H)⁺. 1H NMR (400 MHz, DMSO) δ 8.90 (s, 2H), 7.61 (d, J = 8.7 Hz, 1H), 7.38-7.27 (m, 3H), 7.16 (d, J = 6.7 Hz, 2H), 6.87 (d, J = 8.7 Hz, 1H), 3.24 (t, J = 5.7 Hz, 4H), 1.84-1.73 (m, 2H). |
| 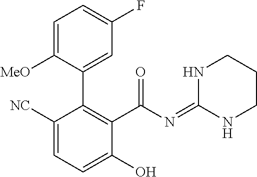 [SMILES: COc1ccc(F)cc1-c1c(C#N)ccc(O)c1C(=O)N=C1NCCCN1] | 6-cyano-5′-fluoro-3-hydroxy-2′-methoxy-N- (tetrahydropyrimidin-2(1H)-ylidene)-[1,1′- biphenyl]-2-carboxamide LC-MS: m/z 369 (M + H)⁺. 1H NMR (400 MHz, DMSO) δ 8.81 (br s, 2H), 7.60 (d, J = 8.7 Hz, 1H), 7.09 (td, J = 8.7, 3.1 Hz, 1H), 7.00 (dd, J = 9.1, 4.6 Hz, 1H), 6.86 (dd, J = 8.8, 1.9 Hz, 2H), 3.64 (s, 3H), 3.26 (t, J = 5.7 Hz, 4H), 1.78 (dd, J = 10.6, 5.7 Hz, 2H) |
| 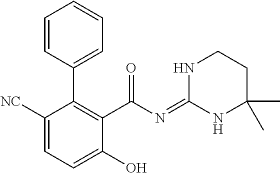 [SMILES: CC1(C)CCN/C(=N\C(=O)c2c(O)ccc(C#N)c2-c2ccccc2)N1] | (E)-6-cyano-N-(4,4- dimethyltetrahydropyrimidin-2(1H)-ylidene)-3- hydroxy-[1,1′-biphenyl]-2-carboxamide LC-MS: m/z 349 (M + H)⁺. 1H NMR (400 MHz, DMSO) δ 9.27 (s, 1H), 8.72 (s, 1H), 7.61 (d, J = 8.6 Hz, 1H), 7.38-7.29 (m, 3H), 7.15 (d, J = 6.5 Hz, 2H), 6.87 (d, J = 8.7 Hz, 1H), 2.50-2.47 (m, 2H), 1.67 (t, J = 5.9 Hz, 2H), 1.22 (s, 6H). |
| 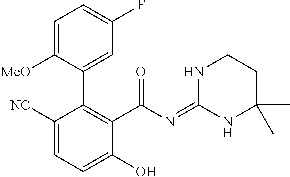 [SMILES: COc1ccc(F)cc1-c1c(C#N)ccc(O)c1C(=O)/N=C1\NCCC(C)(C)N1] | (E)-6-cyano-N-(4,4- dimethyltetrahydropyrimidin-2(1H)-ylidene)-5′- fluoro-3-hydroxy-2′-methoxy-[1,1′-biphenyl]-2- carboxamide LC-MS: m/z 397 (M + H)⁺. 1H NMR (400 MHz, DMSO) δ 9.29 (s, 1H), 8.71 (s, 1H), 7.60 (d, J = 8.8 Hz, 1H), 7.09 (td, J = 8.8, 3.2 Hz, 1H), 7.00 (dd, J = 9.2, 4.8 Hz, 1H), 6.85 (dd, J = 8.4, 2.0 Hz, 2H), 3.64 (s, 3H), 3.31- 3.27 (m, 2H), 1.67 (t, J = 6.0 Hz, 2H), 1.22 (s, 6H). |
| 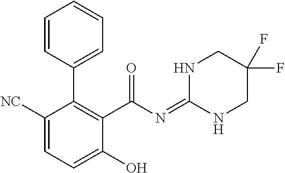 [SMILES: N#Cc1ccc(O)c(C(=O)N=C2NCC(F)(F)CN2)c1-c1ccccc1] | 6-cyano-N-(5,5-difluorotetrahydropyrimidin- 2(1H)-ylidene)-3-hydroxy-[1,1′-biphenyl]-2- carboxamide LC-MS: m/z 357 (M + H)⁺. 1H NMR (400 MHz, DMSO) δ 9.29 (s, 2H), 7.69 (d, J = 8.7 Hz, 1H), 7.39-7.29 (m, 3H), 7.21-7.16 (m, 2H), 6.96 (d, J = 8.6 Hz, 1H), 3.68 (t, J = 12.4 Hz, 4H). |
| 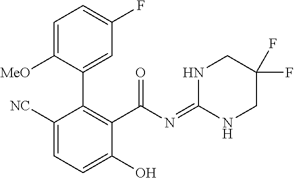 [SMILES: COc1ccc(F)cc1-c1c(C#N)ccc(O)c1C(=O)N=C1NCC(F)(F)CN1] | 6-cyano-N-(5,5-difluorotetrahydropyrimidin- 2(1H)-ylidene)-5′-fluoro-3-hydroxy-2′-methoxy- [1,1'-biphenyl]-2-carboxamide LC-MS: m/z 405 (M + H)⁺. 1H NMR (400 MHz, DMSO) δ 9.31 (s, 2H), 7.68 (d, J = 8.6 Hz, 1H), 7.11 (td, J = 8.7, 3.1 Hz, 1H), 7.02 (dd, J = 9.1, 4.6 Hz, 1H), 6.94 (d, J = 8.6 Hz, 1H), 6.90 (dd, J = 8.9, 3.1 Hz, 1H), 3.74- 3.66 (m, 4H), 3.65 (s, 3H). |
| 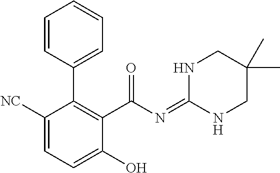 [SMILES: CC1(C)CNC(=NC(=O)c2c(O)ccc(C#N)c2-c2ccccc2)NC1] | 6-cyano-N-(5,5-dimethyltetrahydropyrimidin- 2(1H)-ylidene)-3-hydroxy-[1,1′-biphenyl]-2- carboxamide LC-MS: m/z 349 (M + H)⁺. 1H NMR (400 MHz, DMSO) δ 8.96 (br s, 2H), 7.61 (d, J = 8.7 Hz, 1H), 7.44-7.25 (m, 3H), 7.22-7.10 (m, 2H), 6.88 (d, J = 8.7 Hz, 1H), 2.95 (s, 4H), 0.94 (s, 6H). |
| 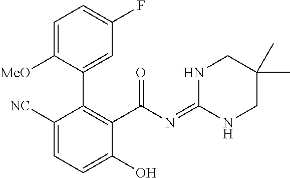 [SMILES: COc1ccc(F)cc1-c1c(C#N)ccc(O)c1C(=O)N=C1NCC(C)(C)CN1] | 6-cyano-N-(5,5-dimethyltetrahydropyrimidin- 2(1H)-ylidene)-5′-fluoro-3-hydroxy-2′-methoxy- [1,1′-biphenyl]-2-carboxamide LC-MS: m/z 397 (M + H)⁺. 1H NMR (400 MHz, DMSO) δ 8.95 (s, 2H), 7.60 (d, J = 8.7 Hz, 1H), 7.08 (td, J = 8.7, 3.1 Hz, 1H), 6.99 (dd, J = 9.1, 4.6 Hz, 1H), 6.89-6.81 (m, 2H), 3.63 (s, 3H), 2.95 (s, 4H), 0.93 (s, 6H). |
| 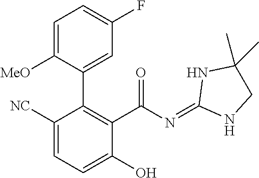 [SMILES: COc1ccc(F)cc1-c1c(C#N)ccc(O)c1C(=O)/N=C1/NCC(C)(C)N1] | (Z)-6-cyano-N-(4,4-dimethylimidazolidin-2- ylidene)-5′-fluoro-3-hydroxy-2′-methoxy-[1,1'- biphenyl]-2-carboxamide LC-MS: m/z 383 (M + H)⁺. 1H NMR (400 MHz, DMSO) δ 8.56 (br s, 2H), 7.70 (d, J = 8.8 Hz, 1H), 7.15 (td, J = 9.2, 3.2 Hz, 1H), 7.06 (dd, J = 9.2, 4.8 Hz, 1H), 7.00-6.90 (m, 2H), 3.69 (s, 3H), 3.33 (s, 2H), 1.31 (s, 6H). |
| 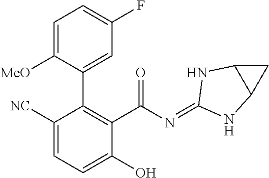 [SMILES: COc1ccc(F)cc1-c1c(C#N)ccc(O)c1C(=O)N=C1NC2CC2N1] | N-(2,4-diazabicyclo[3.1.0]hexan-3-ylidene)-6- cyano-5′-fluoro-3-hydroxy-2′-methoxy-[1,1′- biphenyl]-2-carboxamide LC-MS: m/z 367 (M + H)⁺. 1H NMR (400 MHz, DMSO) δ 9.26 (s, 2H), 7.65 (d, J = 8.6 Hz, 1H), 7.12-7.06 (m, 1H), 7.00 (dd, J = 9.1, 4.6 Hz, 1H), 6.93-6.84 (m, 2H), 3.62 (s, 3H), 3.46 (dd, J = 6.1, 2.4 Hz, 2H), 0.75-0.71 (m, 1H), 0.08-0.04 (m, 1H). |
| 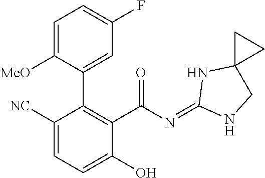 [SMILES: COc1ccc(F)cc1-c1c(C#N)ccc(O)c1C(=O)/N=C1/NCC2(CC2)N1] | (Z)-6-cyano-5′-fluoro-3-hydroxy-2′-methoxy-N- (4,6-diazaspiro[2.4]heptan-5-ylidene)-[1,1'- biphenyl]-2-carboxamide LC-MS: m/z 381 (M + H)⁺. 1H NMR (400 MHz, DMSO) δ 8.67 (s, 1H), 8.57 (s, 1H), 7.66 (d, J = 8.8 Hz, 1H), 7.09 (td, J = 8.8, 3.2 Hz, 1H), 7.00 (dd, J = 8.8, 4.4 Hz, 1H), 6.95-6.86 (m, 2H), 3.64 (s, 3H), 3.56 (s, 2H), 0.95 (q, J = 5.6 Hz, 2H), 0.69 (q, J = 5.6 Hz, 2H). |
| 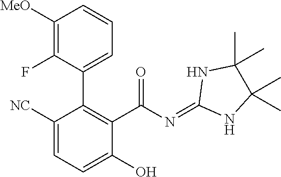 [SMILES: COc1cccc(-c2c(C#N)ccc(O)c2C(=O)N=C2NC(C)(C)C(C)(C)N2)c1F] | 6-cyano-2′-fluoro-3-hydroxy-3′-methoxy-N- (4,4,5,5-tetramethylimidazolidin-2-ylidene)- [1,1'-biphenyl]-2-carboxamide LC-MS: m/z 411 (M + H)⁺. 1H NMR (400 MHz, DMSO) δ 8.59 (s, 2H), 7.69 (d, J = 8.7 Hz, 1H), 7.15-7.08 (m, 2H), 6.95 (d, J = 8.7 Hz, 1H), 6.76-6.68 (m, 1H), 3.86 (s, 3H), 1.15 (s, 12H). |
| 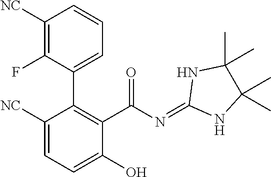 [SMILES: CC1(C)NC(=NC(=O)c2c(O)ccc(C#N)c2-c2cccc(C#N)c2F)NC1(C)C] | 3′,6-dicyano-2′-fluoro-3-hydroxy-N-(4,4,5,5- tetramethylimidazolidin-2-ylidene)-[1,1'- biphenyl]-2-carboxamide LC-MS: m/z 406 (M + H)⁺. 1H NMR (400 MHz, DMSO) δ 8.64 (s, 2H), 7.95-7.91 (m, 1H), 7.76 (d, J = 8.7 Hz, 1H), 7.65 (td, J = 7.6, 1.7 Hz, 1H), 7.46 (t, J = 7.7 Hz, 1H), 7.02 (d, J = 8.7 Hz, 1H), 1.15 (s, 12H). |
| 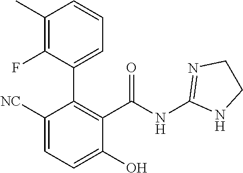 [SMILES: Cc1cccc(-c2c(C#N)ccc(O)c2C(=O)NC2=NCCN2)c1F] | 6-cyano-N-(4,5-dihydro-1H-imidazol-2-yl)-2′- fluoro-3-hydroxy-3′-methyl-[1,1′-biphenyl]-2- carboxamide LC-MS: m/z 339 (M + H)⁺. 1H NMR (400 MHz, DMSO-d6) δ 8.50 (br s, 2H), 7.68 (d, J = 8.7 Hz, 1H), 7.23 (t, J = 7.0 Hz, 1H), 7.07 (t, J = 7.5 Hz, 1H), 6.99 (t, J = 6.7 Hz, 1H), 6.94 (d, J = 8.7 Hz, 1H), 3.52 (s, 4H), 2.24 (s, 3H). |
| 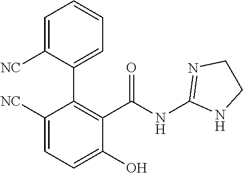 [SMILES: N#Cc1ccccc1-c1c(C#N)ccc(O)c1C(=O)NC1=NCCN1] | 2′,6-dicyano-N-(4,5-dihydro-1H-imidazol-2-yl)- 3-hydroxy-[1,1′-biphenyl]-2-carboxamide LC-MS: m/z 332 (M + H)⁺. 1H NMR (400 MHz, DMSO-d6) δ 8.50 (br s, 2H), 7.82-7.76 (m, 1H), 7.69 (d, J = 8.7 Hz, 1H), 7.64 (td, J = 7.7, 1.2 Hz, 1H), 7.46 (td, J = 7.7, 1.1 Hz, 1H), 7.31 (d, J = 7.4 Hz, 1H), 6.95 (d, J = 8.7 Hz, 1H), 3.46 (s, 4H). |
| 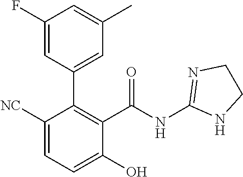 [SMILES: Cc1cc(F)cc(-c2c(C#N)ccc(O)c2C(=O)NC2=NCCN2)c1] | 6-cyano-N-(4,5-dihydro-1H-imidazol-2-yl)-3′- fluoro-3-hydroxy-5′-methyl-[1,1′-biphenyl]-2- carboxamide LC-MS: m/z 339 (M + H)⁺. 1H NMR (400 MHz, DMSO-d6) δ 7.62 (d, J = 8.7 Hz, 1H), 6.96 (d, J = 10.1 Hz, 1H), 6.89 (d, J = 8.7 Hz, 1H), 6.81 (s, 1H), 6.79 (d, J = 1.7 Hz, 1H), 3.51 (s, 4H), 2.33 (s, 3H). |
| 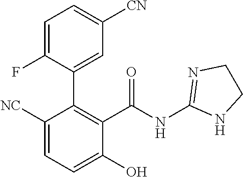 [SMILES: N#Cc1ccc(F)c(-c2c(C#N)ccc(O)c2C(=O)NC2=NCCN2)c1] | 5′,6-dicyano-N-(4,5-dihydro-1H-imidazol-2-yl)- 2′-fluoro-3-hydroxy-[1,1′-biphenyl]-2- carboxamide LC-MS: m/z 350 (M + H)⁺. 1H NMR (400 MHz, DMSO) δ 8.55 (brs, 2H), 7.99-7.91 (m, 1H), 7.88 (dd, J = 6.4, 2.0 Hz, 1H), 7.76 (d, J = 8.8 Hz, 1H), 7.49 (t, J = 9.2 Hz, 1H), 7.02 (d, J = 8.8 Hz, 1H), 3.54 (s, 4H). |
| 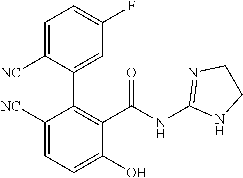 [SMILES: N#Cc1ccc(F)cc1-c1c(C#N)ccc(O)c1C(=O)NC1=NCCN1] | 2′,6-dicyano-N-(4,5-dihydro-1H-imidazol-2-yl)- 5′-fluoro-3-hydroxy-[1,1′-biphenyl]-2- carboxamide LC-MS: m/z 350 (M + H)⁺. 1H NMR (400 MHz, DMSO) δ 8.60 (brs, 2H), 8.03-7.94 (m, 1H), 7.78 (d, J = 8.7 0 Hz, 1H), 7.43-7.39 (m, 2H), 7.03 (d, J = 8.7 0 Hz, 1H), 3.54 (s, 4H). |

### General Procedure 10

Combination of the general procedures 5 and 9 described above.

The compounds in the table below were prepared from the appropriate starting materials described previously or commercially available using above general procedure 10

| Structure | Name |
|---|---|
| 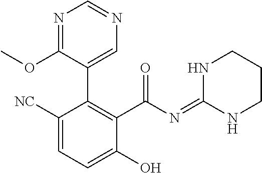 [SMILES: COc1ncncc1-c1c(C#N)ccc(O)c1C(=O)N=C1NCCCN1] | 3-cyano-6-hydroxy-2-(4-methoxypyrimidin-5- yl)-N-(tetrahydropyrimidin-2(1H)- ylidene)benzamide LC-MS: m/z 353 (M + H)⁺. 1H NMR (400 MHz, DMSO) δ 8.92 (s, 2H), 8.77 (s, 1H), 8.26 (s, 1H), 7.68 (d, J = 8.8 Hz, 1H), 6.92 (d, J = 8.4 Hz, 1H), 3.86 (s, 3H), 3.26 (t, J = 6.0 Hz, 4H), 1.83-1.73 (m, 2H). |
| 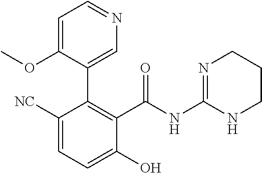 [SMILES: COc1ccncc1-c1c(C#N)ccc(O)c1C(=O)NC1=NCCCN1] | 3-cyano-6-hydroxy-2-(4-methoxypyridin-3-yl)- N-(1,4,5,6-tetrahydropyrimidin-2-yl)benzamide LC-MS: m/z 352 (M + H)⁺. 1H NMR (400 MHz, DMSO) δ 8.89 (s, 2H), 8.40 (d, J = 6.0 Hz, 1H), 8.02 (s, 1H), 7.63 (d, J = 8.4 Hz, 1H), 7.07 (d, J = 6.0 Hz, 1H), 6.88 (d, J = 8.8 Hz, 1H), 3.75 (s, 3H), 3.25 (t, J = 5.6 Hz, 4H), 1.83-1.74 (m, 2H). |
| 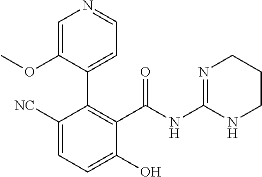 [SMILES: COc1cnccc1-c1c(C#N)ccc(O)c1C(=O)NC1=NCCCN1] | 3-cyano-6-hydroxy-2-(3-methoxypyridin-4-yl)- N-(1,4,5,6-tetrahydropyrimidin-2-yl)benzamide LC-MS: m/z 352 (M + H)⁺. 1H NMR (400 MHz, DMSO) δ 8.89 (s, 2H), 8.37 (s, 1H), 8.19 (d, J = 4.7 Hz, 1H), 7.63 (d, J = 8.7 Hz, 1H), 7.04 (d, J = 4.7 Hz, 1H), 6.88 (d, J = 8.7 Hz, 1H), 3.78 (s, 3H), 3.24 (t, J = 5.7 Hz, 4H), 1.80-1.75 (m, 2H). |
| 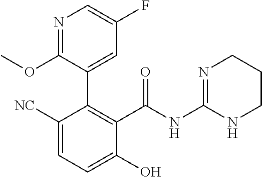 [SMILES: COc1ncc(F)cc1-c1c(C#N)ccc(O)c1C(=O)NC1=NCCCN1] | 3-cyano-2-(5-fluoro-2-methoxypyridin-3-yl)-6- hydroxy-N-(1,4,5,6-tetrahydropyrimidin-2- yl)benzamide LC-MS: m/z 370 (M + H)⁺. 1H NMR (400 MHz, DMSO) δ 8.90 (s, 2H), 8.09 (d, J = 3.2 Hz, 1H), 7.64 (d, J = 8.8 Hz, 1H), 7.51 (dd, J = 8.4, 2.8 Hz, 1H), 6.88 (d, J = 8.8 Hz, 1H), 3.74 (s, 3H), 3.25 (t, J = 5.6 Hz, 4H), 1.85-1.73 (m, 2H). |
| 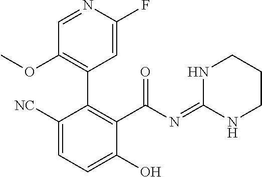 [SMILES: COc1cnc(F)cc1-c1c(C#N)ccc(O)c1C(=O)N=C1NCCCN1] | 3-cyano-2-(2-fluoro-5-methoxypyridin-4-yl)-6- hydroxy-N-(tetrahydropyrimidin-2(1H)- ylidene)benzamide LC-MS: m/z 370 (M + H)⁺. 1H NMR (400 MHz, DMSO) δ 8.90 (s, 2H), 7.92 (d, J = 1.4 Hz, 1H), 7.66 (d, J = 8.7 Hz, 1H), 6.97 (d, J = 2.9 Hz, 1H), 6.91 (d, J = 8.7 Hz, 1H), 3.76 (s, 3H), 3.26 (t, J = 5.7 Hz, 4H), 1.84-1.74 (m, 2H). |
| 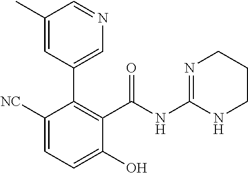 [SMILES: Cc1cncc(-c2c(C#N)ccc(O)c2C(=O)NC2=NCCCN2)c1] | 3-cyano-6-hydroxy-2-(5-methylpyridin-3-yl)-N- (1,4,5,6-tetrahydropyrimidin-2-yl)benzamide LC-MS: m/z 336 (M + H)⁺. 1H NMR (400 MHz, DMSO) δ 8.65 (br s, 2H), 8.40 (d, J = 1.6 Hz, 1H), 8.19 (d, J = 2.0 Hz, 1H), 7.68 (d, J = 8.8 Hz, 1H), 7.48 (s, 1H), 6.93 (d, J = 8.4 Hz, 1H), 3.30 (t, J = 5.6 Hz, 4H), 2.38 (s, 3H), 1.89-1.76 (m, 2H). |
| 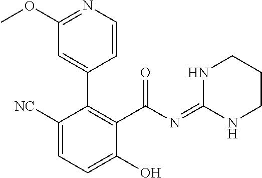 [SMILES: COc1cc(-c2c(C#N)ccc(O)c2C(=O)N=C2NCCCN2)ccn1] | 3-cyano-6-hydroxy-2-(2-methoxypyridin-4-yl)- N-(tetrahydropyrimidin-2(1H)- ylidene)benzamide LC-MS: m/z 352 (M + H)⁺. 1H NMR (400 MHz, DMSO) δ 8.93 (s, 2H), 8.12 (d, J = 5.3 Hz, 1H), 7.63 (d, J = 8.7 Hz, 1H), 6.89 (d, J = 8.7 Hz, 1H), 6.79 (dd, J = 5.2, 1.4 Hz, 1H), 6.61 (d, J = 0.5 Hz, 1H), 3.88 (s, 3H), 3.25 (t, J = 5.7 Hz, 4H), 1.84-1.72 (m, 2H). |
| 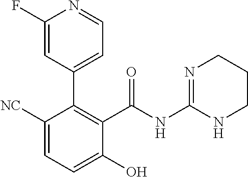 [SMILES: N#Cc1ccc(O)c(C(=O)NC2=NCCCN2)c1-c1ccnc(F)c1] | 3-cyano-2-(2-fluoropyridin-4-yl)-6-hydroxy-N- (1,4,5,6-tetrahydropyrimidin-2-yl)benzamide LC-MS: m/z 340 (M + H)⁺. 1H NMR (400 MHz, DMSO) δ 8.89 (br s, 2H), 8.20 (d, J = 5.2 Hz, 1H), 7.64 (d, J = 8.8 Hz, 1H), 7.23-7.13 (m, 1H), 7.07 (s, 1H), 6.89 (d, J = 8.8 Hz, 1H), 3.24 (t, J = 5.6 Hz, 4H), 1.84- 1.70 (m, 2H). |
| 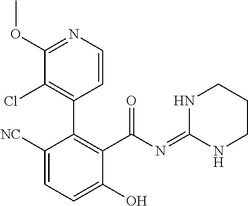 [SMILES: COc1nccc(-c2c(C#N)ccc(O)c2C(=O)N=C2NCCCN2)c1Cl] | 2-(3-chloro-2-methoxypyridin-4-yl)-3-cyano-6- hydroxy-N-(tetrahydropyrimidin-2(1H)- ylidene)benzamide LC-MS: m/z 386 (M + H)⁺. 1H NMR (400 MHz, DMSO-d6) δ: 8.92 (s, 2H), 8.10 (d, J = 5.1 Hz, 1H), 7.71 (d, J = 8.7 Hz, 1H), 6.94 (d, J = 8.5 Hz, 1H), 6.89 (d, J = 5.1 Hz 1H), 3.97 (s, 3H), 3.26 (t, J = 5.7 Hz, 4H), 1.83-1.78 (m, 2H). |
| 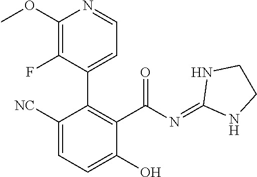 [SMILES: COc1nccc(-c2c(C#N)ccc(O)c2C(=O)N=C2NCCN2)c1F] | 3-cyano-2-(3-fluoro-2-methoxypyridin-4-yl)-6- hydroxy-N-(imidazolidin-2-ylidene)benzamide LC-MS: m/z 356 (M + H)⁺. 1H NMR (400 MHz, DMSO) δ 8.63 (s, 2H), 7.96 (d, J = 5.1 Hz, 1H), 7.76 (d, J = 8.7 Hz, 1H), 7.01 (d, J = 8.7 Hz, 1H), 6.89 (t, J = 4.8 Hz, 1H), 3.97 (s, 3H), 3.54 (s, 4H). |
| 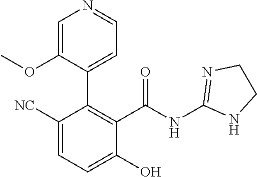 [SMILES: COc1cnccc1-c1c(C#N)ccc(O)c1C(=O)NC1=NCCN1] | 3-cyano-N-(4,5-dihydro-1H-imidazol-2-yl)-6- hydroxy-2-(3-methoxypyridin-4-yl)benzamide LC-MS: m/z 338 (M + H)⁺. 1H NMR (400 MHz, DMSO) δ 8.54 (s, 2H), 8.38 (s, 1H), 8.20 (d, J = 4.7 Hz, 1H), 7.69 (d, J = 8.7 Hz, 1H), 7.07 (d, J = 4.7 Hz, 1H), 6.94 (d, J = 8.7 Hz, 1H), 3.77 (s, 3H), 3.52 (s, 4H). |
| 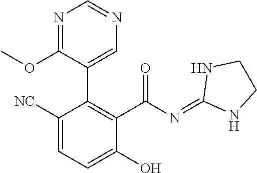 [SMILES: COc1ncncc1-c1c(C#N)ccc(O)c1C(=O)N=C1NCCN1] | 3-cyano-6-hydroxy-N-(imidazolidin-2-ylidene)- 2-(4-methoxypyrimidin-5-yl)benzamide LC-MS: m/z 339 (M + H)⁺. 1H NMR (400 MHz, DMSO) δ 8.78 (s, 1H), 8.54 (br s, 2H), 8.28 (s, 1H), 7.73 (d, J = 8.8 Hz, 1H), 6.97 (d, J = 8.8 Hz, 1H), 3.85 (s, 3H), 3.53 (s, 4H). |
| 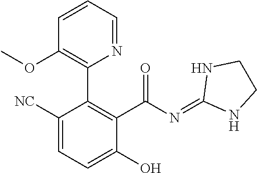 [SMILES: COc1cccnc1-c1c(C#N)ccc(O)c1C(=O)N=C1NCCN1] | 3-cyano-6-hydroxy-N-(imidazolidin-2-ylidene)- 2-(3-methoxypyridin-2-yl)benzamide LC-MS: m/z 338 (M + H)⁺. 1H NMR (400 MHz, DMSO) δ 8.45 (s, 2H), 8.10 (dd, J = 4.7, 1.2 Hz, 1H), 7.67 (d, J = 8.6 Hz, 1H), 7.42 (dd, J = 8.4, 1.2 Hz, 1H), 7.31 (dd, J = 8.3, 4.7 Hz, 1H),6.94 (d, J = 8.6 Hz, 1H), 3.70 (s, 3H), 3.52 (s, 4H). |
| 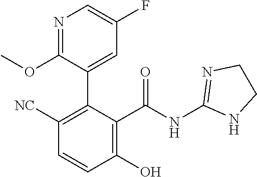 [SMILES: COc1ncc(F)cc1-c1c(C#N)ccc(O)c1C(=O)NC1=NCCN1] | 3-cyano-N-(4,5-dihydro-1H-imidazol-2-yl)-2- (5-fluoro-2-methoxypyridin-3-yl)-6- hydroxybenzamide LC-MS: m/z 356 (M + H)⁺. 1H NMR (400 MHz, DMSO) δ 8.27 (br s, 1H), 8.09 (d, J = 2.8 Hz, 1H), 7.65 (d, J = 8.8 Hz, 1H), 7.53 (dd, J = 8.4, 3.2 Hz, 1H), 6.90 (d, J = 8.8 Hz, 1H), 3.74 (s, 3H), 3.50 (s, 4H). |
| 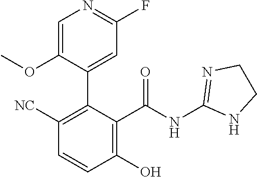 [SMILES: COc1cnc(F)cc1-c1c(C#N)ccc(O)c1C(=O)NC1=NCCN1] | 3-cyano-N-(4,5-dihydro-1H-imidazol-2-yl)-2- (2-fluoro-5-methoxypyridin-4-yl)-6- hydroxybenzamide LC-MS: m/z 356 (M + H)⁺. 1H NMR (400 MHz, DMSO) δ 8.48 (br s, 2H), 7.92 (d, J = 1.5 Hz, 1H), 7.69 (d, J = 8.7 Hz, 1H), 6.98 (d, J = 2.9 Hz, 1H), 6.93 (d, J = 8.7 Hz, 1H), 3.75 (s, 3H), 3.51 (s, 4H). |
| 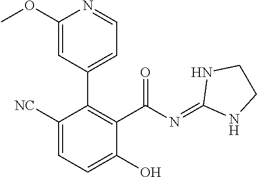 [SMILES: COc1cc(-c2c(C#N)ccc(O)c2C(=O)N=C2NCCN2)ccn1] | 3-cyano-6-hydroxy-N-(imidazolidin-2-ylidene)- 2-(2-methoxypyridin-4-yl)benzamide LC-MS: m/z 338 (M + H)⁺. 1H NMR (400 MHz, DMSO) δ 8.58 (s, 2H), 8.13 (d, J = 5.2 Hz, 1H), 7.69 (d, J = 8.7 Hz, 1H), 6.95 (d, J = 8.7 Hz, 1H), 6.80 (dd, J = 5.2, 1.4 Hz, 1H), 6.63 (s, 1H), 3.87 (s, 3H), 3.52 (s, 4H). |
| 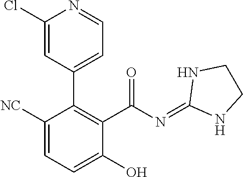 [SMILES: N#Cc1ccc(O)c(C(=O)N=C2NCCN2)c1-c1ccnc(Cl)c1] | 2-(2-chloropyridin-4-yl)-3-cyano-6-hydroxy-N- (imidazolidin-2-ylidene)benzamide LC-MS: m/z 342 (M + H)⁺. 1H NMR (400 MHz, DMSO) δ 8.56 (br s, 2H), 8.40 (d, J = 5.0 Hz, 1H), 7.72 (d, J = 8.7 Hz, 1H), 7.42 (s, 1H), 7.28 (d, J = 5.0 Hz, 1H), 6.97 (d, J = 8.7 Hz, 1H), 3.53 (s, 4H). |
| 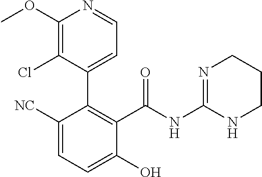 [SMILES: COc1nccc(-c2c(C#N)ccc(O)c2C(=O)NC2=NCCCN2)c1Cl] | 2-(3-chloro-2-methoxypyridin-4-yl)-3-cyano-6- hydroxy-N-(1,4,5,6-tetrahydropyrimidin-2- yl)benzamide LC-MS: m/z 386 (M + H)⁺. 1H NMR (400 MHz, DMSO) δ: 8.92 (s, 2H), 8.10 (d, J = 5.1 Hz, 1H), 7.71 (d, J = 8.7 Hz, 1H), 6.94 (d, J = 8.5 Hz, 1H), 6.89 (d, J = 5.1 Hz, 1H), 3.97 (s, 3H), 3.26 (t, J = 5.7 Hz, 4H), 1.83- 1.78 (m, 2H). |
| 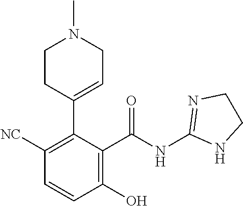 [SMILES: CN1CC=C(c2c(C#N)ccc(O)c2C(=O)NC2=NCCN2)CC1] | 3-cyano-N-(4,5-dihydro-1H-imidazol-2-yl)-6- hydroxy-2-(1-methyl-1,2,3,6-tetrahydropyridin- 4-yl)benzamide LC-MS: m/z 326 (M + H)⁺. 1H NMR (400 MHz, DMSO) δ 8.52 (br s, 2H), 7.57 (d, J = 8.8 Hz, 1H), 6.80 (d, J = 8.8 Hz, 1H), 5.33 (s, 1H), 3.58 (s, 4H), 2.94 (s, 2H), 2.61 (t, J = 5.6 Hz, 2H), 2.31-2.26 (m, 5H). |
| 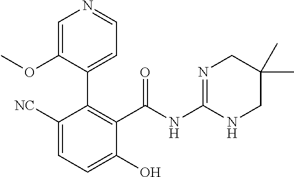 | 3-cyano-N-(5,5-dimethyl-1,4,5,6- tetrahydropyrimidin-2-yl)-6-hydroxy-2-(3- methoxypyridin-4-yl)benzamide LC-MS: m/z 380 (M + H)⁺. 1H NMR (400 MHz, DMSO) δ 8.37 (s, 1H), 8.20 (d, J = 4.7 Hz, 1H), 7.64 (d, J = 8.7 Hz, 1H), 7.06 (d, J = 4.7 Hz, 1H), 6.89 (d, J = 8.7 Hz, 1H), 3.78 (s, 3H), 2.95 (s, 4H), 0.93 (s, 6H). |
| 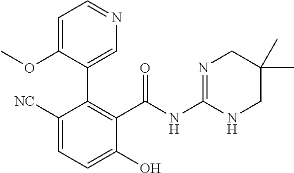 | 3-cyano-N-(5,5-dimethyl-1,4,5,6- tetrahydropyrimidin-2-yl)-6-hydroxy-2-(4- methoxypyridin-3-yl)benzamide LC-MS: m/z 380 (M + H)⁺. 1H NMR (400 MHz, DMSO) δ 8.97 (s, 2H), 8.41 (d, J = 5.7 Hz, 1H), 8.05 (s, 1H), 7.65 (d, J = 8.7 Hz, 1H), 7.09 (d, J = 5.8 Hz, 1H), 6.90 (d, J = 8.7 Hz, 1H), 3.75 (s, 3H), 2.96 (s, 4H), 0.94 (s, 6H). |
| 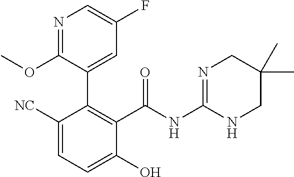 | 3-cyano-N-(5,5-dimethyl-1,4,5,6- tetrahydropyrimidin-2-yl)-2-(5-fluoro-2- methoxypyridin-3-yl)-6-hydroxybenzamide LC-MS: m/z 398 (M + H)⁺. 1H NMR (400 MHz, DMSO) δ 8.92 (br s, 2H), 8.09 (d, J = 2.8 Hz, 1H), 7.62 (d, J = 8.8 Hz, 1H), 7.52 (dd, J = 8.4, 2.8 Hz, 1H), 6.87 (d, J = 8.4 Hz, 1H), 3.74 (s, 3H), 2.95 (s, 4H), 0.93 (s, 6H). |
| 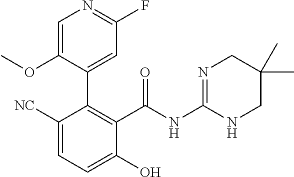 | 3-cyano-N-(5,5-dimethyl-1,4,5,6- tetrahydropyrimidin-2-yl)-2-(2-fluoro-5- methoxypyridin-4-yl)-6-hydroxybenzamide LC-MS: m/z 398 (M + H)⁺. 1H NMR (400 MHz, DMSO) δ 8.95 (s, 2H), 7.93 (d, J = 1.5 Hz, 1H), 7.68 (d, J = 8.7 Hz, 1H), 6.98 (d, J = 2.9 Hz, 1H), 6.92 (d, J = 8.7 Hz, 1H) 3.76 (s, 3H), 2.97 (s, 4H), 0.95 (s, 6H). |
| 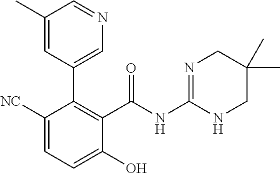 | 3-cyano-N-(5,5-dimethyl-1,4,5,6- tetrahydropyrimidin-2-yl)-6-hydroxy-2-(5- methylpyridin-3-yl)benzamide LC-MS: m/z 364 (M + H)⁺. 1H NMR (400 MHz, DMSO) δ 8.90 (br s, 2H), 8.35 (d, J = 1.6 Hz, 1H), 8.15 (d, J = 2.0 Hz, 1H), 7.64 (d, J = 8.8 Hz, 1H), 7.43 (s, 1H), 6.90 (d, J = 8.8 Hz, 1H), 2.95 (s, 4H), 2.32 (s, 3H), 0.94 (s, 6H). |
| 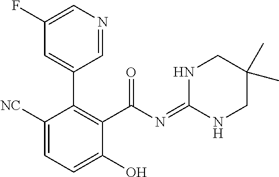 [SMILES: CC1(C)CNC(=NC(=O)c2c(O)ccc(C#N)c2-c2cncc(F)c2)NC1] | 3-cyano-N-(5,5-dimethyltetrahydropyrimidin- 2(1H)-ylidene)-2-(5-fluoropyridin-3-yl)-6- hydroxybenzamide LC-MS: m/z 368 (M + H)⁺. 1H NMR (400 MHz, DMSO) δ 8.96 (s, 2H), 8.52 (d, J = 2.7 Hz, 1H), 8.25 (t, J = 1.7 Hz, 1H), 7.70-7.65 (m, 2H), 6.93 (d, J = 8.7 Hz, 1H), 2.95 (s, 4H), 0.93 (s, 6H). |
| 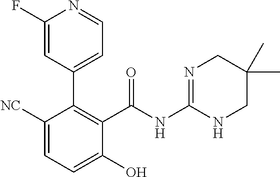 [SMILES: CC1(C)CN=C(NC(=O)c2c(O)ccc(C#N)c2-c2ccnc(F)c2)NC1] | 3-cyano-N-(5,5-dimethyl-1,4,5,6- tetrahydropyrimidin-2-yl)-2-(2-fluoropyridin-4- yl)-6-hydroxybenzamide LC-MS: m/z 368 (M + H)⁺. 1H NMR (400 MHz, DMSO) δ 8.92 (br s, 2H), 8.21 (d, J = 5.2 Hz, 1H), 7.65 (d, J = 8.8 Hz, 1H), 7.19 (d, J = 5.2 Hz, 1H), 7.08 (s, 1H), 6.90 (d, J = 8.8 Hz, 1H), 2.95 (s, 4H), 0.93 (s, 6H). |
| 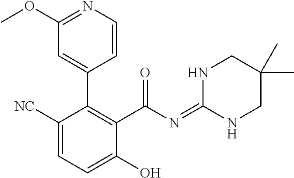 | 3-cyano-N-(5,5-dimethyltetrahydropyrimidin- 2(1H)-ylidene)-6-hydroxy-2-(2- methoxypyridin-4-yl)benzamide LC-MS: m/z 380 (M + H)⁺. 1H NMR (400 MHz, DMSO) δ 8.98 (s, 2H), 8.12 (d, J = 5.3 Hz, 1H), 7.64 (d, J = 8.7 Hz, 1H), 6.89 (d, J = 8.7 Hz, 1H), 6.79 (dd, J = 5.2, 1.4 Hz, 1H), 6.61 (d, J = 0.6 Hz, 1H), 3.87 (s, 3H), 2.95 (s, 4H), 0.94 (s, 6H). |
| 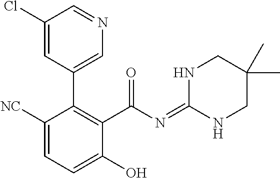 [SMILES: CC1(C)CNC(=NC(=O)c2c(O)ccc(C#N)c2-c2cncc(Cl)c2)NC1] | 2-(5-chloropyridin-3-yl)-3-cyano-N-(5,5- dimethyltetrahydropyrimidin-2(1H)-ylidene)-6- hydroxybenzamide LC-MS: m/z 384 (M + H)⁺. 1H NMR (400 MHz, DMSO) δ: 8.98 (s, 2H), 8.57 (d, J = 2.3 Hz, 1H), 8.33 (d, J = 1.8 Hz, 1H), 7.87-7.81 (m, 1H), 7.68 (d, J = 8.7 Hz, 1H), 6.94 (d, J = 8.7 Hz, 1H), 2.96 (s, 4H), 0.94 (s, 6H). |
| 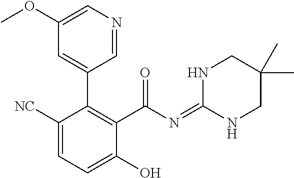 | 3-cyano-N-(5,5-dimethyltetrahydropyrimidin- 2(1H)-ylidene)-6-hydroxy-2-(5- methoxypyridin-3-yl)benzamide LC-MS: m/z 380 (M + H)⁺. 1H NMR (400 MHz, DMSO) δ 8.98 (s, 2H), 8.23 (d, J = 2.8 Hz, 1H), 7.95 (d, J = 1.7 Hz, 1H), 7.65 (d, J = 8.7 Hz, 1H), 7.23 (dd, J = 2.7, 1.8 Hz, 1H), 6.92 (d, J = 8.7 Hz, 1H), 3.83 (s, 3H), 2.95 (s, 4H), 0.94 (s, 6H). |
| 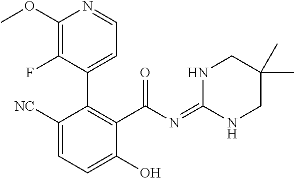 | 3-cyano-N-(5,5-dimethyltetrahydropyrimidin- 2(1H)-ylidene)-2-(3-fluoro-2-methoxypyridin- 4-yl)-6-hydroxybenzamide LC-MS: m/z 398 (M + H)⁺. 1H NMR (400 MHz, DMSO) δ 9.01 (s, 2H), 7.95 (d, J = 5.2 Hz, 1H), 7.71 (d, J = 8.8 Hz, 1H), 6.96 (d, J = 8.8 Hz, 1H), 6.88 (t, J = 4.8 Hz, 1H), 3.98 (s, 3H), 2.97 (s, 4H), 0.95 (s, 6H). |
| 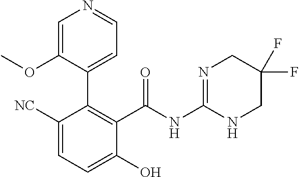 [SMILES: COc1cnccc1-c1c(C#N)ccc(O)c1C(=O)NC1=NCC(F)(F)CN1] | 3-cyano-N-(5,5-difluoro-1,4,5,6- tetrahydropyrimidin-2-yl)-6-hydroxy-2-(3- methoxypyridin-4-yl)benzamide LC-MS: m/z 388 (M + H)⁺. 1H NMR (400 MHz, DMSO) δ 9.29 (br s, 2H), 8.38 (s, 1H), 8.21 (d, J = 4.7 Hz, 1H), 7.72 (d, J = 8.7 Hz, 1H), 7.08 (d, J = 4.7 Hz, 1H), 6.98 (d, J = 8.7 Hz, 1H), 3.78 (s, 3H), 3.69 (t, J = 12.3 Hz, 4H). |
| 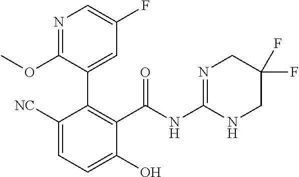 [SMILES: COc1ncc(F)cc1-c1c(C#N)ccc(O)c1C(=O)NC1=NCC(F)(F)CN1] | 3-cyano-N-(5,5-difluoro-1,4,5,6- tetrahydropyrimidin-2-yl)-2-(5-fluoro-2- methoxypyridin-3-yl)-6-hydroxybenzamide LC-MS: m/z 406 (M + H)⁺. 1H NMR (400 MHz, DMSO) δ 9.30 (s, 2H), 8.11 (d, J = 2.8 Hz, 1H), 7.72 (d, J = 8.8 Hz, 1H), 7.56 (dd, J = 8.4, 2.8 Hz, 1H), 6.97 (d, J = 8.4 Hz, 1H), 3.74 (s, 3H), 3.69 (t, J = 12.4 Hz, 4H). |
| 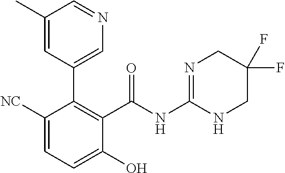 [SMILES: Cc1cncc(-c2c(C#N)ccc(O)c2C(=O)NC2=NCC(F)(F)CN2)c1] | 3-cyano-N-(5,5-difluoro-1,4,5,6- tetrahydropyrimidin-2-yl)-6-hydroxy-2-(5- methylpyridin-3-yl)benzamide LC-MS: m/z 372 (M + H)⁺. 1H NMR (400 MHz, DMSO) δ 9.28 (br s, 2H), 8.36 (s, 1H), 8.17 (d, J = 2.0 Hz, 1H), 7.73 (d, J = 8.4 Hz, 1H), 7.46 (s, 1H), 7.00 (d, J = 8.8 Hz, 1H), 3.69 (t, J = 12.4 Hz, 4H), 2.33 (s, 3H). |
| 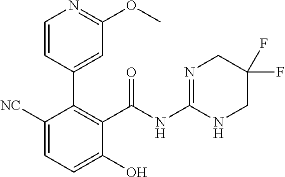 [SMILES: COc1cc(-c2c(C#N)ccc(O)c2C(=O)NC2=NCC(F)(F)CN2)ccn1] | 3-cyano-N-(5,5-difluoro-1,4,5,6- tetrahydropyrimidin-2-yl)-6-hydroxy-2-(2- methoxypyridin-4-yl)benzamide LC-MS: m/z 388 (M + H)⁺. 1H NMR (400 MHz, DMSO) δ 9.32 (s, 2H), 8.13 (d, J = 5.3 Hz, 1H), 7.71 (d, J = 8.7 Hz, 1H), 6.98 (d, J = 8.7 Hz, 1H), 6.81 (dd, J = 5.2, 1.4 Hz, 1H), 6.64 (s, 1H), 3.87 (s, 3H), 3.68 (t, J = 12.4 Hz, 4H). |

### General Procedure 11

Combination of the general procedures 5 and 8 described above.

The compounds in the table below were prepared from the appropriate starting materials described previously or commercially available using the above general procedure 11

| Structure | Name |
|---|---|
| 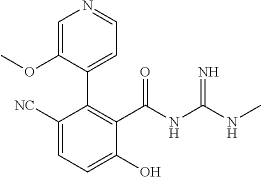 | 3-cyano-6-hydroxy-2-(3-methoxypyridin-4-yl)- N-(N-methylcarbamimidoyl)benzamide LC-MS: m/z 326 (M + H)⁺. 1H NMR (400 MHz, DMSO) δ 8.93 (br s, 1H), 8.37 (s, 1H), 8.20 (d, J = 4.7 Hz, 1H), 7.63 (d, J = 8.7 Hz, 1H), 7.06 (d, J = 4.5 Hz, 1H), 6.87 (d, J = 8.5 Hz, 1H), 3.78 (s, 3H), 2.73 (s, 3H). |
| 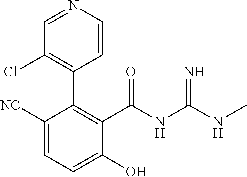 [SMILES: CNC(=N)NC(=O)c1c(O)ccc(C#N)c1-c1ccncc1Cl] | 2-(3-chloropyridin-4-yl)-3-cyano-6-hydroxy-N- (N-methylcarbamimidoyl)benzamide LC-MS: m/z 330 (M + H)⁺. 1H NMR (400 MHz, DMSO) δ 8.95 (d, J = 4.6 Hz, 1H), 8.65 (s, 1H), 8.52 (d, J = 4.9 Hz, 1H), 7.70 (d, J = 8.7 Hz, 1H), 7.32 (d, J = 4.9 Hz, 1H), 6.93 (d, J = 8.7 Hz, 1H), 2.74 (s, 3H). |
| 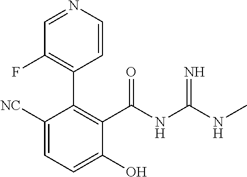 [SMILES: CNC(=N)NC(=O)c1c(O)ccc(C#N)c1-c1ccncc1F] | 3-cyano-2-(3-fluoropyridin-4-yl)-6-hydroxy-N- (N-methylcarbamimidoyl)benzamide LC-MS: m/z 314 (M + H)⁺. 1H NMR (400 MHz, DMSO) δ 9.02 (d, J = 3.9 Hz, 1H), 8.63 (s, 1H), 8.50 (d, J = 4.7 Hz, 1H), 7.77 (d, J = 8.7 Hz, 1H), 7.43-7.38 (m, 1H), 7.00 (d, J = 8.7 Hz, 1H), 2.80 (s, 3H). |
| 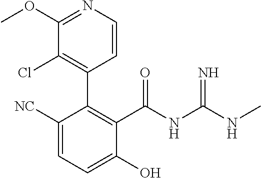 | 2-(3-chloro-2-methoxypyridin-4-yl)-3-cyano-6- hydroxy-N-(N-methylcarbamimidoyl)benzamide LC-MS: m/z 360 (M + H)⁺. 1H NMR (400 MHz, DMSO) δ: 8.95 (s, 1H), 8.46 (br s, 1H), 8.10 (d, J = 5.1 Hz, 1H), 7.68 (d, J = 8.7 Hz, 1H), 6.90 (t, J = 6.3 Hz, 2H), 3.97 (s, 3H), 2.74 (s, 3H). |
| 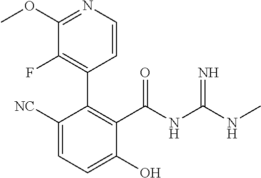 | 3-cyano-2-(3-fluoro-2-methoxypyridin-4-yl)-6- hydroxy-N-(N-methylcarbamimidoyl)benzamide LC-MS: m/z 344 (M + H)⁺. 1H NMR (400 MHz, DMSO) δ 7.96 (d, J = 5.1 Hz, 1H), 7.69 (d, J = 8.8 Hz, 1H), 6.93 (d, J = 8.8 Hz, 1H), 6.88 (t, J = 4.8 Hz, 1H), 3.98 (s, 3H), 2.75 (s, 3H). |
| 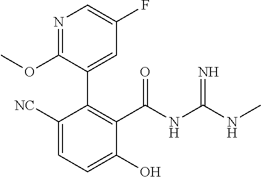 | 3-cyano-2-(5-fluoro-2-methoxypyridin-3-yl)-6- hydroxy-N-(N-methylcarbamimidoyl)benzamide LC-MS: m/z 344 (M + H)⁺. 1H NMR (400 MHz, DMSO) δ 8.20 (br s, 1H), 8.10 (d, J = 2.8 Hz, 1H), 7.64 (d, J = 8.8 Hz, 1H), 7.53 (dd, J = 8.4, 2.8 Hz, 1H), 6.87 (d, J = 8.4 Hz, 1H), 3.74 (s, 3H), 2.75 (s, 3H). |
| 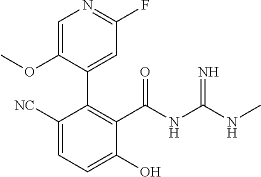 | 3-cyano-2-(2-fluoro-5-methoxypyridin-4-yl)-6- hydroxy-N-(N-methylcarbamimidoyl)benzamide LC-MS: m/z 344 (M + H)⁺. 1H NMR (400 MHz, DMSO) δ 8.21 (br s, 2H), 7.92 (d, J = 1.5 Hz, 1H), 7.65 (d, J = 8.7 Hz, 1H), 6.97 (d, J = 2.9 Hz, 1H), 6.88 (d, J = 8.7 Hz, 1H), 3.75 (s, 3H), 2.75 (s, 3H). |

### General Procedure 12

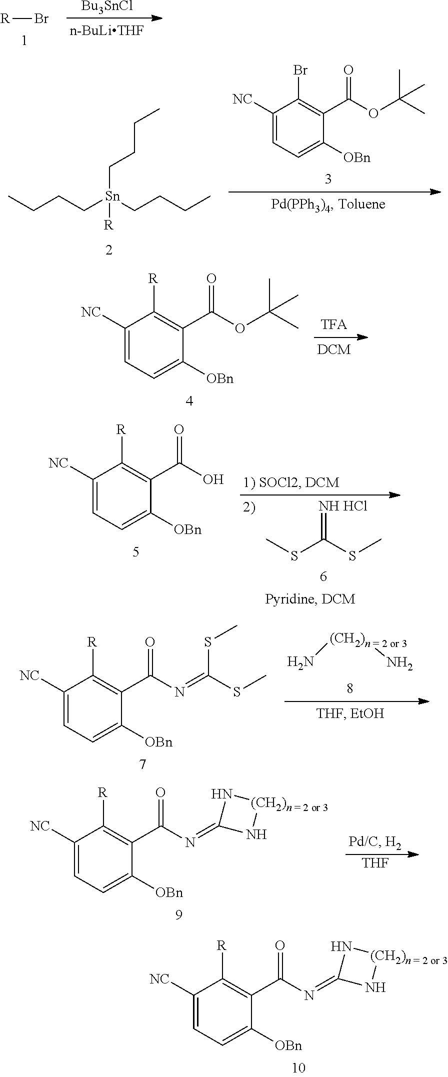

### Step 1: Synthesis of Compound 2

n-BuLi (1.1 eq.) was added dropwise at −20/−10° C. under N₂ atmosphere to a solution of compound 1 (1.0 eq.) in THF (0.1 mol/L), and the resulting mixture was stirred at −10° C. for 20 minutes before a solution of Bu₃SnCl (1.3 eq.) in anhydrous THF was added. The resulting mixture was then stirred at room temperature for 16 hours. The mixture was then quenched with saturated aq. NH₄Cl solution and extracted with EtOAc. The organic layer was separated, dried over Na₂SO₄, filtered and concentrated under vacuum. The residue was purified by column chromatography on silica gel (eluted with Petroleum Ether:EtOAc=100:0 to 25:1) to give compound 2.

### Step 2: Synthesis of Compound 4

Pd(PPh₃)₄ (0.1 eq.) was added under N₂ atmosphere to a suspension of 3 (1.0 eq.) and 2 (1.3 eq.) in toluene, and the resulting mixture was stirred at 100° C. for 20 hours. The mixture was then diluted with EtOAc and the organic layer was washed with water and brine, dried over Na₂SO₄, filtered and concentrated under vacuum. The residue was purified by column chromatography on silica gel (eluted with Petroleum Ether:EtOAc=10:1 to 5:1) to give compound 4.

### Step 3: Synthesis of Compound 5

TFA (10 eq.) was added under N₂ atmosphere to a solution of compound 4 (1 eq.) in DCM, and the resulting solution was stirred at room temperature for 3 hours. The reaction was then concentrated under vacuum and the residue was purified by column chromatography on silica gel (eluted with Petroleum Ether:EtOAc=10:1 to 1:1) to give compound 5.

### Step 4: Synthesis of Compound 7

Thionyl chloride (5 eq.) was added to a suspension of compound 5 (1 eq.) in anhydrous DCM, and the resulting mixture was stirred at 50° C. for 4-6 hours. After the reaction was completed, the mixture was concentrated under vacuum and the residue was then dissolved in anhydrous DCM and added to a mixture of compound 6 (1.5 eq.) and Pyridine (5 eq.) in dry DCM. The resulting mixture was stirred at room temperature for 1 hour and quenched with saturated aq. NaHCO₃ solution. The mixture was then extracted with EtOAc twice and the combined organic layers were washed with brine, dried over anhydrous Na₂SO₄, filtered and concentrated under vacuum. The residue was purified by column chromatography on silica gel (eluted with Petroleum Ether:EtOAc=10:1 to 2:1) to give compound 7.

### Step 5: Synthesis of Compound 9

The appropriate diamine 8 (2 eq.) was added under N₂ atmosphere to a mixture of compound 7 (1 eq.) in THF and EtOH (1:1, v/v), and the resulting mixture was stirred at 80° C. for 30 minutes. The mixture was then concentrated under vacuum to give compound 9.

### Step 6: Synthesis of Compound 10

10% Pd/C (20% w/w) was added to a solution of compound 9 (1 eq.) in THF, and the resulting mixture was stirred under 15 psi H₂ at room temperature for 30 minutes. The mixture was then filtered and the filtrate was concentrated under vacuum. The residue was purified by prep-HPLC (C18, 0% to 50% acetonitrile in H₂O with 0.1% NH₃.H₂O) to afford compound 10.

### Synthesis of A207

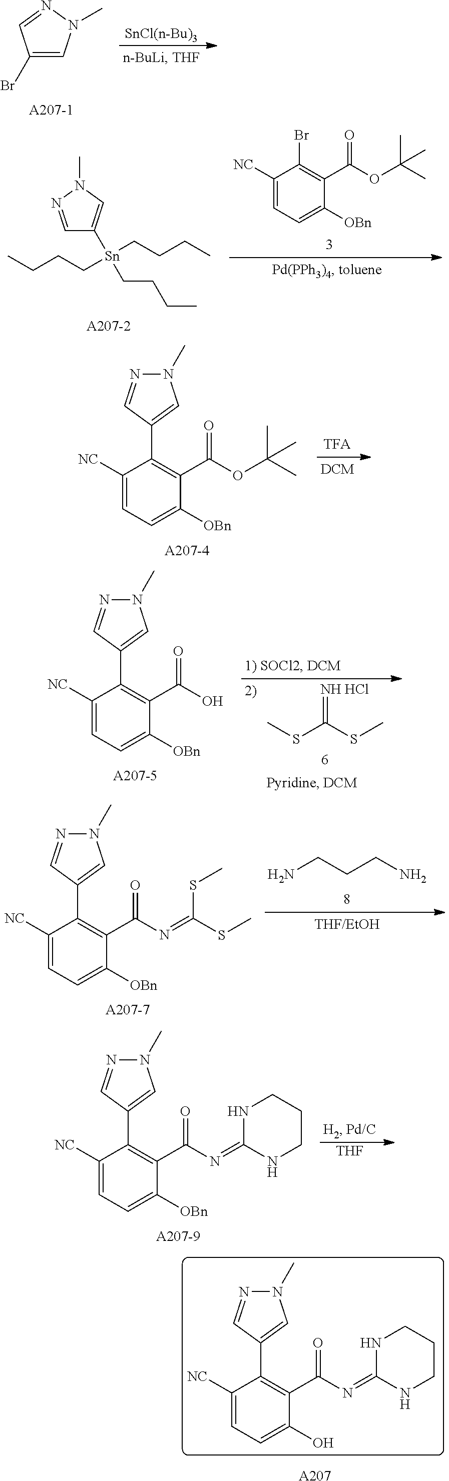

### Step 1: Synthesis of Compound A207-2

n-BuLi (8.2 mL, 8.2 mmol) was added dropwise at −20/−10° C. under N₂ atmosphere to a solution of compound A207-1 (1.2 g, 7.45 mmol) in THF (5 mL), and the resulting mixture was stirred at −10° C. for 20 minutes before a solution of Bu₃SnCl (3.15 g, 9.69 mmol) in anhydrous THF was added. The resulting mixture was stirred at room temperature for 16 hours. The mixture was then diluted with EtOAc and washed with saturated aq. NH₄Cl solution and brine, dried over Na₂SO₄, filtered and concentrated under vacuum. The residue was purified by column chromatography on silica gel (eluted with Petroleum Ether:EtOAc=100:0 to 25:1) to give compound A207-2 (1.02 g, 36.87% yield) as a white solid. LC/MS (ESI) m/z: 373(M+H)⁺.

### Step 2: Synthesis of Compound A207-4

Pd(PPh₃)₄ (95.8 mg, 0.008 mmol) was added under N₂ atmosphere to a suspension of 3 (322 mg, 0.83 mmol) and A207-2 (400 mg, 1.08 mmol) in toluene, and the resulting mixture was stirred at 100° C. for 20 hours. The mixture was then diluted with EtOAc and washed with water and brine, dried over Na₂SO₄, filtered and concentrated under vacuum. The residue was purified by column chromatography on silica gel (eluted with Petroleum Ether:EtOAc=10:1 to 5:1) to give compound A207-4 (107 mg, 33.14% yield) as a white solid. LC/MS (ESI) m/z: 390(M+H)⁺.

### Step 3: Synthesis of Compound A207-5

TFA (2 mL) was added under N₂ atmosphere to a mixture of compound A207-4 (107 mg, 0.27 mmol) in DCM (4 mL) and the resulting mixture was stirred at room temperature for 3 hours. The mixture was then concentrated under vacuum and the residue was purified by column chromatography on silica gel (eluted with Petroleum Ether:EtOAc=10:1 to 1:1) to give compound A207-5 (90 mg, 98.27% yield) as a white solid. LC/MS (ESI) m/z: 332(M−H)⁻.

### Step 4: Synthesis of Compound A207-7

Thionyl chloride (160.6 mg, 1.35 mmol) was added to a suspension of compound A207-5 (90 mg, 270.0 μmol) in anhydrous DCM, and the resulting mixture was stirred at 50° C. for 4-6 hours. After the reaction was completed, the mixture was concentrated under vacuum and the crude product was dissolved in anhydrous DCM and added to a mixture of 6 (63.86 mg, 405.0 μmol) and Pyridine (106.8 mg, 1.35 mmol) in dry DCM. The resulting mixture was stirred at room temperature for 1 hour and quenched with saturated aq. NaHCO₃ solution. The mixture was then extracted with EtOAc and the organic layer was separated, dried over anhydrous Na₂SO₄, filtered and concentrated under vacuum. The residue was purified by column chromatography on silica gel (eluted with Petroleum Ether:EtOAc=10:1 to 2:1) to give compound A207-7 (87 mg, 73.81% yield) as a white solid. LC/MS (ESI) m/z: 437(M+H)⁺.

### Step 5: Synthesis of Compound A207-9

Compound 8 (29.55 mg, 398.6 μmol) was added under N₂ atmosphere to a mixture of compound A207-7 (87 mg, 199.3 μmol) in THF (1.5 mL) and EtOH (1.5 mL), and the resulting mixture was stirred at 80° C. for 30 minutes. The mixture was then concentrated under vacuum to give compound A207-9 (81 mg, 98.06% yield) as a white solid. LC/MS (ESI) m/z: 415(M+H)⁺.

### Step 6: Synthesis of Compound A207

10% Pd/C (16.2 mg) was added to a solution of compound A207-9 (81 mg, 195.4 μmol) in THF (3 mL), and the resulting mixture was stirred under H₂ atmosphere at room temperature for 30 minutes. The mixture was then filtered and the filtrate was concentrated under vacuum. The residue was purified via prep-HPLC (C18, 0% to 50% acetonitrile in H₂O with 0.1% NH₃.H₂O) to afford compound A207 (50 mg, 78.88% yield) as a white solid. LC/MS (ESI) m/z: 325(M+H)⁺.

The compounds in the table below were prepared from the appropriate starting materials described previously or commercially available using the above general procedure 12.

| Structure | Name |
|---|---|
| 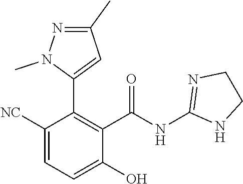 [SMILES: Cc1cc(-c2c(C#N)ccc(O)c2C(=O)NC2=NCCN2)n(C)n1] | 3-cyano-N-(4,5-dihydro-1H-imidazol-2-yl)-2- (1,3-dimethyl-1H-pyrazol-5-yl)-6- hydroxybenzamide LC-MS: m/z 325 (M + H)⁺. 1H NMR (400 MHz, DMSO) δ 8.63 (s, 2H), 7.71 (d, J = 8.7 Hz, 1H), 7.00 (d, J = 8.7 Hz, 1H), 5.92 (s, 1H), 3.55 (s, 4H), 3.40 (s, 3H), 2.15 (s, 3H). |
| 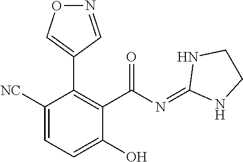 [SMILES: N#Cc1ccc(O)c(C(=O)N=C2NCCN2)c1-c1cnoc1] | 3-cyano-6-hydroxy-N-(imidazolidin-2-ylidene)-2- (isoxazol-4-yl)benzamide LC-MS: m/z 383 (M + H)⁺. 1H NMR (400 MHz, DMSO) δ 8.98 (s, 1H), 8.63 (br s, 2H), 8.60 (s, 1H), 7.71 (d, J = 8.7 Hz, 1H), 6.97 (d, J = 8.7 Hz, 1H), 3.56 (s, 4H). |
| 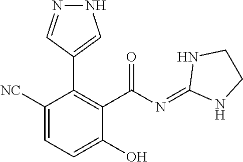 [SMILES: N#Cc1ccc(O)c(C(=O)N=C2NCCN2)c1-c1cn[nH]c1] | 3-cyano-6-hydroxy-N-(imidazolidin-2-ylidene)-2- (1H-pyrazol-4-yl)benzamide LC-MS: m/z 297 (M + H)⁺. 1H NMR (400 MHz, DMSO) δ 15.46 (s, 1H), 12.79 (s, 1H), 8.46 (s, 2H), 7.73 (s, 1H), 7.61 (d, J = 8.6 Hz, 1H), 7.44 (s, 1H), 6.86 (d, J = 8.6 Hz, 1H), 3.53 (s, 4H). |
| 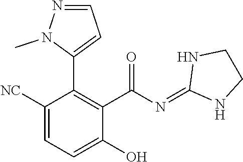 [SMILES: Cn1nccc1-c1c(C#N)ccc(O)c1C(=O)N=C1NCCN1] | 3-cyano-6-hydroxy-N-(imidazolidin-2-ylidene)-2- (1-methyl-1H-pyrazol-5-yl)benzamide LC-MS: m/z 311 (M + H)⁺. 1H NMR (400 MHz, DMSO) δ 8.63 (s, 2H), 7.74 (d, J = 8.7 Hz, 1H), 7.39 (d, J = 1.8 Hz, 1H), 7.02 (d, J = 8.7 Hz, 1H), 6.15 (d, J = 1.8 Hz, 1H), 3.56 (s, 4H), 3.50 (s, 3H). |
| 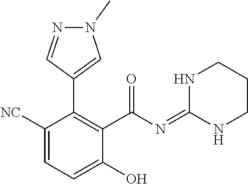 [SMILES: Cn1cc(-c2c(C#N)ccc(O)c2C(=O)N=C2NCCCN2)cn1] | 3-cyano-6-hydroxy-2-(1-methyl-1H-pyrazol-4- yl)-N-(tetrahydropyrimidin-2(1H)- ylidene)benzamide LC-MS: m/z 325 (M + H)⁺. 1H NMR (400 MHz, DMSO) δ 7.61 (d, J = 8.8 Hz, 1H), 7.36 (d, J = 1.8 Hz, 1H), 6.88 (d, J = 8.8 Hz, 1H), 6.10 (d, J = 1.8 Hz, 1H), 3.49 (s, 3H), 3.28-3.24 (m, 4H), 1.82-1.74 (m, 2H). |

### General Procedure 13

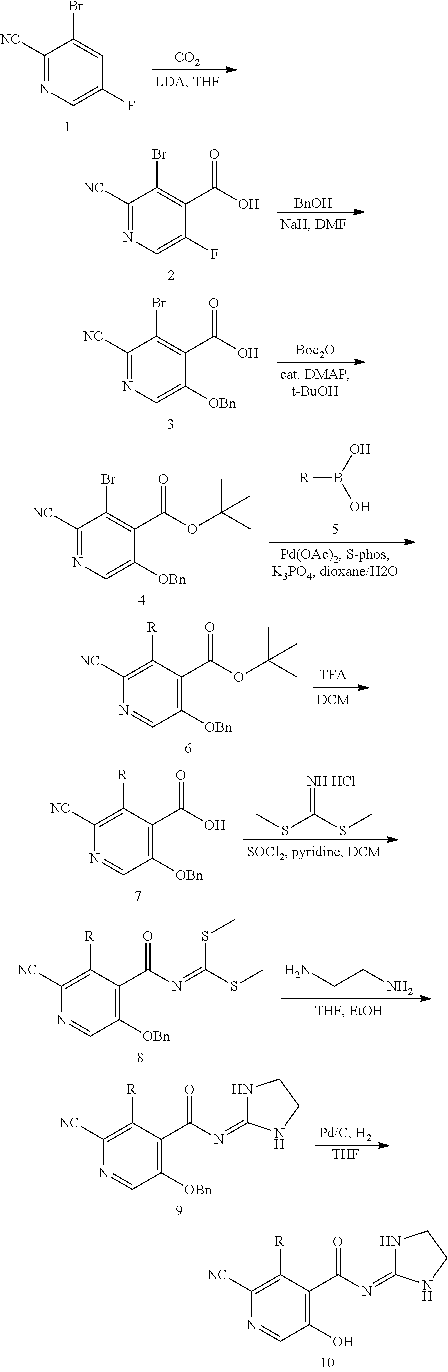

### Step 1: Synthesis of Compound 2

LDA (1.75 mL, 2M in THF) was added at −78° C. under N₂ atmosphere to a solution of compound 1 (500 mg, 2.5 mmol) in anhydrous THF (10 mL), and the resulting mixture was stirred at −78° C. for 1 hour. CO₂ (gas) was then bubbled into the above mixture at −78° C. for 30 minutes. When the reaction was completed a mixture of ice/water was added and the mixture was extracted with methyl tert-butyl ether twice. The aqueous layer was separated, acidified with 0.5 M aq. HCl solution and extracted with EtOAc twice. The combined organic layers were washed with brine, dried over Na₂SO₄, filtered and concentrated under vacuum to give compound 2 (320 mg, 52.46% yield) as a yellow solid. LC/MS (ESI) m/z: 243 (M−H)⁻.

### Step 2: Synthesis of Compound 3

60% NaH (110 mg, 2.75 mmol) was added at 0° C. under N₂ atmosphere to a solution of benzyl alcohol (0.2 mL, 1.967 mmol) in anhydrous DMF (10 mL), and the resulting mixture was stirred at room temperature for 1 hour before compound 2 (320 mg, 1.31 mmol) was added. The resulting mixture was then stirred at room temperature for 1.5 hours. The reaction mixture was then quenched with ice/H₂O and extracted with methyl tert-butyl ether twice. The aqueous layer was separated, acidified with 0.5 M aq.HCl solution and extracted with EtOAc twice. The combined organic layers were washed with brine, dried over anhydrous Na₂SO₄, filtered and concentrated to give compound 3 (420 mg, 96.46% yield) as a yellow solid without any further purification. LC/MS (ESI) m/z: 331 (M−H)⁻.

### Step 3: Synthesis of Compound 4

di-tert-butyl dicarbonate (0.6 mL, 2.53 mmol) and DMAP (23 mg, 0.19 mmol) were added to a solution of compound 3 (420 mg, 1.265 mmol) in t-BuOH (10 mL), and the resulting mixture was stirred at 60° C. for 16 hours. The mixture was then cooled to room temperature and concentrated under vacuum. The residue was purified by column chromatography on silica gel (Petroleum Ether:EtOAc=50:1 to 10:1) to give compound 4 (300 mg, 61.12% yield) as a white solid. LC/MS (ESI) m/z: 389 (M+H)⁺.

### Step 4: Synthesis of Compound 6

K₃PO₄ (2.5 eq) was added under N₂ atmosphere to a mixture of compound 4 (1.0 eq) and the appropriate phenylboronic acid 5 (1.5 eq) in dioxane and H₂O (8:1, V: V) followed by S-Phos (0.1 eq) and Pd(OAc)₂ (0.1 eq). The resulting reaction mixture was stirred at 95° C. for 16 hours under N₂ atmosphere. The mixture was then filtered and the filtrate was extracted with EtOAc twice. The combined organic layers were washed with brine, dried over anhydrous Na₂SO₄, filtered and concentrated under vacuum. The residue was purified by column chromatography on silica gel (eluted with Petroleum Ether:EtOAc=50:1 to 10:1) to give compound 6.

### Step 5: Synthesis of Compound 7

TFA (2:1, v/v) was added at 0° C. to a solution of compound 6 (1.0 eq) in DCM and the mixture was stirred at room temperature for 2 hours. The mixture was then concentrated to give compound 7, which was used for next step without any further purification.

### Step 6: Synthesis of Compound 8

SOCl₂ (10.0 eq) was added at 0° C. to a solution of compound 7 (1.0 eq) in DCM, and the resulting mixture was stirred at 60° C. for 2 hours. The mixture was then concentrated under vacuum and the residue was added to a mixture of dimethyl carbonimidodithioate (1.5 eq) and anhydrous pyridine (1.5 eq) in dry DCM at 0° C. The reaction was then stirred at room temperature for 30 min and concentrated under vacuum. The residue was purified by column chromatography on silica gel (Petroleum Ether:EtOAc=10:1 to 5:1) to give compound 8.

### Step 7: Synthesis of Compound 9

The appropriate diamine (2.0 eq) was added to a solution of compound 8 (1.0 eq) in THF and EtOH (1:1, v/v) and the mixture was stirred at 80° C. for 1 hour. The mixture was then concentrated under vacuum and the residue was purified by column chromatography on silica gel (eluted with DCM:MeOH=80:1 to 30:1) to give compound 9.

### Step 8: Synthesis of Compound 10

10% Pd/C (1:1, w/w) was added to a solution of compound 9 (1.0 eq) in THF, and the mixture was degassed under N₂ atmosphere for three times and stirred under H₂ atmosphere at room temperature for 30 minutes. The mixture was then filtered and the filtrate was concentrated under vacuum. The residue was purified via prep-HPLC (C18, 0% to 50% acetonitrile in H₂O with 0.1% NH₃.H₂O) to give compound 10.

### Synthesis of A392

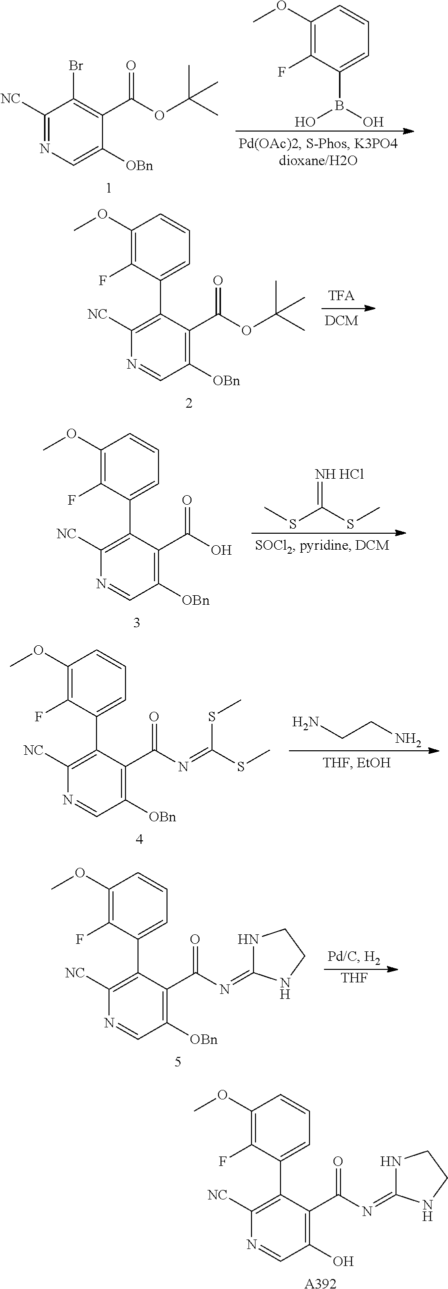

### Step 1: Synthesis of Compound A392-2

K₃PO₄ (369 mg, 1.74 mmol) was added under N₂ atmosphere to a mixture of compound A392-1 (270 mg, 0.70 mmol) and (2-fluoro-3-methoxyphenyl)boronic acid (177 mg, 1.04 mmol) in dioxane (8 mL) and H₂O (1 mL) followed by S-Phos (29 mg, 0.07 mmol) and Pd(OAc)₂ (16 mg, 0.07 mmol). The reaction mixture was stirred at 95° C. for 16 hours under N₂ atmosphere. The mixture was then filtered and the filtrate extracted with EtOAc twice. The combined organic layers were washed with brine, dried over anhydrous Na₂SO₄, filtered and concentrated under vacuum. The residue was purified by column chromatography on silica gel (eluted with Petroleum Ether:EtOAc=50:1 to 10:1) to give compound A392-2 as a yellow solid (170 mg, 56.29% yield). LC/MS (ESI) m/z: 435 (M+H)⁺.

### Step 2: Synthesis of Compound A392-3

TFA (1.5 mL) was added at 0° C. to a solution of compound A392-2 (170 mg, 0.39 mmol) in DCM (3 mL) and the mixture was stirred at room temperature for 2 hours. The mixture was then concentrated under vacuum to give compound A392-3 as a yellow solid (148 mg, 99.96% yield) without any further purification. LC/MS (ESI) m/z: 377 (M−H)⁻.

### Step 3: Synthesis of Compound A392-4

Thionyl chloride (466 mg, 3.82 mmol) was added at 0° C. to a solution of compound A392-3 (148 mg, 0.38 mmol) in DCM (4 ml) and the mixture was then stirred at 60° C. for 2 hours. The mixture was concentrated under vacuum and the crude product was added to a mixture of dimethyl carbonimidodithioate (86 mg, 0.55 mmol) and anhydrous pyridine (47 mg, 0.55 mmol) in dry DCM (3 mL) at 0° C. The resulting mixture was then stirred at room temperature for 30 minutes and concentrated under vacuum. The residue was purified by column chromatography on silica gel (Petroleum Ether:EtOAc=10:1 to 5:1) to give compound A392-4 as a light yellow solid (90 mg, 47.79% yield). LC/MS (ESI) m/z: 482 (M+H)⁺.

### Step 4: Synthesis of Compound A392-5

Ethylenediamine (23 mg, 0.38 mmol) was added to a solution of compound A392-4 (90 mg, 0.19 mmol) in THF (2 mL) and EtOH (2 mL), and the reaction was stirred at 80° C. for 1 hour. The mixture was then concentrated under vacuum and the residue was purified by column chromatography on silica gel (DCM:MeOH=80:1 to 30:1) to give compound A392-5 as a light yellow solid (70 mg, 84.07% yield). LC/MS (ESI) m/z: 446 (M+H)⁺.

### Step 5: Synthesis of Compound A392

10% Pd/C (70 mg) was added to a solution of compound A392-5 (70 mg, 0.16 mmol) in THF (3 mL), and the mixture was degassed under N₂ atmosphere for three times and stirred under H₂ atmosphere at room temperature for 30 minutes. The mixture was then filtered and the filtrate was concentrated under vacuum. The residue was purified via prep-HPLC (C18, 0% to 50% acetonitrile in H₂O with 0.1% NH₃.H₂O) to give compound A392 as a white solid (9.7 mg, 17.37% yield). LC/MS (ESI) m/z: 356 (M+H)⁺. ¹H NMR (400 MHz, DMSO) δ 9.01 (s, 2H), 8.34 (d, J=6.8 Hz, 1H), 7.24-7.18 (m, 2H), 6.93-6.85 (m, 1H), 3.92 (s, 3H), 3.64 (s, 4H).

The compounds in the table below were prepared from the appropriate starting materials described previously or commercially available using the above general procedure 13.

| Structure | Name |
|---|---|
| 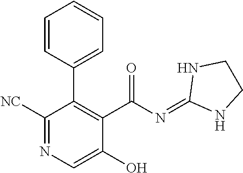 [SMILES: N#Cc1ncc(O)c(C(=O)N=C2NCCN2)c1-c1ccccc1] | 2-cyano-5-hydroxy-N-(imidazolidin-2-ylidene)-3- phenylisonicotinamide LC-MS: m/z 308 (M + H)⁺. 1H NMR (400 MHz, DMSO) δ 8.87 (s, 2H), 8.24 (s, 1H), 7.42-7.33 (m, 3H), 7.29-7.22 (m, 2H), 3.56 (s, 4H). |
| 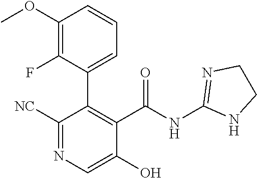 [SMILES: COc1cccc(-c2c(C#N)ncc(O)c2C(=O)NC2=NCCN2)c1F] | 2-cyano-N-(4,5-dihydro-1H-imidazol-2-yl)-3-(2- fluoro-3-methoxyphenyl)-5- hydroxyisonicotinamide LC-MS: m/z 356 (M + H)⁺. 1H NMR (400 MHz, DMSO) δ 9.01 (br s, 2H), 8.34 (d, J = 6.8 Hz, 1H), 7.24-7.18 (m, 2H), 6.93- 6.85 (m, 1H), 3.92 (s, 3H), 3.64 (s, 4H). |
| 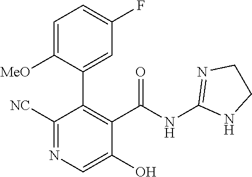 [SMILES: COc1ccc(F)cc1-c1c(C#N)ncc(O)c1C(=O)NC1=NCCN1] | 2-cyano-N-(4,5-dihydro-1H-imidazol-2-yl)-3-(5- fluoro-2-methoxyphenyl)-5- hydroxyisonicotinamide LC-MS: m/z 356 (M + H)⁺. 1H NMR (400 MHz, DMSO-d6) δ 8.88 (s, 2H), 8.23 (s, 1H), 7.18-7.13 (m, 1H), 7.06-7.02 (m, 2H), 3.66 (s, 3H), 3.57 (s, 4H). |
| 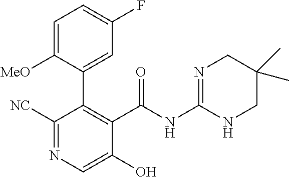 [SMILES: COc1ccc(F)cc1-c1c(C#N)ncc(O)c1C(=O)NC1=NCC(C)(C)CN1] | 2-cyano-N-(5,5-dimethyl-1,4,5,6- tetrahydropyrimidin-2-yl)-3-(5-fluoro-2- methoxyphenyl)-5-hydroxyisonicotinamide LC-MS: m/z 398 (M + H)⁺. 1H NMR (400 MHz, DMSO-d6) δ 9.16 (s, 2H), 8.15 (s, 1H), 7.18-7.12 (m, 1H), 7.07-6.96 (m, 2H), 3.66 (s, 3H), 2.98 (s, 4H), 0.94 (s, 6H). |
| 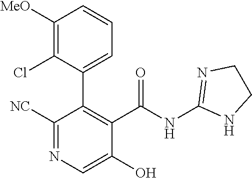 [SMILES: COc1cccc(-c2c(C#N)ncc(O)c2C(=O)NC2=NCCN2)c1Cl] | 3-(2-chloro-3-methoxyphenyl)-2-cyano-N-(4,5- dihydro-1H-imidazol-2-yl)-5- hydroxyisonicotinamide LC-MS: m/z 372 (M + H)⁺. 1H NMR (400 MHz, DMSO) δ 8.88 (s, 1H), 8.22 (s, 1H), 7.31 (dd, J = 8.0, 8.0 Hz, 1H), 7.12 (dd, J = 8.0, 1.2 Hz, 1H), 6.85 (dd, J = 8.0, 1.3 Hz, 1H), 3.88 (s, 3H), 3.54 (s, 4H) |

### General Procedure 14

### Method A:

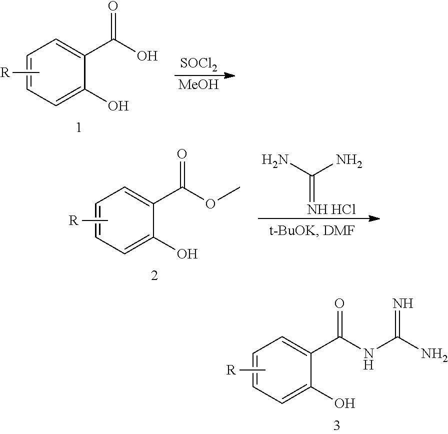

### Step 1: Synthesis of Compound 2

Thionyl chloride (10 eq.) was added at 0° C. to a solution of compound 1 (1 eq.) in MeOH and the resulting mixture was stirred at 80° C. under N₂ atmosphere for 16 hours. Then the mixture was concentrated under vacuum and the residue was purified by column chromatography on silica gel (eluted with Petroleum Ether:EtOAc=100:0 to 20:1) to give compound 2.

### Step 2: Synthesis of Compound 3

t-BuOK (8 eq.) was added under N₂ atmosphere to a suspension of guanidine hydrochloride (10 eq.) in dry DMF. The mixture was stirred at room temperature for 45 minutes before a solution of compound 2 (1 eq.) in DMF was added. The resulting mixture was stirred at room temperature for 16 hours. The mixture was diluted with EtOAc and washed with saturated aq. NH₄Cl solution and brine, dried over Na₂SO₄, filtered and concentrated under vacuum. The residue was purified via prep-HPLC (C18, 0% to 50% acetonitrile in H₂O with 0.1% NH₃.H₂O) to afford compound 3.

### Synthesis of A22

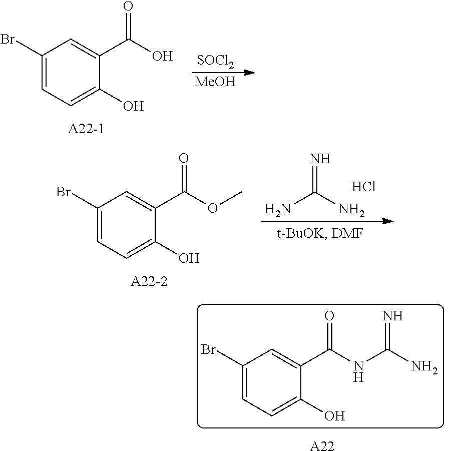

### Step 1: Synthesis of Compound A22-2

Thionyl chloride (2.38 g, 20 mmol) was added dropwise at 0° C. to a solution of compound A22-1 (432 mg, 2 mmol) in MeOH (20 mL), and the resulting mixture was stirred at 80° C. for 16 hours. The mixture was then concentrated under vacuum and the residue was purified by column chromatography on silica gel (eluted with Petroleum Ether:EtOAc=100:0 to 15:1) to give compound A22-2 (313 mg, 68% yield) as a colorless oil. LC/MS (ESI) m/z: 231 (M+H)⁺.

### Step 2: Synthesis of Compound A22

t-BuOK (1.22 g, 10.88 mmol) was added under N₂ atmosphere to a suspension of guanidine hydrochloride (1.30 g, 13.6 mmol) in dry DMF (25 mL). The resulting mixture was stirred at room temperature for 45 minutes before a solution of compound 2 (313 mg, 1.36 mmol) in DMF (2 mL) was added. The resulting mixture was stirred at room temperature for 16 hours. The mixture was then diluted with EtOAc and washed with saturated aq. NH₄Cl solution and brine, dried over Na₂SO₄, filtered and concentrated under vacuum. The residue was purified via prep-HPLC (C18, 0% to 50% acetonitrile in H₂O with 0.1% NH₃.H₂O) to give compound A22 (69 mg, 20% yield) as a white solid. LC/MS (ESI) m/z: 258 (M+H)⁺. 1H NMR (400 MHz, DMSO-d6) δ 14.94 (s, 1H), 8.37 (br s, 2H), 7.89 (d, J=2.7 Hz, 1H), 7.42 (dd, J=8.7, 2.7 Hz, 1H), 7.10 (br s, 1H), 6.76 (d, J=8.7 Hz, 1H).

### Method B:

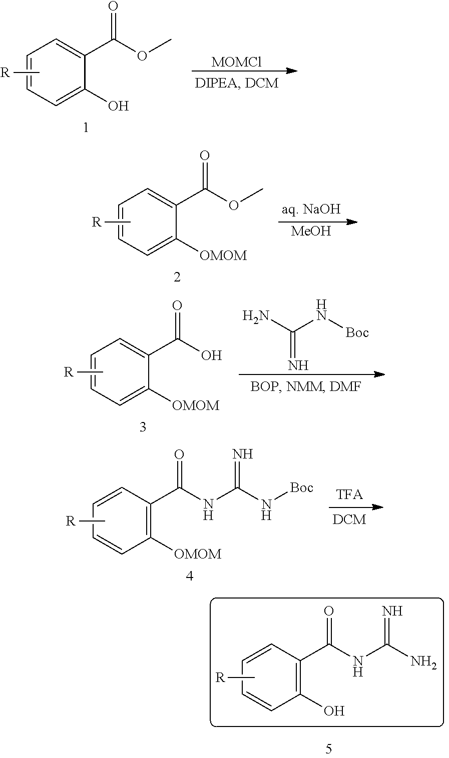

### Step 1: Synthesis of Compound 2

MOMCl (1.6 eq.) was added dropwise at 0° C. to a mixture of compound 1 (1 eq) and DIPEA (2.5 eq.) in dry DCM, and the resulting mixture was stirred at room temperature for 16 hours. The mixture was then quenched with saturated aq. NaHCO₃ solution and extracted with EtOAc twice. The combined organic layers were washed with brine, dried over anhydrous Na₂SO₄, filtered and concentrated under vacuum. The residue was purified by column chromatography on silica gel (eluted with Petroleum Ether:EtOAc=100:0 to 30:1) to give compound 2.

### Step 2: Synthesis of Compound 3

A solution of NaOH (3 eq.) in water was added to a mixture of compound 2 (1 eq.) in MeOH, and the resulting mixture was stirred at room temperature for 16 hours. The mixture was then diluted with water and extracted with diethylether. The aqueous layer was separated and acidified with 1 N aq. HCl solution to pH=5. The mixture was extracted with EtOAc twice and the combined organic layers were washed with brine, dried over anhydrous Na₂SO₄, filtered and concentrated under vacuum. The residue was purified by column chromatography on silica gel (eluted with Petroleum Ether:EtOAc=100:0 to 1:1) to give compound 3.

### Step 3: Synthesis of Compound 4

NMM (4 eq.) and PyBOP (1.5 eq.) were added under N₂ atmosphere to a mixture of compound 3 (1 eq.) and tert-butoxycarbonylguanidine (2 eq.) in dry DMF. The resulting mixture was stirred at room temperature for 16 hours. The mixture was then diluted with saturated aq. NH₄Cl solution and extracted with EtOAc twice. The combined organic layers were dried over anhydrous Na₂SO₄, filtered and concentrated under vacuum. The residue was purified by column chromatography on silica gel (eluted with Petroleum Ether:EtOAc=100:0 to 5:1) to give compound 4.

### Step 4: Synthesis of Compound 5

TFA (1:1, v/v) was added at 0° C. under N₂ atmosphere to a solution of compound 4 (1 eq.) in DCM, and the mixture was stirred at room temperature for 3 hours. The mixture was then concentrated under vacuum and the residue was purified via prep-HPLC (C18, 0% to 50% acetonitrile in H₂O with 0.1% NH₃.H₂O) to give compound 5.

### Synthesis of A43

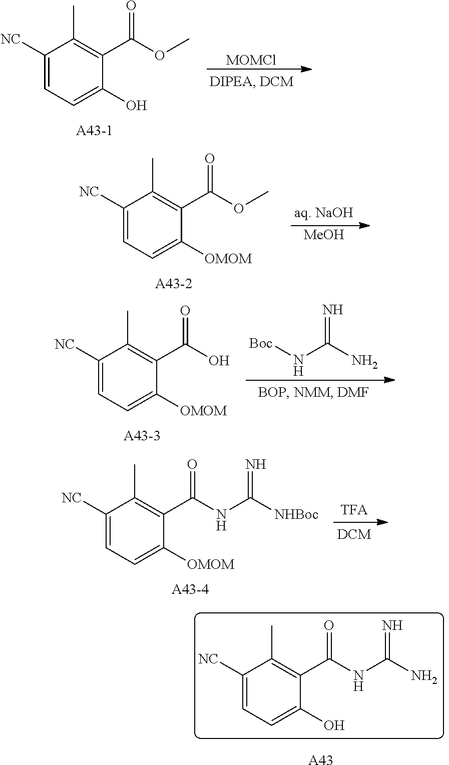

### Step 1: Synthesis of Compound A43-2

MOMCl (258 mg, 3.2 mmol) was added dropwise at 0° C. to a mixture of compound A43-1 (382 mg, 2 mmol) and DIPEA (650 mg, 5 mmol) in dry DCM (20 mL), and the resulting mixture was stirred at room temperature under N₂ atmosphere for 16 hours. The mixture was then quenched with saturated aq. NaHCO₃ solution and extracted with EtOAc twice. The organic layers were combined, dried over anhydrous Na₂SO₄, filtered and concentrated under vacuum. The residue was purified by column chromatography on silica gel (eluted with Petroleum Ether:EtOAc=100:0 to 30:1) to give compound A43-2 (310 mg, 66% yield) as a colorless oil. LC/MS (ESI) m/z: 258 (M+Na)⁺.

### Step 2: Synthesis of Compound A43-3

A solution of NaOH (158 mg, 3.96 mmol) in water (4 mL) was added to a solution of compound A43-2 (310 mg, 1.32 mmol) in MeOH (8 mL), and the resulting mixture was stirred at room temperature for 16 hours. The mixture was then diluted with water and extracted with ether. The aqueous layer was separated and acidified with 1 N aq. HCl solution to pH=5. The mixture was extracted with EtOAc twice and the combined organic layers were dried over anhydrous Na₂SO₄, filtered and concentrated under vacuum. The residue was purified by column chromatography on silica gel (eluted with Petroleum Ether:EtOAc=30:1 to 2:1) to give compound A43-3 (180 mg, 62% yield) as a colorless oil. LC/MS (ESI) m/z: 220 (M−H)⁻.

### Step 3: Synthesis of Compound A43-4

NMM (328 mg, 3.24 mmol) and PyBOP (537 mg, 1.22 mmol) were added under N₂ atmosphere to a mixture of compound A43-3 (180 mg, 0.81 mmol) and tert-butoxycarbonylguanidine (258 mg, 1.62 mmol) in dry DMF (8 mL). The resulting mixture was stirred at room temperature for 16 hours. The mixture was then diluted with saturated aq. NH₄Cl solution and extracted with EtOAc twice. The organic layers were combined, dried over anhydrous Na₂SO₄, filtered and concentrated under vacuum. The residue was purified by column chromatography on silica gel (eluted with Petroleum Ether:EtOAc=40:1 to 4:1) to give compound A43-4 (170 mg, 58% yield) as a colorless oil. LC/MS (ESI) m/z: 363 (M+H)⁺.

### Step 4: Synthesis of Compound A43

TFA (1 mL) was added at 0° C. under N₂ atmosphere to a solution of compound 6 (170 mg, 0.47 mmol) in DCM (2 mL). The resulting mixture was stirred at room temperature for 3 hours, and then the mixture was concentrated under vacuum. The residue was purified via prep-HPLC (C18, 0% to 50% acetonitrile in H₂O with 0.1% NH₃.H₂O) to give compound A43 (35 mg, 34% yield) as a white solid. LC/MS (ESI) m/z: 219(M+H)⁺. 1H NMR (400 MHz, DMSO) δ 8.43 (br s, 2H), 7.51 (d, J=8.8 Hz, 1H), 7.30 (br s, 1H), 6.69 (d, J=8.8 Hz, 1H), 2.73 (s, 3H).

The compounds in the table below were prepared from the appropriate starting materials described previously or commercially available using the above general procedure 14 (method A or B).

| Structure | Name |
|---|---|
| 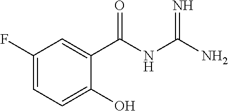 [SMILES: N=C(N)NC(=O)c1cc(F)ccc1O] | N-carbamimidoyl-5-fluoro-2- hydroxybenzamide LC-MS: m/z 198 (M + H)⁺. 1H NMR (400 MHz, DMSO) δ 14.47 (s, 1H), 8.35 (br s, 2H), 7.51-7.47 (m, 1H), 7.17-7.12 (m, 1H), 7.06 (br s, 1H), 6.80- 6.77 (m, 1H). |
| 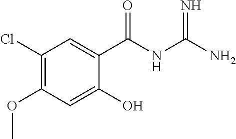 [SMILES: COc1cc(O)c(C(=O)NC(=N)N)cc1Cl] | N-carbamimidoyl-5-chloro-2-hydroxy-4- methoxybenzamide LC-MS: m/z 244 (M + H)⁺. 1H NMR (400 MHz, DMSO) δ 15.13 (s, 1H), 12.74 (s, 1H), 8.33 (br s, 2H), 7.72 (s, 1H), 7.04 (br s, 1H), 6.49 (s, 1H), 3.84 (s, 3H). |
| 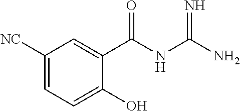 [SMILES: N#Cc1ccc(O)c(C(=O)NC(=N)N)c1] | N-carbamimidoyl-5-cyano-2- hydroxybenzamide LC-MS: m/z 205 (M + H)⁺. 1H NMR (400 MHz, DMSO) δ 8.49 (br s, 2H), 8.10 (s, 1H), 7.64 (d, J = 8.8 Hz, 1H), 7.34 (br s, 1H), 6.85 (d, J = 8.8 Hz, 1H). |
| 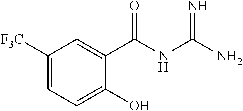 [SMILES: N=C(N)NC(=O)c1cc(C(F)(F)F)ccc1O] | N-carbamimidoyl-2-hydroxy-5- (trifluoromethyl)benzamide LC-MS: m/z 248 (M + H)⁺. 1H NMR (400 MHz, DMSO) δ 15.84 (s, 1H), 8.42 (br s, 2H), 8.09 (d, J = 4.0 Hz, 1H), 7.59 (d, J = 8.0 Hz, 1H), 7.24 (br s, 1H), 6.93 (d, J = 8.0 Hz, 1H). |
| 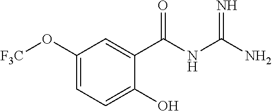 [SMILES: N=C(N)NC(=O)c1cc(OC(F)(F)F)ccc1O] | N-carbamimidoyl-2-hydroxy-5- (trifluoromethoxy)benzamide LC-MS: m/z 264 (M + H)⁺. 1H NMR (400 MHz, DMSO) δ 14.99 (s, 1H), 8.38 (br s, 2H), 7.69 (s, 1H), 7.29 (d, J = 8.0 Hz, 1H), 7.13 (br s, 1H), 6.87 (d, J = 8.0 Hz, 1H). |
| 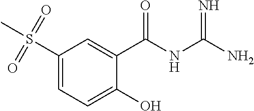 [SMILES: CS(=O)(=O)c1ccc(O)c(C(=O)NC(=N)N)c1] | N-carbamimidoyl-2-hydroxy-5- (methylsulfonyl)benzamide LC-MS: m/z 258 (M + H)⁺. 1H NMR (400 MHz, DMSO) δ 8.50 (br s, 2H), 8.30 (d, J = 2.8 Hz, 1H), 7.76 (dd, J = 8.8 Hz, 2.8 Hz, 1H), 7.29 (br s, 1H), 6.91 (d, J = 8.8 Hz, 1H), 3.12 (s, 3H). |
| 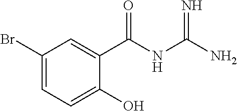 [SMILES: N=C(N)NC(=O)c1cc(Br)ccc1O] | 5-bromo-N-carbamimidoyl-2- hydroxybenzamide LC-MS: m/z 258 (M + H)⁺. 1H NMR (400 MHz, DMSO) δ 14.94 (s, 1H), 8.37 (br s, 2H), 7.89 (d, J = 2.7 Hz, 1H), 7.42 (dd, J = 8.7, 2.7 Hz, 1H), 7.10 (br s, 1H), 6.76 (d, J = 8.7 Hz, 1H). |
| 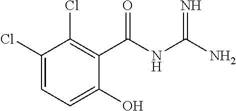 [SMILES: N=C(N)NC(=O)c1c(O)ccc(Cl)c1Cl] | N-carbamimidoyl-2,3-dichloro-6- hydroxybenzamide LC-MS: m/z 248 (M + H)⁺. 1H NMR (400 MHz, DMSO) δ 14.93 (br s, 1H), 8.20 (br s, 2H), 7.42 (d, J = 8.9 Hz, 1H), 7.14 (br s, 1H), 6.79 (d, J = 8.9 Hz, 1H). |
| 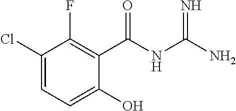 [SMILES: N=C(N)NC(=O)c1c(O)ccc(Cl)c1F] | N-carbamimidoyl-3-chloro-2-fluoro-6- hydroxybenzamide LC-MS: m/z 232 (M + H)⁺. 1H NMR (400 MHz, DMSO) δ 16.04 (s, 1H), 7.76 (br s, 3H), 7.38 (t, 1H), 6.63 (dd, J = 9.0, 1.5 Hz, 1H). |
| 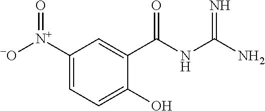 | N-carbamimidoyl-2-hydroxy-5- nitrobenzamide LC-MS: m/z 225 (M + H)⁺. 1H NMR (400 MHz, DMSO) δ 8.70 (br s, 2H), 8.65 (s, 1H), 8.06 (d, J = 7.1 Hz, 1H), 7.69 (br s, 1H), 6.72 (d, J = 9.2 Hz, 1H). |
| 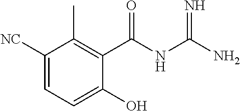 [SMILES: Cc1c(C#N)ccc(O)c1C(=O)NC(=N)N] | N-carbamimidoyl-3-cyano-6-hydroxy-2- methylbenzamide LC-MS: m/z 219 (M + H)⁺. 1H NMR (400 MHz, DMSO) δ 8.43 (br s, 2H), 7.51 (d, J = 8.8 Hz, 1H), 7.30 (br s, 1H), 6.69 (d, J = 8.8 Hz, 1H), 2.73 (s, 3H). |
| 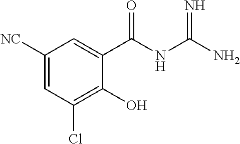 [SMILES: N#Cc1cc(Cl)c(O)c(C(=O)NC(=N)N)c1] | N-carbamimidoyl-3-chloro-5-cyano-2- hydroxybenzamide LC-MS: m/z 239 (M + H)⁺. 1H NMR (400 MHz, DMSO) δ 8.81 (br s, 2H), 7.97 (d, J = 2.3 Hz, 1H), 7.89 (br s, 1H), 7.77 (d, J = 2.3 Hz, 1H). |
| 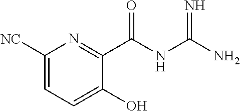 [SMILES: N#Cc1ccc(O)c(C(=O)NC(=N)N)n1] | N-carbamimidoyl-6-cyano-3- hydroxypicolinamide LC-MS: m/z 206 (M + H)⁺. 1H NMR (400 MHz, DMSO) δ 8.16 (br s, 3H), 7.73 (d, J = 8.1 Hz, 1H), 7.16 (d, J = 8.1 Hz, 1H). |
| 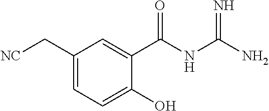 [SMILES: N#CCc1ccc(O)c(C(=O)NC(=N)N)c1] | N-carbamimidoyl-5-(cyanomethyl)-2- hydroxybenzamide LC-MS: m/z 219 (M + H)⁺. 1H NMR (400 MHz, DMSO) δ 14.82 (s, 1H), 8.36 (br s, 2H), 7.81 (d, J = 2.4 Hz, 1H), 7.24 (dd, J = 8.4, 2.5 Hz, 1H), 7.06 (br s, 1H), 6.79 (d, J = 8.4 Hz, 1H), 3.92 (s, 2H). |
| 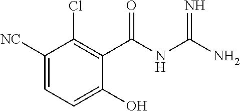 [SMILES: N#Cc1ccc(O)c(C(=O)NC(=N)N)c1Cl] | N-carbamimidoyl-2-chloro-3-cyano-6- hydroxybenzamide LC-MS: m/z 239 (M + H)⁺. 1H NMR (400 MHz, DMSO) δ 13.21 (br s, 1H), 8.58-8.28 (m, 4H), 7.85 (d, J = 8.8 Hz, 1H), 7.11 (d, J = 8.8 Hz, 1H). |
| 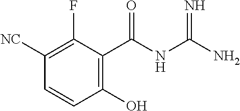 [SMILES: N#Cc1ccc(O)c(C(=O)NC(=N)N)c1F] | N-carbamimidoyl-3-cyano-2-fluoro-6- hydroxybenzamide LC-MS: m/z 223 (M + H)⁺. 1H NMR (400 MHz, DMSO) δ 8.44 (br s, 2H), 7.41 (t, J = 8.5 Hz, 1H), 6.47 (t, J = 11.6 Hz, 1H). |
| 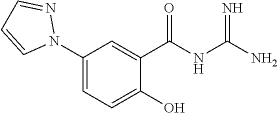 [SMILES: N=C(N)NC(=O)c1cc(-n2cccn2)ccc1O] | N-carbamimidoyl-2-hydroxy-5-(1H-pyrazol- 1-yl)benzamide LC-MS: m/z 246 (M + H)⁺. 1H NMR (400 MHz, DMSO) δ 14.86 (s, 1H), 8.39 (br s, 2H), 8.33 (d, J = 2.2 Hz, 1H), 8.19 (d, J = 2.8 Hz, 1H), 7.73 (dd, J = 8.8, 2.8 Hz, 1H), 7.67 (s, 1H), 7.08 (br s, 1H), |
| | 6.89 (d, J = 8.8 Hz, 1H), 6.47 (s, 1H). |
| 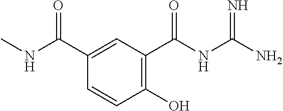 [SMILES: CNC(=O)c1ccc(O)c(C(=O)NC(=N)N)c1] | N3-carbamimidoyl-4-hydroxy-N1- methylisophthalamide LC-MS: m/z 237 (M + H)⁺. 1H NMR (400 MHz, DMSO) δ 15.52 (s, 1H), 8.37 (s, 1H), 8.26 (d, J =3.7 Hz, 1H), 7.83-7.48 (m, 4H), 6.79 (d, J =8.5 Hz, 1H), 2.75 (d, J =3.8 Hz, 3H). |
| 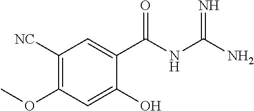 [SMILES: COc1cc(O)c(C(=O)NC(=N)N)cc1C#N] | N-carbamimidoyl-5-cyano-2-hydroxy-4- methoxybenzamide LC-MS: m/z 234 (M + H)⁺. 1H NMR (400 MHz, DMSO) δ 8.57 (br s, 2H), 7.96 (s, 1H), 7.38 (br s, 1H), 6.36 (s, 1H), 3.85 (s, 3H). |

### General Procedure 15

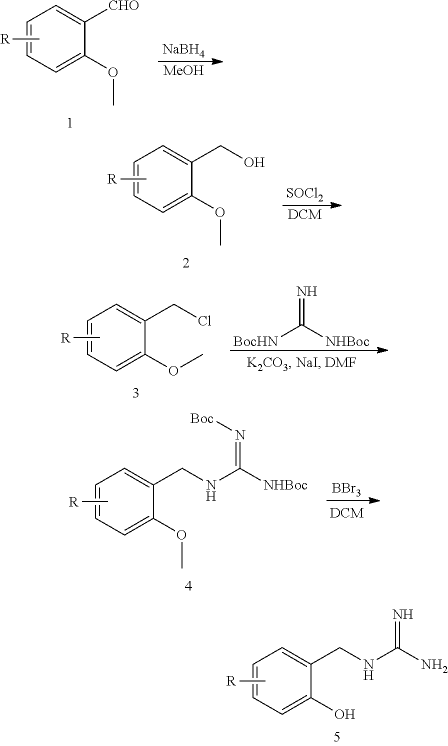

### Step 1: Synthesis of Compound 2

NaBH₄ (1.6 eq.) was added portion-wise at 0° C. to a solution of compound 1 (1 eq) in MeOH, and the resulting mixture was stirred at room temperature for 2 hours. The mixture was then quenched with saturated aq. NaHCO₃ solution and extracted with EtOAc twice. The combined organic layers were washed with brine, dried over anhydrous Na₂SO₄, filtered and concentrated under vacuum. The residue was purified by column chromatography on silica gel (eluted with Petroleum Ether:EtOAc=30:1 to 10:1) to give compound 2.

### Step 2: Synthesis of Compound 3

Thionyl chloride (3 eq.) was added dropwise at 0° C. to a solution of compound 2 (1 eq.) in dry DCM, and the resulting mixture was stirred at room temperature for 16 hours under N₂ atmosphere. The mixture was then concentrated under vacuum and the residue was purified by column chromatography on silica gel (eluted with Petroleum Ether:EtOAc=60:1 to 25:1) to give compound 3.

### Step 3: Synthesis of Compound 4

Potassium carbonate (2 eq.) and 1,3-Bis(tert-butoxycarbonyl)guanidine (1.5 eq.) were added to a mixture of compound 3 (1 eq.) and NaI (1 eq.) in DMF. The resulting mixture was stirred at 40° C. for 6 hours. The mixture was then cooled to room temperature, quenched with saturated aq. NH₄Cl solution and extracted with EtOAc twice. The combined organic layers were dried over anhydrous Na₂SO₄, filtered and concentrated under vacuum. The residue was purified by column chromatography on silica gel (eluted with Petroleum Ether:EtOAc=20:1 to 3:1) to give compound 4.

### Step 4: Synthesis of Compound 5

TFA (1:1, v/v) was added at 0° C. under N₂ atmosphere to a solution of compound 4 (1 eq.) in anhydrous DCM, and the resulting mixture was stirred at room temperature for 3 hours. The mixture was then concentrated under vacuum and the residue was purified via prep-HPLC (C18, 0% to 50% acetonitrile in H₂O with 0.1% NH₃.H₂O) to give compound 5.

### Synthesis of A2

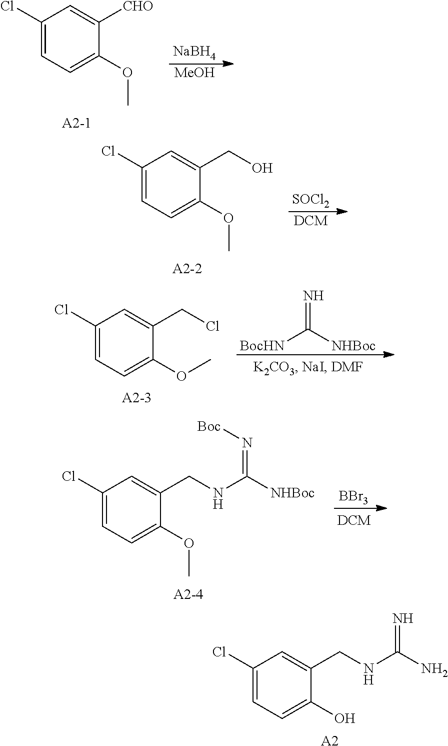

### Step 1: Synthesis of Compound A2-2

NaBH₄ (152 mg, 4 mmol) was added portion-wise at 0° C. to a solution of compound A2-1 (340 mg, 2 mmol) in MeOH (10 mL), and the resulting mixture was stirred at room temperature for 2 hours. The mixture was then quenched with saturated aq. NaHCO₃ solution and extracted with EtOAc twice. The combined organic layers were washed with brine, dried over anhydrous Na₂SO₄, filtered and concentrated under vacuum. The residue was purified by column chromatography on silica gel (eluted with Petroleum Ether:EtOAc=30:1 to 10:1) to give compound A2-2 (308 mg, 90% yield) as a colorless oil. LC/MS (ESI) m/z: 173(M+H)⁺.

### Step 2: Synthesis of Compound A2-3

Thionyl chloride (640 mg, 5.37 mmol) was added dropwise at 0° C. to a solution of compound A2-2 (308 mg, 1.79 mmol) in dry DCM (8 mL), and the resulting mixture was stirred at room temperature for 16 hours under N₂ atmosphere. The mixture was then concentrated under vacuum and the residue was purified by column chromatography on silica gel (eluted with Petroleum Ether:EtOAc=60:1 to 25:1) to give compound A2-3 (223 mg, 65% yield) as a light-yellow solid. LC/MS (ESI) m/z: 213(M+Na)⁺.

### Step 3: Synthesis of Compound A2-4

Potassium carbonate (323 mg, 2.34 mmol) and 1,3-Bis(tert-butoxycarbonyl)guanidine (454 mg, 1.76 mmol) were added to a mixture of compound A2-3 (223 mg, 1.17 mmol) and NaI (218 mg, 1.17 mmol) in DMF. The resulting mixture was stirred at 40° C. for 6 hours. The mixture was then cooled to room temperature, quenched with saturated aq. NH₄Cl solution and extracted with EtOAc twice. The combined organic layers were dried over anhydrous Na₂SO₄, filtered and concentrated under vacuum. The residue was purified by column chromatography on silica gel (eluted with Petroleum Ether:EtOAc=20:1 to 3:1) to give compound A2-4 (380 mg, 79% yield) as a white solid. LC/MS (ESI) m/z: 414(M+H)⁺.

### Step 4: Synthesis of Compound A2

TFA (2 mL) was added at 0° C. under N₂ atmosphere to a solution of compound A2-4 (380 mg, 0.92 mmol) in anhydrous DCM (2 mL), and the resulting mixture was stirred at room temperature for 3 hours. The mixture was then concentrated under vacuum and the residue was purified via prep-HPLC (C18, 0% to 50% acetonitrile in H₂O with 0.1% NH₃.H₂O) to give compound A2 (33 mg, 18% yield) as a white solid. LC/MS (ESI) m/z: 200(M+H)⁺. 1H NMR (400 MHz, MeOD) δ 8.53 (s, 2H), 7.21 (d, J=2.8 Hz, 1H), 7.16 (dd, J=8.8, 2.4 Hz, 1H), 6.82 (d, J=8.4 Hz, 1H), 4.32 (s, 2H).

The compounds in the table below were prepared from the appropriate starting materials described previously or commercially available using the above general procedure 15.

| Structure | Name |
|---|---|
| 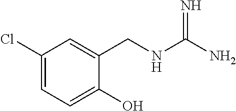 [SMILES: N=C(N)NCc1cc(Cl)ccc1O] | 1-(5-chloro-2-hydroxybenzyl)guanidine LC-MS: m/z 200 (M + H)⁺. 1H NMR (400 MHz, MeOD) δ 8.53 (s, 2H), 7.21 (d, J = 2.8 Hz, 1H), 7.16 (dd, J = 8.8, 2.4 Hz, 1H), 6.82 (d, J = 8.4 Hz, 1H), 4.32 (s, 2H). |
| 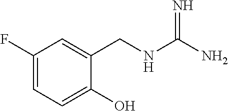 [SMILES: N=C(N)NCc1cc(F)ccc1O] | 1-(5-fluoro-2-hydroxybenzyl)guanidine LC-MS: m/z 184 (M + H)⁺. 1H NMR (400 MHz, MeOD) δ 7.32- 7.22 (m, 3H), 4.77 (s, 2H). |

### General Procedure 16

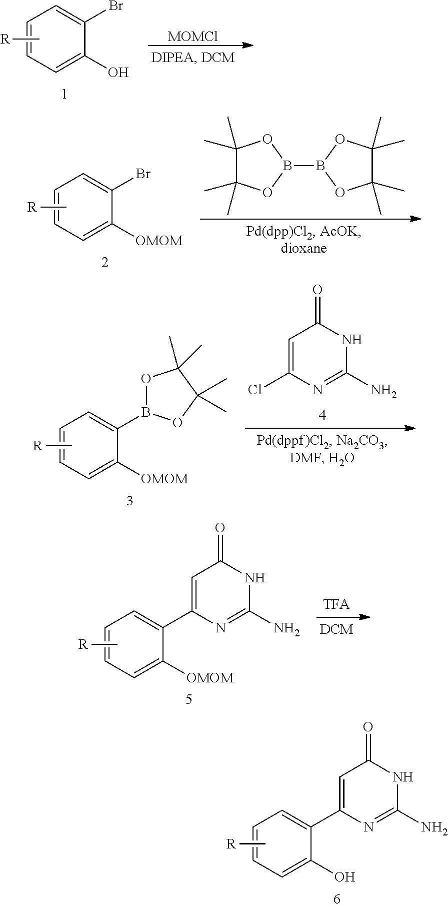

### Step 1: Synthesis of Compound 2

MOMCl (2 eq.) was added dropwise at 0° C. under N₂ atmosphere to a solution of compound 1 (1 eq) and DIPEA (3 eq.) in dry DCM. The resulting mixture was stirred at room temperature for 16 hours. The mixture was then quenched with saturated aq. NaHCO₃ solution and extracted with DCM twice. The combined organic layers were washed with brine, dried over anhydrous Na₂SO₄, filtered and concentrated under vacuum. The residue was purified by column chromatography on silica gel (eluted with Petroleum Ether:EtOAc=100:0 to 40:1) to give compound 2.

### Step 2: Synthesis of Compound 3

Potassium acetate (3 eq.) and Pd(dppf)Cl₂ (0.08 eq.) were added to a mixture of compound 2 (1 eq.) and bis(pinacolato)diboron (1.5 eq.) in 1,4-dioxane, and the resulting mixture was stirred at 100° C. for 16 hours under N₂ atmosphere. The mixture was then cooled to room temperature, diluted with EtOAc and washed with water. The organic layer was then dried over Na₂SO₄, filtered and concentrated under vacuum. The residue was purified by column chromatography on silica gel (eluted with Petroleum Ether:EtOAc=80:1 to 40:1) to give compound 3.

### Step 3: Synthesis of Compound 5

Sodium carbonate (3 eq.) and Pd(dppf)Cl₂ (0.08 eq.) were added to a mixture of compound 3 (1 eq.) and 2-amino-4-chloro-6-hydroxypyrimidine (1.5 eq.) in DMF and H₂O (10:1, v/v), and the resulting mixture was stirred at 90° C. for 16 hours under N₂ atmosphere. The mixture was then cooled to room temperature, diluted with EtOAc and washed with saturated aq. NH₄Cl solution. The organic layer was then dried over Na₂SO₄, filtered and concentrated under vacuum. The residue was purified by column chromatography on silica gel (eluted with Petroleum Ether:EtOAc=50:1 to 4:1) to give compound 5.

### Step 4: Synthesis of Compound 6

TFA (1:1, v/v) was added dropwise at 0° C. under N₂ atmosphere to a solution of compound 5 (1 eq.) in anhydrous DCM, and the resulting mixture was stirred at room temperature for 3 hours. The mixture was then concentrated under vacuum and the residue was purified via prep-HPLC (C18, 0% to 50% acetonitrile in H₂O with 0.1% NH₃.H₂O) to give compound 6.

### Synthesis of C8

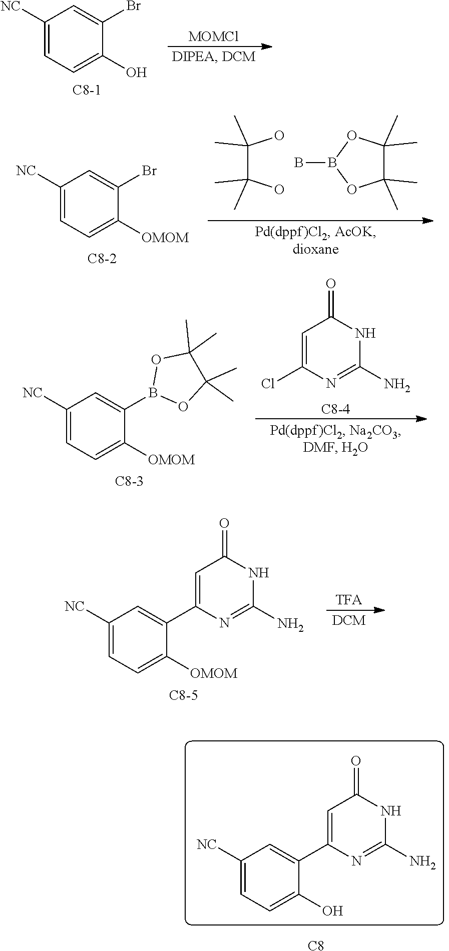

### Step 1: Synthesis of Compound C8-2

MOMCl (320 mg, 4 mmol) was added dropwise at 0° C. under N₂ atmosphere to a solution of compound C8-1 (396 mg, 2 mmol) and DIPEA (780 mg, 6 mmol) in dry DCM (10 mL). The resulting mixture was stirred at room temperature for 16 hours. The mixture was then quenched with saturated aq. NaHCO₃ solution and extracted with DCM twice. The combined organic layers were washed with brine, dried over anhydrous Na₂SO₄, filtered and concentrated under vacuum. The residue was purified by column chromatography on silica gel (eluted with Petroleum Ether:EtOAc=100:0 to 40:1) to give compound C8-2 (410 mg, 85% yield) as a colorless oil. LC/MS (ESI) m/z: 242(M+H)⁺.

### Step 2: Synthesis of Compound C8-3

Potassium acetate (500 mg, 5.1 mmol) and Pd(dppf)Cl₂ (99 mg, 0.136 mmol) were added to a mixture of compound C8-2 (410 mg, 1.70 mmol) and bis(pinacolato)diboron (650 mg, 2.55 mmol) in 1,4-dioxane, and the resulting mixture was stirred at 100° C. for 16 hours under N₂ atmosphere. The mixture was then cooled to room temperature, diluted with EtOAc and washed with water. The organic layer was then dried over Na₂SO₄, filtered and concentrated under vacuum. The residue was purified by column chromatography on silica gel (eluted with Petroleum Ether:EtOAc=80:1 to 40:1) to give compound C8-3 (270 mg, 55% yield) as a yellow solid. LC/MS (ESI) m/z: 290(M+H)⁺.

### Step 3: Synthesis of Compound C8-5

Sodium carbonate (296 mg, 2.79 mmol) and Pd(dppf)Cl₂ (54 mg, 0.074 mmol) were added to a mixture of compound C8-3 (270 mg, 0.93 mmol) and 2-amino-4-chloro-6-hydroxypyrimidine (203 mg, 1.4 mmol) in DMF and H₂O (11 mL, 10:1, v/v), and the resulting mixture was stirred at 90° C. for 16 hours under N₂ atmosphere. The mixture was then cooled to room temperature, diluted with EtOAc and washed with saturated aq. NH₄Cl solution. The organic layer was then dried over Na₂SO₄, filtered and concentrated under vacuum. The residue was purified by column chromatography on silica gel (eluted with Petroleum Ether:EtOAc=50:1 to 4:1) to give compound C8-5 (110 mg, 43% yield) as a yellow solid. LC/MS (ESI) m/z: 273(M+H)⁺.

### Step 4: Synthesis of Compound C8

TFA (2 mL) was added dropwise at 0° C. under N₂ atmosphere to a solution of compound C8-5 (110 mg, 0.4 mmol) in anhydrous DCM (2 mL), and the resulting mixture was stirred at room temperature for 3 hours. The mixture was then concentrated under vacuum and the residue was purified via prep-HPLC (C18, 0% to 50% acetonitrile in H₂O with 0.1% NH₃.H₂O) to give compound C8 (12 mg, 13% yield) as a white solid. LC/MS (ESI) m/z: 229(M+H)⁺. 1H NMR (400 MHz, DMSO-d6) δ 14.82 (br s, 1H), 11.23 (br s, 1H), 8.32 (d, J=2.0 Hz, 1H), 7.67 (dd, J=8.6, 2.0 Hz, 1H), 7.20 (s, 2H), 6.95 (d, J=8.6 Hz, 1H), 6.37 (s, 1H).

The compounds in the table below were prepared from the appropriate starting materials described previously or commercially available using the above general procedure 16.

| Structure | Name |
|---|---|
| 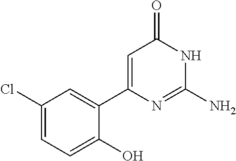 [SMILES: Nc1nc(-c2cc(Cl)ccc2O)cc(=O)[nH]1] | 2-amino-6-(5-chloro-2-hydroxyphenyl)- pyrimidin-4(3H)-one LC-MS: m/z 238 (M + H)⁺. 1H NMR (400 MHz, DMSO) δ 13.76 (s, 1H), 11.19 (s, 1H), 7.82 (d, J = 2.7 Hz, 1H), 7.30 (dd, J = 8.8, 2.6 Hz, 1H), 7.12 (br s, 2H), 6.84 (d, J = 8.8 Hz, 1H), 6.27 (s, 1H). |
| 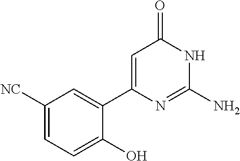 [SMILES: N#Cc1ccc(O)c(-c2cc(=O)[nH]c(N)n2)c1] | 3-(2-amino-6-oxo-1,6-dihydropyrimidin- 4-yl)-4-hydroxybenzonitrile LC-MS: m/z 229 (M + H)⁺. 1H NMR (400 MHz, DMSO) δ 14.82 (br s, 1H), 11.23 (br s, 1H), 8.32 (d, J = 2.0 Hz, 1H), 7.67 (dd, J = 8.6, 2.0 Hz, 1H), 7.20 (s, 2H), 6.95 (d, J = 8.6 Hz, 1H), 6.37 (s, 1H). |
| 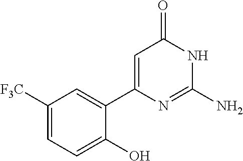 [SMILES: Nc1nc(-c2cc(C(F)(F)F)ccc2O)cc(=O)[nH]1] | 2-amino-6-(2-hydroxy-5-(trifluoro- methyl)phenyl)pyrimidin-4(3H)-one LC-MS: m/z 272 (M + H)⁺. 1H NMR (400 MHz, DMSO-d6) δ 14.29 (s, 1H), 11.17 (s, 1H), 8.08 (s, 1H), 7.59 (dd, J = 8.7, 2.0 Hz, 1H), 7.13 (br s, 2H), 7.00 (d, J = 8.6 Hz, 1H), 6.36 (s, 1H). |

### General Procedure 17

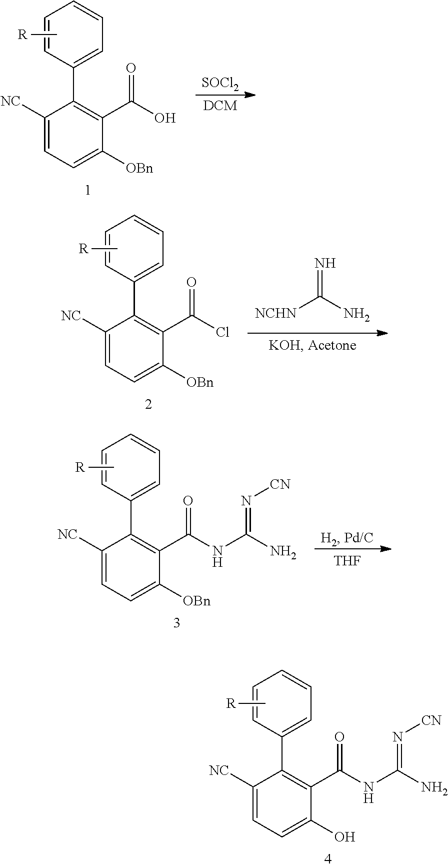

Intermediate 1 was prepared following general procedure 5

### Step 1: Synthesis of Compound 2

Thionyl chloride (10 eq) was added at 0° C. to a solution of compound 1 (1 eq) in DCM, and the mixture was stirred at 65° C. for 2 hours. The reaction mixture was then concentrated under vacuum to give compound 1 which was used in the next step without any further purification.

### Step 2: Synthesis of Compound 3

N-cyanoguanidine (1.25 eq) was added at 0° C. under N₂ atmosphere to a mixture of KOH (2 eq) in H₂O/Acetone, and the mixture was stirred at 10° C. for 1 hour. Then a solution of compound 2 (1 eq) in acetone was added dropwise to the above mixture and the resulting mixture was stirred at room temperature for 1 hour under N₂ atmosphere. The mixture was then poured into iced-water and extracted with EtOAc twice. The combined organic layers were washed with brine, dried over anhydrous Na₂SO₄, filtered and concentrated under vacuum. The residue was purified by column chromatography on silica gel (eluted with Petroleum Ether:EtOAc=15:1 to 4:1) to give compound 3.

### Step 3: Synthesis of Compound 4

10% Pd/C (1:1, w/w) was added at 0° C. to a solution of compound 3 (1 eq) in THF, and the mixture was degassed under N₂ atmosphere for three times and stirred under a H₂ at room temperature for 30 minutes. The mixture was then filtered and the filtrate was concentrated under vacuum. The residue was purified via prep-HPLC (C18, 0% to 50% acetonitrile in H₂O with 0.1% NH₃.H₂O) to give compound 4

### Synthesis of A129

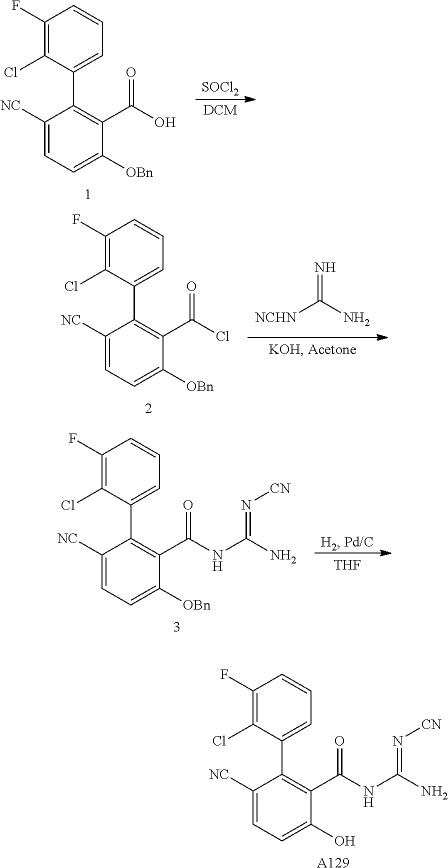

### Step 1: Synthesis of Compound A129-2

Thionyl chloride (0.2 mL, 3.0 mmol) was added at 0° C. to a solution of compound A129-1 (110 mg, 0.30 mmol) in DCM (4 mL), and the mixture was stirred at 65° C. for 2 hours. The reaction mixture was then concentrated under vacuum to give compound A129-2 (110 mg, 99% yield) as a light yellow solid which was used in the next step without any further purification.

### Step 2: Synthesis of Compound A129-3

N-cyanoguanidine (26 mg, 0.31 mmol) was added at 0° C. under N₂ atmosphere to a mixture of KOH (33 mg, 0.50 mmol) in H₂O (0.2 mL) and acetone (4 mL), and the mixture was stirred at 10° C. for 1 hour. Then a solution of compound A129-2 (100 mg, 0.25 mmol) in acetone (4 mL) was added dropwise to the above mixture and the resulting mixture was stirred at room temperature for 1 hour under N₂ atmosphere. The mixture was then poured into iced-water and extracted with EtOAc twice. The combined organic layers were washed with brine, dried over anhydrous Na₂SO₄, filtered and concentrated under vacuum. The residue was purified by column chromatography on silica gel (eluted with Petroleum Ether:EtOAc=15:1 to 4:1) to give compound A129-3 (62 mg, 55% yield) as a yellow solid. LC/MS (ESI) m/z: 448 (M+H)⁺.

### Step 3: Synthesis of Compound A129

10% Pd/C (60 mg) was added at 0° C. to a solution of compound A129-3 (60 mg, 0.13 mmol) in THF (4 mL), and the mixture was degassed under N₂ atmosphere for three times and stirred under a H₂ at room temperature for 30 minutes. The mixture was then filtered and the filtrate was concentrated under vacuum. The residue was purified via prep-HPLC (C18, 0% to 50% acetonitrile in H₂O with 0.1% NH₃.H₂O) to give compound A129 (10 mg, 21% yield) as a white solid. LC/MS (ESI) m/z: 358 (M+H)⁺. ¹H NMR (400 MHz, DMSO-d6) δ 8.56 (s, 1H), 8.16 (s, 1H), 7.62 (d, J=8.8 Hz, 1H), 7.45-7.37 (m, 2H), 7.14-7.06 (m, 1H), 6.85 (d, J=8.4 Hz, 1H).

The compounds in the table below were prepared from the appropriate starting materials described previously or commercially available using the above general procedure 17.

| Structure | Name |
|---|---|
| 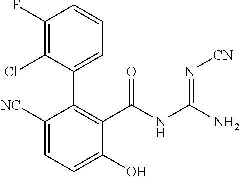 [SMILES: N#C/N=C(\N)NC(=O)c1c(O)ccc(C#N)c1-c1cccc(F)c1Cl] | 2′-chloro-6-cyano-N-(N′-cyanocarbam- imidoyl)-3′-fluoro-3-hydroxy-[1,1′- biphenyl]-2-carboxamide LC/MS (ESI) m/z: 358 (M + H)⁺. 1H NMR (400 MHz, DMSO-d6) δ 8.56 (s, 1H), 8.16 (s, 1H), 7.62 (d, J = 8.8 Hz, 1H), 7.45-7.37 (m, 2H), 7.14-7.06 (m, 1H), 6.85 (d, J = 8.4 Hz, 1H). |
| 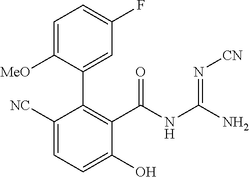 [SMILES: COc1ccc(F)cc1-c1c(C#N)ccc(O)c1C(=O)N/C(N)=N/C#N] | 6-cyano-N-(N′-cyanocarbamimidoyl)- 5′-fluoro-3-hydroxy-2′-methoxy-[1,1′- biphenyl]-2-carboxamide LC-MS: m/z 354 (M + H)⁺. 1H NMR (400 MHz, DMSO) δ 8.65 (s, 1H), 8.02 (s, 1H), 7.48 (d, J = 8.8 Hz, 1H), 7.12 (td, J = 8.8, 3.2 Hz, 1H), 7.01 (dd, J = 9.2, 4.8 Hz, 1H), 6.88 (dd, J = 8.8, 3.2 Hz, 1H), 6.71 (d, J = 8.4 Hz, 1H), 3.65 (s, 3H). |

### General Procedure 18

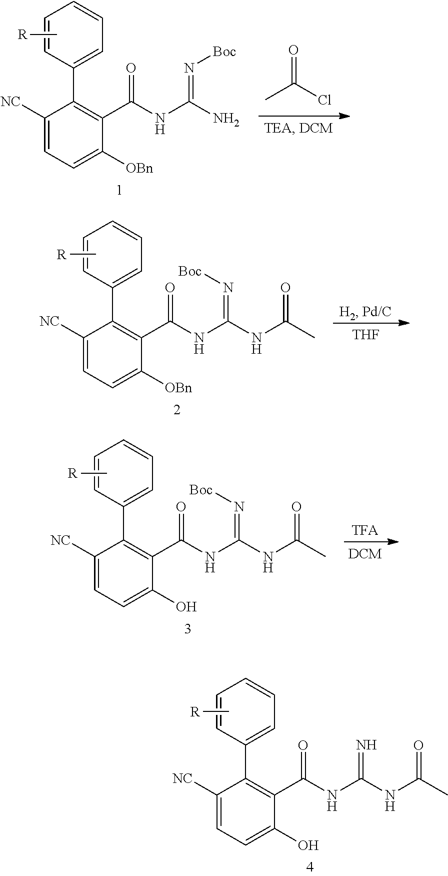

Intermediate 1 was prepared following procedure 5 and 7

### Step 1: Synthesis of Compound 2

TEA (1.5 eq) was added to a solution of compound 1 (1 eq) in DCM followed by dropwise addition of acetyl chloride (1.2 eq) at −20-10° C. under N₂ atmosphere. The resulting mixture was stirred at room temperature for 30 minutes. After the reaction was completed, the mixture was diluted with DCM and washed with H₂O and brine, dried over Na₂SO₄, filtered and concentrated under vacuum. The residue was purified by column chromatography on silica gel (eluted with Petroleum Ether:DCM=100:0 to 1:3) to give compound 2.

### Step 2: Synthesis of Compound 3

10% Pd/C (0.1 eq) was added to a solution of compound 2 (1 eq) in THF and the resulting mixture was degassed for three times under N₂ atmosphere and stirred under 15 psi H₂ for 30 minutes. The mixture was then filtered and the filtrate was concentrated under vacuum to give compound 3 without any further purification.

### Step 3: Synthesis of Compound 4

TFA (1:2, v/v) was added at 0° C. under N₂ atmosphere to a solution of compound 3 in DCM and the reaction was stirred at room temperature for 2 hours. The mixture was then concentrated under vacuum and the residue was purified via prep-HPLC (C18, 0% to 50% acetonitrile in H₂O with 0.1% NH₃.H₂O) to give compound 4.

### Synthesis of Compound A378

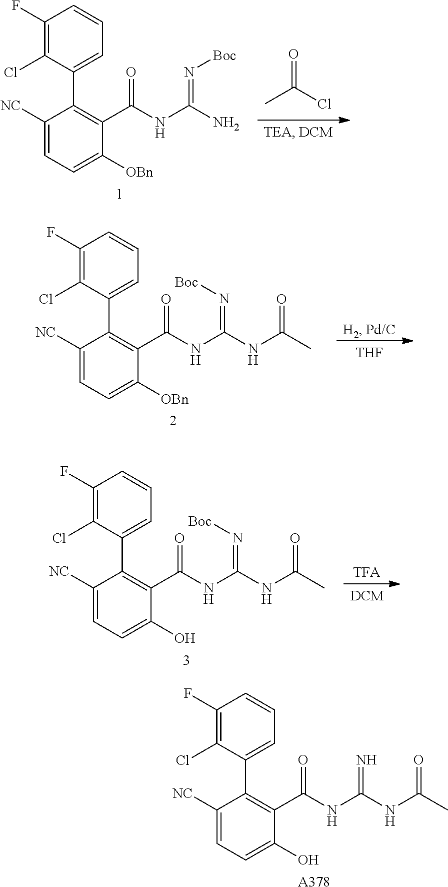

### Step 1: Synthesis of Compound A378-2

TEA (17.41 mg, 0.17 mmol) was added to a solution of compound A378-1 (60 mg, 0.114 mmol) in DCM (3 mL) followed by dropwise addition at −20-10° C. under N₂ atmosphere of acetyl chloride (10 mg, 0.126 mmol). The resulting mixture was stirred at room temperature for 30 minutes. After the reaction was completed, the mixture was diluted with DCM and washed with H₂O and brine, dried over Na₂SO₄, filtered and concentrated under vacuum. The residue was purified by column chromatography on silica gel (eluted with Petroleum Ether:DCM=100:0 to 1:3) to give compound A378-2 (32.0 mg, 49% yield) as a white solid. LC/MS (ESI) m/z: 565(M+H)⁺.

### Step 2: Synthesis of Compound A378-3

10% Pd/C (16.0 mg) was added to a solution of compound A378-2 (32.0 mg, 0.057 mmol) in THF (3 mL) and the resulting mixture was degassed for three times under N₂ atmosphere and stirred under 15 psi H₂ for 30 minutes. The mixture was then filtered and the filtrate was concentrated under vacuum to give compound A378-3 (26.9 mg, 99% yield) as a white solid without any further purification. LC/MS (ESI) m/z: 475(M+H)⁺.

### Step 5: Synthesis of Compound A378

TFA (2 mL) was added at 0° C. under N₂ atmosphere to a solution of compound A378-3 (26.9 mg, 0.056 mmol) in DCM (4 mL). The resulting mixture was stirred at room temperature for 2 hours. The mixture was then concentrated under vacuum and the residue was purified via prep-HPLC (C18, 0% to 50% acetonitrile in H₂O with 0.1% NH₃.H₂O) to afford compound A378 (4.5 mg, 21% yield) as a white solid. LC/MS (ESI) m/z: 375(M+H)⁺. ¹H NMR (400 MHz, DMSO-d6) δ 14.96 (s, 1H), 11.38 (s, 1H), 9.79 (s, 1H), 9.39 (s, 1H), 7.85 (d, J=8.7 Hz, 1H), 7.42-7.37 (m, 2H), 7.14-7.09 (m, 2H), 2.16 (s, 3H).

The compounds in the table below were prepared from the appropriate starting materials described previously or commercially available using the above general procedure 18.

| Structure | Name |
|---|---|
| 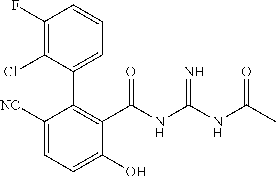 [SMILES: CC(=O)NC(=N)NC(=O)c1c(O)ccc(C#N)c1-c1cccc(F)c1Cl] | N-(N-acetylcarbamimidoyl)-2′- chloro-6-cyano-3′-fluoro-3- hydroxy-[1,1′-biphenyl]-2- carboxamide LC/MS (ESI) m/z: 375(M + H)⁺. 1H NMR (400 MHz, DMSO- d6) δ 14.96 (s, 1H), 11.38 (s, 1H), 9.79 (s, 1H), 9.39 (s, 1H), 7.85 (d, J = 8.7 Hz, 1H), 7.42- 7.37 (m, 2H), 7.14-7.09 (m, 2H), 2.16 (s, 3H). |
| 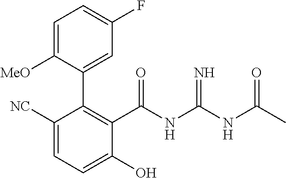 [SMILES: COc1ccc(F)cc1-c1c(C#N)ccc(O)c1C(=O)NC(=N)NC(C)=O] | N-(N-acetylcarbamimidoyl)-6- cyano-5′-fluoro-3-hydroxy-2′- methoxy-[1,1′-biphenyl]-2- carboxamide LC-MS: m/z 371 (M + H)⁺. 1H NMR (400 MHz, DMSO- d6) δ 9.60 (s, 1H), 9.42 (s, 1H), 7.62 (d, J = 8.6 Hz, 1H), 7.13- 7.08 (m, 1H), 7.01 (dd, J = 9.1, 4.6 Hz, 1H), 6.95-6.84 (m, 2H), 3.64 (s, 3H), 2.09 (s, 3H). |
| 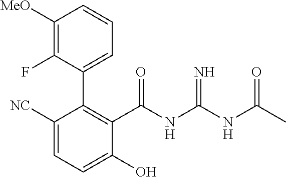 [SMILES: COc1cccc(-c2c(C#N)ccc(O)c2C(=O)NC(=N)NC(C)=O)c1F] | N-(N-acetylcarbamimidoyl)-6- cyano-2′-fluoro-3-hydroxy-3′- methoxy-[1,1′-biphenyl]-2- carboxamide LC/MS (ESI) m/z: 371(M + H)⁺. 1H NMR (400 MHz, DMSO- d6) δ 15.12 (s, 1H), 9.57 (s, 1H), 9.29 (s, 1H), 7.45 (d, J = 8.9 Hz, 1H), 7.08 (dd, J = 5.4, 2.4 Hz, 2H), 6.71-6.68 (m, 2H), 3.85 (s, 3H), 1.99 (s, 3H). |
| 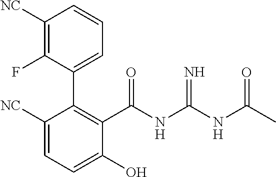 [SMILES: CC(=O)NC(=N)NC(=O)c1c(O)ccc(C#N)c1-c1cccc(C#N)c1F] | N-(N-acetylcarbamimidoyl)- 3′,6-dicyano-2′-fluoro-3- hydroxy-[1,1′-biphenyl]-2- carboxamide LC/MS (ESI) m/z: 371(M + H)⁺. LC/MS (ESI) m/z: 366 (M + H)⁺. 1H NMR (400 MHz, DMSO-d6) δ 14.98 (s, 1H), 9.48 (s, 1H), 7.90 (dd, J = 8.0, 8.0 Hz, 1H), 7.70 (d, J = 8.0 Hz, 1H), 7.63 (dd, J = 8.0, 4.0 Hz, 1H), 7.45 |
| | (dd, J = 8.0, 4.0 Hz, 1H), 6.96 |
| | (d, J = 8.0 Hz, 1H), 2.09 (s, 3H). |

### General Procedure 19

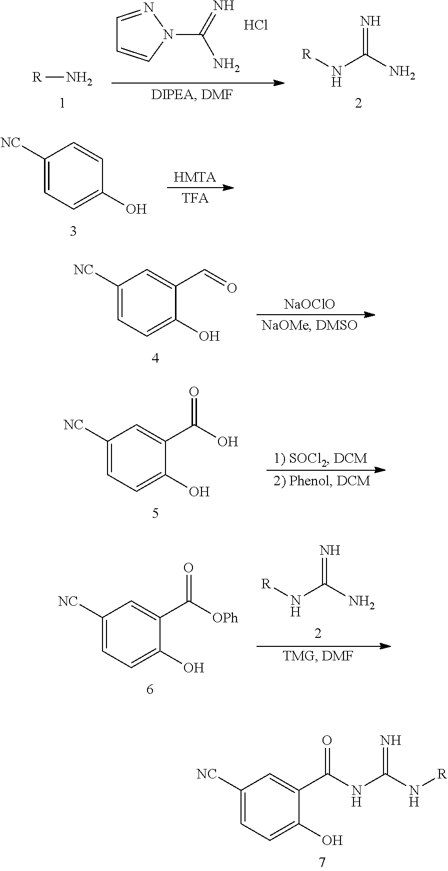

### Step 1: Synthesis of Compound 2

1H-pyrazole-1-carboxamidine hydrochloride (2 eq.) was added to a solution of the appropriate amine 1 (1 eq.) and DIPEA (8 eq.) in anhydrous DMF. The reaction mixture was stirred at room temperature overnight. MTBE was added to the reaction mixture and the mixture was stirred at room temperature for 20 minutes. The solvent was then removed and this procedure was repeated three to five times until the LC/MS showed a clean product. The appropriate alkylated acylguanidines 2 (10-20% yield) was obtained as a colorless oil or a white solid.

### Step 2: Synthesis of Compound 4

To a stirred solution of compound 3 (1 eq.) in TFA was added HMTA (2 eq.), and the resulting mixture was stirred at 100° C. for 6 hours. The mixture was then cooled to 0° C. and diluted sulfuric acid (10 mL of 98% H₂SO₄ in 130 mL of water) was added. The resulting mixture was stirred at room temperature for 30 minutes and extracted with DCM twice. The combined organic layers were washed with brine, dried over anhydrous Na₂SO₄, filtered and concentrated under vacuum. The residue was purified by column chromatography on silica gel (eluted with Petroleum Ether:EtOAc=10:1 to 3:1) to give compound 4 as a colorless oil. LC/MS (ESI) m/z: 148 (M+H)⁺.

### Step 3: Synthesis of Compound 5

NaOMe (1.1 eq.) was added to a stirred solution of compound 4 (1 eq.) in DMSO followed by portion-wise addition of NaOClO (2.2 eq.). The resulting mixture was stirred at room temperature for 4 hours. The mixture was then cooled to 0° C. and water was added slowly. The mixture was then acidified with 1 N aq. HCl solution to pH=2 and the mixture was extracted with EtOAc twice. The combined organic layers were washed with brine, dried over anhydrous Na₂SO₄, filtered and concentrated under vacuum. The residue was purified by column chromatography on silica gel (eluted with DCM:MeOH=100:1 to 20:1) to give compound 5 as a white solid. LC/MS (ESI) m/z: 162(M−H)⁻.

### Step 4: Synthesis of Compound 6

Thionyl chloride (5 eq.) was added to a suspension of compound 5 (1 eq.) in anhydrous DCM, and the resulting mixture was stirred at 50° C. for 4-6 hours. After the reaction was completed, the mixture was concentrated under vacuum. The residue was then dissolved in anhydrous DCM and a solution of phenol (1 eq.) and Et₃N (3 eq.) in dry DCM was added. The resulting mixture was stirred at room temperature for 1 hour and then quenched with saturated aq. NaHCO₃ solution. The mixture was extracted with DCM twice and the combined organic layers were washed with brine, dried over anhydrous Na₂SO₄, filtered and concentrated under vacuum. The residue was purified by column chromatography on silica gel (eluted with Petroleum Ether:EtOAc=100:0 to 40:1) to give compound 6 as a white solid. LC/MS (ESI) m/z: 238(M−H)⁻.

### Step 5: Synthesis of Compound 7

Compound 2 (2 eq.) was added to a mixture of compound 6 (1 eq.) and 1,1,3,3-Tetramethylgyanidine (1.5 eq.) in DMF, and the resulting mixture was stirred at room temperature for 16 hours. The mixture was then diluted with water and extracted with EtOAc twice. The combined organic layers were washed with brine, dried over anhydrous Na₂SO₄, filtered and concentrated under vacuum. The residue was purified via prep-TLC to give compound 7.

### Synthesis of Compound F1

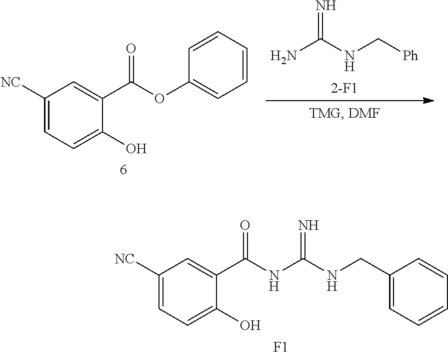

Compound 2-F1 (298 mg, 2.0 mmol) was added to a mixture of compound 6 (239 mg, 1 mmol) and 1,1,3,3-Tetramethylgyanidine (172 mg, 1.5 mmol) in DMF (5 mL), and the resulting mixture was stirred at room temperature for 16 hours. The mixture was then diluted with water and extracted with EtOAc twice. The combined organic layers were washed with brine, dried over anhydrous Na₂SO₄, filtered and concentrated under vacuum. The residue was purified via prep-TLC to give compound F1 (60 mg, 20% yield) as a white solid. LC/MS (ESI) m/z: 295(M+H)⁺. ¹H NMR (400 MHz, MeOD) δ 8.20 (s, 1H), 7.55 (d, J=8.8 Hz, 1H), 7.43-7.27 (m, 5H), 6.86 (d, J=8.7 Hz, 1H), 4.51 (s, 2H).

The compounds in the table below were prepared from the appropriate starting materials described previously or commercially available using the above general procedure 19.

| Structure | Name |
|---|---|
| 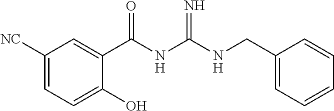 [SMILES: N#Cc1ccc(O)c(C(=O)NC(=N)NCc2ccccc2)c1] | N-(N-benzylcarbamimidoyl)-5- cyano-2-hydroxybenzamide LC-MS: m/z 295 (M + H)⁺. 1H NMR (400 MHz, MeOD) δ 8.20 (s, 1H), 7.55 (d, J = 8.8 Hz, 1H), 7.43-7.27 (m, 5H), 6.86 (d, J = 8.7 Hz, 1H), 4.51 (s, 2H). |
| 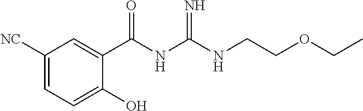 [SMILES: CCOCCNC(=N)NC(=O)c1cc(C#N)ccc1O] | 5-cyano-N-(N-(2-ethoxyethyl)- carbamimidoyl)-2-hydroxy- benzamide LC-MS: m/z 277 (M + H)⁺. 1H NMR (400 MHz, DMSO-d6) δ 16.16 (s, 1H), 8.11 (s, 1H), 7.66 (d, J = 8.4 Hz, 1H), 6.88 |
| | (d, J = 8.6 Hz, 1H), 3.53 (t, J = |
| | 5.3 Hz, 2H), 3.49 (q, J = 7.0 Hz, |
| | 2H), 3.38 (t, J = 5.3 Hz, 2H), |
| | 1.14 (t, J = 7.0 Hz, 3H). |
| 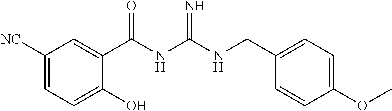 [SMILES: COc1ccc(CNC(=N)NC(=O)c2cc(C#N)ccc2O)cc1] | 5-cyano-2-hydroxy-N-(N- (4-methoxybenzyl)carbam- imidoyl)benzamide LC-MS: m/z 325 (M + H)⁺. 1H NMR (400 MHz, DMSO-d6) δ 16.08 (s, 1H), 9.63 (s, 1H), 8.80 (s, 1H), 8.11 (s, 1H), 7.66 |
| | (d, J = 8.4 Hz, 1H), 7.30 (d, J = |
| | 8.3 Hz, 2H), 6.94 (d, J = 8.5 Hz, |
| | 2H), 6.88 (d, J = 8.6 Hz, 1H), |
| | 4.38 (s, 2H), 3.74 (s, 3H). |
| 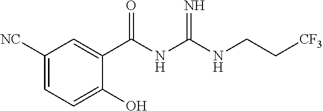 [SMILES: N#Cc1ccc(O)c(C(=O)NC(=N)NCCC(F)(F)F)c1] | 5-cyano-2-hydroxy-N-(N-(3,3,3- trifluoropropyl)carbamimidoyl)- benzamide LC-MS: m/z 301 (M + H)⁺. 1H NMR (400 MHz, DMSO-d6) δ 16.01 (s, 1H), 8.13 (s, 1H), 7.68 (d, J = 7.7 Hz, 1H), 6.89 (d, |
| | J = 7.7 Hz, 1H), 3.49 (t, J = 6.8 |
| | Hz, 2H), 2.68-2.57 (m, 2H). |
| 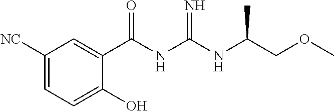 [SMILES: COC[C@H](C)NC(=N)NC(=O)c1cc(C#N)ccc1O] | (S)-5-cyano-2-hydroxy-N-(N-(1- methoxypropan-2-yl)carbam- imidoyl)benzamide LC-MS: m/z 277 (M + H)⁺. 1H NMR (400 MHz, DMSO-d6) δ 16.12 (s, 1H), 9.35 (s, 1H), 8.11 (s, 1H), 7.66 (d, J = 7.6 Hz, |
| | 1H), 6.88 (d, J = 8.5 Hz, 1H), |
| | 4.01-3.82 (m, 1H), 1.15 (d, J = |
| | 6.2 Hz, 3H). |
| 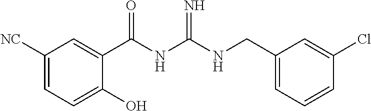 [SMILES: N#Cc1ccc(O)c(C(=O)NC(=N)NCc2cccc(Cl)c2)c1] | N-(N-(3-chlorobenzyl)carbam- imidoyl)-5-cyano-2-hydroxy- benzamide LC-MS: m/z 329 (M + H)⁺. 1H NMR (400 MHz, DMSO- d6) δ 16.01 (s, 1H), 9.64 (s, 1H), 8.90 (s, 1H), 8.14 (s, 1H), |
| | 7.88 (s, 1H), 7.68 (d, J = 8.4 |
| | Hz, 1H), 7.47-7.26 (m, 4H), |
| | 6.90 (d, J = 8.6 Hz, 1H), 4.48 |
| | (s, 2H). |
| 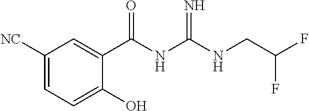 [SMILES: N#Cc1ccc(O)c(C(=O)NC(=N)NCC(F)F)c1] | 5-cyano-N-(N-(2,2-difluoro- ethyl)carbamimidoyl)-2- hydroxybenzamide LC-MS: m/z 269 (M + H)⁺. 1H NMR (400 MHz, DMSO- d6) δ 15.71 (s, 1H), 8.14 (s, 1H), 7.69 (d, J = 7.7 Hz, 1H), 6.91 (d, |
| | J = 7.7 Hz, 1H), 6.39-5.97 (m, |
| | 1H), 3.79-3.62 (m, 2H). |
| 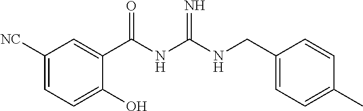 [SMILES: Cc1ccc(CNC(=N)NC(=O)c2cc(C#N)ccc2O)cc1] | 5-cyano-2-hydroxy-N-(N-(4- methylbenzyl)carbamimidoyl)- benzamide LC-MS: m/z 309 (M + H)⁺. 1H NMR (400 MHz, DMSO- d6) δ 16.07 (s, 1H), 9.66 (s, 1H), 8.82 (s, 1H), 8.11 (s, 1H), |
| | 7.81 (s, 1H), 7.67 (d, J = 8.6 |
| | Hz, 1H), 7.22 (dd, J = 14.5 Hz, |
| | 4H), 6.89 (d, J = 8.6 Hz, 1H), |
| | 4.41 (d, J = 4.0 Hz, 2H), 2.29 |
| | (s, 3H). |
| 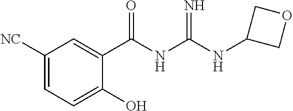 [SMILES: N#Cc1ccc(O)c(C(=O)NC(=N)NC2COC2)c1] | 5-cyano-2-hydroxy-N-(N- oxetan-3-ylcarbamimidoyl)- benzamide LC-MS: m/z 261 (M + H)⁺. 1H NMR (400 MHz, DMSO- d6) δ 15.84 (s, 1H), 8.14 (d, J = 2.2 Hz, 1H), 7.70 (d, J = |
| | 7.5 Hz, 1H), 6.92 (d, J = 8.6 |
| | Hz, 1H), 4.85-4.79 (m, 2H), |
| | 4.79-4.72 (m, 1H), 4.52 (s, |
| | 2H). |
| 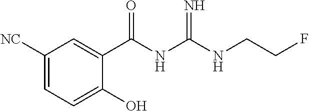 [SMILES: N#Cc1ccc(O)c(C(=O)NC(=N)NCCF)c1] | 5-cyano-N-(N-(2-fluoroethyl)- carbamimidoyl)-2-hydroxy- benzamide LC-MS: m/z 251 (M + H)⁺. 1H NMR (400 MHz, DMSO- d6) δ 16.03 (s, 1H), 8.13 (s, 1H), 7.68 (d, J = 8.4 Hz, 1H), |
| | 6.90 (d, J = 8.7 Hz, 1H), 4.63 |
| | (t, J = 4.8 Hz, 1H), 4.51 (t, |
| | J = 4.8 Hz, 1H), 3.59 (t, J = |
| | 4.9 Hz, 1H), 3.52 (t, J = 4.9 |
| | Hz, 1H). |
| 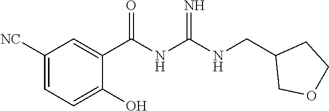 [SMILES: N#Cc1ccc(O)c(C(=O)NC(=N)NCC2CCOC2)c1] | 5-cyano-2-hydroxy-N-(N- ((tetrahydrofuran-3-yl)- methyl)carbamimidoyl)- benzamide LC-MS: m/z 289 (M + H)⁺. 1H NMR (400 MHz, DMSO- d6) δ 16.13 (s, 1H), 8.12 (s, |
| | 1H), 7.66 (d, J = 8.4 Hz, 1H), |
| | 6.90 (d, J = 8.7 Hz, 1H), 3.79- |
| | 3.70 (m, 2H), 3.66-3.60 (m, |
| | 1H), 3.43-3.40 (m, 1H), 3.24- |
| | 3.20(m, 2H), 2.50-2.46 (m, |
| | 1H), 2.03-1.95 (m, 1H), 1.61- |
| | 1.52 (m, 1H). |
| 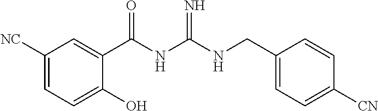 [SMILES: N#Cc1ccc(CNC(=N)NC(=O)c2cc(C#N)ccc2O)cc1] | 5-cyano-N-(N-(4-cyano- benzyl)carbamimidoyl)-2- hydroxybenzamide LC-MS: m/z 320 (M + H)⁺. 1H NMR (400 MHz, DMSO- d6) δ 15.56 (s, 1H), 8.50 (br s, 2H), 8.09 (s, 1H), 7.84 (d, |
| | J = 8.2 Hz, 2H), 7.58 (d, J = |
| | 8.6 Hz, 1H), 7.53 (d, J = 8.3 |
| | Hz, 2H), 6.78 (d, J = 8.5 Hz, |
| | 1H), 4.52 (s, 2H). |
| 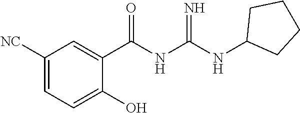 [SMILES: N#Cc1ccc(O)c(C(=O)NC(=N)NC2CCCC2)c1] | 5-cyano-N-(N-cyclopentyl- carbamimidoyl)-2-hydroxy- benzamide LC-MS: m/z 287 (M + H)⁺. 1H NMR (400 MHz, DMSO- d6) δ 16.11 (s, 1H), 9.28 (d, J = 7.1 Hz, 1H), 8.84 (s, 1H), |
| | 8.11 (s, 1H), 7.79 (s, 1H), |
| | 7.65 (d, J = 8.4 Hz, 1H), 6.87 |
| | (d, J = 8.5 Hz, 1H), 4.03-3.84 |
| | (m, 1H), 2.06-1.89 (m, 2H), |
| | 1.77-1.64 (m, 2H), 1.62-1.43 |
| | (m, 4H). |
| 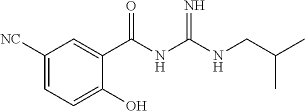 [SMILES: CC(C)CNC(=N)NC(=O)c1cc(C#N)ccc1O] | 5-cyano-2-hydroxy-N-(N- isobutylcarbamimidoyl)- benzamide LC-MS: m/z 261 (M + H)⁺. 1H NMR (400 MHz, DMSO- d6) δ 16.20 (s, 1H), 9.43 (s, 1H), 8.12 (d, J = 2.0 Hz, 1H), |
| | 7.65 (dd, J = 8.7, 2.1 Hz, 1H), |
| | 6.87 (d, J = 8.6 Hz, 1H), 3.04 |
| | (t, J = 6.4 Hz, 2H), 1.90-1.79 |
| | (m, 1H), 0.92 (d, J = 6.7 hz, |
| | 6H). |

### Synthesis of A92

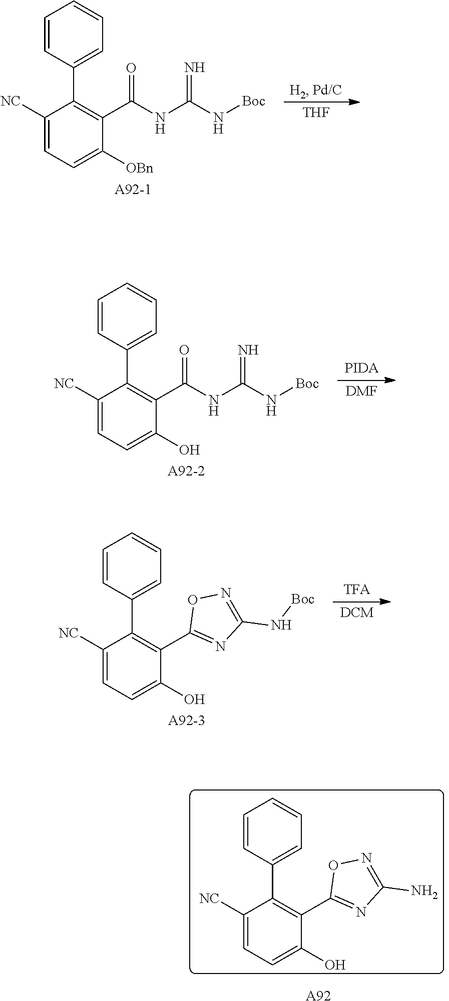

Intermediate A92-1 was prepared following general procedures 5 and 7.

### Step 1: Synthesis of Compound A92-2

10% Pd/C (42 mg) was added under N₂ atmosphere to a solution of A92-1 (141 mg, 0.3 mmol) in THF (8 mL), and the resulting mixture was stirred at room temperature for 30 minutes under 15 psi H₂. The mixture was then filtered and the filtrate was concentrated under vacuum to give A92-2 (98 mg, 86% yield) as a white solid without any further purification. LC-MS: m/z 381 (M+H)⁺.

### Step 2: Synthesis of Compound A92-3

(diacetoxyiodo)benzene (166 mg, 0.52 mmol) was added at 0° C. under N₂ atmosphere to a solution of A92-2 (98 mg, 0.26 mmol) in DMF (8 mL), and the resulting mixture was stirred at room temperature for 6 hours. The mixture was then diluted with EtOAc and washed with saturated aq. NH₄Cl solution. The organic layer was then dried over Na₂SO₄, filtered and concentrated under vacuum. The residue was purified by column chromatography on silica gel (eluted with Petroleum Ether:EtOAc=50:1 to 4:1) to give compound A92-3 (52 mg, 53% yield) as a yellow oil. LC/MS (ESI) m/z: 379(M+H)⁺.

### Step 3: Synthesis of Compound A92

TFA (1 mL) was added dropwise at 0° C. under N₂ atmosphere to a solution of compound A92-3 (52 mg, 0.14 mmol) in anhydrous DCM (2 mL), and the resulting mixture was stirred at room temperature for 3 hours. The mixture was then concentrated under vacuum and the residue was purified via prep-HPLC (C18, 0% to 50% acetonitrile in H₂O with 0.1% NH₃.H₂O) to give compound A92 (13 mg, 33% yield) as a white solid. LC/MS (ESI) m/z: 279(M+H)⁺. 1H NMR (400 MHz, DMSO-d6) δ 7.70 (d, J=8.6 Hz, 1H), 7.40-7.31 (m, 3H), 7.26-7.16 (m, 2H), 6.83 (d, J=8.1 Hz, 1H), 6.06 (s, 2H).

### Synthesis of A133

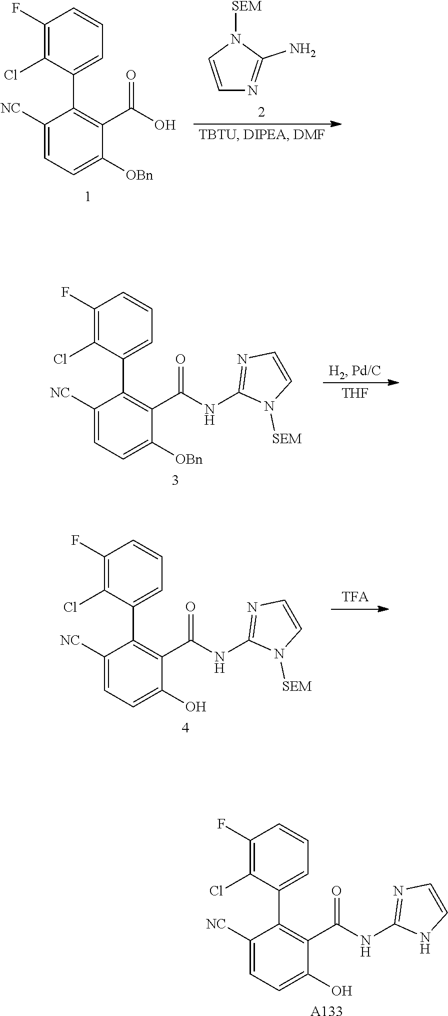

### Step 1: Synthesis of Compound A133-3

DIPEA (108 mg, 0.84 mmol) and TBTU (81 mg, 0.25 mmol) were added at room temperature to a mixture of compound A133-1 (80 mg, 0.21 mmol) and A133-2 (54 mg, 0.25 mmol) in DMF (3 mL), and the resulting mixture was stirred at 30° C. for 16 hours. The mixture was then diluted with water and extracted with EtOAc twice. The combined organic layers were washed with brine, dried over anhydrous Na₂SO₄, filtered and concentrated under vacuum. The residue was purified by column chromatography on silica gel (eluted with Petroleum Ether:EtOAc=8:1 to 3:1) to give compound A133-3 (30 mg, 25% yield) as a light yellow solid. LC/MS (ESI) m/z: 577 (M+H)⁺.

### Step 2: Synthesis of Compound A133-4

10% Pd/C (30 mg) was added at 0° C. to a solution of compound A133-3 (30 mg, 0.05 mmol) in THF (4 mL), and the mixture was degassed under N₂ atmosphere for three times and stirred under a H₂ at room temperature for 30 minutes. The mixture was then filtered and the filtrate was concentrated under vacuum. The residue was purified by column chromatography on silica gel (eluted with Petroleum Ether:EtOAc=10:1 to 4:1) to give compound A133-4 (15 mg, 59% yield) as a white solid. LC/MS (ESI) m/z: 487 (M+H)⁺.

### Step 3: Synthesis of Compound A133

Compound A133-4 (15 mg, 0.03 mmol) was dissolved in TFA (2 mL) and reaction stirred at room temperature for 1 hour. The reaction mixture was then concentrated under vacuum and the residue was dissolved in saturated aq. NaHCO₃ solution and extracted with EtOAc twice. The combined organic layers were washed with brine, dried over anhydrous Na₂SO₄, filtered and concentrated under vacuum. The residue was purified by prep-HPLC (C18, 0% to 50% acetonitrile in H₂O with 0.1% NH₃.H₂O) to give compound A133 (3 mg, 27% yield) as a white solid. LC/MS (ESI) m/z: 357 (M+H)⁺. ¹H NMR (400 MHz, DMSO-d6) δ 12.35 (br s, 2H), 7.72 (d, J=8.8 Hz, 1H), 7.44-7.32 (m, 2H), 7.10 (d, J=6.4 Hz, 1H), 7.00 (d, J=8.4 Hz, 1H), 6.87 (s, 2H).

### Synthesis of A141

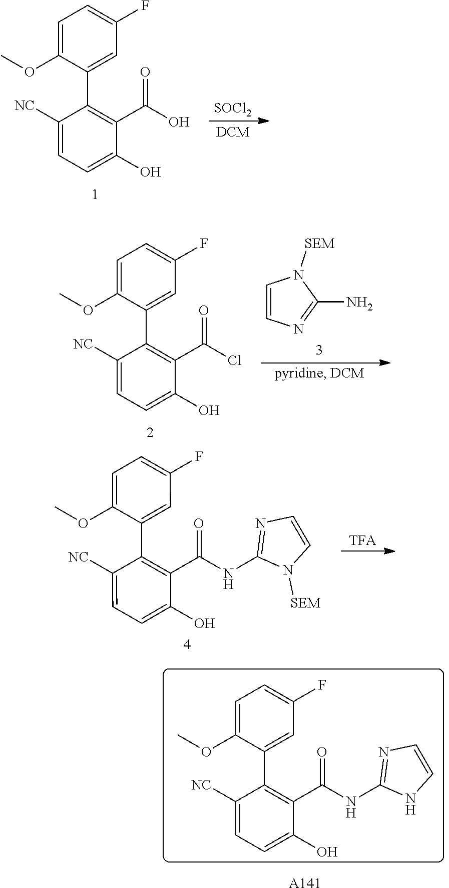

### Step 1: Synthesis of Compound A141-2

Thionyl chloride (0.51 mL, 7.0 mmol) was added at 0° C. to a solution of compound A141-1 (200 mg, 0.70 mmol) in DCM (5 mL), and the resulting mixture was stirred at 65° C. for 2 hours. The mixture was then concentrated under vacuum to give compound A141-2 (200 mg, 99% yield) as a light yellow solid which was used in the next step without any further purification.

### Step 2: Synthesis of Compound A141-4

Anhydrous pyridine (0.28 mL, 3.48 mmol) was added at 0° C. under N₂ atmosphere to a solution of A141-3 (446 mg, 2.09 mmol) in anhydrous DCM (5 mL), and the reaction was stirred at 0° C. for 30 minutes. A solution of A141-2 (200 mg, 0.66 mmol) in DCM (5 mL) was added dropwise to the above reaction and the resulting mixture was stirred at room temperature overnight under N₂ atmosphere. The mixture was then concentrated under vacuum and the residue was purified by column chromatography on silica gel (eluted with Petroleum Ether:EtOAc=30:1 to 2:1) to give A141-4 (60 mg, 18%) as a yellow oil. LC/MS (ESI) m/z: 483(M+H)⁺.

### Step 3: Synthesis of Compound A141

A141-4 (60 mg, 0.12 mmol) was dissolved at 0° C. in TFA (2 mL), and the reaction was stirred at 40° C. for 3 hours. The mixture was then concentrated under vacuum and the residue was dissolved in EtOAc, basified with saturated aq. NaHCO₃ solution and extracted with EtOAc twice. The combined organic layers were washed with brine, dried over anhydrous Na₂SO₄, filtered and concentrated under vacuum. The residue was purified via prep-HPLC (C18, 0% to 50% acetonitrile in H₂O with 0.1% NH₃.H₂O) to give A141 (15 mg, 34%) as a white solid. LC/MS (ESI) m/z: 353(M+H)⁺. ¹H NMR (400 MHz, DMSO-d6) δ 12.33 (br s, 2H), 7.68 (d, J=8.8 Hz, 1H), 7.15-7.09 (m, 1H), 7.03 (dd, J=8.8, 4.4 Hz, 1H), 6.99-6.92 (m, 2H), 6.85 (s, 2H), 3.65 (s, 3H).

### Synthesis of A156

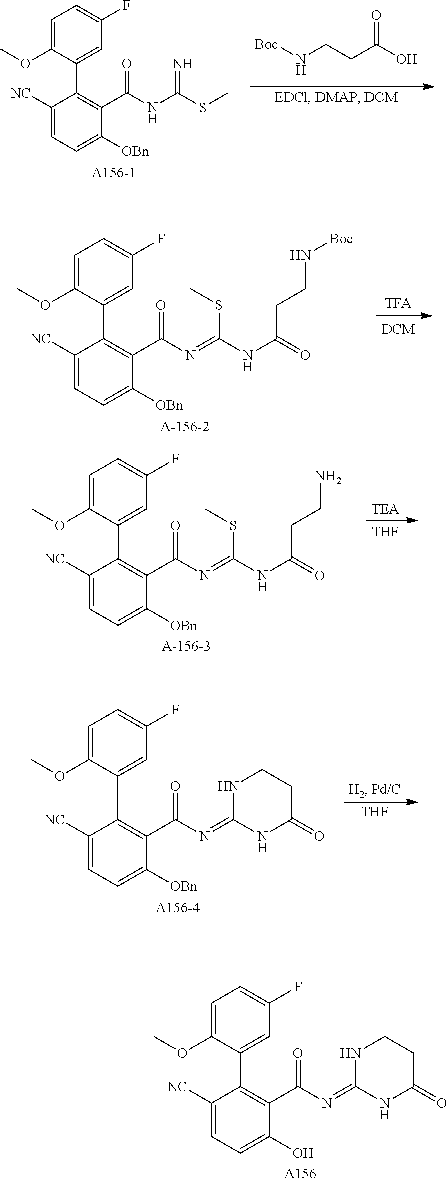

### Step 1: Synthesis of Compound A156-2

EDCI (104 mg, 0.67 mmol) and DMAP (163 mg, 1.33 mmol) were added to a mixture of compound A156-1 (200 mg, 0.45 mmol) and Boc-beta-alanine (84 mg, 0.45 mmol) in DCM (10 mL), and the resulting mixture was stirred at 35° C. for 24 hours. The mixture was then diluted with water and extracted with DCM twice. The combined organic layers were washed with brine, dried over anhydrous Na₂SO₄, filtered and concentrated under vacuum. The residue was purified by flash column chromatography on silica gel (Petroleum Ether:EtOAc=10:1 to 2:1) to give compound A156-2 (210 mg, 76% yield) as a light yellow solid. LC/MS (ESI) m/z: 621 (M+H)⁺.

### Step 2: Synthesis of Compound A156-3

TFA (3 mL) was added at 0° C. to a solution of compound A156-2 (210 mg, 0.34 mmol) in DCM (3 mL) and the reaction was stirred at room temperature for 1 hour. The mixture was then concentrated under vacuum to give compound A156-3 (176 mg, 99% yield) as a light yellow solid without any further purification. LC/MS (ESI) m/z: 521 (M+H)⁺.

### Step 3: Synthesis of Compound A156-4

TEA (0.19 mL, 1.39 mmol) was added to a solution of compound A156-3 (176 mg, 0.35 mmol) in THF (5 mL) and the reaction mixture was stirred at room temperature for 30 minutes. The mixture was then concentrated under vacuum and the residue was purified by flash column chromatography on silica gel (DCM:MeOH=200:1 to 100:1) to give compound A156-4 (84 mg, 51% yield) as a white solid. LC/MS (ESI) m/z: 473 (M+H)⁺.

### Step 4: Synthesis of Compound A156

10% Pd/C (60 mg) was added to a solution of compound A156-4 (60 mg, 0.15 mmol) in THF (5 mL) and the reaction mixture was degassed under N₂ atmosphere for three times and stirred under H₂ at room temperature for 30 minutes. The mixture was then filtered and the filtrate was concentrated under vacuum. The residue was purified via prep-HPLC (C18, 0% to 50% acetonitrile in H₂O with 0.1% NH₃.H₂O) to give compound A156 (16 mg, 24% yield) as a white solid. LC/MS (ESI) m/z: 383 (M+H)⁺. ¹H NMR (400 MHz, DMSO-d6) δ 14.71 (s, 1H), 11.31 (s, 1H), 10.18 (s, 1H), 7.73 (d, J=8.7 Hz, 1H), 7.12 (td, J=8.7, 3.1 Hz, 1H), 7.05-6.97 (m, 2H), 6.92 (dd, J=8.8, 3.1 Hz, 1H), 3.65 (s, 3H), 3.53 (t, J=7.2 Hz, 2H), 2.59 (t, J=7.2 Hz, 2H).

### Synthesis of A162 and A163

### Synthesis of Key Intermediate 7

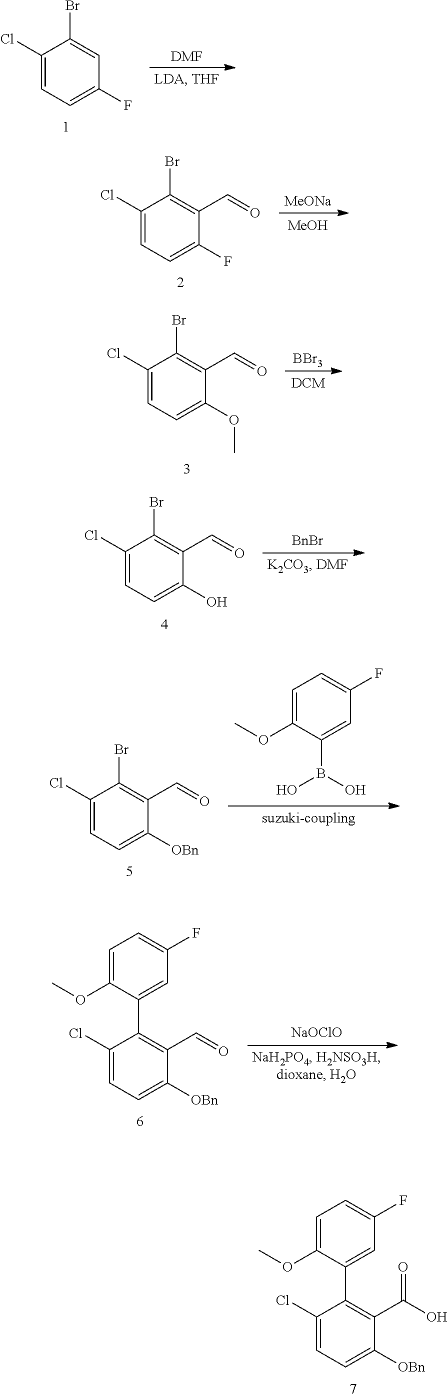

### Step 1: Synthesis of Compound 2

LDA (4.3 mL, 2.0 M in THF) was added under N₂ atmosphere at −78° C. to a solution of 1 (1.5 g, 7.2 mmol) in anhydrous THF (30 mL), and the reaction mixture was stirred at −78° C. for 2 hours before anhydrous DMF (0.6 g, 8.66 mmol) was added. The reaction was stirred −78° C. for an additional hour, and then the reaction was quenched with iced saturated aq. NH₄Cl solution and extracted with EtOAc twice. The combined organic layers were washed with brine, dried over Na₂SO₄, filtered and concentrated under vacuum. The residue was purified by flash column chromatography on silica gel (eluted with Petroleum Ether:EtOAc=20:1 to 5:1) to give 2 (1.4 g, 83% yield). LC/MS (ESI) m/z: 237 (M+H)⁺.

### Step 2: Synthesis of Compound 3

Sodium methoxide (1.27 g, 23.59 mmol) was added to a solution of compound 2 (1.4 g, 5.9 mmol) in MeOH (5 mL). The resulting mixture was heated to 80° C. for 30 minutes, then the mixture was concentrated under vacuum. The residue was diluted with water (100 mL) and extracted with EtOAc. The organic layer was dried over anhydrous Na₂SO₄, filtered and concentrated under vacuum to give compound 3 (1.23 g, 84% yield) as a yellow solid and was used without any further purification. LC/MS (ESI) m/z: 249 (M+H)⁺.

### Step 3: Synthesis of Compound 4

Boron tribromide (26.75 mL, 48.1 mmol) was slowly added at −20° C. under N₂ atmosphere to a solution of compound 3 (1.2 g, 4.8 mmol) in dry DCM (20 mL). The resulting mixture was slowly warmed to room temperature during a period of 1 hour, and then the mixture was quenched by addition of MeOH (0.4 mL) at 0° C. and concentrated under vacuum. The residue was purified by flash column chromatography on silica gel (eluted with 0% to 20% EtOAc in Petroleum Ether) to give compound 4 (1.05 g, 93% yield) as a light yellow solid. LC/MS (ESI) m/z: 235(M+H)⁺.

### Step 4: Synthesis of Compound 5

Benzyl bromide (0.59 mL, 4.9 mmol) and potassium carbonate (0.76 mL, 13.38 mmol) were added to a solution of compound 4 (1.05 g, 4.46 mmol) in DMF (15 mL). The resulting mixture was stirred at overnight at room temperature. The reaction was then quenched by the addition of water (80 mL) and extracted with EtOAc twice. The combined organic layers were washed with brine, dried over anhydrous Na₂SO₄, filtered and concentrated under vacuum. The residue was purified by column chromatography on silica gel (eluted with Petroleum Ether:EtOAc=8:1) to give compound 5 (1.26 g, 87% yield) as a light yellow solid. LC/MS (ESI) m/z: 325 (M+H)⁺.

### Step 5: Synthesis of Compound 6

Potassium carbonate (637 mg, 4.61 mmol) was added under N₂ atmosphere to a mixture of compound 5 (500 mg, 1.54 mmol) and (5-fluoro-2-methoxyphenyl)boronic acid (392 mg, 2.3 mmol) in dioxane (25 mL) and H₂O (2.5 mL) followed by the addition of Pd(PPh₃)₄ (355 mg, 0.31 mmol). The resulting mixture was stirred at 95° C. for 15 hours under N₂ atmosphere. The reaction was then cooled to room temperature, diluted with EtOAc and washed with water. The organic layer was dried over Na₂SO₄, filtered and concentrated under vacuum. The residue was purified by flash column chromatography on silica gel (eluted with Petroleum Ether:EtOAc=20:1 to 5:1) to give compound 6 (520 mg, 91% yield). LC/MS (ESI) m/z: 371(M+H)⁺.

### Step 6: Synthesis of Compound 7

A solution of compound 6 (520 mg, 1.48 mmol) in dioxane (30 mL) was added to a mixture of NaH₂PO₄ (712 mg, 5.9 mmol) and sulfamic acid (624 mg, 2.23 mmol) in water (20 mL), followed by the addition of a solution of sodium chlorite (175 mg, 1.93 mmol) in H₂O (10 mL). The resulting mixture was stirred at 0° C. for 2 hour, and then the mixture was diluted with water and extracted with EtOAc twice. The combined organic layers were dried over anhydrous Na₂SO₄, filtered and concentrated under vacuum. The residue was purified by column chromatography on silica gel (eluted with Petroleum Ether:EtOAc=10:1 to 1:1) to give compound 7 (480 mg, 84% yield) as a light yellow solid. LC/MS (ESI) m/z: 385(M−H)⁺.

### Procedure for the Preparation of A162

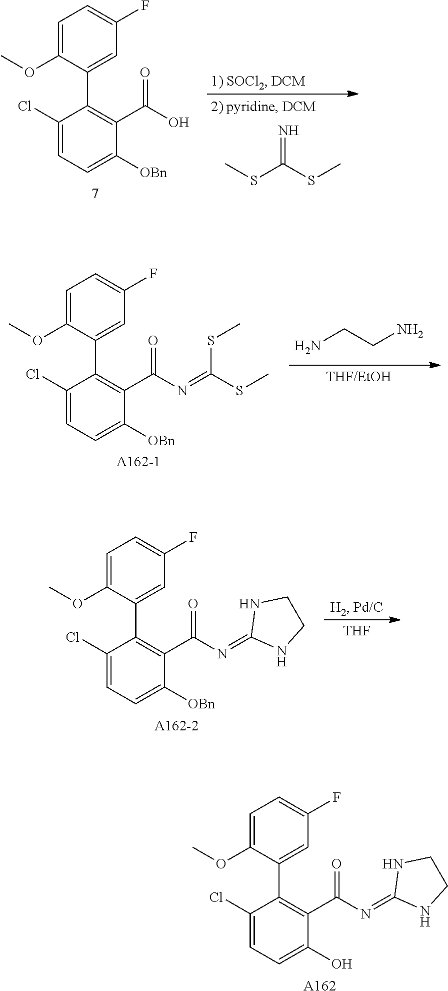

### Step 1: Synthesis of Compound A162-1

Thionyl chloride (0.3 mL, 3.9 mmol) was added to a solution of compound 7 (150 mg, 0.39 mmol) in anhydrous DCM (10 mL), and the resulting mixture was heated to 65° C. for 2 hours. The mixture was then cooled to room temperature and concentrated under vacuum. The crude acyl chloride was dissolved in anhydrous DCM (3 mL) and added dropwise at 0° C. under N₂ atmosphere to a solution of bis(methylsulfanyl)methanimine (71 mg, 0.58 mmol) and dry pyridine (1.03 g, 3.878 mmol) in anhydrous DCM (10 mL). The resulting mixture was stirred at 20° C. for 45 minutes, and then the mixture was quenched with saturated aq. NaHCO₃ solution and extracted with DCM. The organic layer was dried over anhydrous Na₂SO₄, filtered and concentrated under vacuum. The residue was purified by column chromatography on silica gel (eluted with Petroleum Ether:EtOAc=10:1 to 1:1) to give compound A162-1 (154 mg, 81% yield) as a light yellow solid. LC/MS (ESI) m/z: 490(M+H)⁺.

### Step 2: Synthesis of Compound A162-2

Ethane-1,2-diamine (0.09 mL, 1.23 mmol) was added to a solution of A162-1 (150 mg, 0.31 mmol) in THF (3 mL) and EtOH (3 mL). The reaction mixture was heated to 80° C. for 1 hour. The mixture was then concentrated under vacuum and the residue was recrystallized from MTBE (4 mL) to give A162-2 (110 mg, 79%) as a white solid. LC/MS (ESI) m/z: 454(M+H)⁺.

### Step 3: Synthesis of Compound A162

10% Pd/C (50 mg) was added under N₂ atmosphere to a solution of A162-2 (100 mg, 0.22 mmol) in THF (8 mL), and the resulting mixture was stirred at room temperature for 25 minutes under 15 psi H₂. The mixture was then filtered and the filtrate was concentrated under vacuum. The residue was purified via prep-HPLC (C18, 0% to 50% acetonitrile in H₂O with 0.1% NH₃.H₂O) to give A162 (25 mg, 31% yield) as a white solid. LC/MS (ESI) m/z: 364(M+H)⁺. 1H NMR (400 MHz, DMSO-d6) δ: 15.43 (s, 1H), 8.34 (s, 2H), 7.37 (d, J=8.8 Hz, 1H), 7.03 (td, J=8.7, 3.1 Hz, 1H), 6.95 (dd, J=9.0, 4.6 Hz, 1H), 6.86 (d, J=8.8 Hz, 1H), 6.71 (dd, J=9.0, 3.1 Hz, 1H), 3.61 (s, 3H), 3.50 (s, 4H).

### Procedure for the Preparation of A163

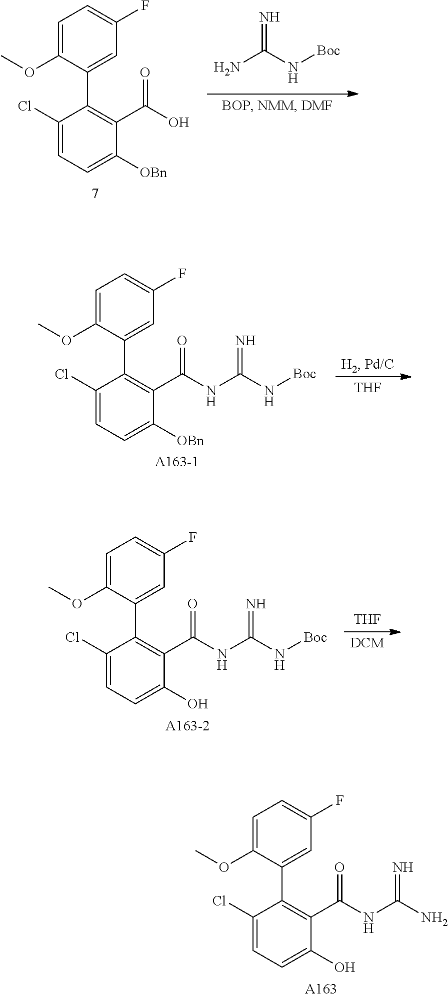

### Step 1: Synthesis of Compound A163-1

NMM (0.17 mL, 1.55 mmol) and PyBOP (161 mg, 0.362 mmol) were added under N₂ atmosphere to a solution of compound 7 (100 mg, 0.26 mmol) and tert-butyl N-carbamimidoylcarbamate (107 mg, 0.67 mmol) in DMF (12 mL). The resulting mixture was stirred at room temperature for 15 hours. The mixture was then poured into water and extracted with EtOAc twice. The combined organic layers were washed with brine, dried over Na₂SO₄, filtered and concentrated under vacuum. The residue was purified by flash column chromatography on silica gel (eluted with Petroleum Ether:EtOAc=10:1 to 3:1) to give A163-1 (81 mg, 59% yield) as a white solid. LC/MS (ESI) m/z: 528(M+H)⁺.

### Step 2: Synthesis of Compound A163-2

10% Pd/C (35 mg) was added under N₂ atmosphere to a solution of A163-1 (70 mg, 0.133 mmol) in THF (8 mL) and the resulting mixture was stirred at room temperature for 1 hour under 15 psi H₂. The mixture was then filtered and the filtrate was concentrated under vacuum to give A163-2 (51 mg, 88% yield) as a white solid without any further purification. LC/MS (ESI) m/z: 438 (M+H)⁺.

### Step 3: Synthesis of Compound A163

TFA (3 mL) was added at 0° C. to a solution of A163-2 (50 mg, 0.12 mmol) in DCM (3 mL). The resulting mixture was stirred at room temperature for 1 hour and then the mixture was concentrated under vacuum. The residue was purified via prep-HPLC (C18, 0% to 50% acetonitrile in H₂O with 0.1% NH₃.H₂O) to give A163 (12 mg, 31% yield) as a white solid. LC-MS (ESI) m/z 338 (M+H)⁺. ¹H NMR (400 MHz, DMSO-d6) δ: 15.75 (s, 1H), 8.09 (br s, 2H), 7.36 (d, J=8.8 Hz, 1H), 7.03 (td, J=8.7, 3.1 Hz, 1H), 6.94 (dd, J=9.0, 4.7 Hz, 1H), 6.83 (d, J=8.8 Hz, 1H), 6.71 (dd, J=9.0, 3.1 Hz, 1H), 3.61 (s, 3H).

### Synthesis of A161

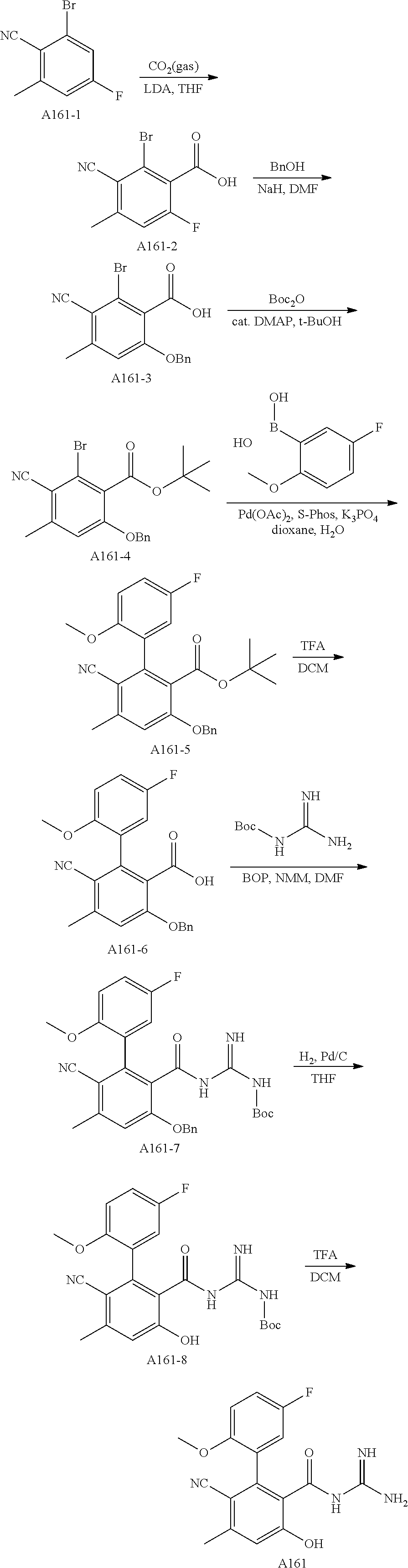

### Step 1: Synthesis of Compound A161-2

LDA (0.3 mL, 2M in THF) was added at −78° C. under N₂ atmosphere to a solution of compound A161-1 (100 mg, 0.47 mmol) in anhydrous THF (10 mL), and the resulting mixture was stirred at −78° C. for 1 hour. Then CO₂ (gas) was bubbled into the above mixture at −78° C. for 30 minutes. The mixture was quenched with ice/water and extracted with methyl tert-butyl ether twice. Then the aqueous layer was separated, acidified with 0.5 M aq. HCl solution and extracted with EtOAc twice. The combined organic layers were washed with brine, dried over Na₂SO₄, filtered and concentrated under vacuum to give compound A161-2 (80 mg, 66% yield) as a yellow solid. LC/MS (ESI) m/z: 256 (M−H)⁻.

### Step 2: Synthesis of Compound A161-3

60% NaH (26 mg, 0.65 mmol) was added at 0° C. under N₂ atmosphere to a solution of benzyl alcohol (0.05 mL, 0.47 mmol) in anhydrous DMF (5 mL). The mixture was stirred at 0° C. for 1 hour before A161-2 (80 mg, 0.31 mmol) was added. The resulting mixture was stirred at room temperature for 1.5 hours. The mixture was then quenched with ice/H₂O and extracted with Methyl tert-butyl ether twice. The aqueous layer was separated, acidified with 0.5 M aq. HCl solution and extracted with EtOAc twice. The combined organic layers were washed with brine, dried over anhydrous Na₂SO₄, filtered and concentrated under vacuum. The residue was purified by column chromatography on silica gel (eluted with DCM:MeOH=80:1 to 40:1) to give compound A161-3 (100 mg, 93% yield) as a light yellow solid. LC/MS (ESI) m/z: 344 (M−H)⁻.

### Step 3: Synthesis of Compound A161-4

di-tert-butyl dicarbonate (0.12 mL, 0.58 mmol) and DMAP (7 mg, 0.06 mmol) were added to a solution of compound A161-3 (100 mg, 0.29 mmol) in t-BuOH (10 mL), and the mixture was stirred at 60° C. for 16 hours. The mixture was then cooled to room temperature and concentrated under vacuum. The residue was purified by column chromatography on silica gel (Petroleum Ether:EtOAc=50:1 to 10:1) to give compound A161-4 (80 mg, 69% yield) as a white solid. LC/MS (ESI) m/z: 402 (M+H)⁺.

### Step 4: Synthesis of Compound A161-5

K₃PO₄ (106 mg, 0.50 mmol) and S-Phos (16 mg, 0.04 mmol) were added under N₂ atmosphere to a mixture of compound A161-4 (80 mg, 0.2 mmol) and 5-fluoro-2-methoxyphenylboronic acid (54 mg, 0.32 mmol) in dioxane (8 mL) and H₂O (1 mL) followed by the addition of Pd(OAc)₂ (9 mg, 0.04 mmol). The resulting mixture was stirred at 95° C. for 16 hours under N₂ atmosphere. The mixture was then cooled to room temperature, diluted with EtOAc and washed with water. The organic layer was then dried over Na₂SO₄, filtered and concentrated under vacuum. The residue was purified by flash column chromatography on silica gel (eluted with Petroleum Ether:EtOAc=10:1 to 5:1) to give compound A161-5 (70 mg, 79% yield) as a white solid. LC/MS (ESI) m/z: 392 (M−56+H)⁺.

### Step 5: Synthesis of Compound A161-6

TFA (2 mL) was added at 0° C. to a solution of compound A161-5 (70 mg, 0.16 mmol) in DCM (4 mL) and the reaction was stirred at room temperature for 1 hour. The mixture was then concentrated under vacuum to give compound A161-6 (60 mg, 98% yield) as a light yellow solid without any further purification. LC/MS (ESI) m/z: 390 (M−H)⁻.

### Step 6: Synthesis of Compound A161-7

4-Methylmorpholine (0.05 mL, 0.5 mmol) and PyBOP (47 mg, 0.11 mmol) were added to a mixture of compound A161-6 (30 mg, 0.08 mmol) and Boc-guanidine (230 mg, 0.52 mmol) in DMF (2 mL), and the reaction was stirred at room temperature for 16 hours. The mixture was then diluted with H₂O and extracted with EtOAc twice. The combined organic layers were washed with saturated aq. NH₄Cl solution and brine, dried over anhydrous Na₂SO₄, filtered and concentrated under vacuum. The residue was purified by column chromatography on silica gel (Petroleum Ether:EtOAc=10:1 to 2:1) to give compound A161-7 (40 mg, 98% yield) as a white solid. LC/MS (ESI) m/z: 533 (M+H)⁺.

### Step 7: Synthesis of Compound A161-8

10% Pd/C (20 mg) was added under N₂ atmosphere to a solution of compound A161-7 (40 mg, 0.08 mmol) in THF (4 mL) and the resulting mixture was degassed under N₂ atmosphere for three times and stirred under H₂ at room temperature for 30 minutes. The mixture was then filtered and the filtrate was concentrated under vacuum to give compound A161-8 (30 mg, 90% yield) as a white solid without any further purification. LC/MS (ESI) m/z: 443 (M+H)⁺.

### Step 8: Synthesis of Compound A161

TFA (3 mL) was added at 0° C. to a solution of compound A161-8 (30 mg, 0.07 mmol) in DCM (3 mL) and the reaction was stirred at room temperature for 1 hour. The mixture was then concentrated under vacuum and the residue was purified via prep-HPLC (C18, 0% to 50% acetonitrile in H₂O with 0.1% NH₃.H₂O) to give compound A161 (7.2 mg, 31% yield) as a white solid. LC/MS (ESI) m/z: 343 (M+H)⁺. ¹H NMR (400 MHz, DMSO-d6) δ 7.67 (br s, 3H), 7.09 (td, J=8.7, 3.1 Hz, 1H), 6.99 (dd, J=9.0, 4.6 Hz, 1H), 6.85 (dd, J=8.9, 3.1 Hz, 1H), 6.77 (s, 1H), 3.64 (s, 3H), 2.36 (s, 3H).

### INCORPORATION BY REFERENCE

All of the U.S. patents and U.S. and PCT patent application publications cited herein are hereby incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

We claim:

1. A compound of Formula (I) or (II): 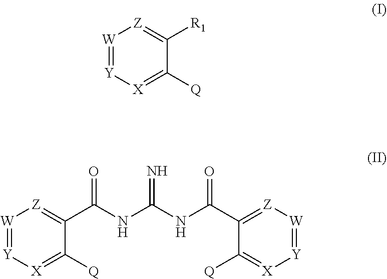
wherein
X, Y, W, and Z are independently selected from N and C(R); provided that no more than two of X, Y, W, and Z are N;
if Z and W, or W and Y, or Y and X are C(R), then any two adjacent instances of R taken together may form a fused 3-8 membered ring;
Q is OH, —NHSO₂R′, —COOH, —C(O)NHSO₂R″, —SO₂NHC(O)R″, tetrazolyl, or —CRₓR_{y}OH;
R is independently selected from H, alkyl, alkenyl, alkynyl, cycloalkyl, heteroalkyl, cycloheteroalkyl, substituted or unsubstituted aryl, substituted or unsubstituted -alkylene-aryl, substituted or unsubstituted heteroaryl, substituted or unsubstituted -alkylene-heteroaryl, haloalkyl, halocycloalkyl, halocycloheteroalkyl, —O-alkyl, —O-haloalkyl, —O-cycloalkyl, —N-alkyl, —N-haloalkyl, —N-cycloalkyl, —S-alkyl, —S-haloalkyl, —S-cycloalkyl, —O-heteroalkyl, —O-cycloheteroalkyl, —N-heteroalkyl, —N-cycloheteroalkyl, —S-heteroalkyl, —S-cycloheteroalkyl, —O-aryl, —N-aryl, —S-aryl, —O-heteroaryl, —N-heteroaryl, —S-heteroaryl, substituted or unsubstituted —O-alkylene-aryl, substituted or unsubstituted —N-alkylene-aryl, substituted or unsubstituted —S-alkylene-aryl, substituted or unsubstituted —O-alkylene-heteroaryl, substituted or unsubstituted —N-alkylene-heteroaryl, substituted or unsubstituted —S-alkylene-heteroaryl, halide, —CN, —NO₂, —S(O)Rₐ, —S(O)₂Rₐ, —C(O)Rₐ, —C(O)₂Rₐ, —C(O)NRₐR_{b}, OH, and C(O)NR′C(NR′)NRₐR_{b};
R′ is H, alkyl, or aryl;
R″ is alkyl or aryl;
Rₐ and R_{b} are independently H, alkyl, alkenyl, alkynyl, substituted or unsubstituted aryl, cycloalkyl, heteroalkyl, haloalkyl, cycloheteroalkyl, halocycloalkyl, halocycloheteroalkyl, substituted or unsubstituted heteroaryl, substituted or unsubstituted -alkylene-aryl, substituted or unsubstituted -alkylene-heteroaryl or Rₐ and R_{b} taken together with the nitrogen atom to which they are attached may form a 3-8 membered ring;
R₁ is 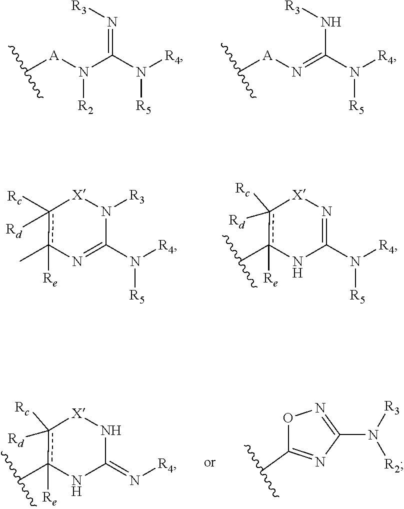
R₂, R₃, R₄, and R₅ are independently selected from H, alkyl, alkenyl, alkynyl, heteroalkyl, cycloalkyl, haloalkyl, halocycloalkyl, cycloheteroalkyl, substituted or unsubstituted aryl, substituted or unsubstituted -alkylene-aryl, substituted or unsubstituted heteroaryl, substituted or unsubstituted -alkylene-heteroaryl, substituted or unsubstituted 5-12 membered ring, alkylenealkoxy, haloalkyl, —CN, —C(O)Rₐ, and —C(O)NRₐR_{b}; provided that (i) no more than one of R₂, R₃, R₄, and R₅ is —CN, (ii) no more than one of R₂, R₃, R₄, and R₅ is —C(O)Rₐ, and (iii) no more than one of R₂, R₃, R₄, and R₅ is —C(O)NRₐR_{b};
R₃ and R₄ taken together may form a 5-8 membered ring;
R₂ and R₅ taken together may form a 5-8 membered ring;
R₄ and R₅ taken together may form a 5-8 membered ring;
R_{c} is H, or alkyl;
R_{d} and Rₑ are independently absent, H, or alkyl;
Rₓ and R_{y} are independently H, F, alkyl, aryl, or haloalkyl; [symbol]
represents a single bond or a double bond; if [symbol] is a double bond, then R_{d} and Rₑ are absent;
A is absent, —CH₂—, —C(O)—, —C(S)—, —S(O)₂—, or —CR_{f}R_{g}—;
X′ is absent, —CH₂—, —C(O)—, —C(S)—, or —S(O)₂—; and
R_{f} and R_{g} are independently selected from H, alkyl, alkenyl, alkynyl, substituted or unsubstituted aryl, substituted or unsubstituted heteroaryl, substituted or unsubstituted -alkylene-aryl, substituted or unsubstituted -alkylene-heteroaryl, and halide; or R_{f} and R_{g} taken together may form a spirocyclic 3-8 membered ring, or heterospirocyclic 3-8 membered ring.

2. The compound of claim 1, wherein R₁ is

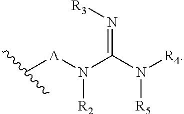

3. The compound of claim 1, wherein R₁ is

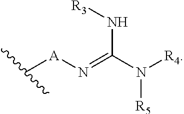

4. The compound of claim 1, wherein R₁

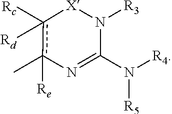

5. The compound of claim 1, wherein R₁ is 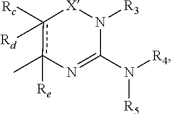
and R₃ is H.

6. The compound of claim 1, wherein R₁ is

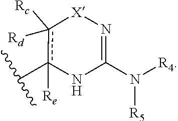

7. The compound of claim 1, wherein R₁ is

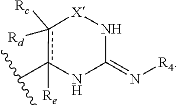

8. The compound of claim 1, wherein R₁ is

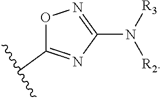

9. A compound of Formula (III): 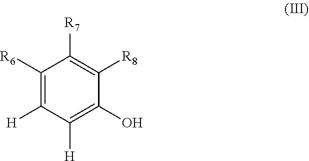
wherein:
R₆ is selected from H, alkyl, cycloalkyl, heteroalkyl, cycloheteroalkyl, substituted or unsubstituted aryl, substituted or unsubstituted -alkylene-aryl, substituted or unsubstituted heteroaryl, substituted or unsubstituted -alkylene-heteroaryl, haloalkyl, halocycloalkyl, halocycloheteroalkyl, —O-alkyl, —O-haloalkyl, —O-cycloalkyl, —N-alkyl, —N-haloalkyl, —S-alkyl, —O-heteroalkyl, —N-heteroalkyl, —S-heteroalkyl, —O-aryl, —N-aryl, —S-aryl, —S-haloalkyl, —S-cycloalkyl, —O-heteroaryl, —O-cycloheteroalkyl, —N-heteroaryl, —N-cycloalkyl, —N-cycloheteroalkyl, —S-cycloheteroalkyl, —S-heteroaryl, halide, —CN, —NO₂, —S(O)Rₐ, —S(O)₂Rₐ, —C(O)Rₐ, —C(O)₂Rₐ, and —C(O)NRₐR_{b};
Rₐ and R_{b} are independently H, alkyl, alkenyl, alkynyl substituted or unsubstituted aryl, cycloalkyl, heteroalkyl, haloalkyl, cycloheteroalkyl, halocycloalkyl, halocycloheteroalkyl, substituted or unsubstituted heteroaryl, substituted or unsubstituted -alkylene-aryl, substituted or unsubstituted -alkylene-heteroaryl or Rₐ and R_{b} taken together with the nitrogen atom to which they are attached may form a 3-8 membered ring;
R₇ is H, halide, alkyl, or aryl;
R₈ is 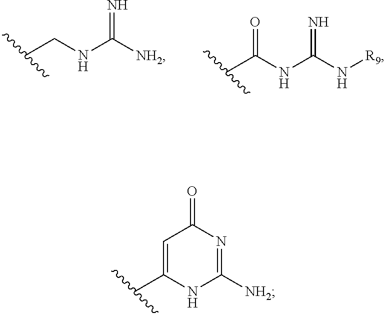
and
R₉ is selected from H, alkyl, alkenyl, alkynyl, heteroalkyl, cycloalkyl, haloalkyl, halocycloalkyl, cycloheteroalkyl, substituted or unsubstituted aryl, substituted or unsubstituted -alkylene-aryl, substituted or unsubstituted heteroaryl, substituted or unsubstituted -alkylene-heteroaryl, —CN, —C(O)Rₐ, and —C(O)NRₐR_{b}.

10. The compound of claim 9, wherein R₆ is selected from halide, —CN, —CF₃, —OCF₃, —SO₂Me, and —NO₂.

11. The compound of claim 10, wherein R₆ is halide.

12. The compound of claim 11, wherein R₆ is Cl, F, or Br.

13. The compound of claim 10, wherein R₆ is —CN.

14. The compound of claim 10, wherein R₆ is —CF₃.

15. The compound of claim 10, wherein R₆ is —OCF₃.

16. The compound of claim 10, wherein R₆ is —SO₂Me.

17. The compound of claim 10, wherein R₆ is —NO₂.

18. The compound of any one of claims 9-17, wherein R₇ is H.

19. The compound of any one of claims 9-17, wherein R₇ is halide.

20. The compound of claim 19, wherein R₇ is Cl or F.

21. The compound of any one of claims 9-17, wherein R₇ is alkyl.

22. The compound of claim 21, wherein R₇ is methyl, ethyl, n-propyl, iso-propyl, n-butyl, iso-butyl, sec-butyl, or t-butyl.

23. The compound of claim 22, wherein R₇ is methyl.

24. The compound of any one of claims 9-17, wherein R₇ is aryl.

25. The compound of claim 24, wherein R₇ is phenyl.

26. The compound of any one of claims 9-25, wherein R₈ is

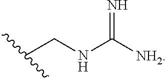 [SMILES: *CNC(=N)N]

27. The compound of any one of claims 9-25, wherein R₈ is

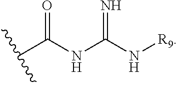

28. The compound of any one of claims 9-25, wherein R₈ is

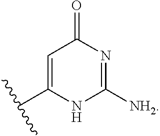 [SMILES: *c1cc(=O)nc(N)[nH]1]

29. The compound of any one of claims 9-28, wherein R₉ is H.

30. The compound of any one of claims 9-28, wherein R₉ is alkyl.

31. The compound of any one of claims 9-28, wherein R₉ is unsubstituted heteroaryl.

32. The compound of any one of claims 9-28, wherein R₉ is alkylene-aryl.

33. The compound of any one of claims 9-28, wherein R₉ is alkylene-heteroaryl.

34. The compound of any one of claims 9-28, wherein R₉ is alkylene-CF₃.

35. The compound of any one of claims 9-28, wherein R₉ is alkylene-OMe.

36. The compound of any one of claims 9-28, wherein R₉ is 5-12 membered ring.

37. The compound of claim 9, wherein the compound is selected from:

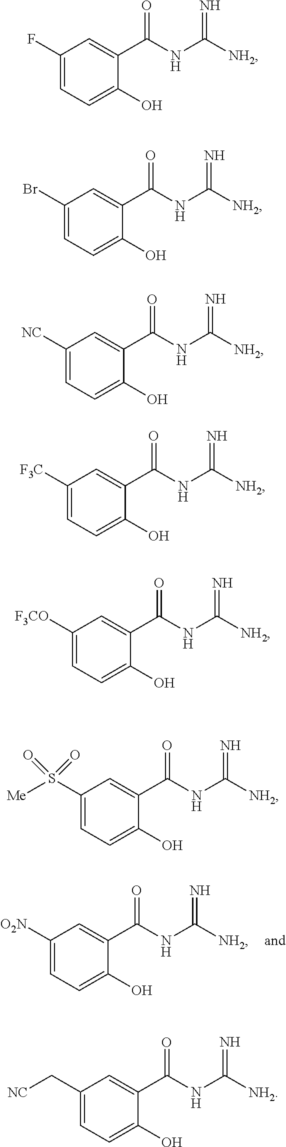

38. The compound of claim 9, wherein the compound is

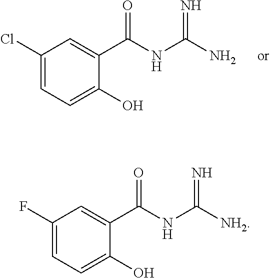 [SMILES: N=C(N)NC(=O)c1cc(Cl)ccc1O ; N=C(N)NC(=O)c1cc(F)ccc1O]

39. The compound of claim 9, wherein the compound is selected from:

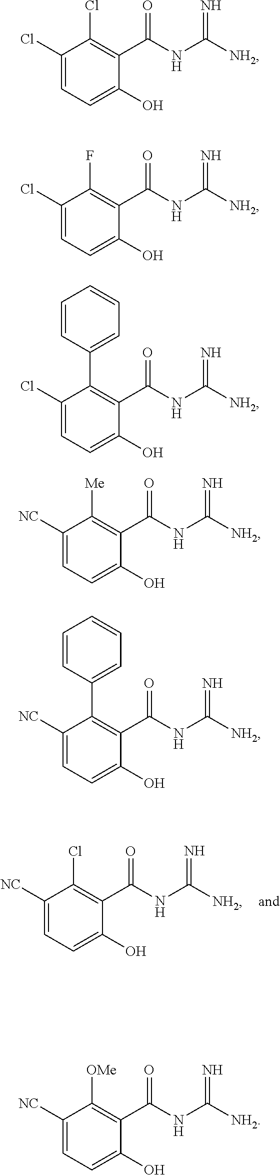

40. The compound of claim 1, wherein the compound is selected from:

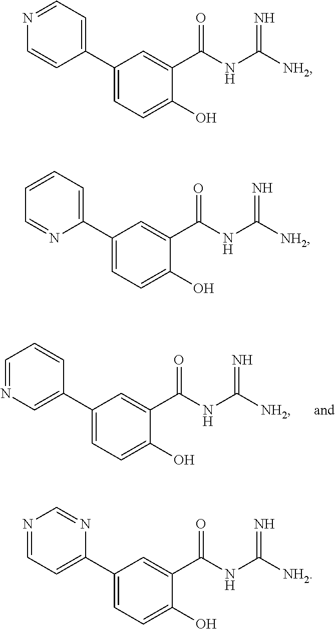 [SMILES: N=C(N)NC(=O)c1cc(-c2ccncc2)ccc1O ; N=C(N)NC(=O)c1cc(-c2ccccn2)ccc1O ; N=C(N)NC(=O)c1cc(-c2cccnc2)ccc1O ; N=C(N)NC(=O)c1cc(-c2ccncn2)ccc1O]

41. The compound of claim 1, wherein the compound is

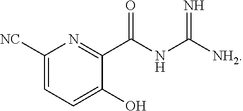 [SMILES: N#Cc1ccc(O)c(C(=O)NC(=N)N)n1]

42. The compound of claim 1, wherein the compound is

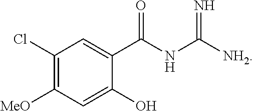 [SMILES: COc1cc(O)c(C(=O)NC(=N)N)cc1Cl]

43. The compound of claim 1, wherein the compound is

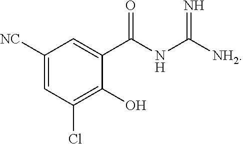 [SMILES: N#Cc1cc(Cl)c(O)c(C(=O)NC(=N)N)c1]

44. The compound of claim 1, wherein the compound is selected from

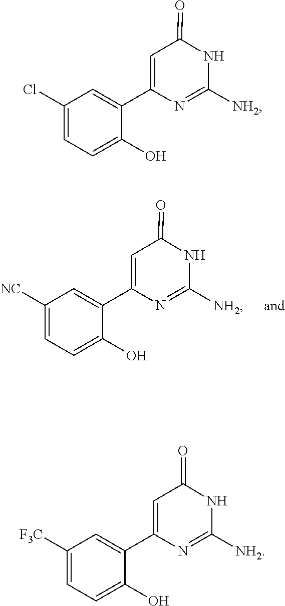 [SMILES: Nc1nc(-c2cc(Cl)ccc2O)cc(=O)[nH]1 ; N#Cc1ccc(O)c(-c2cc(=O)[nH]c(N)n2)c1 ; Nc1nc(-c2cc(C(F)(F)F)ccc2O)cc(=O)[nH]1]

45. The compound of claim 1, wherein the compound is selected from

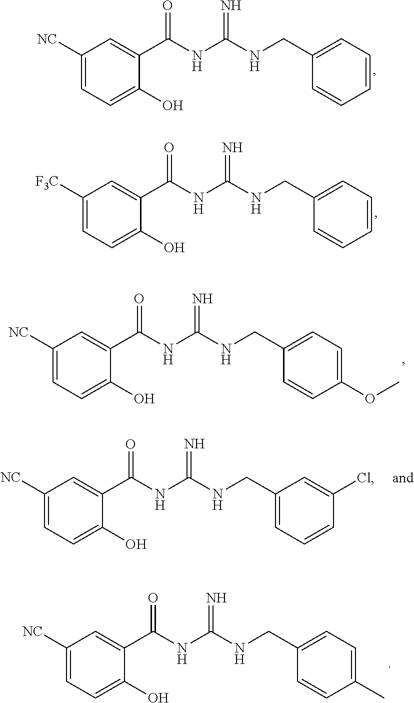

46. The compound of claim 1, wherein the compound is selected from

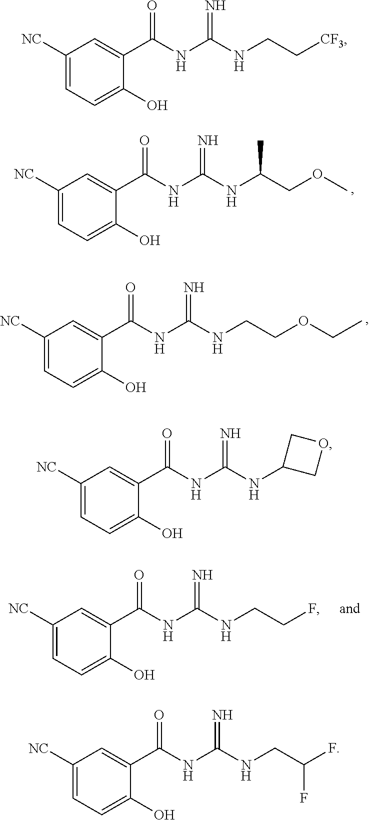

47. The compound of claim 1, wherein the compound is

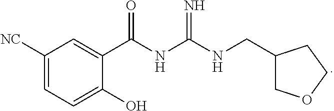 [SMILES: N#Cc1ccc(O)c(C(=O)NC(=N)NCC2CCOC2)c1]

48. The compound of claim 1, wherein the compound is selected from

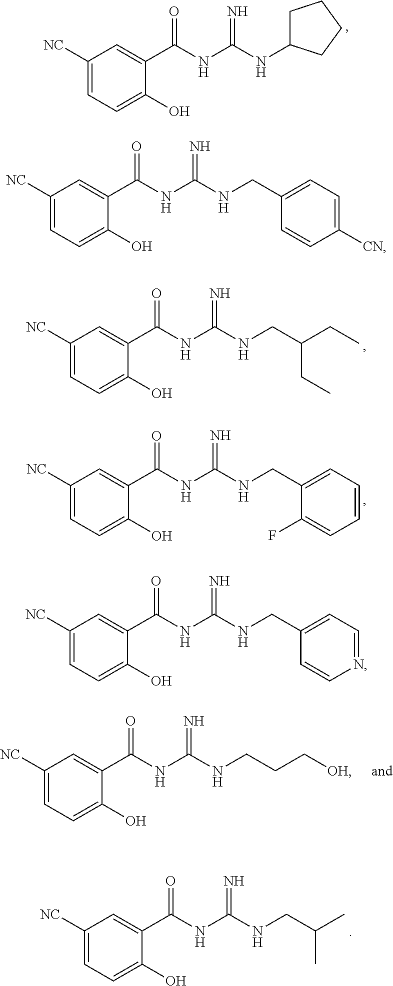

49. The compound of claim 1, wherein the compound is selected from

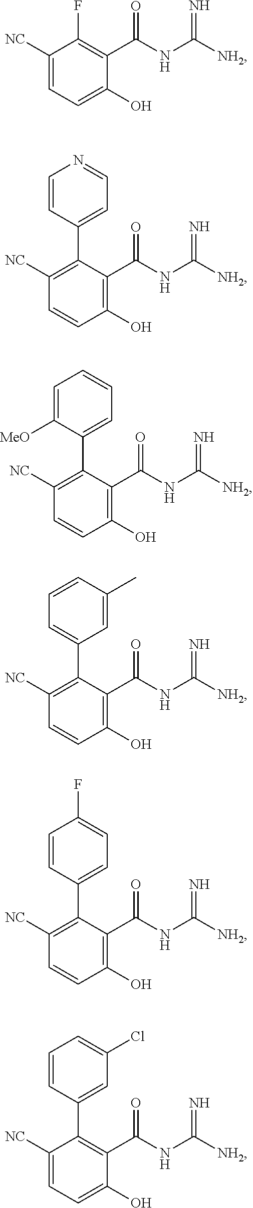

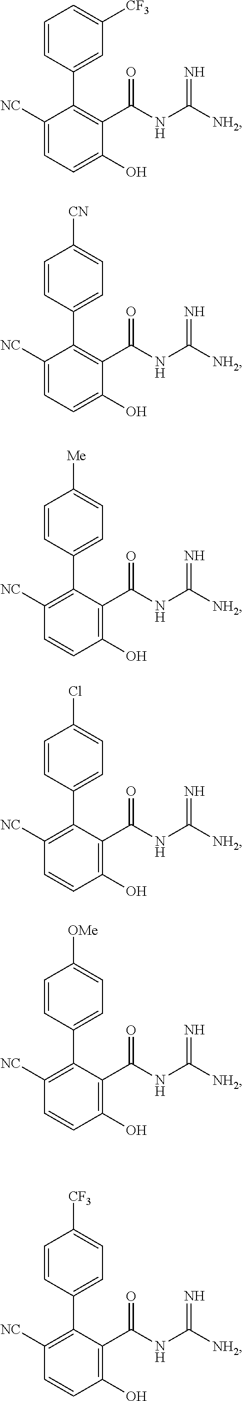

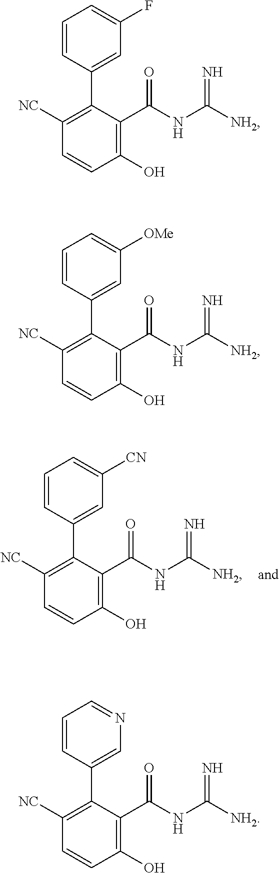 [SMILES: N#Cc1ccc(O)c(C(=O)NC(=N)N)c1-c1cccc(F)c1 ; COc1cccc(-c2c(C#N)ccc(O)c2C(=O)NC(=N)N)c1 ; N#Cc1cccc(-c2c(C#N)ccc(O)c2C(=O)NC(=N)N)c1 ; "and" ; N#Cc1ccc(O)c(C(=O)NC(=N)N)c1-c1cccnc1]

50. The compound of claim 1, wherein the compound is

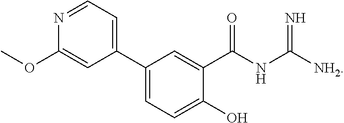 [SMILES: COc1cc(-c2ccc(O)c(C(=O)NC(=N)N)c2)ccn1]

51. The compound of claim 1, wherein the compound is selected from the following table:

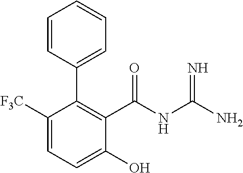 [SMILES: N=C(N)NC(=O)c1c(O)ccc(C(F)(F)F)c1-c1ccccc1]

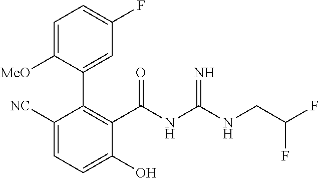 [SMILES: COc1ccc(F)cc1-c1c(C#N)ccc(O)c1C(=O)NC(=N)NCC(F)F]

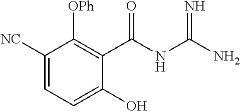 [SMILES: N#Cc1ccc(O)c(C(=O)NC(=N)N)c1Oc1ccccc1]

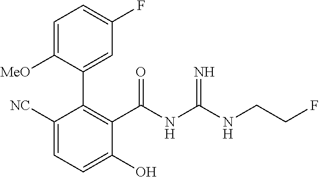 [SMILES: COc1ccc(F)cc1-c1c(C#N)ccc(O)c1C(=O)NC(=N)NCCF]

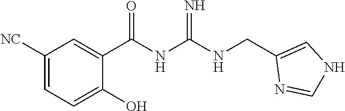 [SMILES: N#Cc1ccc(O)c(C(=O)NC(=N)NCc2c[nH]cn2)c1]

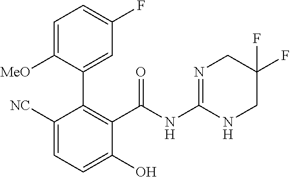 [SMILES: COc1ccc(F)cc1-c1c(C#N)ccc(O)c1C(=O)NC1=NCC(F)(F)CN1]

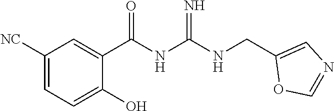 [SMILES: N#Cc1ccc(O)c(C(=O)NC(=N)NCc2cnco2)c1]

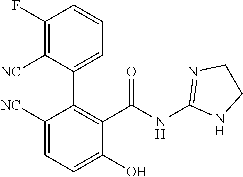 [SMILES: N#Cc1ccc(O)c(C(=O)NC2=NCCN2)c1-c1cccc(F)c1C#N]

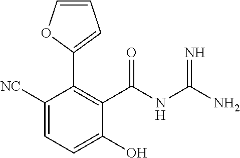 [SMILES: N#Cc1ccc(O)c(C(=O)NC(=N)N)c1-c1ccco1]

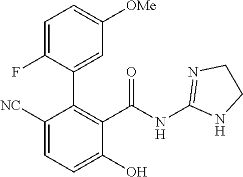 [SMILES: COc1ccc(F)c(-c2c(C#N)ccc(O)c2C(=O)NC2=NCCN2)c1]

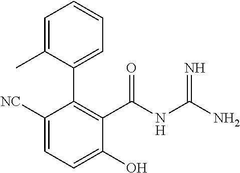 [SMILES: Cc1ccccc1-c1c(C#N)ccc(O)c1C(=O)NC(=N)N]

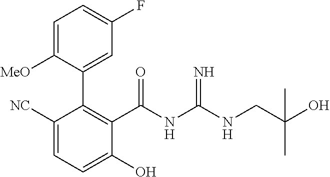 [SMILES: COc1ccc(F)cc1-c1c(C#N)ccc(O)c1C(=O)NC(=N)NCC(C)(C)O]

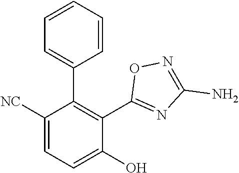 [SMILES: N#Cc1ccc(O)c(-c2nc(N)no2)c1-c1ccccc1]

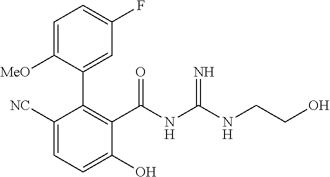 [SMILES: COc1ccc(F)cc1-c1c(C#N)ccc(O)c1C(=O)NC(=N)NCCO]

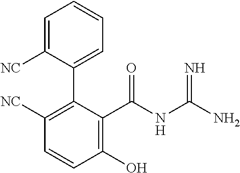 [SMILES: N#Cc1ccccc1-c1c(C#N)ccc(O)c1C(=O)NC(=N)N]

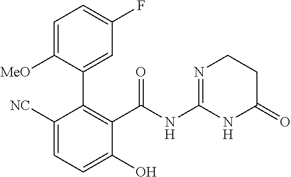 [SMILES: COc1ccc(F)cc1-c1c(C#N)ccc(O)c1C(=O)NC1=NCCC(=O)N1]

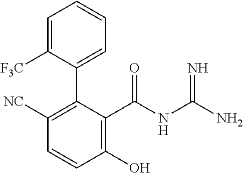 [SMILES: N#Cc1ccc(O)c(C(=O)NC(=N)N)c1-c1ccccc1C(F)(F)F]

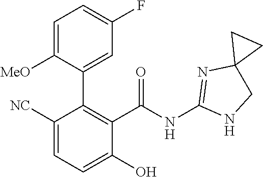 [SMILES: COc1ccc(F)cc1-c1c(C#N)ccc(O)c1C(=O)NC1=NC2(CC2)CN1]

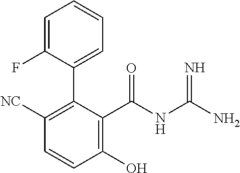 [SMILES: N#Cc1ccc(O)c(C(=O)NC(=N)N)c1-c1ccccc1F]

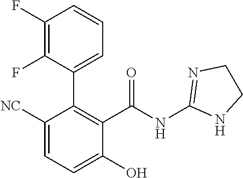 [SMILES: N#Cc1ccc(O)c(C(=O)NC2=NCCN2)c1-c1cccc(F)c1F]

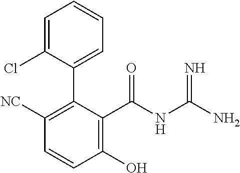 [SMILES: N#Cc1ccc(O)c(C(=O)NC(=N)N)c1-c1ccccc1Cl]

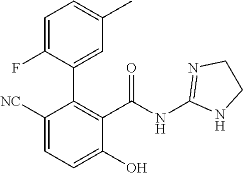 [SMILES: Cc1ccc(F)c(-c2c(C#N)ccc(O)c2C(=O)NC2=NCCN2)c1]

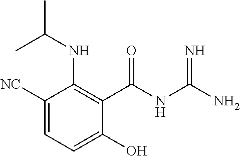 [SMILES: CC(C)Nc1c(C#N)ccc(O)c1C(=O)NC(=N)N]

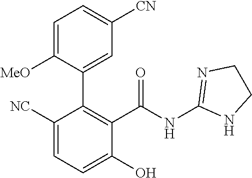 [SMILES: COc1ccc(C#N)cc1-c1c(C#N)ccc(O)c1C(=O)NC1=NCCN1]

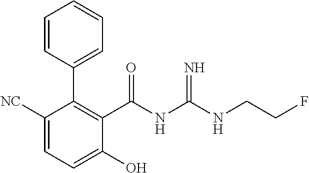 [SMILES: N#Cc1ccc(O)c(C(=O)NC(=N)NCCF)c1-c1ccccc1]

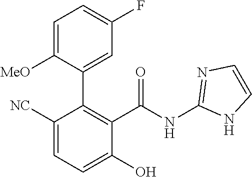 [SMILES: COc1ccc(F)cc1-c1c(C#N)ccc(O)c1C(=O)Nc1ncc[nH]1]

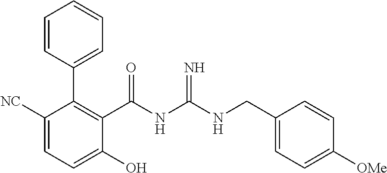 [SMILES: COc1ccc(CNC(=N)NC(=O)c2c(O)ccc(C#N)c2-c2ccccc2)cc1]

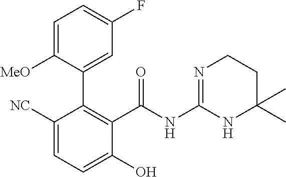 [SMILES: COc1ccc(F)cc1-c1c(C#N)ccc(O)c1C(=O)NC1=NCCC(C)(C)N1]

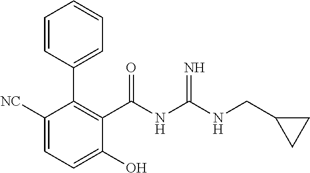 [SMILES: N#Cc1ccc(O)c(C(=O)NC(=N)NCC2CC2)c1-c1ccccc1]

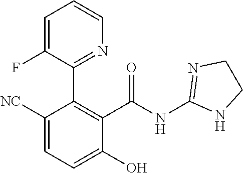 [SMILES: N#Cc1ccc(O)c(C(=O)NC2=NCCN2)c1-c1ncccc1F]

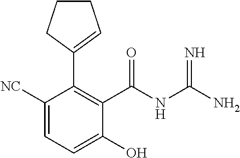 [SMILES: N#Cc1ccc(O)c(C(=O)NC(=N)N)c1C1=CCCC1]

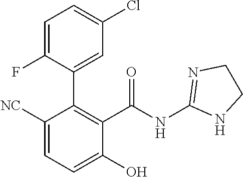 [SMILES: N#Cc1ccc(O)c(C(=O)NC2=NCCN2)c1-c1cc(Cl)ccc1F]

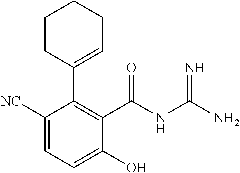 [SMILES: N#Cc1ccc(O)c(C(=O)NC(=N)N)c1C1=CCCCC1]

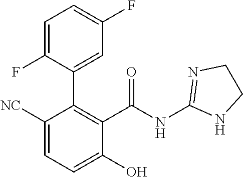 [SMILES: N#Cc1ccc(O)c(C(=O)NC2=NCCN2)c1-c1cc(F)ccc1F]

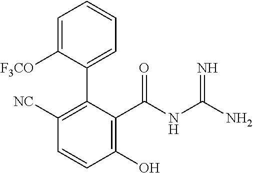 [SMILES: N#Cc1ccc(O)c(C(=O)NC(=N)N)c1-c1ccccc1OC(F)(F)F]

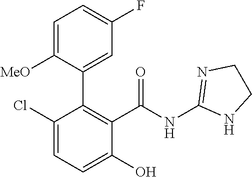 [SMILES: COc1ccc(F)cc1-c1c(Cl)ccc(O)c1C(=O)NC1=NCCN1]

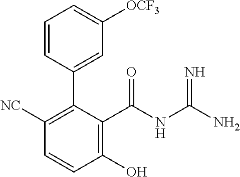 [SMILES: N#Cc1ccc(O)c(C(=O)NC(=N)N)c1-c1cccc(OC(F)(F)F)c1]

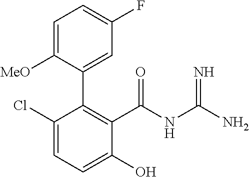 [SMILES: COc1ccc(F)cc1-c1c(Cl)ccc(O)c1C(=O)NC(=N)N]

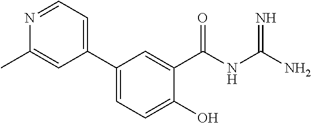 [SMILES: Cc1cc(-c2ccc(O)c(C(=O)NC(=N)N)c2)ccn1]

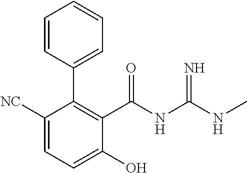 [SMILES: CNC(=N)NC(=O)c1c(O)ccc(C#N)c1-c1ccccc1]

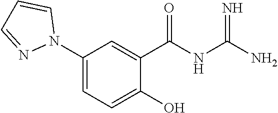 [SMILES: N=C(N)NC(=O)c1cc(-n2cccn2)ccc1O]

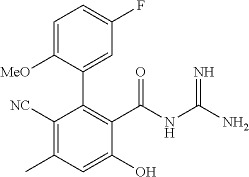 [SMILES: COc1ccc(F)cc1-c1c(C#N)c(C)cc(O)c1C(=O)NC(=N)N]

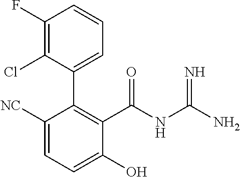 [SMILES: N#Cc1ccc(O)c(C(=O)NC(=N)N)c1-c1cccc(F)c1Cl]

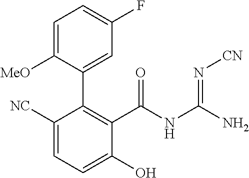 [SMILES: COc1ccc(F)cc1-c1c(C#N)ccc(O)c1C(=O)N/C(N)=N/C#N]

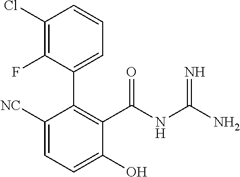 [SMILES: N#Cc1ccc(O)c(C(=O)NC(=N)N)c1-c1cccc(Cl)c1F]

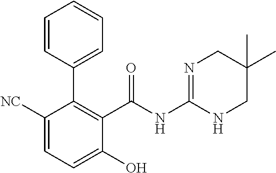 [SMILES: CC1(C)CN=C(NC(=O)c2c(O)ccc(C#N)c2-c2ccccc2)NC1]

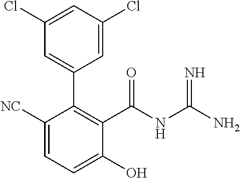 [SMILES: N#Cc1ccc(O)c(C(=O)NC(=N)N)c1-c1cc(Cl)cc(Cl)c1]

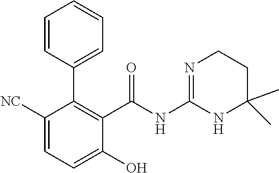 [SMILES: CC1(C)CCN=C(NC(=O)c2c(O)ccc(C#N)c2-c2ccccc2)N1]

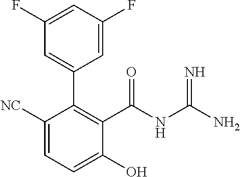 [SMILES: N#Cc1ccc(O)c(C(=O)NC(=N)N)c1-c1cc(F)cc(F)c1]

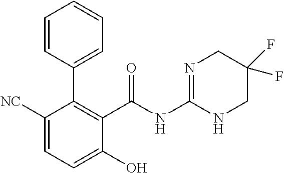 [SMILES: N#Cc1ccc(O)c(C(=O)NC2=NCC(F)(F)CN2)c1-c1ccccc1]

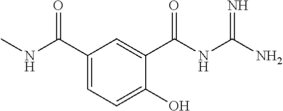 [SMILES: CNC(=O)c1ccc(O)c(C(=O)NC(=N)N)c1]

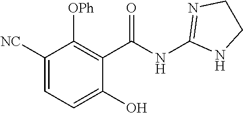 [SMILES: N#Cc1ccc(O)c(C(=O)NC2=NCCN2)c1Oc1ccccc1]

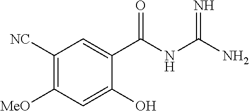 [SMILES: COc1cc(O)c(C(=O)NC(=N)N)cc1C#N]

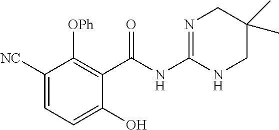 [SMILES: CC1(C)CN=C(NC(=O)c2c(O)ccc(C#N)c2Oc2ccccc2)NC1]

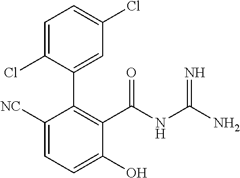 [SMILES: N#Cc1ccc(O)c(C(=O)NC(=N)N)c1-c1cc(Cl)ccc1Cl]

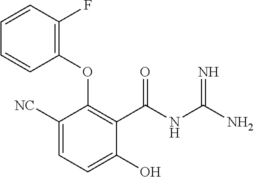 [SMILES: N#Cc1ccc(O)c(C(=O)NC(=N)N)c1Oc1ccccc1F]

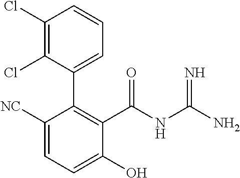 [SMILES: N#Cc1ccc(O)c(C(=O)NC(=N)N)c1-c1cccc(Cl)c1Cl]

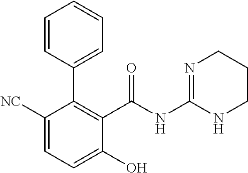 [SMILES: N#Cc1ccc(O)c(C(=O)NC2=NCCCN2)c1-c1ccccc1]

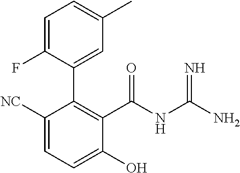 [SMILES: Cc1ccc(F)c(-c2c(C#N)ccc(O)c2C(=O)NC(=N)N)c1]

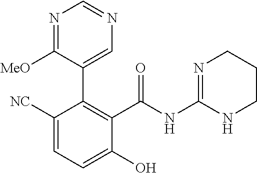 [SMILES: COc1ncncc1-c1c(C#N)ccc(O)c1C(=O)NC1=NCCCN1]

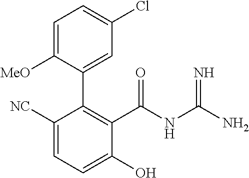 [SMILES: COc1ccc(Cl)cc1-c1c(C#N)ccc(O)c1C(=O)NC(=N)N]

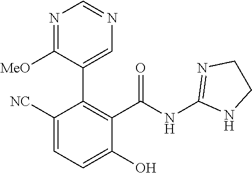 [SMILES: COc1ncncc1-c1c(C#N)ccc(O)c1C(=O)NC1=NCCN1]

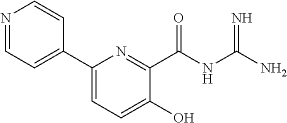 [SMILES: N=C(N)NC(=O)c1nc(-c2ccncc2)ccc1O]

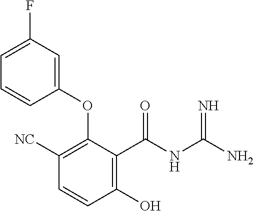 [SMILES: N#Cc1ccc(O)c(C(=O)NC(=N)N)c1Oc1cccc(F)c1]

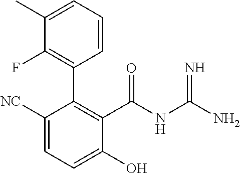 [SMILES: Cc1cccc(-c2c(C#N)ccc(O)c2C(=O)NC(=N)N)c1F]

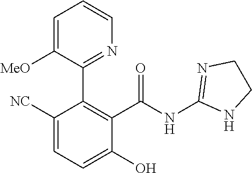 [SMILES: COc1cccnc1-c1c(C#N)ccc(O)c1C(=O)NC1=NCCN1]

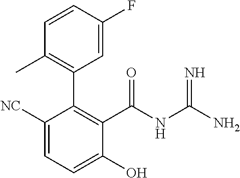 [SMILES: Cc1ccc(F)cc1-c1c(C#N)ccc(O)c1C(=O)NC(=N)N]

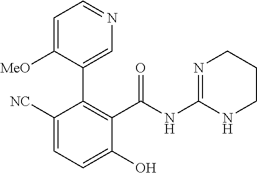 [SMILES: COc1ccncc1-c1c(C#N)ccc(O)c1C(=O)NC1=NCCCN1]

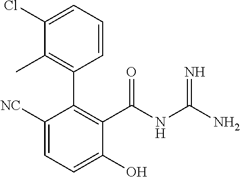 [SMILES: Cc1c(Cl)cccc1-c1c(C#N)ccc(O)c1C(=O)NC(=N)N]

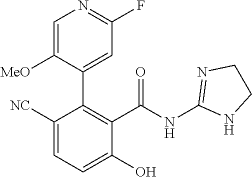 [SMILES: COc1cnc(F)cc1-c1c(C#N)ccc(O)c1C(=O)NC1=NCCN1]

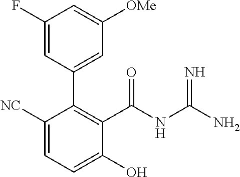 [SMILES: COc1cc(F)cc(-c2c(C#N)ccc(O)c2C(=O)NC(=N)N)c1]

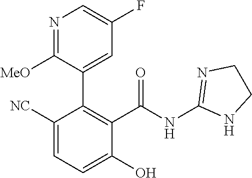 [SMILES: COc1ncc(F)cc1-c1c(C#N)ccc(O)c1C(=O)NC1=NCCN1]

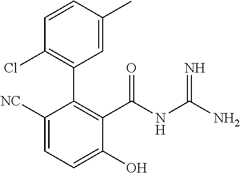 [SMILES: Cc1ccc(Cl)c(-c2c(C#N)ccc(O)c2C(=O)NC(=N)N)c1]

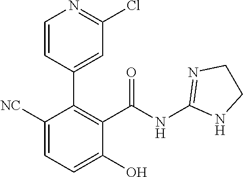 [SMILES: N#Cc1ccc(O)c(C(=O)NC2=NCCN2)c1-c1ccnc(Cl)c1]

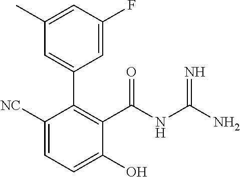 [SMILES: Cc1cc(F)cc(-c2c(C#N)ccc(O)c2C(=O)NC(=N)N)c1]

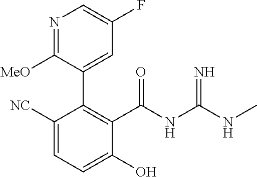 [SMILES: CNC(=N)NC(=O)c1c(O)ccc(C#N)c1-c1cc(F)cnc1OC]

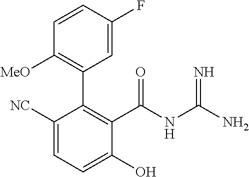 [SMILES: COc1ccc(F)cc1-c1c(C#N)ccc(O)c1C(=O)NC(=N)N]

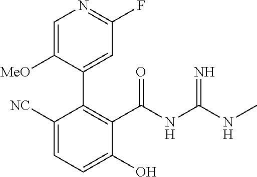 [SMILES: CNC(=N)NC(=O)c1c(O)ccc(C#N)c1-c1cc(F)ncc1OC]

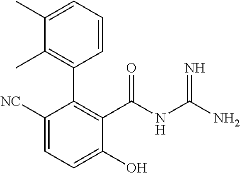 [SMILES: Cc1cccc(-c2c(C#N)ccc(O)c2C(=O)NC(=N)N)c1C]

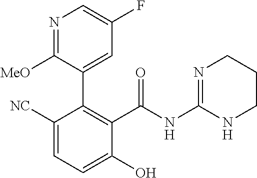 [SMILES: COc1ncc(F)cc1-c1c(C#N)ccc(O)c1C(=O)NC1=NCCCN1]

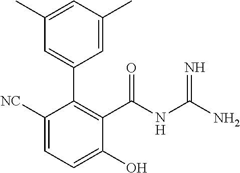 [SMILES: Cc1cc(C)cc(-c2c(C#N)ccc(O)c2C(=O)NC(=N)N)c1]

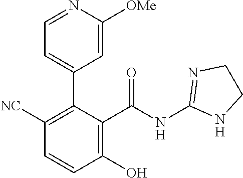 [SMILES: COc1cc(-c2c(C#N)ccc(O)c2C(=O)NC2=NCCN2)ccn1]

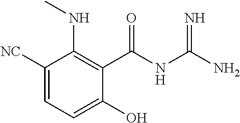 [SMILES: CNc1c(C#N)ccc(O)c1C(=O)NC(=N)N]

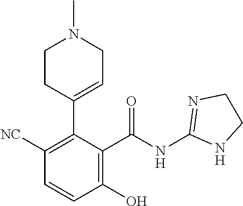 [SMILES: CN1CC=C(c2c(C#N)ccc(O)c2C(=O)NC2=NCCN2)CC1]

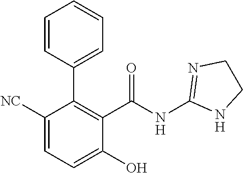 [SMILES: N#Cc1ccc(O)c(C(=O)NC2=NCCN2)c1-c1ccccc1]

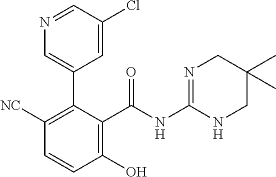 [SMILES: CC1(C)CN=C(NC(=O)c2c(O)ccc(C#N)c2-c2cncc(Cl)c2)NC1]

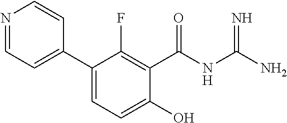 [SMILES: N=C(N)NC(=O)c1c(O)ccc(-c2ccncc2)c1F]

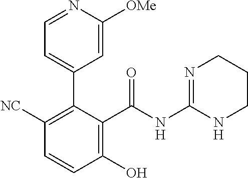 [SMILES: COc1cc(-c2c(C#N)ccc(O)c2C(=O)NC2=NCCCN2)ccn1]

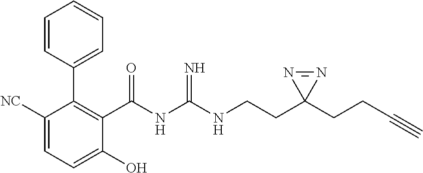 [SMILES: C#CCCC1(CCNC(=N)NC(=O)c2c(O)ccc(C#N)c2-c2ccccc2)N=N1]

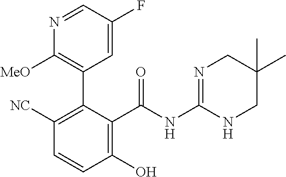 [SMILES: COc1ncc(F)cc1-c1c(C#N)ccc(O)c1C(=O)NC1=NCC(C)(C)CN1]

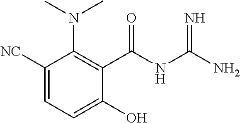 [SMILES: CN(C)c1c(C#N)ccc(O)c1C(=O)NC(=N)N]

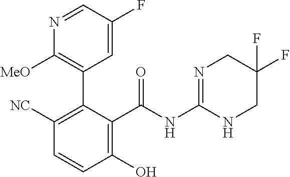 [SMILES: COc1ncc(F)cc1-c1c(C#N)ccc(O)c1C(=O)NC1=NCC(F)(F)CN1]

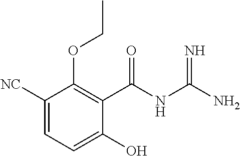 [SMILES: CCOc1c(C#N)ccc(O)c1C(=O)NC(=N)N]

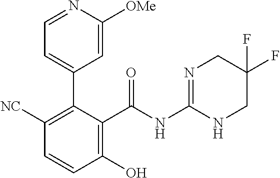 [SMILES: COc1cc(-c2c(C#N)ccc(O)c2C(=O)NC2=NCC(F)(F)CN2)ccn1]

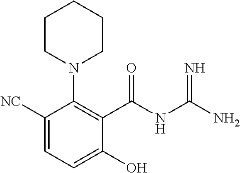 [SMILES: N#Cc1ccc(O)c(C(=O)NC(=N)N)c1N1CCCCC1]

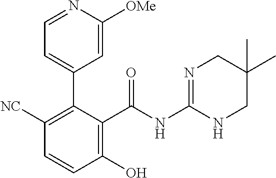 [SMILES: COc1cc(-c2c(C#N)ccc(O)c2C(=O)NC2=NCC(C)(C)CN2)ccn1]

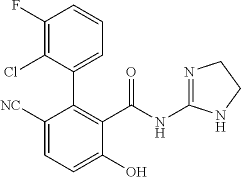 [SMILES: N#Cc1ccc(O)c(C(=O)NC2=NCCN2)c1-c1cccc(F)c1Cl]

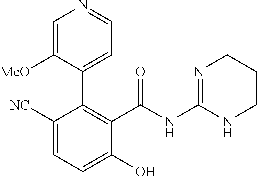 [SMILES: COc1cnccc1-c1c(C#N)ccc(O)c1C(=O)NC1=NCCCN1]

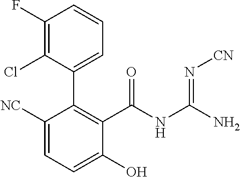 [SMILES: N#C/N=C(\N)NC(=O)c1c(O)ccc(C#N)c1-c1cccc(F)c1Cl]

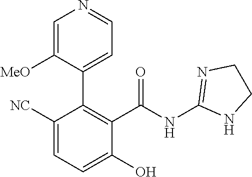 [SMILES: COc1cnccc1-c1c(C#N)ccc(O)c1C(=O)NC1=NCCN1]

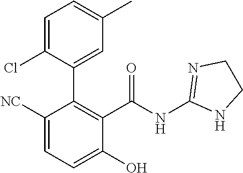 [SMILES: Cc1ccc(Cl)c(-c2c(C#N)ccc(O)c2C(=O)NC2=NCCN2)c1]

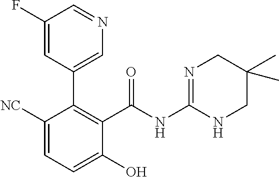 [SMILES: CC1(C)CN=C(NC(=O)c2c(O)ccc(C#N)c2-c2cncc(F)c2)NC1]

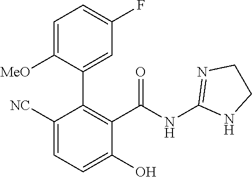 [SMILES: COc1ccc(F)cc1-c1c(C#N)ccc(O)c1C(=O)NC1=NCCN1]

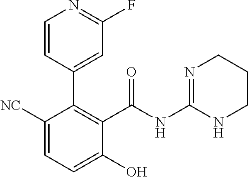 [SMILES: N#Cc1ccc(O)c(C(=O)NC2=NCCCN2)c1-c1ccnc(F)c1]

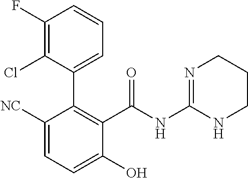 [SMILES: N#Cc1ccc(O)c(C(=O)NC2=NCCCN2)c1-c1cccc(F)c1Cl]

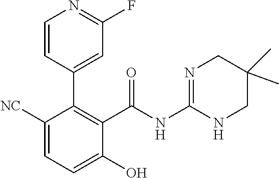 [SMILES: CC1(C)CN=C(NC(=O)c2c(O)ccc(C#N)c2-c2ccnc(F)c2)NC1]

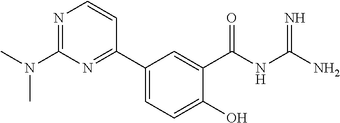 [SMILES: CN(C)c1nccc(-c2ccc(O)c(C(=O)NC(=N)N)c2)n1]

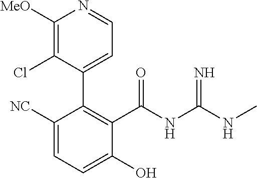 [SMILES: CNC(=N)NC(=O)c1c(O)ccc(C#N)c1-c1ccnc(OC)c1Cl]

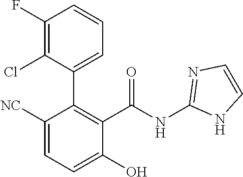 [SMILES: N#Cc1ccc(O)c(C(=O)Nc2ncc[nH]2)c1-c1cccc(F)c1Cl]

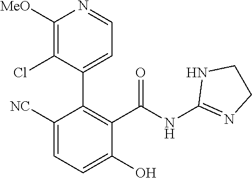 [SMILES: COc1nccc(-c2c(C#N)ccc(O)c2C(=O)NC2=NCCN2)c1Cl]

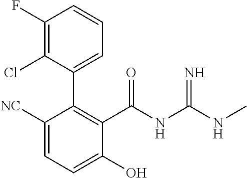 [SMILES: CNC(=N)NC(=O)c1c(O)ccc(C#N)c1-c1cccc(F)c1Cl]

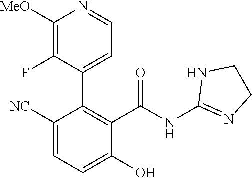 [SMILES: COc1nccc(-c2c(C#N)ccc(O)c2C(=O)NC2=NCCN2)c1F]

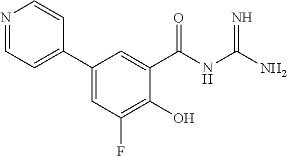 [SMILES: N=C(N)NC(=O)c1cc(-c2ccncc2)cc(F)c1O]

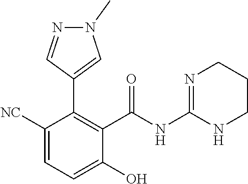 [SMILES: Cn1cc(-c2c(C#N)ccc(O)c2C(=O)NC2=NCCCN2)cn1]

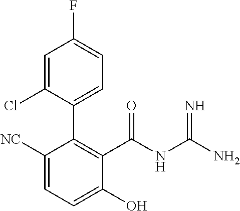 [SMILES: N#Cc1ccc(O)c(C(=O)NC(=N)N)c1-c1ccc(F)cc1Cl]

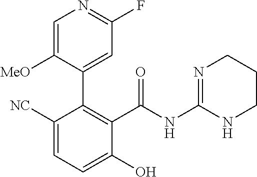 [SMILES: COc1cnc(F)cc1-c1c(C#N)ccc(O)c1C(=O)NC1=NCCCN1]

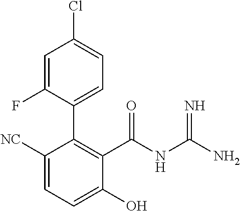 [SMILES: N#Cc1ccc(O)c(C(=O)NC(=N)N)c1-c1ccc(Cl)cc1F]

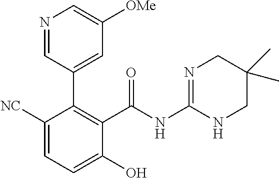 [SMILES: COc1cncc(-c2c(C#N)ccc(O)c2C(=O)NC2=NCC(C)(C)CN2)c1]

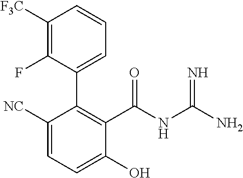 [SMILES: N#Cc1ccc(O)c(C(=O)NC(=N)N)c1-c1cccc(C(F)(F)F)c1F]

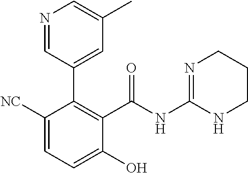 [SMILES: Cc1cncc(-c2c(C#N)ccc(O)c2C(=O)NC2=NCCCN2)c1]

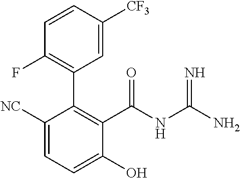 [SMILES: N#Cc1ccc(O)c(C(=O)NC(=N)N)c1-c1cc(C(F)(F)F)ccc1F]

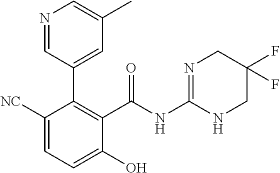 [SMILES: Cc1cncc(-c2c(C#N)ccc(O)c2C(=O)NC2=NCC(F)(F)CN2)c1]

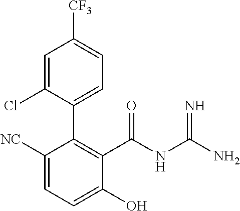 [SMILES: N#Cc1ccc(O)c(C(=O)NC(=N)N)c1-c1ccc(C(F)(F)F)cc1Cl]

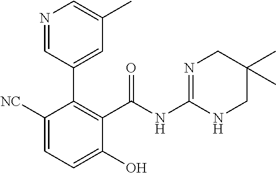 [SMILES: Cc1cncc(-c2c(C#N)ccc(O)c2C(=O)NC2=NCC(C)(C)CN2)c1]

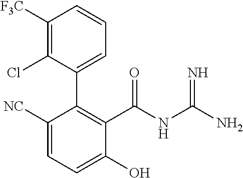 [SMILES: N#Cc1ccc(O)c(C(=O)NC(=N)N)c1-c1cccc(C(F)(F)F)c1Cl]

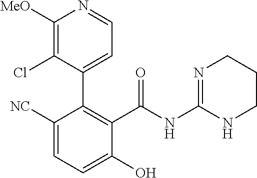 [SMILES: COc1nccc(-c2c(C#N)ccc(O)c2C(=O)NC2=NCCCN2)c1Cl]

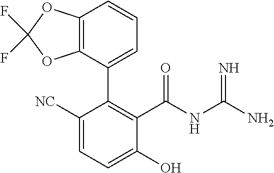 [SMILES: N#Cc1ccc(O)c(C(=O)NC(=N)N)c1-c1cccc2c1OC(F)(F)O2]

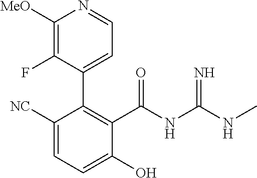 [SMILES: CNC(=N)NC(=O)c1c(O)ccc(C#N)c1-c1ccnc(OC)c1F]

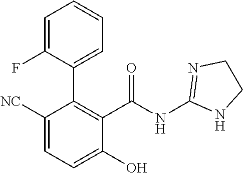 [SMILES: N#Cc1ccc(O)c(C(=O)NC2=NCCN2)c1-c1ccccc1F]

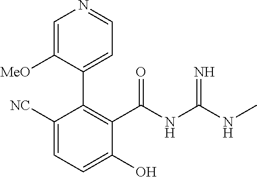 [SMILES: CNC(=N)NC(=O)c1c(O)ccc(C#N)c1-c1ccncc1OC]

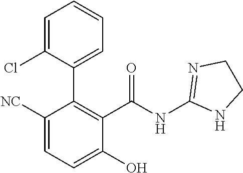 [SMILES: N#Cc1ccc(O)c(C(=O)NC2=NCCN2)c1-c1ccccc1Cl]

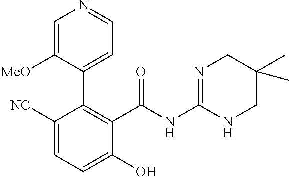 [SMILES: COc1cnccc1-c1c(C#N)ccc(O)c1C(=O)NC1=NCC(C)(C)CN1]

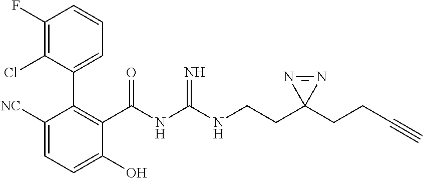 [SMILES: C#CCCC1(CCNC(=N)NC(=O)c2c(O)ccc(C#N)c2-c2cccc(F)c2Cl)N=N1]

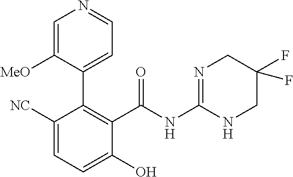 [SMILES: COc1cnccc1-c1c(C#N)ccc(O)c1C(=O)NC1=NCC(F)(F)CN1]

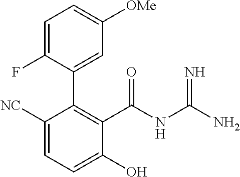 [SMILES: COc1ccc(F)c(-c2c(C#N)ccc(O)c2C(=O)NC(=N)N)c1]

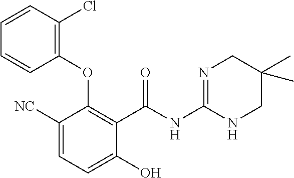 [SMILES: CC1(C)CN=C(NC(=O)c2c(O)ccc(C#N)c2Oc2ccccc2Cl)NC1]

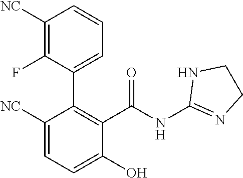 [SMILES: N#Cc1cccc(-c2c(C#N)ccc(O)c2C(=O)NC2=NCCN2)c1F]

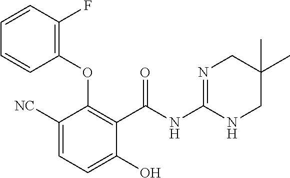 [SMILES: CC1(C)CN=C(NC(=O)c2c(O)ccc(C#N)c2Oc2ccccc2F)NC1]

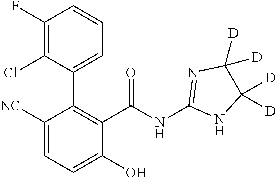 [SMILES: [2H]C1([2H])N=C(NC(=O)c2c(O)ccc(C#N)c2-c2cccc(F)c2Cl)NC1([2H])[2H]]

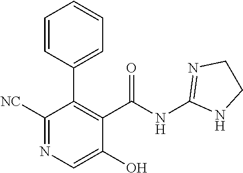 [SMILES: N#Cc1ncc(O)c(C(=O)NC2=NCCN2)c1-c1ccccc1]

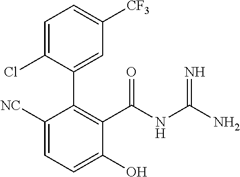 [SMILES: N#Cc1ccc(O)c(C(=O)NC(=N)N)c1-c1cc(C(F)(F)F)ccc1Cl]

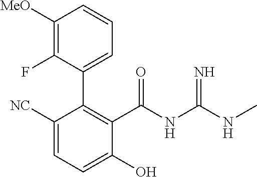 [SMILES: CNC(=N)NC(=O)c1c(O)ccc(C#N)c1-c1cccc(OC)c1F]

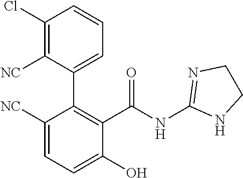 [SMILES: N#Cc1ccc(O)c(C(=O)NC2=NCCN2)c1-c1cccc(Cl)c1C#N]

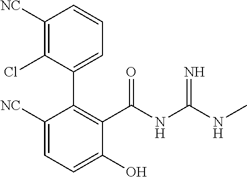 [SMILES: CNC(=N)NC(=O)c1c(O)ccc(C#N)c1-c1cccc(C#N)c1Cl]

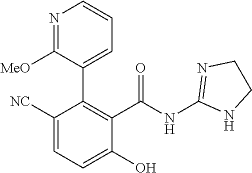 [SMILES: COc1ncccc1-c1c(C#N)ccc(O)c1C(=O)NC1=NCCN1]

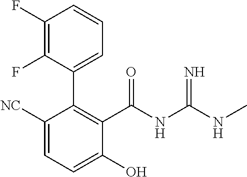 [SMILES: CNC(=N)NC(=O)c1c(O)ccc(C#N)c1-c1cccc(F)c1F]

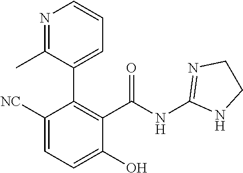 [SMILES: Cc1ncccc1-c1c(C#N)ccc(O)c1C(=O)NC1=NCCN1]

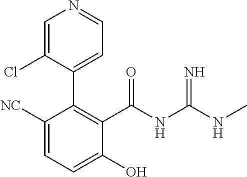 [SMILES: CNC(=N)NC(=O)c1c(O)ccc(C#N)c1-c1ccncc1Cl]

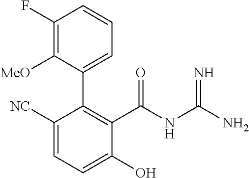 [SMILES: COc1c(F)cccc1-c1c(C#N)ccc(O)c1C(=O)NC(=N)N]

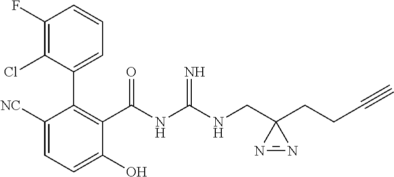 [SMILES: C#CCCC1(CNC(=N)NC(=O)c2c(O)ccc(C#N)c2-c2cccc(F)c2Cl)N=N1]

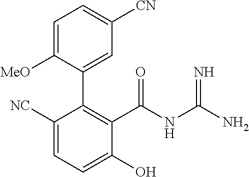 [SMILES: COc1ccc(C#N)cc1-c1c(C#N)ccc(O)c1C(=O)NC(=N)N]

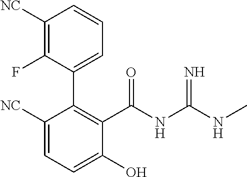 [SMILES: CNC(=N)NC(=O)c1c(O)ccc(C#N)c1-c1cccc(C#N)c1F]

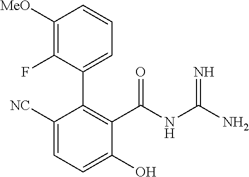 [SMILES: COc1cccc(-c2c(C#N)ccc(O)c2C(=O)NC(=N)N)c1F]

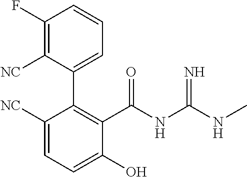 [SMILES: CNC(=N)NC(=O)c1c(O)ccc(C#N)c1-c1cccc(F)c1C#N]

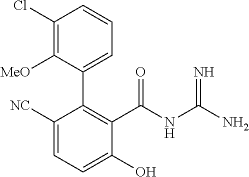 [SMILES: COc1c(Cl)cccc1-c1c(C#N)ccc(O)c1C(=O)NC(=N)N]

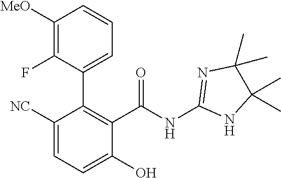 [SMILES: COc1cccc(-c2c(C#N)ccc(O)c2C(=O)NC2=NC(C)(C)C(C)(C)N2)c1F]

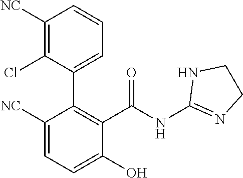 [SMILES: N#Cc1cccc(-c2c(C#N)ccc(O)c2C(=O)NC2=NCCN2)c1Cl]

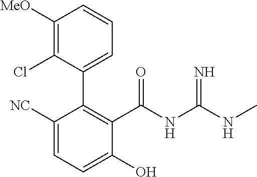 [SMILES: CNC(=N)NC(=O)c1c(O)ccc(C#N)c1-c1cccc(OC)c1Cl]

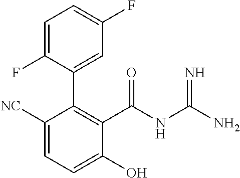 [SMILES: N#Cc1ccc(O)c(C(=O)NC(=N)N)c1-c1cc(F)ccc1F]

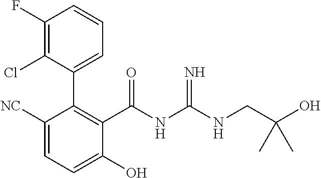 [SMILES: CC(C)(O)CNC(=N)NC(=O)c1c(O)ccc(C#N)c1-c1cccc(F)c1Cl]

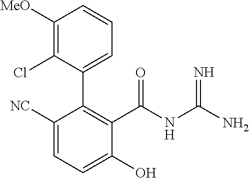 [SMILES: COc1cccc(-c2c(C#N)ccc(O)c2C(=O)NC(=N)N)c1Cl]

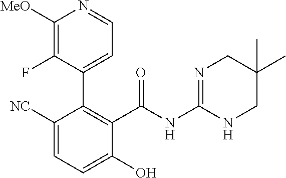 [SMILES: COc1nccc(-c2c(C#N)ccc(O)c2C(=O)NC2=NCC(C)(C)CN2)c1F]

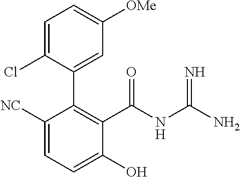 [SMILES: COc1ccc(Cl)c(-c2c(C#N)ccc(O)c2C(=O)NC(=N)N)c1]

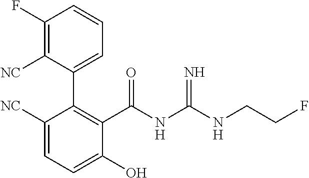 [SMILES: N#Cc1ccc(O)c(C(=O)NC(=N)NCCF)c1-c1cccc(F)c1C#N]

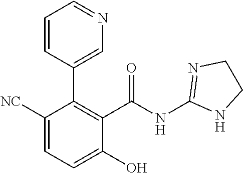 [SMILES: N#Cc1ccc(O)c(C(=O)NC2=NCCN2)c1-c1cccnc1]

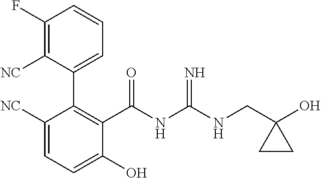 [SMILES: N#Cc1ccc(O)c(C(=O)NC(=N)NCC2(O)CC2)c1-c1cccc(F)c1C#N]

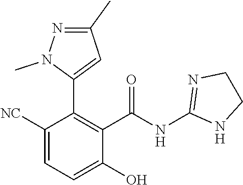 [SMILES: Cc1cc(-c2c(C#N)ccc(O)c2C(=O)NC2=NCCN2)n(C)n1]

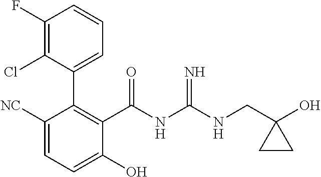 [SMILES: N#Cc1ccc(O)c(C(=O)NC(=N)NCC2(O)CC2)c1-c1cccc(F)c1Cl]

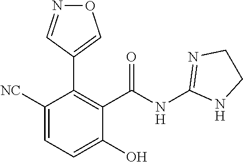 [SMILES: N#Cc1ccc(O)c(C(=O)NC2=NCCN2)c1-c1cnoc1]

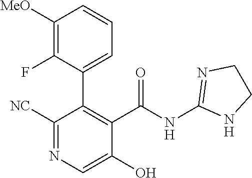 [SMILES: COc1cccc(-c2c(C#N)ncc(O)c2C(=O)NC2=NCCN2)c1F]

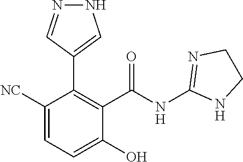 [SMILES: N#Cc1ccc(O)c(C(=O)NC2=NCCN2)c1-c1cn[nH]c1]

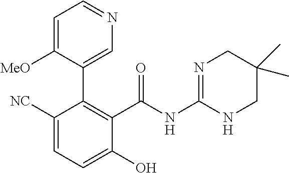 [SMILES: COc1ccncc1-c1c(C#N)ccc(O)c1C(=O)NC1=NCC(C)(C)CN1]

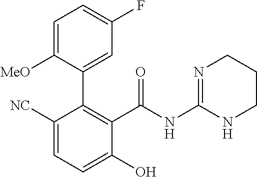 [SMILES: COc1ccc(F)cc1-c1c(C#N)ccc(O)c1C(=O)NC1=NCCCN1]

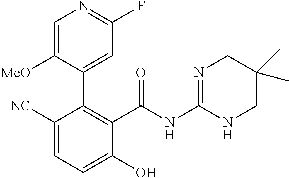 [SMILES: COc1cnc(F)cc1-c1c(C#N)ccc(O)c1C(=O)NC1=NCC(C)(C)CN1]

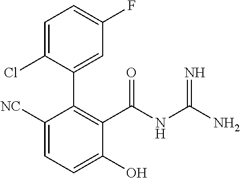 [SMILES: N#Cc1ccc(O)c(C(=O)NC(=N)N)c1-c1cc(F)ccc1Cl]

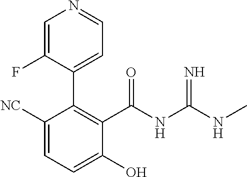 [SMILES: CNC(=N)NC(=O)c1c(O)ccc(C#N)c1-c1ccncc1F]

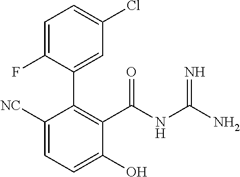 [SMILES: N#Cc1ccc(O)c(C(=O)NC(=N)N)c1-c1cc(Cl)ccc1F]

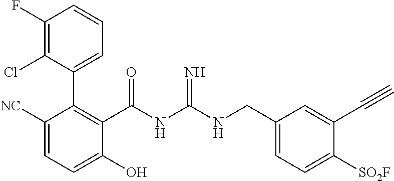 [SMILES: C#Cc1cc(CNC(=N)NC(=O)c2c(O)ccc(C#N)c2-c2cccc(F)c2Cl)ccc1S(=O)(=O)F]

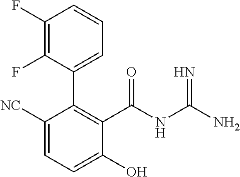 [SMILES: N#Cc1ccc(O)c(C(=O)NC(=N)N)c1-c1cccc(F)c1F]

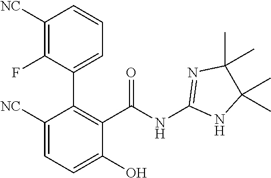 [SMILES: CC1(C)N=C(NC(=O)c2c(O)ccc(C#N)c2-c2cccc(C#N)c2F)NC1(C)C]

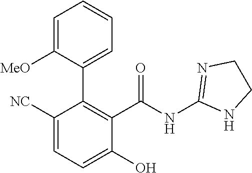 [SMILES: COc1ccccc1-c1c(C#N)ccc(O)c1C(=O)NC1=NCCN1]

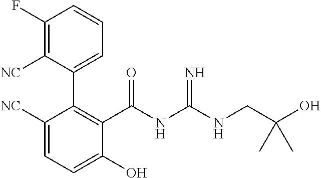 [SMILES: CC(C)(O)CNC(=N)NC(=O)c1c(O)ccc(C#N)c1-c1cccc(F)c1C#N]

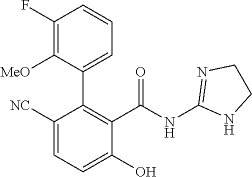 [SMILES: COc1c(F)cccc1-c1c(C#N)ccc(O)c1C(=O)NC1=NCCN1]

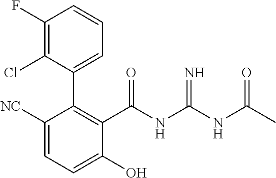 [SMILES: CC(=O)NC(=N)NC(=O)c1c(O)ccc(C#N)c1-c1cccc(F)c1Cl]

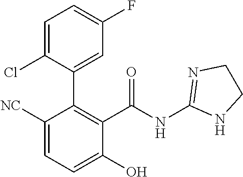 [SMILES: N#Cc1ccc(O)c(C(=O)NC2=NCCN2)c1-c1cc(F)ccc1Cl]

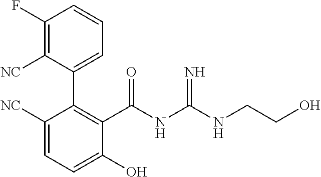 [SMILES: N#Cc1ccc(O)c(C(=O)NC(=N)NCCO)c1-c1cccc(F)c1C#N]

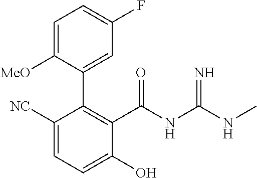 [SMILES: CNC(=N)NC(=O)c1c(O)ccc(C#N)c1-c1cc(F)ccc1OC]

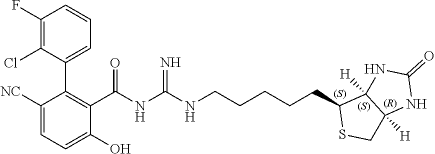

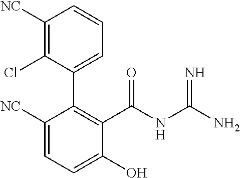 [SMILES: N#Cc1cccc(-c2c(C#N)ccc(O)c2C(=O)NC(=N)N)c1Cl]

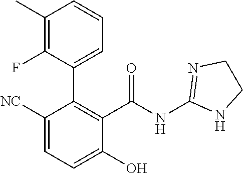 [SMILES: Cc1cccc(-c2c(C#N)ccc(O)c2C(=O)NC2=NCCN2)c1F]

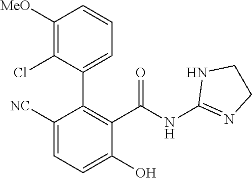 [SMILES: COc1cccc(-c2c(C#N)ccc(O)c2C(=O)NC2=NCCN2)c1Cl]

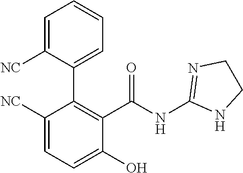 [SMILES: N#Cc1ccccc1-c1c(C#N)ccc(O)c1C(=O)NC1=NCCN1]

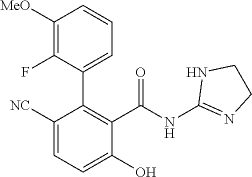 [SMILES: COc1cccc(-c2c(C#N)ccc(O)c2C(=O)NC2=NCCN2)c1F]

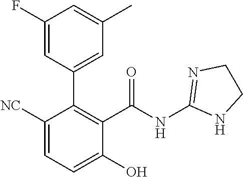 [SMILES: Cc1cc(F)cc(-c2c(C#N)ccc(O)c2C(=O)NC2=NCCN2)c1]

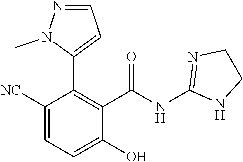 [SMILES: Cn1nccc1-c1c(C#N)ccc(O)c1C(=O)NC1=NCCN1]

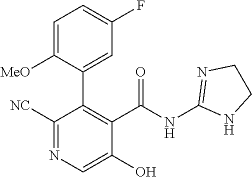 [SMILES: COc1ccc(F)cc1-c1c(C#N)ncc(O)c1C(=O)NC1=NCCN1]

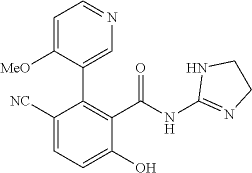 [SMILES: COc1ccncc1-c1c(C#N)ccc(O)c1C(=O)NC1=NCCN1]

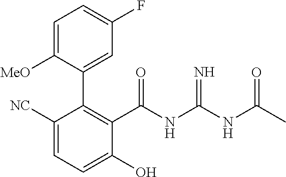 [SMILES: COc1ccc(F)cc1-c1c(C#N)ccc(O)c1C(=O)NC(=N)NC(C)=O]

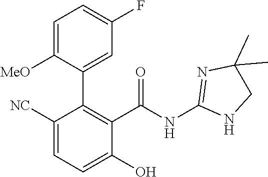 [SMILES: COc1ccc(F)cc1-c1c(C#N)ccc(O)c1C(=O)NC1=NC(C)(C)CN1]

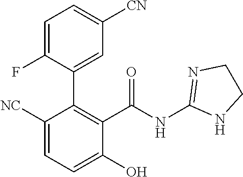 [SMILES: N#Cc1ccc(F)c(-c2c(C#N)ccc(O)c2C(=O)NC2=NCCN2)c1]

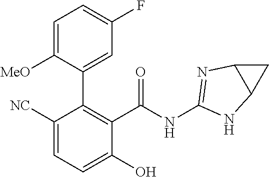 [SMILES: COc1ccc(F)cc1-c1c(C#N)ccc(O)c1C(=O)NC1=NC2CC2N1]

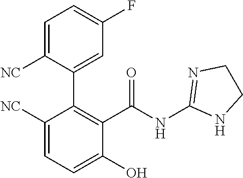 [SMILES: N#Cc1ccc(F)cc1-c1c(C#N)ccc(O)c1C(=O)NC1=NCCN1]

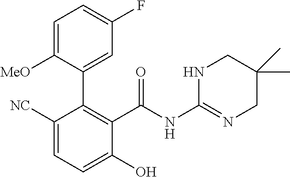 [SMILES: COc1ccc(F)cc1-c1c(C#N)ccc(O)c1C(=O)NC1=NCC(C)(C)CN1]

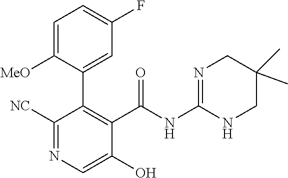 [SMILES: COc1ccc(F)cc1-c1c(C#N)ncc(O)c1C(=O)NC1=NCC(C)(C)CN1]

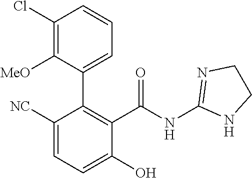 [SMILES: COc1c(Cl)cccc1-c1c(C#N)ccc(O)c1C(=O)NC1=NCCN1]

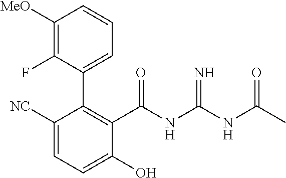 [SMILES: COc1cccc(-c2c(C#N)ccc(O)c2C(=O)NC(=N)NC(C)=O)c1F]

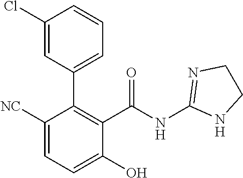 [SMILES: N#Cc1ccc(O)c(C(=O)NC2=NCCN2)c1-c1cccc(Cl)c1]

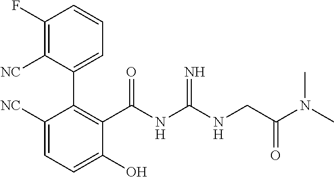 [SMILES: CN(C)C(=O)CNC(=N)NC(=O)c1c(O)ccc(C#N)c1-c1cccc(F)c1C#N]

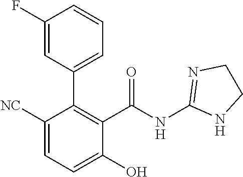 [SMILES: N#Cc1ccc(O)c(C(=O)NC2=NCCN2)c1-c1cccc(F)c1]

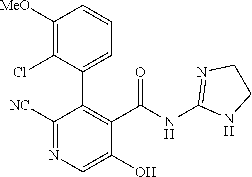 [SMILES: COc1cccc(-c2c(C#N)ncc(O)c2C(=O)NC2=NCCN2)c1Cl]

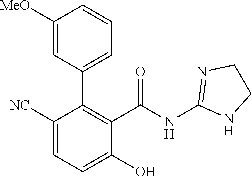 [SMILES: COc1cccc(-c2c(C#N)ccc(O)c2C(=O)NC2=NCCN2)c1]

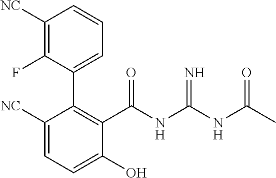 [SMILES: CC(=O)NC(=N)NC(=O)c1c(O)ccc(C#N)c1-c1cccc(C#N)c1F]

52. A pharmaceutical composition, comprising a compound of any one of claims 1-51; and a pharmaceutical acceptable excipient.

53. A method of treating or preventing a disease or disorder associated with a SLC6A8 mutation, comprising administering to a subject in need thereof an effective amount of a compound of any one of claims 1-51.

54. The method of claim 53, wherein the disease or disorder is creatine transporter deficiency.

55. The method of claim 53, wherein the disease or disorder is motor dysfunction.

56. The method of claim 53, wherein the disease or disorder is intellectual disability.

57. The method of claim 53, wherein the disease or disorder is language delay or speech delay.

58. The method of claim 53, wherein the disease or disorder is hypotonia.

59. A method of improving function of a cellular creatine transporter, comprising administering to a subject in need thereof an effective amount of a compound of any one of claims 1-51.

60. The method of claim 59, wherein the creatine transporter is SLC6A8.

61. The method of claim 59 or 60, wherein the creatine transporter is a mutant creatine transporter.

62. A method of decreasing accumulation or the concentration of guanidinoacetic acid or a salt thereof in a cell, comprising administering to a mammal in need thereof a therapeutically effective amount of a compound of any one of claims 1-51.

63. The method of claim 62, wherein the compound decreases intracellular accumulation of guanidinoacetic acid or a salt thereof.

64. The method of claim 62, wherein the compound decreases the intracellular concentration of guanidinoacetic acid or a salt thereof.

65. The method of any one of claims 62-64, wherein the mutant creatine transporter is mutant SLC6A8.

66. The method of any one of claims 62-65, wherein the cell is a brain cell.

67. The method of any one of claims 62-66, wherein the mammal is a male.

68. The method of any one of claims 62-66, wherein the mammal is a female.

69. The method of any one of claims 62-68, wherein the mammal is a primate, equine, bovine, ovine, feline, or canine.

70. The method of any one of claims 62-68, wherein the mammal is a human.

71. A method of increasing transport of guanidinoacetic acid or a salt thereof across the blood-brain barrier, comprising administering to a mammal in need thereof a therapeutically effective amount of a compound of any one of claims 1-51.

72. The method of claim 71, wherein the mutant creatine transporter is mutant SLC6A8.

73. The method of claim 71 or 72, wherein the mammal is a male.

74. The method of claim 71 or 72, wherein the mammal is a female.

75. The method of any one of claims 71-74, wherein the mammal is a primate, equine, bovine, ovine, feline, or canine.

76. The method of any one of claims 71-74, wherein the mammal is a human.

77. A method of treating an inflammatory disease, comprising administering to a mammal in need thereof a therapeutically effective amount of a compound of any one of claims 1-51.

78. The method of claim 77, wherein the inflammatory disease is acute.

79. The method of claim 77, wherein the inflammatory disease is chronic.

80. The method of any one of claims 77-79, wherein the inflammatory disease is selected from inflammatory bowel diseases (for example, ulcerative colitis or Crohn's disease), multiple sclerosis, psoriasis, arthritis, rheumatoid arthritis, osteoarthritis, juvenile arthritis, psoriatic arthritis, reactive arthritis, ankylosing spondylitis, cryopyrin associated periodic syndromes, Muckle-Wells syndrome, familial cold auto-inflammatory syndrome, neonatal-onset multisystem inflammatory disease, TNF receptor associated periodic syndrome, acute and chronic pancreatitis, atherosclerosis, gout, ankylosing spondylitis, fibrotic disorders (for example, hepatic fibrosis or idiopathic pulmonary fibrosis), nephropathy, sarcoidosis, scleroderma, anaphylaxis, diabetes (for example, diabetes mellitus type 1 or diabetes mellitus type 2), diabetic retinopathy, Still's disease, vasculitis, sarcoidosis, pulmonary inflammation, acute respiratory distress syndrome, wet and dry age-related macular degeneration, autoimmune hemolytic syndromes, autoimmune and inflammatory hepatitis, autoimmune neuropathy, autoimmune ovarian failure, autoimmune orchitis, autoimmune thrombocytopenia, silicone implant associated autoimmune disease, Sjogren's syndrome, familial Mediterranean fever, systemic lupus erythematosus, vasculitis syndromes (for example, temporal, Takayasu's and giant cell arteritis, Behçet's disease or Wegener's granulomatosis), vitiligo, secondary hematologic manifestation of autoimmune diseases (for example, anemias), drug-induced autoimmunity, Hashimoto's thyroiditis, hypophysitis, idiopathic thrombocytic pupura, metal-induced autoimmunity, myasthenia gravis, pemphigus, autoimmune deafness (for example, Meniere's disease), Goodpasture's syndrome, Graves' disease, HW-related autoimmune syndromes, Gullain-Barre disease, Addison's disease, anti-phospholipid syndrome, asthma, atopic dermatitis, Celiac disease, Cushing's syndrome, dermatomyositis, idiopathic adrenal adrenal atrophy, idiopathic thrombocytopenia, Kawasaki syndrome, Lambert-Eaton Syndrome, pernicious anemia, pollinosis, polyarteritis nodosa, primary biliary cirrhosis, primary sclerosing cholangitis, Raynaud's, Reiter's Syndrome, relapsing polychondritis, Schmidt's syndrome, thyrotoxidosis, sepsis, septic shock, endotoxic shock, exotoxin-induced toxic shock, gram negative sepsis, toxic shock syndrome, glomerulonephritis, peritonitis, interstitial cystitis, hyperoxia-induced inflammations, chronic obstructive pulmonary disease (COPD), vasculitis, graft vs. host reaction (for example, graft vs. host disease), allograft rejections (for example, acute allograft rejection or chronic allograft rejection), early transplantation rejection (for example, acute allograft rejection), reperfusion injury, pain (for example, acute pain, chronic pain, neuropathic pain, or fibromyalgia), chronic infections, meningitis, encephalitis, myocarditis, gingivitis, post surgical trauma, tissue injury, traumatic brain injury, enterocolitis, sinusitis, uveitis, ocular inflammation, optic neuritis, gastric ulcers, esophagitis, peritonitis, periodontitis, dermatomyositis, gastritis, myositis, polymyalgia, pneumonia and bronchitis.

81. The method of any one of claims 77-80, wherein the mammal is a male.

82. The method of any one of claims 77-80, wherein the mammal is a female.

83. The method of any one of claims 77-80, wherein the mammal is a primate, equine, bovine, ovine, feline, or canine.

84. The method of any one of claims 77-80, wherein the mammal is a human.
